(19) United States
(12) Patent Application Publication
(10) Pub. No.: US 2012/0202794 A1
(43) Pub. Date: Aug. 9, 2012

(54) COMPOUNDS

(75) Inventors: MICHAEL JOSEPH SOFIA, DOYLESTOWN, PA (US); RAMESH KAKARLA, SOUTH GLASTONBURY, CT (US); JIAN LIU, MANALAPAN, NJ (US); DEVAN NADUTHAMBI, PLAINSBORO, NJ (US); RALPH MOSLEY, ROSELLE, NJ (US); HOLLY MICOLOCHICK STEUER, BURLINGTON, NJ (US)

(73) Assignee: PHARMASSET, INC., PRINCETON, NJ (US)

(21) Appl. No.: 13/353,538

(22) Filed: Jan. 19, 2012

Related U.S. Application Data
(60) Provisional application No. 61/435,528, filed on Jan. 24, 2011.

(51) Int. Cl. A61K 31/551; A61K 31/495; A61P 31/12; A61K 31/496; C07D 417/06; C07D 403/06; C07D 241/04; C07D 405/06

(52) U.S. Cl. 514/218; 544/384; 514/255.02; 544/360; 544/379; 514/253.01; 514/254.1; 544/369; 514/254.02; 544/371; 514/254.05; 544/370; 540/492

(57) ABSTRACT

Disclosed herein are compounds useful for treating a viral infection, such as HCV.

### PRIORITY

Priority is claimed to U.S. 61/435,528, filed on Jan. 24, 2011.

### FIELD OF THE INVENTION

Disclosed herein are compounds useful for treating a viral infection, such as HCV.

### BACKGROUND

Hepatitis C virus (HCV) infection is a major health problem that leads to chronic liver disease, such as cirrhosis and hepatocellular carcinoma, in a substantial number of infected individuals, estimated to be 2-15% of the world's population. There are an estimated 4.5 million infected people in the United States alone, according to the U.S. Center for Disease Control. According to the World Health Organization, there are more than 200 million infected individuals worldwide, with at least 3 to 4 million people being infected each year. Once infected, about 20% of people clear the virus, but the rest can harbor HCV the rest of their lives. Ten to twenty percent of chronically infected individuals eventually develop liver-destroying cirrhosis or cancer. The viral disease is transmitted parenterally by contaminated blood and blood products, contaminated needles, or sexually and vertically from infected mothers or carrier mothers to their offspring. Current treatments for HCV infection, which are restricted to immunotherapy with recombinant interferon-α alone or in combination with the nucleoside analog ribavirin, are of limited clinical benefit. Moreover, there is no established vaccine for HCV. Consequently, there is an urgent need for improved therapeutic agents that effectively combat chronic HCV infection.

The HCV virion is an enveloped positive-strand RNA virus with a single oligoribonucleotide genomic sequence of about 9600 bases which encodes a polyprotein of about 3,010 amino acids. The protein products of the HCV gene consist of the structural proteins C, E1, and E2, and the non-structural proteins NS2, NS3, NS4A and NS4B, and NS5A and NS5B. The nonstructural (NS) proteins are believed to provide the catalytic machinery for viral replication. The NS3 protease releases NS5B, the RNA-dependent RNA polymerase from the polyprotein chain. HCV NS5B polymerase is required for the synthesis of a double-stranded RNA from a single-stranded viral RNA that serves as a template in the replication cycle of HCV. Therefore, NS5B polymerase is considered to be an essential component in the HCV replication complex (K. Ishi, et al, Heptology, 1999, 29: 1227-1235; V. Lohmann, et al., Virology, 1998, 249: 108-118). Inhibition of HCV NS5B polymerase prevents formation of the double-stranded HCV RNA and therefore constitutes an attractive approach to the development of HCV-specific antiviral therapies.

HCV belongs to a much larger family of viruses that share many common features.

Flaviviridae Viruses

The Flaviviridae family of viruses comprises at least three distinct genera: pestiviruses, which cause disease in cattle and pigs; flavivruses, which are the primary cause of diseases such as dengue fever and yellow fever; and hepaciviruses, whose sole member is HCV. The flavivirus genus includes more than 68 members separated into groups on the basis of serological relatedness (Calisher et al., J. Gen. Virol, 1993, 70, 37-43). Clinical symptoms vary and include fever, encephalitis and hemorrhagic fever (Fields Virology, Editors: Fields, B. N., Knipe, D. M., and Howley, P. M., Lippincott-Raven Publishers, Philadelphia, Pa., 1996, Chapter 31, 931-959). Flaviviruses of global concern that are associated with human disease include the Dengue Hemorrhagic Fever viruses (DHF), yellow fever virus, shock syndrome and Japanese encephalitis virus (Halstead, S. B., Rev. Infect. Dis., 1984, 6, 251-264; Halstead, S. B., Science, 239:476-481, 1988; Monath, T. P., New Eng. J. Med, 1988, 319, 64 1-643).

The pestivirus genus includes bovine viral diarrhea virus (BVDV), classical swine fever virus (CSFV, also called hog cholera virus) and border disease virus (BDV) of sheep (Moennig, V. et al. Adv. Vir. Res. 1992, 41, 53-98). Pestivirus infections of domesticated livestock (cattle, pigs and sheep) cause significant economic losses worldwide. BVDV causes mucosal disease in cattle and is of significant economic importance to the livestock industry (Meyers, G. and Thiel, H. J., Advances in Virus Research, 1996, 47, 53-118; Moennig V., et al, Adv. Vir. Res. 1992, 41, 53-98). Human pestiviruses have not been as extensively characterized as the animal pestiviruses. However, serological surveys indicate considerable pestivirus exposure in humans.

Pestiviruses and hepaciviruses are closely related virus groups within the Flaviviridae family. Other closely related viruses in this family include the GB virus A, GB virus A-like agents, GB virus-B and GB virus-C (also called hepatitis G virus, HGV). The hepacivirus group (hepatitis C virus; HCV) consists of a number of closely related but genotypically distinguishable viruses that infect humans. There are at least 6 HCV genotypes and more than 50 subtypes. Due to the similarities between pestiviruses and hepaciviruses, combined with the poor ability of hepaciviruses to grow efficiently in cell culture, bovine viral diarrhea virus (BVDV) is often used as a surrogate to study the HCV virus.

The genetic organization of pestiviruses and hepaciviruses is very similar. These positive stranded RNA viruses possess a single large open reading frame (ORF) encoding all the viral proteins necessary for virus replication. These proteins are expressed as a polyprotein that is co- and post-translationally processed by both cellular and virus-encoded proteinases to yield the mature viral proteins. The viral proteins responsible for the replication of the viral genome RNA are located within approximately the carboxy-terminal. Two-thirds of the ORF are termed nonstructural (NS) proteins. The genetic organization and polyprotein processing of the nonstructural protein portion of the ORF for pestiviruses and hepaciviruses is very similar. For both the pestiviruses and hepaciviruses, the mature nonstructural (NS) proteins, in sequential order from the amino-terminus of the nonstructural protein coding region to the carboxy-terminus of the ORF, consist of p7, NS2, NS3, NS4A, NS4B, NS5A, and NS5B.

The NS proteins of pestiviruses and hepaciviruses share sequence domains that are characteristic of specific protein functions. For example, the NS3 proteins of viruses in both groups possess amino acid sequence motifs characteristic of serine proteinases and of helicases (Gorbalenya et al., Nature, 1988, 333, 22; Bazan and Fletterick Virology, 1989, 171, 637-639; Gorbalenya et al., Nucleic Acid Res., 1989, 17, 3889-3897). Similarly, the NS5B proteins of pestiviruses and hepaciviruses have the motifs characteristic of RNA-directed RNA polymerases (Koonin, E. V. and Dolja, V. V., Crir. Rev. Biochem. Molec. Biol. 1993, 28, 375-430).

The actual roles and functions of the NS proteins of pestiviruses and hepaciviruses in the lifecycle of the viruses are directly analogous. In both cases, the NS3 serine proteinase is responsible for all proteolytic processing of polyprotein precursors downstream of its position in the ORF (Wiskerchen and Collett, Virology, 1991, 184, 341-350; Bartenschlager et al., J. Virol. 1993, 67, 3835-3844; Eckart et al. Biochem. Biophys. Res. Comm. 1993, 192, 399-406; Grakoui et al., J. Virol. 1993, 67, 2832-2843; Grakoui et al., Proc. Natl. Acad Sci. USA 1993, 90, 10583-10587; Hijikata et al., J. Virol. 1993, 67, 4665-4675; Tome et al., J. Virol., 1993, 67, 4017-4026). The NS4A protein, in both cases, acts as a cofactor with the NS3 serine protease (Bartenschlager et al., J. Virol. 1994, 68, 5045-5055; Failla et al., J. Virol. 1994, 68, 3753-3760; Xu et al., J. Virol., 1997, 71:53 12-5322). The NS3 protein of both viruses also functions as a helicase (Kim et al., Biochem. Biophys. Res. Comm., 1995, 215, 160-166; Jin and Peterson, Arch. Biochem. Biophys., 1995, 323, 47-53; Warrener and Collett, J. Virol. 1995, 69, 1720-1726). Finally, the NS5B proteins of pestiviruses and hepaciviruses have the predicted RNA-directed RNA polymerases activity (Behrens et al., EMBO, 1996, 15, 12-22; Lechmann et al., J. Virol., 1997, 71, 8416-8428; Yuan et al., Biochem. Biophys. Res. Comm. 1997, 232, 231-235; Hagedorn, PCT WO 97/12033; Zhong et al, J. Virol., 1998, 72, 9365-9369).

A number of potential molecular targets for drug development of direct acting antivirals as anti-HCV therapeutics have now been identified including, but not limited to, the NS2-NS3 autoprotease, the N3 protease, the N3 helicase and the NS5B polymerase. The RNA-dependent RNA polymerase is absolutely essential for replication of the single-stranded, positive sense, RNA genome and this enzyme has elicited significant interest among medicinal chemists. Another auxiliary protein of HCV is referred to as NS4B. However, very few inhibitors of NS4B have been reported. This is in part due to the fact that NS4B is a relatively poorly characterized 27 kDa protein with at least four predicted transmembrane (TM) domains. It is believed that as consequence of polyprotein processing by the NS3-4A protease, the N- and C-terminal parts of NS4B are oriented towards the cytosolic side of the endoplasmic reticulum (ER) membrane. Dvory-Sobol et al. Viruses, 2010, 2, 2481-2492. Furthermore, it is believed that HCV NS4B associates with a number of additional non-structural proteins that permit formation of the so-called “membranous web” structure that facilitates HCV replication. Egger et al. J. Virol. 2002, 76, 5974-5984; Gosert et al. Hepatology 2002, 36, 757-760. In an effort to improve treatment of HCV, it remains of vital interest to identify compounds capable of inhibiting the action of NS4B of HCV.

### SUMMARY

Disclosed herein is a compound or its stereoisomer or its salt thereof represented by the following formula A:

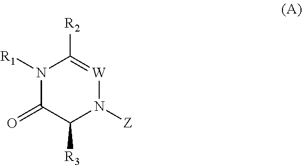

wherein
C [symbol] W is a single-bond or a double-bond, where
   (i) W is CH, when C [symbol] W is a double-bond,
   (ii) W is CH₂, CHR₄, or CHR₅CHR₆ when C [symbol] W is a single-bond,
   or
   (iii) W is absent and the carbon atom of ˜C(—R₂)˜ is bound directly to the nitrogen atom of ˜N(—Z)˜ to form a compound having the structure A′

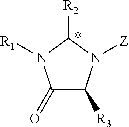

R₁ is selected from among hydrogen, an alkyl, an alkaryl, an acyl, an aryl, and a heteroaryl;
R₂, R₃, R₄, R₅, and R₆ are independently selected from among, hydrogen, an alkyl, an allyl, an alkaryl, halo, a cycloalkyl, an alkylene-cycloalkyl, an alkylenethioalkyl, an alkyleneoxoalkyl, an alkylene-cycloalkyl, an aryl, an alkaryl, a heteroaryl, an alkheteroaryl, COOH, COOalkyl, CONHalkyl, and CON(alkyl)₂; and
Z is ˜Q-Yₘ—B or ˜T-A-Xₙ—B
   where
   Q is —(CH₂)ₒ— (o is 0, 1, 2, or 3) or —C(O)—;
   Y is O, S, or NH and m is 0 or 1;
   T is —(CH₂)ₚ—(C(O))_{q}— (p is 0, 1, 2, or 3 and q is 0 or 1) or CHR₇—C(O)—, where R₇ is a C₁₋₆alkyl;
   X is CH₂, O, NH, or S, with n is 0 or 1;
A is selected from among —CR′═CR″—, —C≡C—, a cycloalkylene, an arylene, and a heteroarylene,
   where R′ and R″ are independently selected from among hydrogen, a C₁₋₆alkyl, and an aryl; and
B is selected from among hydrogen, a C₁₋₆alkyl, a cycloalkyl, an aryl, an aryloxide, a heteroaryl, and a fused ring moiety.

### DETAILED DESCRIPTION OF THE INVENTION

### Definitions

The phrase “a” or “an” entity as used herein refers to one or more of that entity; for example, a compound refers to one or more compounds or at least one compound. As such, the terms “a” (or “an”), “one or more”, and “at least one” can be used interchangeably herein.

The terms “optional” or “optionally” as used herein means that a subsequently described event or circumstance may but need not occur, and that the description includes instances where the event or circumstance occurs and instances in which it does not. For example, “optional bond” means that the bond may or may not be present, and that the description includes single, double, or triple bonds.

The term “stereoisomer” has its plain and ordinary meaning. In some instances, chiral centers are represented by an asterisk “*”.

The term “salts” or “salt thereof” as described herein, refers to a compound comprising a cation and an anion, which can prepared by any process known to one of ordinary skill, e.g., by the protonation of a proton-accepting moiety and/or deprotonation of a proton-donating moiety. Alternatively, the salt can be prepared by a cation/anion metathesis reaction. It should be noted that protonation of the proton-accepting moiety results in the formation of a cationic species in which the charge is balanced by the presence of a anion, whereas deprotonation of the proton-donating moiety results in the formation of an anionic species in which the charge is balanced by the presence of a cation. It is understood that salt formation can occur under synthetic conditions, such as formation of pharmaceutically acceptable salts, or under conditions formed in the body.

The phrase “pharmaceutically acceptable salt” means a salt that is pharmaceutically acceptable. It is understood that the term “pharmaceutically acceptable salt” is encompassed by the expression “salt.” Examples of pharmaceutically acceptable salts include, but are not limited to acid addition salts, formed with inorganic acids such as hydrochloric acid, hydrobromic acid, sulfuric acid, nitric acid, phosphoric acid, and the like; or formed with organic acids such as glycolic acid, pyruvic acid, lactic acid, malonic acid, malic acid, maleic acid, fumaric acid, tartaric acid, citric acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, lauryl sulfuric acid, gluconic acid, glutamic acid, salicylic acid, muconic acid, and the like. Additional examples of anionic radicals of the pharmaceutically acceptable salt include but are not limited to: acetate, benzenesulfonate, benzoate, bicarbonate, bitartrate, bromide, calcium edetate, camsylate (camphorsulfonate), carbonate, chloride, citrate, edetate, edisylate (1,2-ethanedisulfonate), estolate (lauryl sulfate), esylate (ethanesulfonate), fumarate, gluceptate (glucoheptonate), gluconate, glutamate, glycollylarsanilate (p-glycollamidophenylarsonate), hexylresorcinate, hydrabamine (N,N′-di(dehydroabietyl)ethylenediamine), hydroxynaphthoate, iodide, isethionate (2-hydroxyethanesulfonate), lactate, lactobionate, malate, maleate, mandelate, mesylate, methylnitrate, methylsulfate, mucate, napsylate, nitrate, pamoate (embonate), pantothenate, phosphate/diphosphate, polygalacturonate, salicylate, stearate, subacetate, succinate, sulfate, tannate, tartrate, and teoclate (8-chlorotheophyllinate). Basic addition salts formed with the conjugate bases of any of the inorganic acids listed above, wherein the conjugate bases comprise a cationic component selected from among Li⁺, Na⁺, K⁺, Mg²⁺, Ca²⁺, Al³⁺, NH_{g}R″_{4-g}⁺, in which R″ is a C₁₋₃ alkyl and g is a number selected from among 0, 1, 2, 3, or 4. Additional examples of cationic radicals of the pharmaceutically acceptable salt, include but are not limited to: penzathine, phloroprocaine, pholine, piethanolamine, ethylenediamine, meglumine, and procaine.

The term “halo” or “halogen” as used herein, includes chloro, bromo, iodo and fluoro.

The term “alkyl” refers to an unbranched or branched chain, saturated, monovalent hydrocarbon residue containing 1 to 30 carbon atoms. The term “C_{1-M}alkyl” refers to an alkyl comprising 1 to M carbon atoms, where M is an integer having the following values: 2, 3, 4, 5, 6, 7, 8, 9, 10, 11, 12, 13, 14, 15, 16, 17, 18, 19, 20, 21, 22, 23, 24, 25, 26, 27, 28, 29, or 30.

The term “C₁₋₆alkyl” refers to an alkyl containing 1 to 6 carbon atoms. Examples of a C₁₋₆ alkyl group include, but are not limited to, methyl, ethyl, propyl, i-propyl, n-butyl, i-butyl, t-butyl, pentyl, isopentyl, neopentyl, hexyl, etc.

The term “alkylene” refers to an unbranched or branched chain, saturated, monovalent hydrocarbon residue containing 1 to 30 carbon atoms. The term “C_{1-M}alkylene refers to an alkylene radical comprising 1 to M carbon atoms, where M is an integer having the following values: 2, 3, 4, 5, 6, 7, 8, 9, 10, 11, 12, 13, 14, 15, 16, 17, 18, 19, 20, 21, 22, 23, 24, 25, 26, 27, 28, 29, or 30.

The term “C₁₋₆-alkylene” refers to an alkylene radical containing 1 to 6 carbon atoms. Examples of a C₁₋₆-alkylene include, but are not limited to, a methylene (—CH₂—), ethylene (—CH₂CH₂—), methyl-ethylene (—CH(CH₃)CH₂—), propylene (—CH₂CH₂CH₂—), methyl-propylene (—CH(CH₃)CH₂CH₂— or —CH₂CH(CH₃)CH₂—), etc. It is understood that a branched C₁₋₆-alkylene, such as methyl-ethylene or methyl-propylene, contains a chiral center, in which case the individual stereoisomers are contemplated. It is contemplated that a methylene may be substituted with one or two C₁₋₆alkyls.

The term “cycloalkyl” refers to an unsubstituted or substituted carbocycle, in which the carbocycle contains 3 to 10 carbon atoms. In the instance of a substituted carbocycle containing 3 to 10 carbon atoms, the substituents are not to be counted for the carbocycle carbon count. For instance, a cyclohexyl substituted with one or more C₁₋₆-alkyl is still, within the meaning contemplated herein, a C₃₋₆-cycloalkyl. Examples of a C₃₋₆cycloalkyl include, but are not limited to, cyclopropyl (cPr), 2-methyl-cyclopropyl, cyclobutyl (cBu), 2-methyl-cyclobutyl, cyclopentyl (cPn), 2-methyl-cylcopentyl, cyclohexyl (cHx), 2-methyl-cyclohexyl, etc.

The term “C₃₋₆cycloalkyl” refers to an unsubstituted or substituted carbocycle, in which the carbocycle contains 3 to 6 carbon atoms. In the instance of a substituted carbocycle containing 3 to 6 carbon atoms, the substituents are not to be counted for the carbocycle carbon count. For instance, a cyclohexyl substituted with one or more C₁₋₆-alkyl is still, within the meaning contemplated herein, a C₃₋₆-cycloalkyl. Examples of a C₃₋₆cycloalkyl include, but are not limited to, cyclopropyl (cPr), 2-methyl-cyclopropyl, cyclobutyl (cBu), 2-methyl-cyclobutyl, cyclopentyl (cPn), 2-methyl-cylcopentyl, cyclohexyl (cHx), 2-methyl-cyclohexyl, etc.

The term “alkyleneoxoalkyl” refers to an alkylene-O-alkyl, where the terms alkylene and alkyl are as defined herein. A subset of the alkyleneoxoalkyl moiety is a —(C₁₋₆-alkylene)oxo(C₁₋₆alkyl), which includes, but is not limited to, —CH₂OCH₃, etc.

The term “alkylenethioalkyl” refers to an alkylene-S-alkyl, where the terms alkylene and alkyl are as defined herein. A subset of the alkylenethioalkyl moiety is a (C₁₋₆alkylene)thio(C₁₋₆alkyl) moiety, which includes, but is not limited to, —CH₂SCH₃, etc.

The term “alkylene-cycloalkyl” refers to a radical comprised of a cycloalkyl bonded to an alkylene, which are both defined herein, CH₂cPr, CH₂cBu, CH₂cPn, CH₂cHx, CH₂CH₂cPr, CH₂CH₂cBu, CH₂CH₂cPn, CH₂CH₂cHx, etc.

The term “C₁₋₆alkylene-C₃₋₆cycloalkyl” refers to a radical comprised of a C₃₋₆cycloalkyl bonded to a C₁₋₆alkylene, which are both defined herein. An examples of a C₁₋₆alkylene-C₃₋₆cycloalkyl includes, but is not limited to, CH₂cPr, CH₂cBu, CH₂cPn, CH₂cHx, CH₂CH₂cPr, CH₂CH₂cBu, CH₂CH₂cPn, CH₂CH₂cHx, etc.

The term allyl refers to CH₂C═CH₂.

The term alkenyl refers to an unbranched or branched chain, having at least one unsaturated C—C bond, containing 2 to 30 carbon atoms. The term “C_{2-M}alkenyl” refers to an alkenyl chaing having at least one unsaturated C—C bond comprising 2 to M carbon atoms, where M is an integer having the following values: 3, 4, 5, 6, 7, 8, 9, 10, 11, 12, 13, 14, 15, 16, 17, 18, 19, 20, 21, 22, 23, 24, 25, 26, 27, 28, 29, or 30.

The terms “alkaryl” or “alkylaryl” refer to an alkylene group with an aryl substituent, both of which ar defined herein. The term “C₁₋₃alkaryl” refers to a C₁₋₃alkylene group with an aryl substituent. Benzyl is embraced by the term C₁₋₃-alkaryl.

The term “aryl,” as used herein, and unless otherwise specified, refers to substituted or unsubstituted phenyl (Ph), biphenyl, or naphthyl. The aryl group can be substituted with one or more moieties selected from among an alkyl, a cycloalkyl, a halogenated alkyl (e.g., —CHₙF₃₋ₙ, —CH₂CHₙF₃₋ₙ, —CFHCHₙF₃₋ₙ, —CF₂CHₙF₃₋ₙ, etc., with n=0, 1, or 2) hydroxyl, F, Cl, Br, I, phenyl, substituted phenyl (where the substituent is at least one of those described in the present paragraph), —C(O)OH, —C(O)O(alkyl), —C(O)NH₂, —C(O)NH(alkyl), —C(O)N(alkyl)₂, amino, alkylamino, arylamino, alkoxy, halo-alkoxy (e.g., —OCHₙF₃₋ₙ, —OCH₂CHₙF₃₋ₙ, —OCFHCHₙF₃₋ₙ, —OCF₂CHₙF₃₋ₙ, etc., with n=0, 1, or 2), aryloxide (i.e., —Oaryl), nitro, cyano, sulfonic acid, sulfate, —S(alkyl), —S(O)alkyl, —S(O)₂(alkyl), phosphonic acid, phosphate, and phosphonate, either unprotected, or protected as necessary, as known to those skilled in the art, for example, as taught in T. W. Greene and P. G. M. Wuts, “Protective Groups in Organic Synthesis,” 3rd ed., John Wiley & Sons, 1999. Selected examples of aryls include, but are not limited to,

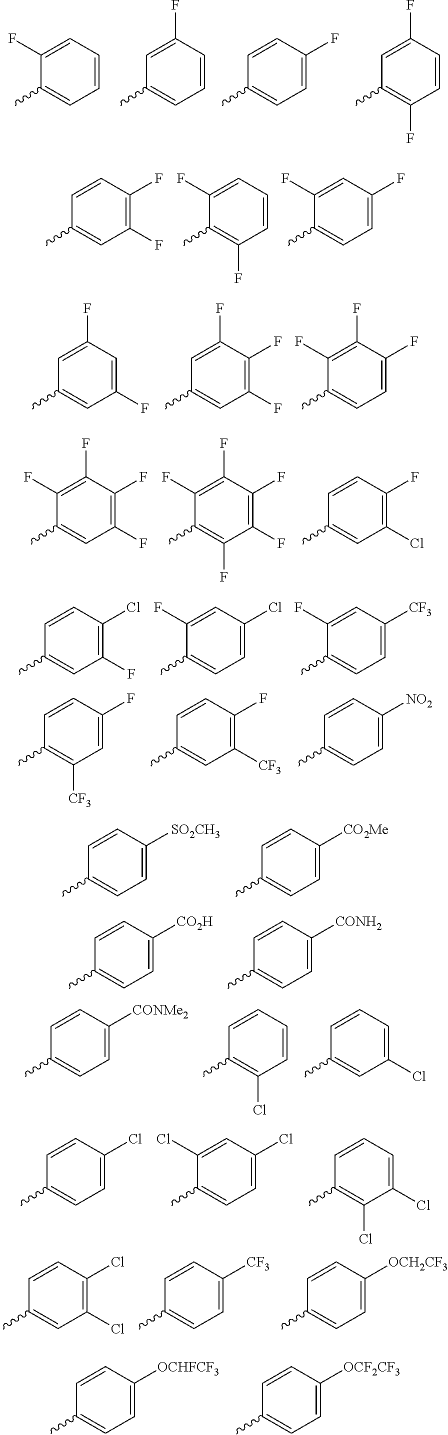

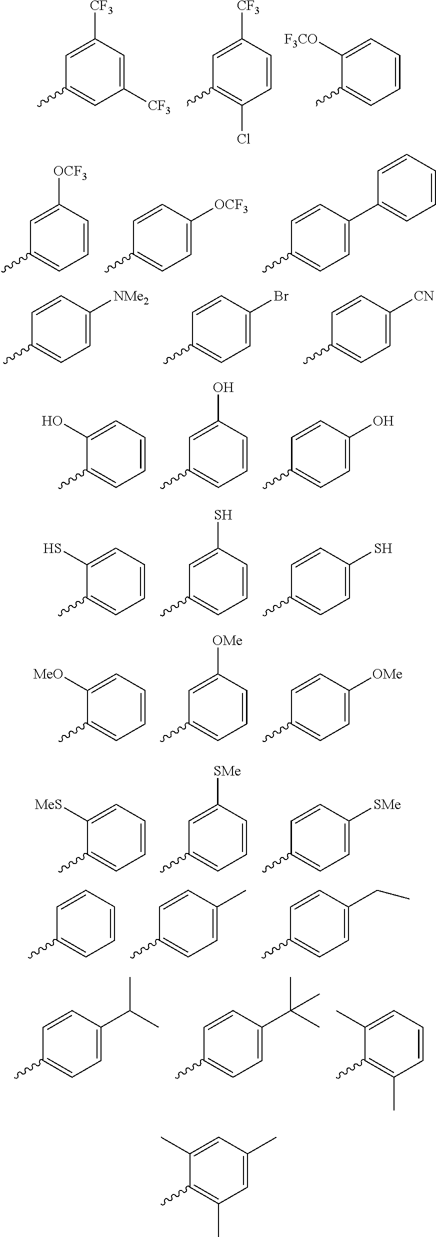

where the point of attachment is represented by a ( [symbol] ).

The term “arylene” is an aryl group bonded to two moieties, such as, e.g., a carbonyl (C(O)) bound to one carbon and an oxazole or a phenyl group bound to another carbon in the arylene ring, in which a specific arylene, phenylene is depicted structurally below

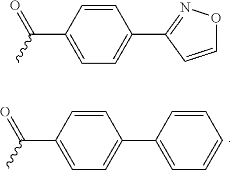

The term “heteroaryl” refers to an unsubstituted or substituted aromatic heterocycle containing carbon, hydrogen, and at least one of N, O, and S. Examples of heteroaryls include, but are not limited to, a pyrrole, an imidazole, a pyrazole, a triazole (1H-1,2,3-triazole, 2H-1,2,3-triazole, 1H-1,2,4-triazole, or 2H-1,2,4-triazole), a tetrazole, a furan, an oxazole, an oxadiazole (1,2,4-oxadiazole or 1,3,4-oxadiazole), a thiophene, a thiazole, an indole, a benzofuran, a benzo[b]thiophene, a 1H-indole, a benzo[d][1,3]-dioxol-n-yl (where n is the point of attachment to the benzo-ring), etc. Additional examples of heteroaryls can be found in T. L. Gilchrist, in “Heterocyclic Chemistry,” John Wiley & Sons, 1985. The heteroaryl group can be substituted with one or more moieties selected from among alkyl, hydroxyl, F, Cl, Br, I, amino, alkylamino, arylamino, alkoxy, aryloxy, nitro, cyano, sulfonic acid, sulfate, phosphonic acid, phosphate, and phosphonate, either unprotected, or protected as necessary, as known to those skilled in the art, for example, as taught in T. W. Greene and P. G. M. Wuts, “Protective Groups in Organic Synthesis,” 3rd ed., John Wiley & Sons, 1999. Examples of heteroaryls include, but are not limited to

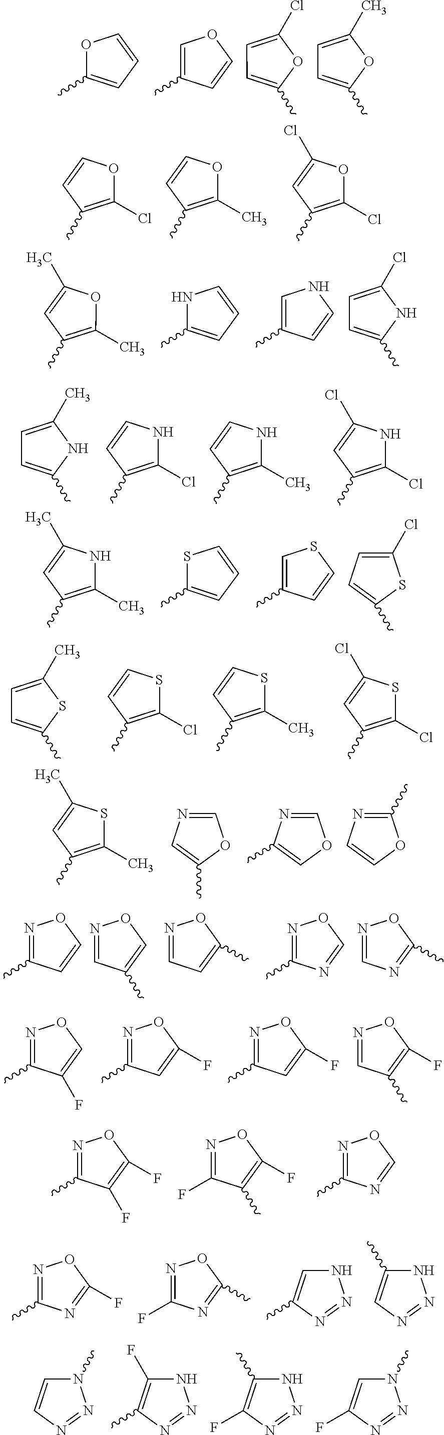

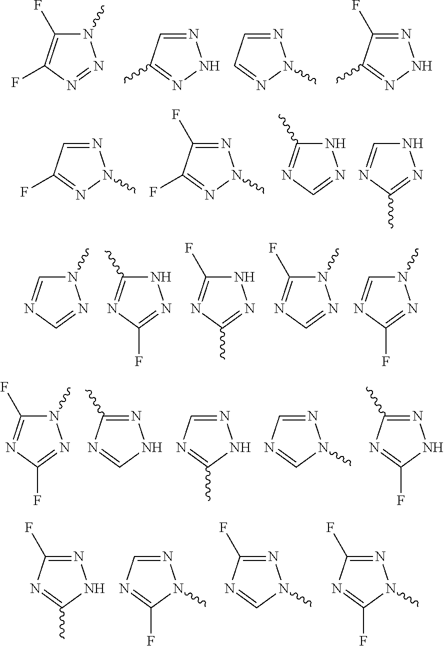

where the point of attachment is represented by a ( [symbol] ).

The term “heteroarylene” is an heteroaryl group bonded to two moieties, such as, e.g., a carbonyl (C(O)) bound to one carbon and an oxazole or a 4-fluorophenyl group bound to another carbon in the heteroarylene ring, in which a specific heteroarylene, oxazolylene is depicted structurally below

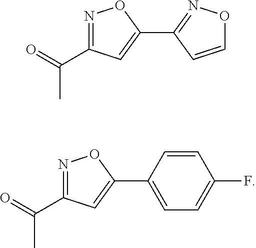

The terms “C₁₋₆alk(heteroaryl)” and “alk(heterocyclyl)” refers to a C₁₋₆-alkylene group with a heteroaryl and heterocyclyl substituent, respectively.

The term “aryloxide,” or “aryloxy” as used herein, and unless otherwise specified, refers to substituted or unsubstituted phenoxide (PhO—), p-phenyl-phenoxide (p-Ph-PhO—), or naphthoxide, preferably the term aryloxide refers to substituted or unsubstituted phenoxide. The aryloxide group can be substituted with one or more moieties selected from among hydroxyl, F, Cl, Br, I, —C(O)(C₁₋₆alkyl), —C(O)O(C₁₋₆alkyl), amino, alkylamino, arylamino, alkoxy, aryloxy, nitro, cyano, sulfonic acid, sulfate, phosphonic acid, phosphate, and phosphonate, either unprotected, or protected as necessary, as known to those skilled in the art, for example, as taught in T. W. Greene and P. G. M. Wuts, “Protective Groups in Organic Synthesis,” 3rd ed., John Wiley & Sons, 1999.

The term “fused ring moiety” refers to two or more rings fused together at adjacent atoms. Examples of fused ring moieties include, but are not limited to:

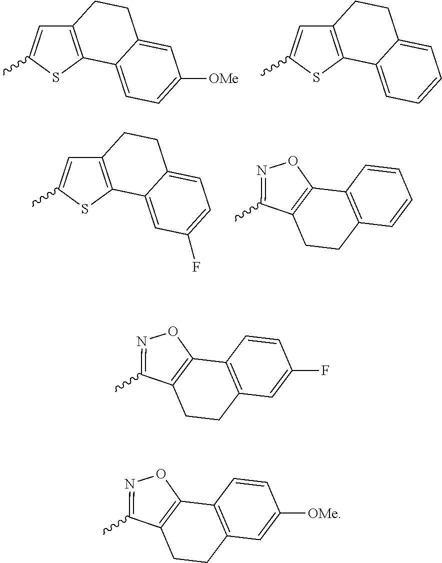

where the point of attachment is represented by a ( [symbol] ).

The term “acyl” refers to a substituent containing a carbonyl moiety and a non-carbonyl moiety and is meant to include an amino-acyl. The carbonyl moiety contains a double-bond between the carbonyl carbon and a heteroatom, where the heteroatom is selected from among O, N and S. When the heteroatom is N, the N is substituted by a C₁₋₆. The non-carbonyl moiety is selected from straight, branched, and cyclic alkyl, which includes, but is not limited to, a straight, branched, or cyclic C₁₋₂₀ alkyl, C₁₋₁₀ alkyl, or a C₁₋₆-alkyl; alkoxyalkyl, including methoxymethyl; aralkyl, including benzyl; aryloxyalkyl, such as phenoxymethyl; or aryl, including phenyl optionally substituted with halogen (F, Cl, Br, I), hydroxyl, C₁ to C₄ alkyl, or C₁ to C₄ alkoxy, sulfonate esters, such as alkyl or aralkyl sulphonyl, including methanesulfonyl, the mono, di or triphosphate ester, trityl or monomethoxytrityl, substituted benzyl, trialkylsilyl (e.g. dimethyl-t-butylsilyl) or diphenylmethylsilyl. When at least one aryl group is present in the non-carbonyl moiety, it is preferred that the aryl group comprises a phenyl group.

The term “C₂₋₇acyl” refers to an acyl group in which the non-carbonyl moiety comprises a C₁₋₆alkyl. Examples of a C₂₋₇-acyl, include, but are not limited to: —C(O)CH₃, —C(O)CH₂CH₃, —C(O)CH(CH₃)₂, —C(O)CH(CH₃)CH₂CH₃, —C(O)C(CH₃)₃, etc.

The expression “R₂-substituent” as used herein refers to the following radicals: CH₃, CH₂CH₃, CH₂CH₂CH₃, CH₂OCH₃, CH₂SCH₃, CH(CH₃)₂, CH(CH₃)(CH₂CH₃), C(CH₃)₃, CH₂CH(CH₃)₂, CH₂C(CH₃)₃, CH═CHCH₃, CH═CH, CH₂cPr, CH₂cBu, CH₂cPn, CH₂cHx, cPr, cBu, cPn, cHx, CH₂Ph,

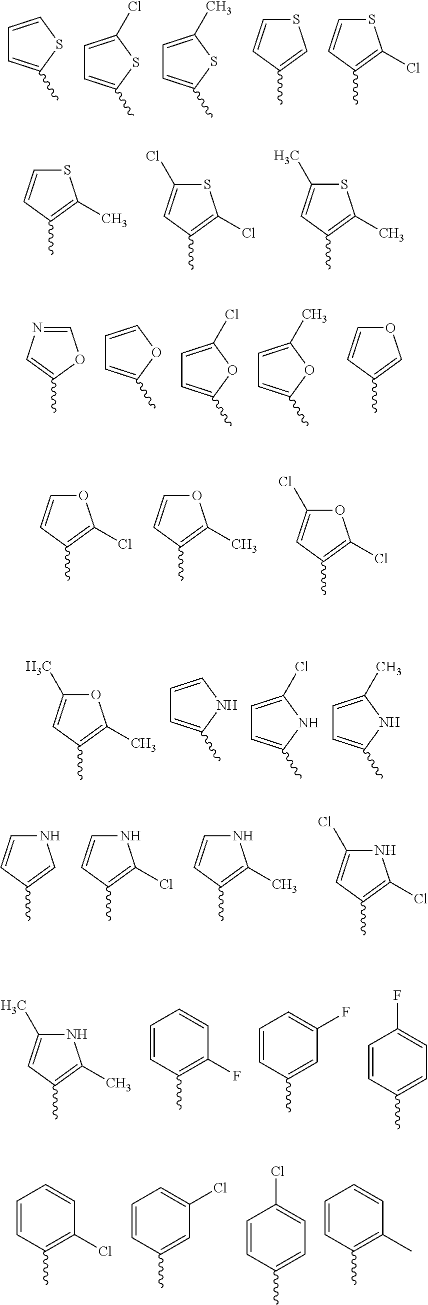

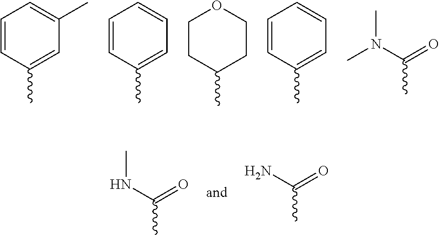

where the point of attachment is represented by a ( [symbol] ).

The expression “A′-ring” as used herein refers to the following radicals:

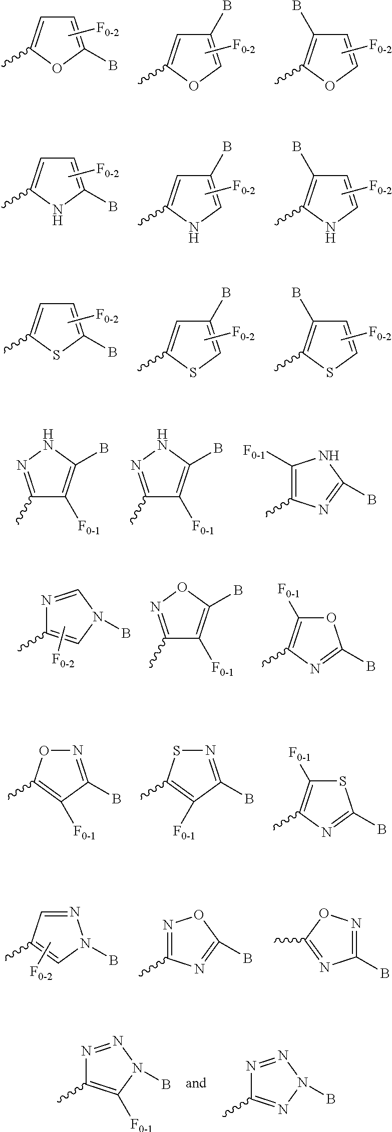

where —B is as defined herein or in the context as used in a particular embodiment or sub-embodiment, —F₀₋₁ bound to a particular carbon-atom denotes a hydrogen atom for —F₀ and a fluorine atom for —F₁, and —F₀₋₂ not bound to particular carbon-atom means that one or more of the carbon-atoms in the A′-ring is bound to any one of a hydrogen atom and a fluorine atom. the point of attachment is represented by a ( [symbol] ).

The expression “B′-ring” as used herein refers to the following radicals:

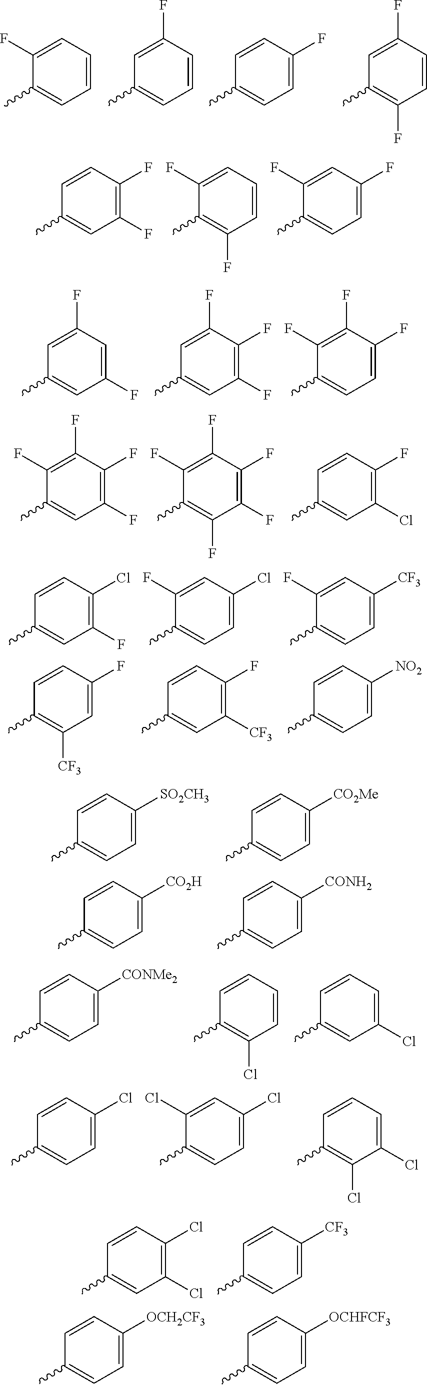

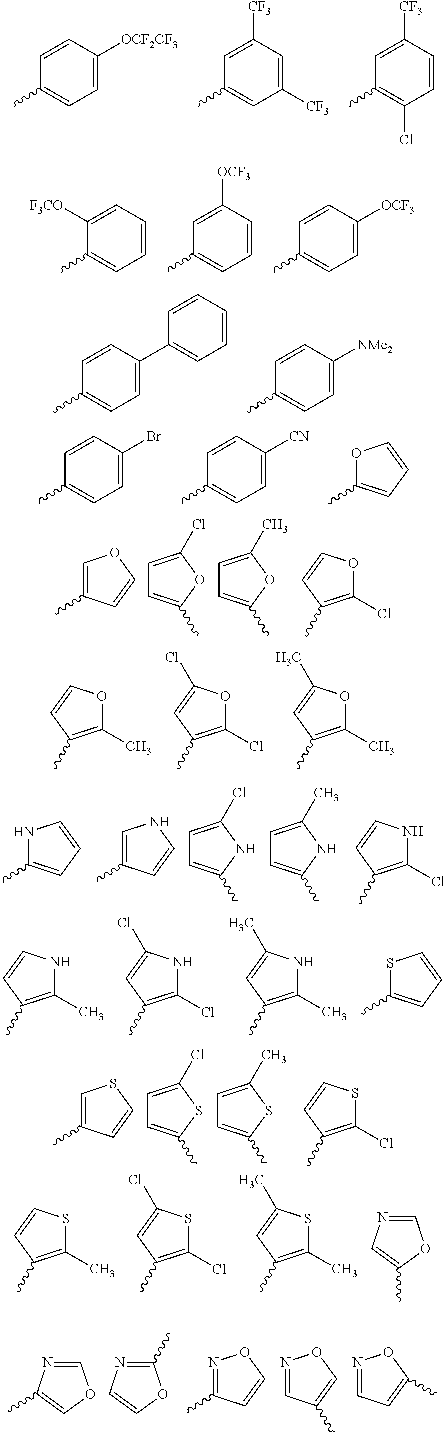

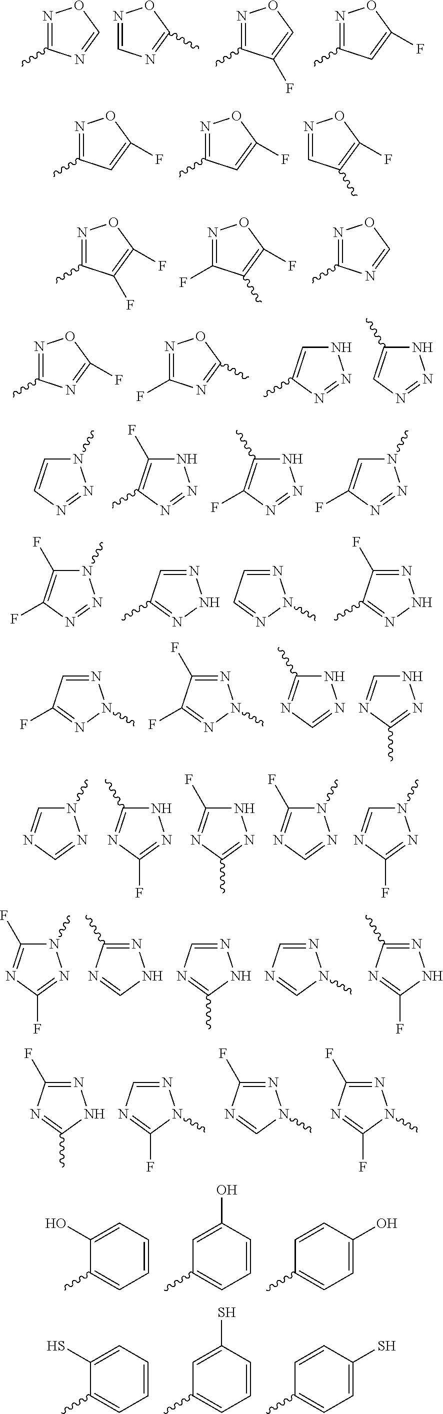

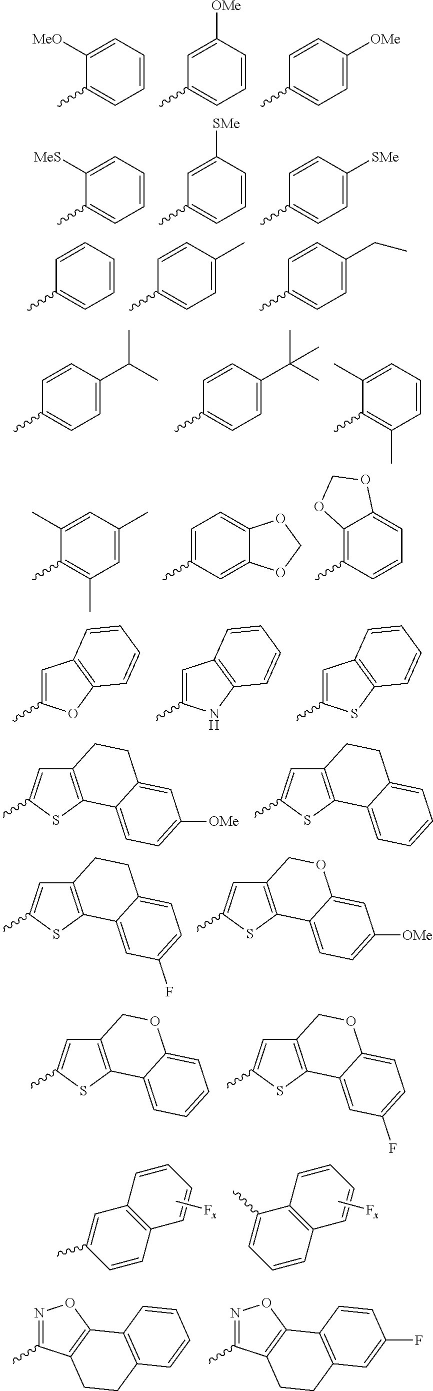

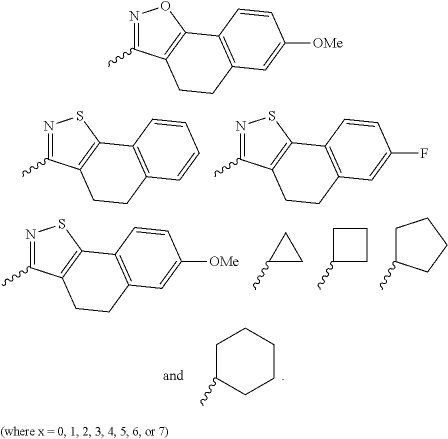

where the point of attachment is represented by a ( [symbol] ).

The term “effective amount” as used herein means an amount required to reduce symptoms of the disease in a subject.

The term “subject,” as used herein means a mammal.

The term “medicament,” as used herein means a substance used in a method of treatment and/or prophylaxis of a subject in need thereof.

The term “preparation” or “dosage form” is intended to include both solid and liquid formulations of the active compound and one skilled in the art will appreciate that an active ingredient can exist in different preparations depending on the desired dose and pharmacokinetic parameters.

The term “excipient” as used herein refers to a compound that is used to prepare a pharmaceutical composition, and is generally safe, non-toxic and neither biologically nor otherwise undesirable, and includes excipients that are acceptable for veterinary use as well as human pharmaceutical use.

As used herein, “treatment” or “treating” is an approach for obtaining beneficial or desired clinical results. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. “Treatment” is an intervention performed with the intention of preventing the development or altering the pathology of a disorder. The term “treatment” of an HCV infection, as used herein, also includes treatment or prophylaxis of a disease or a condition associated with or mediated by HCV infection, or the clinical symptoms thereof.

Disclosed herein is a compound or its stereoisomer or its salt thereof represented by the following formula A:

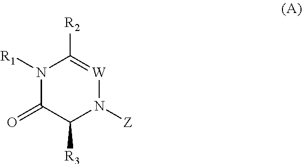

wherein
C [symbol] W is a single-bond or a double-bond, where
   (i) W is CH, when C [symbol] W is a double-bond,
   (ii) W is CH₂, CHR₄, or CHR₅CHR₆ when C [symbol] W is a single-bond, or
   (iii) W is absent and the carbon atom of ˜C(—R₂)˜ is bound directly to the nitrogen atom of ˜N(—Z)˜ to form a compound having the structure A′

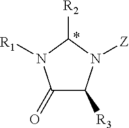

R₁ is selected from among hydrogen, an alkyl, an alkaryl, an acyl, an aryl, and a heteroaryl;
R₂, R₃, R₄, R₅, and R₆ are independently selected from among, hydrogen, an alkyl, an allyl, an alkaryl, halo, a cycloalkyl, an alkylene-cycloalkyl, an alkylenethioalkyl, an alkyleneoxoalkyl, an alkylene-cycloalkyl, an aryl, an alkaryl, a heteroaryl, an alkheteroaryl, COOH, COOalkyl, CONHalkyl, and CON(alkyl)₂; and
Z is ˜Q-Yₘ—B or ˜T-A-Xₙ—B
   where
   Q is —(CH₂)ₒ— (o is 0, 1, 2, or 3) or —C(O)—;
   Y is O, S, or NH and m is 0 or 1;
   T is —(CH₂)ₚ—(C(O))_{q}— (p is 0, 1, 2, or 3 and q is 0 or 1) or CHR₇—C(O)—, where R₇ is a C₁₋₆alkyl;
   X is CH₂, O, NH, or S, with n is 0 or 1;
A is selected from among —CR′═CR″—, —C≡C—, a cycloalkylene, an arylene, and a heteroarylene,
where R′ and R″ are independently selected from among hydrogen, a C₁₋₆alkyl, and an aryl; and
B is selected from among hydrogen, a C₁₋₆alkyl, a cycloalkyl, an aryl, an aryloxide, a heteroaryl, and a fused ring moiety.

### EMBODIMENTS

In a first embodiment of compound (A) or its stereoisomer or its salt thereof, W is CH and C [symbol] W is a double-bond to provide a compound represented by the formula I:

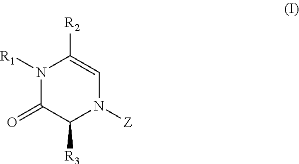

wherein
R₁ is selected from among hydrogen, an alkyl, an alkaryl, an acyl, an aryl, and a heteroaryl;
R₂ and R₃ are independently selected from among, hydrogen, an alkyl, allyl, halo, a cycloalkyl, an alkylene-cycloalkyl, an alkylenethioalkyl, an alkyleneoxoalkyl, an alkylene-cycloalkyl, an aryl, an alkaryl, a heteroaryl, an alkheteroaryl, COOH, COOalkyl, CONHalkyl, CON(alkyl)₂; and
Z is selected from among ˜Q-Yₘ—B and ˜T-A-Xₙ—B
   where
   Q is —(CH₂)ₒ— (o is 0, 1, 2, or 3) or

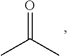

Y is O, S, or NH and m is 0 or 1,
T is —(CH₂)ₚ—(C(O))_{q}— (with p is 0, 1, 2, or 3 and q is 0 or 1) or CHR₇, where R₇ is a C₁₋₆alkyl,
X is CH₂, O, NH, or S, with n is 0 or 1
A is selected from among —CR′═CR″—, —C≡C—, a cycloalkylene, an arylene, and a heteroarylene,
   where R′ and R″ are independently selected from among hydrogen, a C₁₋₆alkyl, and an aryl; and
B is selected from among hydrogen, a C₁₋₆alkyl, a cycloalkyl, an aryl, a heteroaryl, and a fused ring moiety.

In a first aspect of the first embodiment Z is ˜Q-Yₘ—B and Q is —(CH₂)ₒ— (o is 0, 1, 2, or 3), and the compound of formula I is represented by formula (I-1)

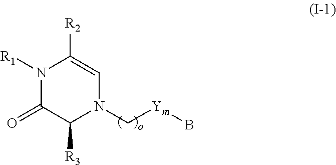

wherein
R₁ is selected from among hydrogen, an alkyl, an alkaryl, an acyl, an aryl, and a heteroaryl;
R₂ and R₃ are independently selected from among, hydrogen, an alkyl, allyl, halo, a cycloalkyl, an alkylene-cycloalkyl, an alkylenethioalkyl, an alkyleneoxoalkyl, an alkylene-cycloalkyl, an aryl, an alkaryl, a heteroaryl, an alkheteroaryl, COOH, COOalkyl, CONHalkyl, CON(alkyl)₂;
Y is O, S, or NH and m is 0 or 1; and
B is selected from among hydrogen, a C₁₋₆alkyl, a cycloalkyl, an aryl, a heteroaryl, and a fused ring moiety.

In a second aspect of the first embodiment Z is ˜Q-Yₘ—B and Q is —(CH₂)ₒ— (o is 0, 1, 2, or 3), and the compound of formula I is represented by formula (I-1)

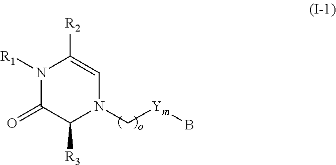

wherein
R₁ is selected from among hydrogen and a C₁₋₆alkyl;
R₂ and R₃ are independently selected from among, hydrogen, an alkyl, allyl, halo, a cycloalkyl, an alkylene-cycloalkyl, an alkylenethioalkyl, an alkyleneoxoalkyl, an alkylene-cycloalkyl, an aryl, an alkaryl, a heteroaryl, and an alkheteroaryl;
Y is O, S, or NH and m is 0 or 1; and
B is selected from among hydrogen, a C₁₋₆alkyl, a cycloalkyl, an aryl, a heteroaryl, and a fused ring moiety.

In a third aspect of the first embodiment Z is ˜Q-Yₘ—B and Q is —(CH₂)ₒ— (o is 0, 1, 2, or 3), and the compound of formula I is represented by formula (I-1)

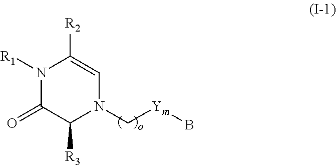

wherein
R₁ is selected from among hydrogen and a C₁₋₆alkyl;
R₂ and R₃ are independently selected from among, hydrogen, a C₁₋₆alkyl, allyl, a C₃₋₆cycloalkyl, a C₁₋₆alkyleneC₃₋₆cycloalkyl, a —(C₁₋₆-alkylene)oxo(C₁₋₆alkyl), a (C₁₋₆alkylene)thio(C₁₋₆alkyl), an aryl, a C₁₋₃alkaryl, a heteroaryl, and a C₁₋₆alkheteroaryl;
Y is O, S, or NH and m is 0 or 1; and
B is selected from among hydrogen, a C₁₋₆alkyl, a C₃₋₆cycloalkyl, an aryl, a heteroaryl, and a fused ring moiety.

In a fourth aspect of the first embodiment Z is ˜Q-Yₘ—B and Q is —(CH₂)ₒ— (o is 0, 1, 2, or 3), and the compound of formula I is represented by formula (I-1)

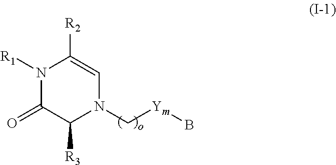

wherein
R₁ is hydrogen;
R₂ and R₃ are independently selected from among, hydrogen, a C₁₋₆alkyl, allyl, a C₃₋₆cycloalkyl, a C₁₋₆alkyleneC₃₋₆cycloalkyl, a —(C₁₋₆-alkylene)oxo(C₁₋₆alkyl), a (C₁₋₆alkylene)thio(C₁₋₆alkyl), an aryl, a C₁₋₃alkaryl, a heteroaryl, and a C₁₋₆alkheteroaryl;
Y is O, S, or NH and m is 0 or 1; and
B is selected from among hydrogen, a C₁₋₆alkyl, a C₃₋₆cycloalkyl, an aryl, a heteroaryl, and a fused ring moiety.

In a fifth aspect of the first embodiment Z is ˜Q-Yₘ—B and Q is —(CH₂)ₒ— (o is 0, 1, 2, or 3), and m is 0, and the compound of formula I is represented by formula (I-2)

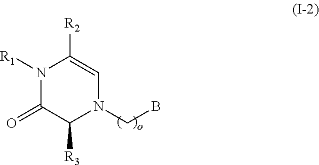

wherein
R₁ is selected from among hydrogen, an alkyl, an alkaryl, an acyl, an aryl, and a heteroaryl;
R₂ and R₃ are independently selected from among, hydrogen, an alkyl, allyl, halo, a cycloalkyl, an alkylene-cycloalkyl, an alkylenethioalkyl, an alkyleneoxoalkyl, an alkylene-cycloalkyl, an aryl, an alkaryl, a heteroaryl, an alkheteroaryl, COOH, COOalkyl, CONHalkyl, CON(alkyl)₂;
Y is O, S, or NH and m is 0 or 1; and
B is selected from among hydrogen, a C₁₋₆alkyl, a cycloalkyl, an aryl, a heteroaryl, and a fused ring moiety.

In a sixth aspect of the first embodiment Z is ˜Q-Yₘ—B and Q is —(CH₂)ₒ— (o is 0, 1, 2, or 3), and m is 0, and the compound of formula I is represented by formula (I-2)

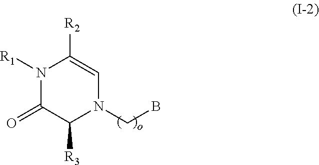

wherein
R₁ is selected from among hydrogen and a C₁₋₆alkyl;
R₂ and R₃ are independently selected from among, hydrogen, an alkyl, allyl, halo, a cycloalkyl, an alkylene-cycloalkyl, an alkylenethioalkyl, an alkyleneoxoalkyl, an alkylene-cycloalkyl, an aryl, an alkaryl, a heteroaryl, and an alkheteroaryl;
Y is O, S, or NH and m is 0 or 1; and
B is selected from among hydrogen, a C₁₋₆alkyl, a cycloalkyl, an aryl, a heteroaryl, and a fused ring moiety.

In a seventh aspect of the first embodiment Z is ˜Q-Yₘ—B and Q is —(CH₂)ₒ— (o is 0, 1, 2, or 3), and m is 0, and the compound of formula I is represented by formula (I-2)

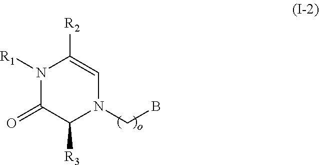

wherein
R₁ is selected from among hydrogen and a C₁₋₆alkyl;
R₂ and R₃ are independently selected from among, hydrogen, a C₁₋₆alkyl, allyl, a C₃₋₆cycloalkyl, a C₁₋₆alkyleneC₃₋₆cycloalkyl, a —(C₁₋₆-alkylene)oxo(C₁₋₆alkyl), a (C₁₋₆alkylene)thio(C₁₋₆alkyl), an aryl, a C₁₋₃alkaryl, a heteroaryl, and a C₁₋₆alkheteroaryl;
Y is O, S, or NH and m is 0 or 1; and
B is selected from among hydrogen, a C₁₋₆alkyl, a C₃₋₆cycloalkyl, an aryl, a heteroaryl, and a fused ring moiety.

In an eighth aspect of the first embodiment Z is ˜Q-Yₘ—B and Q is —(CH₂)ₒ— (o is 0, 1, 2, or 3), and m is 0, and the compound of formula I is represented by formula (I-2)

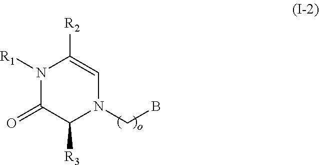

wherein
R₁ is hydrogen;
R₂ and R₃ are independently selected from among, hydrogen, a C₁₋₆alkyl, allyl, a C₃₋₆cycloalkyl, a C₁₋₆alkyleneC₃₋₆cycloalkyl, a —(C₁₋₆-alkylene)oxo(C₁₋₆alkyl), a (C₁₋₆alkylene)thio(C₁₋₆alkyl), an aryl, a C₁₋₃alkaryl, a heteroaryl, and a C₁₋₆alkheteroaryl;
Y is O, S, or NH and m is 0 or 1; and
B is selected from among hydrogen, a C₁₋₆alkyl, a C₃₋₆cycloalkyl, an aryl, a heteroaryl, and a fused ring moiety.

In a ninth aspect of the first embodiment Z is ˜Q-Yₘ—B and Q is —(C(O)), and the compound of formula I is represented by formula (I-3)

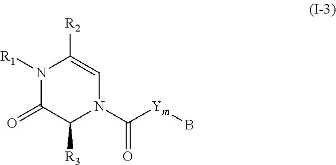

wherein
R₁ is selected from among hydrogen, an alkyl, an alkaryl, an acyl, an aryl, and a heteroaryl;
R₂ and R₃ are independently selected from among, hydrogen, an alkyl, allyl, halo, a cycloalkyl, an alkylene-cycloalkyl, an alkylenethioalkyl, an alkyleneoxoalkyl, an alkylene-cycloalkyl, an aryl, an alkaryl, a heteroaryl, an alkheteroaryl, COOH, COOalkyl, CONHalkyl, CON(alkyl)₂;
Y is CH₂, O, S, or NH and m is 0 or 1; and
B is selected from among hydrogen, a C₁₋₆alkyl, a cycloalkyl, an aryl, a heteroaryl, and a fused ring moiety.

In a 10th aspect of the first embodiment Z is ˜Q-Yₘ—B and Q is —(C(O)), and the compound of formula I is represented by formula (I-3)

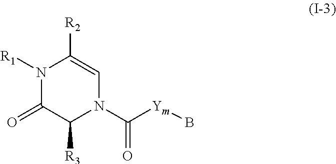

wherein
R₁ is selected from among hydrogen and a C₁₋₆alkyl;
R₂ and R₃ are independently selected from among, hydrogen, an alkyl, allyl, halo, a cycloalkyl, an alkylene-cycloalkyl, an alkylenethioalkyl, an alkyleneoxoalkyl, an alkylene-cycloalkyl, an aryl, an alkaryl, a heteroaryl, and an alkheteroaryl;
Y is CH₂, O, S, or NH and m is 0 or 1; and
B is selected from among hydrogen, a C₁₋₆alkyl, a cycloalkyl, an aryl, a heteroaryl, and a fused ring moiety.

In an 11th aspect of the first embodiment Z is ˜Q-Yₘ—B and Q is —(C(O)), and the compound of formula I is represented by formula (I-3)

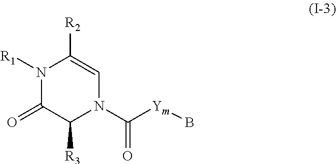

wherein
R₁ is selected from among hydrogen and a C₁₋₆alkyl;
R₂ and R₃ are independently selected from among, hydrogen, a C₁₋₆alkyl, allyl, a C₃₋₆cycloalkyl, a C₁₋₆alkyleneC₃₋₆cycloalkyl, a —(C₁₋₆-alkylene)oxo(C₁₋₆alkyl), a (C₁₋₆alkylene)thio(C₁₋₆alkyl), an aryl, a C₁₋₃alkaryl, a heteroaryl, and a C₁₋₆alkheteroaryl;
Y is CH₂, O, S, or NH and m is 0 or 1; and
B is selected from among hydrogen, a C₁₋₆alkyl, a C₃₋₆cycloalkyl, an aryl, a heteroaryl, and a fused ring moiety.

In a 12th aspect of the first embodiment Z is ˜Q-Yₘ—B and Q is —(C(O)), and the compound of formula I is represented by formula (I-3)

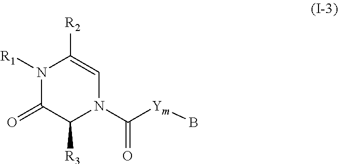

wherein
R₁ is hydrogen;
R₂ and R₃ are independently selected from among, hydrogen, a C₁₋₆alkyl, allyl, a C₃₋₆cycloalkyl, a C₁₋₆alkyleneC₃₋₆cycloalkyl, a —(C₁₋₆-alkylene)oxo(C₁₋₆alkyl), a (C₁₋₆alkylene)thio(C₁₋₆alkyl), an aryl, a C₁₋₃alkaryl, a heteroaryl, and a C₁₋₆alkheteroaryl;
Y is CH₂, O, S, or NH and m is 0 or 1; and
B is selected from among hydrogen, a C₁₋₆alkyl, a C₃₋₆cycloalkyl, an aryl, a heteroaryl, and a fused ring moiety.

In a 13th aspect of the first embodiment Z is ˜Q-Yₘ—B and Q is —(C(O)) and m is 0, and the compound of formula I is represented by formula (I-4)

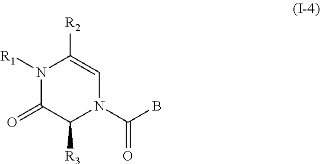

wherein
R₁ is selected from among hydrogen, an alkyl, an alkaryl, an acyl, an aryl, and a heteroaryl;
R₂ and R₃ are independently selected from among, hydrogen, an alkyl, allyl, halo, a cycloalkyl, an alkylene-cycloalkyl, an alkylenethioalkyl, an alkyleneoxoalkyl, an alkylene-cycloalkyl, an aryl, an alkaryl, a heteroaryl, an alkheteroaryl, COOH, COOalkyl, CONHalkyl, CON(alkyl)₂; and
B is selected from among hydrogen, a C₁₋₆alkyl, a cycloalkyl, an aryl, a heteroaryl, and a fused ring moiety.

In a 14th aspect of the first embodiment Z is ˜Q-Yₘ—B and Q is —(C(O)) and m is 0, and the compound of formula I is represented by formula (I-4)

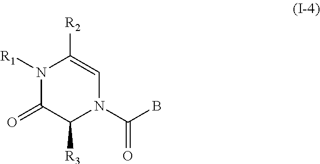

wherein
R₁ is selected from among hydrogen and a C₁₋₆alkyl;
R₂ and R₃ are independently selected from among, hydrogen, an alkyl, allyl, halo, a cycloalkyl, an alkylene-cycloalkyl, an alkylenethioalkyl, an alkyleneoxoalkyl, an alkylene-cycloalkyl, an aryl, an alkaryl, a heteroaryl, and an alkheteroaryl; and
B is selected from among hydrogen, a C₁₋₆alkyl, a cycloalkyl, an aryl, a heteroaryl, and a fused ring moiety.

In a 15th aspect of the first embodiment Z is ˜Q-Yₘ—B and Q is —(C(O)) and m is 0, and the compound of formula I is represented by formula (I-4)

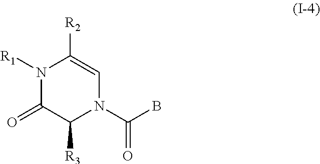

wherein
R₁ is selected from among hydrogen and a C₁₋₆alkyl;
R₂ and R₃ are independently selected from among, hydrogen, a C₁₋₆alkyl, allyl, a C₃₋₆cycloalkyl, a C₁₋₆alkyleneC₃₋₆cycloalkyl, a —(C₁₋₆-alkylene)oxo(C₁₋₆alkyl), a (C₁₋₆alkylene)thio(C₁₋₆alkyl), an aryl, a C₁₋₃alkaryl, a heteroaryl, and a C₁₋₆alkheteroaryl; and
B is selected from among hydrogen, a C₁₋₆alkyl, a C₃₋₆cycloalkyl, an aryl, a heteroaryl, and a fused ring moiety.

In a 16th aspect of the first embodiment Z is ˜Q-Yₘ—B and Q is —(C(O)) and m is 0, and the compound of formula I is represented by formula (I-4)

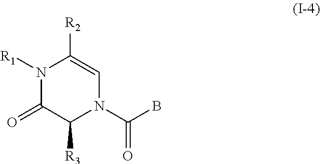

wherein
R₁ is hydrogen;
R₂ and R₃ are independently selected from among, hydrogen, a C₁₋₆alkyl, allyl, a C₃₋₆cycloalkyl, a C₁₋₆alkyleneC₃₋₆cycloalkyl, a —(C₁₋₆-alkylene)oxo(C₁₋₆alkyl), a (C₁₋₆alkylene)thio(C₁₋₆alkyl), an aryl, a C₁₋₃alkaryl, a heteroaryl, and a C₁₋₆alkheteroaryl; and
B is selected from among hydrogen, a C₁₋₆alkyl, a C₃₋₆cycloalkyl, an aryl, a heteroaryl, and a fused ring moiety.

In a second embodiment of compound (A) or its stereoisomer or its salt thereof, W is CH₂, CHR₄, or CHR₅CHR₆ and C [symbol] W is a single-bond to provide a compound represented by formula (II):

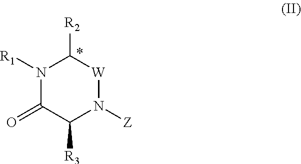

wherein
R₁ is selected from among hydrogen, an alkyl, an alkaryl, an acyl, an aryl, and a heteroaryl;
R₂, R₃, R₄, R₅, and R₆ are independently selected from among, hydrogen, an alkyl, an allyl, an alkaryl, halo, a cycloalkyl, an alkylene-cycloalkyl, an alkylenethioalkyl, an alkyleneoxoalkyl, an alkylene-cycloalkyl, an aryl, an alkaryl, a heteroaryl, an alkheteroaryl, COOH, COOalkyl, CONHalkyl, and CON(alkyl)₂; and
Z is selected from among ˜Q-Yₘ—B and ˜T-A-Xₙ—B
   where
   Q is —(CH₂)ₒ— (o is 0, 1, 2, or 3) or —C(O)—;
   Y is O, S, or NH and m is 0 or 1;
   T is —(CH₂)ₚ—(C(O))_{q}—, (p is 0, 1, 2, or 3 and q is 0 or 1) or CHR₇, where R₇ is a C₁₋₆alkyl;
   X is CH₂, O, NH, or S, with n is 0 or 1;
   A is selected from among —CR′═CR″—, —C≡C—, a cycloalkylene, an arylene, and a heteroarylene,
      where R′ and R″ are independently selected from among hydrogen, a C₁₋₆alkyl, and an aryl; and
   B is selected from among hydrogen, a C₁₋₆alkyl, a cycloalkyl, an aryl, an aryloxide, a heteroaryl, and a fused ring moiety.

In a first aspect of the second embodiment W is CH₂, CHR₄, or CHR₅CHR₆, Z is ˜Q-Yₘ—B and Q is —(CH₂)ₒ— (o is 0, 1, 2, or 3), and the compound of formula II is represented by formula (II-1)

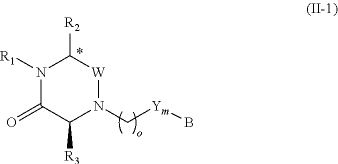

wherein
R₁ is selected from among hydrogen, an alkyl, an alkaryl, an acyl, an aryl, and a heteroaryl;
R₂, R₃, R₄, R₅, and R₆ are independently selected from among, hydrogen, an alkyl, allyl, halo, a cycloalkyl, an alkylene-cycloalkyl, an alkylenethioalkyl, an alkyleneoxoalkyl, an alkylene-cycloalkyl, an aryl, an alkaryl, a heteroaryl, an alkheteroaryl, COOH, COOalkyl, CONHalkyl, and CON(alkyl)₂;
Y is O, S, or NH and m is 0 or 1; and
B is selected from among hydrogen, a C₁₋₆alkyl, a cycloalkyl, an aryl, a heteroaryl, and a fused ring moiety.

In a second aspect of the second embodiment W is CH₂, CHR₄, or CHR₅CHR₆, Z is ˜Q-Yₘ—B and Q is —(CH₂)ₒ— (o is 0, 1, 2, or 3), and the compound of formula II is represented by formula (II-1)

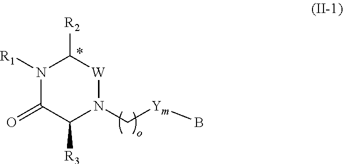

wherein
R₁ is selected from among hydrogen and a C₁₋₆alkyl;
R₂, R₃, R₄, R₅, and R₆ are independently selected from among, hydrogen, an alkyl, an allyl, an alkaryl, halo, a cycloalkyl, an alkylene-cycloalkyl, an alkylenethioalkyl, an alkyleneoxoalkyl, an alkylene-cycloalkyl, an aryl, an alkaryl, a heteroaryl, an alkheteroaryl, COOH, COOalkyl, CONHalkyl, and CON(alkyl)₂;
Y is O, S, or NH and m is 0 or 1; and
B is selected from among hydrogen, a C₁₋₆alkyl, a cycloalkyl, an aryl, a heteroaryl, and a fused ring moiety.

In a third aspect of the second embodiment W is CH₂, CHR₄, or CHR₅CHR₆, Z is ˜Q-Yₘ—B and Q is —(CH₂)ₒ— (o is 0, 1, 2, or 3), and the compound of formula II is represented by formula (II-1)

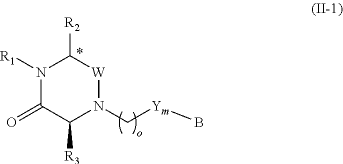

wherein
R₁ is selected from among hydrogen and a C₁₋₆alkyl;
R₂, R₃, R₄, R₅, and R₆ are independently selected from among, hydrogen, an alkyl, an allyl, an alkaryl, halo, a cycloalkyl, an alkylene-cycloalkyl, an alkylenethioalkyl, an alkyleneoxoalkyl, an alkylene-cycloalkyl, an aryl, an alkaryl, a heteroaryl, an alkheteroaryl, COOH, COOalkyl, CONHalkyl, and CON(alkyl)₂;
Y is O, S, or NH and m is 0 or 1; and
B is selected from among hydrogen, a C₁₋₆alkyl, a C₃₋₆cycloalkyl, an aryl, a heteroaryl, and a fused ring moiety.

In a fourth aspect of the second embodiment W is CH₂, CHR₄, or CHR₅CHR₆, Z is ˜Q-Yₘ—B and Q is —(CH₂)ₒ— (o is 0, 1, 2, or 3), and the compound of formula II is represented by formula (II-1)

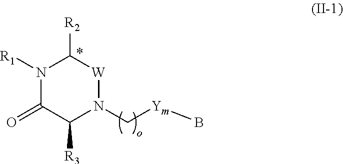

wherein
R₁ is hydrogen;
R₂, R₃, R₄, R₅, and R₆ are independently selected from among, hydrogen, an alkyl, an allyl, an alkaryl, halo, a cycloalkyl, an alkylene-cycloalkyl, an alkylenethioalkyl, an alkyleneoxoalkyl, an alkylene-cycloalkyl, an aryl, an alkaryl, a heteroaryl, an alkheteroaryl, COOH, COOalkyl, CONHalkyl, and CON(alkyl)₂;
Y is O, S, or NH and m is 0 or 1; and
B is selected from among hydrogen, a C₁₋₆alkyl, a C₃₋₆cycloalkyl, an aryl, a heteroaryl, and a fused ring moiety.

In a fifth aspect of the second embodiment W is CH₂, CHR₄, or CHR₅CHR₆, Z is ˜Q-Yₘ—B and Q is —(CH₂)ₒ— (o is 0, 1, 2, or 3), and the compound of formula II is represented by formula (II-1)

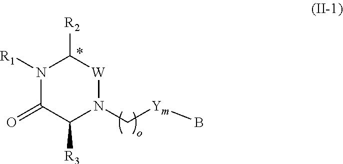

wherein
R₁ is hydrogen;
R₂ is selected from among an R₂-substituent, as defined herein;
R₃ is selected from among CH₃, CH₂CH₃, CH₂CH₂CH₃, CH₂OCH₃, CH₂SCH₃, CH(CH₃)₂, CH(CH₃)(CH₂CH₃), C(CH₃)₃, CH₂CH(CH₃)₂, CH₂C(CH₃)₃, CH═CHCH₃, CH═CH, CH₂cPr, CH₂cBu, CH₂cPn, CH₂cHx, cPr, cBu, cPn, cHx, and CH₂Ph;
each of R₄, R₅, and R₆ is hydrogen;
Y is O, S, or NH and m is 0 or 1; and
B is selected from a B′-ring, as defined herein.

In a sub-aspect of each of the first, second, third, fourth, and fifth aspects of the second embodiment, W is CH₂ and R₂ has a configuration as shown in formula (II-1′)

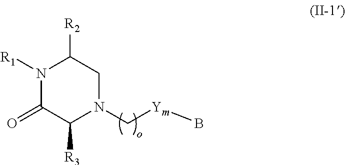

and R₃ is selected from among its respective listing in each of the above-mentioned aspects.

In a sixth aspect of the second embodiment W is CH₂, CHR₄, or CHR₅CHR₆, Z is ˜Q-Yₘ—B, m is 0, and Q is —(CH₂)ₒ— (o is 0, 1, 2, or 3), and the compound of formula II is represented by formula (II-2)

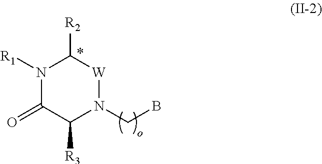

wherein
R₁ is selected from among hydrogen, an alkyl, an alkaryl, an acyl, an aryl, and a heteroaryl;
R₂, R₃, R₄, R₅, and R₆ are independently selected from among hydrogen, an alkyl, allyl, halo, a cycloalkyl, an alkylene-cycloalkyl, an alkylenethioalkyl, an alkyleneoxoalkyl, an alkylene-cycloalkyl, an aryl, an alkaryl, a heteroaryl, an alkheteroaryl, COOH, COOalkyl, CONHalkyl, CON(alkyl)₂; and
B is selected from among hydrogen, a C₁₋₆alkyl, a cycloalkyl, an aryl, a heteroaryl, and a fused ring moiety.

In a seventh aspect of the second embodiment W is CH₂, CHR₄, or CHR₅CHR₆, Z is ˜Q-Yₘ—B, m is 0, and Q is —(CH₂)ₒ— (o is 0, 1, 2, or 3), and the compound of formula II is represented by formula (II-2)

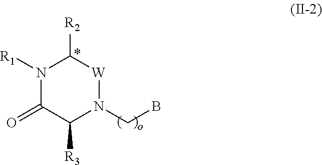

wherein
R₁ is selected from among hydrogen and a C₁₋₆alkyl;
R₂, R₃, R₄, R₅, and R₆ are independently selected from among hydrogen, an alkyl, allyl, halo, a cycloalkyl, an alkylene-cycloalkyl, an alkylenethioalkyl, an alkyleneoxoalkyl, an alkylene-cycloalkyl, an aryl, an alkaryl, a heteroaryl, and an alkheteroaryl; and
B is selected from among hydrogen, a C₁₋₆alkyl, a cycloalkyl, an aryl, a heteroaryl, and a fused ring moiety.

In an eighth aspect of the second embodiment W is CH₂, CHR₄, or CHR₅CHR₆, Z is ˜Q-Yₘ—B, m is 0, and Q is —(CH₂)ₒ— (o is 0, 1, 2, or 3), and the compound of formula II is represented by formula (II-2)

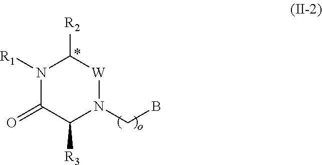

wherein
R₁ is selected from among hydrogen and a C₁₋₆alkyl;
R₂, R₃, R₄, R₅, and R₆ are independently selected from among hydrogen, a C₁₋₆alkyl, allyl, a C₃₋₆cycloalkyl, a C₁₋₆alkyleneC₃₋₆cycloalkyl, a (C₁₋₆-alkylene)oxo(C₁₋₆alkyl), a (C₁₋₆alkylene)thio(C₁₋₆alkyl), an aryl, a C₁₋₃alkaryl, a heteroaryl, and a C₁₋₆alkheteroaryl; and
B is selected from among hydrogen, a C₁₋₆alkyl, a C₃₋₆cycloalkyl, an aryl, a heteroaryl, and a fused ring moiety.

In a ninth aspect of the second embodiment W is CH₂, CHR₄, or CHR₅CHR₆, Z is ˜Q-Yₘ—B, m is 0, and Q is —(CH₂)ₒ— (o is 0, 1, 2, or 3), and the compound of formula II is represented by formula (II-2)

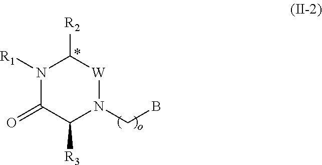

wherein
R₁ is hydrogen;
R₂, R₃, R₄, R₅, and R₆ are independently selected from among hydrogen, a C₁₋₆alkyl, allyl, a C₃₋₆cycloalkyl, a C₁₋₆alkyleneC₃₋₆cycloalkyl, a (C₁₋₆-alkylene)oxo(C₁₋₆alkyl), a (C₁₋₆alkylene)thio(C₁₋₆alkyl), an aryl, a C₁₋₃alkaryl, a heteroaryl, and a C₁₋₆alkheteroaryl; and
B is selected from among hydrogen, a C₁₋₆alkyl, a C₃₋₆cycloalkyl, an aryl, a heteroaryl, and a fused ring moiety.

In a 10th aspect of the second embodiment W is CH₂, CHR₄, or CHR₅CHR₆, Z is ˜Q-Yₘ—B, m is 0, and Q is —(CH₂)ₒ— (o is 0, 1, 2, or 3), and the compound of formula II is represented by formula (II-2)

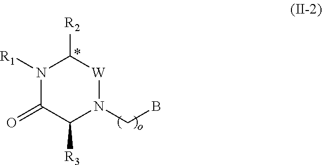

wherein
R₁ is hydrogen;
R₂ is selected from among an R₂-substituent, as defined herein;
R₃ selected from among CH₃, CH₂CH₃, CH₂CH₂CH₃, CH₂OCH₃, CH₂SCH₃, CH(CH₃)₂, CH(CH₃)(CH₂CH₃), C(CH₃)₃, CH₂CH(CH₃)₂, CH₂C(CH₃)₃, CH═CHCH₃, CH═CH, CH₂cPr, CH₂cBu, CH₂cPn, CH₂cHx, cPr, cBu, cPn, cHx, and CH₂Ph;
each of R₄, R₅, and R₆ is hydrogen;
B is selected from a B′-ring, as defined herein.

In a sub-aspect of each of the sixth, seventh, eighth, ninth, and 10th aspects of the second embodiment, W is CH₂ and R₂ has a configuration as shown in formula (II-2′)

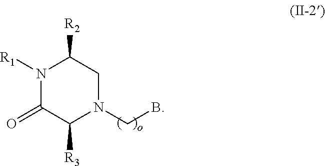

and R₃ is selected from among its respective listing in each of the above-mentioned aspects.

In a 11th aspect of the second embodiment W is CH₂, CHR₄, or CHR₅CHR₆, Z is ˜Q-Yₘ—B and Q is —(C(O))—, and the compound of formula II is represented by formula (II-3)

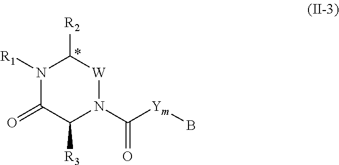

wherein
R₁ is selected from among hydrogen, an alkyl, an alkaryl, an acyl, an aryl, and a heteroaryl;
R₂, R₃, R₄, R₅, and R₆ are independently selected from among hydrogen, an alkyl, allyl, halo, a cycloalkyl, an alkylene-cycloalkyl, an alkylenethioalkyl, an alkyleneoxoalkyl, an alkylene-cycloalkyl, an aryl, an alkaryl, a heteroaryl, an alkheteroaryl, COOH, COOalkyl, CONHalkyl, and CON(alkyl)₂;
Y is O, S, or NH and m is 0 or 1; and
B is selected from among hydrogen, a C₁₋₆alkyl, a cycloalkyl, an aryl, a heteroaryl, and a fused ring moiety.

In a 12th aspect of the second embodiment W is CH₂, CHR₄, or CHR₅CHR₆, Z is ˜Q-Yₘ—B and Q is —(C(O))—, and the compound of formula II is represented by formula (II-3)

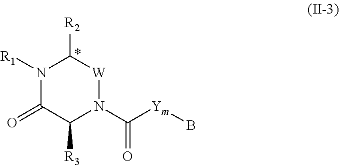

wherein
R₁ is selected from among hydrogen and a C₁₋₆alkyl;
R₂, R₃, R₄, R₅, and R₆ are independently selected from among hydrogen, an alkyl, allyl, halo, a cycloalkyl, an alkylene-cycloalkyl, an alkylenethioalkyl, an alkyleneoxoalkyl, an alkylene-cycloalkyl, an aryl, an alkaryl, a heteroaryl, and an alkheteroaryl;
Y is O, S, or NH and m is 0 or 1; and
B is selected from among hydrogen, a C₁₋₆alkyl, a cycloalkyl, an aryl, a heteroaryl, and a fused ring moiety.

In an 13th aspect of the second embodiment W is CH₂, CHR₄, or CHR₅CHR₆, Z is ˜Q-Yₘ—B and Q is —(C(O))—, and the compound of formula II is represented by formula (II-3)

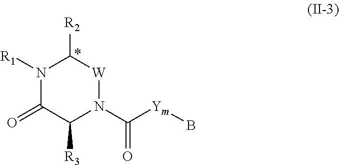

wherein
R₁ is selected from among hydrogen and a C₁₋₆alkyl;
R₂, R₃, R₄, R₅, and R₆ are independently selected from among hydrogen, a C₁₋₆alkyl, allyl, a C₃₋₆cycloalkyl, a C₁₋₆alkyleneC₃₋₆cycloalkyl, a (C₁₋₆-alkylene)oxo(C₁₋₆alkyl), a (C₁₋₆alkylene)thio(C₁₋₆alkyl), an aryl, a C₁₋₃alkaryl, a heteroaryl, and a C₁₋₆alkheteroaryl;
Y is O, S, or NH and m is 0 or 1; and
B is selected from among hydrogen, a C₁₋₆alkyl, a C₃₋₆cycloalkyl, an aryl, a heteroaryl, and a fused ring moiety.

In a 14th aspect of the second embodiment W is CH₂, CHR₄, or CHR₅CHR₆, Z is ˜Q-Yₘ—B and Q is —(C(O))—, and the compound of formula II is represented by formula (II-3)

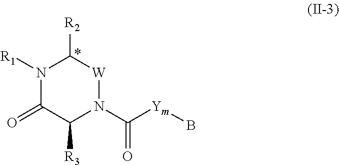

wherein
R₁ is hydrogen;
R₂, R₃, R₄, R₅, and R₆ are independently selected from among hydrogen, a C₁₋₆alkyl, allyl, a C₃₋₆cycloalkyl, a C₁₋₆alkyleneC₃₋₆cycloalkyl, a (C₁₋₆-alkylene)oxo(C₁₋₆alkyl), a (C₁₋₆alkylene)thio(C₁₋₆alkyl), an aryl, a C₁₋₃alkaryl, a heteroaryl, and a C₁₋₆alkheteroaryl;
Y is O, S, or NH and m is 0 or 1; and
B is selected from among hydrogen, a C₁₋₆alkyl, a C₃₋₆cycloalkyl, an aryl, a heteroaryl, and a fused ring moiety.

In a 15th aspect of the second embodiment W is CH₂, CHR₄, or CHR₅CHR₆, Z is ˜Q-Yₘ—B and Q is —(C(O))—, and the compound of formula II is represented by formula (II-3)

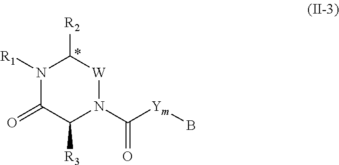

wherein
R₁ is hydrogen;
R₂ is selected from among an R₂-substituent, as defined herein;
R₃ selected from among CH₃, CH₂CH₃, CH₂CH₂CH₃, CH₂OCH₃, CH₂SCH₃, CH(CH₃)₂, CH(CH₃)(CH₂CH₃), C(CH₃)₃, CH₂CH(CH₃)₂, CH₂C(CH₃)₃, CH═CHCH₃, CH═CH, CH₂cPr, CH₂cBu, CH₂cPn, CH₂cHx, cPr, cBu, cPn, cHx, and CH₂Ph;
each of R₄, R₅, and R₆ is hydrogen;
Y is O, S, or NH and m is 0 or 1; and
B is selected from a B′-ring, as defined herein.

In a sub-aspect of each of the 11th, 12th, 13th, 14th, and 15th aspects of the second embodiment, W is CH₂ and R₂ has a configuration as shown in formula (II-3′)

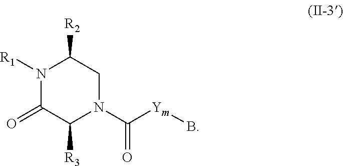

and R₃ is selected from among its respective listing in each of the above-mentioned aspects.

In a 16th aspect of the second embodiment W is CH₂, CHR₄, or CHR₅CHR₆, Z is ˜Q-Yₘ—B, and Q is —C(O)—, and m is 0, and the compound of formula II is represented by formula (II-4)

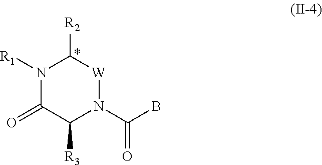

wherein
R₁ is selected from among hydrogen, an alkyl, an alkaryl, an acyl, an aryl, and a heteroaryl;
R₂, R₃, R₄, R₅, and R₆ are independently selected from among hydrogen, an alkyl, allyl, halo, a cycloalkyl, an alkylene-cycloalkyl, an alkylenethioalkyl, an alkyleneoxoalkyl, an alkylene-cycloalkyl, an aryl, an alkaryl, a heteroaryl, an alkheteroaryl, COOH, COOalkyl, CONHalkyl, and CON(alkyl)₂; and
B is selected from among hydrogen, a C₁₋₆alkyl, a cycloalkyl, an aryl, a heteroaryl, and a fused ring moiety.

In a 17th aspect of the second embodiment W is CH₂, CHR₄, or CHR₅CHR₆, Z is ˜Q-Yₘ—B, and Q is —C(O)—, and m is 0, and the compound of formula II is represented by formula (II-4)

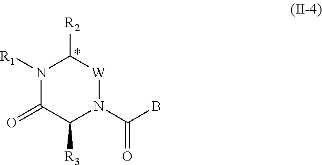

wherein
R₁ is selected from among hydrogen and a C₁₋₆alkyl;
R₂, R₃, R₄, R₅, and R₆ are independently selected from among hydrogen, an alkyl, allyl, halo, a cycloalkyl, an alkylene-cycloalkyl, an alkylenethioalkyl, an alkyleneoxoalkyl, an alkylene-cycloalkyl, an aryl, an alkaryl, a heteroaryl, and an alkheteroaryl; and
B is selected from among hydrogen, a C₁₋₆alkyl, a cycloalkyl, an aryl, a heteroaryl, and a fused ring moiety.

In a 18th aspect of the second embodiment W is CH₂, CHR₄, or CHR₅CHR₆, Z is ˜Q-Yₘ—B, and Q is —C(O)—, and m is 0, and the compound of formula II is represented by formula (II-4)

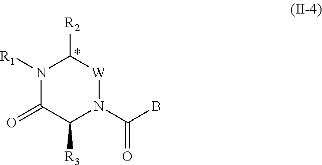

wherein
R₁ is selected from among hydrogen and a C₁₋₆alkyl;
R₂, R₃, R₄, R₅, and R₆ are independently selected from among hydrogen, a C₁₋₆alkyl, allyl, a C₃₋₆cycloalkyl, a C₁₋₆alkyleneC₃₋₆cycloalkyl, a (C₁₋₆-alkylene)oxo(C₁₋₆alkyl), a (C₁₋₆alkylene)thio(C₁₋₆alkyl), an aryl, a C₁₋₃alkaryl, a heteroaryl, and a C₁₋₆alkheteroaryl; and
B is selected from among hydrogen, a C₁₋₆alkyl, a C₃₋₆cycloalkyl, an aryl, a heteroaryl, and a fused ring moiety.

In an 19th aspect of the second embodiment W is CH₂, CHR₄, or CHR₅CHR₆, Z is ˜Q-Yₘ—B, and Q is —C(O)—, and m is 0, and the compound of formula II is represented by formula (II-4)

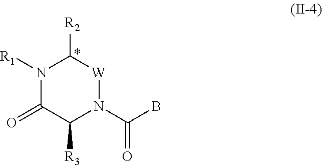

wherein
R₁ is hydrogen;
R₂, R₃, R₄, R₅, and R₆ are independently selected from among hydrogen, a C₁₋₆alkyl, allyl, a C₃₋₆cycloalkyl, a C₁₋₆alkyleneC₃₋₆cycloalkyl, a (C₁₋₆-alkylene)oxo(C₁₋₆alkyl), a (C₁₋₆alkylene)thio(C₁₋₆alkyl), an aryl, a C₁₋₃alkaryl, a heteroaryl, and a C₁₋₆alkheteroaryl; and
B is selected from among hydrogen, a C₁₋₆alkyl, a C₃₋₆cycloalkyl, an aryl, a heteroaryl, and a fused ring moiety.

In a 20th aspect of the second embodiment W is CH₂, CHR₄, or CHR₅CHR₆, Z is ˜Q-Yₘ—B, and Q is —C(O)—, and m is 0, and the compound of formula II is represented by formula (II-4)

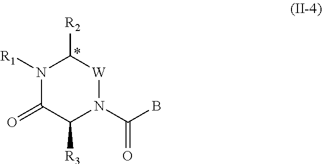

wherein
R₁ is hydrogen;
R₂ is selected from among an R₂-substituent, as defined herein; and
R₃ selected from among CH₃, CH₂CH₃, CH₂CH₂CH₃, CH₂OCH₃, CH₂SCH₃, CH(CH₃)₂, CH(CH₃)(CH₂CH₃), C(CH₃)₃, CH₂CH(CH₃)₂, CH₂C(CH₃)₃, CH═CHCH₃, CH═CH, CH₂cPr, CH₂cBu, CH₂cPn, CH₂cHx, cPr, cBu, cPn, cHx, and CH₂Ph;
each of R₄, R₅, and R₆ is hydrogen; and
B is selected from a B′-ring, as defined herein.

In a sub-aspect of each of the 16th, 17th, 18th, 19th, and 20th aspects of the second embodiment, W is CH₂ and R₂ has a configuration as shown in formula (II-4′)

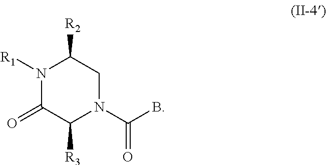

and R₃ is selected from among its respective listing in each of the above-mentioned aspects.

In a 21st aspect of the second embodiment W is CH₂, CHR₄, or CHR₅CHR₆, Z is ˜T-A-Xₙ—B, and T is —(CH₂)ₚ—(C(O))_{q}— (p is 0, 1, 2, or 3 and q is 0 or 1), and the compound of formula II is represented by formula (II-5)

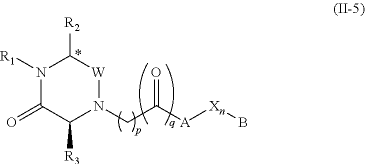

wherein
R₁ is selected from among hydrogen, an alkyl, an alkaryl, an acyl, an aryl, and a heteroaryl;
R₂, R₃, R₄, R₅, and R₆ are independently selected from among hydrogen, an alkyl, allyl, halo, a cycloalkyl, an alkylene-cycloalkyl, an alkylenethioalkyl, an alkyleneoxoalkyl, an alkylene-cycloalkyl, an aryl, an alkaryl, a heteroaryl, an alkheteroaryl, COOH, COOalkyl, CONHalkyl, and CON(alkyl)₂;
X is CH₂, O, S, or NH and n is 0 or 1; and
A is selected from among —CR′═CR″—, —C≡C—, a cycloalkylene, an arylene, and a heteroarylene,
   where R′ and R″ are independently selected from among hydrogen, a C₁₋₆alkyl, and an aryl; and
B is selected from among hydrogen, a C₁₋₆alkyl, a cycloalkyl, an aryl, a heteroaryl, and a fused ring moiety.

In an 22nd aspect of the second embodiment W is CH₂, CHR₄, or CHR₅CHR₆, Z is ˜T-A-Xₙ—B, and T is —(CH₂)ₚ—(C(O))_{q}— (p is 0, 1, 2, or 3 and q is 0 or 1), and the compound of formula II is represented by formula (II-5)

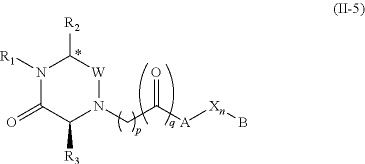

wherein
R₁ is selected from among hydrogen, an alkyl, an alkaryl, an acyl, an aryl, and a heteroaryl;
R₂, R₃, R₄, R₅, and R₆ are independently selected from among hydrogen, an alkyl, allyl, halo, a cycloalkyl, an alkylene-cycloalkyl, an alkylenethioalkyl, an alkyleneoxoalkyl, an alkylene-cycloalkyl, an aryl, an alkaryl, a heteroaryl, and an alkheteroaryl;
X is CH₂, O, S, or NH and n is 0 or 1; and
A is selected from among —CR′═CR″—, —C≡C—, a cycloalkylene, an arylene, and a heteroarylene,
   where R′ and R″ are independently selected from among hydrogen, a C₁₋₆alkyl, and an aryl; and
B is selected from among hydrogen, a C₁₋₆alkyl, a cycloalkyl, an aryl, a heteroaryl, and a fused ring moiety.

In a 23rd aspect of the second embodiment W is CH₂, CHR₄, or CHR₅CHR₆, Z is ˜T-A-Xₙ—B, T is —(CH₂)ₚ—(C(O))_{q}— (p is 0, 1, 2, or 3 and q is 0 or 1), and the compound of formula II is represented by formula (II-5)

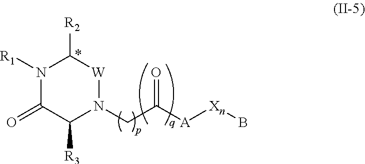

wherein
R₁ is selected from among hydrogen, a C₁₋₆alkyl and a C₁₋₃alkaryl;
R₂, R₃, R₄, R₅, and R₆ are independently selected from among hydrogen, a C₁₋₆alkyl, allyl, a C₃₋₆cycloalkyl, a C₁₋₆alkylene-C₃₋₆cycloalkyl, a C₁₋₆alkylenethioC₁₋₆alkyl, a C₁₋₆alkyleneoxoC₁₋₆alkyl, a C₁₋₆alkylene-C₃₋₆cycloalkyl, an aryl, a C₁₋₃alkaryl, a heteroaryl, and a C₁₋₃alkheteroaryl;
X is CH₂, O, S, or NH and n is 0 or 1; and
A is selected from among —CR′═CR″—, —C≡C—, a C₃₋₆cycloalkylene, an arylene, and a heteroarylene,
   where R′ and R″ are independently selected from among hydrogen, a C₁₋₆alkyl, and an aryl; and
B is selected from among hydrogen, a C₁₋₆alkyl, a C₃₋₆cycloalkyl, an aryl, a heteroaryl, and a fused ring moiety.

In a 24th aspect of the second embodiment W is CH₂, CHR₄, or CHR₅CHR₆, Z is ˜T-A-Xₙ—B, and T is —(CH₂)ₚ—(C(O))_{q}— (p is 0, 1, 2, or 3 and q is 0 or 1), and the compound of formula II is represented by formula (II-5)

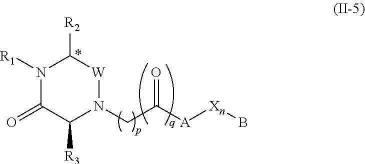

wherein
R₁ is hydrogen or a C₁₋₆alkyl;
R₂, R₃, R₄, R₅, and R₆ are independently selected from among hydrogen, a C₁₋₆alkyl, a C₃₋₆cycloalkyl, a C₁₋₆alkylene-C₃₋₆cycloalkyl, a C₁₋₆alkylenethioC₁₋₆alkyl, a C₁₋₆alkyleneoxoC₁₋₆alkyl, a C₁₋₆alkylene-C₃₋₆cycloalkyl, an aryl, a C₁₋₃alkaryl, a heteroaryl, and a C₁₋₃alkheteroaryl;
X is CH₂, O, S, or NH and n is 0 or 1; and
A is selected from among —CR′═CR″—, —C≡C—, a C₃₋₆cycloalkylene, an arylene, and a heteroarylene,
   where R′ and R″ are independently selected from among hydrogen and a C₁₋₆alkyl; and
B is selected from among hydrogen, a C₁₋₆alkyl, a C₃₋₆cycloalkyl, an aryl, a heteroaryl, and a fused ring moiety.

In a 25th aspect of the second embodiment W is CH₂, CHR₄, or CHR₅CHR₆, Z is ˜T-A-Xₙ—B, and T is —(CH₂)ₚ—(C(O))_{q}— (p is 0, 1, 2, or 3 and q is 0 or 1), and the compound of formula II is represented by formula (II-5)

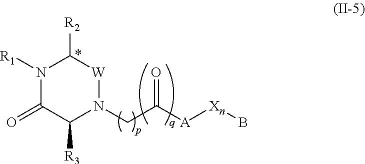

wherein
R₁ is hydrogen or a C₁₋₆alkyl;
R₂, R₃, R₄, R₅, and R₆ are independently selected from among hydrogen, a C₁₋₆alkyl, a C₃₋₆cycloalkyl, a C₁₋₆alkylene-C₃₋₆cycloalkyl, a C₁₋₆alkylenethioC₁₋₆alkyl, a C₁₋₆alkyleneoxoC₁₋₆alkyl, a C₁₋₆alkylene-C₃₋₆cycloalkyl, an aryl, a C₁₋₃alkaryl, a heteroaryl, and a C₁₋₃alkheteroaryl;
X is CH₂, O, S, or NH and n is 0 or 1; and
A is a heteroarylene; and
B is selected from among hydrogen, a C₁₋₆alkyl, a C₃₋₆cycloalkyl, an aryl, a heteroaryl, and a fused ring moiety.

In a 26th aspect of the second embodiment W is CH₂, CHR₄, or CHR₅CHR₆, Z is ˜T-A-Xₙ—B, and T is —(CH₂)ₚ—(C(O))_{q}— (p is 0, 1, 2, or 3 and q is 0 or 1), and the compound of formula II is represented by formula (II-5)

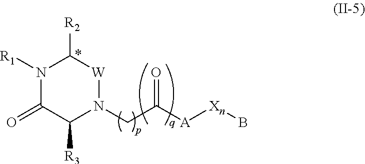

wherein
R₁ is hydrogen or a C₁₋₆alkyl;
R₂, R₃, R₄, R₅, and R₆ are independently selected from among hydrogen, a C₁₋₆alkyl, a C₃₋₆cycloalkyl, a C₁₋₆alkylene-C₃₋₆cycloalkyl, a C₁₋₆alkylenethioC₁₋₆alkyl, a C₁₋₆alkyleneoxoC₁₋₆alkyl, a C₁₋₆alkylene-C₃₋₆cycloalkyl, an aryl, a C₁₋₃alkaryl, a heteroaryl, and a C₁₋₃alkheteroaryl;
where n is 0 and B is bound to A and where;
A is selected from an N-ring, as defined herein; and
B is selected from among hydrogen, a C₁₋₆alkyl, a C₃₋₆cycloalkyl, an aryl, a heteroaryl, and a fused ring moiety.

In a 27th aspect of the second embodiment W is CH₂, CHR₄, or CHR₅CHR₆, Z is ˜T-A-Xₙ—B, and T is —(CH₂)ₚ—(C(O))_{q}— (p is 0, 1, 2, or 3 and q is 0 or 1), and the compound of formula II is represented by formula (II-5)

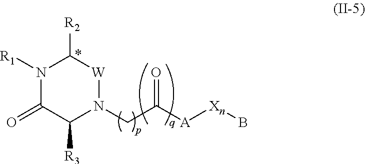

wherein
R₁ is hydrogen or a C₁₋₆alkyl;
R₂, R₃, R₄, R₅, and R₆ are independently selected from among hydrogen, a C₁₋₆alkyl, a C₃₋₆cycloalkyl, a C₁₋₆alkylene-C₃₋₆cycloalkyl, a C₁₋₆alkylenethioC₁₋₆alkyl, a C₁₋₆alkyleneoxoC₁₋₆alkyl, a C₁₋₆alkylene-C₃₋₆cycloalkyl, an aryl, a C₁₋₃alkaryl, a heteroaryl, and a C₁₋₃alkheteroaryl;
where n is 0 and B is bound to A and where;
A is selected from an A′-ring, as defined herein; and
B is selected from a B′-ring, as defined herein.

In a 28th aspect of the second embodiment W is CH₂, CHR₄, or CHR₅CHR₆, Z is ˜T-A-Xₙ—B, and T is —(CH₂)ₚ—(C(O))_{q}— (p is 0, 1, 2, or 3 and q is 0 or 1), and the compound of formula II is represented by formula (II-5)

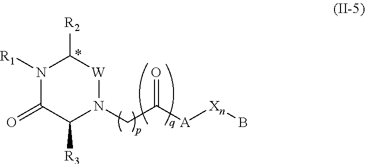

wherein
R₁ is hydrogen or a C₁₋₆alkyl;
R₂ is selected from among an R₂-substituent, as defined herein;
R₃ is selected from among CH₃, CH₂CH₃, CH₂CH₂CH₃, CH₂OCH₃, CH₂SCH₃, CH(CH₃)₂, CH(CH₃)(CH₂CH₃), C(CH₃)₃, CH₂CH(CH₃)₂, CH₂C(CH₃)₃, CH═CHCH₃, CH═CH, CH₂cPr, CH₂cBu, CH₂cPn, CH₂cHx, cPr, cBu, cPn, cHx, and CH₂Ph;
each of R₄, R₅, and R₆ is hydrogen;
where n is 0 and B is bound to A and where;
A is selected from an A′-ring, as defined herein; and
B is selected from a B′-ring, as defined herein.

In a sub-aspect of each of the 21st, 22nd, 23rd, 24th, 25th, 26th, 27th, and 28th aspects of the second embodiment, W is CH₂ and R₂ has a configuration as shown in formula (II-5′)

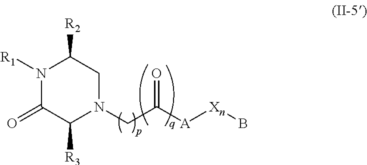

and R₃ is selected from among its respective listing in each of the above-mentioned aspects.

In a 29th aspect of the second embodiment W is CH₂, CHR₄, or CHR₅CHR₆, Z is ˜T-A-Xₙ—B, and T is —(CH₂)ₚ—(C(O))_{q}— (p is 0, 1, 2, or 3 and q is 0), and the compound of formula II is represented by formula (II-6)

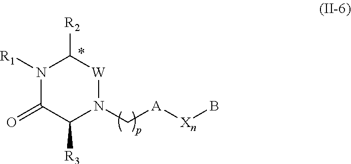

wherein
R₁ is selected from among hydrogen, an alkyl, an alkaryl, an acyl, an aryl, and a heteroaryl;
R₂, R₃, R₄, R₅, and R₆ are independently selected from among hydrogen, an alkyl, allyl, halo, a cycloalkyl, an alkylene-cycloalkyl, an alkylenethioalkyl, an alkyleneoxoalkyl, an alkylene-cycloalkyl, an aryl, an alkaryl, a heteroaryl, an alkheteroaryl, COOH, COOalkyl, CONHalkyl, and CON(alkyl)₂;
X is CH₂, O, S, or NH, where n is 0 or 1;
A is selected from among —CR′═CR″—, —C≡C—, a cycloalkylene, an arylene, and a heteroarylene,
   where R′ and R″ are independently selected from among hydrogen, a C₁₋₆alkyl, and an aryl; and
B is selected from among hydrogen, a C₁₋₆alkyl, a cycloalkyl, an aryl, a heteroaryl, and a fused ring moiety.

In an 30th aspect of the second embodiment W is CH₂, CHR₄, or CHR₅CHR₆, Z is ˜T-A-Xₙ—B, and T is —(CH₂)ₚ—(C(O))_{q}— (p is 0, 1, 2, or 3, and q is 0), and the compound of formula II is represented by formula (II-6)

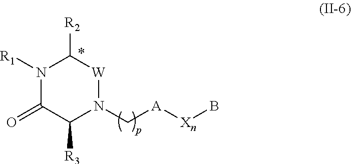

wherein
R₁ is selected from among hydrogen, an alkyl, an alkaryl, an acyl, an aryl, and a heteroaryl;
R₂, R₃, R₄, R₅, and R₆ are independently selected from among hydrogen, an alkyl, allyl, halo, a cycloalkyl, an alkylene-cycloalkyl, an alkylenethioalkyl, an alkyleneoxoalkyl, an alkylene-cycloalkyl, an aryl, an alkaryl, a heteroaryl, and an alkheteroaryl;
X is CH₂, O, S, or NH, where n is 0 or 1;
A is selected from among —CR′═CR″—, —C≡C—, a cycloalkylene, an arylene, and a heteroarylene,
   where R′ and R″ are independently selected from among hydrogen, a C₁₋₆alkyl, and an aryl; and
B is selected from among hydrogen, a C₁₋₆alkyl, a cycloalkyl, an aryl, a heteroaryl, and a fused ring moiety.

In a 31st aspect of the second embodiment W is CH₂, CHR₄, or CHR₅CHR₆, Z is ˜T-A-Xₙ—B, and T is —(CH₂)ₚ—(C(O))_{q}— (p is 0, 1, 2, or 3, and q is 0), and the compound of formula II is represented by formula (II-6)

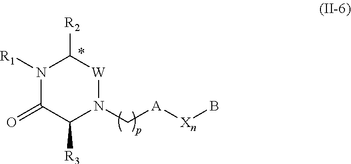

wherein
R₁ is selected from among hydrogen, a C₁₋₆alkyl and a C₁₋₃alkaryl;
R₂, R₃, R₄, R₅, and R₆ are independently selected from among hydrogen, a C₁₋₆alkyl, allyl, a C₃₋₆cycloalkyl, a C₁₋₆alkylene-C₃₋₆cycloalkyl, a C₁₋₆alkylenethioC₁₋₆alkyl, a C₁₋₆alkyleneoxoC₁₋₆alkyl, a C₁₋₆alkylene-C₃₋₆cycloalkyl, an aryl, a C₁₋₃alkaryl, a heteroaryl, and a C₁₋₃alkheteroaryl;
X is CH₂, O, S, or NH, where n is 0 or 1;
A is selected from among —CR′═CR″—, —C≡C—, a C₃₋₆cycloalkylene, an arylene, and a heteroarylene,
   where R′ and R″ are independently selected from among hydrogen, a C₁₋₆alkyl, and an aryl; and
B is selected from among hydrogen, a C₁₋₆alkyl, a C₃₋₆cycloalkyl, an aryl, a heteroaryl, and a fused ring moiety.

In a 32nd aspect of the second embodiment W is CH₂, CHR₄, or CHR₅CHR₆, Z is ˜T-A-Xₙ—B, and T is —(CH₂)ₚ—(C(O))_{q}— (p is 0, 1, 2, or 3, and q is 0), and the compound of formula II is represented by formula (II-6)

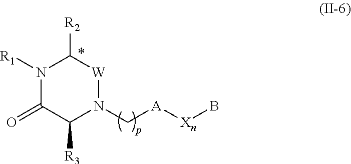

wherein
R₁ is hydrogen or a C₁₋₆alkyl;
R₂, R₃, R₄, R₅, and R₆ are independently selected from among hydrogen a C₁₋₆alkyl, a C₃₋₆cycloalkyl, a C₁₋₆alkylene-C₃₋₆cycloalkyl, a C₁₋₆alkylenethioC₁₋₆alkyl, a C₁₋₆alkyleneoxoC₁₋₆alkyl, a C₁₋₆alkylene-C₃₋₆cycloalkyl, an aryl, a C₁₋₃alkaryl, a heteroaryl, and a C₁₋₃alkheteroaryl;
X is CH₂, O, S, or NH, where n is 0 or 1;
A is selected from among —CR′═CR″—, —C≡C—, a C₃₋₆cycloalkylene, an arylene, and a heteroarylene,
   where R′ and R″ are independently selected from among hydrogen and a C₁₋₆alkyl; and
B is selected from among hydrogen, a C₁₋₆alkyl, a C₃₋₆cycloalkyl, an aryl, a heteroaryl, and a fused ring moiety.

In a 33rd aspect of the second embodiment W is CH₂, CHR₄, or CHR₅CHR₆, Z is ˜T-A-Xₙ—B, and T is —(CH₂)ₚ—(C(O))_{q}— (p is 0, 1, 2, or 3, and q is 0), and the compound of formula II is represented by formula (II-6)

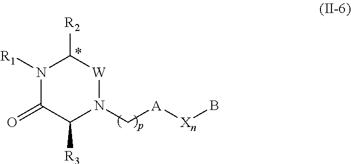

wherein
R₁ is hydrogen or a C₁₋₆alkyl;
R₂, R₃, R₄, R₅, and R₆ are independently selected from among hydrogen, a C₁₋₆alkyl, a C₃₋₆cycloalkyl, a C₁₋₆alkylene-C₃₋₆cycloalkyl, a C₁₋₆alkylenethioC₁₋₆alkyl, a C₁₋₆alkyleneoxoC₁₋₆alkyl, a C₁₋₆alkylene-C₃₋₆cycloalkyl, an aryl, a C₁₋₃alkaryl, a heteroaryl, and a C₁₋₃alkheteroaryl;
X is CH₂, O, S, or NH, where n is 0 or 1;
   A is a heteroarylene; and
B is selected from among hydrogen, a C₁₋₆alkyl, a C₃₋₆cycloalkyl, an aryl, a heteroaryl, and a fused ring moiety.

In a 34th aspect of the second embodiment W is CH₂, CHR₄, or CHR₅CHR₆, Z is ˜T-A-Xₙ—B, and T is —(CH₂)ₚ—(C(O))_{q}— (p is 0, 1, 2, or 3, and q is 0), and the compound of formula II is represented by formula (II-6)

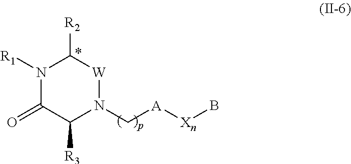

wherein
R₁ is hydrogen or a C₁₋₆alkyl;
R₂, R₃, R₄, R₅, and R₆ are independently selected from among a C₁₋₆alkyl, a C₃₋₆cycloalkyl, a C₁₋₆alkylene-C₃₋₆cycloalkyl, a C₁₋₆alkylenethioC₁₋₆alkyl, a C₁₋₆alkyleneoxoC₁₋₆alkyl, a C₁₋₆alkylene-C₃₋₆cycloalkyl, an aryl, a C₁₋₃alkaryl, a heteroaryl, and a C₁₋₃alkheteroaryl;
where n is 0 and B is bound to A and where;
A is selected from an A′-ring, as defined herein; and
B is selected from among hydrogen, a C₁₋₆alkyl, a C₃₋₆cycloalkyl, an aryl, a heteroaryl, and a fused ring moiety.

In a 35th aspect of the second embodiment W is CH₂, CHR₄, or CHR₅CHR₆, Z is ˜T-A-Xₙ—B, and T is —(CH₂)ₚ—(C(O))_{q}— (p is 0, 1, 2, or 3, and q is 0), and the compound of formula II is represented by formula (II-6)

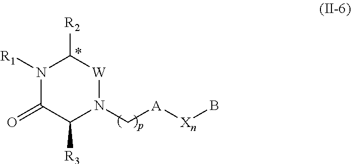

wherein
R₁ is hydrogen or a C₁₋₆alkyl;
R₂, R₃, R₄, R₅, and R₆ are independently selected from among hydrogen a C₁₋₆alkyl, a C₃₋₆cycloalkyl, a C₁₋₆alkylene-C₃₋₆cycloalkyl, a C₁₋₆alkylenethioC₁₋₆alkyl, a C₁₋₆alkyleneoxoC₁₋₆alkyl, a C₁₋₆alkylene-C₃₋₆cycloalkyl, an aryl, a C₁₋₃alkaryl, a heteroaryl, and a C₁₋₃alkheteroaryl;
where n is 0 and B is bound to A and where;
A is selected from an A′-ring, as defined herein; and
B is selected from a B′-ring, as defined herein.

In a 36th aspect of the second embodiment W is CH₂, CHR₄, or CHR₅CHR₆, Z is ˜T-A-Xₙ—B, and T is —(CH₂)ₚ—(C(O))_{q}— (p is 0, 1, 2, or 3, and q is 0), and the compound of formula II is represented by formula (II-6)

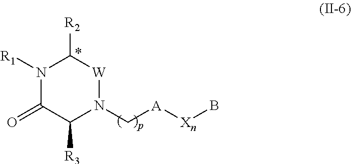

wherein
R₁ is hydrogen or a C₁₋₆alkyl;
R₂ is selected from among an R₂-substituent, as defined herein;
R₃ is selected from among CH₃, CH₂CH₃, CH₂CH₂CH₃, CH₂OCH₃, CH₂SCH₃, CH(CH₃)₂, CH(CH₃)(CH₂CH₃), C(CH₃)₃, CH₂CH(CH₃)₂, CH₂C(CH₃)₃, CH═CHCH₃, CH═CH, CH₂cPr, CH₂cBu, CH₂cPn, CH₂cHx, cPr, cBu, cPn, cHx, and CH₂Ph;
each of R₄, R₅, and R₆ is hydrogen;
where n is 0 and B is bound to A and where;
A is selected from an A′-ring, as defined herein; and
B is selected from a B′-ring, as defined herein.

In a sub-aspect of each of the 29th, 30th, 31st, 32nd, 33rd, 34th, 35th, and 36th aspects of the second embodiment, W is CH₂ and R₂ has a configuration as shown in formula (II-6′)

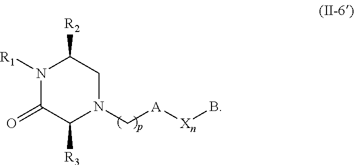

and R₃ is selected from among its respective listing in each of the above-mentioned aspects.

In a 37th aspect of the second embodiment W is CH₂, CHR₄, or CHR₅CHR₆, Z is ˜T-A-Xₙ—B, and T is —(CH₂)ₚ—(C(O))_{q}— (p is 0, 1, 2, or 3, q is 0, and n is 0), and the compound of formula II is represented by formula (II-7)

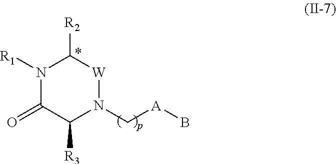

wherein
R₁ is selected from among hydrogen, an alkyl, an alkaryl, an acyl, an aryl, and a heteroaryl;
R₂, R₃, R₄, R₅, and R₆ are independently selected from among hydrogen, an alkyl, allyl, halo, a cycloalkyl, an alkylene-cycloalkyl, an alkylenethioalkyl, an alkyleneoxoalkyl, an alkylene-cycloalkyl, an aryl, an alkaryl, a heteroaryl, an alkheteroaryl, COOH, COOalkyl, CONHalkyl, and CON(alkyl)₂;
A is selected from among —CR′═CR″—, —C≡C—, a cycloalkylene, an arylene, and a heteroarylene,
   where R′ and R″ are independently selected from among hydrogen, a C₁₋₆alkyl, and an aryl; and
B is selected from among hydrogen, a C₁₋₆alkyl, a cycloalkyl, an aryl, a heteroaryl, and a fused ring moiety.

In an 38th aspect of the second embodiment W is CH₂, CHR₄, or CHR₅CHR₆, Z is ˜T-A-Xₙ—B, and T is —(CH₂)ₚ—(C(O))_{q}— (p is 0, 1, 2, or 3, q is 0, and n is 0), and the compound of formula II is represented by formula (II-7)

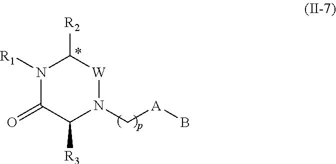

wherein
R₁ is selected from among hydrogen, an alkyl, an alkaryl, an acyl, an aryl, and a heteroaryl;
R₂, R₃, R₄, R₅, and R₆ are independently selected from among hydrogen, an alkyl, allyl, halo, a cycloalkyl, an alkylene-cycloalkyl, an alkylenethioalkyl, an alkyleneoxoalkyl, an alkylene-cycloalkyl, an aryl, an alkaryl, a heteroaryl, and an alkheteroaryl;
A is selected from among —CR′═CR″—, —C≡C—, a cycloalkylene, an arylene, and a heteroarylene,
   where R′ and R″ are independently selected from among hydrogen, a C₁₋₆alkyl, and an aryl; and
B is selected from among hydrogen, a C₁₋₆alkyl, a cycloalkyl, an aryl, a heteroaryl, and a fused ring moiety.

In a 39th aspect of the second embodiment W is CH₂, CHR₄, or CHR₅CHR₆, Z is ˜T-A-Xₙ—B, and T is —(CH₂)ₚ—(C(O))_{q}— (p is 0, 1, 2, or 3, q is 0, and n is 0), and the compound of formula II is represented by formula (II-7)

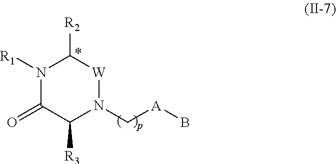

wherein
R₁ is selected from among hydrogen, a C₁₋₆alkyl and a C₁₋₃alkaryl;
R₂, R₃, R₄, R₅, and R₆ are independently selected from among hydrogen, a C₁₋₆alkyl, allyl, a C₃₋₆cycloalkyl, a C₁₋₆alkylene-C₃₋₆cycloalkyl, a C₁₋₆alkylenethioC₁₋₆alkyl, an C₁₋₆alkyleneoxoC₁₋₆alkyl, a C₁₋₆alkylene-C₃₋₆cycloalkyl, an aryl, an C₁₋₃alkaryl, a heteroaryl, and a C₁₋₃alkheteroaryl;
A is selected from among —CR′═CR″—, —C≡C—, a C₃₋₆cycloalkylene, an arylene, and a heteroarylene,
   where R′ and R″ are independently selected from among hydrogen, a C₁₋₆alkyl, and an aryl; and
B is selected from among hydrogen, a C₁₋₆alkyl, a C₃₋₆cycloalkyl, an aryl, a heteroaryl, and a fused ring moiety.

In a 40th aspect of the second embodiment W is CH₂, CHR₄, or CHR₅CHR₆, Z is ˜T-A-Xₙ—B, and T is —(CH₂)ₚ—(C(O))_{q}— (p is 0, 1, 2, or 3, q is 0, and n is 0), and the compound of formula II is represented by formula (II-7)

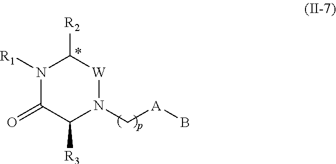

wherein
R₁ is hydrogen or a C₁₋₆alkyl;
R₂, R₃, R₄, R₅, and R₆ are independently selected from among hydrogen, a C₁₋₆alkyl, a C₃₋₆cycloalkyl, a C₁₋₆alkylene-C₃₋₆cycloalkyl, a C₁₋₆alkylenethioC₁₋₆alkyl, a C₁₋₆alkyleneoxoC₁₋₆alkyl, a C₁₋₆alkylene-C₃₋₆cycloalkyl, an aryl, a C₁₋₃alkaryl, a heteroaryl, and a C₁₋₃alkheteroaryl;
A is selected from among —CR′═CR″—, —C≡C—, a C₃₋₆cycloalkylene, an arylene, and a heteroarylene,
   where R′ and R″ are independently selected from among hydrogen and a C₁₋₆alkyl; and
B is selected from among hydrogen, a C₁₋₆alkyl, a C₃₋₆cycloalkyl, an aryl, a heteroaryl, and a fused ring moiety.

In a 41st aspect of the second embodiment W is CH₂, CHR₄, or CHR₅CHR₆, Z is ˜T-A-Xₙ—B, and T is —(CH₂)ₚ—(C(O))_{q}— (p is 0, 1, 2, or 3, q is 0, and n is 0), and the compound of formula II is represented by formula (II-7)

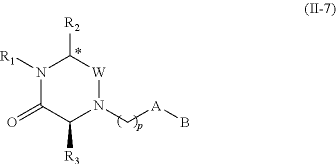

wherein
R₁ is hydrogen or a C₁₋₆alkyl;
R₂, R₃, R₄, R₅, and R₆ are independently selected from among hydrogen, a C₁₋₆alkyl, a C₃₋₆cycloalkyl, a C₁₋₆alkylene-C₃₋₆cycloalkyl, a C₁₋₆alkylenethioC₁₋₆alkyl, a C₁₋₆alkyleneoxoC₁₋₆alkyl, a C₁₋₆alkylene-C₃₋₆cycloalkyl, an aryl, a C₁₋₃alkaryl, a heteroaryl, and a C₁₋₃alkheteroaryl;
A is a heteroarylene; and
B is selected from among hydrogen, a C₁₋₆alkyl, a C₃₋₆cycloalkyl, an aryl, a heteroaryl, and a fused ring moiety.

In a 42nd aspect of the second embodiment W is CH₂, CHR₄, or CHR₅CHR₆, Z is ˜T-A-Xₙ—B, and T is —(CH₂)ₚ—(C(O))_{q}— (p is 0, 1, 2, or 3, q is 0, and n is 0), and the compound of formula II is represented by formula (II-7)

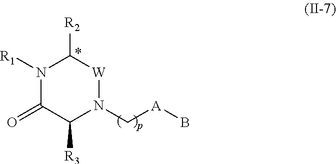

wherein
R₁ is hydrogen or a C₁₋₆alkyl;
R₂, R₃, R₄, R₅, and R₆ are independently selected from among hydrogen, a C₁₋₆alkyl, a C₃₋₆cycloalkyl, a C₁₋₆alkylene-C₃₋₆cycloalkyl, a C₁₋₆alkylenethioC₁₋₆alkyl, a C₁₋₆alkyleneoxoC₁₋₆alkyl, a C₁₋₆alkylene-C₃₋₆cycloalkyl, an aryl, a C₁₋₃alkaryl, a heteroaryl, and a C₁₋₃alkheteroaryl;
where n is 0 and B is bound to A and where;
A is selected from an N-ring, as defined herein; and
B is selected from among hydrogen, a C₁₋₆alkyl, a C₃₋₆cycloalkyl, an aryl, a heteroaryl, and a fused ring moiety.

In a 43rd aspect of the second embodiment W is CH₂, CHR₄, or CHR₅CHR₆, Z is ˜T-A-Xₙ—B, T is —(CH₂)ₚ—(C(O))_{q}— (p is 0, 1, 2, or 3, q is 0, and n is 0) and the compound of formula II is represented by formula (II-7)

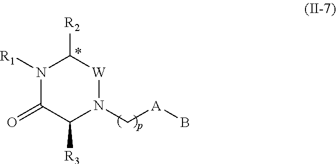

wherein
R₁ is hydrogen or a C₁₋₆alkyl;
R₂, R₃, R₄, R₅, and R₆ are independently selected from among hydrogen, a C₁₋₆alkyl, a C₃₋₆cycloalkyl, a C₁₋₆alkylene-C₃₋₆cycloalkyl, a C₁₋₆alkylenethioC₁₋₆alkyl, a C₁₋₆alkyleneoxoC₁₋₆alkyl, a C₁₋₆alkylene-C₃₋₆cycloalkyl, an aryl, a C₁₋₃alkaryl, a heteroaryl, and a C₁₋₃alkheteroaryl;
where n is 0 and B is bound to A and where;
A is selected from an A′-ring, as defined herein; and
B is selected from a B′-ring, as defined herein.

In a 44th aspect of the second embodiment W is CH₂, CHR₄, or CHR₅CHR₆, Z is ˜T-A-Xₙ—B, T is —(CH₂)ₚ—(C(O))_{q}— (p is 0, 1, 2, or 3, q is 0, and n is 0) and the compound of formula II is represented by formula (II-7)

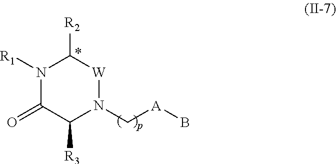

wherein
R₁ is hydrogen;
R₂ is selected from among an R₂-substituent, as defined herein;
R₃ is selected from among CH₃, CH₂CH₃, CH₂CH₂CH₃, CH₂OCH₃, CH₂SCH₃, CH(CH₃)₂, CH(CH₃)(CH₂CH₃), C(CH₃)₃, CH₂CH(CH₃)₂, CH₂C(CH₃)₃, CH═CHCH₃, CH═CH, CH₂cPr, CH₂cBu, CH₂cPn, CH₂cHx, cPr, cBu, cPn, cHx, and CH₂Ph;
each of R₄, R₅, and R₆ is hydrogen;
A is selected from an A′-ring, as defined herein; and
B is selected from a B′-ring, as defined herein.

In a sub-aspect of each of the 37th, 38th, 39th, 40th, 41st, 42nd, 43rd, and 44th aspects of the second embodiment, W is CH₂ and R₂ has a configuration as shown in formula (II-7′)

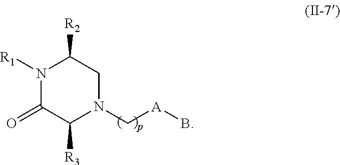

and R₃ is selected from among its respective listing in each of the above-mentioned aspects.

In a 45th aspect of the second embodiment W is CH₂, CHR₄, or CHR₅CHR₆, Z is ˜T-A-Xₙ—B, and T is —(CH₂)ₚ—(C(O))_{q}— (p is 0, 1, 2, or 3 and q is 1), and the compound of formula II is represented by formula (II-8)

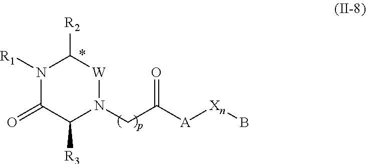

wherein
R₁ is selected from among hydrogen, an alkyl, an alkaryl, an acyl, an aryl, and a heteroaryl;
R₂, R₃, R₄, R₅, and R₆ are independently selected from among hydrogen, an alkyl, allyl, halo, a cycloalkyl, an alkylene-cycloalkyl, an alkylenethioalkyl, an alkyleneoxoalkyl, an alkylene-cycloalkyl, an aryl, an alkaryl, a heteroaryl, an alkheteroaryl, COOH, COOalkyl, CONHalkyl, and CON(alkyl)₂;
X is CH₂, O, S, or NH, where n is 0 or 1;
A is selected from among —CR′═CR″—, —C≡C—, a cycloalkylene, an arylene, and a heteroarylene,
   where R′ and R″ are independently selected from among hydrogen, a C₁₋₆alkyl, and an aryl; and
B is selected from among hydrogen, a C₁₋₆alkyl, a cycloalkyl, an aryl, a heteroaryl, and a fused ring moiety.

In an 46th aspect of the second embodiment W is CH₂, CHR₄, or CHR₅CHR₆, Z is ˜T-A-Xₙ—B, and T is —(CH₂)ₚ—(C(O))_{q}— (p is 0, 1, 2, or 3 and q is 1), and the compound of formula II is represented by formula (II-8)

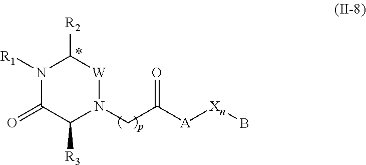

wherein
R₁ is selected from among hydrogen, an alkyl, an alkaryl, an acyl, an aryl, and a heteroaryl;
R₂, R₃, R₄, R₅, and R₆ are independently selected from among hydrogen, an alkyl, allyl, halo, a cycloalkyl, an alkylene-cycloalkyl, an alkylenethioalkyl, an alkyleneoxoalkyl, an alkylene-cycloalkyl, an aryl, an alkaryl, a heteroaryl, and an alkheteroaryl;
X is CH₂, O, S, or NH, where n is 0 or 1;
A is selected from among —CR′═CR″—, —C≡C—, a cycloalkylene, an arylene, and a heteroarylene,
   where R′ and R″ are independently selected from among hydrogen, a C₁₋₆alkyl, and an aryl; and
B is selected from among hydrogen, a C₁₋₆alkyl, a cycloalkyl, an aryl, a heteroaryl, and a fused ring moiety.

In a 47th aspect of the second embodiment W is CH₂, CHR₄, or CHR₅CHR₆, Z is ˜T-A-Xₙ—B, and T is —(CH₂)ₚ—(C(O))_{q}— (p is 0, 1, 2, or 3 and q is 1), and the compound of formula II is represented by formula (II-8)

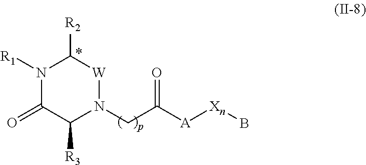

wherein
R₁ is selected from among hydrogen, a C₁₋₆alkyl and a C₁₋₃alkaryl;
R₂, R₃, R₄, R₅, and R₆ are independently selected from among hydrogen, a C₁₋₆alkyl, allyl, a C₃₋₆cycloalkyl, a C₁₋₆alkylene-C₃₋₆cycloalkyl, a C₁₋₆alkylenethioC₁₋₆alkyl, a C₁₋₆alkyleneoxoC₁₋₆alkyl, a C₁₋₆alkylene-C₃₋₆cycloalkyl, an aryl, a C₁₋₃alkaryl, a heteroaryl, and a C₁₋₃alkheteroaryl;
X is CH₂, O, S, or NH, where n is 0 or 1;
A is selected from among —CR′═CR″—, —C≡C—, a C₃₋₆cycloalkylene, an arylene, and a heteroarylene,
   where R′ and R″ are independently selected from among hydrogen, a C₁₋₆alkyl, and an aryl; and
B is selected from among hydrogen, a C₁₋₆alkyl, a C₃₋₆cycloalkyl, an aryl, a heteroaryl, and a fused ring moiety.

In a 48th aspect of the second embodiment W is CH₂, CHR₄, or CHR₅CHR₆, Z is ˜T-A-Xₙ—B, and T is —(CH₂)ₚ—(C(O))_{q}— (p is 0, 1, 2, or 3 and q is 1), and the compound of formula II is represented by formula (II-8)

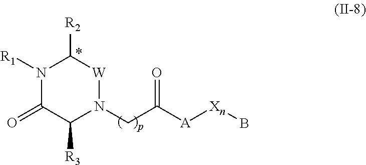

wherein
R₁ is hydrogen or a C₁₋₆alkyl;
R₂, R₃, R₄, R₅, and R₆ are independently selected from among hydrogen, a C₁₋₆alkyl, a C₃₋₆cycloalkyl, a C₁₋₆alkylene-C₃₋₆cycloalkyl, a C₁₋₆alkylenethioC₁₋₆alkyl, an C₁₋₆alkyleneoxoC₁₋₆alkyl, a C₁₋₆alkylene-C₃₋₆cycloalkyl, an aryl, an C₁₋₃alkaryl, a heteroaryl, and a C₁₋₃alkheteroaryl;
X is CH₂, O, S, or NH, where n is 0 or 1;
A is selected from among —CR′═CR″—, —C≡C—, a C₃₋₆cycloalkylene, an arylene, and a heteroarylene,
   where R′ and R″ are independently selected from among hydrogen and a C₁₋₆alkyl; and
B is selected from among hydrogen, a C₁₋₆alkyl, a C₃₋₆cycloalkyl, an aryl, a heteroaryl, and a fused ring moiety.

In a 49th aspect of the second embodiment W is CH₂, CHR₄, or CHR₅CHR₆, Z is ˜T-A-Xₙ—B, and T is —(CH₂)ₚ—(C(O))_{q}— (p is 0, 1, 2, or 3 and q is 1), and the compound of formula II is represented by formula (II-8)

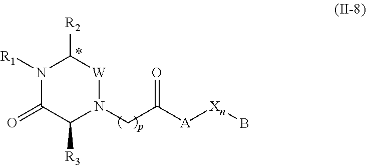

wherein
R₁ is hydrogen or a C₁₋₆alkyl;
R₂, R₃, R₄, R₅, and R₆ are independently selected from among hydrogen, a C₁₋₆alkyl, a C₃₋₆cycloalkyl, a C₁₋₆alkylene-C₃₋₆cycloalkyl, a C₁₋₆alkylenethioC₁₋₆alkyl, a C₁₋₆alkyleneoxoC₁₋₆alkyl, a C₁₋₆alkylene-C₃₋₆cycloalkyl, an aryl, an C₁₋₃alkaryl, a heteroaryl, and a C₁₋₃alkheteroaryl;
X is CH₂, O, S, or NH, where n is 0 or 1;
   A is a heteroarylene; and
B is selected from among hydrogen, a C₁₋₆alkyl, a C₃₋₆cycloalkyl, an aryl, a heteroaryl, and a fused ring moiety.

In a 50th aspect of the second embodiment W is CH₂, CHR₄, or CHR₅CHR₆, Z is ˜T-A-Xₙ—B, and T is —(CH₂)ₚ—(C(O))_{q}— (p is 0, 1, 2, or 3 and q is 1), and the compound of formula II is represented by formula (II-8)

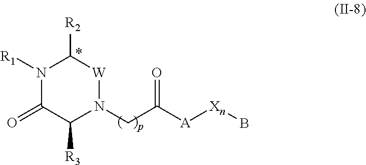

wherein
R₁ is hydrogen or a C₁₋₆alkyl;
R₂, R₃, R₄, R₅, and R₆ are independently selected from among hydrogen, a C₁₋₆alkyl, a C₃₋₆cycloalkyl, a C₁₋₆alkylene-C₃₋₆cycloalkyl, a C₁₋₆alkylenethioC₁₋₆alkyl, a C₁₋₆alkyleneoxoC₁₋₆alkyl, a C₁₋₆alkylene-C₃₋₆cycloalkyl, an aryl, a C₁₋₃alkaryl, a heteroaryl, and a C₁₋₃alkheteroaryl;
where n is 0 and B is bound to A and where;
A is selected from an N-ring, as defined herein; and
B is selected from among hydrogen, a C₁₋₆alkyl, a C₃₋₆cycloalkyl, an aryl, a heteroaryl, and a fused ring moiety.

In a 51st aspect of the second embodiment W is CH₂, CHR₄, or CHR₅CHR₆, Z is ˜T-A-Xₙ—B, and T is —(CH₂)ₚ—(C(O))_{q}— (p is 0, 1, 2, or 3 and q is 1), and the compound of formula II is represented by formula (II-8)

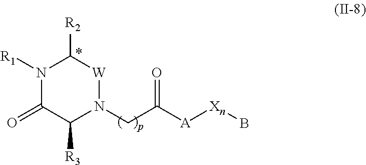

wherein
R₁ is hydrogen or a C₁₋₆alkyl;
R₂, R₃, R₄, R₅, and R₆ are independently selected from among hydrogen, a C₁₋₆alkyl, a C₃₋₆cycloalkyl, a C₁₋₆alkylene-C₃₋₆cycloalkyl, a C₁₋₆alkylenethioC₁₋₆alkyl, a C₁₋₆alkyleneoxoC₁₋₆alkyl, a C₁₋₆alkylene-C₃₋₆cycloalkyl, an aryl, an C₁₋₃alkaryl, a heteroaryl, and a C₁₋₃alkheteroaryl;
where n is 0 and B is bound to A and where;
A is selected from an A′-ring, as defined herein; and
B is selected from a B′-ring, as defined herein.

In a 52nd aspect of the second embodiment W is CH₂, CHR₄, or CHR₅CHR₆, Z is ˜T-A-Xₙ—B, and T is —(CH₂)ₚ—(C(O))_{q}— (p is 0, 1, 2, or 3 and q is 1), and the compound of formula II is represented by formula (II-8)

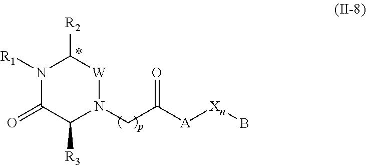

wherein
R₁ is hydrogen;
R₂ is selected from among an R₂-substituent, as defined herein;
R₃ is selected from among CH₃, CH₂CH₃, CH₂CH₂CH₃, CH₂OCH₃, CH₂SCH₃, CH(CH₃)₂, CH(CH₃)(CH₂CH₃), C(CH₃)₃, CH₂CH(CH₃)₂, CH₂C(CH₃)₃, CH═CHCH₃, CH═CH, CH₂cPr, CH₂cBu, CH₂cPn, CH₂cHx, cPr, cBu, cPn, cHx, and CH₂Ph;
each of R₄, R₅, and R₆ is hydrogen;
where n is 0 and B is bound to A and where;
A is selected from an A′-ring, as defined herein; and
B is selected from a B′-ring, as defined herein.

In a sub-aspect of each of the 45th, 46th, 47th, 48th, 49th, 50th, 51st, and 52nd aspects of the second embodiment, W is CH₂ and R₂ has a configuration as shown in formula (II-8′)

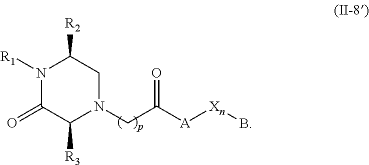

and R₃ is selected from among its respective listing in each of the above-mentioned aspects.

In a 53rd aspect of the second embodiment W is CH₂, CHR₄, or CHR₅CHR₆, Z is ˜T-A-Xₙ—B, T is —(CH₂)ₚ—(C(O))_{q}— (p is 0, 1, 2, or 3 and q is 1), and n is 0, and the compound of formula II is represented by formula (II-9)

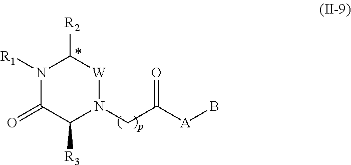

wherein
R₁ is selected from among hydrogen, an alkyl, an alkaryl, an acyl, an aryl, and a heteroaryl;
R₂, R₃, R₄, R₅, and R₆ are independently selected from among hydrogen, an alkyl, allyl, halo, a cycloalkyl, an alkylene-cycloalkyl, an alkylenethioalkyl, an alkyleneoxoalkyl, an alkylene-cycloalkyl, an aryl, an alkaryl, a heteroaryl, an alkheteroaryl, COOH, COOalkyl, CONHalkyl, and CON(alkyl)₂;
A is selected from among —CR′═CR″—, —C≡C—, a cycloalkylene, an arylene, and a heteroarylene,
   where R′ and R″ are independently selected from among hydrogen, a C₁₋₆alkyl, and an aryl; and
B is selected from among hydrogen, a C₁₋₆alkyl, a cycloalkyl, an aryl, a heteroaryl, and a fused ring moiety.

In an 54th aspect of the second embodiment W is CH₂, CHR₄, or CHR₅CHR₆, Z is ˜T-A-Xₙ—B, T is —(CH₂)ₚ—(C(O))_{q}— (p is 0, 1, 2, or 3 and q is 1), and n is 0, and the compound of formula II is represented by formula (II-9)

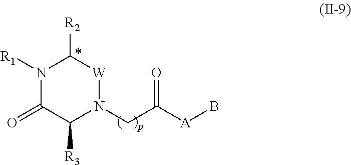

wherein
R₁ is selected from among hydrogen, an alkyl, an alkaryl, an acyl, an aryl, and a heteroaryl;
R₂, R₃, R₄, R₅, and R₆ are independently selected from among hydrogen, an alkyl, allyl, halo, a cycloalkyl, an alkylene-cycloalkyl, an alkylenethioalkyl, an alkyleneoxoalkyl, an alkylene-cycloalkyl, an aryl, an alkaryl, a heteroaryl, and an alkheteroaryl;
A is selected from among —CR′═CR″—, —C≡C—, a cycloalkylene, an arylene, and a heteroarylene,
   where R′ and R″ are independently selected from among hydrogen, a C₁₋₆alkyl, and an aryl; and
B is selected from among hydrogen, a C₁₋₆alkyl, a cycloalkyl, an aryl, a heteroaryl, and a fused ring moiety.

In a 55th aspect of the second embodiment W is CH₂, CHR₄, or CHR₅CHR₆, Z is ˜T-A-Xₙ—B, T is —(CH₂)ₚ—(C(O))_{q}— (p is 0, 1, 2, or 3 and q is 1), and n is 0, and the compound of formula II is represented by formula (II-9)

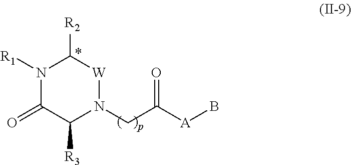

wherein
R₁ is selected from among hydrogen, a C₁₋₆alkyl and a C₁₋₃alkaryl;
R₂, R₃, R₄, R₅, and R₆ are independently selected from among hydrogen, a C₁₋₆alkyl, allyl, a C₃₋₆cycloalkyl, a C₁₋₆alkylene-C₃₋₆cycloalkyl, a C₁₋₆alkylenethioC₁₋₆alkyl, an C₁₋₆alkyleneoxoC₁₋₆alkyl, a C₁₋₆alkylene-C₃₋₆cycloalkyl, an aryl, an C₁₋₃alkaryl, a heteroaryl, and a C₁₋₃alkheteroaryl;
A is selected from among —CR′═CR″—, —C≡C—, a C₃₋₆cycloalkylene, an arylene, and a heteroarylene,
   where R′ and R″ are independently selected from among hydrogen, a C₁₋₆alkyl, and an aryl; and
B is selected from among hydrogen, a C₁₋₆alkyl, a C₃₋₆cycloalkyl, an aryl, a heteroaryl, and a fused ring moiety.

In a 56th aspect of the second embodiment W is CH₂, CHR₄, or CHR₅CHR₆, Z is ˜T-A-Xₙ—B, T is —(CH₂)ₚ—(C(O))_{q}— (p is 0, 1, 2, or 3 and q is 1), and n is 0, and the compound of formula II is represented by formula (II-9)

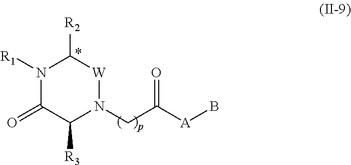

wherein
R₁ is hydrogen or a C₁₋₆alkyl;
R₂, R₃, R₄, R₅, and R₆ are independently selected from among hydrogen, a C₁₋₆alkyl, a C₃₋₆cycloalkyl, a C₁₋₆alkylene-C₃₋₆cycloalkyl, a C₁₋₆alkylenethioC₁₋₆alkyl, a C₁₋₆alkyleneoxoC₁₋₆alkyl, a C₁₋₆alkylene-C₃₋₆cycloalkyl, an aryl, a C₁₋₃alkaryl, a heteroaryl, and a C₁₋₃alkheteroaryl;
A is selected from among —CR′═CR″—, —C≡C—, a C₃₋₆cycloalkylene, an arylene, and a heteroarylene,
   where R′ and R″ are independently selected from among hydrogen and a C₁₋₆alkyl; and
B is selected from among hydrogen, a C₁₋₆alkyl, a C₃₋₆cycloalkyl, an aryl, a heteroaryl, and a fused ring moiety.

In a 57th aspect of the second embodiment W is CH₂, CHR₄, or CHR₅CHR₆, Z is ˜T-A-Xₙ—B, T is —(CH₂)ₚ—(C(O))_{q}— (p is 0, 1, 2, or 3 and q is 1), and n is 0, and the compound of formula II is represented by formula (II-9)

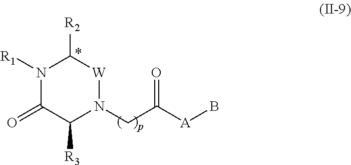

wherein
R₁ is hydrogen or a C₁₋₆alkyl;
R₂, R₃, R₄, R₅, and R₆ are independently selected from among hydrogen, a C₁₋₆alkyl, a C₃₋₆cycloalkyl, a C₁₋₆alkylene-C₃₋₆cycloalkyl, a C₁₋₆alkylenethioC₁₋₆alkyl, a C₁₋₆alkyleneoxoC₁₋₆alkyl, a C₁₋₆alkylene-C₃₋₆cycloalkyl, an aryl, a C₁₋₃alkaryl, a heteroaryl, and a C₁₋₃alkheteroaryl;
A is a heteroarylene; and
B is selected from among hydrogen, a C₁₋₆alkyl, a C₃₋₆cycloalkyl, an aryl, a heteroaryl, and a fused ring moiety.

In a 58th aspect of the second embodiment W is CH₂, CHR₄, or CHR₅CHR₆, Z is ˜T-A-Xₙ—B, T is —(CH₂)ₚ—(C(O))_{q}— (p is 0, 1, 2, or 3 and q is 1), and n is 0, and the compound of formula II is represented by formula (II-9)

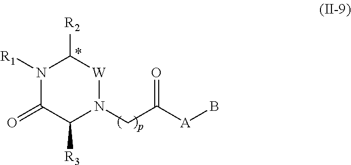

wherein
R₁ is hydrogen or a C₁₋₆alkyl;
R₂, R₃, R₄, R₅, and R₆ are independently selected from among hydrogen, a C₁₋₆alkyl, a C₃₋₆cycloalkyl, a C₁₋₆alkylene-C₃₋₆cycloalkyl, a C₁₋₆alkylenethioC₁₋₆alkyl, a C₁₋₆alkyleneoxoC₁₋₆alkyl, a C₁₋₆alkylene-C₃₋₆cycloalkyl, an aryl, an C₁₋₃alkaryl, a heteroaryl, and a C₁₋₃alkheteroaryl;
A is selected from an A′-ring, as defined herein; and
B is selected from among hydrogen, a C₁₋₆alkyl, a C₃₋₆cycloalkyl, an aryl, a heteroaryl, and a fused ring moiety.

In a 59th aspect of the second embodiment W is CH₂, CHR₄, or CHR₅CHR₆, Z is ˜T-A-Xₙ—B, T is —(CH₂)ₚ—(C(O))_{q}— (p is 0, 1, 2, or 3 and q is 1), and n is 0, and the compound of formula II is represented by formula (II-9)

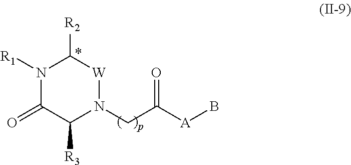

wherein
R₁ is hydrogen or a C₁₋₆alkyl;
R₂, R₃, R₄, R₅, and R₆ are independently selected from among hydrogen, a C₁₋₆alkyl, a C₃₋₆cycloalkyl, a C₁₋₆alkylene-C₃₋₆cycloalkyl, a C₁₋₆alkylenethioC₁₋₆alkyl, a C₁₋₆alkyleneoxoC₁₋₆alkyl, a C₁₋₆alkylene-C₃₋₆cycloalkyl, an aryl, a C₁₋₃alkaryl, a heteroaryl, and a C₁₋₃alkheteroaryl;
A is selected from an A′-ring, as defined herein; and
B is selected from a B′-ring, as defined herein.

In a 60th aspect of the second embodiment W is CH₂, CHR₄, or CHR₅CHR₆, Z is ˜T-A-Xₙ—B, T is —(CH₂)ₚ—(C(O))_{q}— (p is 0, 1, 2, or 3 and q is 1), and n is 0, and the compound of formula II is represented by formula (II-9)

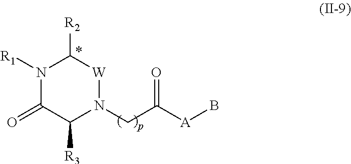

wherein
R₁ is hydrogen;
R₂ is selected from among an R₂-substituent, as defined herein;
R₃ is selected from among CH₃, CH₂CH₃, CH₂CH₂CH₃, CH₂OCH₃, CH₂SCH₃, CH(CH₃)₂, CH(CH₃)(CH₂CH₃), C(CH₃)₃, CH₂CH(CH₃)₂, CH₂C(CH₃)₃, CH═CHCH₃, CH═CH, CH₂cPr, CH₂cBu, CH₂cPn, CH₂cHx, cPr, cBu, cPn, cHx, and CH₂Ph;
each of R₄, R₅, and R₆ is hydrogen;
A is selected from an A′-ring, as defined herein; and
B is selected from a B′-ring, as defined herein.

In a sub-aspect of each of the 53rd, 54th, 55th, 56th, 57th, 58th, 59th, and 60th aspects of the second embodiment, W is CH₂ and R₂ has a configuration as shown in formula (II-9′)

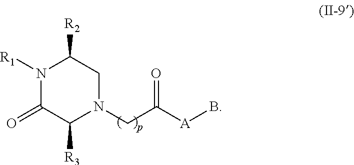

and R₃ is selected from among its respective listing in each of the above-mentioned aspects.

In a 61st aspect of the second embodiment W is CH₂, CHR₄, or CHR₅CHR₆, Z is ˜T-A-Xₙ—B, and T is —(CH₂)ₚ—(C(O))_{q}— (p is 0 and q is 1), and the compound of formula II is represented by formula (II-10)

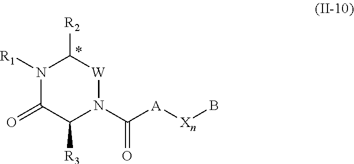

wherein
R₁ is selected from among hydrogen, an alkyl, an alkaryl, an acyl, an aryl, and a heteroaryl;
R₂, R₃, R₄, R₅, and R₆ are independently selected from among hydrogen, an alkyl, allyl, halo, a cycloalkyl, an alkylene-cycloalkyl, an alkylenethioalkyl, an alkyleneoxoalkyl, an alkylene-cycloalkyl, an aryl, an alkaryl, a heteroaryl, an alkheteroaryl, COOH, COOalkyl, CONHalkyl, and CON(alkyl)₂;
X is CH₂, O, S, or NH, where n is 0 or 1;
A is selected from among —CR′═CR″—, —C≡C—, a cycloalkylene, an arylene, and a heteroarylene,
   where R′ and R″ are independently selected from among hydrogen, a C₁₋₆alkyl, and an aryl; and
B is selected from among hydrogen, a C₁₋₆alkyl, a cycloalkyl, an aryl, a heteroaryl, and a fused ring moiety.

In an 62nd aspect of the second embodiment W is CH₂, CHR₄, or CHR₅CHR₆, Z is ˜T-A-Xₙ—B, and T is —(CH₂)ₚ—(C(O))_{q}— (p is 0 and q is 1), and the compound of formula II is represented by formula (II-10)

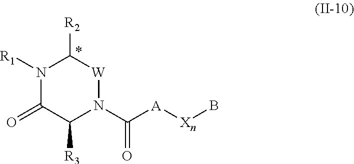

wherein
R₁ is selected from among hydrogen, an alkyl, an alkaryl, an acyl, an aryl, and a heteroaryl;
R₂, R₃, R₄, R₅, and R₆ are independently selected from among hydrogen, an alkyl, allyl, halo, a cycloalkyl, an alkylene-cycloalkyl, an alkylenethioalkyl, an alkyleneoxoalkyl, an alkylene-cycloalkyl, an aryl, an alkaryl, a heteroaryl, and an alkheteroaryl;
X is CH₂, O, S, or NH, where n is 0 or 1;
A is selected from among —CR′═CR″—, —C≡C—, a cycloalkylene, an arylene, and a heteroarylene,
   where R′ and R″ are independently selected from among hydrogen, a C₁₋₆alkyl, and an aryl; and
B is selected from among hydrogen, a C₁₋₆alkyl, a cycloalkyl, an aryl, a heteroaryl, and a fused ring moiety.

In a 63rd aspect of the second embodiment W is CH₂, CHR₄, or CHR₅CHR₆, Z is ˜T-A-Xₙ—B, and T is —(CH₂)ₚ—(C(O))_{q}— (p is 0 and q is 1), and the compound of formula II is represented by formula (II-10)

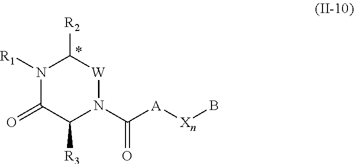

wherein
R₁ is selected from among hydrogen, a C₁₋₆alkyl and a C₁₋₃alkaryl;
R₂, R₃, R₄, R₅, and R₆ are independently selected from among hydrogen, a C₁₋₆alkyl, allyl, a C₃₋₆cycloalkyl, a C₁₋₆alkylene-C₃₋₆cycloalkyl, a C₁₋₆alkylenethioC₁₋₆alkyl, a C₁₋₆alkyleneoxoC₁₋₆alkyl, a C₁₋₆alkylene-C₃₋₆cycloalkyl, an aryl, a C₁₋₃alkaryl, a heteroaryl, and a C₁₋₃alkheteroaryl;
X is CH₂, O, S, or NH, where n is 0 or 1;
A is selected from among —CR′═CR″—, —C≡C—, a C₃₋₆cycloalkylene, an arylene, and a heteroarylene,
   where R′ and R″ are independently selected from among hydrogen, a C₁₋₆alkyl, and an aryl; and
B is selected from among hydrogen, a C₁₋₆alkyl, a C₃₋₆cycloalkyl, an aryl, a heteroaryl, and a fused ring moiety.

In a 64th aspect of the second embodiment W is CH₂, CHR₄, or CHR₅CHR₆, Z is ˜T-A-Xₙ—B, and T is —(CH₂)ₚ—(C(O))_{q}— (p is 0 and q is 1), and the compound of formula II is represented by formula (II-10)

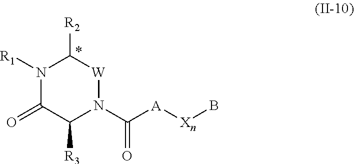

wherein
R₁ is hydrogen or a C₁₋₆alkyl;
R₂, R₃, R₄, R₅, and R₆ are independently selected from among hydrogen, a C₁₋₆alkyl, a C₃₋₆cycloalkyl, a C₁₋₆alkylene-C₃₋₆cycloalkyl, a C₁₋₆alkylenethioC₁₋₆alkyl, a C₁₋₆alkyleneoxoC₁₋₆alkyl, a C₁₋₆alkylene-C₃₋₆cycloalkyl, an aryl, a C₁₋₃alkaryl, a heteroaryl, and a C₁₋₃alkheteroaryl;
X is CH₂, O, S, or NH, where n is 0 or 1;
A is selected from among —CR′═CR″—, —C≡C—, a C₃₋₆cycloalkylene, an arylene, and a heteroarylene,
   where R′ and R″ are independently selected from among hydrogen and a C₁₋₆alkyl; and
B is selected from among hydrogen, a C₁₋₆alkyl, a C₃₋₆cycloalkyl, an aryl, a heteroaryl, and a fused ring moiety.

In a 65th aspect of the second embodiment W is CH₂, CHR₄, or CHR₅CHR₆, Z is ˜T-A-Xₙ—B, and T is —(CH₂)ₚ—(C(O))_{q}— (p is 0 and q is 1), and the compound of formula II is represented by formula (II-10)

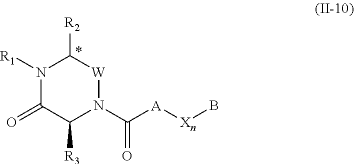

wherein
R₁ is hydrogen or a C₁₋₆alkyl;
R₂, R₃, R₄, R₅, and R₆ are independently selected from among hydrogen, a C₁₋₆alkyl, a C₃₋₆cycloalkyl, a C₁₋₆alkylene-C₃₋₆cycloalkyl, a C₁₋₆alkylenethioC₁₋₆alkyl, a C₁₋₆alkyleneoxoC₁₋₆alkyl, a C₁₋₆alkylene-C₃₋₆cycloalkyl, an aryl, a C₁₋₃alkaryl, a heteroaryl, and a C₁₋₃alkheteroaryl;
X is CH₂, O, S, or NH, where n is 0 or 1;
   A is a heteroarylene; and
B is selected from among hydrogen, a C₁₋₆alkyl, a C₃₋₆cycloalkyl, an aryl, a heteroaryl, and a fused ring moiety.

In a 66th aspect of the second embodiment W is CH₂, CHR₄, or CHR₅CHR₆, Z is ˜T-A-Xₙ—B, and T is —(CH₂)ₚ—(C(O))_{q}— (p is 0 and q is 1), and the compound of formula II is represented by formula (II-10)

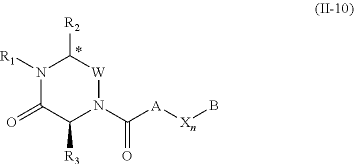

wherein
R₁ is hydrogen or a C₁₋₆alkyl;
R₂, R₃, R₄, R₅, and R₆ are independently selected from among hydrogen, a C₁₋₆alkyl, a C₃₋₆cycloalkyl, a C₁₋₆alkylene-C₃₋₆cycloalkyl, a C₁₋₆alkylenethioC₁₋₆alkyl, a C₁₋₆alkyleneoxoC₁₋₆alkyl, a C₁₋₆alkylene-C₃₋₆cycloalkyl, an aryl, a C₁₋₃alkaryl, a heteroaryl, and a C₁₋₃alkheteroaryl;
where n is 0 and B is bound to A and where;
A is selected from an A′-ring, as defined herein; and
B is selected from among hydrogen, a C₁₋₆alkyl, a C₃₋₆cycloalkyl, an aryl, a heteroaryl, and a fused ring moiety.

In a 67th aspect of the second embodiment W is CH₂, CHR₄, or CHR₅CHR₆, Z is ˜T-A-Xₙ—B, and T is —(CH₂)ₚ—(C(O))_{q}— (p is 0 and q is 1), and the compound of formula II is represented by formula (II-10)

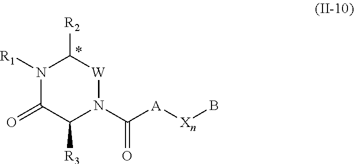

wherein
R₁ is hydrogen or a C₁₋₆alkyl;
R₂, R₃, R₄, R₅, and R₆ are independently selected from among hydrogen, a C₁₋₆alkyl, a C₃₋₆cycloalkyl, a C₁₋₆alkylene-C₃₋₆cycloalkyl, a C₁₋₆alkylenethioC₁₋₆alkyl, a C₁₋₆alkyleneoxoC₁₋₆alkyl, a C₁₋₆alkylene-C₃₋₆cycloalkyl, an aryl, a C₁₋₃alkaryl, a heteroaryl, and a C₁₋₃alkheteroaryl;
where n is 0 and B is bound to A and where;
A is selected from an A′-ring, as defined herein; and
B is selected from a B′-ring, as defined herein.

In a 68th aspect of the second embodiment W is CH₂, CHR₄, or CHR₅CHR₆, Z is ˜T-A-Xₙ—B, and T is —(CH₂)ₚ—(C(O))_{q}— (p is 0 and q is 1), and the compound of formula II is represented by formula (II-10)

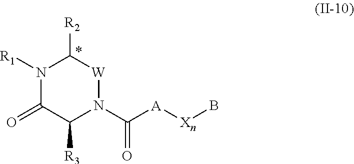

wherein
R₁ is hydrogen;
R₂ is selected from among an R₂-substituent, as defined herein;
R₃ is selected from among CH₃, CH₂CH₃, CH₂CH₂CH₃, CH₂OCH₃, CH₂SCH₃, CH(CH₃)₂, CH(CH₃)(CH₂CH₃), C(CH₃)₃, CH₂CH(CH₃)₂, CH₂C(CH₃)₃, CH═CHCH₃, CH═CH, CH₂cPr, CH₂cBu, CH₂cPn, CH₂cHx, cPr, cBu, cPn, cHx, and CH₂Ph;
each of R₄, R₅, and R₆ is hydrogen;
where n is 0 and B is bound to A and where;
A is selected from an A′-ring, as defined herein; and
B is selected from a B′-ring, as defined herein.

In a sub-aspect of each of the 61st, 62nd, 63rd, 64th, 65th, 66th, 67th, and 68th aspects of the second embodiment, W is CH₂ and R₂ has a configuration as shown in formula (II-10′)

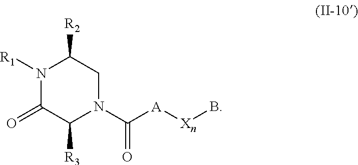

and R₃ is selected from among its respective listing in each of the above-mentioned aspects.

In a 69th aspect of the second embodiment W is CH₂, CHR₄, or CHR₅CHR₆, Z is ˜T-A-Xₙ—B, T is —(CH₂)ₚ—(C(O))_{q}— (with p is 0 and q is 1), and n is 0, and the compound of formula II is represented by formula (II-11)

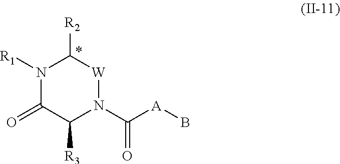

wherein
R₁ is selected from among hydrogen, an alkyl, an alkaryl, an acyl, an aryl, and a heteroaryl;
R₂, R₃, R₄, R₅, and R₆ are independently selected from among hydrogen, an alkyl, allyl, halo, a cycloalkyl, an alkylene-cycloalkyl, an alkylenethioalkyl, an alkyleneoxoalkyl, an alkylene-cycloalkyl, an aryl, an alkaryl, a heteroaryl, an alkheteroaryl, COOH, COOalkyl, CONHalkyl, and CON(alkyl)₂;
A is selected from among —CR′═CR″—, —C≡C—, a cycloalkylene, an arylene, and a heteroarylene,
   where R′ and R″ are independently selected from among hydrogen, a C₁₋₆alkyl, and an aryl; and
B is selected from among hydrogen, a C₁₋₆alkyl, a cycloalkyl, an aryl, a heteroaryl, and a fused ring moiety.

In an 70th aspect of the second embodiment W is CH₂, CHR₄, or CHR₅CHR₆, Z is ˜T-A-Xₙ—B, T is —(CH₂)ₚ—(C(O))_{q}— (with p is 0 and q is 1), and n is 0, and the compound of formula II is represented by formula (II-11)

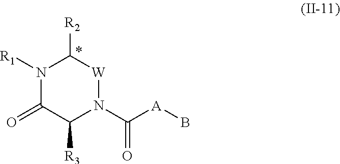

wherein
R₁ is selected from among hydrogen, an alkyl, an alkaryl, an acyl, an aryl, and a heteroaryl;
R₂, R₃, R₄, R₅, and R₆ are independently selected from among hydrogen, an alkyl, allyl, halo, a cycloalkyl, an alkylene-cycloalkyl, an alkylenethioalkyl, an alkyleneoxoalkyl, an alkylene-cycloalkyl, an aryl, an alkaryl, a heteroaryl, and an alkheteroaryl;
A is selected from among —CR′═CR″—, —C≡C—, a cycloalkylene, an arylene, and a heteroarylene,
   where R′ and R″ are independently selected from among hydrogen, a C₁₋₆alkyl, and an aryl; and
B is selected from among hydrogen, a C₁₋₆alkyl, a cycloalkyl, an aryl, a heteroaryl, and a fused ring moiety.

In a 71st aspect of the second embodiment W is CH₂, CHR₄, or CHR₅CHR₆, Z is ˜T-A-Xₙ—B, T is —(CH₂)ₚ—(C(O))_{q}— (with p is 0 and q is 1), and n is 0, and the compound of formula II is represented by formula (II-11)

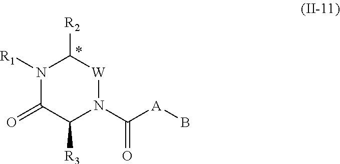

wherein
R₁ is selected from among hydrogen, a C₁₋₆alkyl and a C₁₋₃alkaryl;
R₂, R₃, R₄, R₅, and R₆ are independently selected from among hydrogen, a C₁₋₆alkyl, allyl, a C₃₋₆cycloalkyl, a C₁₋₆alkylene-C₃₋₆cycloalkyl, a C₁₋₆alkylenethioC₁₋₆alkyl, a C₁₋₆alkyleneoxoC₁₋₆alkyl, a C₁₋₆alkylene-C₃₋₆cycloalkyl, an aryl, a C₁₋₃alkaryl, a heteroaryl, and a C₁₋₃alkheteroaryl;
A is selected from among —CR′═CR″—, —C≡C—, a C₃₋₆cycloalkylene, an arylene, and a heteroarylene,
   where R′ and R″ are independently selected from among hydrogen, a C₁₋₆alkyl, and an aryl; and
B is selected from among hydrogen, a C₁₋₆alkyl, a C₃₋₆cycloalkyl, an aryl, a heteroaryl, and a fused ring moiety.

In a 72nd aspect of the second embodiment W is CH₂, CHR₄, or CHR₅CHR₆, Z is ˜T-A-Xₙ—B, T is —(CH₂)ₚ—(C(O))_{q}— (with p is 0 and q is 1), and n is 0, and the compound of formula II is represented by formula (II-11)

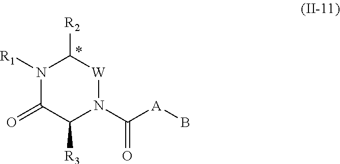

wherein
R₁ is hydrogen or a C₁₋₆alkyl;
R₂, R₃, R₄, R₅, and R₆ are independently selected from among hydrogen, a C₁₋₆alkyl, a C₃₋₆cycloalkyl, a C₁₋₆alkylene-C₃₋₆cycloalkyl, a C₁₋₆alkylenethioC₁₋₆alkyl, a C₁₋₆alkyleneoxoC₁₋₆alkyl, a C₁₋₆alkylene-C₃₋₆cycloalkyl, an aryl, a C₁₋₃alkaryl, a heteroaryl, and a C₁₋₃alkheteroaryl;
A is selected from among —CR′═CR″—, —C≡C—, a C₃₋₆cycloalkylene, an arylene, and a heteroarylene,
   where R′ and R″ are independently selected from among hydrogen and a C₁₋₆alkyl; and
B is selected from among hydrogen, a C₁₋₆alkyl, a C₃₋₆cycloalkyl, an aryl, a heteroaryl, and a fused ring moiety.

In a 73rd aspect of the second embodiment W is CH₂, CHR₄, or CHR₅CHR₆, Z is ˜T-A-Xₙ—B, T is —(CH₂)ₚ—(C(O))_{q}— (with p is 0 and q is 1), and n is 0, and the compound of formula II is represented by formula (II-11)

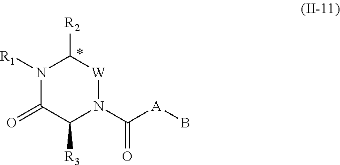

wherein
R₁ is hydrogen or a C₁₋₆alkyl;
R₂, R₃, R₄, R₅, and R₆ are independently selected from among hydrogen, a C₁₋₆alkyl, a C₃₋₆cycloalkyl, a C₁₋₆alkylene-C₃₋₆cycloalkyl, a C₁₋₆alkylenethioC₁₋₆alkyl, a C₁₋₆alkyleneoxoC₁₋₆alkyl, a C₁₋₆alkylene-C₃₋₆cycloalkyl, an aryl, a C₁₋₃alkaryl, a heteroaryl, a C₁₋₃alkheteroaryl;
A is a heteroarylene; and
B is selected from among hydrogen, a C₁₋₆alkyl, a C₃₋₆cycloalkyl, an aryl, a heteroaryl, and a fused ring moiety.

In a 74th aspect of the second embodiment W is CH₂, CHR₄, or CHR₅CHR₆, Z is ˜T-A-Xₙ—B, T is —(CH₂)ₚ—(C(O))_{q}— (with p is 0 and q is 1), and n is 0, and the compound of formula II is represented by formula (II-11)

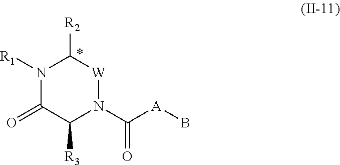

wherein
R₁ is hydrogen or a C₁₋₆alkyl;
R₂, R₃, R₄, R₅, and R₆ are independently selected from among hydrogen, a C₁₋₆alkyl, a C₃₋₆cycloalkyl, a C₁₋₆alkylene-C₃₋₆cycloalkyl, a C₁₋₆alkylenethioC₁₋₆alkyl, a C₁₋₆alkyleneoxoC₁₋₆alkyl, a C₁₋₆alkylene-C₃₋₆cycloalkyl, an aryl, an C₁₋₃alkaryl, a heteroaryl, and a C₁₋₃alkheteroaryl;
A is selected from an N-ring, as defined herein; and
B is selected from among hydrogen, a C₁₋₆alkyl, a C₃₋₆cycloalkyl, an aryl, a heteroaryl, and a fused ring moiety.

In a 75th aspect of the second embodiment W is CH₂, CHR₄, or CHR₅CHR₆, Z is ˜T-A-Xₙ—B, T is —(CH₂)ₚ—(C(O))_{q}— (with p is 0 and q is 1), and n is 0, and the compound of formula II is represented by formula (II-11)

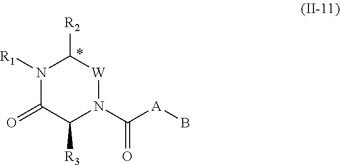

wherein
R₁ is hydrogen or a C₁₋₆alkyl;
R₂, R₃, R₄, R₅, and R₆ are independently selected from among hydrogen, a C₁₋₆alkyl, a C₃₋₆cycloalkyl, a C₁₋₆alkylene-C₃₋₆cycloalkyl, a C₁₋₆alkylenethioC₁₋₆alkyl, a C₁₋₆alkyleneoxoC₁₋₆alkyl, a C₁₋₆alkylene-C₃₋₆cycloalkyl, an aryl, an C₁₋₃alkaryl, a heteroaryl, and a C₁₋₃alkheteroaryl;
A is selected from an A′-ring, as defined herein; and
B is selected from a B′-ring, as defined herein.

In a 76th aspect of the second embodiment W is CH₂, CHR₄, or CHR₅CHR₆, Z is ˜T-A-Xₙ—B, T is —(CH₂)ₚ—(C(O))_{q}— (with p is 0 and q is 1), and n is 0, and the compound of formula II is represented by formula (II-11)

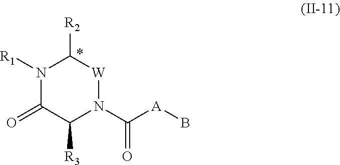

wherein
R₁ is hydrogen;
R₂ is selected from among an R₂-substituent, as defined herein;
R₃ is selected from among CH₃, CH₂CH₃, CH₂CH₂CH₃, CH₂OCH₃, CH₂SCH₃, CH(CH₃)₂, CH(CH₃)(CH₂CH₃), C(CH₃)₃, CH₂CH(CH₃)₂, CH₂C(CH₃)₃, CH═CHCH₃, CH═CH, CH₂cPr, CH₂cBu, CH₂cPn, CH₂cHx, cPr, cBu, cPn, cHx, and CH₂Ph;
each of R₄, R₅, and R₆ is hydrogen;
A is selected from an A′-ring, as defined herein; and
B is selected from a B′-ring, as defined herein.

In a 77th aspect of the second embodiment W is CH₂, Z is ˜T-A-Xₙ—B, T is —(CH₂)ₚ—(C(O))_{q}— (p is 0 and q is 1), and n is 0, and the compound of formula II is represented by formula (II-12)

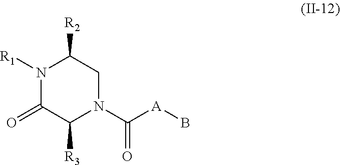

wherein
R₁ is selected from among hydrogen, an alkyl, an alkaryl, an acyl, an aryl, and a heteroaryl;
R₂ and R₃ are independently selected from among, hydrogen, an alkyl, allyl, halo, a cycloalkyl, an alkylene-cycloalkyl, an alkylenethioalkyl, an alkyleneoxoalkyl, an alkylene-cycloalkyl, an aryl, an alkaryl, a heteroaryl, an alkheteroaryl, COOH, COOalkyl, CONHalkyl, and CON(alkyl)₂;
A is selected from among —CR′═CR″—, —C≡C—, a cycloalkylene, an arylene, and a heteroarylene,
   where R′ and R″ are independently selected from among hydrogen, a C₁₋₆alkyl, and an aryl; and
B is selected from among hydrogen, a C₁₋₆alkyl, a cycloalkyl, an aryl, a heteroaryl, and a fused ring moiety.

In an 78th aspect of the second embodiment W is CH₂, Z is ˜T-A-Xₙ—B, T is —(CH₂)ₚ—(C(O))_{q}— (p is 0 and q is 1), and n is 0, and the compound of formula II is represented by formula (II-12)

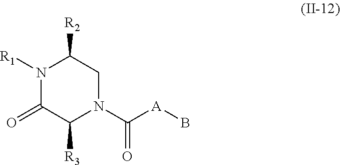

wherein
R₁ is selected from among hydrogen, an alkyl, an alkaryl, an acyl, an aryl, and a heteroaryl;
R₂ and R₃ are independently selected from among, hydrogen, an alkyl, allyl, halo, a cycloalkyl, an alkylene-cycloalkyl, an alkylenethioalkyl, an alkyleneoxoalkyl, an alkylene-cycloalkyl, an aryl, an alkaryl, a heteroaryl, and an alkheteroaryl;
A is selected from among —CR′═CR″—, —C≡C—, a cycloalkylene, an arylene, and a heteroarylene,
   where R′ and R″ are independently selected from among hydrogen, a C₁₋₆alkyl, and an aryl; and
B is selected from among hydrogen, a C₁₋₆alkyl, a cycloalkyl, an aryl, a heteroaryl, and a fused ring moiety.

In a 79th aspect of the second embodiment W is CH₂, Z is ˜T-A-Xₙ—B, T is —(CH₂)ₚ—(C(O))_{q}— (p is 0 and q is 1), and n is 0, and the compound of formula II is represented by formula (II-12)

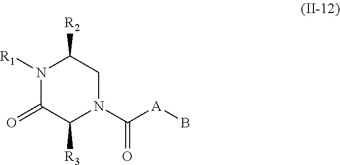

wherein
R₁ is selected from among hydrogen, a C₁₋₆alkyl and a C₁₋₃alkaryl;
R₂ and R₃ are independently selected from among, hydrogen, a C₁₋₆alkyl, allyl, a C₃₋₆cycloalkyl, a C₁₋₆alkylene-C₃₋₆cycloalkyl, a C₁₋₆alkylenethioC₁₋₆alkyl, a C₁₋₆alkyleneoxoC₁₋₆alkyl, a C₁₋₆alkylene-C₃₋₆cycloalkyl, an aryl, an C₁₋₃alkaryl, a heteroaryl, and a C₁₋₃alkheteroaryl;
A is selected from among —CR′═CR″—, —C≡C—, a C₃₋₆cycloalkylene, an arylene, and a heteroarylene,
   where R′ and R″ are independently selected from among hydrogen, a C₁₋₆alkyl, and an aryl; and
B is selected from among hydrogen, a C₁₋₆alkyl, a C₃₋₆cycloalkyl, an aryl, a heteroaryl, and a fused ring moiety.

In a 80th aspect of the second embodiment W is CH₂, Z is ˜T-A-Xₙ—B, T is —(CH₂)ₚ—(C(O))_{q}— (p is 0 and q is 1), and n is 0, and the compound of formula II is represented by formula (II-12)

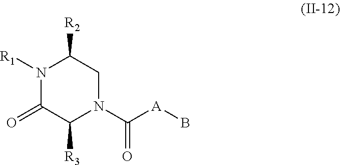

wherein
R₁ is hydrogen or a C₁₋₆alkyl;
R₂ and R₃ are independently selected from among, a C₁₋₆alkyl, a C₃₋₆cycloalkyl, a C₁₋₆alkylene-C₃₋₆cycloalkyl, a C₁₋₆alkylenethioC₁₋₆alkyl, a C₁₋₆alkyleneoxoC₁₋₆alkyl, a C₁₋₆alkylene-C₃₋₆cycloalkyl, an aryl, an C₁₋₃alkaryl, a heteroaryl, and a C₁₋₃alkheteroaryl;
A is selected from among —CR′═CR″—, —C≡C—, a C₃₋₆cycloalkylene, an arylene, and a heteroarylene,
   where R′ and R″ are independently selected from among hydrogen and a C₁₋₆alkyl; and
B is selected from among hydrogen, a C₁₋₆alkyl, a C₃₋₆cycloalkyl, an aryl, a heteroaryl, and a fused ring moiety.

In a 81st aspect of the second embodiment W is CH₂, Z is ˜T-A-Xₙ—B, T is —(CH₂)ₚ—(C(O))_{q}— (p is 0 and q is 1), and n is 0, and the compound of formula II is represented by formula (II-12)

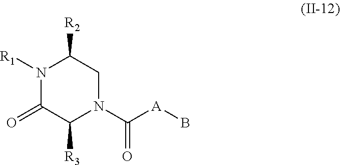

wherein
R₁ is hydrogen or a C₁₋₆alkyl;
R₂ and R₃ are independently selected from among, a C₁₋₆alkyl, a C₃₋₆cycloalkyl, a C₁₋₆alkylene-C₃₋₆cycloalkyl, a C₁₋₆alkylenethioC₁₋₆alkyl, an C₁₋₆alkyleneoxoC₁₋₆alkyl, a C₁₋₆alkylene-C₃₋₆cycloalkyl, an aryl, an C₁₋₃alkaryl, a heteroaryl, and a C₁₋₃alkheteroaryl;
A is a heteroarylene; and
B is selected from among hydrogen, a C₁₋₆alkyl, a C₃₋₆cycloalkyl, an aryl, a heteroaryl, and a fused ring moiety.

In a 82nd aspect of the second embodiment W is CH₂, Z is ˜T-A-Xₙ—B, T is —(CH₂)ₚ—(C(O))_{q}— (p is 0 and q is 1), and n is 0, and the compound of formula II is represented by formula (II-12)

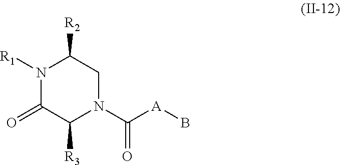

wherein
R₁ is hydrogen or a C₁₋₆alkyl;
R₂ and R₃ are independently selected from among, a C₁₋₆alkyl, a C₃₋₆cycloalkyl, a C₁₋₆alkylene-C₃₋₆cycloalkyl, a C₁₋₆alkylenethioC₁₋₆alkyl, a C₁₋₆alkyleneoxoC₁₋₆alkyl, a C₁₋₆alkylene-C₃₋₆cycloalkyl, an aryl, a C₁₋₃alkaryl, a heteroaryl, and a C₁₋₃alkheteroaryl;
A is selected from an N-ring, as defined herein; and
B is selected from among hydrogen, a C₁₋₆alkyl, a C₃₋₆cycloalkyl, an aryl, a heteroaryl, and a fused ring moiety.

In a 83rd aspect of the second embodiment W is CH₂, Z is ˜T-A-Xₙ—B, T is —(CH₂)ₚ—(C(O))_{q}— (p is 0 and q is 1), and n is 0, and the compound of formula II is represented by formula (II-12)

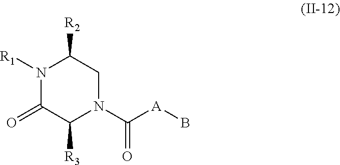

wherein
R₁ is hydrogen or a C₁₋₆alkyl;
R₂ and R₃ are independently selected from among, a C₁₋₆alkyl, a C₃₋₆cycloalkyl, a C₁₋₆alkylene-C₃₋₆cycloalkyl, a C₁₋₆alkylenethioC₁₋₆alkyl, a C₁₋₆alkyleneoxoC₁₋₆alkyl, a C₁₋₆alkylene-C₃₋₆cycloalkyl, an aryl, a C₁₋₃alkaryl, a heteroaryl, and a C₁₋₃alkheteroaryl;
A is selected from an A′-ring, as defined herein; and
B is selected from a B′-ring, as defined herein.

In a 84th aspect of the second embodiment W is CH₂, Z is ˜T-A-Xₙ—B, T is —(CH₂)ₚ—(C(O))_{q}— (p is 0 and q is 1), and n is 0, and the compound of formula II is represented by formula (II-12)

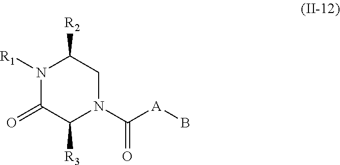

wherein
R₁ is hydrogen;
R₂ and R₃ are independently selected from among, a C₁₋₆alkyl, a C₃₋₆cycloalkyl, a C₁₋₆alkylene-C₃₋₆cycloalkyl, a C₁₋₆alkylenethioC₁₋₆alkyl, a C₁₋₆alkyleneoxoC₁₋₆alkyl, a C₁₋₆alkylene-C₃₋₆cycloalkyl, an aryl, an C₁₋₃alkaryl, a heteroaryl, and a C₁₋₃alkheteroaryl;
A is selected from an A′-ring, as defined herein; and
B is selected from a B′-ring, as defined herein.

In a 85th aspect of the second embodiment W is CH₂, Z is ˜T-A-Xₙ—B, T is —(CH₂)ₚ—(C(O))_{q}— (p is 0 and q is 1), and n is 0, and the compound of formula II is represented by formula (II-12)

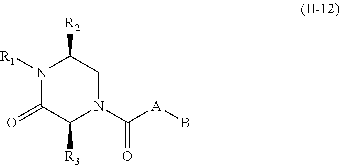

wherein
R₁ is hydrogen;
R₂ is selected from among an R₂-substituent, as defined herein;
R₃ is selected from among CH₃, CH₂CH₃, CH₂CH₂CH₃, CH₂OCH₃, CH₂SCH₃, CH(CH₃)₂, CH(CH₃)(CH₂CH₃), C(CH₃)₃, CH₂CH(CH₃)₂, CH₂C(CH₃)₃, CH═CHCH₃, CH═CH, CH₂cPr, CH₂cBu, CH₂cPn, CH₂cHx, cPr, cBu, cPn, cHx, and CH₂Ph;
A is selected from an A′-ring, as defined herein; and
B is selected from a B′-ring, as defined herein.

An 86th aspect of the second embodiment is directed a compound or its salt thereof selected from among (3S,6R)-4-(5-(4-fluorophenyl)-1,2,4-oxadiazole-3-carbonyl)-3-isobutyl-6-(thiophen-2-yl)piperazin-2-one (430); (3S,6S)-6-(2-chlorothiophen-3-yl)-4-(5-(4-fluorophenyl)isoxazole-3-carbonyl)-3-isobutylpiperazin-2-one (496); (3S,6S)-4-(5-(4-fluorophenyl)-1,2,4-oxadiazole-3-carbonyl)-3-isobutyl-6-(thiophen-3-yl)piperazin-2-one (446); (3S,6R)-4-(5-(4-fluorophenyl)isoxazole-3-carbonyl)-3-isobutyl-6-(thiophen-2-yl)piperazin-2-one (426); (3S,6S)-4-(5-(4-fluorophenyl)isoxazole-3-carbonyl)-3-isobutyl-6-(thiophen-3-yl)piperazin-2-one (436); (3S,6R)-4-(3-(4-fluorophenyl)isoxazole-5-carbonyl)-3-isobutyl-6-(thiophen-2-yl)piperazin-2-one (432); (3S,6S)-6-(2-chlorothiophen-3-yl)-4-(5-(4-fluorophenyl)-1,2,4-oxadiazole-3-carbonyl)-3-isobutylpiperazin-2-one (499); (3S,6R)-4-(3-(4-fluorophenyl)-1,2,4-oxadiazole-5-carbonyl)-3-isobutyl-6-(thiophen-2-yl)piperazin-2-one (431); (3S,6R)-4-(5-(4-fluorophenyl)isoxazole-3-carbonyl)-3-propyl-6-(thiophen-2-yl)piperazin-2-one (494); (3S,6S)-4-(3-(4-Fluorophenyl)-1,2,4-oxadiazole-5-carbonyl)-3-isobutyl-6-phenylpiperazin-2-one (151); (3S,6S)-6-cyclopentyl-4-(3-(4-fluorophenyl)isoxazole-5-carbonyl)-3-isobutylpiperazin-2-one (194); (3S,6S)-4-(3-(4-Fluorophenyl)-1,2,4-oxadiazole-5-carbonyl)-3-isobutyl-6-propylpiperazin-2-one (149); (3S,6S)-4-(5-(4-chlorophenyl)isoxazole-3-carbonyl)-3-isobutyl-6-propylpiperazin-2-one (170); (3S,6S)-4-(5-(4-fluorophenyl)isoxazole-3-carbonyl)-3-isobutyl-6-phenylpiperazin-2-one (178); (3S,6S)-6-cyclohexyl-4-(3-(4-fluorophenyl)isoxazole-5-carbonyl)-3-isobutylpiperazin-2-one (193); (3S,6R)-4-(5-(4-fluorophenyl)-1,2,4-oxadiazole-3-carbonyl)-3-isobutyl-6-(oxazol-5-yl)piperazin-2-one (518); (3S,6S)-4-(3-(4-fluorophenyl)isoxazole-5-carbonyl)-3-isobutyl-6-(thiophen-3-yl)piperazin-2-one (450); (3S,6S)-4-(5-(4-chlorophenyl)isoxazole-3-carbonyl)-3-isobutyl-6-phenylpiperazin-2-one (183); (3S,6S)-6-(2-fluorophenyl)-4-(5-(4-fluorophenyl)-1,2,4-oxadiazole-3-carbonyl)-3-isobutylpiperazin-2-one (370); (3S,6S)-4-(5-(4-fluorophenyl)isoxazole-3-carbonyl)-3-isobutyl-6-((E)-prop-1-en-1-yl)piperazin-2-one (424); (3S,6S)-4-(5-(4-fluorophenyl)isoxazole-3-carbonyl)-3-isobutyl-6-propylpiperazin-2-one (161); (3S,6S)-4-(5-(4-Fluorophenyl)-1,2,4-oxadiazole-3-carbonyl)-3-isobutyl-6-phenylpiperazin-2-one (292); (3S,6S)-4-(5-(4-Fluorophenyl)isoxazol-3-yl)methyl)-3,6-diisobutylpiperazin-2-one (196); (3S,6S)-6-cyclopentyl-4-(5-(4-fluorophenyl)isoxazole-3-carbonyl)-3-isobutyl-piperazin-2-one (163); (3S,6S)-4-((5-(4-fluorophenyl)isoxazol-3-yl)methyl)-3-isobutyl-6-(thiophen-3-yl)piperazin-2-one (442); (3S,6S)-6-(2-fluorophenyl)-4-(5-(4-fluorophenyl)isoxazole-3-carbonyl)-3-isobutyl-piperazin-2-one (355); (3S,6S)-4-(5-(4-Fluorophenyl)isoxazole-3-carbonyl)-3,6-diisobutylpiperazin-2-one (124); (3S,6S)-6-Cyclopentyl-4-(3-(4-fluorophenyl)-1,2,4-oxadiazole-5-carbonyl)-3-isobutylpiperazin-2-one (152); (3S,6S)-4-(3-(4-fluorophenyl)isoxazole-5-carbonyl)-3-isobutyl-6-propylpiperazin-2-one (189); (3S,6S)-4-(3-(4-fluorophenyl)isoxazole-5-carbonyl)-3-isobutyl-6-propylpiperazin-2-one (192); (3S,6S)-4-(5-(4-fluorophenyl)-1,2,4-oxadiazole-3-carbonyl)-3-isobutyl-6-((E)-prop-1-en-1-yl)piperazin-2-one (448); (3S,6R)-4-(5-(4-fluorophenyl)isoxazole-3-carbonyl)-3-isobutyl-6-(oxazol-5-yl)piperazin-2-one (516); (3S,6R)-4-(3-(4-fluorophenyl)isoxazole-5-carbonyl)-3-isobutyl-6-(oxazol-5-yl)piperazin-2-one (519); (3S,6S)-4-(5-(4-chlorophenyl)isoxazole-3-carbonyl)-6-cyclopentyl-3-isobutylpiperazin-2-one (173); (3S,6S)-6-(cyclopropylmethyl)-4-(3-(4-fluorophenyl)isoxazole-5-carbonyl)-3-isobutylpiperazin-2-one (191); (3S,6S)-4-(5-(3,4-difluorophenyl)isoxazole-3-carbonyl)-3-isobutyl-6-propylpiperazin-2-one (184); (3S,6S)-4-(5-(4-fluorophenyl)-1,2,4-oxadiazole-3-carbonyl)-3-isobutyl-6-propylpiperazin-2-one (290); (3S,6S)-4-(3-(4-fluorophenyl)isoxazole-5-carbonyl)-3-isobutyl-6-((E)-prop-1-en-1-yl)piperazin-2-one (447); (3S,6S)-3-isobutyl-6-propyl-4-(5-(thiophen-2-yl)isoxazole-3-carbonyl)piperazin-2-one (169); (3S,6S)-4-(5-(4-chlorophenyl)isoxazole-3-carbonyl)-6-cyclohexyl-3-isobutylpiperazin-2-one (171); (3S,6S)-4-(5-(4-fluorophenyl)isoxazole-3-carbonyl)-3-isobutyl-6-isopropylpiperazin-2-one (175); (3S,6S)-4-(5-(3,4-difluorophenyl)isoxazole-3-carbonyl)-3-isobutyl-6-phenylpiperazin-2-one (188); (3S,6S)-4-(4-fluoro-5-(4-fluorophenyl)isoxazole-3-carbonyl)-6-(2-fluorophenyl)-3-isobutylpiperazin-2-one (380); (3S,6R)-4-(5-(4-fluorophenyl)isoxazole-3-carbonyl)-6-(furan-2-yl)-3-isobutylpiperazin-2-one (484); (3S,6S)-4-((5-(4-fluorophenyl)isoxazol-3-yl)methyl)-3-isobutyl-6-phenylpiperazin-2-one (215); (3S)-4-(5-(4-fluorophenyl)isoxazole-3-carbonyl)-3-isobutyl-6-(tetrahydro-2H-pyran-4-yl)piperazin-2-one (456); (3S,6S)-4-(5-(4-fluorophenyl)isoxazole-3-carbonyl)-3-neopentyl-6-phenylpiperazin-2-one (342); (3S,6S)-4-((5-(4-fluorophenyl)isoxazol-3-yl)methyl)-3-isobutyl-6-propylpiperazin-2-one (206); (3S,6S)-6-cyclopentyl-4-((5-(4-fluorophenyl)isoxazol-3-yl)methyl)-3-isobutylpiperazin-2-one (208); (3S,6S)-6-(2-chlorophenyl)-4-(5-(4-fluorophenyl)isoxazole-3-carbonyl)-3-isobutyl-piperazin-2-one (352); (3S,6S)-4-(4-fluoro-5-(4-fluorophenyl)isoxazole-3-carbonyl)-3,6-diisobutylpiperazin-2-one (379); (3S,6S)-4-(3-(4-Fluorophenyl)-1,2,4-oxadiazole-5-carbonyl)-3-isobutyl-6-isopropylpiperazin-2-one (153); (3S,6S)-4-(3-(4-Fluorophenyl)isoxazole-5-carbonyl)-3-isobutyl-6-phenylpiperazin-2-one (145); (3S,6S)-4-(5-(4-chlorophenyl)isoxazole-3-carbonyl)-3-isobutyl-6-isopropylpiperazin-2-one (181); (3S,6S)-4-(5-(4-chlorophenyl)-1,2,4-oxadiazole-3-carbonyl)-3-isobutyl-6-phenyl-piperazin-2-one (406); (3S,6S)-4-((5-(4-Chlorophenyl)isoxazol-3-yl)methyl)-3,6-diisobutylpiperazin-2-one (197); (3S,6S)-6-cyclopentyl-3-isobutyl-4-(5-(thiophen-2-yl)isoxazole-3-carbonyl)piperazin-2-one (174); (3S,6S)-6-cyclopentyl-4-(4-fluoro-5-(4-fluorophenyl)isoxazole-3-carbonyl)-3-isobutylpiperazin-2-one (381); (3S,6S)-6-cyclobutyl-4-(5-(4-fluorophenyl)-1,2,4-oxadiazole-3-carbonyl)-3-isobutylpiperazin-2-one (467); (3S,6R)-4-(5-(4-fluorophenyl)isoxazole-3-carbonyl)-3-propyl-6-(thiophen-2-yl)piper-azin-2-one (491); (3S,6S)-4-(5-(4-Chlorophenyl)isoxazole-3-carbonyl)-3,6-diisobutylpiperazin-2-one (125); (3S,6S)-6-cyclopentyl-4-(5-(3,4-difluorophenyl)isoxazole-3-carbonyl)-3-isobutylpiperazin-2-one (186); (3S,6S)-4-((5-(4-chlorophenyl)isoxazol-3-yl)methyl)-6-cyclopentyl-3-isobutylpiperazin-2-one (212); (3S,6S)-4-(5-(2,4-difluorophenyl)-4-fluoroisoxazole-3-carbonyl)-3-isobutyl-6-propylpiperazin-2-one (387); (3S,6S)-4-(5-(3,4-difluorophenyl)-4-fluoroisoxazole-3-carbonyl)-3-isobutyl-6-propylpiperazin-2-one (393); (3S,6R)-4-((5-(4-fluorophenyl)isoxazol-3-yl)methyl)-3-isobutyl-6-(thiophen-2-yl)piperazin-2-one (435); (3S,6S)-4-(5-(4-Fluorophenyl)-1,2,4-oxadiazole-3-carbonyl)-3,6-diisobutylpiperazin-2-one (293); (3S,6S)-6-(3-fluorophenyl)-4-(5-(4-fluorophenyl)isoxazole-3-carbonyl)-3-isobutylpiperazin-2-one (367); (3S,6S)-4-(5-(4-fluorophenyl)-1,2,4-oxadiazole-3-carbonyl)-3-isopropyl-6-phenyl-piperazin-2-ono (403); (3S,6R)-4-(5-(2,4-difluorophenyl)isoxazole-3-carbonyl)-3-isobutyl-6-(oxazol-5-yl)piperazin-2-one (522); (3S,6S)-4-(3-(4-chlorophenyl)isoxazole-5-carbonyl)-3-isobutyl-6-phenylpiperazin-2-one (316); (S)-4-(5-(4-fluorophenyl)isoxazole-3-carbonyl)-3,6-diisobutyl-3,4-dihydropyrazin-2(1H)-one (317); (3S,6S)-3-allyl-4-(5-(4-fluorophenyl)-1,2,4-oxadiazole-3-carbonyl)-6-phenyl-piperazin-2-one (465); (3S,6S)-4-[3-(4-Chloro-phenyl)-isoxazole-5-carbonyl]-3,6-diisobutyl-piperazin-2-one (190); (3S,6S)-4-((5-(4-chlorophenyl)isoxazol-3-yl)methyl)-3-isobutyl-6-propylpiperazin-2-one (210); (3S,6S)-4-(5-(4-Fluorophenyl)-1,2,4-oxadiazole-3-carbonyl)-3-isobutyl-6-isopropylpiperazin-2-one (295); (3S,6S)-4-(5-(4-chlorophenyl)-1,2,4-oxadiazole-3-carbonyl)-3,6-diisobutylpiperazin-2-one (405); (3S,6R)-6-(5-chlorothiophen-2-yl)-4-(5-(4-fluorophenyl)isoxazole-3-carbonyl)-3-isobutylpiperazin-2-one (489); (3S,6S)-4-(3-(4-Fluorophenyl)isoxazole-5-carbonyl)-3,6-diisobutylpiperazin-2-one (144); (3S,6S)-6-cyclohexyl-4-(5-(4-fluorophenyl)-1,2,4-oxadiazole-3-carbonyl)-3-isobutylpiperazin-2-one (291); (3S,6S)-4-(5-(2,4-difluorophenyl)isoxazole-3-carbonyl)-3-isobutyl-6-propylpiperazin-2-one (165); (3S,6S)-6-Cyclopentyl-4-(5-(4-fluorophenyl)-1,2,4-oxadiazole-3-carbonyl)-3-isobutylpiperazin-2-one (294); (3S,6S)-4-(5-(4-fluorophenyl)-1,2,4-oxadiazole-3-carbonyl)-3-isobutyl-6-(tetrahydro-2H-pyran-4-yl)piperazin-2-one (474); (3S,6S)-4-(5-(4-chlorophenyl)isoxazole-3-carbonyl)-6-(cyclopropylmethyl)-3-isobutylpiperazin-2-one (166); (3S,6S)-3-(cyclopropylmethyl)-4-(5-(4-fluorophenyl)isoxazole-3-carbonyl)-6-phenylpiperazin-2-one (336); (3S,6S)-4-(3-(4-Fluorophenyl)-1,2,4-oxadiazole-5-carbonyl)-3,6-diisobutylpiperazin-2-one (148); (3S,6S)-6-cyclohexyl-4-(4-fluoro-5-(4-fluorophenyl)isoxazole-3-carbonyl)-3-isobutylpiperazin-2-one (382); (3S,6S)-4-(3-(4-chlorophenyl)isoxazole-5-carbonyl)-3-isobutyl-6-propylpiperazin-2-one (313); (3S,6S)-4-[5-(4-Chloro-3-fluoro-phenyl)-isoxazole-3-carbonyl]-6-cyclopentyl-3-isobutyl-piperazin-2-one (160); (3S,6S)-6-cyclohexyl-3-isobutyl-4-(5-(thiophen-2-yl)isoxazole-3-carbonyl)piperazin-2-one (172); (3S,6S)-4-[5-(4-Chloro-3-fluoro-phenyl)-isoxazole-3-carbonyl]-3-isobutyl-6-propyl-piperazin-2-one (158); (3S,6S)-4-(5-(4-fluorophenyl)isoxazole-3-carbonyl)-6-(cyclopropylmethyl)-3-isobutylpiperazin-2-one (167); (3S,6S)-4-(5-(4-Bromophenyl)isoxazole-3-carbonyl)-3,6-diisobutylpiperazin-2-one (135); (3S,6S)-6-(2-chlorophenyl)-4-(5-(4-fluorophenyl)-1,2,4-oxadiazole-3-carbonyl)-3-isobutylpiperazin-2-one (371); (3S,6S)-6-Cyclopentyl-4-[5-(2,4-difluoro-phenyl)-isoxazole-3-carbonyl]-3-isobutyl-piperazin-2-one (157); (3S,6S)-4-(4-fluoro-5-(4-fluorophenyl)isoxazole-3-carbonyl)-3-isobutyl-6-phenyl-piperazin-2-one (376); (3S,6S)-4-(5-(2,4-difluorophenyl)-4-fluoroisoxazole-3-carbonyl)-3-isobutyl-6-phenylpiperazin-2-one (385); (3S,6S)-4-(3-(4-chlorophenyl)isoxazole-5-carbonyl)-6-cyclopentyl-3-isobutyl-piperazin-2-one (314); (3S,6S)-3,6-Diisobutyl-4-(5-phenylisoxazole-3-carbonyl)piperazin-2-one (108); (3S,6S)-3,6-Diisobutyl-4-(5-(thiophen-2-yl)isoxazole-3-carbonyl)piperazin-2-one (109); (3S,6R)-4-(5-(4-chlorophenyl)isoxazole-3-carbonyl)-3-isobutyl-6-((methylthio)methyl)piperazin-2-one (182); (3S,6S)-4-((5-(4-Bromophenyl)isoxazol-3-yl)methyl)-3,6-diisobutylpiperazin-2-one (198); (3S,6S)-4-(5-(2,4-difluorophenyl)-4-fluoroisoxazole-3-carbonyl)-6-(2-fluorophenyl)-3-isobutylpiperazin-2-one (386); (3R,6S)-4-(5-(4-fluorophenyl)isoxazole-3-carbonyl)-6-phenyl-3-(thiophen-2-yl)piperazin-2-one (471); (3S,6S)-3-Isobutyl-6-phenyl-4-(5-(thiophen-2-yl)isoxazole-3-carbonyl)piperazin-2-one (110); (3S,6S)-3-Isobutyl-4-(3-phenyl-1,2,4-oxadiazole-5-carbonyl)-6-propylpiperazin-2-one (150); (3S,6S)-4-(5-(2,4-difluorophenyl)isoxazole-3-carbonyl)-3,6-diisobutylpiperazin-2-one (133); (3S,6S)-6-cyclopropyl-4-(5-(4-fluorophenyl)isoxazole-3-carbonyl)-3-isobutylpiperazin-2-one (180); (3S,6S)-6-cyclohexyl-4-(5-(3,4-difluorophenyl)isoxazole-3-carbonyl)-3-isobutylpiperazin-2-one (185); (3S,6S)-4-(5-(3,4-difluorophenyl)isoxazole-3-carbonyl)-3-isobutyl-6-isopropylpiperazin-2-one (187); (3S,6S)-6-(4-fluorophenyl)-4-(5-(4-fluorophenyl)isoxazole-3-carbonyl)-3-isobutyl-piperazin-2-one (345); (3S,6S)-6-(2-fluorophenyl)-4-((5-(4-fluorophenyl)isoxazol-3-yl)methyl)-3-isobutyl-piperazin-2-one (375); (3R,6S)-4-(5-(4-fluorophenyl)-1,2,4-oxadiazole-3-carbonyl)-3-((methylthio)methyl)-6-phenylpiperazin-2-one (466); (3S,6S)-4-(5-(3,4-difluorophenyl)-4-fluoroisoxazole-3-carbonyl)-3,6-diisobutyl-piperazin-2-one (394); (3S,6S)-4-((5-(4-chlorophenyl)isoxazol-3-yl)methyl)-6-(cyclopropylmethyl)-3-isobutylpiperazin-2-one (209); (3S,6S)-6-cyclopentyl-4-(5-(2,4-difluorophenyl)-4-fluoroisoxazole-3-carbonyl)-3-isobutylpiperazin-2-ono (388); (3S,6S)-4-(5-(2,4-difluorophenyl)isoxazole-3-carbonyl)-3,6-diisobutylpiperazin-2-one (164); (3S,6R)-4-(5-(4-fluorophenyl)isoxazole-3-carbonyl)-3-isobutyl-6-(5-methylthiophen-2-yl)piperazin-2-one (453); (3S,6S)-6-Cyclopropylmethyl-4-[5-(4-fluoro-phenyl)-isoxazol-3-ylmethyl]-3-isobutyl-piperazin-2-one (203); (3R,6S)-4-(5-(4-fluorophenyl)isoxazole-3-carbonyl)-3-((methylthio)methyl)-6-phenylpiperazin-2-one (468); (3S,6S)-4-(3-(4-chlorophenyl)isoxazole-5-carbonyl)-6-cyclohexyl-3-isobutylpiperazin-2-one (315); (3S,6S)-6-(2-fluorophenyl)-4-(3-(4-fluorophenyl)isoxazole-5-carbonyl)-3-isobutylpiperazin-2-one (372); (3S,6S)-4-((5-(4-fluorophenyl)isoxazol-3-yl)methyl)-3-isobutyl-6-((E)-prop-1-en-1-yl)piperazin-2-one (425); (3S,6R)-6-(5-chlorothiophen-2-yl)-4-(5-(4-fluorophenyl)isoxazole-3-carbonyl)-3-isobutylpiperazin-2-one (488); (3S,6S)-4-[5-(4-Chloro-3-fluoro-phenyl)-isoxazole-3-carbonyl]-6-cyclohexyl-3-isobutyl-piperazin-2-one (159); (3S,6S)-4-(5-(2,4-difluorophenyl)-4-fluoroisoxazole-3-carbonyl)-3,6-diisobutyl-piperazin-2-one (383); (3S,6S)-6-((R)-sec-butyl)-4-(5-(4-fluorophenyl)isoxazol-3-yl)methyl)-3-isobutylpiperazin-2-one (214); (3S,6S)-4-(3-(4-Fluorophenyl)isoxazole-5-carbonyl)-3-isobutyl-6-isopropylpiperazin-2-one (146); (3S,6S)-4-((5-(4-fluorophenyl)isoxazol-3-yl)methyl)-3-isobutyl-6-isopropylpiperazin-2-one (213); (3S,6S)-3-allyl-4-(5-(4-fluorophenyl)isoxazole-3-carbonyl)-6-phenylpiperazin-2-one (407); (3S,6S)-6-cyclobutyl-4-(5-(4-fluorophenyl)isoxazole-3-carbonyl)-3-isobutylpiperazin-2-one (443); (3S,6S)-3,6-Diisobutyl-4-((5-phenylisoxazol-3-yl)methyl)piperazin-2-one (195); (3S,6R)-4-((5-(4-chlorophenyl)isoxazol-3-yl)methyl)-3-isobutyl-6-((methylthio)methyl)piperazin-2-one (218); (3S,6R)-4-(3-(4-Fluorophenyl)-1,2,4-oxadiazole-5-carbonyl)-3-isobutyl-6-((methylthio)methyl)piperazin-2-one (154); (3S,6S)-3-Isobutyl-6-isopropyl-4-(5-(thiophen-2-yl)isoxazole-3-carbonyl)piperazin-2-one (111); 4-(3-((2S,5S)-5-Cyclopentyl-2-isobutyl-3-oxopiperazine-1-carbonyl)isoxazol-5-yl)benzonitrile (137); (3S,6S)-3-((R)-sec-butyl)-4-(5-(4-fluorophenyl)isoxazole-3-carbonyl)-6-phenylpiperazin-2-one (339); (3S,6R)-4-[5-(4-Fluoro-phenyl)-isoxazol-3-ylmethyl]-3-isobutyl-6-methylsulfanylmethyl-piperazin-2-one (217); (3S,6S)-4-(5-(3-Fluorophenyl)isoxazole-3-carbonyl)-3,6-diisobutylpiperazin-2-one (128); (2R,5S)-4-(5-(4-fluorophenyl)isoxazole-3-carbonyl)-5-isobutyl-N,N-dimethyl-6-oxopiperazine-2-carboxamide (507); (3S,6S)-6-cyclohexyl-4-((5-(4-fluorophenyl)isoxazol-3-yl)methyl)-3-isobutylpiperazin-2-one (207); (3S,6S)-3,6-Diisobutyl-4-((5-(thiophen-2-yl)isoxazol-3-yl)methyl)piperazin-2-one (202); (3S,6S)-4-(5-(4-chloro-3-fluorophenyl)isoxazole-3-carbonyl)-3,6-diisobutylpiperazin-2-one (134); (3S,6S)-4-(5-(4-chloro-3-fluorophenyl)isoxazole-3-carbonyl)-3,6-diisobutylpiperazin-2-one (177); (3S,6S)-4-(5-(4-fluorophenyl)isoxazole-3-carbonyl)-6-phenyl-3-propylpiperazin-2-one (397); (3S,6S)-6-(cyclopropylmethyl)-3-isobutyl-4-(5-(thiophen-2-yl)isoxazole-3-carbonyl)piperazin-2-one (168); (3S,6S)-4-(5-(5-Chlorothiophen-2-yl)isoxazole-3-carbonyl)-3,6-diisobutylpiperazin-2-one (112); (3S,6S)-4-(5-(3,4-Difluorophenyl)isoxazole-3-carbonyl)-3,6-diisobutylpiperazin-2-one (123); (3S,6S)-4-(5-(4-fluorophenyl)isoxazole-3-carbonyl)-3-isopropyl-6-phenylpiperazin-2-one (400); (3S,6S)-6-cyclopentyl-4-(5-(3,4-difluorophenyl)-4-fluoroisoxazole-3-carbonyl)-3-isobutylpiperazin-2-one (395); (3S,6S)-4-(5-(4-fluorophenyl)isoxazole-3-carbonyl)-3-isobutyl-6-vinylpipe-razin-2-one (506); (3S,6R)-4-(5-(4-fluorophenyl)isoxazole-3-carbonyl)-3-isobutyl-6-(1H-pyrrol-2-yl)piperazin-2-one (523); (3S,6S)-3,6-Diisobutyl-4-(5-(4-methoxyphenyl)isoxazole-3-carbonyl)piperazin-2-one (127); (3S,6S)-6-Cyclohexyl-4-[5-(2,4-difluoro-phenyl)-isoxazole-3-carbonyl]-3-isobutyl-piperazin-2-one (156); (3R,6S)-4-(5-(4-fluorophenyl)-1,2,4-oxadiazole-3-carbonyl)-6-phenyl-3-(thiophen-2-yl)piperazin-2-one (472); (3S,6S)-4-(5-(4-Chloro-2-fluorophenyl)isoxazole-3-carbonyl)-3,6-diisobutylpiperazin-2-one (143); (3S,6R)-4-(3-(4-Fluorophenyl)isoxazole-5-carbonyl)-3-isobutyl-6-((methylthio)methyl)piperazin-2-one (147); (3S,6S)-6-(2,2-Dimethyl-propyl)-4-[5-(4-fluoro-phenyl)-isoxazol-3-ylmethyl]-3-isobutyl-piperazin-2-one (204); (3S,6S)-4-(5-(4-fluorophenyl)isoxazole-3-carbonyl)-3-isobutyl-6-(o-tolyl)piperazin-2-one (361); (3S,6S)-6-cyclohexyl-4-(5-(2,4-difluorophenyl)-4-fluoroisoxazole-3-carbonyl)-3-isobutylpiperazin-2-one (389); (3S,6S)-4-(3-(4-Chlorophenyl)isothiazole-5-carbonyl)-3,6-diisobutylpiperazin-2-one (105); 4-(3-((2S,5S)-2,5-Diisobutyl-3-oxopiperazine-1-carbonyl)isoxazol-5-yl)benzonitrile (136); (3S,6S)-6-(4-chlorophenyl)-4-(5-(4-fluorophenyl)isoxazole-3-carbonyl)-3-isobutyl-piperazin-2-one(358); (3S,6R)-4-(5-(4-Fluorophenyl)-1,2,4-oxadiazole-3-carbonyl)-3-isobutyl-6-((methylthio)methyl)piperazin-2-one (296); (3S,6S)-4-(3-(4-fluorophenyl)-1,2,4-oxadiazole-5-carbonyl)-3-isopropyl-6-phenyl-piperazin-2-one (404); (3S,6S)-6-cyclopropyl-4-((5-(4-fluorophenyl)isoxazol-3-yl)methyl)-3-isobutylpiperazin-2-one (216); (3S,6S)-6-(cyclopropylmethyl)-4-(5-(2,4-difluorophenyl)isoxazole-3-carbonyl)-3-isobutylpiperazin-2-one (288); (3S,6S)-4-(1-(4-fluorophenyl)-1H-1,2,3-triazole-4-carbonyl)-3,6-diisobutylpiperazin-2-one (351); (3S,6S)-3,6-Diisobutyl-4-(5-(p-tolyl)isoxazole-3-carbonyl)piperazin-2-one (126); (3S,6S)-4-(5-(3-Chloro-4-fluorophenyl)isoxazole-3-carbonyl)-3,6-diisobutylpiperazin-2-one (141); (3S,6S)-6-cyclohexyl-4-(5-(4-fluorophenyl)isoxazole-3-carbonyl)-3-isobutylpiperazin-2-one (162); (3S,6S)-4-(5-(4-fluorophenyl)isoxazole-3-carbonyl)-3-isobutyl-6-(p-tolyl)piperazin-2-one (348); (3S,6S)-6-cyclohexyl-4-(5-(3,4-difluorophenyl)-4-fluoroisoxazole-3-carbonyl)-3-isobutylpiperazin-2-one (396); (3S,6S)-3-allyl-4-(5-(4-fluorophenyl)isoxazole-3-carbonyl)-6-propylpiperazin-2-one (409); (3S,6S)-4-(5-(3,4-difluorophenyl)-4-fluoroisoxazole-3-carbonyl)-6-(2-fluorophenyl)-3-isobutylpiperazin-2-one (392); (3S,6S)-4-(1-(4-chlorophenyl)-1H-1,2,3-triazole-4-carbonyl)-3,6-diisobutylpiperazin-2-one (350); (3S,6S)-4-(5-(2-Fluorophenyl)isoxazole-3-carbonyl)-3,6-diisobutylpiperazin-2-one (142); (3S,6S)-4-(5-(3,4-difluorophenyl)-4-fluoroisoxazole-3-carbonyl)-3-isobutyl-6-phenylpiperazin-2-one (390); (3R,6S)-4-(5-(4-fluorophenyl)isoxazole-3-carbonyl)-3-((methylthio)ethyl)-6-phenylpiperazin-2-one (458); (3S,6S)-3,6-Diisobutyl-4-((5-(p-tolyl)isoxazol-3-yl)methyl)piperazin-2-one (199); (3S,6S)-3,6-Diisobutyl-4-[5-(4-nitro-phenyl)-isoxazole-3-carbonyl]-piperazin-2-one (155); (3S,6S)-4-(5-(3-Chlorophenyl)isoxazole-3-carbonyl)-3,6-diisobutylpiperazin-2-one (129); (3S,6S)-4-(5-(3,4-Dichlorophenyl)isoxazole-3-carbonyl)-3,6-diisobutylpiperazin-2-one (276); (3S,6S)-3,6-diisobutyl-4-(5-(2-methoxyphenyl)isoxazole-3-carbonyl)piperazin-2-one (131); (3S,6S)-4-(4-(4-chlorophenyl)thiophene-2-carbonyl)-3,6-diisobutylpiperazin-2-one (272); (3S,6S)-4-(5-(3,5-difluorophenyl)isoxazole-3-carbonyl)-3,6-diisobutylpiperazin-2-one (287); (3S,6S)-3,6-diisobutyl-4-(5-(4-nitrophenyl)isoxazole-3-carbonyl)piperazin-2-one (273); (3S,6S)-4-(5-(4-(tert-butyl)phenyl)isoxazole-3-carbonyl)-3,6-diisobutylpiperazin-2-one (132); (3S,6S)-4-((5-(4-chlorophenyl)isoxazol-3-yl)methyl)-3-isobutyl-6-phenylpiperazin-2-one (219); (3S,6S)-4-(1-(4-Fluorophenyl)-1H-pyrazole-4-carbonyl)-3,6-diisobutylpiperazin-2-one (104); (3S,6S)-3,6-Diisobutyl-4-(5-(3-methoxyphenyl)isoxazole-3-carbonyl)piperazin-2-one (130); (3S,6S)-3,6-Diisobutyl-4-((5-(4-methoxyphenyl)isoxazol-3-yl)methyl)piperazin-2-one (200); (3S,6S)-4-((5-(Furan-2-yl)isoxazol-3-yl)methyl)-3,6-diisobutylpiperazin-2-one (201); (3S,6S)-3,6-diisobutyl-4-(5-(4-(methylthio)phenyl)isoxazole-3-carbonyl)piperazin-2-one (277); (3S,6S)-4-(5-(2-chlorophenyl)isoxazole-3-carbonyl)-3,6-diisobutylpiperazin-2-one (274); (3S,6S)-4-(5-(4-fluorophenyl)isoxazole-3-carbonyl)-3-isobutyl-6-(4-(trifluoromethyl)-phenyl)piperazin-2-one (366); (3S,6S)-4-(5-(2,4-dichlorophenyl)isoxazole-3-carbonyl)-3,6-diisobutylpiperazin-2-one (275); (3S,6S)-4-(5-(4-fluorophenyl)isoxazole-3-carbonyl)-3-isobutyl-1-methyl-6-phenylpiperazin-2-one (321); (3S,6S)-3,6-Diisobutyl-4-(5-(3-(trifluoromethoxy)phenyl)isoxazole-3-carbonyl)piperazin-2-one (138); (3S,6S)-3,6-Diisobutyl-4-(5-phenylfuran-2-carbonyl)piperazin-2-one (97); (3S,6S)-3,6-Diisobutyl-4-(2-phenylthiazole-4-carbonyl)piperazin-2-one (98); (3S,6S)-4-(5-Ethylisoxazole-3-carbonyl)-3,6-diisobutylpiperazin-2-one (107); (3S,6S)-4-(1-(4-Fluorophenyl)-1H-imidazole-4-carbonyl)-3,6-diisobutylpiperazin-2-one (103); (3S,6S)-4-(5-(4-ethylphenyl)isoxazole-3-carbonyl)-3,6-diisobutylpiperazin-2-one (283); (3S,6S)-4-(5-Cyclopropylisoxazole-3-carbonyl)-3,6-diisobutylpiperazin-2-one (106); (3S,6S)-6-(tert-butyl)-4-((5-(4-fluorophenyl)isoxazol-3-yl)methyl)-3-isobutylpiperazin-2-one (205); (3S,6S)-3,6-Diisobutyl-4-(2-phenyloxazole-4-carbonyl)piperazin-2-one (99); (3S,6S)-3,6-Diisobutyl-4-(2-phenyl-1H-imidazole-4-carbonyl)piperazin-2-one (101); (3S,6S)-3,6-Diisobutyl-4-(1-phenyl-1H-imidazole-4-carbonyl)piperazin-2-one (102); 4-(3-((2S,5S)-2,5-Diisobutyl-3-oxopiperazine-1-carbonyl)isoxazol-5-yl)-N,N-dimethylbenzamide (139); (3S,6S)-4-(5-(4-(Dimethylamino)phenyl)isoxazole-3-carbonyl)-3,6-diisobutylpiperazin-2-one (140); (3S,6S)-4-(5-(4-cyclopropylphenyl)isoxazole-3-carbonyl)-3,6-diisobutylpiperazin-2-one (284); (3S,6S)-3,6-diisobutyl-4-(5-phenyl-1,3,4-oxadiazole-2-carbonyl)piperazin-2-one (285); (3S,6S)-1-ethyl-4-(5-(4-fluorophenyl)isoxazole-3-carbonyl)-3-isobutyl-6-phenylpiperazin-2-one (322); (3S,6S)-4-(5-(4-fluorophenyl)isoxazole-3-carbonyl)-3-isobutyl-6-phenyl-1-propylpiperazin-2-one (323); (3S,7S)-4-(5-(4-fluorophenyl)isoxazole-3-carbonyl)-3,7-diisobutyl-1,4-diazepan-2-one (411); (3S)-4-(5-(4-fluorophenyl)isoxazole-3-carbonyl)-3,6-diisobutyl-1,4-diazepan-2-one (413); (3S,7S)-4-(5-(4-fluorophenyl)isoxazole-3-carbonyl)-3-isobutyl-7-phenyl-1,4-diazepan-2-one (415); (3S,6S)-3-isobutyl-6-phenyl-4-(5-(4-fluorophenyl)isoxazole-3-carbonyl)-1,4-diazepan-2-one (420); (3S,6S)-4-(5-(4-fluorophenyl)isoxazole-3-carbonyl)-3-isobutyl-6-(thiophen-2-yl)piperazin-2-one (428); (3S,6R)-4-(5-(4-fluorophenyl)-1,2,4-oxadiazole-3-carbonyl)-3-isobutyl-6-(3-methylthiophen-2-yl)piperazin-2-one (441); (3S,6R)-4-(5-(4-fluorophenyl)-1,2,4-oxadiazole-3-carbonyl)-3-isobutyl-6-(tetrahydro-2H-pyran-4-yl)piperazin-2-one (473); (3S,6S)-4-(5-(4-fluorophenyl)isoxazole-3-carbonyl)-6-(furan-2-yl)-3-isobutyl-piperazin-2-one (483); (3S,6S)-6-(5-chlorothiophen-2-yl)-4-(5-(4-fluorophenyl)isoxazole-3-carbonyl)-3-isobutylpiperazin-2-one (485); (2R,5S)-4-(5-(4-fluorophenyl)isoxazole-3-carbonyl)-5-isobutyl-N-methyl-6-oxopiperazine-2-carboxamide (508); (3S,6R)-4-(5-(4-fluorophenyl)isoxazole-3-carbonyl)-3-isobutyl-6-(oxazol-2-yl)piperazin-2-one (510); (3S,6S)-4-(5-(4-fluorophenyl)isoxazole-3-carbonyl)-3-isobutyl-6-(1H-pyrrol-2-yl)piperazin-2-one (524); (2R,5S)-1-(5-(4-fluorophenyl)isoxazole-3-carbonyl)-5-isobutyl-2-phenylimidazolidin-4-one (528); (2S,5S)-1-(5-(4-fluorophenyl)isoxazole-3-carbonyl)-5-isobutyl-2-phenylimidazolidin-4-one (529); (3S,6S)-3,6-diisobutyl-4-(5-(4-(trifluoromethoxy)phenyl)isoxazole-3-carbonyl)piperazin-2-one (179); (3S,6S)-3,6-diisobutyl-4-(5-(4-(methylsulfonyl)phenyl)isoxazole-3-carbonyl)piperazin-2-one (176); (3S,6S)-3,6-Diisobutyl-4-(5-phenyl-1H-pyrazole-3-carbonyl)piperazin-2-one (100); (2R,5S)-4-(5-(4-fluorophenyl)isoxazole-3-carbonyl)-5-isobutyl-6-oxopiperazine-2-carboxylic acid (501); and (2R,5S)—N-(2,2-dimethoxyethyl)-4-(5-(4-fluorophenyl)isoxazole-3-carbonyl)-5-isobutyl-6-oxopiperazine-2-carboxamide (509).

An 87th aspect of the second embodiment is directed a compound or its salt thereof selected from among (3S,6R)-4-(5-(4-fluorophenyl)-1,2,4-oxadiazole-3-carbonyl)-3-isobutyl-6-(thiophen-2-yl)piperazin-2-one (430); (3S,6S)-6-(2-chlorothiophen-3-yl)-4-(5-(4-fluorophenyl)isoxazole-3-carbonyl)-3-isobutylpiperazin-2-one (496); (3S,6S)-4-(5-(4-fluorophenyl)-1,2,4-oxadiazole-3-carbonyl)-3-isobutyl-6-(thiophen-3-yl)piperazin-2-one (446); (3S,6R)-4-(5-(4-fluorophenyl)isoxazole-3-carbonyl)-3-isobutyl-6-(thiophen-2-yl)piperazin-2-one (426); (3S,6S)-4-(5-(4-fluorophenyl)isoxazole-3-carbonyl)-3-isobutyl-6-(thiophen-3-yl)piperazin-2-one (436); (3S,6R)-4-(3-(4-fluorophenyl)isoxazole-5-carbonyl)-3-isobutyl-6-(thiophen-2-yl)piperazin-2-one (432); (3S,6S)-6-(2-chlorothiophen-3-yl)-4-(5-(4-fluorophenyl)-1,2,4-oxadiazole-3-carbonyl)-3-isobutylpiperazin-2-one (499); (3S,6R)-4-(3-(4-fluorophenyl)-1,2,4-oxadiazole-5-carbonyl)-3-isobutyl-6-(thiophen-2-yl)piperazin-2-one (431); (3S,6R)-4-(5-(4-fluorophenyl)isoxazole-3-carbonyl)-3-propyl-6-(thiophen-2-yl)piperazin-2-one (494); (3S,6S)-4-(3-(4-Fluorophenyl)-1,2,4-oxadiazole-5-carbonyl)-3-isobutyl-6-phenylpiperazin-2-one (151); (3S,6S)-6-cyclopentyl-4-(3-(4-fluorophenyl)isoxazole-5-carbonyl)-3-isobutylpiperazin-2-one (194); (3S,6S)-4-(3-(4-Fluorophenyl)-1,2,4-oxadiazole-5-carbonyl)-3-isobutyl-6-propylpiperazin-2-one (149); (3S,6S)-4-(5-(4-chlorophenyl)isoxazole-3-carbonyl)-3-isobutyl-6-propylpiperazin-2-one (170); (3S,6S)-4-(5-(4-fluorophenyl)isoxazole-3-carbonyl)-3-isobutyl-6-phenylpiperazin-2-one (178); (3S,6S)-6-cyclohexyl-4-(3-(4-fluorophenyl)isoxazole-5-carbonyl)-3-isobutylpiperazin-2-one (193); (3S,6R)-4-(5-(4-fluorophenyl)-1,2,4-oxadiazole-3-carbonyl)-3-isobutyl-6-(oxazol-5-yl)piperazin-2-one (518); (3S,6S)-4-(3-(4-fluorophenyl)isoxazole-5-carbonyl)-3-isobutyl-6-(thiophen-3-yl)piperazin-2-one (450); (3S,6S)-4-(5-(4-chlorophenyl)isoxazole-3-carbonyl)-3-isobutyl-6-phenylpiperazin-2-one (183); (3S,6S)-6-(2-fluorophenyl)-4-(5-(4-fluorophenyl)-1,2,4-oxadiazole-3-carbonyl)-3-isobutylpiperazin-2-one (370); (3S,6S)-4-(5-(4-fluorophenyl)isoxazole-3-carbonyl)-3-isobutyl-6-((E)-prop-1-en-1-yl)piperazin-2-one (424); (3S,6S)-4-(5-(4-fluorophenyl)isoxazole-3-carbonyl)-3-isobutyl-6-propylpiperazin-2-one (161); (3S,6S)-4-(5-(4-Fluorophenyl)-1,2,4-oxadiazole-3-carbonyl)-3-isobutyl-6-phenylpiperazin-2-one (292); (3S,6S)-4-((5-(4-Fluorophenyl)isoxazol-3-yl)methyl)-3,6-diisobutylpiperazin-2-one (196); (3S,6S)-6-cyclopentyl-4-(5-(4-fluorophenyl)isoxazole-3-carbonyl)-3-isobutyl-piperazin-2-one (163); (3S,6S)-4-((5-(4-fluorophenyl)isoxazol-3-yl)methyl)-3-isobutyl-6-(thiophen-3-yl)piperazin-2-one (442); (3S,6S)-6-(2-fluorophenyl)-4-(5-(4-fluorophenyl)isoxazole-3-carbonyl)-3-isobutyl-piperazin-2-one (355); (3S,6S)-4-(5-(4-Fluorophenyl)isoxazole-3-carbonyl)-3,6-diisobutylpiperazin-2-one (124); (3S,6S)-6-Cyclopentyl-4-(3-(4-fluorophenyl)-1,2,4-oxadiazole-5-carbonyl)-3-isobutylpiperazin-2-one (152); (3S,6S)-4-(3-(4-fluorophenyl)isoxazole-5-carbonyl)-3-isobutyl-6-propylpiperazin-2-one (189); (3S,6S)-4-(3-(4-fluorophenyl)isoxazole-5-carbonyl)-3-isobutyl-6-propylpiperazin-2-one (192); (3S,6S)-4-(5-(4-fluorophenyl)-1,2,4-oxadiazole-3-carbonyl)-3-isobutyl-6-((E)-prop-1-en-1-yl)piperazin-2-one (448); (3S,6R)-4-(5-(4-fluorophenyl)isoxazole-3-carbonyl)-3-isobutyl-6-(oxazol-5-yl)piperazin-2-one (516); (3S,6R)-4-(3-(4-fluorophenyl)isoxazole-5-carbonyl)-3-isobutyl-6-(oxazol-5-yl)piperazin-2-one (519); (3S,6S)-4-(5-(4-chlorophenyl)isoxazole-3-carbonyl)-6-cyclopentyl-3-isobutylpiperazin-2-one (173); (3S,6S)-6-(cyclopropylmethyl)-4-(3-(4-fluorophenyl)isoxazole-5-carbonyl)-3-isobutylpiperazin-2-one (191); (3S,6S)-4-(5-(3,4-difluorophenyl)isoxazole-3-carbonyl)-3-isobutyl-6-propylpiperazin-2-one (184); (3S,6S)-4-(5-(4-fluorophenyl)-1,2,4-oxadiazole-3-carbonyl)-3-isobutyl-6-propylpiperazin-2-one (290); (3S,6S)-4-(3-(4-fluorophenyl)isoxazole-5-carbonyl)-3-isobutyl-6-((E)-prop-1-en-1-yl)piperazin-2-one (447); (3S,6S)-3-isobutyl-6-propyl-4-(5-(thiophen-2-yl)isoxazole-3-carbonyl)piperazin-2-one (169); (3S,6S)-4-(5-(4-chlorophenyl)isoxazole-3-carbonyl)-6-cyclohexyl-3-isobutylpiperazin-2-one (171); (3S,6S)-4-(5-(4-fluorophenyl)isoxazole-3-carbonyl)-3-isobutyl-6-isopropylpiperazin-2-one (175); (3S,6S)-4-(5-(3,4-difluorophenyl)isoxazole-3-carbonyl)-3-isobutyl-6-phenylpiperazin-2-one (188); (3S,6S)-4-(4-fluoro-5-(4-fluorophenyl)isoxazole-3-carbonyl)-6-(2-fluorophenyl)-3-isobutylpiperazin-2-one (380); (3S,6R)-4-(5-(4-fluorophenyl)isoxazole-3-carbonyl)-6-(furan-2-yl)-3-isobutylpiperazin-2-one (484); (3S,6S)-4-((5-(4-fluorophenyl)isoxazol-3-yl)methyl)-3-isobutyl-6-phenylpiperazin-2-one (215); (3S)-4-(5-(4-fluorophenyl)isoxazole-3-carbonyl)-3-isobutyl-6-(tetrahydro-2H-pyran-4-yl)piperazin-2-one (456); (3S,6S)-4-(5-(4-fluorophenyl)isoxazole-3-carbonyl)-3-neopentyl-6-phenylpiperazin-2-one (342); (3S,6S)-4-((5-(4-fluorophenyl)isoxazol-3-yl)methyl)-3-isobutyl-6-propylpiperazin-2-one (206); (3S,6S)-6-cyclopentyl-4-((5-(4-fluorophenyl)isoxazol-3-yl)methyl)-3-isobutylpiperazin-2-one (208); (3S,6S)-6-(2-chlorophenyl)-4-(5-(4-fluorophenyl)isoxazole-3-carbonyl)-3-isobutyl-piperazin-2-one (352); (3S,6S)-4-(4-fluoro-5-(4-fluorophenyl)isoxazole-3-carbonyl)-3,6-diisobutylpiperazin-2-one (379); (3S,6S)-4-(3-(4-Fluorophenyl)-1,2,4-oxadiazole-5-carbonyl)-3-isobutyl-6-isopropylpiperazin-2-one (153); (3S,6S)-4-(3-(4-Fluorophenyl)isoxazole-5-carbonyl)-3-isobutyl-6-phenylpiperazin-2-one (145); (3S,6S)-4-(5-(4-chlorophenyl)isoxazole-3-carbonyl)-3-isobutyl-6-isopropylpiperazin-2-one (181); (3S,6S)-4-(5-(4-chlorophenyl)-1,2,4-oxadiazole-3-carbonyl)-3-isobutyl-6-phenyl-piperazin-2-one (406); (3S,6S)-4-((5-(4-Chlorophenyl)isoxazol-3-yl)methyl)-3,6-diisobutylpiperazin-2-one (197); (3S,6S)-6-cyclopentyl-3-isobutyl-4-(5-(thiophen-2-yl)isoxazole-3-carbonyl)piperazin-2-one (174); (3S,6S)-6-cyclopentyl-4-(4-fluoro-5-(4-fluorophenyl)isoxazole-3-carbonyl)-3-isobutylpiperazin-2-one (381); (3S,6S)-6-cyclobutyl-4-(5-(4-fluorophenyl)-1,2,4-oxadiazole-3-carbonyl)-3-isobutylpiperazin-2-one (467); (3S,6R)-4-(5-(4-fluorophenyl)isoxazole-3-carbonyl)-3-propyl-6-(thiophen-2-yl)piper-azin-2-one (491); (3S,6S)-4-(5-(4-Chlorophenyl)isoxazole-3-carbonyl)-3,6-diisobutylpiperazin-2-one (125); (3S,6S)-6-cyclopentyl-4-(5-(3,4-difluorophenyl)isoxazole-3-carbonyl)-3-isobutylpiperazin-2-one (186); (3S,6S)-4-((5-(4-chlorophenyl)isoxazol-3-yl)methyl)-6-cyclopentyl-3-isobutylpiperazin-2-one (212); (3S,6S)-4-(5-(2,4-difluorophenyl)-4-fluoroisoxazole-3-carbonyl)-3-isobutyl-6-propylpiperazin-2-one (387); (3S,6S)-4-(5-(3,4-difluorophenyl)-4-fluoroisoxazole-3-carbonyl)-3-isobutyl-6-propylpiperazin-2-one (393); (3S,6R)-4-((5-(4-fluorophenyl)isoxazol-3-yl)methyl)-3-isobutyl-6-(thiophen-2-yl)piperazin-2-one (435); (3S,6S)-4-(5-(4-Fluorophenyl)-1,2,4-oxadiazole-3-carbonyl)-3,6-diisobutylpiperazin-2-one (293); (3S,6S)-6-(3-fluorophenyl)-4-(5-(4-fluorophenyl)isoxazole-3-carbonyl)-3-isobutylpiperazin-2-one (367); (3S,6S)-4-(5-(4-fluorophenyl)-1,2,4-oxadiazole-3-carbonyl)-3-isopropyl-6-phenyl-piperazin-2-ono (403); (3S,6R)-4-(5-(2,4-difluorophenyl)isoxazole-3-carbonyl)-3-isobutyl-6-(oxazol-5-yl)piperazin-2-one (522); (3S,6S)-4-(3-(4-chlorophenyl)isoxazole-5-carbonyl)-3-isobutyl-6-phenylpiperazin-2-one (316); (S)-4-(5-(4-fluorophenyl)isoxazole-3-carbonyl)-3,6-diisobutyl-3,4-dihydropyrazin-2(1H)-one (317); (3S,6S)-3-allyl-4-(5-(4-fluorophenyl)-1,2,4-oxadiazole-3-carbonyl)-6-phenyl-piperazin-2-one (465); (3S,6S)-4-[3-(4-Chloro-phenyl)-isoxazole-5-carbonyl]-3,6-diisobutyl-piperazin-2-one (190); (3S,6S)-4-((5-(4-chlorophenyl)isoxazol-3-yl)methyl)-3-isobutyl-6-propylpiperazin-2-one (210); (3S,6S)-4-(5-(4-Fluorophenyl)-1,2,4-oxadiazole-3-carbonyl)-3-isobutyl-6-isopropylpiperazin-2-one (295); (3S,6S)-4-(5-(4-chlorophenyl)-1,2,4-oxadiazole-3-carbonyl)-3,6-diisobutylpiperazin-2-one (405); (3S,6R)-6-(5-chlorothiophen-2-yl)-4-(5-(4-fluorophenyl)isoxazole-3-carbonyl)-3-isobutylpiperazin-2-one (489); (3S,6S)-4-(3-(4-Fluorophenyl)isoxazole-5-carbonyl)-3,6-diisobutylpiperazin-2-one (144); (3S,6S)-6-cyclohexyl-4-(5-(4-fluorophenyl)-1,2,4-oxadiazole-3-carbonyl)-3-isobutylpiperazin-2-one (291); (3S,6S)-4-(5-(2,4-difluorophenyl)isoxazole-3-carbonyl)-3-isobutyl-6-propylpiperazin-2-one (165); (3S,6S)-6-Cyclopentyl-4-(5-(4-fluorophenyl)-1,2,4-oxadiazole-3-carbonyl)-3-isobutylpiperazin-2-one (294); (3S,6S)-4-(5-(4-fluorophenyl)-1,2,4-oxadiazole-3-carbonyl)-3-isobutyl-6-(tetrahydro-2H-pyran-4-yl)piperazin-2-one (474); (3S,6S)-4-(5-(4-chlorophenyl)isoxazole-3-carbonyl)-6-(cyclopropylmethyl)-3-isobutylpiperazin-2-one (166); (3S,6S)-3-(cyclopropylmethyl)-4-(5-(4-fluorophenyl)isoxazole-3-carbonyl)-6-phenylpiperazin-2-one (336); (3S,6S)-4-(3-(4-Fluorophenyl)-1,2,4-oxadiazole-5-carbonyl)-3,6-diisobutylpiperazin-2-one (148); (3S,6S)-6-cyclohexyl-4-(4-fluoro-5-(4-fluorophenyl)isoxazole-3-carbonyl)-3-isobutylpiperazin-2-one (382); (3S,6S)-4-(3-(4-chlorophenyl)isoxazole-5-carbonyl)-3-isobutyl-6-propylpiperazin-2-one (313); (3S,6S)-4-[5-(4-Chloro-3-fluoro-phenyl)-isoxazole-3-carbonyl]-6-cyclopentyl-3-isobutyl-piperazin-2-one (160); (3S,6S)-6-cyclohexyl-3-isobutyl-4-(5-(thiophen-2-yl)isoxazole-3-carbonyl)piperazin-2-one (172); (3S,6S)-4-[5-(4-Chloro-3-fluoro-phenyl)-isoxazole-3-carbonyl]-3-isobutyl-6-propyl-piperazin-2-one (158); (3S,6S)-4-(5-(4-fluorophenyl)isoxazole-3-carbonyl)-6-(cyclopropylmethyl)-3-isobutylpiperazin-2-one (167); (3S,6S)-4-(5-(4-Bromophenyl)isoxazole-3-carbonyl)-3,6-diisobutylpiperazin-2-one (135); (3S,6S)-6-(2-chlorophenyl)-4-(5-(4-fluorophenyl)-1,2,4-oxadiazole-3-carbonyl)-3-isobutylpiperazin-2-one (371); (3S,6S)-6-Cyclopentyl-4-[5-(2,4-difluoro-phenyl)-isoxazole-3-carbonyl]-3-isobutyl-piperazin-2-one (157); (3S,6S)-4-(4-fluoro-5-(4-fluorophenyl)isoxazole-3-carbonyl)-3-isobutyl-6-phenyl-piperazin-2-one (376); (3S,6S)-4-(5-(2,4-difluorophenyl)-4-fluoroisoxazole-3-carbonyl)-3-isobutyl-6-phenylpiperazin-2-one (385); (3S,6S)-4-(3-(4-chlorophenyl)isoxazole-5-carbonyl)-6-cyclopentyl-3-isobutyl-piperazin-2-one (314); (3S,6S)-3,6-Diisobutyl-4-(5-phenylisoxazole-3-carbonyl)piperazin-2-one (108); (3S,6S)-3,6-Diisobutyl-4-(5-(thiophen-2-yl)isoxazole-3-carbonyl)piperazin-2-one (109); (3S,6R)-4-(5-(4-chlorophenyl)isoxazole-3-carbonyl)-3-isobutyl-6-((methylthio)methyl)piperazin-2-one (182); (3S,6S)-4-((5-(4-Bromophenyl)isoxazol-3-yl)methyl)-3,6-diisobutylpiperazin-2-one (198); (3S,6S)-4-(5-(2,4-difluorophenyl)-4-fluoroisoxazole-3-carbonyl)-6-(2-fluorophenyl)-3-isobutylpiperazin-2-one (386); (3R,6S)-4-(5-(4-fluorophenyl)isoxazole-3-carbonyl)-6-phenyl-3-(thiophen-2-yl)piperazin-2-one (471); (3S,6S)-3-Isobutyl-6-phenyl-4-(5-(thiophen-2-yl)isoxazole-3-carbonyl)piperazin-2-one (110); (3S,6S)-3-Isobutyl-4-(3-phenyl-1,2,4-oxadiazole-5-carbonyl)-6-propylpiperazin-2-one (150); (3S,6S)-4-(5-(2,4-difluorophenyl)isoxazole-3-carbonyl)-3,6-diisobutylpiperazin-2-one (133); (3S,6S)-6-cyclopropyl-4-(5-(4-fluorophenyl)isoxazole-3-carbonyl)-3-isobutylpiperazin-2-one (180); (3S,6S)-6-cyclohexyl-4-(5-(3,4-difluorophenyl)isoxazole-3-carbonyl)-3-isobutylpiperazin-2-one (185); (3S,6S)-4-(5-(3,4-difluorophenyl)isoxazole-3-carbonyl)-3-isobutyl-6-isopropylpiperazin-2-one (187); (3S,6S)-6-(4-fluorophenyl)-4-(5-(4-fluorophenyl)isoxazole-3-carbonyl)-3-isobutyl-piperazin-2-one (345); (3S,6S)-6-(2-fluorophenyl)-4-((5-(4-fluorophenyl)isoxazol-3-yl)methyl)-3-isobutyl-piperazin-2-one (375); (3R,6S)-4-(5-(4-fluorophenyl)-1,2,4-oxadiazole-3-carbonyl)-3-((methylthio)methyl)-6-phenylpiperazin-2-one (466); (3S,6S)-4-(5-(3,4-difluorophenyl)-4-fluoroisoxazole-3-carbonyl)-3,6-diisobutyl-piperazin-2-one (394); (3S,6S)-4-((5-(4-chlorophenyl)isoxazol-3-yl)methyl)-6-(cyclopropylmethyl)-3-isobutylpiperazin-2-one (209); (3S,6S)-6-cyclopentyl-4-(5-(2,4-difluorophenyl)-4-fluoroisoxazole-3-carbonyl)-3-isobutylpiperazin-2-ono (388); (3S,6S)-4-(5-(2,4-difluorophenyl)isoxazole-3-carbonyl)-3,6-diisobutylpiperazin-2-one (164); (3S,6R)-4-(5-(4-fluorophenyl)isoxazole-3-carbonyl)-3-isobutyl-6-(5-methylthiophen-2-yl)piperazin-2-one (453); (3S,6S)-6-Cyclopropylmethyl-4-[5-(4-fluoro-phenyl)-isoxazol-3-ylmethyl]-3-isobutyl-piperazin-2-one (203); (3R,6S)-4-(5-(4-fluorophenyl)isoxazole-3-carbonyl)-3-((methylthio)methyl)-6-phenylpiperazin-2-one (468); (3S,6S)-4-(3-(4-chlorophenyl)isoxazole-5-carbonyl)-6-cyclohexyl-3-isobutylpiperazin-2-one (315); (3S,6S)-6-(2-fluorophenyl)-4-(3-(4-fluorophenyl)isoxazole-5-carbonyl)-3-isobutylpiperazin-2-one (372); (3S,6S)-4-((5-(4-fluorophenyl)isoxazol-3-yl)methyl)-3-isobutyl-6-((E)-prop-1-en-1-yl)piperazin-2-one (425); (3S,6R)-6-(5-chlorothiophen-2-yl)-4-(5-(4-fluorophenyl)isoxazole-3-carbonyl)-3-isobutylpiperazin-2-one (488); (3S,6S)-4-[5-(4-Chloro-3-fluoro-phenyl)-isoxazole-3-carbonyl]-6-cyclohexyl-3-isobutyl-piperazin-2-one (159); (3S,6S)-4-(5-(2,4-difluorophenyl)-4-fluoroisoxazole-3-carbonyl)-3,6-diisobutyl-piperazin-2-one (383); (3S,6S)-6-((R)-sec-butyl)-4-((5-(4-fluorophenyl)isoxazol-3-yl)methyl)-3-isobutylpiperazin-2-one (214); (3S,6S)-4-(3-(4-Fluorophenyl)isoxazole-5-carbonyl)-3-isobutyl-6-isopropylpiperazin-2-one (146); (3S,6S)-4-((5-(4-fluorophenyl)isoxazol-3-yl)methyl)-3-isobutyl-6-isopropylpiperazin-2-one (213); (3S,6S)-3-allyl-4-(5-(4-fluorophenyl)isoxazole-3-carbonyl)-6-phenylpiperazin-2-one (407); (3S,6S)-6-cyclobutyl-4-(5-(4-fluorophenyl)isoxazole-3-carbonyl)-3-isobutylpiperazin-2-one (443); (3S,6S)-3,6-Diisobutyl-4-((5-phenylisoxazol-3-yl)methyl)piperazin-2-one (195); (3S,6R)-4-((5-(4-chlorophenyl)isoxazol-3-yl)methyl)-3-isobutyl-6-((methylthio)methyl)piperazin-2-one (218); (3S,6R)-4-(3-(4-Fluorophenyl)-1,2,4-oxadiazole-5-carbonyl)-3-isobutyl-6-((methylthio)methyl)piperazin-2-one (154); (3S,6S)-3-Isobutyl-6-isopropyl-4-(5-(thiophen-2-yl)isoxazole-3-carbonyl)piperazin-2-one (111); 4-(3-((2S,5S)-5-Cyclopentyl-2-isobutyl-3-oxopiperazine-1-carbonyl)isoxazol-5-yl)benzonitrile (137); (3S,6S)-3-((R)-sec-butyl)-4-(5-(4-fluorophenyl)isoxazole-3-carbonyl)-6-phenylpiperazin-2-one (339); (3S,6R)-4-[5-(4-Fluoro-phenyl)-isoxazol-3-ylmethyl]-3-isobutyl-6-methylsulfanylmethyl-piperazin-2-one (217); (3S,6S)-4-(5-(3-Fluorophenyl)isoxazole-3-carbonyl)-3,6-diisobutylpiperazin-2-one (128); (2R,5S)-4-(5-(4-fluorophenyl)isoxazole-3-carbonyl)-5-isobutyl-N,N-dimethyl-6-oxopiperazine-2-carboxamide (507); (3S,6S)-6-cyclohexyl-4-((5-(4-fluorophenyl)isoxazol-3-yl)methyl)-3-isobutylpiperazin-2-one (207); (3S,6S)-3,6-Diisobutyl-4-((5-(thiophen-2-yl)isoxazol-3-yl)methyl)piperazin-2-one (202); (3S,6S)-4-(5-(4-chloro-3-fluorophenyl)isoxazole-3-carbonyl)-3,6-diisobutylpiperazin-2-one (134); (3S,6S)-4-(5-(4-chloro-3-fluorophenyl)isoxazole-3-carbonyl)-3,6-diisobutylpiperazin-2-one (177); (3S,6S)-4-(5-(4-fluorophenyl)isoxazole-3-carbonyl)-6-phenyl-3-propylpiperazin-2-one (397); (3S,6S)-6-(cyclopropylmethyl)-3-isobutyl-4-(5-(thiophen-2-yl)isoxazole-3-carbonyl)piperazin-2-one (168); (3S,6S)-4-(5-(5-Chlorothiophen-2-yl)isoxazole-3-carbonyl)-3,6-diisobutylpiperazin-2-one (112); (3S,6S)-4-(5-(3,4-Difluorophenyl)isoxazole-3-carbonyl)-3,6-diisobutylpiperazin-2-one (123); and (3S,6S)-4-(5-(4-fluorophenyl)isoxazole-3-carbonyl)-3-isopropyl-6-phenylpiperazin-2-one (400).

In a 88th aspect of the second embodiment, W is CH₂, CHR₄, or CHR₅CHR₆, Z is ˜T-A-Xₙ—B, and T is CHR₇—C(O)—, and the compound of formula II is represented by formula (II-13)

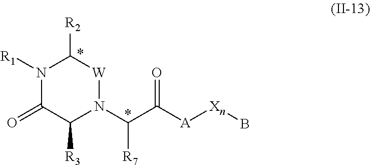

wherein
R₁ is selected from among hydrogen, an alkyl, an alkaryl, an acyl, an aryl, and a heteroaryl;
R₂, R₃, R₄, R₅, and R₆ are independently selected from among hydrogen, an alkyl, allyl, halo, a cycloalkyl, an alkylene-cycloalkyl, an alkylenethioalkyl, an alkyleneoxoalkyl, an alkylene-cycloalkyl, an aryl, an alkaryl, a heteroaryl, an alkheteroaryl, COOH, COOalkyl, CONHalkyl, and CON(alkyl)₂;
R₇ is a C₁₋₆alkyl;
X is CH₂, O, S, or NH, where n is 0 or 1;
A is selected from among —CR′═CR″—, —C≡C—, a cycloalkylene, an arylene, and a heteroarylene,
   where R′ and R″ are independently selected from among hydrogen, a C₁₋₆alkyl, and an aryl; and
B is selected from among hydrogen, a C₁₋₆alkyl, a cycloalkyl, an aryl, a heteroaryl, and a fused ring moiety.

In an 89th aspect of the second embodiment, W is CH₂, CHR₄, or CHR₅CHR₆, Z is ˜T-A-Xₙ—B, and T is CHR₇—C(O)—, and the compound of formula II is represented by formula (II-13)

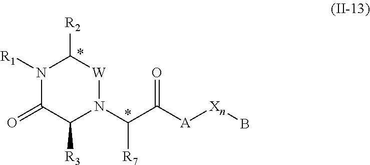

wherein
R₁ is selected from among hydrogen, an alkyl, an alkaryl, an acyl, an aryl, and a heteroaryl;
R₂, R₃, R₄, R₅, and R₆ are independently selected from among hydrogen, an alkyl, allyl, halo, a cycloalkyl, an alkylene-cycloalkyl, an alkylenethioalkyl, an alkyleneoxoalkyl, an alkylene-cycloalkyl, an aryl, an alkaryl, and a heteroaryl, an alkheteroaryl;
R₇ is a C₁₋₆alkyl;
X is CH₂, O, S, or NH, where n is 0 or 1;
A is selected from among —CR′═CR″—, —C≡C—, a cycloalkylene, an arylene, and a heteroarylene,
   where R′ and R″ are independently selected from among hydrogen, a C₁₋₆alkyl, and an aryl; and
B is selected from among hydrogen, a C₁₋₆alkyl, a cycloalkyl, an aryl, a heteroaryl, and a fused ring moiety.

In a 90th aspect of the second embodiment, W is CH₂, CHR₄, or CHR₅CHR₆, Z is ˜T-A-Xₙ—B, and T is CHR₇—C(O)—, and the compound of formula II is represented by formula (II-13)

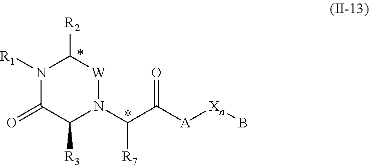

wherein
R₁ is selected from among hydrogen, a C₁₋₆alkyl and a C₁₋₃alkaryl;
R₂, R₃, R₄, R₅, and R₆ are independently selected from among hydrogen, a C₁₋₆alkyl, allyl, a C₃₋₆cycloalkyl, a C₁₋₆alkylene-C₃₋₆cycloalkyl, a C₁₋₆alkylenethioC₁₋₆alkyl, a C₁₋₆alkyleneoxoC₁₋₆alkyl, a C₁₋₆alkylene-C₃₋₆cycloalkyl, an aryl, a C₁₋₃alkaryl, a heteroaryl, and a C₁₋₃alkheteroaryl;
R₇ is a C₁₋₆alkyl;
X is CH₂, O, S, or NH, where n is 0 or 1;
A is selected from among —CR′═CR″—, —C≡C—, a C₃₋₆cycloalkylene, an arylene, and a heteroarylene,
   where R′ and R″ are independently selected from among hydrogen, a C₁₋₆alkyl, and an aryl; and
B is selected from among hydrogen, a C₁₋₆alkyl, a C₃₋₆cycloalkyl, an aryl, a heteroaryl, and a fused ring moiety.

In a 91st aspect of the second embodiment, W is CH₂, CHR₄, or CHR₅CHR₆, Z is ˜T-A-Xₙ—B, and T is CHR₇—C(O)—, and the compound of formula II is represented by formula (II-13)

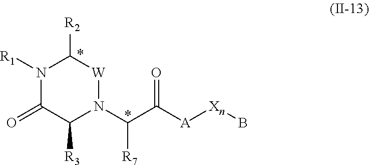

wherein
R₁ is hydrogen or a C₁₋₆alkyl;
R₂, R₃, R₄, R₅, and R₆ are independently selected from among hydrogen, a C₁₋₆alkyl, a C₃₋₆cycloalkyl, a C₁₋₆alkylene-C₃₋₆cycloalkyl, a C₁₋₆alkylenethioC₁₋₆alkyl, a C₁₋₆alkyleneoxoC₁₋₆alkyl, a C₁₋₆alkylene-C₃₋₆cycloalkyl, an aryl, a C₁₋₃alkaryl, a heteroaryl, and a C₁₋₃alkheteroaryl;
R₇ is a C₁₋₆alkyl;
X is CH₂, O, S, or NH, where n is 0 or 1;
A is selected from among —CR′═CR″—, —C≡C—, a C₃₋₆cycloalkylene, an arylene, and a heteroarylene,
   where R′ and R″ are independently selected from among hydrogen and a C₁₋₆alkyl; and
B is selected from among hydrogen, a C₁₋₆alkyl, a C₃₋₆cycloalkyl, an aryl, a heteroaryl, and a fused ring moiety.

In a 92nd aspect of the second embodiment, W is CH₂, CHR₄, or CHR₅CHR₆, Z is ˜T-A-Xₙ—B, and T is CHR₇—C(O)—, and the compound of formula II is represented by formula (II-13)

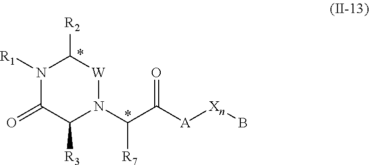

wherein
R₁ is hydrogen or a C₁₋₆alkyl;
R₂, R₃, R₄, R₅, and R₆ are independently selected from among hydrogen, a C₁₋₆alkyl, a C₃₋₆cycloalkyl, a C₁₋₆alkylene-C₃₋₆cycloalkyl, a C₁₋₆alkylenethioC₁₋₆alkyl, a C₁₋₆alkyleneoxoC₁₋₆alkyl, a C₁₋₆alkylene-C₃₋₆cycloalkyl, an aryl, a C₁₋₃alkaryl, a heteroaryl, and a C₁₋₃alkheteroaryl;
R₇ is a C₁₋₆alkyl;
X is CH₂, O, S, or NH, where n is 0 or 1;
   A is a heteroarylene; and
B is selected from among hydrogen, a C₁₋₆alkyl, a C₃₋₆cycloalkyl, an aryl, a heteroaryl, and a fused ring moiety.

In a 93rd aspect of the second embodiment, W is CH₂, CHR₄, or CHR₅CHR₆, Z is ˜T-A-Xₙ—B, and T is CHR₇—C(O)—, and the compound of formula II is represented by formula (II-13)

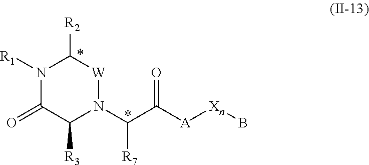

wherein
R₁ is hydrogen or a C₁₋₆alkyl;
R₂, R₃, R₄, R₅, and R₆ are independently selected from among hydrogen, a C₁₋₆alkyl, a C₃₋₆cycloalkyl, a C₁₋₆alkylene-C₃₋₆cycloalkyl, a C₁₋₆alkylenethioC₁₋₆alkyl, a C₁₋₆alkyleneoxoC₁₋₆alkyl, a C₁₋₆alkylene-C₃₋₆cycloalkyl, an aryl, a C₁₋₃alkaryl, a heteroaryl, and a C₁₋₃alkheteroaryl;
R₇ is a C₁₋₆alkyl;
X is CH₂, O, S, or NH, where n is 0 or 1;
A is selected from an A′-ring, as defined herein; and
B is selected from among hydrogen, a C₁₋₆alkyl, a C₃₋₆cycloalkyl, an aryl, a heteroaryl, and a fused ring moiety.

In a 94th aspect of the second embodiment, W is CH₂, CHR₄, or CHR₅CHR₆, Z is ˜T-A-Xₙ—B, and T is CHR₇—C(O)—, and the compound of formula II is represented by formula (II-13)

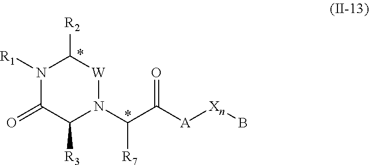

wherein
R₁ is hydrogen or a C₁₋₆alkyl;
R₂, R₃, R₄, R₅, and R₆ are independently selected from among hydrogen, a C₁₋₆alkyl, a C₃₋₆cycloalkyl, a C₁₋₆alkylene-C₃₋₆cycloalkyl, a C₁₋₆alkylenethioC₁₋₆alkyl, a C₁₋₆alkyleneoxoC₁₋₆alkyl, a C₁₋₆alkylene-C₃₋₆cycloalkyl, an aryl, a C₁₋₃alkaryl, a heteroaryl, and a C₁₋₃alkheteroaryl;
R₇ is a C₁₋₆alkyl;
W is CH₂, CHR₄, or CHR₅CHR₆;
X is CH₂, O, S, or NH, where n is 0 or 1;
A is selected from an A′-ring, as defined herein; and
B is selected from a B′-ring, as defined herein.

In a 95th aspect of the second embodiment, W is CH₂, CHR₄, or CHR₅CHR₆, Z is ˜T-A-Xₙ—B, and T is CHR₇—C(O)—, and the compound of formula II is represented by formula (II-13)

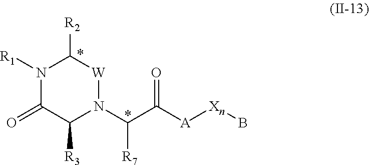

wherein
R₁ is hydrogen;
R₂ is selected from among an R₂-substituent, as defined herein;
R₃ is selected from among CH₃, CH₂CH₃, CH₂CH₂CH₃, CH₂OCH₃, CH₂SCH₃, CH(CH₃)₂, CH(CH₃)(CH₂CH₃), C(CH₃)₃, CH₂CH(CH₃)₂, CH₂C(CH₃)₃, CH═CHCH₃, CH═CH, CH₂cPr, CH₂cBu, CH₂cPn, CH₂cHx, cPr, cBu, cPn, cHx, and CH₂Ph;
each of R₄, R₅, and R₆ is hydrogen;
R₇ is a C₁₋₆alkyl;
X is CH₂, O, S, or NH, where n is 0 or 1;
A is selected from an A′-ring, as defined herein; and
B is selected from a B′-ring, as defined herein.

In a sub-aspect of each of the 88th, 89th, 90th, 91st, 92nd, 93rd, 94th, and 95th aspects of the second embodiment, W is CH₂ and R₂ has a configuration as shown in formula (II-13′)

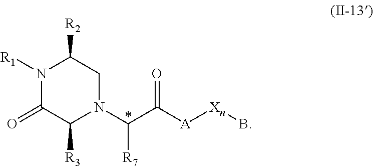

and R₃ is selected from among its respective listing in each of the above-mentioned aspects.

In a 96th aspect of the second embodiment, W is CH₂, CHR₄, or CHR₅CHR₆, Z is ˜T-A-Xₙ—B, T is CHR₇—C(O)— and n is 0, and the compound of formula II is represented by formula (II-14)

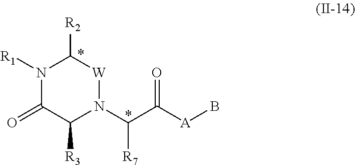

wherein
R₁ is selected from among hydrogen, an alkyl, an alkaryl, an acyl, an aryl, and a heteroaryl;
R₂, R₃, R₄, R₅, and R₆ are independently selected from among hydrogen, an alkyl, allyl, halo, a cycloalkyl, an alkylene-cycloalkyl, an alkylenethioalkyl, an alkyleneoxoalkyl, an alkylene-cycloalkyl, an aryl, an alkaryl, a heteroaryl, an alkheteroaryl, COOH, COOalkyl, CONHalkyl, and CON(alkyl)₂;
R₇ is a C₁₋₆alkyl;
A is selected from among —CR′═CR″—, —C≡C—, a cycloalkylene, an arylene, and a heteroarylene,
   where R′ and R″ are independently selected from among hydrogen, a C₁₋₆alkyl, and an aryl; and
B is selected from among hydrogen, a C₁₋₆alkyl, a cycloalkyl, an aryl, a heteroaryl, and a fused ring moiety.

In an 97th aspect of the second embodiment, W is CH₂, CHR₄, or CHR₅CHR₆, Z is ˜T-A-Xₙ—B, T is CHR₇—C(O)— and n is 0, and the compound of formula II is represented by formula (II-14)

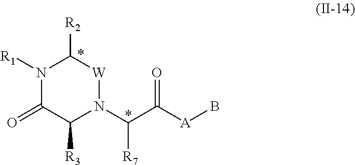

wherein
R₁ is selected from among hydrogen, an alkyl, an alkaryl, an acyl, an aryl, and a heteroaryl;
R₂, R₃, R₄, R₅, and R₆ are independently selected from among hydrogen, an alkyl, allyl, halo, a cycloalkyl, an alkylene-cycloalkyl, an alkylenethioalkyl, an alkyleneoxoalkyl, an alkylene-cycloalkyl, an aryl, an alkaryl, a heteroaryl, and an alkheteroaryl;
R₇ is a C₁₋₆alkyl;
A is selected from among —CR′═CR″—, —C≡C—, a cycloalkylene, an arylene, and a heteroarylene,
   where R′ and R″ are independently selected from among hydrogen, a C₁₋₆alkyl, and an aryl; and
B is selected from among hydrogen, a C₁₋₆alkyl, a cycloalkyl, an aryl, a heteroaryl, and a fused ring moiety.

In a 98th aspect of the second embodiment, W is CH₂, CHR₄, or CHR₅CHR₆, Z is ˜T-A-Xₙ—B, T is CHR₇—C(O)— and n is 0, and the compound of formula II is represented by formula (II-14)

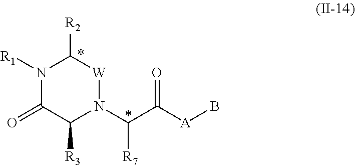

wherein
R₁ is selected from among hydrogen, a C₁₋₆alkyl and a C₁₋₃alkaryl;
R₂, R₃, R₄, R₅, and R₆ are independently selected from among hydrogen, a C₁₋₆alkyl, allyl, a C₃₋₆cycloalkyl, a C₁₋₆alkylene-C₃₋₆cycloalkyl, a C₁₋₆alkylenethioC₁₋₆alkyl, a C₁₋₆alkyleneoxoC₁₋₆alkyl, a C₁₋₆alkylene-C₃₋₆cycloalkyl, an aryl, a C₁₋₃alkaryl, a heteroaryl, and a C₁₋₃alkheteroaryl;
R₇ is a C₁₋₆alkyl;
A is selected from among —CR′═CR″—, —C≡C—, a C₃₋₆cycloalkylene, an arylene, and a heteroarylene,
   where R′ and R″ are independently selected from among hydrogen, a C₁₋₆alkyl, and an aryl; and
B is selected from among hydrogen, a C₁₋₆alkyl, a C₃₋₆cycloalkyl, an aryl, a heteroaryl, and a fused ring moiety.

In a 99th aspect of the second embodiment, W is CH₂, CHR₄, or CHR₅CHR₆, Z is ˜T-A-Xₙ—B, T is CHR₇—C(O)— and n is 0, and the compound of formula II is represented by formula (II-14)

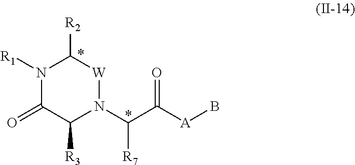

wherein
R₁ is hydrogen or a C₁₋₆alkyl;
R₂, R₃, R₄, R₅, and R₆ are independently selected from among hydrogen, a C₁₋₆alkyl, a C₃₋₆cycloalkyl, a C₁₋₆alkylene-C₃₋₆cycloalkyl, a C₁₋₆alkylenethioC₁₋₆alkyl, a C₁₋₆alkyleneoxoC₁₋₆alkyl, a C₁₋₆alkylene-C₃₋₆cycloalkyl, an aryl, an C₁₋₃alkaryl, a heteroaryl, and a C₁₋₃alkheteroaryl;
R₇ is a C₁₋₆alkyl;
A is selected from among —CR′═CR″—, —C≡C—, a C₃₋₆cycloalkylene, an arylene, and a heteroarylene,
   where R′ and R″ are independently selected from among hydrogen and a C₁₋₆alkyl; and
B is selected from among hydrogen, a C₁₋₆alkyl, a C₃₋₆cycloalkyl, an aryl, a heteroaryl, and a fused ring moiety.

In a 100th aspect of the second embodiment, W is CH₂, CHR₄, or CHR₅CHR₆, Z is ˜T-A-Xₙ—B, T is CHR₇—C(O)— and n is 0, and the compound of formula II is represented by formula (II-14)

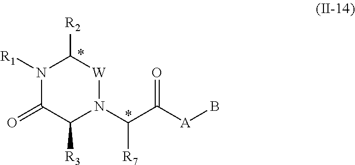

wherein
R₁ is hydrogen or a C₁₋₆alkyl;
R₂, R₃, R₄, R₅, and R₆ are independently selected from among hydrogen, a C₁₋₆alkyl, a C₃₋₆cycloalkyl, a C₁₋₆alkylene-C₃₋₆cycloalkyl, a C₁₋₆alkylenethioC₁₋₆alkyl, a C₁₋₆alkyleneoxoC₁₋₆alkyl, a C₁₋₆alkylene-C₃₋₆cycloalkyl, an aryl, a C₁₋₃alkaryl, a heteroaryl, and a C₁₋₃alkheteroaryl;
R₇ is a C₁₋₆alkyl;
A is a heteroarylene; and
B is selected from among hydrogen, a C₁₋₆alkyl, a C₃₋₆cycloalkyl, an aryl, a heteroaryl, and a fused ring moiety.

In a 101st aspect of the second embodiment, W is CH₂, CHR₄, or CHR₅CHR₆, Z is ˜T-A-Xₙ—B, T is CHR₇—C(O)— and n is 0, and the compound of formula II is represented by formula (II-14)

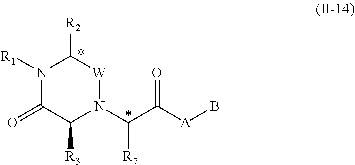

wherein
R₁ is hydrogen or a C₁₋₆alkyl;
R₂, R₃, R₄, R₅, and R₆ are independently selected from among hydrogen, a C₁₋₆alkyl, a C₃₋₆cycloalkyl, a C₁₋₆alkylene-C₃₋₆cycloalkyl, a C₁₋₆alkylenethioC₁₋₆alkyl, a C₁₋₆alkyleneoxoC₁₋₆alkyl, a C₁₋₆alkylene-C₃₋₆cycloalkyl, an aryl, a C₁₋₃alkaryl, a heteroaryl, and a C₁₋₃alkheteroaryl;
R₇ is a C₁₋₆alkyl;
A is selected from an A′-ring, as defined herein; and
B is selected from among hydrogen, a C₁₋₆alkyl, a C₃₋₆cycloalkyl, an aryl, a heteroaryl, and a fused ring moiety.

In a 102nd aspect of the second embodiment, W is CH₂, CHR₄, or CHR₅CHR₆, Z is ˜T-A-Xₙ—B, T is CHR₇—C(O)— and n is 0, and the compound of formula II is represented by formula (II-14)

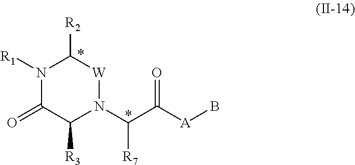

wherein
R₁ is hydrogen or a C₁₋₆alkyl;
R₂, R₃, R₄, R₅, and R₆ are independently selected from among hydrogen, a C₁₋₆alkyl, a C₃₋₆cycloalkyl, a C₁₋₆alkylene-C₃₋₆cycloalkyl, a C₁₋₆alkylenethioC₁₋₆alkyl, a C₁₋₆alkyleneoxoC₁₋₆alkyl, a C₁₋₆alkylene-C₃₋₆cycloalkyl, an aryl, a C₁₋₃alkaryl, a heteroaryl, and a C₁₋₃alkheteroaryl;
R₇ is a C₁₋₆alkyl;
A is selected from an A′-ring, as defined herein; and
B is selected from a B′-ring, as defined herein.

In a 103rd aspect of the second embodiment, W is CH₂, CHR₄, or CHR₅CHR₆, Z is ˜T-A-Xₙ—B, T is CHR₇—C(O)— and n is 0, and the compound of formula II is represented by formula (II-14)

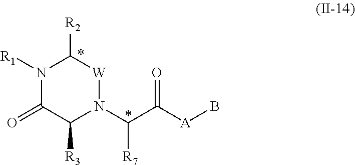

wherein
R₁ is hydrogen;
R₂ is selected from among an R₂-substituent, as defined herein;
R₃ is selected from among CH₃, CH₂CH₃, CH₂CH₂CH₃, CH₂OCH₃, CH₂SCH₃, CH(CH₃)₂, CH(CH₃)(CH₂CH₃), C(CH₃)₃, CH₂CH(CH₃)₂, CH₂C(CH₃)₃, CH═CHCH₃, CH═CH, CH₂cPr, CH₂cBu, CH₂cPn, CH₂cHx, cPr, cBu, cPn, cHx, and CH₂Ph;
each of R₄, R₅, and R₆ is hydrogen;
R₇ is a C₁₋₆alkyl;
A is selected from an A′-ring, as defined herein; and
B is selected from a B′-ring, as defined herein.

In a sub-aspect of each of the 95th, 96th, 97th, 98th, 99th, 100th, 101st, 102nd, and 103rd aspects of the second embodiment, W is CH₂ and R₂ has a configuration as shown in formula (II-14′)

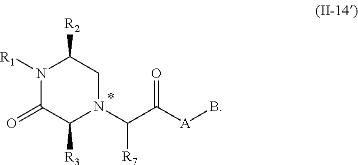

and R₃ is selected from among its respective listing in each of the above-mentioned aspects.

In a third embodiment of compound (A) or its stereoisomer or its salt thereof, W is CH₂, CHR₄, or CHR₅CHR₆, C [symbol] W is a single-bond, Z is ˜T-A-Xₙ—B, n is 0, T is —(CH₂)ₚ—(C(O))_{q}—, p is 0, q is 1, and A is an ethylene, which provides a compound represented by formula III:

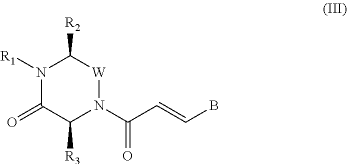

wherein
R₁ is selected from among hydrogen, an alkyl, an alkaryl, an acyl, an aryl, and a heteroaryl;
R₂, R₃, R₄, R₅, and R₆ are independently selected from among hydrogen, an alkyl, allyl, halo, a cycloalkyl, an alkylene-cycloalkyl, an alkylenethioalkyl, an alkyleneoxoalkyl, an alkylene-cycloalkyl, an aryl, an alkaryl, a heteroaryl, an alkheteroaryl, COOH, COOalkyl, CONHalkyl, and CON(alkyl)₂; and
B is selected from among hydrogen, a C₁₋₆alkyl, a cycloalkyl, an aryl, a heteroaryl, and a fused ring moiety.

In a first aspect of the third embodiment,
R₁ is selected from among hydrogen, an alkyl, an alkaryl, an acyl, an aryl, and a heteroaryl;
R₂, R₃, R₄, R₅, and R₆ are independently selected from among hydrogen, an alkyl, allyl, halo, a cycloalkyl, an alkylene-cycloalkyl, an alkylenethioalkyl, an alkyleneoxoalkyl, an alkylene-cycloalkyl, an aryl, an alkaryl, and a heteroaryl, an alkheteroaryl; and
B is selected from among hydrogen, a C₁₋₆alkyl, a cycloalkyl, an aryl, a heteroaryl, and a fused ring moiety.

In a sub-aspect of the first aspect of the third embodiment W is CH₂.

In a second aspect of the third embodiment,
R₁ is selected from among hydrogen, a C₁₋₆alkyl and a C₁₋₃alkaryl;
R₂, R₃, R₄, R₅, and R₆ are independently selected from among hydrogen, a C₁₋₆alkyl, allyl, a C₃₋₆cycloalkyl, a C₁₋₆alkylene-C₃₋₆cycloalkyl, a C₁₋₆alkylenethioC₁₋₆alkyl, an C₁₋₆alkyleneoxoC₁₋₆alkyl, a C₁₋₆alkylene-C₃₋₆cycloalkyl, an aryl, an C₁₋₃alkaryl, a heteroaryl, and a C₁₋₃alkheteroaryl; and
B is selected from among hydrogen, a C₁₋₆alkyl, a C₃₋₆cycloalkyl, an aryl, a heteroaryl, and a fused ring moiety.

In a sub-aspect of the second aspect of the third embodiment W is CH₂.

In a third aspect of the third embodiment,
R₁ is hydrogen or a C₁₋₆alkyl;
R₂, R₃, R₄, R₅, and R₆ are independently selected from among hydrogen, a C₁₋₆alkyl, a C₃₋₆cycloalkyl, a C₁₋₆alkylene-C₃₋₆cycloalkyl, a C₁₋₆alkylenethioC₁₋₆alkyl, a C₁₋₆alkyleneoxoC₁₋₆alkyl, a C₁₋₆alkylene-C₃₋₆cycloalkyl, an aryl, a C₁₋₃alkaryl, a heteroaryl, and a C₁₋₃alkheteroaryl; and
B is selected from among hydrogen, a C₁₋₆alkyl, a C₃₋₆cycloalkyl, an aryl, a heteroaryl, and a fused ring moiety.

In a sub-aspect of the third aspect of the third embodiment W is CH₂.

In a fourth aspect of the third embodiment,
R₁ is hydrogen or a C₁₋₆alkyl;
R₂, R₃, R₄, R₅, and R₆ are independently selected from among hydrogen, a C₁₋₆alkyl, a C₃₋₆cycloalkyl, a C₁₋₆alkylene-C₃₋₆cycloalkyl, a C₁₋₆alkylenethioC₁₋₆alkyl, a C₁₋₆alkyleneoxoC₁₋₆alkyl, a C₁₋₆alkylene-C₃₋₆cycloalkyl, an aryl, a C₁₋₃alkaryl, a heteroaryl, and a C₁₋₃alkheteroaryl; and
B is selected from among hydrogen, a C₁₋₆alkyl, a C₃₋₆cycloalkyl, an aryl, a heteroaryl, and a fused ring moiety.

In a sub-aspect of the fourth aspect of the third embodiment W is CH₂.

In a fifth aspect of the third embodiment,
R₁ is hydrogen or a C₁₋₆alkyl;
R₂, R₃, R₄, R₅, and R₆ are independently selected from among hydrogen, a C₁₋₆alkyl, a C₃₋₆cycloalkyl, a C₁₋₆alkylene-C₃₋₆cycloalkyl, a C₁₋₆alkylenethioC₁₋₆alkyl, a C₁₋₆alkyleneoxoC₁₋₆alkyl, a C₁₋₆alkylene-C₃₋₆cycloalkyl, an aryl, a C₁₋₃alkaryl, a heteroaryl, and a C₁₋₃alkheteroaryl; and
B is selected from among hydrogen, a C₁₋₆alkyl, a C₃₋₆cycloalkyl, an aryl, a heteroaryl, and a fused ring moiety.

In a sub-aspect of the fifth aspect of the third embodiment W is CH₂.

In a sixth aspect of the third embodiment,
R₁ is hydrogen or a C₁₋₆alkyl;
R₂, R₃, R₄, R₅, and R₆ are independently selected from among hydrogen, a C₁₋₆alkyl, a C₃₋₆cycloalkyl, a C₁₋₆alkylene-C₃₋₆cycloalkyl, a C₁₋₆alkylenethioC₁₋₆alkyl, a C₁₋₆alkyleneoxoC₁₋₆alkyl, a C₁₋₆alkylene-C₃₋₆cycloalkyl, an aryl, a C₁₋₃alkaryl, a heteroaryl, and a C₁₋₃alkheteroaryl; and
B is selected from a B′-ring, as defined herein.

In a sub-aspect of the sixth aspect of the third embodiment W is CH₂.

In seventh aspect of the third embodiment,
R₁ is hydrogen;
R₂, R₃, R₄, R₅, and R₆ are independently selected from among hydrogen, a C₁₋₆alkyl, a C₃₋₆cycloalkyl, a C₁₋₆alkylene-C₃₋₆cycloalkyl, a C₁₋₆alkylenethioC₁₋₆alkyl, a C₁₋₆alkyleneoxoC₁₋₆alkyl, a C₁₋₆alkylene-C₃₋₆cycloalkyl, an aryl, a C₁₋₃alkaryl, a heteroaryl, and a C₁₋₃alkheteroaryl; and
B is selected from a B′-ring, as defined herein.

In a sub-aspect of the seventh aspect of the third embodiment W is CH₂.

In an eighth aspect of the third embodiment,
R₁ is hydrogen;
R₂ is selected from among an R₂-substituent, as defined herein;
R₃ is selected from among CH₃, CH₂CH₃, CH₂CH₂CH₃, CH₂OCH₃, CH₂SCH₃, CH(CH₃)₂, CH(CH₃)(CH₂CH₃), C(CH₃)₃, CH₂CH(CH₃)₂, CH₂C(CH₃)₃, CH═CHCH₃, CH═CH, CH₂cPr, CH₂cBu, CH₂cPn, CH₂cHx, cPr, cBu, cPn, cHx, and CH₂Ph;
each of R₄, R₅, and R₆ is hydrogen; and
B is selected from a B′-ring, as defined herein.

In a sub-aspect of the eighth aspect of the third embodiment W is CH₂.

A ninth aspect of the third embodiment is directed to a compound or its salt thereof selected from among (3S,6S)-3,6-Diisobutyl-4-[(E)-(3-phenyl-acryloyl)]-piperazin-2-one (71); (3S,6S)-4-[3-(4-Chloro-phenyl)-acryloyl]-3,6-diisobutyl-piperazin-2-one (72); (3S,6S)-3,6-Diisobutyl-4-[3-(4-trifluoromethyl-phenyl)-acryloyl]-piperazin-2-one (73); (3S,6S)-3-Isobutyl-6-methyl-4-(3-pyridin-3-yl-acryloyl)-piperazin-2-one (74); (3S,6S)-3,6-Diisobutyl-4-(3-phenyl-but-2-enoyl)-piperazin-2-one (75); (3S,6S)-3,6-Diisobutyl-4-(2-methyl-3-phenyl-acryloyl)-piperazin-2-one (78); (3S,6S)-3,6-Diisobutyl-4-(3-p-tolyl-acryloyl)-piperazin-2-one (79); (3S,6S)-4-[3-(4-Fluoro-phenyl)-acryloyl]-3,6-diisobutyl-piperazin-2-one (80); (3S,6S)-4-[3-(3,4-Dichloro-phenyl)-acryloyl]-3,6-diisobutyl-piperazin-2-one (81); (3S,6S)-4-[3-(3,4-Difluoro-phenyl)-acryloyl]-3,6-diisobutyl-piperazin-2-one (82); (3S,6S)-4-[3-(3-Fluoro-phenyl)-acryloyl]-3,6-diisobutyl-piperazin-2-one (83); (3S,6S)-3,6-Diisobutyl-4-[3-(3,4,5-trifluoro-phenyl)-acryloyl]-piperazin-2-one (84); (3S,6S)-4-[3-(3-Chloro-4-fluoro-phenyl)-acryloyl]-3,6-diisobutyl-piperazin-2-one (85); (3S,6S)-4-[3-(4-Chloro-2-fluoro-phenyl)-acryloyl]-3,6-diisobutyl-piperazin-2-one (86); (3S,6S)-4-[3-(2,4-Difluoro-phenyl)-acryloyl]-3,6-diisobutyl-piperazin-2-one (87); (3S,6S)-4-((E)-3-(Benzo[d][1,3]dioxol-5-yl)acryloyl)-3,6-diisobutylpiperazin-2-one (90); (3S,6S)-3,6-Diisobutyl-4-((E)-3-(4-nitrophenyl)acryloyl)piperazin-2-one (91); (3S,6S)-3,6-Diisobutyl-4-((E)-3-(4-(methylsulfonyl)phenyl)acryloyl)piperazin-2-one (92); (3S,6S)-4-((E)-3-(2-Fluoro-4-(trifluoromethyl)phenyl)acryloyl)-3,6-diisobutylpiperazin-2-one (93); (3S,6S)-4-((E)-3-([1,1′-Biphenyl]-4-yl)acryloyl)-3,6-diisobutylpiperazin-2-one (94); (3S,6S)-4-((E)-3-(3,5-Difluorophenyl)acryloyl)-3,6-diisobutylpiperazin-2-one (95); (3S,6S)-4-((E)-3-(2,4-difluorophenyl)acryloyl)-3-isobutyl-6-propylpiperazin-2-one (96); (3S,6S)-4-((E)-3-(4-(dimethylamino)phenyl)acryloyl)-3,6-diisobutylpiperazin-2-one (261); (3S,6S)-3,6-diisobutyl-4-((E)-3-(4-methoxyphenyl)acryloyl)piperazin-2-one (263); (3S,6S)-4-[3-(3-chlorophenyl)-acryloyl]-3,6-diisobutyl-piperazin-2-one (264); (3S,6S)-4-[3-(2-chlorophenyl)-acryloyl]-3,6-diisobutyl-piperazin-2-one (265); (3S,6S)-4-[3-(2-chlorophenyl)-acryloyl]-3,6-diisobutyl-piperazin-2-one (266); (3S,6S)-4-[3-(2,4-dichloro-phenyl)-acryloyl]-3,6-diisobutyl-piperazin-2-one (267); (3S,6S)-3,6-diisobutyl-4-((E)-3-(4-methylsulfanyl-phenyl)acryloyl)piperazin-2-one (268); (3S,6S)-3,6-diisobutyl-4-((E)-3-(4-tert-butyl-phenyl)acryloyl)piperazin-2-one (269); Methyl 4-((E)-3-((2S,5S)-2,5-diisobutyl-3-oxopiperazin-1-yl)-3-oxoprop-1-en-1-yl)-benzoate (270); (3S,6S)-4-[3-(2,6-Difluoro-phenyl)-acryloyl]-3,6-diisobutyl-piperazin-2-one (271); (3S,6S)-4-((E)-3-(2,4-difluorophenyl)acryloyl)-6-(2-fluorophenyl)-3-isobutylpipera-zin-2-one (374); (3S,6R)-4-((E)-3-(2,4-difluorophenyl)acryloyl)-3-isobutyl-6-(thiophen-2-yl)pipera-zin-2-one (434); (3S,6S)-4-((E)-3-(2,4-difluorophenyl)acryloyl)-3-isobutyl-6-((E)-prop-1-en-1-yl)piperazin-2-one (449); (3S,6S)-4-((E)-3-(2,4-difluorophenyl)acryloyl)-3-isobutyl-6-(thiophen-3-yl)piperazin-2-one (451); and (3S,6R)-4-((E)-3-(2,4-difluorophenyl)acryloyl)-3-isobutyl-6-(oxazol-5-yl)piperazin-2-one (520)

In a fourth embodiment of compound (A) or its stereoisomer or its salt thereof, W is CH₂, CHR₄, or CHR₅CHR₆, C [symbol] W is a single bond, Z is ˜T-A-Xₙ—B, n is 0, T is —(CH₂)ₚ—(C(O))_{q}—, p is 0 and q is 1, and A is a cyclopropylene, which provides a compound represented by formula IV:

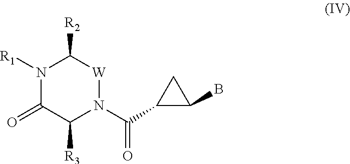

wherein
R₁ is selected from among hydrogen, an alkyl, an alkaryl, an acyl, an aryl, and a heteroaryl;
R₂, R₃, R₄, R₅, and R₆ are independently selected from among hydrogen, an alkyl, allyl, halo, a cycloalkyl, an alkylene-cycloalkyl, an alkylenethioalkyl, an alkyleneoxoalkyl, an alkylene-cycloalkyl, an aryl, an alkaryl, a heteroaryl, an alkheteroaryl, COOH, COOalkyl, CONHalkyl, and CON(alkyl)₂;
B is selected from among hydrogen, a C₁₋₆alkyl, a cycloalkyl, an aryl, a heteroaryl, and a fused ring moiety.

In a first aspect of the fourth embodiment,
R₁ is selected from among hydrogen, an alkyl, an alkaryl, an acyl, an aryl, and a heteroaryl;
R₂, R₃, R₄, R₅, and R₆ are independently selected from among hydrogen, an alkyl, allyl, halo, a cycloalkyl, an alkylene-cycloalkyl, an alkylenethioalkyl, an alkyleneoxoalkyl, an alkylene-cycloalkyl, an aryl, an alkaryl, a heteroaryl, and an alkheteroaryl; and
B is selected from among hydrogen, a C₁₋₆alkyl, a cycloalkyl, an aryl, a heteroaryl, and a fused ring moiety.

In a sub-aspect of the first aspect of the fourth embodiment W is CH₂.

In a second aspect of the fourth embodiment,
R₁ is selected from among hydrogen, a C₁₋₆alkyl and a C₁₋₃alkaryl;
R₂, R₃, R₄, R₅, and R₆ are independently selected from among hydrogen, a C₁₋₆alkyl, allyl, a C₃₋₆cycloalkyl, a C₁₋₆alkylene-C₃₋₆cycloalkyl, a C₁₋₆alkylenethioC₁₋₆alkyl, a C₁₋₆alkyleneoxoC₁₋₆alkyl, a C₁₋₆alkylene-C₃₋₆cycloalkyl, an aryl, a C₁₋₃alkaryl, a heteroaryl, and a C₁₋₃alkheteroaryl; and
B is selected from among hydrogen, a C₁₋₆alkyl, a C₃₋₆cycloalkyl, an aryl, a heteroaryl, and a fused ring moiety.

In a sub-aspect of the second aspect of the fourth embodiment W is CH₂.

In a third aspect of the fourth embodiment,
R₁ is hydrogen or a C₁₋₆alkyl;
R₂, R₃, R₄, R₅, and R₆ are independently selected from among hydrogen, a C₁₋₆alkyl, a C₃₋₆cycloalkyl, a C₁₋₆alkylene-C₃₋₆cycloalkyl, a C₁₋₆alkylenethioC₁₋₆alkyl, a C₁₋₆alkyleneoxoC₁₋₆alkyl, a C₁₋₆alkylene-C₃₋₆cycloalkyl, an aryl, a C₁₋₃alkaryl, a heteroaryl, and a C₁₋₃alkheteroaryl; and
B is selected from among hydrogen, a C₁₋₆alkyl, a C₃₋₆cycloalkyl, an aryl, a heteroaryl, and a fused ring moiety.

In a sub-aspect of the third aspect of the fourth embodiment W is CH₂.

In a fourth aspect of the fourth embodiment,
R₁ is hydrogen or a C₁₋₆alkyl;
R₂, R₃, R₄, R₅, and R₆ are independently selected from among hydrogen, a C₁₋₆alkyl, a C₃₋₆cycloalkyl, a C₁₋₆alkylene-C₃₋₆cycloalkyl, a C₁₋₆alkylenethioC₁₋₆alkyl, a C₁₋₆alkyleneoxoC₁₋₆alkyl, a C₁₋₆alkylene-C₃₋₆cycloalkyl, an aryl, a C₁₋₃alkaryl, a heteroaryl, and a C₁₋₃alkheteroaryl;
B is selected from among hydrogen, a C₁₋₆alkyl, a C₃₋₆cycloalkyl, an aryl, a heteroaryl, and a fused ring moiety.

In a sub-aspect of the fourth aspect of the fourth embodiment W is CH₂.

In a fifth aspect of the fourth embodiment,
R₁ is hydrogen or a C₁₋₆alkyl;
R₂, R₃, R₄, R₅, and R₆ are independently selected from among a C₁₋₆alkyl, a C₃₋₆cycloalkyl, a C₁₋₆alkylene-C₃₋₆cycloalkyl, a C₁₋₆alkylenethioC₁₋₆alkyl, a C₁₋₆alkyleneoxoC₁₋₆alkyl, a C₁₋₆alkylene-C₃₋₆cycloalkyl, an aryl, a C₁₋₃alkaryl, a heteroaryl, and a C₁₋₃alkheteroaryl; and
B is selected from among hydrogen, a C₁₋₆alkyl, a C₃₋₆cycloalkyl, an aryl, a heteroaryl, and a fused ring moiety.

In a sub-aspect of the fifth aspect of the fourth embodiment W is CH₂.

In a sixth aspect of the fourth embodiment,
R₁ is hydrogen or a C₁₋₆alkyl;
R₂, R₃, R₄, R₅, and R₆ are independently selected from among hydrogen, a C₁₋₆alkyl, a C₃₋₆cycloalkyl, a C₁₋₆alkylene-C₃₋₆cycloalkyl, a C₁₋₆alkylenethioC₁₋₆alkyl, a C₁₋₆alkyleneoxoC₁₋₆alkyl, a C₁₋₆alkylene-C₃₋₆cycloalkyl, an aryl, a C₁₋₃alkaryl, a heteroaryl, and a C₁₋₃alkheteroaryl; and
B is selected from a B′-ring, as defined herein.

In a sub-aspect of the sixth aspect of the fourth embodiment W is CH₂.

In seventh aspect of the fourth embodiment,
R₁ is hydrogen;
R₂, R₃, R₄, R₅, and R₆ are independently selected from among hydrogen, a C₁₋₆alkyl, a C₃₋₆cycloalkyl, a C₁₋₆alkylene-C₃₋₆cycloalkyl, a C₁₋₆alkylenethioC₁₋₆alkyl, a C₁₋₆alkyleneoxoC₁₋₆alkyl, a C₁₋₆alkylene-C₃₋₆cycloalkyl, an aryl, an C₁₋₃alkaryl, a heteroaryl, and a C₁₋₃alkheteroaryl; and
B is selected from a B′-ring, as defined herein.

In a sub-aspect of the seventh aspect of the fourth embodiment W is CH₂.

In an eighth aspect of the fourth embodiment,
R₁ is hydrogen;
R₂ is selected from among an R₂-substituent, as defined herein;
R₃ is selected from among CH₃, CH₂CH₃, CH₂CH₂CH₃, CH₂OCH₃, CH₂SCH₃, CH(CH₃)₂, CH(CH₃)(CH₂CH₃), C(CH₃)₃, CH₂CH(CH₃)₂, CH₂C(CH₃)₃, CH═CHCH₃, CH═CH, CH₂cPr, CH₂cBu, CH₂cPn, CH₂cHx, cPr, cBu, cPn, cHx, and CH₂Ph; and
each of R₄, R₅, and R₆ is hydrogen;
B is selected from a B′-ring, as defined herein.

In a sub-aspect of the ninth aspect of the fourth embodiment W is CH₂.

A ninth aspect of the fourth embodiment is directed to a compound or its salt thereof selected from among (3S,6R)-4-((1R,2R)-2-(4-fluorophenyl)cyclopropanecarbonyl)-3-isobutyl-6-(thiophen-2-yl)piperazin-2-one (433); (3S,6S)-6-(2-chlorothiophen-3-yl)-4-((1R,2R)-2-(4-fluorophenyl)cyclopropane-carbonyl)-3-isobutylpiperazin-2-one (500); (3S,6R)-4-((1R,2R)-2-(4-fluorophenyl)cyclopropanecarbonyl)-3-isobutyl-6-(oxazol-5-yl)piperazin-2-one (521); (3S,6S)-4-((1R,2R)-2-(4-fluorophenyl)cyclopropanecarbonyl)-3-isobutyl-6-((E)-prop-1-en-1-yl)piperazin-2-one (452); (3S,6S)-4-[(1R,2R)-2-(4-Fluoro-phenyl)-cyclopropanecarbonyl]-3-isobutyl-6-phenyl-piperazin-2-one (247); (3S,6S)-6-(2-fluorophenyl)-4-((1R,2R)-2-(4-fluorophenyl)cyclopropanecarbonyl)-3-isobutylpiperazin-2-one (373); (3S,6S)-6-Cyclopentyl-4-[(1R,2R)-2-(4-fluoro-phenyl)-cyclopropanecarbonyl]-3-isobutyl-piperazin-2-one (242); (3S,6S)-4-[(1R,2R)-2-(4-Chloro-phenyl)-cyclopropanecarbonyl]-3-isobutyl-6-propyl-piperazin-2-one (244); (3S,6S)-4-[(1R,2R)-2-(4-Fluoro-phenyl)-cyclopropanecarbonyl]-3,6-diisobutyl-piperazin-2-one (236); (3S,6S)-4-[(1R,2R)-2-(4-Fluoro-phenyl)-cyclopropanecarbonyl]-3-isobutyl-6-propyl-piperazin-2-one (240); (3S,6S)-6-Cyclohexyl-4-[(1R,2R)-2-(4-fluoro-phenyl)-cyclopropanecarbonyl]-3-isobutyl-piperazin-2-one (241); (3S,6S)-4-[(1R,2R)-2-(4-Chloro-phenyl)-cyclopropanecarbonyl]-3-isobutyl-6-phenyl-piperazin-2-one (248); (3S,6S)-4-((1R,2R)-2-(4-chlorophenyl)cyclopropanecarbonyl)-3-isobutyl-6-phenyl-piperazin-2-one (301); (3S,6R)-3-isobutyl-6-(oxazol-5-yl)-4-((1R,2R)-2-phenylcyclopropane-carbonyl)piperazin-2-one (517); (3S,6S)-4-[(1R,2R)-2-(4-Chloro-phenyl)-cyclopropanecarbonyl]-6-cyclopentyl-3-isobutyl-piperazin-2-one (246); (3S,6S)-4-[(1R,2R)-2-(4-Chloro-2-fluoro-phenyl)-cyclopropanecarbonyl]-6-ethyl-3-isobutyl-piperazin-2-one (251); (3S,6S)-4-[(1R,2R)-2-(2,4-Difluoro-phenyl)-cyclopropanecarbonyl]-3,6-diisobutyl-piperazin-2-one (256); (3S,6S)-4-[(1R,2R)-2-(3,4-Difluoro-phenyl)-cyclopropanecarbonyl]-3-isobutyl-6-phenyl-piperazin-2-one (260); (3S,6S)-4-[(1R,2R)-2-(2,4-fluoro-phenyl)-cyclopropanecarbonyl]-3,6-diisobutyl-piperazin-2-one (298); (3S,6S)-4-((1R,2R)-2-(4-fluoro-phenyl)cyclopropanecarbonyl)-3-isobutyl-6-(tetrahydro-2H-pyran-4-yl)piperazin-2-one (476); (3S,6S)-4-[(1R,2R)-2-(4-Chloro-phenyl)-cyclopropanecarbonyl]-3,6-diisobutyl-piperazin-2-one (237); (3S,6R)-4-((1R,2R)-2-(4-fluorophenyl)cyclopropanecarbonyl)-3-propyl-6-(thiophen-2-yl)piperazin-2-one (495); (3S,6S)-3-isobutyl-6-phenyl-4-((1R,2R)-2-phenylcyclopropanecarbonyl)piperazin-2-one (234); (3S,6S)-4-[(1R,2R)-2-(4-Chloro-2-fluoro-phenyl)-cyclopropanecarbonyl]-3,6-diisobutyl-piperazin-2-one (250); (3S,6S)-4-[(1R,2R)-2-(4-Chloro-2-fluoro-phenyl)-cyclopropanecarbonyl]-3-isobutyl-6-phenyl-piperazin-2-one (255); (3S,6S)-4-[(1R,2R)-2-(4-Fluoro-phenyl)-cyclopropanecarbonyl]-6-cyclopropyl-methyl-3-isobutyl-piperazin-2-one (297); (3S,6S)-4-[(1R,2R)-2-(4-Chloro-phenyl)-cyclopropanecarbonyl]-6-cyclopropylmethyl-3-isobutyl-piperazin-2-one (243); (3S,6S)-4-[(1R,2R)-2-(4-Chloro-2-fluoro-phenyl)-cyclopropanecarbonyl]-6-cyclopentyl-3-isobutyl-piperazin-2-one (253); (3S,6S)-4-((1R,2R)-2-(4-bromophenyl)cyclopropanecarbonyl)-3,6-diisobutylpiperazin-2-one (329); (3S,6S)-6-(2-fluorophenyl)-3-isobutyl-4-((1R,2R)-2-phenylcyclopropanecarbonyl)-piperazin-2-one (357); (3S,6S)-6-Cyclohexyl-4-[(1R,2R)-2-(3,4-difluoro-phenyl)-cyclopropanecarbonyl]-3-isobutyl-piperazin-2-one (258); (3S,6S)-4-[(1R,2R)-2-(4-Bromo-phenyl)-cyclopropanecarbonyl]-3-isobutyl-6-propyl-piperazin-2-one (331); (3S,6S)-3-isobutyl-4-((1R,2R)-2-phenylcyclopropanecarbonyl)-6-propylpiperazin-2-one (230); (3S,6S)-4-[(1R,2R)-2-(3,4-Difluoro-phenyl)-cyclopropanecarbonyl]-3,6-diisobutyl-piperazin-2-one (238); (3S,6S)-4-[(1R,2R)-2-(3,4-Difluoro-phenyl)-cyclopropanecarbonyl]-3-isobutyl-6-propyl-piperazin-2-one (257); (3S,6S)-6-Cyclopentyl-4-[(1R,2R)-2-(3,4-difluoro-phenyl)-cyclopropanecarbonyl]-3-isobutyl-piperazin-2-one (259); (3S,6S)-4-[(1R,2R)-2-(4-Chloro-phenyl)-cyclopropanecarbonyl]-6-cyclohexyl-3-isobutyl-piperazin-2-one (245); (3S,6R)-4-((1R,2R)-2-(4-fluorophenyl)cyclopropanecarbonyl)-3-isobutyl-6-((methylthio)methyl)piperazin-2-one (299); (3S,6S)-4-[(1R,2R)-2-(4-bromo-phenyl)-cyclopropanecarbonyl]-3-isobutyl-6-phenyl-piperazin-2-one (333); (3S,6S)-4-[(1R,2R)-2-(4-Chloro-2-fluoro-phenyl)-cyclopropanecarbonyl]-3-isobutyl-6-isopropyl-piperazin-2-one (254); (3S,6S)-4-[(1R,2R)-2-(4-Chloro-2-fluoro-phenyl)-cyclopropanecarbonyl]-3-isobutyl-6-(1-propyl-butyl)-piperazin-2-one (252); (3S,6S)-6-cyclopentyl-3-isobutyl-4-((1R,2R)-2-phenylcyclopropanecarbonyl)-piperazin-2-one (232); (3S,6S)-4-[(1R,2R)-2-(4-tert-Butyl-phenyl)-cyclopropanecarbonyl]-3,6-diisobutyl-piperazin-2-one (249); (3S,6R)-4-((1R,2R)-2-(4-Chlorophenyl)cyclopropanecarbonyl)-3-isobutyl-6-((methylthio)methyl)piperazin-2-one (300); (3S,6S)-4-[(1R,2R)-2-(3,4-Difluoro-phenyl)-cyclopropanecarbonyl]-3-isobutyl-6-isopropyl-piperazin-2-one (309); (3S,6S)-3,6-diisobutyl-4-((1R,2R)-2-phenylcyclopropanecarbonyl)piperazin-2-one (70); (3S,6S)-6-cyclobutyl-4-((1R,2R)-2-(4-fluorophenyl)cyclopropanecarbonyl)-3-isobutylpiperazin-2-one (464); (3S,6S)-4-((1R,2R)-2-(4-(tert-butyl)phenyl)cyclopropanecarbonyl)-3-isobutyl-6-propyl-pylpiperazin-2-one (334); (3S,6S)-6-cyclohexyl-3-isobutyl-4-((1R,2R)-2-phenylcyclopropanecarbonyl)-piperazin-2-one (231); (3S,6S)-3-isobutyl-6-isopropyl-4-((1R,2R)-2-phenylcyclopropanecarbonyl)piperazin-2-one (233); (3S,6S)-4-((1R,2R)-2-(4-(tert-butyl)phenyl)cyclopropanecarbonyl)-6-cyclopentyl-3-isobutylpiperazin-2-one (310); (3S,6S)-3,6-Diisobutyl-4-[(1R,2R)-2-(4-methoxy-phenyl)-cyclopropanecarbonyl]-piperazin-2-one (239); (3S,6R)-6-(5-chlorothiophen-2-yl)-4-(5-(4-fluorophenyl)isoxazole-3-carbonyl)-3-isobutylpiperazin-2-one (490); (3S,6S)-3-neopentyl-6-phenyl-4-((1R,2R)-2-phenylcyclopropanecarbonyl)piperazin-2-one (344); (3R,6S)-4-((1R,2R)-2-(4-fluorophenyl)cyclopropanecarbonyl)-6-phenyl-3-(thiophen-2-yl)piperazin-2-one (469); (3S,6S)-4-((1R,2R)-2-(4-(tert-butyl)phenyl)cyclopropanecarbonyl)-3-isobutyl-6-phenylpiperazin-2-one (312); (3R,6S)-4-((1R,2R)-2-(4-fluorophenyl)cyclopropanecarbonyl)-3-((methylthio)-methyl)-6-phenylpiperazin-2-one (460); (3S,6S)-6-cyclopentyl-4-((1R,2R)-2-(4-bromophenyl)cyclopropanecarbonyl)-3-isobutylpiperazin-2-one (332); (3S,6S)-3-allyl-4-((1R,2R)-2-(4-fluorophenyl)cyclopropanecarbonyl)-6-phenyl-piperazin-2-one (462); (3S,6S)-6-(3-fluorophenyl)-3-isobutyl-4-((1R,2R)-2-phenylcyclopropanecarbonyl)-piperazin-2-one (369); (3S,6S)-6-(2-chlorophenyl)-3-isobutyl-4-((1R,2R)-2-phenylcyclopropanecarbonyl)-piperazin-2-one (354); (3S,6S)-4-((1R,2R)-2-(4-(tert-butyl)phenyl)cyclopropanecarbonyl)-3-isobutyl-6-isopropylpiperazin-2-one (311); (3S,6S)-6-(4-fluorophenyl)-3-isobutyl-4-((1R,2R)-2-phenylcyclopropanecarbonyl)-piperazin-2-one (347); (3S,6S)-3-isobutyl-4-((1R,2R)-2-phenylcyclopropanecarbonyl)-6-(o-tolyl)piperazin-2-one (363); (3S,6S)-6-phenyl-4-((1R,2R)-2-phenylcyclopropanecarbonyl)-3-propylpiperazin-2-one (399); (3S,6S)-3-isobutyl-4-((1R,2R)-2-phenylcyclopropanecarbonyl)-6-((E)-prop-1-en-1-yl)piperazin-2-one (422); (3S,6S)-4-((1R,2R)-2-(4-(tert-butyl)phenyl)cyclopropanecarbonyl)-6-cyclohexyl-3-isobutylpiperazin-2-one (328); (3S,6S)-3-((R)-sec-butyl)-6-phenyl-4-((1R,2R)-2-phenylcyclopropanecarbonyl)-piperazin-2-one (341); (3S,6S)-6-(4-chlorophenyl)-3-isobutyl-4-((1R,2R)-2-phenylcyclopropanecarbonyl)-piperazin-2-one (360); (3S,6S)-6-cyclopropyl-3-isobutyl-4-((1R,2R)-2-phenylcyclopropanecarbonyl)-piperazin-2-one (235); (3S,6S)-6-cyclohexyl-4-((1R,2R)-2-(2,4-difluorophenyl)cyclopropanecarbonyl)-3-isobutylpiperazin-2-one (335); (3S,6S)-3-(cyclopropylmethyl)-6-phenyl-4-((1R,2R)-2-phenylcyclopropanecarbonyl)-piperazin-2-one (338); (3S,6S)-3-isopropyl-6-phenyl-4-((1R,2R)-2-phenylcyclopropanecarbonyl)piperazin-2-one (402); (3S,6S)-4-[(1R,2R)-2-(2,4-difluoro-phenyl)-cyclopropanecarbonyl]-3-isobutyl-6-phenyl-piperazin-2-one (327); (3S,75)-3-isobutyl-7-phenyl-4-((1R,2R)-2-phenylcyclopropanecarbonyl)-1,4-diazepan-2-one (417); (3S,6S)-3-isobutyl-6-neopentyl-4-((1R,2R)-2phenylcyclopropanecarbonyl)piperazin-2-one (280); (3S,6S)-3,6-dineopentyl-4-((1R,2R)-2-phenylcyclopropanecarbonyl)-piperazin-2-one (281); (3S,6S)-4-[(1R,2R)-2-(2,4-difluoro-phenyl)-cyclopropanecarbonyl]-6-ethyl-3-isobutyl-piperazin-2-one (324); (3S,6S)-6-cyclopentyl-4-((1R,2R)-2-(2,4-difluorophenyl)cyclopropanecarbonyl)-3-isobutylpiperazin-2-one (325); (3S,6S)-6-cyclohexyl-4-((1R,2R)-2-(2,4-difluorophenyl)cyclopropanecarbonyl)-3-isobutylpiperazin-2-one (326); (3S,6S)-3-isobutyl-4-((1R,2R)-2-phenylcyclopropanecarbonyl)-6-(4-(trifluoro-methyl)phenyl)piperazin-2-one (364); (3S,6S)-3-isobutyl-6-phenyl-4-((1R,2R)-2-phenylcyclopropanecarbonyl)-1,4-diazepan-2-one (418); (3S,6S)-4-[(1S,2S)-2-(2,4-Difluoro-phenyl)-cyclopropanecarbonyl]-3,6-diisobutyl-piperazin-2-one (421); (3S,6R)-4-((1R,2R)-2-(4-fluorophenyl)cyclopropanecarbonyl)-3-isobutyl-6-(3-methylthiophen-2-yl)piperazin-2-one (438); (3S,6R)-4-((1R,2R)-2-(4-fluorophenyl)cyclopropanecarbonyl)-3-isobutyl-6-(tetrahy-dro-2H-pyran-4-yl)piperazin-2-one (475); (2R,5S)-5-isobutyl-2-phenyl-1-((1S,2S)-2-phenylcyclopropanecarbonyl)imidazolidin-4-one (530); (2R,5S)-5-isobutyl-2-phenyl-1-((1S,2S)-2-phenylcyclopropanecarbonyl)imidazolidin-4-one (531); (2R,5S)-1-((1S,2S)-2-(4-fluorophenyl)cyclopropanecarbonyl)-5-isobutyl-2-phenyl-imidazolidin-4-one (532); and (2R,5S)-1-((1S,2S)-2-(4-fluorophenyl)cyclopropane-carbonyl)-5-isobutyl-2-phenyl-imidazolidin-4-one (533).

In a fifth embodiment of compound (A) or its stereoisomer or its salt thereof where W is absent and the carbon atom of ˜C(—R₂)˜ is bound directly to the nitrogen atom of ˜N(—Z)˜ to form a compound represented by formula V:

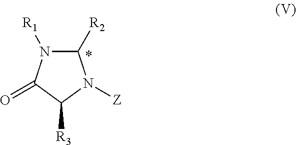

wherein
R₁ is selected from among hydrogen, an alkyl, an alkaryl, an acyl, an aryl, and a heteroaryl;
R₂ and R₃ are independently selected from among, hydrogen, an alkyl, allyl, halo, a cycloalkyl, an alkylene-cycloalkyl, an alkylenethioalkyl, an alkyleneoxoalkyl, an alkylene-cycloalkyl, an aryl, an alkaryl, a heteroaryl, an alkheteroaryl, COOH, COOalkyl, CONHalkyl, and CON(alkyl)₂; and
Z is selected from among ˜Q-Yₘ—B and ˜T-A-Xₙ—B
   where
   Q is —(CH₂)ₒ— (o is 0, 1, 2, or 3) or —C(O)—,
   Y is O, S, or NH and m is 0 or 1,
   T is —(CH₂)ₚ—(C(O))_{q}— (p is 0, 1, 2, or 3 and q is 0 or 1) or CHR₇, where R₇ is a C₁₋₆alkyl,
   X is CH₂, O, NH, or S, with n is 0 or 1
   A is selected from among —CR′═CR″—, —C≡C—, a cycloalkylene, an aryl, and a heteroaryl,
      where R′ and R″ are independently selected from among hydrogen, a C₁₋₆alkyl, and an aryl; and
   B is selected from among hydrogen, a C₁₋₆alkyl, a cycloalkyl, an aryl, a heteroaryl, and a fused ring moiety.

In a first aspect of the fifth embodiment Z is ˜Q-Yₘ—B, Q is —(CH₂)ₒ— (o is 0, 1, 2, or 3), and the compound of formula V is represented by formula (V-1)

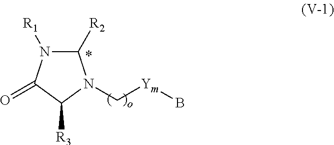

wherein
R₁ is selected from among hydrogen, an alkyl, an alkaryl, an acyl, an aryl, and a heteroaryl;
R₂ and R₃ are independently selected from among, hydrogen, an alkyl, allyl, halo, a cycloalkyl, an alkylene-cycloalkyl, an alkylenethioalkyl, an alkyleneoxoalkyl, an alkylene-cycloalkyl, an aryl, an alkaryl, a heteroaryl, an alkheteroaryl, COOH, COOalkyl, CONHalkyl, and CON(alkyl)₂;
Y is O, S, or NH and m is 0 or 1; and
B is selected from among hydrogen, a C₁₋₆alkyl, a cycloalkyl, an aryl, a heteroaryl, and a fused ring moiety.

In a second aspect of the fifth embodiment Z is ˜Q-Yₘ—B, Q is —(CH₂)ₒ— (o is 0, 1, 2, or 3), and the compound of formula V is represented by formula (V-1)

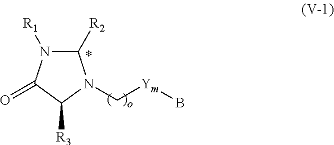

wherein
R₁ is selected from among hydrogen and a C₁₋₆alkyl;
R₂ and R₃ are independently selected from among, hydrogen, an alkyl, allyl, halo, a cycloalkyl, an alkylene-cycloalkyl, an alkylenethioalkyl, an alkyleneoxoalkyl, an alkylene-cycloalkyl, an aryl, an alkaryl, a heteroaryl, and an alkheteroaryl;
Y is O, S, or NH and m is 0 or 1; and
B is selected from among hydrogen, a C₁₋₆alkyl, a cycloalkyl, an aryl, a heteroaryl, and a fused ring moiety.

In a third aspect of the fifth embodiment Z is ˜Q-Yₘ—B, Q is —(CH₂)ₒ— (o is 0, 1, 2, or 3), and the compound of formula V is represented by formula (V-1)

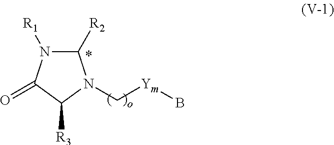

wherein
R₁ is selected from among hydrogen and a C₁₋₆alkyl;
R₂ and R₃ are independently selected from among, hydrogen, a C₁₋₆alkyl, allyl, a C₃₋₆cycloalkyl, a C₁₋₆alkyleneC₃₋₆cycloalkyl, a —(C₁₋₆-alkylene)oxo(C₁₋₆alkyl), a (C₁₋₆alkylene)thio(C₁₋₆alkyl), an aryl, a C₁₋₃alkaryl, a heteroaryl, and a C₁₋₆alkheteroaryl;
Y is O, S, or NH and m is 0 or 1; and
B is selected from among hydrogen, a C₁₋₆alkyl, a C₃₋₆cycloalkyl, an aryl, a heteroaryl, and a fused ring moiety.

In a sub-aspect of each of the first, second, and third aspects of the fifth embodiment, R₂ has a configuration as shown in formula (V-1′)

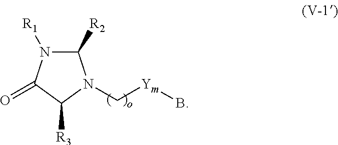

In a fourth aspect of the fifth embodiment Z is ˜Q-Yₘ—B, Q is —(CH₂)ₒ— (o is 0, 1, 2, or 3), and the compound of formula V is represented by formula (V-1)

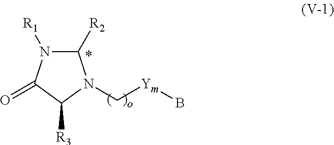

wherein
R₁ is hydrogen;
R₂ and R₃ are independently selected from among, hydrogen, a C₁₋₆alkyl, allyl, a C₃₋₆cycloalkyl, a C₁₋₆alkyleneC₃₋₆cycloalkyl, a —(C₁₋₆-alkylene)oxo(C₁₋₆alkyl), a (C₁₋₆alkylene)thio(C₁₋₆alkyl), an aryl, a C₁₋₃alkaryl, a heteroaryl, and a C₁₋₆alkheteroaryl;
Y is O, S, or NH and m is 0 or 1; and
B is selected from among hydrogen, a C₁₋₆alkyl, a C₃₋₆cycloalkyl, an aryl, a heteroaryl, and a fused ring moiety.

In a fifth aspect of the fifth embodiment Z is ˜Q-Yₘ—B, Q is —(CH₂)ₒ— (o is 0, 1, 2, or 3), and m is 0, and the compound of formula V is represented by formula (V-2)

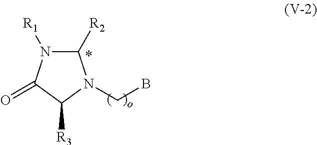

wherein
R₁ is selected from among hydrogen, an alkyl, an alkaryl, an acyl, an aryl, and a heteroaryl;
R₂ and R₃ are independently selected from among, hydrogen, an alkyl, allyl, halo, a cycloalkyl, an alkylene-cycloalkyl, an alkylenethioalkyl, an alkyleneoxoalkyl, an alkylene-cycloalkyl, an aryl, an alkaryl, a heteroaryl, an alkheteroaryl, COOH, COOalkyl, CONHalkyl, and CON(alkyl)₂;
Y is O, S, or NH and m is 0 or 1; and
B is selected from among hydrogen, a C₁₋₆alkyl, a cycloalkyl, an aryl, a heteroaryl, and a fused ring moiety.

In a sixth aspect of the fifth embodiment Z is ˜Q-Yₘ—B, Q is —(CH₂)ₒ— (o is 0, 1, 2, or 3), and m is 0, and the compound of formula V is represented by formula (V-2)

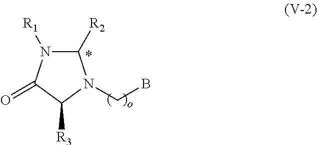

wherein
R₁ is selected from among hydrogen and a C₁₋₆alkyl;
R₂ and R₃ are independently selected from among, hydrogen, an alkyl, allyl, halo, a cycloalkyl, an alkylene-cycloalkyl, an alkylenethioalkyl, an alkyleneoxoalkyl, an alkylene-cycloalkyl, an aryl, an alkaryl, a heteroaryl, and an alkheteroaryl;
Y is O, S, or NH and m is 0 or 1; and
B is selected from among hydrogen, a C₁₋₆alkyl, a cycloalkyl, an aryl, a heteroaryl, and a fused ring moiety.

In a seventh aspect of the fifth embodiment Z is ˜Q-Yₘ—B, Q is —(CH₂)ₒ— (o is 0, 1, 2, or 3), and m is 0, and the compound of formula V is represented by formula (V-2)

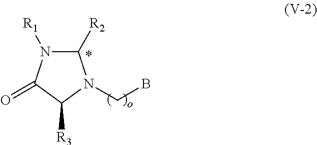

wherein
R₁ is selected from among hydrogen and a C₁₋₆alkyl;
R₂ and R₃ are independently selected from among, hydrogen, a C₁₋₆alkyl, allyl, a C₃₋₆cycloalkyl, a C₁₋₆alkyleneC₃₋₆cycloalkyl, a —(C₁₋₆-alkylene)oxo(C₁₋₆alkyl), a (C₁₋₆alkylene)thio(C₁₋₆alkyl), an aryl, a C₁₋₃alkaryl, a heteroaryl, and a C₁₋₆alkheteroaryl;
Y is O, S, or NH and m is 0 or 1; and
B is selected from among hydrogen, a C₁₋₆alkyl, a C₃₋₆cycloalkyl, an aryl, a heteroaryl, and a fused ring moiety.

In an eighth aspect of the fifth embodiment Z is ˜Q-Yₘ—B, Q is —(CH₂)ₒ— (o is 0, 1, 2, or 3), and m is 0, and the compound of formula V is represented by formula (V-2)

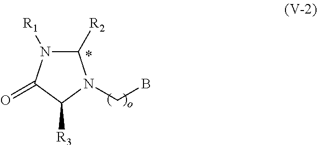

wherein
R₁ is hydrogen;
R₂ and R₃ are independently selected from among, hydrogen, a C₁₋₆alkyl, allyl, a C₃₋₆cycloalkyl, a C₁₋₆alkyleneC₃₋₆cycloalkyl, a —(C₁₋₆-alkylene)oxo(C₁₋₆alkyl), a (C₁₋₆alkylene)thio(C₁₋₆alkyl), an aryl, a C₁₋₃alkaryl, a heteroaryl, and a C₁₋₆alkheteroaryl;
Y is O, S, or NH and m is 0 or 1; and
B is selected from among hydrogen, a C₁₋₆alkyl, a C₃₋₆cycloalkyl, an aryl, a heteroaryl, and a fused ring moiety.

In a sub-aspect of each of the fourth, fifth, sixth, seventh, and eighth aspects of the fifth embodiment, R₂ has a configuration as shown in formula (V-2′)

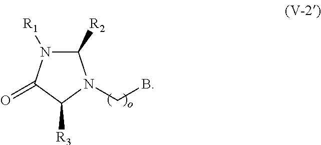

In a ninth aspect of the fifth embodiment Z is ˜Q-Yₘ—B, Q is —C(O)—, and the compound of formula V is represented by formula (V-3)

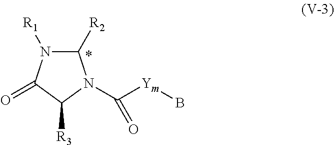

wherein
R₁ is selected from among hydrogen, an alkyl, an alkaryl, an acyl, an aryl, and a heteroaryl;
R₂ and R₃ are independently selected from among, hydrogen, an alkyl, allyl, halo, a cycloalkyl, an alkylene-cycloalkyl, an alkylenethioalkyl, an alkyleneoxoalkyl, an alkylene-cycloalkyl, an aryl, an alkaryl, a heteroaryl, an alkheteroaryl, COOH, COOalkyl, CONHalkyl, and CON(alkyl)₂;
Y is CH₂, O, S, or NH and m is 0 or 1; and
B is selected from among hydrogen, a C₁₋₆alkyl, a cycloalkyl, an aryl, a heteroaryl, and a fused ring moiety.

In a tenth aspect of the fifth embodiment Z is ˜Q-Yₘ—B, Q is —C(O)—, and the compound of formula V is represented by formula (V-3)

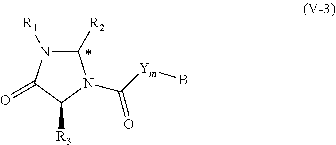

wherein
R₁ is selected from among hydrogen and a C₁₋₆alkyl;
R₂ and R₃ are independently selected from among, hydrogen, an alkyl, allyl, halo, a cycloalkyl, an alkylene-cycloalkyl, an alkylenethioalkyl, an alkyleneoxoalkyl, an alkylene-cycloalkyl, an aryl, an alkaryl, a heteroaryl, and an alkheteroaryl;
Y is CH₂, O, S, or NH and m is 0 or 1; and
B is selected from among hydrogen, a C₁₋₆alkyl, a cycloalkyl, an aryl, a heteroaryl, and a fused ring moiety.

In an eleventh aspect of the fifth embodiment Z is ˜Q-Yₘ—B, Q is —C(O)—, and the compound of formula V is represented by formula (V-3)

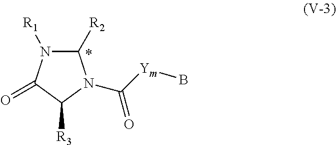

wherein
R₁ is selected from among hydrogen and a C₁₋₆alkyl;
R₂ and R₃ are independently selected from among, hydrogen, a C₁₋₆alkyl, allyl, a C₃₋₆cycloalkyl, a C₁₋₆alkyleneC₃₋₆cycloalkyl, a —(C₁₋₆-alkylene)oxo(C₁₋₆alkyl), a (C₁₋₆alkylene)thio(C₁₋₆alkyl), an aryl, a C₁₋₃alkaryl, a heteroaryl, and a C₁₋₆alkheteroaryl;
Y is CH₂, O, S, or NH and m is 0 or 1; and
B is selected from among hydrogen, a C₁₋₆alkyl, a C₃₋₆cycloalkyl, an aryl, a heteroaryl, and a fused ring moiety.

In a twelfth aspect of the fifth embodiment Z is ˜Q-Yₘ—B, Q is —C(O)—, and the compound of formula V is represented by formula (V-3)

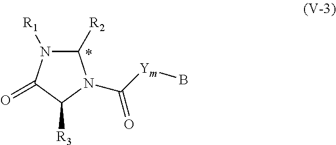

wherein
R₁ is hydrogen;
R₂ and R₃ are independently selected from among, hydrogen, a C₁₋₆alkyl, allyl, a C₃₋₆cycloalkyl, a C₁₋₆alkyleneC₃₋₆cycloalkyl, a —(C₁₋₆-alkylene)oxo(C₁₋₆alkyl), a (C₁₋₆alkylene)thio(C₁₋₆alkyl), an aryl, a C₁₋₃alkaryl, a heteroaryl, and a C₁₋₆alkheteroaryl;
Y is CH₂, O, S, or NH and m is 0 or 1; and
B is selected from among hydrogen, a C₁₋₆alkyl, a C₃₋₆cycloalkyl, an aryl, a heteroaryl, and a fused ring moiety.

In a sub-aspect of each of the ninth, tenth, and eleventh aspects of the fifth embodiment, R₂ has a configuration as shown in formula (V-3′)

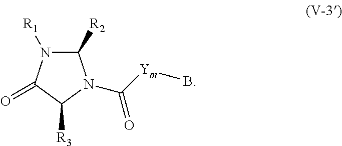

In a 13th aspect of the fifth embodiment Z is ˜Q-Yₘ—B, Q is —C(O)—, m is 0, and the compound of formula V is represented by formula (V-4)

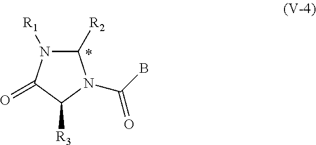

wherein
R₁ is selected from among hydrogen, an alkyl, an alkaryl, an acyl, an aryl, and a heteroaryl;
R₂ and R₃ are independently selected from among, hydrogen, an alkyl, allyl, halo, a cycloalkyl, an alkylene-cycloalkyl, an alkylenethioalkyl, an alkyleneoxoalkyl, an alkylene-cycloalkyl, an aryl, an alkaryl, a heteroaryl, an alkheteroaryl, COOH, COOalkyl, CONHalkyl, and CON(alkyl)₂; and
B is selected from among hydrogen, a C₁₋₆alkyl, a cycloalkyl, an aryl, a heteroaryl, and a fused ring moiety.

In a 14th aspect of the fifth embodiment Z is ˜Q-Yₘ—B, Q is —C(O)—, m is 0, and the compound of formula V is represented by formula (V-4)

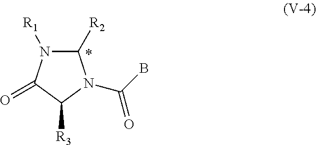

wherein
R₁ is selected from among hydrogen and a C₁₋₆alkyl;
R₂ and R₃ are independently selected from among, hydrogen, an alkyl, allyl, halo, a cycloalkyl, an alkylene-cycloalkyl, an alkylenethioalkyl, an alkyleneoxoalkyl, an alkylene-cycloalkyl, an aryl, an alkaryl, a heteroaryl, and an alkheteroaryl; and
B is selected from among hydrogen, a C₁₋₆alkyl, a cycloalkyl, an aryl, a heteroaryl, and a fused ring moiety.

In a 15th aspect of the fifth embodiment Z is ˜Q-Yₘ—B, Q is —C(O)—, m is 0, and the compound of formula V is represented by formula (V-4)

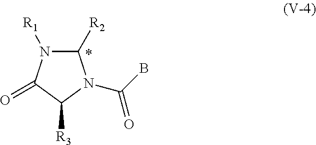

wherein
R₁ is selected from among hydrogen and a C₁₋₆alkyl;
R₂ and R₃ are independently selected from among, hydrogen, a C₁₋₆alkyl, allyl, a C₃₋₆cycloalkyl, a C₁₋₆alkyleneC₃₋₆cycloalkyl, a —(C₁₋₆-alkylene)oxo(C₁₋₆alkyl), a (C₁₋₆alkylene)thio(C₁₋₆alkyl), an aryl, a C₁₋₃alkaryl, a heteroaryl, and a C₁₋₆alkheteroaryl; and
B is selected from among hydrogen, a C₁₋₆alkyl, a C₃₋₆cycloalkyl, an aryl, a heteroaryl, and a fused ring moiety.

In a 16th aspect of the fifth embodiment Z is ˜Q-Yₘ—B, Q is —C(O)—, m is 0, and the compound of formula V is represented by formula (V-4)

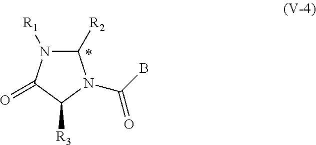

wherein
R₁ is hydrogen;
R₂ and R₃ are independently selected from among, hydrogen, a C₁₋₆alkyl, allyl, a C₃₋₆cycloalkyl, a C₁₋₆alkyleneC₃₋₆cycloalkyl, a —(C₁₋₆-alkylene)oxo(C₁₋₆alkyl), a (C₁₋₆alkylene)thio(C₁₋₆alkyl), an aryl, a C₁₋₃alkaryl, a heteroaryl, and a C₁₋₆alkheteroaryl; and
B is selected from among hydrogen, a C₁₋₆alkyl, a C₃₋₆cycloalkyl, an aryl, a heteroaryl, and a fused ring moiety.

In a sub-aspect of each of the 12th, 13th, 14th, 15th, and 16th aspects of the fifth embodiment, R₂ has a configuration as shown in formula (V-4′)

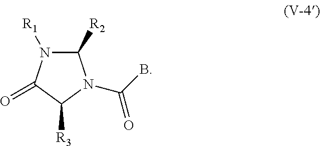

In a 17th aspect of the fifth embodiment Z is ˜T-A-Xₙ—B, T is —(CH₂)ₚ—(C(O))_{q}— (p is 0, q is 1, and n is 0), and the compound of formula V is represented by formula (V-5)

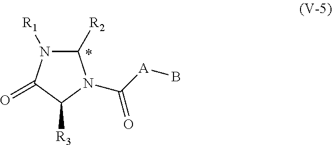

wherein
R₁ is selected from among hydrogen, an alkyl, an alkaryl, an acyl, an aryl, and a heteroaryl;
R₂ and R₃ are independently selected from among hydrogen, an alkyl, allyl, halo, a cycloalkyl, an alkylene-cycloalkyl, an alkylenethioalkyl, an alkyleneoxoalkyl, an alkylene-cycloalkyl, an aryl, an alkaryl, a heteroaryl, an alkheteroaryl, COOH, COOalkyl, CONHalkyl, and CON(alkyl)₂;
A is selected from among —CR′═CR″—, —C≡C—, a cycloalkylene, an arylene, and a heteroarylene,
   where R′ and R″ are independently selected from among hydrogen, a C₁₋₆alkyl, and an aryl; and
B is selected from among hydrogen, a C₁₋₆alkyl, a cycloalkyl, an aryl, a heteroaryl, and a fused ring moiety.

In an 18th aspect of the fifth embodiment Z is ˜T-A-Xₙ—B, T is —(CH₂)ₚ—(C(O))_{q}— (p is 0, q is 1, and n is 0), and the compound of formula V is represented by formula (V-5)

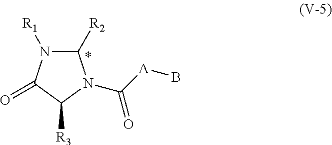

wherein
R₁ is selected from among hydrogen, an alkyl, an alkaryl, an acyl, an aryl, and a heteroaryl;
R₂ and R₃ are independently selected from among hydrogen, an alkyl, allyl, halo, a cycloalkyl, an alkylene-cycloalkyl, an alkylenethioalkyl, an alkyleneoxoalkyl, an alkylene-cycloalkyl, an aryl, an alkaryl, a heteroaryl, an alkheteroaryl;
A is selected from among —CR′═CR″—, —C≡C—, a cycloalkylene, an arylene, and a heteroarylene,
   where R′ and R″ are independently selected from among hydrogen, a C₁₋₆alkyl, and an aryl; and
B is selected from among hydrogen, a C₁₋₆alkyl, a cycloalkyl, an aryl, a heteroaryl, and a fused ring moiety.

In a 19th aspect of the fifth embodiment Z is ˜T-A-Xₙ—B, T is —(CH₂)ₚ—(C(O))_{q}— (p is 0, q is 1, and n is 0), and the compound of formula V is represented by formula (V-5)

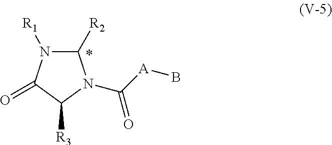

wherein
R₁ is selected from among hydrogen, a C₁₋₆alkyl and a C₁₋₃alkaryl;
R₂ and R₃ are independently selected from among hydrogen, a C₁₋₆alkyl, allyl, a C₃₋₆cycloalkyl, a C₁₋₆alkylene-C₃₋₆cycloalkyl, a C₁₋₆alkylenethioC₁₋₆alkyl, a C₁₋₆alkyleneoxoC₁₋₆alkyl, a C₁₋₆alkylene-C₃₋₆cycloalkyl, an aryl, a C₁₋₃alkaryl, a heteroaryl, a C₁₋₃alkheteroaryl;
A is selected from among —CR′═CR″—, —C≡C—, a C₃₋₆cycloalkylene, an arylene, and a heteroarylene,
   where R′ and R″ are independently selected from among hydrogen, a C₁₋₆alkyl, and an aryl; and
B is selected from among hydrogen, a C₁₋₆alkyl, a C₃₋₆cycloalkyl, an aryl, a heteroaryl, and a fused ring moiety.

In a 20th aspect of the fifth embodiment Z is ˜T-A-Xₙ—B, T is —(CH₂)ₚ—(C(O))_{q}— (p is 0, q is 1, and n is 0), and the compound of formula V is represented by formula (V-5)

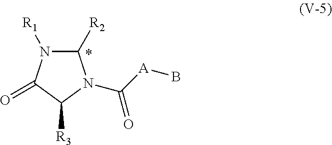

wherein
R₁ is hydrogen or a C₁₋₆alkyl;
R₂ and R₃ are independently selected from among hydrogen, a C₁₋₆alkyl, a C₃₋₆cycloalkyl, a C₁₋₆alkylene-C₃₋₆cycloalkyl, a C₁₋₆alkylenethioC₁₋₆alkyl, a C₁₋₆alkyleneoxoC₁₋₆alkyl, a C₁₋₆alkylene-C₃₋₆cycloalkyl, an aryl, an C₁₋₃alkaryl, a heteroaryl, a C₁₋₃alkheteroaryl;
A is selected from among —CR′═CR″—, —C≡C—, a C₃₋₆cycloalkylene, an arylene, and a heteroarylene,
   where R′ and R″ are independently selected from among hydrogen and a C₁₋₆alkyl; and
B is selected from among hydrogen, a C₁₋₆alkyl, a C₃₋₆cycloalkyl, an aryl, a heteroaryl, and a fused ring moiety.

In a 21st aspect of the fifth embodiment Z is ˜T-A-Xₙ—B, T is —(CH₂)ₚ—(C(O))_{q}— (p is 0, q is 1, and n is 0), and the compound of formula V is represented by formula (V-5)

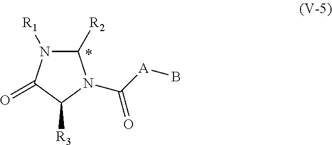

wherein
R₁ is hydrogen or a C₁₋₆alkyl;
R₂ and R₃ are independently selected from among hydrogen, a C₁₋₆alkyl, a C₃₋₆cycloalkyl, a C₁₋₆alkylene-C₃₋₆cycloalkyl, a C₁₋₆alkylenethioC₁₋₆alkyl, a C₁₋₆alkyleneoxoC₁₋₆alkyl, a C₁₋₆alkylene-C₃₋₆cycloalkyl, an aryl, a C₁₋₃alkaryl, a heteroaryl, and a C₁₋₃alkheteroaryl;
A is a heteroarylene; and
B is selected from among hydrogen, a C₁₋₆alkyl, a C₃₋₆cycloalkyl, an aryl, a heteroaryl, and a fused ring moiety.

In a 22nd aspect of the fifth embodiment Z is ˜T-A-Xₙ—B, T is —(CH₂)ₚ—(C(O))_{q}— (p is 0, q is 1, and n is 0), and the compound of formula V is represented by formula (V-5)

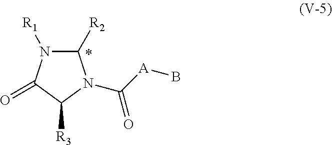

wherein
R₁ is hydrogen or a C₁₋₆alkyl;
R₂ and R₃ are independently selected from among hydrogen, a C₁₋₆alkyl, a C₃₋₆cycloalkyl, a C₁₋₆alkylene-C₃₋₆cycloalkyl, a C₁₋₆alkylenethioC₁₋₆alkyl, a C₁₋₆alkyleneoxoC₁₋₆alkyl, a C₁₋₆alkylene-C₃₋₆cycloalkyl, an aryl, a C₁₋₃alkaryl, a heteroaryl, and a C₁₋₃alkheteroaryl;
A is selected from an N-ring, as defined herein; and
B is selected from among hydrogen, a C₁₋₆alkyl, a C₃₋₆cycloalkyl, an aryl, a heteroaryl, and a fused ring moiety.

In a 23rd aspect of the fifth embodiment Z is ˜T-A-Xₙ—B, T is —(CH₂)ₚ—(C(O))_{q}— (p is 0, q is 1, and n is 0), and the compound of formula V is represented by formula (V-5)

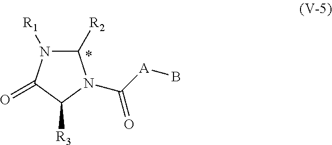

wherein
R₁ is hydrogen or a C₁₋₆alkyl;
R₂ and R₃ are independently selected from among hydrogen, a C₁₋₆alkyl, a C₃₋₆cycloalkyl, a C₁₋₆alkylene-C₃₋₆cycloalkyl, a C₁₋₆alkylenethioC₁₋₆alkyl, a C₁₋₆alkyleneoxoC₁₋₆alkyl, a C₁₋₆alkylene-C₃₋₆cycloalkyl, an aryl, a C₁₋₃alkaryl, a heteroaryl, a C₁₋₃alkheteroaryl;
A is selected from an A′-ring, as defined herein; and
B is selected from a B′-ring, as defined herein.

In a 24th aspect of the fifth embodiment Z is ˜T-A-Xₙ—B, T is —(CH₂)ₚ—(C(O))_{q}— (p is 0, q is 1, and n is 0), and the compound of formula V is represented by formula (V-5)

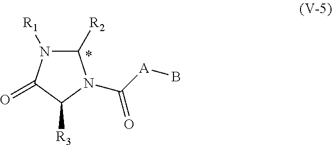

wherein
R₁ is hydrogen;
R₂ is selected from among an R₂-substituent, as defined herein;
R₃ is selected from among CH₃, CH₂CH₃, CH₂CH₂CH₃, CH₂OCH₃, CH₂SCH₃, CH(CH₃)₂, CH(CH₃)(CH₂CH₃), C(CH₃)₃, CH₂CH(CH₃)₂, CH₂C(CH₃)₃, CH═CHCH₃, CH═CH, CH₂cPr, CH₂cBu, CH₂cPn, CH₂cHx, cPr, cBu, cPn, cHx, and CH₂Ph;
each of R₄, R₅, and R₆ is hydrogen;
A is selected from an A′-ring, as defined herein; and
B is selected from a B′-ring, as defined herein.

In a sub-aspect of each of the 17th, 18th, 19th, 20th, 21st, 22nd, 23rd, and 24th aspects of the fifth embodiment, R₂ has a configuration as shown in formula (V-5′)

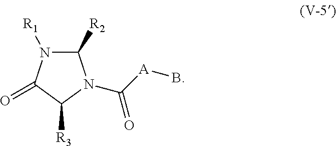

### Dosage, Administration, and Use

In the embodiments of this section, the expression “compound (A)″” is meant to encompass a compound or its stereoisomer or its salt thereof represented by formula A in the Summary or compounds described in each of the respective embodiments, aspects and sub-aspects of the embodiments, or the compounds recited in the examples.

A sixth embodiment is directed to a composition comprising compound (A).

A first aspect of the sixth embodiment is directed to a composition for treating a subject infected with any one of hepatitis C virus, hepatitis B virus, Hepatitis A virus, West Nile virus, yellow fever virus, dengue virus, rhinovirus, polio virus, bovine viral diarrhea virus, Japanese encephalitis virus, or those viruses belonging to the groups of Pestiviruses, hepaciviruses, or flavaviruses, said composition comprising an effective amount of compound (A).

A second aspect of the sixth embodiment is directed to a composition for treating a subject infected with a hepatitis C virus, which comprises an effective amount of compound (A) and optionally a pharmaceutically acceptable medium.

A third aspect of the sixth embodiment is directed to a composition for treating a subject infected with any one of a hepatitis B virus, a Hepatitis A virus, a West Nile virus, a yellow fever virus, a dengue virus, a rhinovirus, polio virus, a bovine viral diarrhea virus, and a Japanese encephalitis virus, which comprises an effective amount of compound (A) and a pharmaceutically acceptable medium.

A fourth aspect of the sixth embodiment is directed to a composition for treating a subject infected with a virus from any one of viruses belonging to the groups of Pestiviruses, hepaciviruses, or flavaviruses, which comprises an effective amount of compound (A) and a pharmaceutically acceptable medium.

Compound (A) may be independently formulated in a wide variety of oral administration dosage forms and carriers. Oral administration can be in the form of tablets, coated tablets, hard and soft gelatin capsules, solutions, emulsions, syrups, or suspensions. Compound (A) is efficacious when administered by suppository administration, among other routes of administration. The most convenient manner of administration is generally oral using a convenient daily dosing regimen which can be adjusted according to the severity of the disease and the patient's response to the antiviral medication.

Compound (A) together with one or more conventional excipients, carriers, or diluents, may be placed into the form of pharmaceutical compositions and unit dosages. The pharmaceutical compositions and unit dosage forms may be comprised of conventional ingredients in conventional proportions, with or without additional active compounds and the unit dosage forms may contain any suitable effective amount of the active ingredient commensurate with the intended daily dosage range to be employed. The pharmaceutical compositions may be employed as solids, such as tablets or filled capsules, semisolids, powders, sustained release formulations, or liquids such as suspensions, emulsions, or filled capsules for oral use; or in the form of suppositories for rectal or vaginal administration. A typical preparation will contain from about 5% to about 95% active compound or compounds (w/w).

As noted above, the term “effective amount” as used herein means an amount required to reduce symptoms of the disease in a subject. The dose will be adjusted to the individual requirements in each particular case. That dosage can vary within wide limits depending upon numerous factors such as the severity of the disease to be treated, the age and general health condition of the patient, other medicaments with which the patient is being treated, the route and form of administration and the preferences and experience of the medical practitioner involved. For oral administration, a daily dosage of between about 0.001 and about 10 g, including all values in between, such as 0.001, 0.0025, 0.005, 0.0075, 0.01, 0.025, 0.050, 0.075, 0.1, 0.125, 0.150, 0.175, 0.2, 0.25, 0.3, 0.4, 0.5, 0.6, 0.7 0.75, 0.8, 0.9, 1, 1.5, 2, 2.5, 3, 3.5, 4, 4.5, 5, 5.5, 6, 6.5, 7, 7.5, 8, 8.5, 9, and 9.5, per day should be appropriate in monotherapy and/or in combination therapy. A particular daily dosage is between about 0.01 and about 1 g per day, including all incremental values of 0.01 g (i.e., 10 mg) in between, a preferred daily dosage about 0.01 and about 0.8 g per day, more preferably about 0.01 and about 0.6 g per day, and most preferably about 0.01 and about 0.25 g per day, each of which including all incremental values of 0.01 g in between. Generally, treatment is initiated with a large initial “loading dose” to rapidly reduce or eliminate the virus following by a decreasing the dose to a level sufficient to prevent resurgence of the infection. One of ordinary skill in treating diseases described herein will be able, without undue experimentation and in reliance on knowledge, experience and the disclosures of this application, to ascertain a effective amount of the compound disclosed herein for a given disease and patient.

Compound (A) can be administered alone but will generally be administered in admixture with one or more suitable pharmaceutical excipients, diluents or carriers selected with regard to the intended route of administration and standard pharmaceutical practice.

Solid form preparations include, for example, powders, tablets, pills, capsules, suppositories, and dispersible granules. A solid carrier may be one or more substances which may also act as diluents, flavoring agents, solubilizers, lubricants, suspending agents, binders, preservatives, tablet disintegrating agents, or an encapsulating material. In powders, the carrier generally is a finely divided solid which is a mixture with the finely divided active component. In tablets, the active component generally is mixed with the carrier having the necessary binding capacity in suitable proportions and compacted in the shape and size desired. Suitable carriers include but are not limited to magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. Solid form preparations may contain, in addition to the active component, colorants, flavors, stabilizers, buffers, artificial and natural sweeteners, dispersants, thickeners, solubilizing agents, and the like. Examples of solid formulations are exemplified in EP 0524579; US 2002/0142050; US 2004/0224917; US 2005/0048116; US 2005/0058710; US 2006/0034937; US 2006/0057196; US 2006/0188570; US 2007/0026073; US 2007/0059360; US 2007/0077295; US 2007/0099902; US 2008/0014228; U.S. Pat. No. 6,267,985; U.S. Pat. No. 6,294,192; U.S. Pat. No. 6,383,471; U.S. Pat. No. 6,395,300; U.S. Pat. No. 6,569,463; U.S. Pat. No. 6,635,278; U.S. Pat. No. 6,645,528; U.S. Pat. No. 6,923,988; U.S. Pat. No. 6,932,983; U.S. Pat. No. 7,060,294; and U.S. Pat. No. 7,462,608.

Liquid formulations also are suitable for oral administration include liquid formulation including emulsions, syrups, elixirs and aqueous suspensions. These include solid form preparations which are intended to be converted to liquid form preparations shortly before use. Examples of liquid formulation are exemplified in U.S. Pat. Nos. 3,994,974; 5,695,784; and 6,977,257. Emulsions may be prepared in solutions, for example, in aqueous propylene glycol solutions or may contain emulsifying agents such as lecithin, sorbitan monooleate, or acacia. Aqueous suspensions can be prepared by dispersing the finely divided active component in water with viscous material, such as natural or synthetic gums, resins, methylcellulose, sodium carboxymethylcellulose, and other well known suspending agents.

Compound (A) may be independently formulated for administration as suppositories. A low melting wax, such as a mixture of fatty acid glycerides or cocoa butter is first melted and the active component is dispersed homogeneously, for example, by stirring. The molten homogeneous mixture is then poured into convenient sized molds, allowed to cool, and to solidify.

Compound (A) may be independently formulated for vaginal administration. Pessaries, tampons, creams, gels, pastes, foams or sprays containing in addition to the active ingredient such carriers as are known in the art to be appropriate. Certain of these formulations may also be used in conjunction with a condom with or without a spermicidal agent.

Suitable formulations along with pharmaceutical carriers, diluents and excipients are described in Remington: The Science and Practice of Pharmacy 1995, edited by E. W. Martin, Mack Publishing Company, 19th edition, Easton, Pa. A skilled formulation scientist may modify the formulations within the teachings of the specification to provide numerous formulations for a particular route of administration without rendering compositions containing the compounds contemplated herein unstable or compromising their therapeutic activity.

Additionally, compound (A) may be independently formulated in conjunction with liposomes, micelles, or complexed to or entrapped in a protein matrix, such as albumin, or associated with a cyclodextrin. As to liposomes, it is contemplated that compound (A) can be formulated in a manner as disclosed in U.S. Pat. Nos. 4,797,285; 5,013,556; 5,077,056; 5,077,057; 5,154,930; 5,192,549; 5,213,804; 5,225,212; 5,277,914; 5,316,771; 5,376,380; 5,549,910; 5,567,434; 5,736,155; 5,827,533; 5,882,679; 5,891,468; 6,060,080; 6,132,763; 6,143,321; 6,180,134; 6,200,598; 6,214,375; 6,224,903; 6,296,870; 6,653,455; 6,680,068; 6,726,925; 7,060,689; and 7,070,801. As to micelles, it is contemplated that compound (A) can be formulated in a manner as disclosed in U.S. Pat. Nos. 5,145,684 and 5,091,188. As to a protein matrix, it is contemplated that compound (A) can be complexed to or entrapped in a protein matrix as disclosed in any one of U.S. Pat. Nos. 5,439,686; 5,498,421; 6,096,331; 6,506,405; 6,537,579; 6,749,868; 6,753,006; and 7,820,788. As to a cyclodextrin, a sulfoalkylether-β-cyclodextrin is preferably used, and more preferably a sulfobutylether-β-cyclodextrin having an average substitution of about seven, i.e., SBE7-β-cyclodextrin, as described in U.S. Pat. No. 5,376,645.

An seventh embodiment is directed to a use of compound (A) for the manufacture of a medicament of the treatment of any condition the result of an infection by any one of the following viral agents: hepatitis C virus, West Nile virus, yellow fever virus, degue virus, rhinovirus, polio virus, hepatitis A virus, bovine viral diarrhea virus and Japanese encephalitis virus.

A first aspect of the seventh embodiment is directed to a use of compound (A) for the manufacture of a medicament of the treatment of a hepatitis C virus.

A second aspect of the seventh embodiment is directed to a use of compound (A) for the manufacture of a medicament of the treatment of any condition the result of an infection by any one of the following viral agents: a West Nile virus, a yellow fever virus, a degue virus, a rhinovirus, a polio virus, a hepatitis A virus, a bovine viral diarrhea virus, and a Japanese encephalitis virus.

A third aspect of the seventh embodiment is directed to a use of compound (A) for the manufacture of a medicament of the treatment of any condition the result of an infection by a viral agent from any one of viruses belonging to the groups of Pestiviruses, hepaciviruses, or flavaviruses.

As noted above, the term “medicament” means a substance used in a method of treatment and/or prophylaxis of a subject in need thereof, wherein the substance includes, but is not limited to, a composition, a formulation, a dosage form, and the like, comprising compound (A). It is contemplated that the use of any of compound (A) for the manufacture of a medicament for the treatment of any of the antiviral conditions disclosed herein, either alone or in combination with another compound disclosed herein. A medicament includes, but is not limited to, any one of the compositions contemplated by the fifth embodiment disclosed herein.

An eighth embodiment is directed to a method of treating a subject infected with any one of a hepatitis C virus, a West Nile virus, a yellow fever virus, a degue virus, a rhinovirus, a polio virus, a hepatitis A virus, a bovine viral diarrhea virus, a Japanese encephalitis virus or those viruses belonging to the groups of Pestiviruses, hepaciviruses, or flavaviruses, said method comprising administering an effective amount of compound (A) to the subject.

A first aspect of the eighth embodiment is directed to a method of treating a subject infected with a hepatitis C virus, said method comprising administering an effective amount of compound (A) to the subject.

A second aspect of the eighth embodiment is directed to a method of treating a subject injected with any one of a West Nile virus, a yellow fever virus, a degue virus, a rhinovirus, a polio virus, a hepatitis A virus, a bovine viral diarrhea virus, a Japanese encephalitis virus or those viruses belonging to the groups of Pestiviruses, hepaciviruses, or flavaviruses, said method comprising administering an effective amount of compound (A) to the subject.

It is intended that a subject in need thereof is one that has any condition the result of an infection by any of the viral agents disclosed herein, which includes, but is not limited to, a hepatitis C virus, a West Nile virus, a yellow fever virus, a degue virus, a rhinovirus, a polio virus, a hepatitis A virus, a bovine viral diarrhea virus or a Japanese encephalitis virus; flaviviridae viruses or pestiviruses or hepaciviruses or a viral agent causing symptoms equivalent or comparable to any of the above-listed viruses.

As noted above, the term “subject” means a mammal, which includes, but is not limited to, cattle, pigs, sheep, buffalo, llama, dogs, cats, and humans, preferably the subject is a human. It is contemplated that in the method of treating a subject thereof of the eighth embodiment can be any of the compounds contemplated herein, either alone or in combination with another compound disclosed herein.

Therapeutic efficacy can be ascertained from tests of liver function including, but not limited to protein levels such as serum proteins (e.g., albumin, clotting factors, alkaline phosphatase, aminotransferases (e.g., alanine transaminase, aspartate transaminase), 5′-nucleosidase, γ-glutaminyltranspeptidase, etc.), synthesis of bilirubin, synthesis of cholesterol, and synthesis of bile acids; a liver metabolic function, including, but not limited to, carbohydrate metabolism, amino acid and ammonia metabolism. Alternatively the therapeutic effectiveness may be monitored by measuring HCV-RNA. The results of these tests will allow the dose to be optimized.

In the case of HCV, therapeutic efficacy can be ascertained from the amount of HCV RNA in the subject, such as a human. A sustained virologic response (SVR) for a human receiving a drug treatment regimen is free of HCV RNA (LOD<about 15 IU/mL) for at least 12 weeks, post treatment as measured in accordance with the assay methodology described in US 2010/0226885 (U.S. Ser. No. 12/376,180). SVR is also described in detail by Dr. Steven L. Flamm in the Journal of the American Medical Association, Vol. 289, No. 18, pp. 2413 to 2417 (2003).

A third aspect of the eighth embodiment is directed to a method of treating a subject infected with hepatitis C virus, said method comprising administering to the subject an effective amount of compound (A) and an effective amount of another antiviral agent; wherein the administration is concurrent or alternative. It is understood that the time between alternative administration can range between 1-24 hours, which includes any sub-range in between including, 2, 3, 4, 5, 6, 7, 8, 9, 10, 11, 12, 13, 14, 15, 16, 17, 18, 19, 20, 21, 22, and 23 hours. It will be understood that the effective amount of compound (A) and the effective amount of another antiviral agent can be formulated in the same dosage form or formulated in separate dosage forms.

When compound (A) is administered in combination with another antiviral agent the activity may be increased over the activity exhibited for compound (A) alone. When the treatment is combination therapy, such administration may be concurrent or sequential with respect to that of the nucleoside derivatives. “Concurrent administration” as used herein thus includes administration of the agents at the same time or at different times. Administration of two or more agents at the same time can be achieved by a single formulation containing two or more active ingredients or by substantially simultaneous administration of two or more dosage forms with a single active agent.

It will be understood that references herein to treatment extend to prophylaxis as well as to the treatment of existing conditions.

Examples of “another antiviral agent” include, but are not limited to: HCV NS3 protease inhibitors (see EP 1881001, US 2003187018, US 2005267018, WO 2003006490, WO 200364456, WO 2004094452, WO 2005028502, WO 2005037214, WO 2005095403, WO 2007014920, WO 2007014921, WO 2007014922, WO 2007014925, WO 2007014926, WO 2007015824, WO 2008010921, and WO 2008010921); HCV NS5B Inhibitors (see US 2004229840, US 2005154056, US 2005-98125, US 20060194749, US 20060241064, US 20060293306, US 2006040890, US 2006040927, US 2006166964, US 2007275947, U.S. Pat. No. 6,784,166, US20072759300, WO 2002057287, WO 2002057425, WO 2003010141, WO 2003037895, WO 2003105770, WO 2004000858, WO 2004002940, WO 2004002944, WO 2004002977, WO 2004003138, WO 2004041201, WO 2004065367, WO 2004096210, WO 2005021568, WO 2005103045, WO 2005123087, WO 2006012078, WO 2006020082, WO 2006065335, WO 2006065590, WO 2006093801, WO 200702602, WO 2007039142, WO 2007039145, WO 2007076034, WO 2007088148, WO 2007092000, and WO2007095269); HCV NS4 Inhibitors (see WO 2005067900 and WO 2007070556); HCV NS5a Inhibitors (see US 2006276511, WO 2006035061, WO 2006100310, WO 2006120251, and WO 2006120252); Toll-like receptor agonists (see WO 2007093901); and other inhibitors (see WO 2000006529, WO 2003101993, WO 2004009020, WO 2004014313, WO 2004014852, and WO 2004035571); PSI-6130 (shown below and disclosed in U.S. Pat. No. 7,429,572); RG7128 (disclosed in U.S. Pat. No. 7,754,699); Compound A (disclosed in US 2010/0081628, see also compound 19a and 19b disclosed in the same application, which are individual diastereomers of compound A); PSI-7977 and PSI-7976 (disclosed in US 2010/0016251 and US 2010/0298257 (Ser. No. 12/783,680) (PSI-7977 (Sp-4) and PSI-7976 (Rp-4)); PSI-353661 (disclosed in WO 2010/075554, see compound 11); telaprevir (also known as VX-950, which is disclosed in US 2010/0015090); boceprevir (disclosed in US 2006/0276405); BMS-790052 (disclosed in WO 2008/021927); ITMN-191 (disclosed in US 2009/0269305 at Example 62-1); ANA-598 (shown below and identified as compound 31 in F. Ruebasam et al. Biorg. Med. Chem. Lett. (2008) 18: 3616-3621; TMC435 (formerly known as TMC435350), INX-189 described in WO 2010/081082.

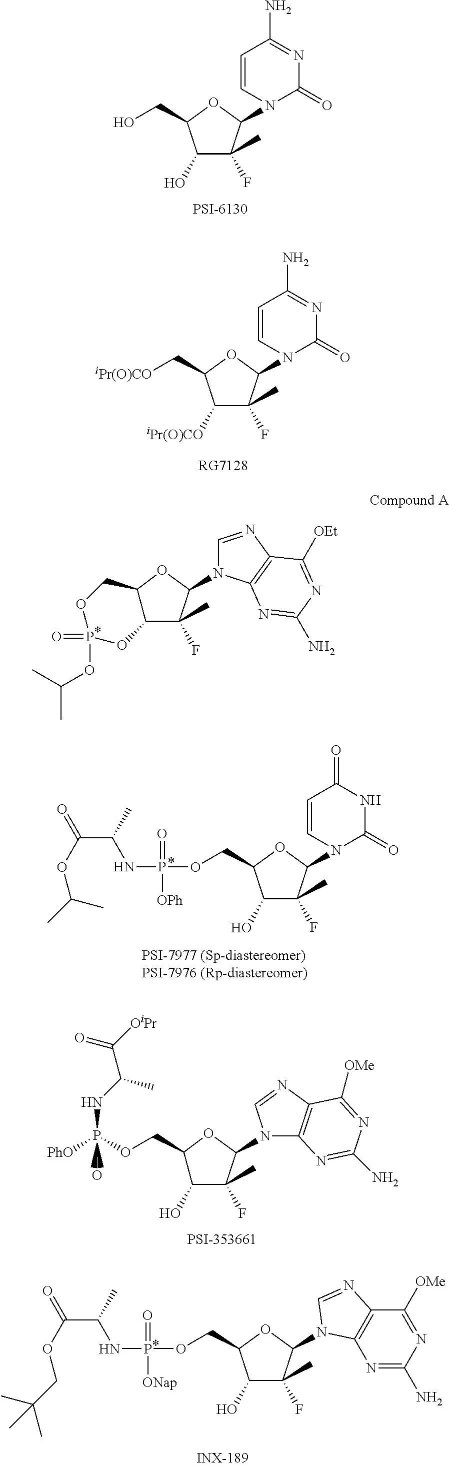

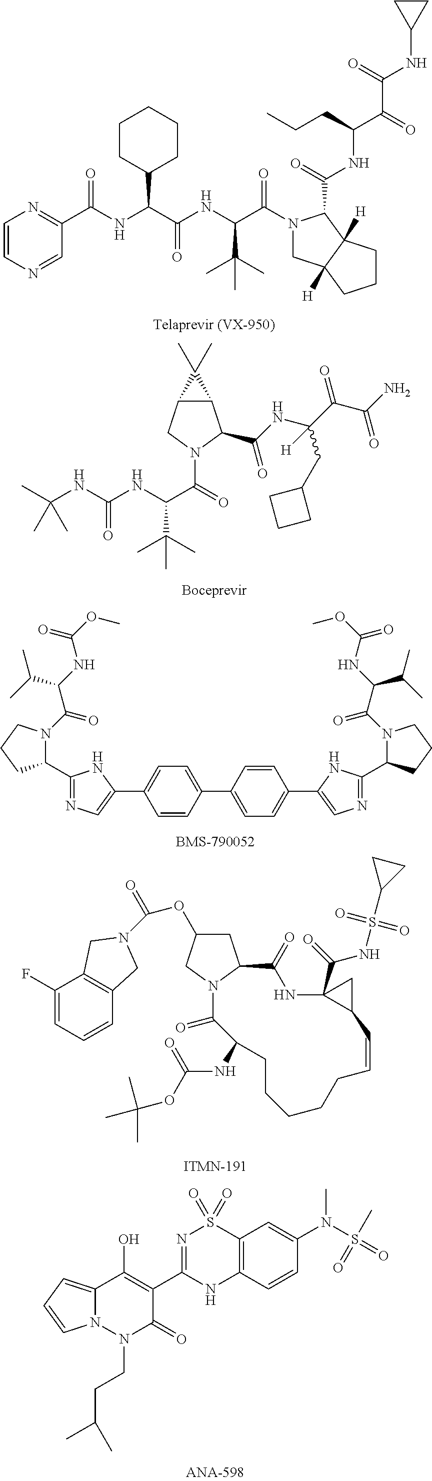

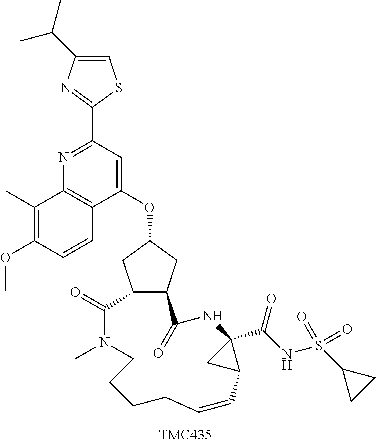

The antiviral agents can be formulated in a manner known to one of ordinary skill. The respective patent documents provide guidance for the respective formulations. The preferred dosage forms of the antiviral agents are those that are approved by the FDA. However, not to be limited, contemplated dosage forms of the antiviral agents are contemplated as follows: RG7128 (500 mg, 1000 mg, or 1500 mg); Compound A (5 mg to 1000 mg and values inbetween); PSI-7977 (100 mg, 200 mg, or 400 mg); A dosage form for VX-950 is disclosed in McHutchison et al. N. Engl. J. Med. (2009) 360(18): 1827-1838; see also WO 2009/038663; Boceprevir (WO 2009/038663).

Additional examples of “another antiviral agent” and contemplated dosages are identified in the following table.

| | | | Clinical | |
|---|---|---|---|---|
| Drug Name | Drug Category | Company | Phase | Dosage |
| RG7128 | Polymerase | Roche in | Phase I | 500 mg |
| | Inhibitor | collaboration with | | BID, 100 mg |
| | | Pharmasset | | BID |
| RG7227 | Protease | Roche in | Phase I | 100 mg TID, |
| | Inhibitor | collaboration with | | 200 mg TID |
| | | Pharmasset | | |
| Telaprevir | Protease | Vertex | Phase II | N/A |
| (VX-950) | Inhibitor | | | |
| VX-222 | Polymerase | Vertex | Phase II | N/A |
| | Inhibitor | | | |
| BMS 790052 | NS5a Inhibitor | Bristol-Myers Squibb | Phase II | 60 mg once a |
| | | | | day or 600 |
| | | | | mg twice a |
| | | | | day |
| BMS 65032 | Protease | Bristol-Myers Squibb | Phase II | 60 mg once a |
| | Inhibitor | | | day or 600 |
| | | | | mg twice a |
| | | | | day |
| BMS-824393 | NS5A Inhibitor | Bristol-Myers Squibb | Phase I | N/A |
| INX-189 | HCV Polymerase | Inhibitex | Phase I | from 3 mg to |
| | Inhibitor | | | 100 mg, once |
| | | | | a day |
| PSI-938 | Polymerase | Pharmasset | Phase I | N/A |
| | Inhibitor | | | |
| PPI-461 | NS5A Inhibitor | Presidio | Phase I | four single |
| | | Pharmaceuticals | | doses |
| | | | | followed by |
| | | | | a 5-day, |
| | | | | once-a-day |
| | | | | dose |
| IDX375 | Polymerase | Idenix | Phase I | 25 mg once |
| | Inhibitor | | | daily (QD), |
| | | | | 50 mg QD, |
| | | | | 100 mg QD, |
| | | | | 200 mg QD, |
| | | | | or 200 mg |
| | | | | twice a day |
| ABT-072 | Polymerase | Abbott | Phase I | N/A |
| | Inhibitor | | | |
| Clemizole | NS4B Inhibitor | Eiger | Phase I | N/A |
| | | BioPharmaceuticals | | |
| MK-3281 | Polymerase | Merck | Phase I | N/A |
| | Inhibitor | | | |
| PSI-7851 | Polymerase | Pharmasset | Phase I | 50 mg, |
| | Inhibitor | | | 100 mg, |
| | | | | 200 mg, or |
| | | | | 400 mg |
| ABT-450 | Protease | Abbott/Enanta | Phase I | N/A |
| HCV | Inhibitor | | | |
| VX-813 | Protease | Vertex | Phase I | N/A |
| | Inhibitor | | | |
| PHX1766 | Protease | Phenomix | Phase I | 400 mg BID |
| | Inhibitor | | | or 800 mg |
| | | | | BID |
| ABT-333 | Polymerase | Abbott | Phase I | N/A |
| | Inhibitor | | | |
| VX-916 | HCV Polymerase | Vertex | Phase I | N/A |
| | Inhibitor | | | |
| RG7128 | Polymerase | Pharmasset/Genentech | Phase I | 500 or |
| | Inhibitor | | | 100 mg BID |
| VX-500 | HCV Protease | Vertex | Phase I | N/A |
| | Inhibitor | | | |
| Filibuvir | HCV Polymerase | Pfizer | Phase II | 200, 300, or |
| (PF- | Inhibitor | | | 0500 mg |
| 00868554) | | | | BID (twice a |
| | | | | day) |
| ACH-1625 | Protease | Achillion | Phase II | 200 or 600 |
| | Inhibitor | | | mg |
| GS-9256 | Protease | Gilead | Phase II | N/A |
| | Inhibitor | | | |
| BI 201335 | Protease | Boehringer Ingelheim | Phase II | 240 mg |
| | Inhibitor | Pharma | | (once-a-day) |
| | | | | or 240 mg |
| | | | | (twice-a-day) |
| VX-222 | Polymerase | Vertex | Phase II | 250, 500, or |
| | Inhibitor | | | 750 mg |
| | | | | twice-a-day; |
| | | | | 1500 mg |
| | | | | once-a-day |
| RG7227 | Protease | InterMune/Genentech | Phase II | N/A |
| (Danoprevir) | Inhibitor | | | |
| ANA598 | Polymerase | Anadys | Phase II | First day 800 |
| | Inhibitor | Pharmaceuticals | | mg BID, |
| | | | | followed by |
| | | | | 200 or 400 |
| | | | | mg twice |
| | | | | daily |
| Vaniprevir | HCV Protease | Merck | Phase II | 300 or 600 |
| (MK-7009) | Inhibitor | | | mg twice a |
| | | | | day; 300 or |
| | | | | 600 mg |
| | | | | once-a-day |
| A-832 | NS5A Inhibitor | ArrowTherapeutics | Phase II | N/A |
| GS 9190 | Polymerase | Gilead | Phase II | N/A |
| | Inhibitor | | | |
| VX-759 | Polymerase | Vertex | Phase II | 400 mg TID, |
| | Inhibitor | | | 800 mg BID, |
| | | | | or 800 mg |
| | | | | TID |
| SCH900518 | Protease | Schering/Merck | Phase II | N/A |
| (Narlaprevir) | Inhibitor | | | |
| BI 207127 | Polymerase | Boehringer Ingelheim | Phase II | N/A |
| | Inhibitor | Pharma | | |
| PSI-7977 | Polymerase | Pharmasset | Phase IIa | 100, 200, or |
| | Inhibitor | | | 400 mg |
| | | | | once-a-day |
| TMC435 | Protease | Medivir/Tibotec | Phase IIa | N/A |
| | Inhibitor | | | |
| BMS 791325 | Polymerase | Bristol-Myers Squibb | Phase IIa | N/A |
| | Inhibitor | | | |
| BMS 650032 | Protease | Bristol-Myers Squibb | Phase IIa/b | N/A |
| | Inhibitor | | | |
| BMS 790052 | NS5a Inhibitor | Bristol-Myers Squibb | Phase IIb | N/A |
| Boceprevir | Protease | Schering | Phase III | 800 mg three |
| (SCH | Inhibitor | | | times a day |
| 503034) | | | | |
| Telaprevir | Protease | Vertex | Phase III | 750 mg |
| (VX 950) | Inhibitor | | | every 8 |
| | | | | hours; 1125 |
| | | | | mg dose |
| | | | | every 12 |
| | | | | hours; |
| BMS-824393 | Type Unknown | Bristol-Myers Squibb | Phase I | N/A |
| SCY-635 | Cyclophilin | SCYNEXIS | Phase I | up to 900 |
| | Inhibitor | | | mg/day |
| ANA773 | TLR Agonist | Anadys | Phase I | 800, 1200, |
| | | Pharmaceuticals | | 1600, or 200 |
| | | | | mg every |
| | | | | other day |
| CYT107 | Immunomodulator | Cytheris | Phase I | N/A |
| CF102 | A3AR Agonist | CAN-FITE | Phase I | N/A |
| IMO-2125 | TLR9 Agonist | Idera | Phase I | N/A |
| | | Pharmaceuticals | | |
| Bavituximab | Anti-Phospholipid | Peregrine | Phase I | N/A |
| (formerly | Therapy | | | |
| Tarvacin) | | | | |
| NOV-205 | Immunomodulator | Novelos Therapeutics | Phase I | N/A |
| SD-101 | TLR9 Agonist | Dynavax | Phase Ib | N/A |
| Miravirsen | microRNA | Santaris | Phase II | up to 12 |
| Formerly | | Pharma | | mg/kg |
| (SPC3649-LNA- | | | | |
| antimiR ™- | | | | |
| 122) | | | | |
| CTS-1027 | Anti- | Conatus | Phase II | N/A |
| | inflammatory | | | |
| Oglufanide | Immunomodulator | Implicit Bioscience | Phase II | N/A |
| disodium | | | | |
| Alinia | Thiazolides | Romark | Phase II | 500 mg |
| (nitazoxanide) | | | | twice daily |
| SCV-07 | Broad Spectrum | SciClone | Phase II | N/A |
| | Immune | | | |
| | Stimulator | | | |
| MitoQ | Inflammation/ | Antipodean | Phase II | N/A |
| (mitoquinone) | Fibrosis Inhibitor | Pharmaceuticals | | |
| Debio 025 | Cyclophilin | Debio | Phase II | 600 to 1000 |
| | Inhibitor | | | mg/day |
| PF-03491390 | Pancaspase | Pfizer | Phase II | 5 mg to 400 |
| (Formerly | Inhibitor | Pharmaceuticals | | mg daily |
| IDN-6556) | | | | (given 1 to 3 |
| | | | | times a day) |

According to the FDA-approved label dated Oct. 8, 2010, the recommended dose of COPEGUS (ribavirin) tablets depends on body weight and the HCV genotype to be treated, as shown in the following table.

| HCV Genotype | PEGASYS Dose* | COPEGUS Dose | Duration |
|---|---|---|---|
| Genotypes 1, 4 | 180 μg | <75 kg = 1000 mg | 48 weeks |
| | | ≧75 kg = 1200 mg | 48 weeks |
| Genotypes 2, 3 | 180 μg | 800 mg | 24 weeks |

Genotypes 2 and 3 showed no increased response to treatment beyond 24 weeks.
*See PEGASYS Package Insert for further details on PEGASYS dosing and administration.

The COPEGUS label further discloses that the recommended duration of treatment for patients previously untreated with ribavirin and interferon is 24 to 48 weeks. The daily dose of COPEGUS is 800 mg to 1200 mg administered orally in two divided doses. The dose should be individualized to the patient depending on baseline disease characteristics (e.g., genotype), response to therapy, and tolerability of the regimen.

### Preparation

Schemes I and II provides general procedures for preparing the piperazinone derivatives disclosed herein.

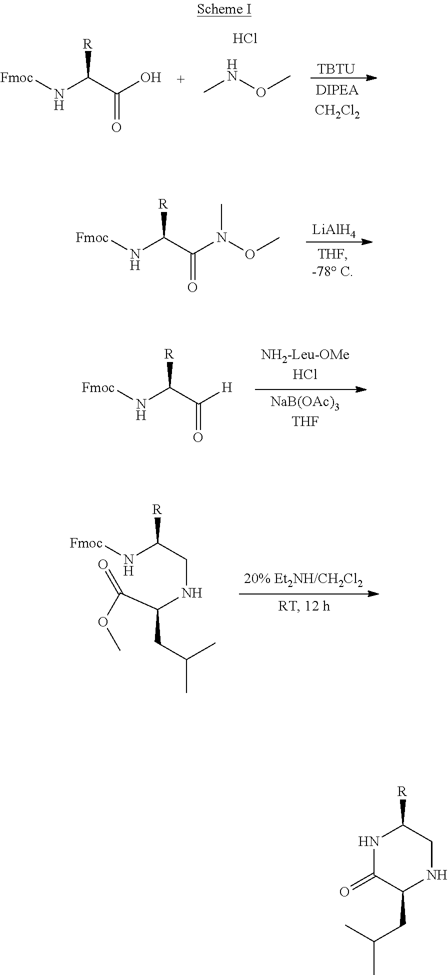

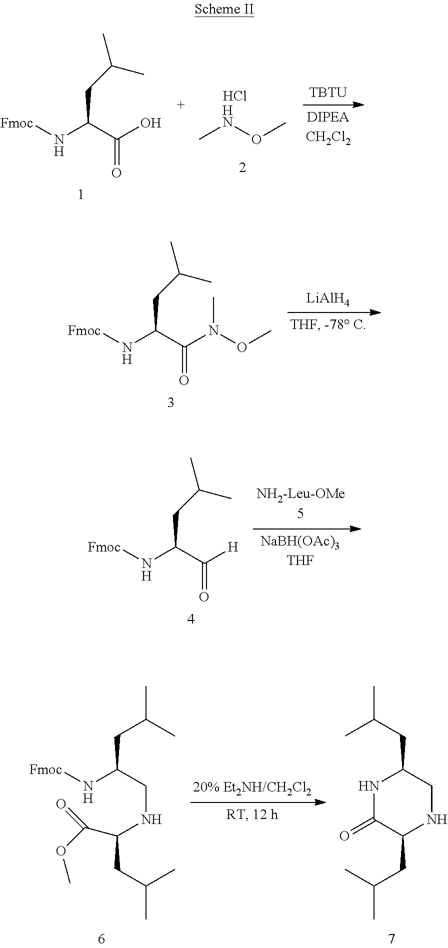

The disclosed reagents are meant to be exemplary only and should not be meant to narrow the scope of the embodiments disclosed below.

A ninth embodiment is directed to a process for preparing a compound disclosed herein by any of the processes disclosed herein.

A tenth embodiment is directed to a process for preparing a compound disclosed herein by any of the processes disclosed herein.

### Preparation

Schemes I and II provides general procedures for preparing the piperazinone derivatives disclosed herein.

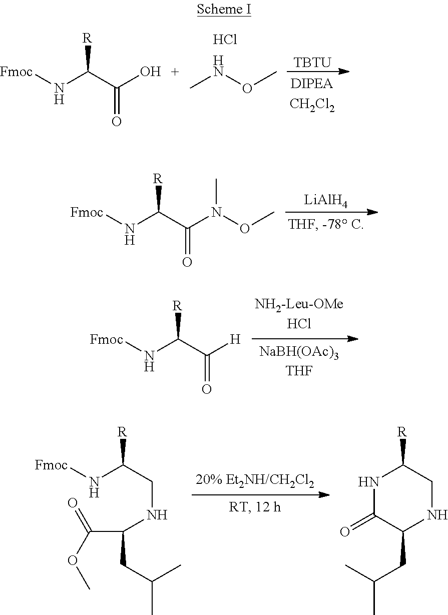

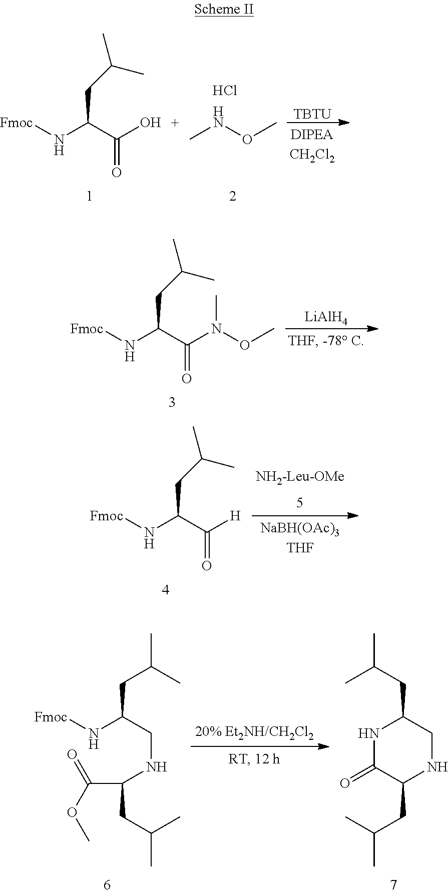

The disclosed reagents are meant to be exemplary only and should not be meant to narrow the scope of the embodiments disclosed below.

A ninth embodiment is directed to a process for preparing a compound disclosed herein by any of the processes disclosed herein.

A tenth embodiment is directed to a process for preparing a compound disclosed herein by any of the processes disclosed herein.

### EXAMPLES

Not to be limited by way of example, the following examples serve to facilitate a better understanding of the disclosure.

### Example 1

### Synthesis of (3S,6S)-3,6-diisobutylpiperazin-2-one(7)

### Step 1: (S)-(9H-fluoren-9-yl)methyl-(1-methoxy(methyl)amino)-4-methyl-1-oxopentan-2-yl)carbamate (3)

A mixture of FMOC-Leu-OH 1 (0.5 g, 1.41 mmol), TBTU (0.68 g, 2.12 mmol), N,O-Dimethylhydroxylamine hydrochloride 2 (0.207 mg, 2.12 mmol) in CH₃CN (5 mL) was stirred at 0° C. for 10 min under nitrogen atmosphere. DIPEA (0.736 mL, 4.23 mmol) was added drop wise (5 min) and stirred for 30 min. The reaction was warmed to RT and stirred for 2.5 h. Solvent was removed by rotavac and the residue was taken up in EtOAc. The mixture was washed sequentially with 1M HCl (15 mL×2), 1M NaHCO₃ (15 mL×2), and brine (20 mL). The solution was dried (Na₂SO₄) and concentrated to dryness to afford compound 3 (580 mg, 99% yield) as a white solid. The crude product 3 was used for next step with out purification.

¹H NMR (400 MHz, CDCl₃): δ 7.76-7.58 (m, 4H), 7.41-7.29 (m, 4H), 5.42 (d, 1H, J=9.2 Hz), 4.82-4.80 (m, 1H), 4.37-4.32 (m, 2H), 4.23-4.20 (t, 1H, J=14.0 Hz), 3.79 (s, 3H), 3.21 (s, 3H), 1.74-1.70 (m, 1H), 1.52-1.48 (m, 1H), 0.99-0.94 (m, 6H).

MS (ESI): m/z 413.2 (M+1)⁺.

### Step 2: (S)-(9H-fluoren-9-yl)methyl (4-methyl-1-oxopentan-2-yl)carbamate (4)

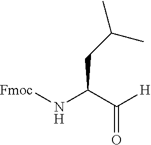

To a solution of substrate 3 (0.4 g, 0.97 mmol) in dry THF (10 mL) at −78° C. was added LiAlH₄ (80 mg, 1.94 mmol) at once and the reaction was stirred for 30 min under Ar atmosphere. The reaction was quenched with 6% HCl in water by adding drop by drop (10 mL, pH 4-5) at −78° C. and the solution was warmed to 0° C. and stirred (during this time ice melted to clear solution and checked the pH) for 5 min. This mixture was partitioned between EtOAc/Brine extracted with EtOAc, the EtOAc layer was combined and washed with brine, and dried (Na₂SO₄) to afford compound 4 (320 mg, 98% yield) as an oil and was used for the next step with out purification.

¹H NMR (400 MHz, CDCl₃): δ 9.58 (s, 1H), 7.77-7.59 (m, 4H), 7.42-7.30 (m, 4H), 5.18 (d, 1H, J=7.2 Hz), 4.45 (d, 2H, J=6.8 Hz), 4.34-4.32 (m, 1H), 4.24-4.21 (t, 1H, J=6.8 Hz), 1.75-1.68 (m, 2H), 1.48-1.39 (m, 1H), 0.98-0.96 (m, 6H).

MS (ESI): m/z 438.1 (M+1)⁺.

### Step 3: (S)-methyl2-(((S)-2-((((9H-fluoren-9-yl)methoxy)carbonyl)amino)-4-methylpentyl)amino)-4-methylpentanoate (6)

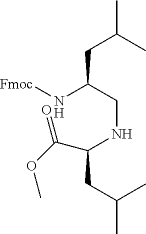

To a well dried mixture of aldehyde 4 (290 mg, 0.86 mmol), HN-Leu-OMe hydrochloride 5 (160 mg, 0.86 mmol) were added dichloroethane (3 mL), and NaBH(OAc)₃ (260 mg, 1.2 mmol). The mixture was stirred for 6 h at RT under nitrogen atmosphere. The reaction was quenched with sat.NaHCO₃ and was extracted with EtOAc and dried over Na2SO4. The solvent was concentrated to dryness and the crude product was dissolved in CH₂Cl₂ and loaded on a column for purification using 0-25% EtOAc/Hex to afford compound 6 (360 mg, 90% yield) as a white solid.

¹H NMR (400 MHz, CDCl₃): δ 7.77-7.59 (m, 4H), 7.41-7.29 (m, 4H), 4.82 (d, 1H, J=5.6 Hz), 4.40 (d, 2H, J=7.2 Hz), 4.24 (t, 1H, J=6.8 Hz), 3.71 (s, 3H), 3.27-3.24 (m, 1H), 2.71-2.42 (m, 2H), 1.76-1.60 (m, 2H), 1.46-1.30 (m, 4H), 0.92-0.87 (m, 12H).

MS (ESI): m/z 467.2 (M+1)⁺.

### Step 4: (3S,6S)-3,6-diisobutylpiperazin-2-one(7)

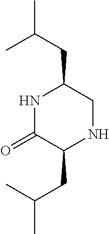

To a solution of substrate 6 (120 mg, 0.26 mmol) in CH₂Cl₂ (1.6 mL) was added diethyl amine (0.4 mL, 3.86 mmol 20% in DCM) at once at RT under nitrogen atmosphere. The reaction was stirred for overnight and the solvent was removed under reduced pressure. The crude product was dissolved in CH₂Cl₂ and loaded on column for purification using 0-25% EtOAc/Hex to afford compound 7 (52 mg, 95% yield) as a white solid.

¹H NMR (400 MHz, CDCl₃): δ 5.83 (s, 1H), 3.47-3.39 (m, 2H), 3.02-2.98 (m, 1H), 2.79-2.74 (m, 1H), 1.80-1.54 (m, 4H), 1.45-1.31 (m, 2H), 0.96-0.90 (m, 12H).

MS (ESI): m/z 213.1 (M+1)⁺.

### Example 2

### (3S,6S)-3-isobutyl-6-isopropylpiperazin-2-one (8)

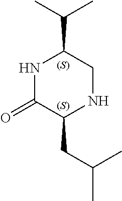

Synthesized from FMOC-L-valine (27.4 g, 0.080 mol) and Hydrochloride salt of L-Leucine methyl ester (3.4 g, 0.023 mol) by the method described for the compound 7 (Scheme II) to afford the product 8 (2.5 g, overall yield 22.17%) as a white solid.

¹H NMR (400 MHz, CDCl₃): δ 5.88 (s, 1H), 3.40 (dd, J=10.4, 4.0 Hz, 1H), 3.09-3.06 (m, 1H), 2.94-2.86 (m, 2H), 1.77-1.72 (m, 2H), 1.59-1.55 (dd, J=10.4, 4.4 Hz, 1H), 1.31-1.23 (m, 1H), 0.95-0.90 (m, 12H).

MS (ESI): m/z 199.0 [M+H]⁺

### Example 3

### (3S,6S)-6-sec-Butyl-3-isobutyl-piperazin-2-one (9)

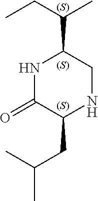

Synthesized from FMOC-Ile-OH (15.0 g, 42.4 mmol) and L-Leucine (8.0 g, 44.5 mmol) by the method described for the compound 7 (Scheme II) to afford the pure product 9 (1.241 g, overall yield: 29.5%) as a white solid.

¹H NMR (400 MHz, CDCl₃): δ 5.64 (br, 1H), 3.41-3.44 (m, 1H), 3.48 (dd, J₁=3.2 Hz, J₂=10.0 Hz, 1H), 3.11-3.15 (m, 1H), 2.68-2.74 (m, 1H), 1.90 (dd, J₁=3.6 Hz, J₂=10.0 Hz, 1H), 1.70-1.80 (m, 1H), 1.60 (br, 1H), 1.41-1.52 (m, 3H), 1.10-1.20 (m, 1H), 0.92-0.97 (m, 12H).

MS (ESI): m/z 213.1 [M+H]⁺

### Example 4

### (3S,6R)-3-Isobutyl-6-methylsulfanylmethyl-piperazin-2-one (10)

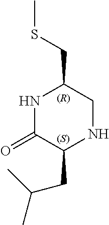

Synthesized from FMOC-L-S-methyl-cys-OH (13.58 g, 0.038 mol) and Hydrochloride salt of L-Leucine methyl ester (5.0 g, 30.7 mmol) by the method described for the compound 7 (Scheme II) to afford the product 10 (1.7 g, overall yield 19.58%) as a white solid.

¹H NMR (400 MHz, DMSO): δ 7.497 (s, 1H), 3.31-3.33 (m, 1H), 3.06-3.10 (m, 1H,), 2.84-2.89 (m, 2H), 2.68-2.72 (m, 1H), 2.53-2.54 (m, 1H), 2.06 (s, 3H) 1.740-1.780 (m, 1H), 1.516-1.541 (m, 1H), 1.369-1.394 (m, 1H), 0.87 (d, J=3.2 Hz, 3H), 0.83 (d, J=3.2 Hz, 3H).

MS (ESI): m/z 217.1 [M+H]⁺

### Example 5

### (3S,6S)-3-Isobutyl-6-phenyl-piperazin-2-one (11)

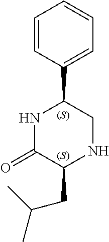

Synthesized from FMOC-L-2-phenylglycine (23.41 g, 0.063 mol) and Hydrochloride salt of L-Leucine methyl ester (8 g, 0.056 mol) by the method described for the compound 7 (Scheme II) to afford the product 11 (2.0 g, overall yield 8.67%) as a white solid.

¹H NMR (400 MHz, DMSO): δ: 7.35-7.40 (m, 2H), 7.24-7.32 (m, 2H), δ 6.12 (s, 1H), 4.62 (q, J=7.2 Hz, 1H), 3.49 (dd, J=10.4, 3.2 Hz, 1H), 3.22-3.27 (m, 1H), 2.98 (dd, J=13.6, 5.2 Hz, 1H), 1.68-1.87 (m, 2H), 1.54-1.61 (m, 1H), 0.94 (d, J=3.2 Hz, 3H), 0.91 (d, J=3.2 Hz, 3H).

MS (ESI): m/z 233.2 [M+H]⁺

### Example 6

### (3S,6S)-6-Cyclopropyl-3-isobutyl-piperazin-2-one (12)

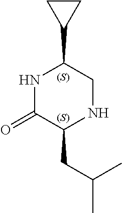

Synthesized from FMOC-L-cyclopropylglycine (13.78 g, 0.043 mol) and Hydrochloride salt of L-Leucine methyl ester (6.0 g, 32.9 mmol) by the method described for the compound 7 (Scheme II) to afford the product 12 (1.7 g, overall yield 20.07%) as a white solid.

¹H NMR (400 MHz, DMSO): δ 7.575 (s, 1H), 3.43 (t, J=4.0 Hz, 1H), 2.81-2.82 (m, 1H), 2.78-2.79 (m, 2H), 2.38-2.39 (m, 2H), 1.72-1.80 (m, 1H), 1.49-1.52 (m, 1H), 1.45-1.47 (m, 1H) 0.95-1.05 (m, 1H), 0.87 (d, J=3.2 Hz, 3H), 0.82 (d, J=3.2 Hz, 3H), 0.42-0.45 m, 1H, 0.32-0.36 (m, 1H), 0.24-0.26 (m, 1H), 0.08-0.12 (m, 1H).

MS (ESI): m/z 197.1 [M+H]⁺

### Example 7

### (3S,6S)-6-Cyclopropylmethyl-3-isobutyl-piperazin-2-one (13)

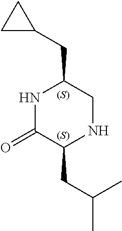

Synthesized from FMOC-cyclopropylalanine (23.6 g, 67.3 mmol) and L-Leucine (6.6 g, 37.0 mmol) by the method described for the compound 7 (Scheme II) to afford the pure product 13 (1.6 g, overall yield: 18.2%) as a white solid.

¹H NMR (400 MHz, DMSO-d₆): δ 7.49 (s, 1H), 3.24-3.27 (m, 1H), 3.05-3.08 (dd, J=4.0, 9.6 Hz, 1H), 2.82-2.86 (dd, J=4.4, 13.2 Hz, 1H), 2.69-2.74 (dd, J=5.2, 12.8 Hz, 1H), 1.72-1.78 (m, 1H), 1.39-1.49 (m, 2H), 1.31-1.36 (m, 2H), 0.81-0.89 (m, 6H) 0.62-0.67 (m, 1H), 0.38-0.40 (m, 2H), 0.01-0.08 (m, 2H).

MS (ESI): m/z 210.9 [M+H]⁺

### Example 8

### (3S,6S)-6-tert-Butyl-3-isobutyl-piperazin-2-one (14)

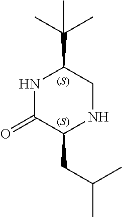

Synthesized from FMOC-L-tert-Leucine (23.0 g, 0.16 mol) and L-Leucine (5.0 g, 27.0 mmol) by the method described for the compound 7 (Scheme II) to afford the pure product 14 (1.0 g, overall yield: 15.8%) as a white solid.

¹H NMR (400 MHz, DMSO-d₆): δ 7.18 (s, 1H), 3.05-3.09 (m, 2H), 2.97-2.99 (m, 1H,), 2.66-2.69 (m, 1H), 2.60-2.64 (m, 1H), 1.76-1.89 (m, 1H), 1.38-1.44 (m, 2H), 0.86-0.89 (m, 15H).

MS (ESI): m/z 213.0 [M+H]⁺

### Example 9

### (3S,6S)-6-(2,2-Dimethyl-propyl)-3-isobutyl-piperazin-2-one (15)

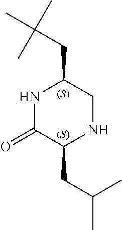

Synthesized from FMOC-gamma-methyl-L-leucine (15.0 g, 0.04 mol) and L-Leucine (3.5 g, 19.35 mmol) by the method described for the compound 7 (Scheme II) to afford the pure product 15 (1.6 g, overall yield: 35.3%) as a white solid.

¹H NMR (400 MHz, DMSO-d₆): δ 7.37 (s, 1H), 3.30 (dd, J=4.0, 5.6 Hz, 1H), 3.07 (dd, J=4, 9.6 Hz, 1H), 2.83 (dd, J=4, 12.8 Hz, 1H), 2.56 (dd, J=6, 13.2 Hz, 1H), 1.90 (s, 1H), 1.74-1.79 (m, 1H), 1.33-1.49 (m, 4H), 0.80-0.90 (m, 15H).

MS (ESI): m/z 227.0 [M+H]⁺

### Example 10

### (3S,6S)-3-Isobutyl-6-propyl-piperazin-2-one (16)

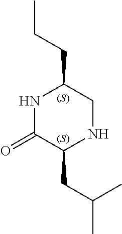

Synthesized from FMOC-L-norvaline (19.82 g, 0.058 mol) and Hydrochloride salt of L-Leucine methyl ester (4.7 g, 26.25 mmol) by the method described for the compound 7 (Scheme II) to afford the pure product 16 (3.4 g, overall 29.09%) as a solid.

¹H NMR (400 MHz, DMSO-d₆): δ: 7.53 (s, 1H), 3.06 (dd, J=4.0, 14 Hz, 1H), 2.78 (dd, J=4, 12.8 Hz, 1H), 2.59 (dd, J=5.2, 12.8 Hz, 1H), 2.49 (dd, J=2, 3.6 Hz, 1H), 2.35 (s, 1H), 1.73-1.80 (m, 1H), 1.36-1.51 (m, 6H), 0.80-0.90 (m, 9H).

MS (ESI): m/z 198.9 [M+H]⁺

### Example 11

### (3S,6S)-6-Cyclopentyl-3-isobutyl-piperazin-2-one (17)

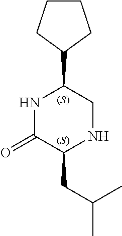

Synthesized from FMOC-L-cyclopentyl-glycine (10.23 g, 0.028 mol) and Hydrochloride salt of L-Leucine methyl ester (6.7 g, 36.8 mmol) by the method described for the compound 7 (Scheme II) to afford the product 17 (1.1 g, overall yield 16.36%) as a solid.

¹H NMR (400 MHz, DMSO-d₆): δ 7.51 (s, 1H), 3.08 (dd, J=3.6, 10 Hz, 1H), 2.93 (dd, J=3.6, 7.6 Hz, 1H), 2.77 (dd, J=4, 12.8 Hz, 1H), 2.68 (dd, J=4.8, 12.8 Hz, 1H), 1.97 (s, 1H), 1.38-1.55 (m, 9H), 1.08-1.22 (m, 3H), 0.80-0.90 (m, 6H).

MS (ESI): m/z 225.1 [M+H]⁺

### Example 12

### (3S,6S)-6-Cyclohexyl-3-isobutyl-piperazin-2-one (18)

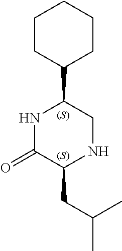

Synthesized from FMOC-L-cyclohexylglycine (22.78 g, 0.06 mol) and Hydrochloride salt of L-Leucine methyl ester (6.0 g, 32.8 mmol) by the method described for the compound 7 (Scheme II) to afford the product 18 (1.2 g, overall yield 7.97%) as a solid.

¹H NMR (400 MHz, DMSO-d₆): δ: 7.45 (s, 1H), 3.08 (dd, J=4.4, 10 Hz, 1H), 2.95 (d, J=5.6 Hz, 1H), 2.76 (dd, J=6, 13.6 Hz, 1H), 2.68 (dd, J=4.4, 13.2 Hz, 1H), 1.59-1.77 (m, 6H), 1.38-1.45 (m, 3H), 1.09-1.16 (m, 3H), 0.89-0.95 (m, 5H), 0.72-0.88 (m, 3H).

MS (ESI) (M+H)⁺ 239.0

### Synthesis of Phenylpropiolic acids

### Example 13

### Synthesis of 3-(4-Fluorophenyl)propiolic acid (19)

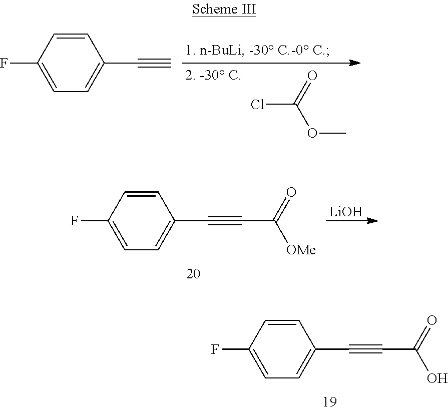

### Example 14

### Synthesis of 3-(4-Fluorophenyl)propiolic acid (19)

### Step 1. Methyl 3-(4-fluorophenyl)propiolate (20)

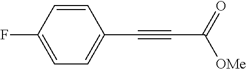

To a solution of 1-ethynyl-4-fluorobenzene (0.29 mL, 2.5 mmol) in anhydrous ether (6.25 mL) was added n-BuLi (1.6M in hexanes, 1.56 mL, 2.5 mmol) drop-wise via syringe at −30° C. After two 30 min stirrings at −30° C. and 0° C. in sequence, the reaction mixture was cooled back to −30° C. before adding methyl carbonochloridate (0.19 mL, 2.5 mmol) in ether (1.25 mL). The mixture was then stirred at −30° C. for 20 min, and gradually warmed to ambient temperature in 1 h. The formed precipitates were filtered off. The filtrate was washed with brine and dried over anhydrous Na₂SO₄. After filtration and concentration, the crude product 20 was used directly for next step without further purification (445 mg, quantitative yield).

¹H NMR (400 MHz, CDCl₃): δ 7.58 (2H, dd, J=5, 9 Hz), 7.08 (2H, dd, J=8.4, 8.8 Hz), 3.84 (3H, s).

### Step 2. 3-(4-Fluorophenyl)propiolic acid (19)

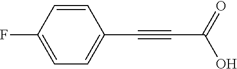

A solution of methyl 3-(4-fluorophenyl)propiolate (20) (445 mg, 2.5 mmol), LiOH (1N aq., 25 mL) in acetone (38 mL) was stirred at rt for 3 hs. The reaction mixture was diluted with EtOAc (100 mL), and acidified by HCl (1N aq.) to pH=4. After separation of the aqueous layer from the organic layer, the aqueous layer was extracted with EtOAc (3×20 mL). The combined organic layers were then dried over Na₂SO₄, filtered, and concentrated in vacuo. The residue was purified by flash silica column to give the product of 3-(4-fluorophenyl)propiolic acid 19 (240 mg, 58% yield) as a white solid.

MS (ESI): m/z 163 [M−1]⁻.

### Example 15

### 3-[4-(trifluoromethyl)-phenyl]-propiolic acid (21)

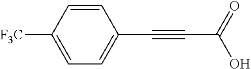

Synthesized from 1-ethynyl-4-(trifluoromethyl)benzene (0.41 mL, 2.5 mmol) by the method described for the compound 19 (Scheme III) to afford the pure product 21 (0.32 g, overall yield: 60%) as a white solid.

¹H NMR (400 MHz, CDCl3): δ 7.68 (2H, d, J=9 Hz), 7.64 (2H, d, J=9 Hz).

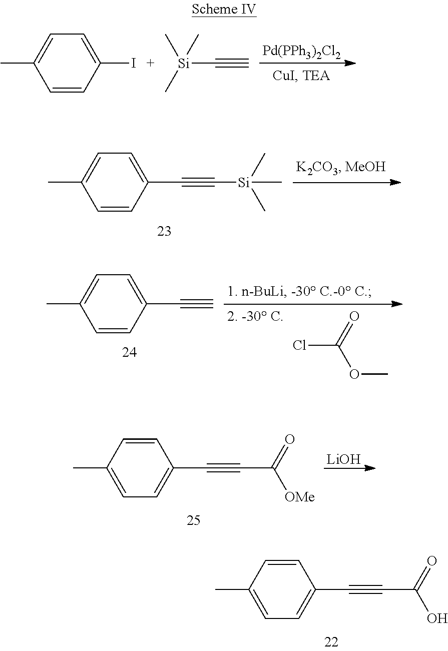

### Example 16

### Synthesis of 3-(p-Tolyl)propiolic acid (21)

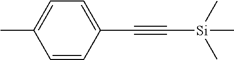

### Step 1. Trimethyl-p-tolylethynyl-silane (23)

To a solution of 1-Iodo-4-methyl-benzene (5 g, 23 mmol), Ethynyl-trimethyl-silane (2.7 g, 27.6 mmol), CuI (2.2 g, 11.5 mmol), PdCl₂(PPh₃)₂ (796 mg, 1.15 mmol) and TEA (10 ml) in DMF was stirred at RT overnight under N₂. The solvent was washed with water and extracted with EtOAc (EA). The organic layer was dried with MgSO₄ and concetracted in vacuo. The crude product was prified by silica gel column chromatography to give product 23 (4 g, 93%) as an oil.

¹H NMR (400 MHz, CDCl3): δ 7.28 (d, J=8 Hz, 2H), 7.06 (d, J=8.4 Hz, 2H), 2.35 (S, 3H), 0.15 (S, 9H).

### Step 2. 1-Ethynyl-4-methyl-benzene (24)

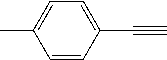

To a solution of 23 (4 g, 21.3 mmol) in MeOH was added K₂CO₃ (5.9 g, 42.6 mmol). The reaction was stirred at RT for 4 h. The solution was filtered. The filtrate was evaporated. The crued product was washed with water and extracted with EA. The organic layer was dried with MgSO₄ and concentrated in vacuo to give product 24 (500 mg, 20.8% yield) as an oil, which was used for the next reaction without further purification.

¹H NMR (400 MHz, CDCl₃): δ 7.28 (d, J=8 Hz, 2H), 7.10 (d, J=8.4 Hz, 2H), 3.06 (S, 1H), 2.35 (S, 3H).

### Step 3. 3-(p-Tolyl)propiolic acid methyl ester (25)

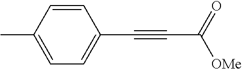

To a solution of 24 (500 mg, 4.3 mmol) in anhydrous THF (20 mL) was added n-BuLi (2.5 M in hexanes, 2 mL, 4.3 mmol) drop-wise via syringe at −30° C. After two 30 min stirrings at −30° C. and 0° C. in sequence, the reaction mixture was cooled back to −30° C. before adding methyl carbonochloridate (460 mg, 4.3 mmol). The mixture was then stirred at −30° C. for 20 min, and gradually warmed to ambient temperature in 1 h. The solvent was evaporated. The crude product was washed with water and extracted with EA. The organic layer was dried with MgSO₄ and concentrated in vacuo to give product 25 (400 mg, 53.5% yield) as a white solid and was used in the following step without further purification.

¹H NMR (400 MHz, CDCl₃): δ 7.36 (d, J=8 Hz, 2H), 7.18 (d, J=8.4 Hz, 2H), 4.03 (S, 3H), 2.34 (S, 3H).

### Step 4. 3-(p-Tolyl)-propiolic acid (22)

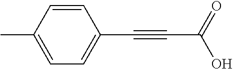

A solution of 25 (400 mg, 2.3 mmol), LiOH.H₂O (276 mg, 6.9 mmol) in methanol:H₂O=1:1 (40 mL) was stirred at ambient temperature for 3 hs. The solvent was evaporated to remove MeOH. The solution was added water and extracted with EA. Then the water layer was acidified by HCl to pH=3 and extracted with EA again. The organic layer was dried with MgSO₄ and concentrated in vacuo to give pure product 22 (320 mg, 86.9% yield) as a white solid.

¹H NMR (400 MHz, CDCl₃): δ 10.18 (s, 1H), 7.51 (d, J=8 Hz, 2H), 7.20 (d, J=8.4 Hz, 2H), 2.39 (S, 3H).

### Example 17

### 3-[(2,4-Difluoro)-phenyl]propiolic acid (26)

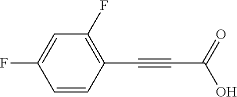

Synthesized from 1-Ethynyl-2,4-difluoro-benzene (1 g, 9.4 mmol) and methyl carbonochloridate (770.4 mg, 7.4 mmol) by the method described for the compound 22 (Scheme IV) to afford the pure product 26 (0.4 g, overall yield: 8.3%) as a white solid.

¹H NMR (400 MHz, CDCl₃): δ 10.18 (s, 1H), 7.43 (dd, J=4.8 Hz, J=4.8 Hz, 1H), 6.75-6.79 (m, 6.75-6.79, 2H).

### Example 18

### 3-[4-(Chloro)-2-(fluoro)-phenyl]propiolic acid (27)

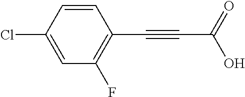

Synthesized from 4-Chloro-2-fluoro-1-iodo-benzene (2.5 g, 10 mmol) and Ethynyl-trimethyl-silane (1.2 g, 12 mmol) and methyl carbonochloridate (348 mg, 3.25 mmol) by the method described for the compound 22 (Scheme IV) to afford the pure product 27 (0.42 g, overall yield: 21.8%) as a white solid.

¹H NMR (400 MHz, CDCl₃): δ 10.28 (s, 1H), 7.40 (dd, J=4.8 Hz, J=4.8 Hz, 1H), 6.70-6.75 (m, 2H).

### Example 19

### 3-[4-(Chloro)-phenyl]propiolic acid (28)

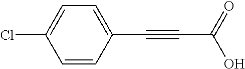

Synthesized from (4-Chloro-phenyl)-propynoic acid methyl ester (500 mg, 2.58 mmol) by the method described for the compound 22 (Scheme IV) to afford the pure product 28 (0.4 g, overall yield: 86.2%) as a white solid.

¹H NMR (400 MHz, CDCl₃): δ 12.36 (s, 1H), 7.79 (d, J=1.6 Hz, 1H), 7.77 (d, J=1.6 Hz, 1H), 6.64 (d, J=1.6 Hz, 1H), 6.62 (d, J=1.6 Hz, 1H).

### Example 20

### 3-[(3,4-Difluoro)-phenyl]-propiolic acid (29)

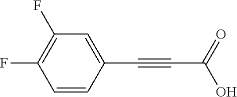

Synthesized from 4-Ethynyl-1,2-difluoro-benzene (1 g, 7.2 mmol) and methyl chloroformate (068 g, 7.2 mmol) by the method described for the compound 22 (Scheme IV) to afford the pure product 29 (0.3 g, overall yield: 22.12%) as a white solid.

¹H NMR (400 MHz, CDCl₃): 7.13-7.37 (m, 3H)

### Example 21

### 3-[4-(Methoxy)-phenyl]-propiolic acid (30)

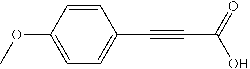

Synthesized from 1-Iodo-4-methoxy-benzene (2 g, 9 mmol) and Ethynyl-trimethyl-silane (1.98, 20 mmol) by the method described for the compound 22 (Scheme IV) to afford the pure product 30 (400 mg, overall yield: 25.2%) as a white solid.

¹H NMR (400 MHz, CDCl₃): δ 7.45-7.49 (m, 4H), 3.82 (s, 3H)

### Example 22

### 3-[3,4,5-(Trifluoro)-phenyl]propiolic acid (31)

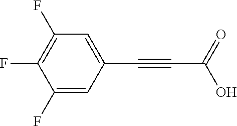

Synthesized from 1,2,3-Trifluoro-5-iodo-benzene (1 g, 3.8 mmol) and Propynoic acid methyl ester (1.25 g, 1.48 mmol) by the method described for the compound 22 (Scheme IV) to afford the pure product 31 (100 mg, overall yield 14.56%) as a yellow solid.

¹H NMR (400 MHz, CDCl₃): δ 7.14-7.24 (m, 2H).

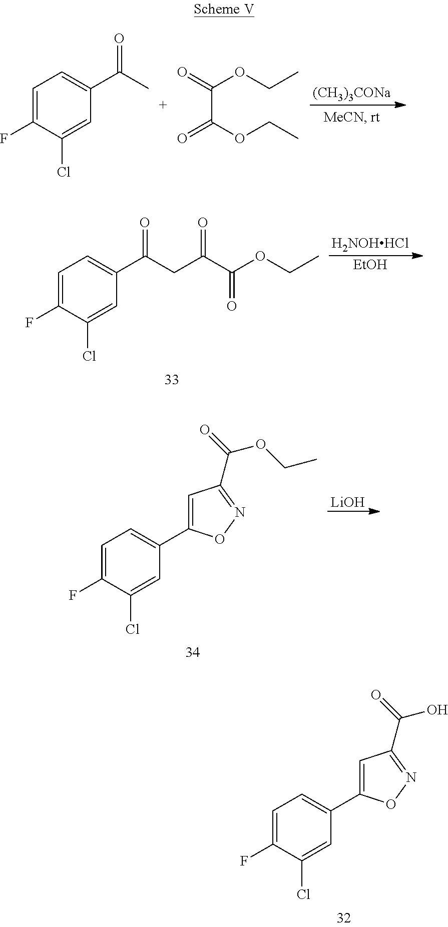

### Example 23

### 5-(3-Chloro-4-fluoro-phenyl)-isoxazole-3-carboxylic acid (32)

### Step 1: 4-(3-Chloro-4-fluoro-phenyl)-2,4-dioxo-butyric acid ethyl ester (33)

To a solution of 1-(3-Chloro-4-fluoro-phenyl)-ethanone (18.8 g, 109 mmol), diethyl oxalate (20.75 g, 142 mmol) in MeCN (100 mL) was added (CH₃)₃CONa (20.98 g, 219 mmol) at 0° C. The resulting mixture was stirred at room temperature overnight. Removed the solvent, the residue was extracted with EA/H₂O. The water phase was adjusted the pH at 4-5 with 4N HCl. Then extracted with EA, the organic layer was dried over Na₂SO₄, concentrated in vacuo to give crude product, and purified by silica column (P:E=20:1) to afford 4-(3-Chloro-4-fluoro-phenyl)-2,4-dioxo-butyric acid ethyl ester (33) as a light yellow solid (10.6 g, 35.8% yield) and used for the next step without further purification.

### Step 2: 5-(3-Chloro-4-fluoro-phenyl)-isoxazole-3-carboxylic acid ethyl ester (34)

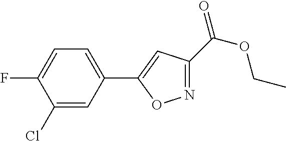

A mixture of 33 (3 g, 11.03 mmol) and NH₂—OH.HCl (2.28 g, 33.09 mmol) in EtOH (50 mL) was heated to reflux overnight. The EtOH was then evaporated and the residue was extracted with EA, dried over Na₂SO₄. After filtration and concentration in vacuo, the crude residue was purified by column to afford 34 as a white solid (2.4 g, 80.8% yield).

¹H NMR (400 MHz, CDCl₃): δ 7.88 (dd, J=6.8, 2.8 Hz, 1H), 7.64-7.68 (m, 1H), 7.26-7.30 (m, 1H), 6.92 (s, 1H), 4.48 (dd, J=14.0, 7.2 Hz, 2H), 1.45 (t, J=7.2 Hz, 3H)

### Step 3: 5-(3-Chloro-4-fluoro-phenyl)-isoxazole-3-carboxylic acid (32)

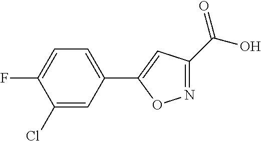

A mixture of 34 (2.2 g, 8.18 mmol), LiOH.H₂O (0.52 g, 12.27 mmol) in methanol (20 mL), THF (20 mL), and H₂O (20 mL) was stirred at room temperature for 2 hrs. Removed the solvent, the residue was extracted with EA/H₂O. The water phase was adjusted the pH at 4-5 with 4N HCl. Then extracted with EA, the organic layer was dried over Na₂SO₄, concentrated in vacuo to afford 32 (1.29 g, 65.5% yield) as a white solid.

¹H NMR (400 MHz, DMSO-d6): δ 14.4 (s, 1H), 8.22 (dd, J=2, 6.8 Hz, 1H), 7.94-7.98 (m, 1H), 7.79 (d, J=2 Hz, 1H), 7.59 (dd, J=8.8, 17.6 Hz, 1H).

MS (ESI): (M+H)⁺ 241.6

### Example 24

### 5-(3,4-Difluorophenyl)isoxazole-3-carboxylic acid (35)

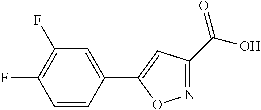

Synthesized from 1-(3,4-difluoro-phenyl)-ethanone (18.8 g, 109 mmol) by the method described for the compound 32 (Scheme V) to afford the product 35 (1.30 g, overall yield 18.9%) as a white solid.

¹H NMR (400 MHz, CDCl₃): δ 7.66-7.61 (1H, m), 7.58-7.54 (1H, m), 7.33-7.27 (1H, m).

### Example 25

### 5-(4-Chloro-2-fluoro-phenyl)-isoxazole-3-carboxylic acid (36)

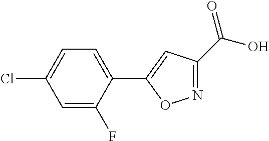

Synthesized from 1-(4-Chloro-2-fluoro-phenyl)-ethanone (13.3 g, 77.4 mmol) by the method described for the compound 32 (Scheme V) to afford the product 36 (1.19530 g, overall yield 19.1%) as a white solid.

¹H NMR (400 MHz, DMSO-d6): δ 14.18 (s, 1H), 7.97-8.02 (m, 1H), 7.71-7.74 (dd, J₁=2.0 Hz, J₂=10.8 Hz, 1H), 7.47-7.50 (m, 1H), 7.14-7.15 (d, J=3.2 Hz, 1H).

MS (ESI): m/z 242.1 [M−H]⁺

### Example 26

### 5-(3-Fluoro-phenyl)-isoxazole-3-carboxylic acid (37)

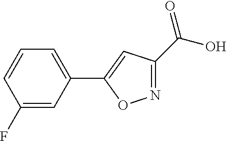

Synthesized from 1-(3-Fluoro-phenyl)-ethanone (10 g, 72.4 mmol) by the method described for the compound 32 (Scheme V) to afford the product 37 (3 g, overall yield 27.8%) as a white solid.

¹H NMR (400 MHz, DMSO-d6): δ 14.14 (s, 1H), 7.79-7.85 (m, 2H), 7.58-7.64 (m, 1H), 7.42 (s, 1H), 7.37-7.39 (m, 1H)

MS (ESI): m/z 208.04 [M+H]⁺.

### Example 27

### 5-(3-Chloro-phenyl)-isoxazole-3-carboxylic acid (38)

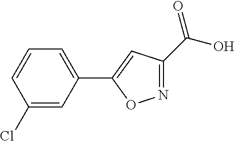

Synthesized from 1-(3-Chloro-phenyl)-ethanone (10 g, 64.9 mmol) by the method described for the compound 32 (Scheme V) to afford the product 38 (1.8 g, overall yield 17.7%) as a white solid.

¹H NMR (400 MHz, DMSO-d6): δ 14.13 (s, 1H), 8.04 (s, 1H), 7.86-7.93 (m, 1H), 7.56-7.62 (m, 2H), 7.52 (s, 1H)

MS (ESI): m/z 224.01 [M+H]⁺.

### Example 28

### 5-(3-Methoxy-phenyl)-isoxazole-3-carboxylic acid (39)

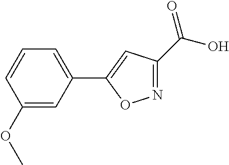

Synthesized from 1-(3-Methoxy-phenyl)-ethanone (12 g, 80 mmol) by the method described for the compound 32 (Scheme V) to afford the product 39 (1.8 g, overall yield 17.7%) as a white solid.

¹H NMR (400 MHz, DMSO-d₆): δ 7.20-7.25 (m, 4H), 6.78-6.84 (m, 1H), 3.58 (s, 3H)

MS (ESI): m/z 219.7 [M+H]⁺.

### Example 29

### 5-(2-Methoxy-phenyl)-isoxazole-3-carboxylic acid (40)

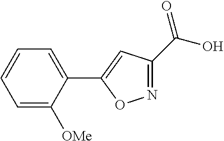

Synthesized from 1-(2-Methoxy-phenyl)-ethanone (10 g, 66.6 mmol) by the method described for the compound 32 (Scheme V) to afford the product 40 (1.4 g, overall yield 12.7%) as a white solid.

¹H NMR (400 MHz, DMSO-d6): δ 14.01 (s, 1H), 7.88 (d, J=7.6 Hz, 1H), 7.51 (t, J=8.0 Hz, 1H), 7.21 (d, J=7.6 Hz, 1H), 7.06-7.12 (m, 2H), 3.45 (s, 3H)

MS (ESI): m/z 219.9 [M+H]⁺.

### Example 30

### 5-(4-tert-Butyl-phenyl)-isoxazole-3-carboxylic acid (41)

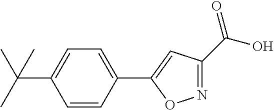

Synthesized from 1-(4-tert-Butyl-phenyl)-ethanone (4.8 g, 27 mmol) by the method described for the compound 32 (Scheme V) to afford the product 41 (0.8 g, overall yield 15.7%) as a white solid.

¹H NMR (400 MHz, DMSO-d6): δ 13.98 (s, 1H), 7.86 (d, J=8.4 Hz, 2H), 7.55 (t, J=9.2 Hz, 2H), 7.35 (s, 1H), 1.31 (s, 9H)

MS (ESI): m/z 245.9 [M+H]⁺.

### Example 31

### 5-(2-Fluoro-phenyl)-isoxazole-3-carboxylic acid (42)

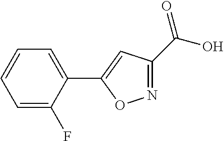

Synthesized from 1-(2-Fluoro-phenyl)-ethanone (18.7 g, 135.46 mmol) by the method described for the compound 32 (Scheme V) to afford the product 42 (2.06 g, overall yield 23.5%) as a white solid.

¹H NMR (400 MHz, DMSO-d₆): δ 14.21 (s, 1H), 7.96-8.00 (m, 1H), 7.60-7.62 (m, 1H), 7.38-7.48 (m, 2H), 7.12-7.13 (m, 1H)

MS (ESI): m/z 207.09 [M+H]⁺.

### Example 32

### 5-(4-Dimethylamino-phenyl)-isoxazole-3-carboxylic acid (43)

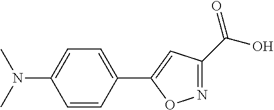

Synthesized from 1-(4-Dimethylamino-phenyl)-ethanone (9.5 g, 58.2 mmol) by the method described for the compound 32 (Scheme V) to afford the product 43 (1.65 g, overall yield 14.6%) as a white solid.

¹H NMR (400 MHz, DMSO-d6): δ 13.82 (s, 1H), 7.71 (d, J=8.8 Hz, 2H), 7.06 (s, 1H), 6.78 (d, J=9.2 Hz, 2H), 2.98 (s, 6H)

MS (ESI): m/z 232.9 [M+H]⁺

### Example 33

### 5-(4-Trifluoromethyl-phenyl)-isoxazole-3-carboxylic acid (44)

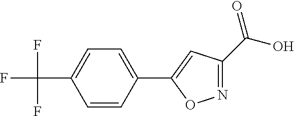

Synthesized from 1-(4-Trifluoromethyl-phenyl)-ethanone (10.1 g, 69 mmol) by the method described for the compound 32 (Scheme V) to afford the product 44 (3.5 g, overall yield 30.9%) as a white solid.

¹H NMR (400 MHz, DMSO-d6): δ 14.06 (s, 1H), 8.14 (d, J=8.4 Hz, 2H), 7.88 (t, J=4.0 Hz, 2H), 7.59 (s, 1H)

### Example 34

### 5-(3,5-Difluoro-phenyl)-isoxazole-3-carboxylic acid (45)

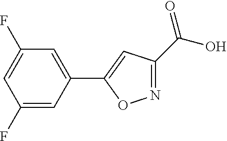

Synthesized from 1-(3,5-Difluoro-phenyl)-ethanone (6.25 g, 40.0 mmol) by the method described for the compound 32 (Scheme V) to afford the product 45 (3.9 g, overall yield 70%) as a white solid.

¹H NMR (400 MHz, DMSO-d6): δ 14.16 (s, 1H), 7.65-7.84 (m, 2H), 7.56 (s, 1H), 7.32-7.44 (m, 1H).

MS (ESI): m/z 226.1 [M−H]⁺

### Example 35

### 5-(4-Methanesulfonyl-phenyl)-isoxazole-3-carboxylic acid (46)

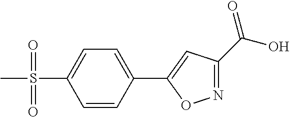

Synthesized from 1-(4-Methanesulfonyl-phenyl)-ethanone (2.0 g, 10 mmol) by the method described for the compound 32 (Scheme V) to afford the product 46 (804.26 mg, overall yield 30%) as a white solid.

¹H NMR (400 MHz, DMSO-d6): δ 8.19-8.22 (m, 2H), 8.06-8.09 (m, 2H), 7.63 (s, 1H), 3.27 (s, 3H),

MS (ESI): m/z 268.1 [M+H]⁺:

### Example 36

### 5-(4-Trifluoromethoxy-phenyl)-isoxazole-3-carboxylic acid (47)

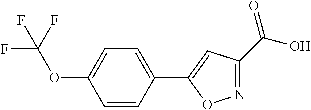

Synthesized from 1-(4-Trifluoromethoxy-phenyl)-ethanone (8.16 g, 40.0 mmol) by the method described for the compound 32 (Scheme V) to afford the product 47 (4.1 g, overall yield 37%) as a white solid.

¹H NMR (400 MHz, DMSO-d6): δ 13.59 (s, 1H), 8.01 (d, J=10.0 Hz, 2H), 7.45-7.47 (d, J=10.0 Hz, 2H), 7.42 (s, 1H).

### Example 37

### 5-(4-Dimethylcarbamoyl-phenyl)-isoxazole-3-carboxylic acid (48)

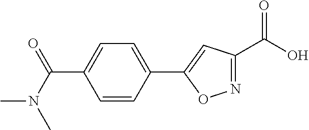

Synthesized from 4-Acetyl-N,N-dimethyl-benzamide (5.85 g, 30 mmol) by the method described for the compound 32 (Scheme V) to afford the product 48 (1.3 g, overall yield 16.4%) as a white solid.

¹H NMR (400 MHz, DMSO-d6): δ 7.99-8.01 (d, J=8.4 Hz, 2H), 7.58-7.56 (d, J=8.4 Hz, 2H), 7.49 (s, 1H), 2.99 (s, 3H), 2.91 (s, 3H)

MS (ESI): m/z 260.8 [M+H]⁺:

### Example 38

### 5-(4-Cyano-phenyl)-isoxazole-3-carboxylic acid (49)

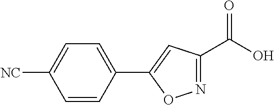

Synthesized from 4-Acetyl-benzonitrile (2.9 g, 20.0 mmol), by the method described for the compound 32 (Scheme V) to afford the product 49 (2.1 g, overall yield 46.8%) as a white solid.

¹H NMR (400 MHz, DMSO-d6): δ 8.14 (d, J=8.4 Hz, 2H), 8.02-8.07 (m, 2H), 7.64 (s, 1H).

### Example 39

### 5-(3-Trifluoromethoxy-phenyl)-isoxazole-3-carboxylic acid (50)

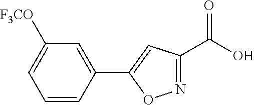

Synthesized from 1-(3-Trifluoromethoxy-phenyl)-ethanone (5.1 g, 29.3 mmol) by the method described for the compound 32 (Scheme V) to afford the product 50 (670.25 mg, overall yield 8.4%) as a white solid.

¹H NMR (400 MHz, DMSO-d6): δ 7.90-7.99 (m, 2H), 7.67-7.71 (m, 1H), 7.58 (s, 1H), 7.52-7.54 (m, 1H).

MS (ESI): m/z 274.1 [M+H]⁺

### Example 40

### 5-(4-chloro-3-fluorophenyl)isoxazole-3-carboxylic acid (51)

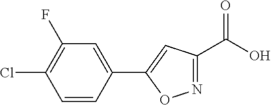

Synthesized from 1-(4-chloro-3-fluorophenyl)ethanone (3.2 g, 18.54 mmol) by the method described for the compound 32 (Scheme V) to afford the product 51 (1.5 g, overall yield 34.6%) as a white solid.

¹H NMR (400 MHz, DMSO-d6): δ 14.18 (s, 1H), δ 7.98-8.00 (m, 1H), δ 7.71-7.78 (m, 2H), 7.30 (s, 1H),

MS (ESI): m/z 242.1 [M−H]⁺

### Example 41

### 5-(5-methylthiophen-2-yl)isoxazole-3-carboxylic acid (52)

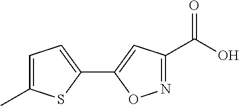

Synthesized from 1-(5-methylthiophen-2-yl)ethanone (860 mg, 6.15 mmol) by the method described for the compound 32 (Scheme V) to afford the product 52 (758.9 mg, overall yield 60%) as a white solid.

¹H NMR (400 MHz, DMSO-d6): δ 7.56-7.57 (d, J=3.6 Hz, 1H), 7.09 (s, 1H), 6.92-6.94 (m, 1H), 2.48 (s, 3H).

MS (ESI): m/z 210.0 [M+H]⁺.

### Example 42

### 5-(3,4,5-trifluorophenyl)isoxazole-3-carboxylic acid (53)

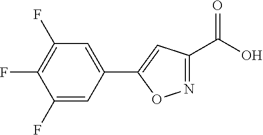

Synthesized from 1-(3,4,5-trifluorophenyl)ethanone (4.5 g, 25.86 mmol) by the method described for the compound 32 (Scheme V) to afford the product 53 (1.65 g, overall yield 30.1%) as a white solid.

¹H NMR (400 MHz, DMSO-d6): δ 14.18 (s, 1H), 7.93-8.01 (m, 2H), 7.54-7.55 (d, J=7.2 Hz, 1H).

### Example 43

### (1R,2R)-2-(phenyl)-cyclopropanecarboxylic acid (54)

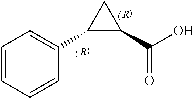

Commercially available 2-(phenyl)-cyclopropanecarboxylic acid trans isomeric mixture (1.62 g) was separated by chiralpak (20 μM, 300×50 mm ID) column using CO₂ and methanol (3:2) mobil phase to furnish product pure trans R,R isomer 54 (650 mg, 40% yield) as a white solid.

¹H NMR (400 MHz, DMSO-d₆): δ 12.34 (s, 1H), 7.30-7.33 (d, J=2.0 Hz, 2H), 7.17-7.21 (d, J=2.0 Hz, 2H), 2.37-2.51 (m, 1H), 1.78-1.83 (m, 1H), 1.41-1.44 (m, 2H).

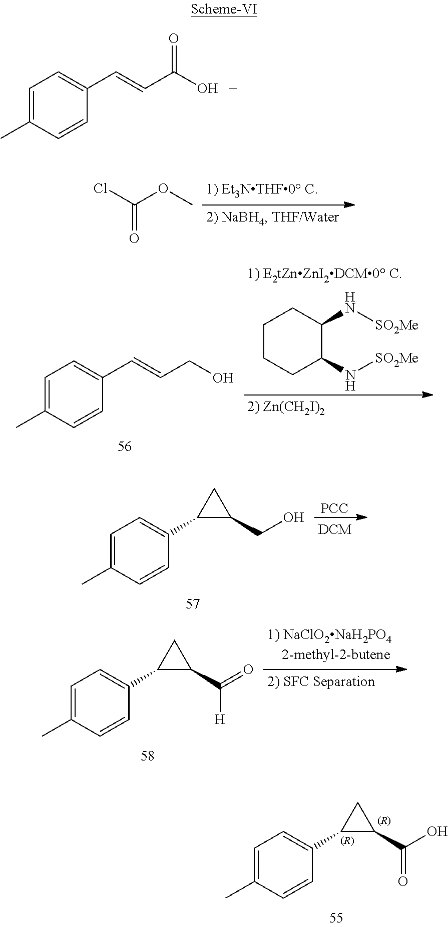

### Example 44

### Synthesis of (1R,2R)-2-(p-Tolyl)cyclopropanecarboxylic acid (55)

### Step 1: 3-p-Tolyl-prop-2-en-1-ol (56)

To a stirred solution of 3-p-Tolyl-acrylic acid (10 g, 61.72 mmol) in anhydrous THF (150 mL) at 0° C. under nitrogen, triethylamine (6.23 mL, 61.72 mmol) was added followed by drop wise addition of methyl chloroformate (5.80 mL, 61.72 mmol) in THF (50 mL). The mixture was stirred at 0° C. for 2 hrs. Reaction mixture was filterd directly into a flask containing sodium borohydride (6.332 g, 166.6 mmol) in water (100 mL) and stirred at room temperature over night. Reaction mixture was treated with aqueous 6N HCl (60 mL) and solvent evaporated. The crude product was dissolved in ethyl acetate (300 mL, washed with brine (200 mL), dried over sodium sulfate and concentrated under reduced pressure. Purification of the crude product using flash chromatography with PE:EA (30:1 to 10:1) furnished product 56 (6 g, 65.7% yield) as a white solid.

¹H NMR (400 MHz, DMSO-d₆) δ 7.30 (d, J=8.4 Hz, 2H), 7.12 (d, J=7.6 Hz, 2H), 6.27-6.52 (m, 2H), 4.85 (t, J=5.6 Hz, 1H), 4.08-4.11 (m, 2H), 2.27-2.32 (m, 3H).

### Step 2: (2-p-Tolyl-cyclopropyl)-methanol (57)

To a dry RB flask flushed with nitrogen, compound 56 (2.56 g, 17.3 mmol), (R,R)—N-(2-Methanesulfonylamino-cyclohexyl)-methanesulfonamide (467 mg, 1.73 mmol). Zinc Iodide (553 mg, 1.73 mmol) and anhydrous DCM (150 mL) was added. The suspension was cooled to 0° C., and diethylzinc (19.03 mL, 19.03 mmol, 1.0M sol. in toluene) was added. After stirring for 30 min at 0° C., the contents were transferred via cannula to a flask that containing a suspension of cyclopropanating reagent in anhydrous DCM. This reagent was prepared in advance by the addition of diethylzinc (18.1 mL, 18.1 mmol, 1.0 M sol in toluene) to a solution of diiodomethane (2.80 mL, 34.6 mmol) in anhydrous DCM (300 mL) at 0° C. with subsequent stirring for 10 min. The combination of the contents of the two flasks led to complete dissolution except for a small amount of zinc iodide. The reaction mixture was stirred at 0° C. for 30 min and then quenched with 1N NaOH (120 mL). The organic layer was removed, and the aqueous layer was extracted with EtOAc (2×250 mL) and the combined organic layers were dried (sodium sulfate) and concentrated in vacuum. The crude product was purified by silica gel flash column with PE:EA (20:1 to 5:1) to furnish oily product 57 (2.3 g, yield: 82.1%) that contains trans R,R enantiomer as major component and trans S,S enantiomer as minor component of the mixture.

¹H NMR (400 MHz, DMSO-d₆): δ 7.03 (d, J=8.0 Hz, 2H), 6.94 (dd, J=6.4, 2 Hz, 2H), 4.61 (t, J=5.6 Hz, 2H), 3.16-3.44 (m, 2H), 2.23 (s, 3H), 1.72 (t, J=4.4 Hz, 1H), 1.15 1.25 (m, 1H), 0.75-0.83 (m, 2H);

### Step 3: 2-p-Tolyl-cyclopropanecarbaldehyde (58)

To a solution of 57 (340 mg, 2.09 mmol) in anhydrous DCM (30 mL), PCC (902 mg, 4.19 mmol) was added and stirred at room temperature for 2 h. The resulting mixture was filtered through celite, and the filtrate evaporated. Crude product was purified by silica gel flash chromatography using eluant PE:EA (30:1 to 20:1) furnished trans isomeric mixture 58 (200 mg, 59.8% yield) as an oily product and used for next reaction without further purification.

¹H NMR (400 MHz, DMSO-d₆): δ 9.07 (d, J=5.2 Hz, 1H), 7.04-7.10 (m, 4H), 2.59-2.64 (m, 2H), 2.25 (s, 1H), 1.64-1.69 (m, 1H), 1.52-1.55 (m, 2H).

### Step 4: (1R,2R)-2-p-Tolyl-cyclopropanecarboxylic acid (55)

To a solution of 58 (1.30 g, 8.125 mmol) in acetone/water (3:1, 80 mL), sodium chlorite (1.897 g, 20.96 mmol), sodium dihydrogen phosphate (1.926 g, 12.35 mmol) and 2-methyl-2-butene (1.89 g, 27.0 mmol) were added and stirred at room temperature for 4 h. Solvents evaporated and the residue was treated with 1N Aq. HCl (150 mL), extracted with ethyl acetate (150 mL), washed with brine, dried (sodium sulfate), and evaporated to give crude product. Crude product was purified by silica gel flash chromatography using eluant petroleum ether and ethyl acetate (10:1 to 3:1) and then followed by SFC seperation furnished product 54 (883.43 mg, 68% yield) as a white solid.

¹H NMR (DMSO-d6) 12.27 (s, 1H), 8.22 (d, J=7.6 Hz, 2H),7.02 (d, J=7.2 Hz, 2H), 2.50 (s, 1H), 2.24 (s, 3H), 1.28-1.73 (m, 3H).

MS (ESI): m/z 175.07 [M+H]⁺.

### Example 45

### (1R,2R)-2-(4-Chloro-phenyl)-cyclopropanecarboxylic acid (59)

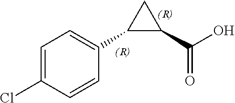

Synthesized from 3-(4-Chloro-phenyl)-acrylic acid (10 g, 54.9 mmol) by the method described for compound 55 (Scheme VI) to furnish product 59 (1.68 g, overall yield 21.9%) as a white solid.

¹H NMR (400 MHz, DMSO-d₆): δ 12.34 (s, 1H), 7.30-7.33 (d, J=2.0 Hz, 2H), 7.17-7.21 (d, J=2.0 Hz, 2H), 2.37-2.51 (m, 1H), 1.78-1.83 (m, 1H), 1.41-1.44 (m, 2H);

MS (ESI): m/z 195.02 [M−H]⁺

### Example 46

### (1R,2R)-2-(4-Fluoro-phenyl)-cyclopropanecarboxylic acid (60)

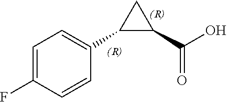

Synthesized from 3-(4-Fluoro-phenyl)-acrylic acid (10 g, 60.20 mmol) by the method described for compound 55 (Scheme VI) to furnish product 60 (699 mg, overall yield 9.71%) as a white solid.

¹H NMR (400 MHz, DMSO-d₆): δ 12.25 (s, 1H), 7.18-7.22 (dd, J₁=5.0 Hz, J₂=8.8 Hz, 2H), 7.06-7.12 (dd, J₁=5.0 Hz, J₂=12.0 Hz, 2H), 2.37-2.50 (m, 1H), 1.75-1.79 (m, 1H), 1.32-1.42 (m, 2H);

MS (ESI): m/z 181.06 [M+H]⁺

### Example 47

### (1R,2R)-2-(3,4-difluorophenyl)cyclopropanecarboxylic acid (61)

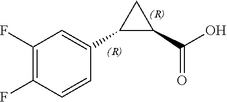

Synthesized from (E)-3-(3,4-difluorophenyl)acrylic acid (10 g, 54.3 mmol) by the method described for compound 55 (Scheme VI) to furnish product 61 (560.13 mg, overall yield 12.0%) as a white solid.

¹H NMR (400 MHz, DMSO-d₆): δ 12.25 (s, 1H), 7.23-7.34 (m, 2H), 7.03-76.0 (m, 1H), 2.38-2.43 (m, 1H), 1.80-1.84 (m, 1H), 1.30-1.42 (m, 2H);

MS (ESI): m/z 197.04 [M−H]⁺

### Example 48

### (1R,2R)-2-(4-methoxyphenyl)cyclopropanecarboxylic acid (62)

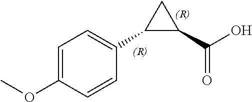

Synthesized from (E)-3-(4-methoxyphenyl)acrylic acid (10 g, 56.5 mmol) by the method described for compound 55 (Scheme VI) to furnish product 62 (465.14 mg, overall yield 12.0%) as a white solid.

¹H NMR (400 MHz, DMSO-d₆) δ 12.25 (s, 1H), 7.08 (dd, J=10.8 Hz, 2.4 Hz, 2H), 6.83 (dd, J=7.2 Hz, 2.0 Hz, 2H), 3.71 (s, 3H), 2.31-2.33 (m, 1H), 1.67-1.71 (m, 1H), 1.25-1.37 (m, 2H);

MS (ESI): m/z 191.07 [M−H]⁺

### Example 49

### (1R,2R)-2-(4-chloro-2-fluorophenyl)cyclopropanecarboxylic acid (63)

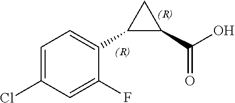

Synthesized from (E)-3-(4-Chloro-2-fluoro-phenyl)-acrylic acid (10 g, 50.00 mmol) by the method described for compound 55 (Scheme VI) to furnish product 63 (748.81 mg, overall yield 12.13%) as a white solid.

¹H NMR (400 MHz, DMSO-d₆): δ 12.51 (s, 1H), 7.39 (d, J=9.6 Hz, 1H), 7.21-7.13 (m, 1H), 2.45-2.41 (m, 1H), 1.86-1.82 (m, 1H), 1.43-1.40 (m, 2H)

### Example 50

### (1R,2R)-2-(2,4-difluorophenyl)cyclopropanecarboxylic acid (64)

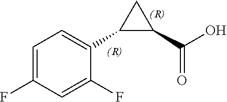

Synthesized from (E)-3-(2,4-Difluoro-phenyl)-acrylic acid (11 g, 60.20 mmol) by the method described for compound 55 (Scheme VI) to furnish product 64 (543.56 mg, overall yield 10.69%) as a white solid.

¹H NMR (400 MHz, DMSO-d₆): δ 7.13-7.22 (m, 2H), 6.97-7.02 (m, 1H), 2.38-2.43 (m, 1H), 1.78-1.82 (m, 1H), 1.38-1.42 (m, 2H).

### Example 51

### (1R,2R)-2-(4-tert-butylphenyl)cyclopropanecarboxylic acid (65)

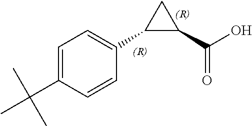

Synthesized from (E)-3-(4-tert-Butyl-phenyl)-acrylic acid (5 g, 24.49 mmol) by the method described for compound 55 (Scheme VI) to furnish product 65 (895.88 mg, overall yield 16.7%) as a white solid.

¹H-NMR (400 MHz, DMSO-d₆): δ 12.30 (s, 1H), 7.28 (dd, J=8.4, 1.6 Hz, 2H), 7.07 (dd, J=8.8, 2.0 Hz, 2H), 2.34-2.32 (m, 1H), 1.76-1.73 (m, 1H), 1.41-1.37 (m, 2H), 1.32 (s, 9H).

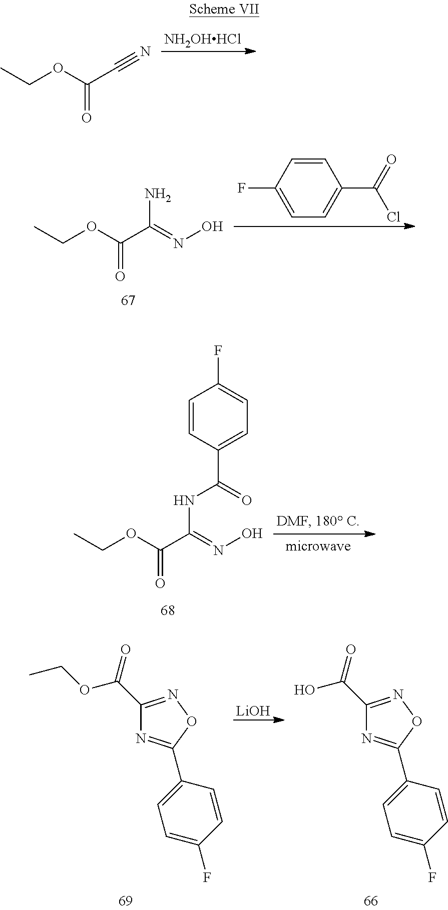

### Example 52

### Synthesis of 5-(4-Fluorophenyl)-1,2,4-oxadiazole-3-carboxylic acid (66)

### Step 1. (Z)-Ethyl 2-amino-2-(hydroxyimino)acetate (67)

Water (12 ml) was added dropwise to a vigorously stirred, room temperature mixture of ethyl cyanoformate (1.98 g, 20 mmol), hydroxylamine hydrochloride (2.08 g, 30 mmol) and sodium carbonate (1.63 g, 15.4 mmol) in ethanol (20 mL) and the resulting solution was stirred until the starting material had been consumed. Solvent was removed in vacuo and the resulting residue was extracted with methylene chloride (3×100 ml.) The combined organic extracts are washed with brine (100 ml), dried (Na2SO4), filtered and concentrated to give the solid product 67 (2.38 g, yield: 90%) and was used for next reaction without further purification.

¹H NMR (400 MHz, CDCl₃): δ 8.85 (1H, s), 5.11 (2H, brs), 4.32 (2H, q, J=7.2 Hz), 1.35 (3H, t, J=7.2 Hz).

### Step 2. (Z)-Ethyl 2-(4-fluorobenzamido)-2-(hydroxyimino)acetate (68)

To a solution of 67 (1 g, 7.57 mmol) in pyridine (2.5 mL) and chloroform (8.4 mL), 4-fluorobenzoyl chloride (0.89 mL, 7.57 mmol) in chloroform (8.4 mL) was added dropwise. After 1 h stirring, the reaction mixture was filtered and the solids were collected as the product of (Z)-ethyl 2-(4-fluorobenzamido)-2-(hydroxyimino)acetate. The collected solids were washed by chloroform and dried in vacuo to give 68 (1.73 g, yield: 90%) and was used for next reaction without further purification.

### Step 3. Ethyl 5-(4-fluorophenyl)-1,2,4-oxadiazole-3-carboxylate (69)

Compound 68 (100 mg, 0.39 mmol) was dissolved in DMF (2.5 mL) and the solution was subjected to microwave irradiation at 180° C. for 30 min. After removal of DMF under vacuum, the residue was purified by column chromatopgraphy to give the product 69 (65 mg, 70% yield) as a white solid.

¹H NMR (400 MHz, CDCl₃): δ 8.25 (2H, dd, J=5.2, 8.8 Hz), 7.27-7.23 (2H, m), 4.55 (2H, q, J=7 Hz), 1.47 (3H, t, J=7 Hz).

### Step 4. 5-(4-Fluorophenyl)-1,2,4-oxadiazole-3-carboxylic acid (66)

To a solution of 69 (1 g, 4.23 mmol) in THF/MeOH/H₂O (1:1:1, 6 mL) was added LiOH (405.6 mg, 16.9 mmol) and stirred at RT overnight. The mixture was concentrated in vacuo and the residue was redissolved in EtOAc (8 mL) and H₂O (2 mL) and acidified with 1N HCl to pH=4. The water layer was separated from the organic layer and extracted by EtOAc (3×10 mL). Organic layers were combined and dried over anhydrous Na₂SO₄. After filtration and concentration in vacuo gave 66 (704 mg, yield: 80%) as a white solid.

¹H NMR (400 MHz, DMSO): δ 8.21 (2H, dd, J=5.2, 8.8 Hz), 7.48 (2H, t, J=8.8 Hz).

### Example 53

### (3S,6S)-3,6-diisobutyl-4-((1R,2R)-2-phenylcyclopropane-carbonyl)piperazin-2-one (70)

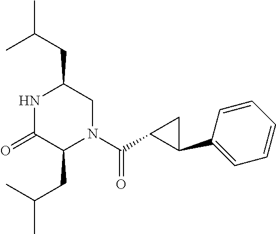

To a mixture of (1R,2R)-2-phenylcyclopropanecarboxylic acid 54 (53.5 mg, 0.33 mmol), TBTU (127.5 mg, 0.396 mmol) and (3S,6S)-3,6-diisobutylpiperazin-2-one 7 (70 mg, 0.33 mmol) were combined and dried under vacuum (2 min). To well dry mixture was added CH₃CN (3 mL) followed by DIPEA (0.17 mL, 0.99 mmol) drop wise (5 min) and stirred for 3 h at RT. After completion of reaction the solvent was concentrated by rotavac and crude residue was taken up in EtOAc. The mixture was washed sequentially with 1M HCl (15 mL×2), 1M NaHCO₃ (15 mL×2), and brine (20 mL once). The solution was dried (Na₂SO₄) and concentrated to dryness. The crude product was purified by flash column chromatography to afford desired product 70 (105.8 mg, 90% yield).

¹H NMR (400 MHz, DMSO-d6): δ 7.50 (s, 1H), 3.23-3.26 (m, 1H), 3.07 (dd, J=9.6, 3.6 Hz, 1H), 2.80 (dd, J=12.8, 4.4 Hz, 1H), 2.58 (dd, J=13.2, 5.2 Hz, 1H), 2.36 (s, 1H), 1.74-1.78 (m, 1H), 1.59-1.65 (m, 1H), 1.28-1.48 (m, 4H), 0.80-0.89 (m, 12H).

MS (ESI): m/z 213.1 (M+H)⁺

### Example 54

### (3S,6S)-3,6-Diisobutyl-4-[(E)-(3-phenyl-acryloyl)]-piperazin-2-one (71)

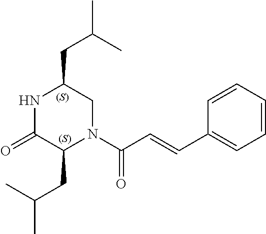

To a mixture of 3,6-diisobutyl-piperazin-2-one (7) substrate (150 mg, 0.71 mmol) and trans cinnamic acid (120 mg, 0.81 mmol) in DCM (10 mL), di-isopropylethylamine (0.7 mL, 4 mmol) and TBTU (250 mg, 0.75 mmol) were added and stirred over night. Solvent removed and the residue disolved in acetone (0.5 mL) and injected into a 40 g silica catridge and performed flash chromatography using Analogix instrument. Elution with ethyl acetate (0 to 100%, 60 min run) in hexanes furnished product 71 (162 mg, 67% yield, recrystallized from 1:4 EtOAC/hexanes) as a white needles.

¹H NMR (400 MHz, CDCl₃): δ 7.74 (d, J=15.2 Hz, 1H), 7.50-7.48 (m, 2H), 7.39-7.37 (m, 3H), 6.83 (dd, 15.2 & 6.4 Hz, 1H), 5.96 (s, 1H), 5.34 (dd, J=9.6 & 3.6 Hz, 1H), 4.79 (dd, J=13.2 & 4 Hz, 1H), 4.58-4.55 (m, 1H), 4.04 (d, J=13.2 Hz), 3.66-3.56 (m, 1H), 3.15 (dd, J=13.2 & 10.8 Hz, 1H), 1.87-1.64 (m, 4H), 1.40-1.34 (m, 2H), 1.06-0.93 (m, 12H).

MS (ESI): m/z 342.6 [M+1]⁺

### Example 55

### (3S,6S)-4-[3-(4-Chloro-phenyl)-acryloyl]-3,6-diisobutyl-piperazin-2-one (72)

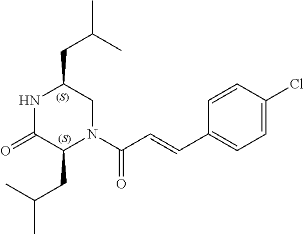

3,6-diisobutyl-piperazin-2-one (7) (60 mg, 0.29 mmol) and p-chlorocinnamic acid (55 mg, 0.3 mmol) were coupled according to the procedure described for compound 71 to furnish 72 (70 mg, 65% yield) as a white solid.

¹H NMR (400 MHz, CDCl₃): δ 8.80-8.60 (m, 2H), 7.85-7.70 (m, 2H), 7.4 (bs, 1H), 6.95 (bs, 1H), 5.95 (s, 1H), 5.38 (bs, 1h), 5.06-4.99 (m, 1H), 4.80 (d, 1H), 4.55 (bs, 1H), 4.05 (m, 1H), 3.10 (m, 1H), 2.70 (m, 1H), 1.90-1.10 (m, 5H), 1.05-0.85 (m, 12H).

MS (ESI): m/z 377.2 [M+1]⁺

### Example 56

### (3S,6S)-3,6-Diisobutyl-4-[3-(4-trifluoromethyl-phenyl)-acryloyl]-piperazin-2-one(73)

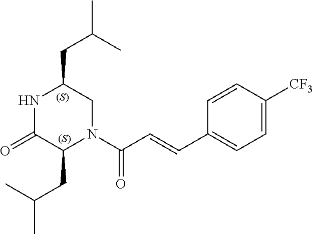

3,6-diisobutyl-piperazin-2-one (7) substrate (60 mg, 0.29 mmol) and p-trifluorocinnamic acid (65 mg, 0.3 mmol) were coupled according to the procedure described for compound 71 to furnish 23 (44 mg, 38% yield) as a white solid.

¹H NMR (400 MHz, CDCl₃): δ 7.77-7.58 (m, 5H), 6.91 (dd, J=7.2 & 8 Hz, 1H), 5.87 (s, 1H), 5.33 (bd, J=9.6 Hz, 1H), 4.81 (bd, J=13.6 Hz, 1H), 4.56-4.53 (m, 1H), 4.00 (bd, J=12.4 Hz, 1H), 3.62 (m, 2H), 2.70 (m, 1H), 1.90-1.69 (m, 2H), 1.44-0.93 (m, 14H).

MS (ESI): m/z 411.2 [M+1]⁺

### Example 57

### (3S,6S)-3-Isobutyl-6-methyl-4-(3-pyridin-3-yl-acryloyl)-piperazin-2-one (74)

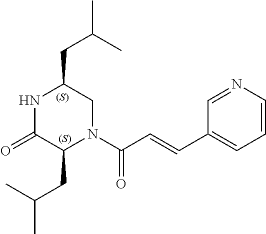

3,6-diisobutyl-piperazin-2-one (7) substrate (60 mg, 0.29 mmol) and 3-pyridylacrylic acid (45 mg, 0.34 mmol) were coupled according to the procedure described for compound 71 to furnish 74 (90 mg, 93% yield) as a colorless syrup.

¹H NMR (400 MHz, CDCl₃): δ 7.70 (dd, J=16 & 4 Hz, 1H), 7.47-7.35 (m, 4H), 6.82 (t, J=14 Hz, 1H), 5.87 (d, J=8 Hz, 1H), 5.33 (bd, J=9.6 Hz, 1H), 4.80 (bd, J=9.6 Hz, 1H), 4.56-4.53 (m, 1H), 4.00 (bd, J=14 Hz, 1H), 3.62 (m, 1H), 2.70 (m, 1H), 1.87-1.69 (m, 2H), 1.50-0.93 (m, 14H).

MS (ESI): m/z 345.2 [M+1]⁺

### Example 58

### (3S,6S)-3,6-Diisobutyl-4-(3-phenyl-but-2-enoyl)-piperazin-2-one (75)

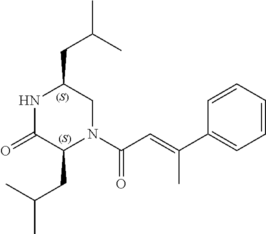

3,6-diisobutyl-piperazin-2-one (7) substrate (60 mg, 0.29 mmol) and beta-methylcinnamic acid (62 mg, 0.3 mmol) were coupled according to the procedure described for compound 71 to furnish 75 (50 mg, 40% yield) as a white solid.

¹H NMR (400 MHz, CDCl₃): δ 7.45-7.34 (m, 5H), 6.27 (s, 1H), 5.87 (s, 1H), 5.29 (m, 1H), 4.76 (bd, J=13.6 Hz, 1H), 4.49 (m, 1H), 3.95 (bd, J=12 Hz, 1H), 3.65-3.50 (m, 2H), 3.09-3.03 (m, 1H), 2.67 (t, J=12 Hz, 1H), 2.29 (s. 3H), 1.82-1.62 (m, 4H), 1.44-1.28 (m, 2H), 1.08-0.91 (m, 12H)

MS (ESI): m/z 356.8 [M+1]⁺

### Example 59

### (3S,6S)-4-(Benzofuran-2-carbonyl)-3,6-diisobutyl-piperazin-2-one (76)

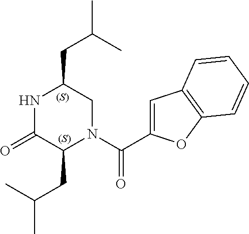

3,6-diisobutyl-piperazin-2-one (7) substrate (60 mg, 0.29 mmol) and benzofuran-2-carboxylic acid (50 mg, 0.3 mmol) were coupled according to the procedure described for compound 71 to furnish 76 (95 mg, 94% yield) as a yellow solid.

¹H NMR (400 MHz, CDCl₃): δ 7.67 (d, J=8 Hz, 1H), 7.49-7.29 (m, 4H), 6.22 (brs, 1H), 5.28 (brs, 1H), 4.69 (m, 1H), 3.78 (m, 1H), 3.16 (m, 1H), 1.80 (m, 4H), 1.38 (m, 2H), 1.06-0.94 (m, 12H).

MS (ESI): m/z 357.5 [M+1]⁺

### Example 60

### (3S,6S)-3,6-Diisobutyl-4-(3-phenyl-allyl)-piperazin-2-one (77)

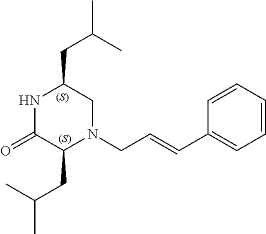

To a mixture of 3,6-diisobutyl-piperazin-2-one (7) substrate (60 mg, 0.29 mmol) and cinnamyl bromide (60 mg, 0.3 mmol) in DCM (5 mL), di-isopropylethylamine (0.35 mL) were added and refluxed at 45° C. over night. Solvent removed and the residue disolved in acetone and injected into a 8 g silica catridge and performed flash chromatography and elution with ethyl acetate (0 to 70%) in hexanes furnished product 77 (90 mg, 97% yield) as a pale yellow solid.

¹H NMR (400 MHz, CDCl₃): δ 7.37-7.22 (m, 5H), 6.54 (d, J=16 Hz, 1H), 6.22-6.15 (m, 1H), 5.73 (s, 1H), 3.71 (m, 1H), 3.41 (d, J=6.4 Hz, 2H), 3.22 (dd, J=9 & 4.5 Hz, 1H), 2.83-2.78 (m, 2H), 1.89 (m, 1H), 1.73-1.52 (m, 4H), 1.39-1.22 (m, 2H), 0.95-0.88 (m, 12H).

MS (ESI): m/z 330.4 [M+1]⁺

### Example 61

### (3S,6S)-3,6-Diisobutyl-4-(2-methyl-3-phenyl-acryloyl)-piperazin-2-one (78)

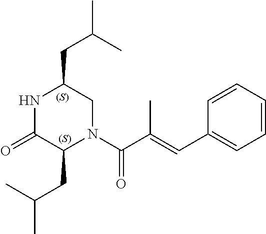

3,6-diisobutyl-piperazin-2-one (7) substrate (60 mg, 0.29 mmol) and alpha-methylcinnamic acid (50 mg, 0.3 mmol)) were coupled according to the procedure described for compound 71 to furnish 78 (95 mg, 95% yield) as a white solid.

¹H NMR (400 MHz, CDCl₃): δ 7.40-7.26 (m, 5H), 6.54 (s, 1H), 5.96 (brs, 1H), 5.21 (s, 1H), 4.66 (brs, 1H), 4.01 (brs, 1H), 3.55 (m, 1H), 3.08 (brs, 1H), 2.80 (s, 3H), 1.81 (m, 1H), 1.80-1.60 (m, 4H), 1.37-1.30 (m, 2H), 1.10-0.92 (m, 12H).

MS (ESI): m/z 357.4 [M+1]⁺

### Example 62

### (3S,6S)-3,6-Diisobutyl-4-(3-p-tolyl-acryloyl)-piperazin-2-one (79)

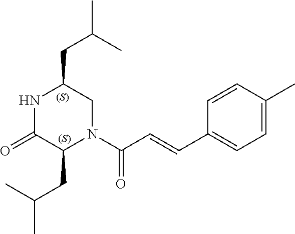

3,6-diisobutyl-piperazin-2-one (7) substrate (60 mg, 0.29 mmol) and 4-methylcinnamic acid (50 mg, 0.3 mmol) were coupled according to the procedure described for compound 71 to furnish 79 (95 mg, 95% yield) as a white solid.

¹H NMR (400 MHz, CDCl₃): δ 7.72 (dd, J=14 & 4.5 Hz, 1H), 7.41 (t, J=6.5 Hz, 1H), 7.19 (d, J=8 Hz, 1H), 6.82-6.76 (dd, J=15 & 7.2 Hz, 1H) (s, 1H), 5.93 (brs, 1H), 5.35 (brd, 1H), 4.81 (dd, J=12.5 & 6.5 Hz, 1H), 457 (brs, 1H), 3.60 (m, 1H), 3.14 (m, 1H), 2.67 (m, 1H), 2.37 (m, 1H), 1.75 (m, 4H), 1.36 (m, 2H), 1.06-0 (m, 12H).

MS (ESI): m/z 357.2 [M+1]⁺

### Example 63

### (3S,6S)-4-[3-(4-Fluoro-phenyl)-acryloyl]-3,6-diisobutyl-piperazin-2-one (80)

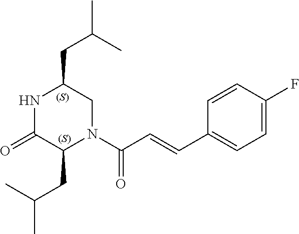

3,6-diisobutyl-piperazin-2-one (7) substrate (53 mg, 0.25 mmol) and para-fluoro-cinnamic acid (50 mg, 0.3 mmol) were coupled according to the procedure described for compound 71 to furnish 80 (80 mg, 94% yield) as a white solid.

¹H NMR (400 MHz, CDCl₃): δ 7.71-7.57 (m, 3H), 7.19-7.11 (m, 3H), 5.19 (dd, J=10.5 & 3.2 Hz, 1H) 4.72 (m, 1H), 4.36 (dd, J=14.4 & 3.2 Hz, 1H), 3.58-3.52 (m, 1H), 3.19 (dd, J=12 & 10.8 Hz, 1H), 2.79-2.74 (m, 1H), 2.37 (m, 1H), 1.85-1.60 (m, 4H), 1.41 (t, J=7.2 Hz, 2H), 1.03-0.94 (m, 12H).

MS (ESI): m/z 361.5 [M+1]⁺

### Example 64

### (3S,6S)-4-[3-(3,4-Dichloro-phenyl)-acryloyl]-3,6-diisobutyl-piperazin-2-one (81)

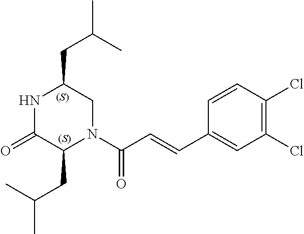

3,6-diisobutyl-piperazin-2-one (7) substrate (53 mg, 0.25 mmol) and 3,4-dichloro-cinnamic acid (50 mg, 0.3 mmol) were coupled according to the procedure described for compound 71 to furnish 81 (80 mg, 94% yield) as a white solid.

¹H NMR (400 MHz, CDCl₃): δ 7.65-7.56 (m, 2H), 7.46 (d, J=8 Hz, 1H), 7.32 (dd, J=8 & 2 Hz, 1H), 6.80 (dd, J=20 & 7.2 Hz, 1H), 5.91 (s, 1H), 5.33 (dd, J=9.6 & 4 Hz, 1H), 5.33 (dd, J=9.6 & 4 Hz, 1H), 4.79 (dd, J=13.6 & 4 Hz, 1H), 4.53 (dd, J=8 & 4 Hz, 1H), 3.65-3.55 (m, 1H), 3.16 (dd, J=14.4 & 11.2 Hz, 1H), 2.69 (dd, J=13.2 & 11.2 Hz, 1H), 1.87-1.65 (m, 4H), 1.40-1.34 (m, 2H), 1.05-0.93 (m, 12H).

MS (ESI): m/z 412.4 [M+1]⁺

### Example 65

### (3S,6S)-4-[3-(3,4-Difluoro-phenyl)-acryloyl]-3,6-diisobutyl-piperazin-2-one (82)

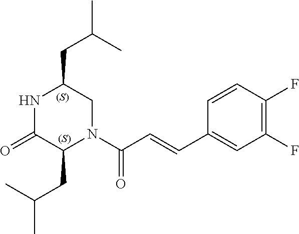

3,6-diisobutyl-piperazin-2-one (7) substrate (53 mg, 0.25 mmol) and 3,4-difluoro-cinnamic acid (60 mg, 0.33 mmol) were coupled according to the procedure described for compound 71 to furnish 82 (88 mg, 98% yield) as a white solid.

¹H NMR (400 MHz, CDCl₃): δ 7.66-7.60 (m, 1H), 7.35-7.13 (m, 3H), 6.73 (dd, J=15.2 & 4.8 Hz, 1H), 6.23 (d, J=15.2, 1H), 5.31 (dd, J=9.6 & 3.6 Hz, 1H), 4.78 (dd, J=13.2 & 4 Hz, 1H), 4.53 (dd, J=8.8 & 4 Hz, 1H), 3.64-3.55 (m, 1H), 3.59 (m, 1H), 3.15 (dd, J=14 & 11.2 Hz, 1H), 2.68 (dd, J=14 & 10.8 Hz, 1H), 1.87-1.65 (m, 4H), 1.38-1.34 (m, 2H), 1.04-0.92 (m, 12H).

MS (ESI): m/z 379.1 [M+1]⁺

### Example 66

### (3S,6S)-4-[3-(3-Fluoro-phenyl)-acryloyl]-3,6-diisobutyl-piperazin-2-one (83)

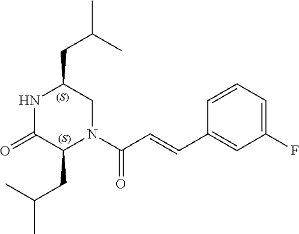

3,6-diisobutyl-piperazin-2-one (7) substrate (53 mg, 0.25 mmol) and 3-fluoro-cinnamic acid (50 mg, 0.30 mmol) were coupled according to the procedure described for compound 71 to furnish 83 (85 mg, 97% yield) as a white solid.

¹H NMR (400 MHz, CDCl₃): δ 7.67 (dd, J=15.2 & 5.6 Hz, 1H), m, 1H), 7.37-7.04 (m, 4H), 6.82 (dd, J=15.2 & 5.6 Hz, 1H), 6.21 (brsd, 1H), 5.32 (dd, J=9.6 & 4 Hz, 1H), 4.79 (dd, J=13.2 & 4 Hz, 1H), 4.56-4.53 (m, 1H), 3.65-3.56 (m, 1H), 3.15 (dd, J=14 & 11.2 Hz, 1H), 2.68 (dd, J=14 & 10.8 Hz, 1H), 1.87-1.67 (m, 4H), 1.38-1.34 (m, 2H), 1.05-0.93 (m, 12H).

MS (ESI): m/z 361.1 [M+1]⁺

### Example 67

### (3S,6S)-3,6-Diisobutyl-4-[3-(3,4,5-trifluoro-phenyl)-acryloyl]-piperazin-2-one (84)

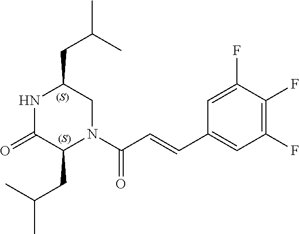

3,6-diisobutyl-piperazin-2-one (7) substrate (60 mg, 0.28 mmol) and 3,4,5-trifluoro-cinnamic acid (70 mg, 0.35 mmol) were coupled according to the procedure described for compound 71 to furnish 84 (110 mg, 96% yield) as a white solid.

¹H NMR (400 MHz, CDCl₃): δ 7.59-7.53 (m, 1H), 7.09-7.03 (m, 2H), 6.73 (brd, J=15.2 Hz, 1H), 6.05 (d, J=15.2, 1H), 5.30 (dd, J=9.6 & 3.6 Hz, 1H), 4.77 (dd, J=13.2 & 4 Hz, 1H), 4.55-4.53 (m, 1H), 3.62-3.56 (m, 1H), 3.59 (m, 1H), 3.07 (dd, J=14 & 11.2 Hz, 1H), 2.64 (dd, J=14 & 10.8 Hz, 1H), 1.95-1.60 (m, 4H), 1.40-1.35 (m, 2H), 1.04-0.92 (m, 12H).

MS (ESI): m/z 397.2 [M+1]⁺

### Example 68

### (3S,6S)-4-[3-(3-Chloro-4-fluoro-phenyl)-acryloyl]-3,6-diisobutyl-piperazin-2-one (85)

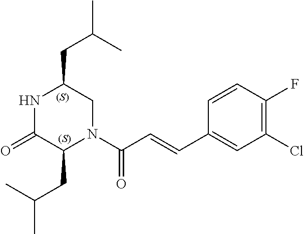

3,6-diisobutyl-piperazin-2-one (7) substrate (110 mg, 0.52 mmol) and trans 4-fluoro-3-chloro-cinnamic acid (110 mg, 0.55 mmol) were coupled according to the procedure described for compound 71 to furnish 85 (100 mg, 48% yield) as a white powder.

¹H NMR (400 MHz, CDCl₃): δ 7.65-7.52 (m, 2H), 7.38-7.34 (m, 1H), 7.15 (t, J=8.8 Hz, 1H), 6.74 (dd, J=15.2 & 6.4 Hz, 1H), 6.26 (d, J=16.8 Hz, 1H), 5.30 (dd, J=9.6 & 3.6 Hz, 1H), 4.76 (dd, J=13.2 & 4 Hz, 1H), 3.99 (brd, J=12.8 Hz, 1H), 3.62-3.55 (m, 1H), 3.15 (dd, J=14 & 11.2 Hz, 1H), 2.67 (dd, J=13.2 & 11.2 Hz, 1H), 1.90-1.60 (m, 4H), 1.39-1.34 (m, 2H), 1.04-0.92 (m, 12H).

MS (ESI): m/z 395.1 [M+1]⁺

### Example 69

### (3S,6S)-4-[3-(4-Chloro-2-fluoro-phenyl)-acryloyl]-3,6-diisobutyl-piperazin-2-one (86)

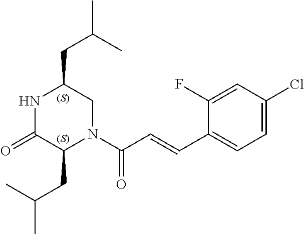

3,6-diisobutyl-piperazin-2-one (7) (200 mg, 0.94 mmol) ande 3-(4-Chloro-2-fluoro-phenyl)-acrylic acid (189 mg, 0.94 mmol) were coupled according to the procedure described for compound 71 to furnish 86 (302 mg, 81.35% yield).

¹H NMR (400 MHz, CDCl₃): δ 7.66-7.73 (m, 1H), 7.38-7.42 (m, 1H), 7.12-7.17 (m, 2H), 6.97 (t, J₁=J₂=14.0 Hz, 1H), 6.30 (d, J=17.2 Hz, 1H), 5.31 (dd, J₁=3.2 Hz, J₂=9.2 Hz, 1H), 4.01 (d, J=11.2 Hz, 1H), 3.57-3.63 (m, 1H), 3.09-3.16 (m, 1H), 1.70-1.86 (m, 4H), 1.34-1.38 (m, 2H), 0.92-1.05 (m, 12H).

MS (ESI): m/z 395.1 [M+1]⁺

### Example 70

### (3S,6S)-4-[3-(2,4-Difluoro-phenyl)-acryloyl]-3,6-diisobutyl-piperazin-2-one (87)

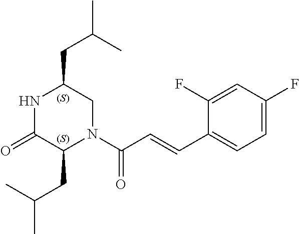

3,6-diisobutyl-piperazin-2-one (7) (200 mg, 0.94 mmol), 3-(2,4-Difluoro-phenyl)-acrylic acid (173 mg, 0.94 mmol) were coupled according to the procedure described for compound 71 to furnish 87 (278 mg, 78.14% yield).

¹H NMR (400 MHz, CDCl₃): δ 7.66-7.73 (m, 1H), 7.41-7.47 (m, 1H), 6.81-6.97 (m, 3H), 6.43 (d, J=13.8 Hz, 1H), 5.29 (dd, J₁=4.0 Hz, J₂=10.0 Hz, 1H), 4.01 (d, J=11.2 Hz, 1H), 3.57-3.60 (m, 1H), 3.08-3.14 (m, 1H), 1.72-1.86 (m, 4H), 1.34-1.38 (m, 2H), 0.91-1.02 (m, 12H).

MS (ESI): m/z 379.1 [M+H]⁺

### Example 71

### 2-[(2S,5S)-(2,5-Diisobutyl-3-oxo-piperazin-1-yl)]-N-(2,6-dimethyl-phenyl)-acetamide (88)

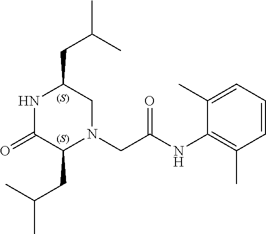

To a solution of 3,6-diisobutyl-piperazin-2-one (7) (150 mg, 0.706 mmol) in DMF (10 mL) was added 2-Chloro-N-(2,6-dimethyl-phenyl)-acetamide (250 mg, 1.26 mmol), K₂CO₃ (150 mg, 1.08 mmol) and NaI (160 mg, 1.06 mmol). The mixture was stirred at 70° C. for 12 hours. The mixture was diluted with water (10 mL), extracted with EtOAc (3×15 mL). The combined organic solution was washed with brine, dried over Na₂SO₄, conc., and purified prep HPLC under formic acid condition to give the product 88 (141.23 mg, 54.01% yield) as a white solid.

¹H NMR (400 MHz, DMSO-d₆): δ 9.12 (s, 1H), 7.62 (s, 1H), 7.01-7.04 (m, 3H), 3.54-3.55 (m, 1H), 3.38-3.42 (m, 2H), 3.06-3.08 (m, 1H), 2.80-2.82 (m, 1H), 2.72-2.74 (m, 1H), 2.09 (s, 6H), 1.86-1.87 (m, 1H), 1.58-1.64 (m, 2H), 1.34-1.50 (m, 2H), 1.18-1.21 (m, 1H), 0.80-0.89 (m, 12H).

MS (ESI): m/z 374.4 [M+H]⁺

### Example 72

### (3S,6S)-3,6-Diisobutyl-4-(4-phenoxybenzoyl)piperazin-2-one (89)

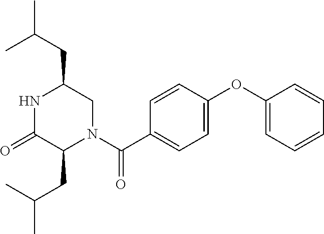

To a solution of 7 (100 mg, 0.47 mmol), HOBT (79.6 mg, 0.589 mmol), 4-phenoxybenzoic acid (120.8 mg, 0.564 mmol), EDC (108.1 mg, 0.564 mmol) in CH₃CN (5 mL) was added DIPEA (0.2 mL, 1.174 mmol) dropwise. The mixture was stirred at rt overnight. After concentrated in vacuo, the residue was purified by column to give the product of (3S,6S)-3,6-Diisobutyl-4-(4-phenoxybenzoyl)piperazin-2-one 89 (66.2 mg, yield: 34.5%) as white solid.

¹H NMR (400 MHz, CD₃OD): δ 7.45-7.38 (4H, m), 7.18 (1H, t, J=7.4 Hz), 7.07-7.03 (4H, m), 5.20-5.08 (1H, m), 3.84-3.78 (1H, m), 3.54-3.44 (1H, m), 3.22-3.10 (1H, m), 1.90-1.66 (3H, m), 1.54-1.20 (4H, m), 1.10-0.94 (6H, m), 0.86-0.66 (5H, m)

MS (ESI): m/z 309.2 [M+1]⁺

### Example 73

### (3S,6S)-4-((E)-3-(Benzo[d][1,3]dioxol-5-yl)acryloyl)-3,6-diisobutylpiperazin-2-one (90)

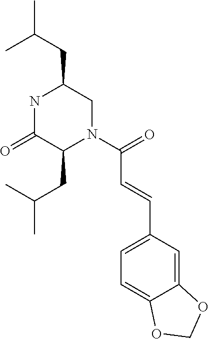

According to the method described for the synthesis of compound 89, 7 (99.8 mg, 0.47 mmol) was coupled with (E)-3-(benzo[d][1,3]dioxol-5-yl)acrylic acid (108.4 mg, 0.564 mmol) to give the product 90 (75.1 mg, yield: 41.3%) as white solids.

¹H NMR (400 MHz, CDCl₃): δ 7.64 (1H, d, J=14.8 Hz), 7.00-6.97 (2H, m), 6.79 (1H, d, J=8.4 Hz), 6.64 (1H, d, J=15.2 Hz), 6.24 (1H, s), 5.99 (2H, s), 4.78 (1H, d, J=13.6 Hz), 4.58-4.50 (1H, m), 3.62-3.52 (1H, m), 2.68-2.62 (1H, m), 1.89-1.61 (4H, m), 1.42-1.32 (2H, m), 1.05-0.88 (12H, m).

MS (ESI): m/z 387.1 [M+1]⁺

### Example 74

### (3S,6S)-3,6-Diisobutyl-4-((E)-3-(4-nitrophenyl)acryloyl)piperazin-2-one(91)

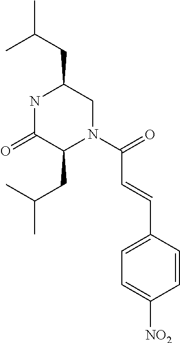

According to the method described for the synthesis of compound 89, 7 (99.8 mg, 0.47 mmol) was coupled with (E)-3-(4-nitrophenyl)acrylic acid (108.9 mg, 0.564 mmol) to give the product 91 (81.8 mg, yield: 44.9%) as a white solid.

¹H NMR (400 MHz, CDCl₃): δ 8.21 (2H, d, J=8.4 Hz), 7.73 (1H, d, J=15.2 Hz), 7.63 (2H, d, J=8.4 Hz), 6.95 (1H, d, J=15.2 Hz), 6.50 (1H, s), 4.77 (1H, d, J=13.2 Hz), 4.55-4.49 (1H, m), 3.64-3.52 (1H, m), 2.72-2.66 (1H, m), 1.91-1.58 (4H, m), 1.42-1.32 (2H, m), 1.04-0.89 (12H, m).

MS (ESI): m/z 388.1 [M+1]⁺.

### Example 75

### (3S,6S)-3,6-Diisobutyl-4-((E)-3-(4-(methylsulfonyl)phenyl)acryloyl)piperazin-2-one (92)

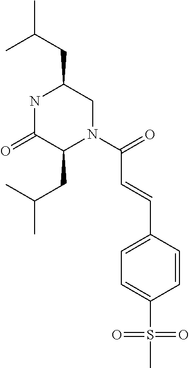

According to the method described for the synthesis of compound 89, 7 (99.8 mg, 0.47 mmol) was couple with (E)-3-(4-(methylsulfonyl)phenyl)acrylic acid (127.6 mg, 0.564 mmol) to give the product 92 (150.5 mg, yield: 76.1%) as a white solid.

¹H NMR (400 MHz, CDCl₃): δ 7.94 (2H, d, J=8.4 Hz), 7.73 (1H, d, J=15.6 Hz), 7.66 (2H, d, J=8.4 Hz), 6.94 (1H, d, J=15.6 Hz), 6.33 (1H, s), 4.78 (1H, d, J=3.8, 13.2 Hz), 4.55-4.51 (1H, m), 3.65-3.52 (1H, m), 3.06 (3H, s), 2.69 (1H, d, J=11.2, 13.2 Hz), 1.91-1.62 (4H, m), 1.43-1.33 (2H, m), 1.06-0.91 (12H, m)

MS (ESI): m/z 421.4 [M+1]⁺

### Example 76

### (3S,6S)-4-((E)-3-(2-Fluoro-4-(trifluoromethyl)phenyl)acryloyl)-3,6-diisobutylpiperazin-2-one (93)

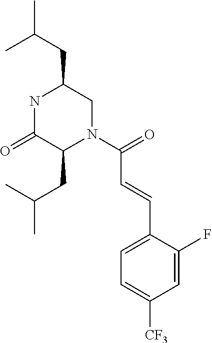

According to the method described for the synthesis of compound 89, 7 (99.8 mg, 0.47 mmol) was coupled with (E)-3-(2-fluoro-4-(trifluoromethyl)phenyl)acrylic acid (132 mg, 0.564 mmol) to give the product 93 (79.2 mg, yield: 39.3%) as a white solid.

¹H NMR (400 MHz, CDCl₃): δ 7.77 (1H, d, J=15.2 Hz), 7.61-7.57 (1H, m), 7.44 (1H, d, J=8 Hz), 7.38 (1H, d, J=8 Hz), 7.07 (1H, d, J=15.2 Hz), 6.24 (1H, s), 4.79 (1H, dd, J=4, 13.4 Hz), 4.54-4.51 (1H, m), 3.66-3.52 (1H, m), 2.70 (1H, dd, J=11.2, 13.4 Hz), 1.92-1.62 (4H, m), 1.43-1.34 (2H, m), 1.08-0.92 (12H, m).

MS (ESI): m/z 429.4 [M+1]⁺.

### Example 77

### (3S,6S)-4-((E)-3-([1,1′-Biphenyl]-4-yl)acryloyl)-3,6-diisobutylpiperazin-2-one (94)

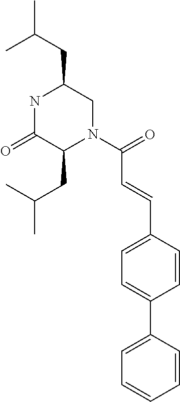

According to the method described for the synthesis of compound 89, 7 (99.8 mg, 0.47 mmol) was coupled with (E)-3-([1,1′-biphenyl]-4-yl)acrylic acid (126.3 mg, 0.564 mmol) to give the product 94 (95.3 mg, yield: 48.4%) as a white solid.

¹H NMR (400 MHz, CDCl₃): δ 7.79 (1H, d, J=15.6 Hz), 7.64-7.57 (6H, m), 7.47-7.43 (2H, m), 7.38-7.35 (1H, m), 6.87 (1H, d, J=15.6 Hz), 6.15 (1H, s), 4.82 (1H, dd, J=3.6, 13.4 Hz), 4.62-4.58 (1H, m), 3.65-3.55 (1H, m), 2.69 (1H, dd, J=10.8, 13.2 Hz), 1.92-1.67 (4H, m), 1.42-1.34 (2H, m), 1.07-0.93 (12H, m).

MS (ESI): m/z 419.2 [M+1]⁺

### Example 78

### (3S,6S)-4-((E)-3-(3,5-Difluorophenyl)acryloyl)-3,6-diisobutylpiperazin-2-one (95)

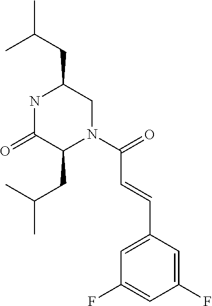

According to the method described for the synthesis of compound 89, 7 (99.8 mg, 0.47 mmol) was coupled with (E)-3-(3,5-difluorophenyl)acrylic acid (103.8 mg, 0.564 mmol) to give the product 95 (58.5 mg, yield: 32.9%) as a colorless oil.

¹H NMR (400 MHz, CDCl₃): δ 7.61 (1H, d, J=15.2 Hz), 7.00 (2H, d, J=6 Hz), 6.83 (2H, d, J=15.2 Hz), 6.25 (1H, s), 5.31-5.29 (1H, m), 4.82-4.74 (1H, m), 3.65-3.52 (1H, m), 3.19-3.12 (1H, m), 1.92-1.64 (4H, m), 1.43-1.33 (2H, m), 1.06-0.92 (12H, m).

MS (ESI): m/z 379.2 [M+1]⁺.

### Example 79

### (3S,6S)-4-((E)-3-(2,4-difluorophenyl)acryloyl)-3-isobutyl-6-propylpiperazin-2-one (96)

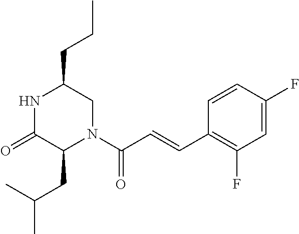

(3S,6S)-3-Isobutyl-6-propyl-piperazin-2-one (17) and (E)-3-(2,4-difluorophenyl)acrylic acid were coupled according to the procedure described for the preparation of 71 to furnish 96 (86 mg, 93% yield) as white solid.

¹H NMR (400 MHz, CDCl₃): δ 7.66-7.59 (m, 1H), 7.36-7.14 (m, 3H), 6.76-6.71 (m, 1H), 6.10 (s, 1H), 5.33-4.77 (m, 1H) 4.54-3.99 (m, 1H), 3.57-3.50 (m, 1H), 3.21-2.67 (m, 1H), 1.88-1.35 (m, 7H), 1.13-0.82 (m, 9H);

MS (ESI): m/z 365.1 (M+1)⁺

### Example 80

### (3S,6S)-3,6-Diisobutyl-4-(5-phenylfuran-2-carbonyl)piperazin-2-one(97)

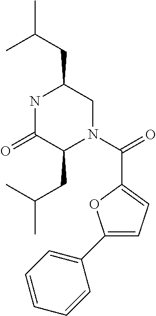

According to the method described for the synthesis of compound 89, 7 (99.8 mg, 0.47 mmol) was coupled with 5-phenylfuran-2-carboxylic acid (106 mg, 0.564 mmol) to give the product 97 (51.5 mg, 28.6% yield) as a white solid.

¹H NMR (400 MHz, CDCl₃): δ 7.67 (2H, d, J=7.6 Hz), 7.44-7.39 (2H, m), 7.36-7.33 (1H, m), 7.21 (1H, d, J=3.6 Hz), 6.75 (1H, d, J=3.6 Hz), 6.04 (1H, brs), 5.35-5.26 (1H, s), 4.77 (1H, dd, J=4, 20 Hz), 3.82-3.73 (1H, m), 3.25-3.12 (1H, m), 1.92-1.65 (4H, m), 1.41-1.37 (2H, m), 1.00-0.89 (12H, m).

MS (ESI): m/z 383.1 [M+1]⁺

### Example 81

### (3S,6S)-3,6-Diisobutyl-4-(2-phenylthiazole-4-carbonyl)piperazin-2-one (98)

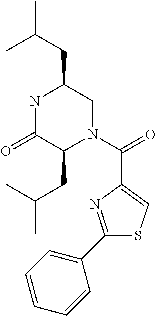

According to the method described for the synthesis of compound 89, 7 (99.8 mg, 0.47 mmol) was coupled with 2-phenylthiazole-4-carboxylic acid (115.8 mg, 0.564 mmol) to give the product 98 (57.8 mg, 30.8% yield) as a colorless solid.

¹H NMR (400 MHz, CDCl₃): δ 8.07 (1H, s), 7.94-7.89 (2H, m), 7.45-7.43 (3H, m), 6.48 (1H, s), 5.31-5.29 (1H, m), 5.13 (1H, d, J=14 Hz), 3.92-3.84 (1H, m), 3.08 (1H, dd, J=10.8, 14 Hz), 1.90-1.66 (4H, m), 1.41-1.24 (2H, m), 1.09-0.82 (12H, m).

MS (ESI): m/z 400.1 [M+1]⁺.

### Example 82

### (3S,6S)-3,6-Diisobutyl-4-(2-phenyloxazole-4-carbonyl)piperazin-2-one (99)

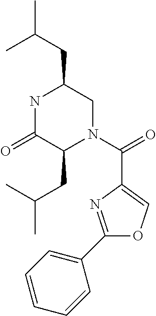

According to the method described for the synthesis of compound 89, 7 (99.8 mg, 0.47 mmol) was coupled with 2-phenyloxazole-4-carboxylic acid (106.7 mg, 0.564 mmol) to give the product 99 (12.1 mg, 6.7% yield) as a white solid.

¹H NMR (400 MHz, CD₃OD): δ 8.47 (1H, s), 8.08-8.03 (2H, m), 7.56-7.50 (3H, m), 5.39 (1H, dd, J=3.6, 14.2 Hz), 5.16-5.13 (1H, m), 3.84-3.74 (1H, m), 3.18 (1H, dd, J=11.2, 14 Hz), 1.92-1.66 (4H, m), 1.50-1.38 (2H, m), 1.08-0.92 (12H, m).

MS (ESI): m/z 384.4 [M+1]⁺.

### Example 83

### (3S,6S)-3,6-Diisobutyl-4-(5-phenyl-1H-pyrazole-3-carbonyl)piperazin-2-one (100)

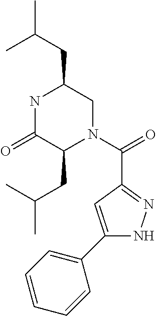

According to the method described for the synthesis of compound 89, 7 (99.8 mg, 0.47 mmol) was coupled with 5-phenyl-1H-pyrazole-3-carboxylic acid (106.1 mg, 0.564 mmol) to give the product 100 (33.4 mg, 18.6% yield) as a white solid.

¹H NMR (400 MHz, CDCl₃): δ 11.7 (1H, brs), 7.69-7.64 (2H, m), 7.45-7.35 (3H, m), 6.94 (1H, s), 6.05 (1H, s), 5.38-5.31 (1H, m), 4.88-4.79 (1H, m), 3.76-3.63 (1H, m), 3.15-3.05 (1H, m), 1.92-1.55 (4H, m), 1.43-1.28 (2H, m), 1.12-0.78 (12H, m).

MS (ESI): m/z 383.4 [M+1]⁺.

### Example 84

### (3S,6S)-3,6-Diisobutyl-4-(2-phenyl-1H-imidazole-4-carbonyl)piperazin-2-one (101)

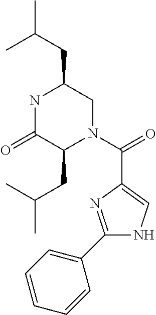

According to the method described for the synthesis of compound 89, 7 (99.8 mg, 0.47 mmol) was coupled with 2-phenyl-1H-imidazole-4-carboxylic acid (106.1 mg, 0.564 mmol) to give the product 101 (45.1 mg, 25% yield) as a white solid.

¹H NMR (400 MHz, CD₃OD): δ 7.91 (2H, d, J=6.8 Hz), 7.74 (1H, s), 7.48-7.39 (3H, m), 5.86 (1H, d, J=12 Hz), 5.20 (1H, d, J=9.2 Hz), 3.78-3.62 (1H, m), 3.17-3.11 (1H, m), 1.89-1.73 (4H, m), 1.44-1.39 (2H, m), 1.07-0.91 (12H, m).

MS (ESI): m/z 383.2 [M+1]⁺.

### Example 85

### (3S,6S)-3,6-Diisobutyl-4-(1-phenyl-1H-imidazole-4-carbonyl)piperazin-2-one (102)

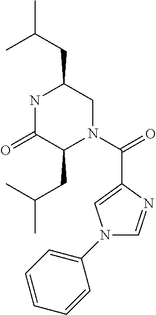

According to the method described for the synthesis of compound 89, 7 (99.8 mg, 0.47 mmol) was coupled with 1-Phenyl-1H-imidazole-4-carboxylic acid (106 mg, 0.564 mmol) to give the product 102 (82.6 mg, yield: 45.9% yield) as a colorless oil.

¹H NMR (400 MHz, CDCl₃): δ 7.99 (1H, s), 7.77 (1H, s), 7.53-7.49 (2H, m), 7.43-7.39 (3H, m), 5.94 (1H, s), 5.84-5.76 (1H, m), 5.36-5.32 (1H, m), 3.76-3.64 (1H, m), 3.10-3.04 (1H, m), 1.87-1.71 (4H, m), 1.42-1.27 (2H, m), 1.08-0.93 (12H, m).

MS (ESI): m/z 383.3 [M+1]⁺.

### Example 86

### (3S,6S)-4-(1-(4-Fluorophenyl)-1H-imidazole-4-carbonyl)-3,6-diisobutylpiperazin-2-one (103)

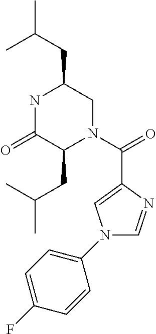

According to the method described for the synthesis of compound 89, 7 (99.8 mg, 0.47 mmol) was coupled with 1-(4-fluorophenyl)-1H-imidazole-4-carboxylic acid (116.3 mg, 0.564 mmol) to give the product 103 (28.4 mg, 15.1% yield) as a white solid.

¹H NMR (400 MHz, CDCl₃): δ 7.94 (1H, s), 7.71-7.69 (1H, m), 7.40-7.37 (2H, m), 7.23-7.19 (2H, m), 5.83-5.74 (2H, m), 5.37-5.32 (1H, m), 3.77-3.66 (1H, m), 3.10-3.04 (1H, m), 1.87-1.68 (4H, m), 1.42-1.29 (2H, m), 1.07-0.93 (12H, m).

MS (ESI): m/z 401.1 [M+1]⁺.

### Example 87

### (3S,6S)-4-(1-(4-Fluorophenyl)-1H-pyrazole-4-carbonyl)-3,6-diisobutylpiperazin-2-one (104)

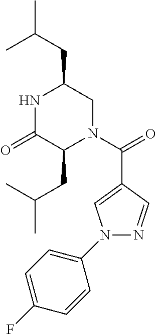

According to the method described for the synthesis of compound 89, 7 (99.8 mg, 0.47 mmol) was coupled with 1-(4-fluorophenyl)-1H-pyrazole-4-carboxylic acid (116.3 mg, 0.562 mmol) to give the product 104 (33.6 mg, 17.8% yield) as a white solid.

¹H NMR (400 MHz, CDCl₃): δ 8.22 (1H, s), 7.82 (1H, s), 7.67-7.64 (2H, m), 7.20-7.16 (2H, m), 5.78 (1H, s), 5.38-5.25 (1H, m), 4.30-4.16 (1H, m), 3.75-3.56 (1H, m), 3.30-3.15 (1H, m), 1.89-1.84 (1H, m), 1.80-1.62 (3H, m), 1.44-1.28 (2H, m) 1.12-0.74 (12H, m).

MS (ESI): m/z 401.2 [M+1]⁺.

### Example 88

### (3S,6S)-4-(3-(4-Chlorophenyl)isothiazole-5-carbonyl)-3,6-diisobutylpiperazin-2-one (105)

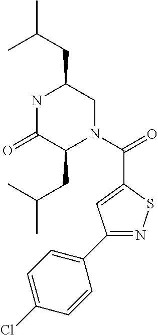

According to the method described for the synthesis of compound 89, 7 (37 mg, 0.174 mmol) was coupled with 3-(4-chlorophenyl)isothiazole-5-carboxylic acid (50 mg, 0.21 mmol) to give the product 105 (26 mg, 34.4% yield) as a colorless oil.

¹H NMR (400 MHz, CDCl₃): δ 7.88 (2H, d, J=8 Hz), 7.70 (1H, s), 7.44 (2H, d, J=8 Hz), 6.00 (1H, s), 5.32-5.23 (1H, m), 4.60-4.50 (1H, m), 3.78-3.54 (1H, m), 3.23-3.08 (1H, m), 1.95-1.83 (1H, m), 1.81-1.54 (3H, m), 1.46-1.22 (2H, m), 1.14-0.82 (12H, m).

MS (ESI): m/z 434.1 [M+1]⁺.

### Example 89

### (3S,6S)-4-(5-Cyclopropylisoxazole-3-carbonyl)-3,6-diisobutylpiperazin-2-one (106)

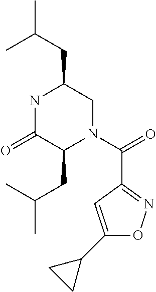

According to the method described for the synthesis of compound 89, 7 (99.8 mg, 0.47 mmol) was coupled with 5-cyclopropylisoxazole-3-carboxylic acid (86.4 mg, 0.564 mmol) to give the product 106 (122.2 mg, 74.8% yield) as a yellow oil.

¹H NMR (400 MHz, CDCl₃): δ 6.36 (1H, s), 6.25 (1H, s), 5.22 (1H, dd, J=4, 9.2 Hz), 4.75 (1H, dd, J=4, 14.2 Hz), 3.69-3.63 (1H, m), 3.04 (1H, dd, J=10.8, 14 Hz), 2.10-2.02 (1H, m), 1.88-1.62 (4H, m), 1.38-1.29 (2H, m), 1.13-1.03 (4H, m), 0.99-0.88 (12H, m).

MS (ESI): m/z 348.2 [M+1]⁺.

### Example 90

### (3S,6S)-4-(5-Ethylisoxazole-3-carbonyl)-3,6-diisobutylpiperazin-2-one (107)

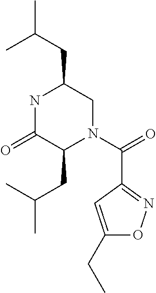

According to the method described for the synthesis of compound 89, 7 (99.8 mg, 0.47 mmol) was coupled with 5-ethylisoxazole-3-carboxylic acid (79.6 mg, 0.564 mmol) to give the product 107 (13.5 mg, yield: 8.6% yield) as a white solid.

¹H NMR (400 MHz, CD₃OD): δ 6.43 (1H, s), 5.12-5.09 (1H, m), 4.47-4.42 (1H, m), 3.73-3.62 (1H, m), 3.19-3.13 (1H, m), 2.85 (2H, q, J=7.6 Hz), 1.88-1.62 (4H, m), 1.47-1.28 (5H, m), 1.08-0.86 (12H, m).

MS (ESI): m/z 336.2 [M+1]⁺.

### Example 91

### (3S,6S)-3,6-Diisobutyl-4-(5-phenylisoxazole-3-carbonyl)piperazin-2-one (108)

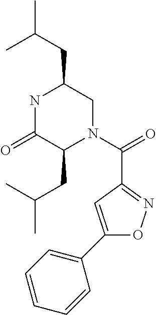

According to the method described for the synthesis of compound 89, 7 (99.8 mg, 0.47 mmol) was coupled with 5-phenylisoxazole-3-carboxylic acid (106.7 mg, 0.564 mmol) to give the product of 108 (120 mg, 66.6% yield) as a white solid.

¹H NMR (400 MHz, CDCl₃): δ 7.82-7.78 (2H, m), 7.51-7.45 (3H, m), 6.92 (1H, s), 6.36 (1H, s), 5.29-5.26 (1H, m), 4.87 (1H, dd, J=4, 14.2 Hz), 3.78-3.67 (1H, m), 3.11 (1H, dd, J=10.8, 14 Hz), 1.92-1.82 (1H, m), 1.81-1.67 (3H, m), 1.42-1.34 (2H, m), 1.08 (3H, d, J=6.4 Hz), 0.99-0.93 (9H, m).

MS (ESI): m/z 384.2 [M+1]⁺.

### Example 92

### (3S,6S)-3,6-Diisobutyl-4-(5-(thiophen-2-yl)isoxazole-3-carbonyl)piperazin-2-one (109)

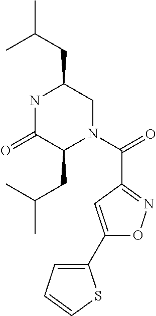

According to the method described for the synthesis of compound 89, 7 (99.8 mg, 0.47 mmol) was coupled with 5-(thiophen-2-yl)isoxazole-3-carboxylic acid (110.1 mg, 0.564 mmol) to give the product 109 (94.8 mg, 51.8% yield) as an yellow oil.

¹H NMR (400 MHz, CDCl₃): δ 7.54-7.53 (1H, m), 7.50-7.48 (1H, m), 7.15-7.13 (1H, m), 6.77 (1H, s), 6.31 (1H, s), 5.26 (1H, dd, J=4.4, 9.2 Hz), 4.83 (1H, dd, J=4.4, 14 Hz), 3.77-3.64 (1H, m), 3.10 (1H, dd, J=10.8, 14 Hz), 1.91-1.66 (4H, m), 1.41-1.26 (2H, m), 1.08-0.90 (12H, m).

MS (ESI): m/z 390.4 [M+1]⁺.

### Example 93

### (3S,6S)-3-Isobutyl-6-phenyl-4-(5-(thiophen-2-yl)isoxazole-3-carbonyl)piperazin-2-one (110)

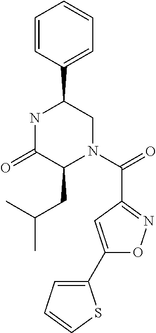

According to the method described for the synthesis of compound 89, 11 (50 mg, 0.22 mmol) was coupled with 5-(thiophen-2-yl)isoxazole-3-carboxylic acid (50.4 mg, 0.258 mmol) to give the product 110 (63.2 mg, 70.1% yield) as pale yellow solid.

¹H NMR (400 MHz, CDCl₃): δ 7.57-7.50 (2H, m), 7.44-7.37 (5H, m), 7.17-7.14 (1H, m), 6.80 (1H, s), 6.22 (1H, s), 5.38-5.34 (1H, m), 4.94-4.87 (2H, m), 3.37-3.29 (1H, m), 2.00-1.70 (3H, m), 1.10 (3H, d, J=6.8 Hz), 0.99 (3H, d, J=6.8 Hz).

MS (ESI): m/z 410.2 [M+1]⁺.

### Example 94

### (3S,6S)-3-Isobutyl-6-isopropyl-4-(5-(thiophen-2-yl)isoxazole-3-carbonyl)piperazin-2-one (111)

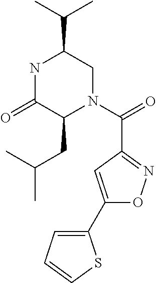

According to the method described for the synthesis of compound 89, 8 (42.3 mg, 0.21 mmol) was coupled with 5-(thiophen-2-yl)isoxazole-3-carboxylic acid (50 mg, 0.26 mmol) to give the product 111 (52.6 mg, 66.7% yield) as a pink oil.

¹H NMR (400 MHz, CDCl₃): δ 7.55-7.54 (1H, m), 7.51-7.48 (1H, m), 7.16-7.13 (1H, m), 6.77 (1H, s), 6.02 (1H, s), 5.29-5.25 (1H, m), 4.88-4.84 (1H, m), 3.53-3.42 (1H, m), 3.22-3.16 (1H, m), 1.90-1.66 (4H, m), 1.08-0.97 (12H, m).

MS (ESI): m/z 376.1 [M+1]⁺.

### Example 95

### (3S,6S)-4-(5-(5-Chlorothiophen-2-yl)isoxazole-3-carbonyl)-3,6-diisobutylpiperazin-2-one (112)

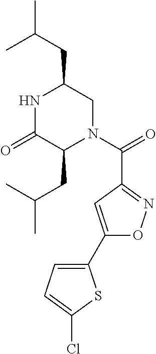

According to the method described for the synthesis of compound 89, Compound 7 (99.8 mg, 0.47 mmol) was coupled with 5-(5-chlorothiophen-2-yl)isoxazole-3-carboxylic acid (129.5 mg, 0.564 mmol) to give the product 112 (89.5 mg, 44.9% yield) as an oil.

¹H NMR (400 MHz, CDCl₃): δ 7.31 (1H, d, J=4 Hz), 6.97 (1H, d, J=4 Hz), 6.73 (1H, s), 6.15 (1H, s), 5.27-5.24 (1H, m), 4.84-4.79 (1H, m), 3.77-3.66 (1H, m), 3.10 (1H, dd, J=10.8, 14 Hz), 1.91-1.67 (4H, m), 1.41-1.33 (2H, m), 1.07-0.91 (12H, m).

MS (ESI): m/z 424.1 [M+1]⁺.

### Example 96

### (3S,6S)-4-(3-(4-fluorophenyl)propioloyl)-3,6-diisobutylpiperazin-2-one (113)

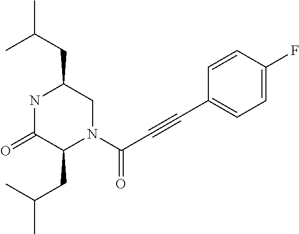

According to the method described for the synthesis of compound 89, Compound 7 (50 mg, 0.235 mmol) and 3-(4-fluorophenyl)propiolic acid (46.3 mg, 0.282 mmol) were coupled to give the product 113 (59 mg, 70% yield) as a white solid.

¹H NMR (400 MHz, CDCl₃): δ 7.56-7.51 (2H, m), 7.11-7.05 (2H, m), 5.98 (1H, s), 4.92-4.89 (1H, m), 4.67 (1H, dd, J=4.4, 13.4 Hz), 3.68-3.57 (1H, m), 2.71 (1H, dd, J=11.2, 13.6 Hz), 1.88-1.62 (4H, m), 1.40-1.34 (2H, m), 1.06-0.93 (12H, m).

MS (ESI): m/z 359.1 [M+1]⁺.

### Example 97

### 3,6-Diisobutyl-4-(3-phenyl-propynoyl)-piperazin-2-one (114)

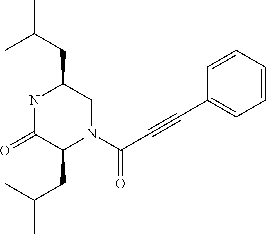

A mixture of Compound 7 (400 mg, 1.88 mmol), phenyl-propynoic acid (275 mg, 1.88 mmol), HATU (714 mg, 1.88 mmol) and Et₃N (500 mg, 4.95 mmol) in DCM (10 mL) were stirred at 25° C. for 12 h. The mixture was washed with H₂O (10 mL), dried with MgSO₄, filtered, evaporated to dryness and purified by prep-HPLC to afford desired product 114 (195 mg, 30.42%) as a white solid.

¹H NMR (400 MHz, DMSO-d6): δ 7.51-7.55 (m, 2H), 7.34-7.45 (m, 3H), 6.10-6.14 (m, 1H), 5.08-5.18 (m, 0.4H), 4.92-5.15 (m, 0.6H), 4.64-4.70 (m, 0.6H), 4.64-4.70 (m, 0.4H), 3.59-3.67 (m, 1H), 3.12 (dd, J=13.6, 11.2 Hz, 0.4H), 2.67-2.73 (dd, J=13.6, 11.2 Hz, 0.6H), 1.78-1.86 (m, 2H), 1.65-1.73 (m, 2H), 1.34-1.40 (m, 2H), 0.90-1.06 (m, 12H).

MS (ESI): m/z 341.1 [M+1]⁺.

### Example 98

### 3,6-Diisobutyl-4-(3-p-tolyl-propynoyl)-piperazin-2-one (115)

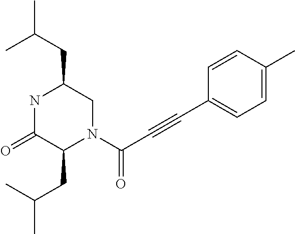

Compound 7 (150 mg, 0.7 mmol) and p-Tolyl-propynoic acid (112 mg, 0.7 mmol) were coupled according to the method described for compound 114 to give the product 115 (86 mg, 34.44% yield) as a white solid.

¹H NMR (400 MHz, CDCl₃): δ 7.36 (t, J=8.0 Hz, 2H), 7.11 (t, J=6.0 Hz, 2H), 6.08 (d, J=11.2 Hz, 1H), 4.89 (d, J=3.6 Hz, 0.4H), 4.87 (d, J=2.8 Hz, 0.6H), 4.63 (d, J=4.0 Hz, 0.6H), 4.60 (d, J=4.4 Hz, 0.4H), 3.54 (q, J=10.8 Hz, 1H), 3.05 (dd, J=14.0 Hz, 11.2 Hz, 0.4H), 2.64 (dd, J=13.6 Hz, 11.2 Hz, 0.6H), 2.31 (s, 3H), 1.77 (d, J=4.8 Hz, 1H), 1.67 (d, J=8.4 Hz, 1H), 1.30 (dd, J=10.8 Hz, 7.2 Hz, 2H), 0.93 (m, 12H);

MS (ESI): m/z 355.2 [M+1]⁺

### Example 99

### 4-[3-(3,4-Difluoro-phenyl)-propynoyl]-3,6-diisobutyl-piperazin-2-one (116)

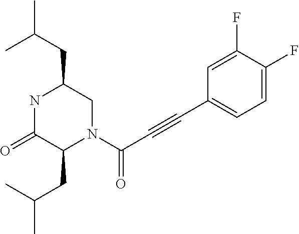

Compound 7 (150 mg, 0.7 mmol) and (3,4-Difluoro-phenyl)-propynoic acid (127 mg, 0.7 mmol) were coupled according to the method described for compound 114 to give the product 116 (98.4 mg, 37.3% yield) as a white solid.

¹H NMR (400 MHz, CDCl₃) δ 7.32 (m, 2H), 7.18 (m, 1H), 5.78 (s, 1H), 5.12 (d, J=3.6 Hz, 0.4H), 4.86 (d, J=2.8 Hz, 0.6H), 4.63 (d, J=4.0 Hz, 0.6H), 4.39 (d, J=4.4 Hz, 0.4H), 3.56 (q, J=10.8 Hz, 1H), 3.11 (dd, J=14.0 Hz, 11.2 Hz, 0.4H), 2.71 (dd, J=13.6 Hz, 11.2 Hz, 0.6H), 1.72 (s, 4H), 1.36 (dd, J=10.8 Hz, 7.2 Hz, 2H), 0.93 (m, 12H).

MS (ESI): m/z 753.0 [2M+1]⁺

### Example 100

### 4-[3-(2,4-Difluoro-phenyl)-propynoyl]-3,6-diisobutyl-piperazin-2-one (117)

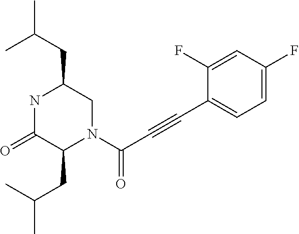

Compound 7 (150 mg, 0.71 mmol) and (2,4-Difluoro-phenyl)-propynoic acid (155 mg, 0.85 mmol) were coupled according to the method described for compound 114 to give the product 117 (56 mg, 22.4% yield) as a white solid.

¹H NMR (400 MHz, CDCl₃): δ 7.49 (m, 1H), 6.84 (m, 2H), 5.98 (s, 1H), 5.08 (d, J=3.6 Hz, 0.5H), 4.86 (d, J=2.8 Hz, 0.5H), 4.63 (d, J=4.0 Hz, 0.5H), 4.47 (d, J=4.4 Hz, 0.5H), 3.58 (q, J=10.8 Hz, 1H), 3.11 (dd, J=14.0 Hz, 11.2 Hz, 0.5H), 2.66 (dd, J=13.6 Hz, 11.2 Hz, 0.5H), 1.76 (m, 1H), 1.62 (m, 1H), 1.31 (m, 2H), 0.93 (m, 12H).

MS (ESI): m/z 377.2 [M+1]⁺

### Example 101

### 3,6-Diisobutyl-4-[3-(4-methoxy-phenyl)-propynoyl]-piperazin-2-one (118)

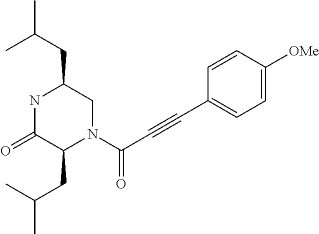

Compound 7 (150 mg, 0.7 mmol) and (4-Methoxy-phenyl)-propynoic acid (123.2 mg, 0.7 mmol) were coupled according to the method described for compound 114 to give the product 118 (115.8 mg, 44.7% yield) as a white solid.

¹H NMR (400 MHz, CDCl₃): δ 7.47 (d, J=9.2 Hz, 2H), 6.88 (dd, J=6.8 Hz, 4.8 Hz, 2H), 5.69 (d, J=17.6 Hz, 1H), 5.14 (d, J=8.0 Hz, 0.4H), 4.94 (d, J=8.0 Hz, 0.6H), 4.69 (d, J=8.0 Hz, 0.6H), 4.65 (d, J=8.0 Hz, 0.4H), 3.83 (s, 3H), 3.59 (q, J=10.8 Hz, 1H), 3.11 (dd, J=14.0 Hz, 11.2 Hz, 0.4H), 2.71 (dd, J=13.6 Hz, 11.2 Hz, 0.6H), 1.84 (dd, J=10.0 Hz, 4.8 Hz, 2H), 1.69 (m, 2H), 1.36 (dd, J=10.8 Hz, 7.2 Hz, 2H), 0.93 (m, 12H);

MS (ESI): m/z 371.3 [M+1]⁺

### Example 102

### 4-[3-(4-Chloro-2-fluoro-phenyl)-propynoyl]-3,6-diisobutyl-piperazin-2-one (119)

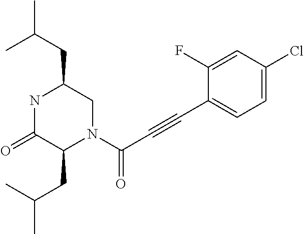

Compound 7 (150 mg, 0.7 mmol) and (4-Chloro-2-fluoro-phenyl)-propynoic acid (168.3 mg, 0.85 mmol) were coupled according to the method described for compound 114 to give the product 119 (112 mg, 40.4% yield) as a white solid.

¹H NMR (400 MHz, CDCl₃): δ 7.43 (m, 1H), 7.11 (d, J=8.4 Hz, 2H), 5.91 (d, J=7.2 Hz, 1H), 5.08 (d, J=3.6 Hz, 0.5H), 4.86 (d, J=2.8 Hz, 0.5H), 4.63 (d, J=4.0 Hz, 0.5H), 4.47 (d, J=4.4 Hz, 0.5H), 3.58 (dd, J=10.8 Hz, 1H), 3.07 (dd, J=14.0 Hz, 11.2 Hz, 0.5H), 2.66 (dd, J=13.6 Hz, 11.2 Hz, 0.5H), 1.76 (m, 1H), 1.64 (m, 2H), 1.31 (m, J=7.2 Hz, 2H), 0.93 (m, 12H);

MS (ESI): m/z 393.0 [M+1]⁺

### Example 103

### 4-[3-(4-Chloro-phenyl)-propynoyl]-3,6-diisobutyl-piperazin-2-one (120)

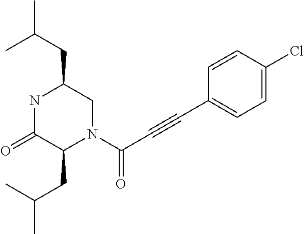

Compound 7 (150 mg, 0.71 mmol) and (4-Chloro-phenyl)-propynoic acid (153 mg, 0.85 mmol) were coupled according to the method described for compound 114 to give the product 120 (164 mg, 61.9% yield) as a white solid.

¹H NMR (400 MHz, CDCl₃) δ 7.40 (t, J=7.8 Hz, 2H), 7.30 (dd, J=6.4, 4.4 Hz, 2H), 6.15 (d, J=28.4 Hz, 1H), 5.06 (d, J=9.2 Hz, 0.4H), 4.81 (dd, J=4.4, 1.2 Hz, 0.6H), 4.61 (dd, J=13.2, 4.0 Hz, 0.6H), 4.39 (dd, J=14.0, 4.0 Hz, 0.4H), 3.54 (d, J=6.8 Hz, 1H), 3.07 (d, J=13.6 Hz, 1H), 2.65 (dd, J=13.6, 10.8 Hz, 0.4H), 1.77 (t, J=4.8 Hz, 2H), 1.66 (dd, J=12.8 Hz, 6.8 Hz, 4H), 1.31 (d, J=6.8 Hz, 2H), 0.89 (m, 12H);

MS (ESI): m/z 375.0 [M+1]⁺

### Example 104

### (3S,6S)-3,6-Diisobutyl-4-[3-(3,4,5-trifluoro-phenyl)-propynoyl]-piperazin-2-one(121)

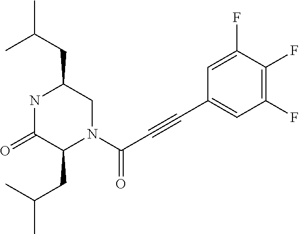

Compound 7 (84.8 mg, 0.4 mmol) and (3,4,5-Trifluoro-phenyl)-propynoic acid (80 mg, 0.4 mmol) were coupled according to the method described for compound 114 to give the product 121 (95 mg, 60% yield) as a white solid.

¹H NMR (CDCl₃) δ 7.14-7.24 (m, 2H), 5.69-5.72 (d, J=11.6 Hz, 1H), 5.10-5.14 (dd, J=3.2, 9.6 Hz 0.4H), 4.82-4.88 (dd, J=3.2, 9.6 Hz 0.6H), 4.61-4.69 (dd, J=4.4, 13.6 Hz, 0.6H), 4.37-4.43 (dd, J=4.4, 13.6 Hz, 0.4H), 3.54-3.72 (m, 1H), 3.08-3.16 (m, 0.4H), 2.65-2.73 (m, 0.6H), 1.65-1.86 (m, 4H), 1.39-1.43 (m, 2H), 0.92-1.06 (m, 12H).

MS (ESI): m/z 395.0 [M+1]⁺.

### Example 105

### (3S,6S)-3,6-Diisobutyl-4-(3-(4-(trifluoromethyl)phenyl)-propioloyl)piperazin-2-one (122)

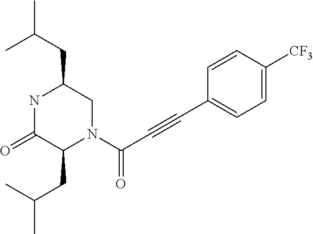

According to the method described for compound 89, Compound 7 (65.5 mg, 0.31 mmol) was coupled with 3-(4-(trifluoromethyl)phenyl)propiolic acid (78.7 mg, 0.37 mmol) to give the product 122 (51.2 mg, 40.4% yield) as a white solid.

¹H NMR (400 MHz, CDCl₃): δ 7.67-7.63 (4H, m), 6.03 (1H, s), 4.91-4.87 (1H, m), 4.68 (1H, d, J=4, 13.4 Hz), 3.71-3.58 (1H, m), 2.73 (1H, d, J=11, 13.4 Hz), 1.89-1.61 (4H, m), 1.41-1.34 (2H, m), 1.05-0.93 (12H, m).

MS (ESI): m/z 409.2 [M+1]⁺.

### Example 106

### (3S,6S)-4-(5-(3,4-Difluorophenyl)isoxazole-3-carbonyl)-3,6-diisobutylpiperazin-2-one (123)

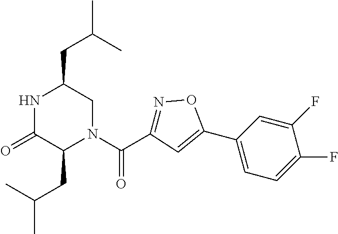

A mixture of Compound 7 (45 mg, 0.21 mmol), 35 (56 mg, 0.25 mmol), HOBT (35 mg, 0.26 mmol), EDC (48 mg, 0.25 mmol), DIPEA (0.07 mL, 0.42 mmol) in DMF (3 mL) was stirred at room temperature overnight. After removal of solvents, the residue was purified by column to give the product 123 (57 mg, 65% yield) as a white solid.

¹H NMR (400 MHz, CD₃OD): δ 7.65-7.60 (1H, m), 7.55-7.53 (1H, m), 7.33-7.26 (1H, m), 6.89 (1H, s), 5.93 (1H, s), 5.28 (1H, dd, J=3.8, 9.8 Hz), 4.86 (1H, dd, J=3.8, 14 Hz), 3.79-3.69 (1H, m), 3.12 (1H, dd, J=10.8, 14 Hz), 1.92-1.85 (1H, m), 1.79-1.65 (3H, m), 1.42-1.32 (2H, m), 1.08-0.92 (12H, m).

MS (ESI): m/z 420.2 [M+1]⁺.

### Example 107

### (3S,6S)-4-(5-(4-Fluorophenyl)isoxazole-3-carbonyl)-3,6-diisobutylpiperazin-2-one (124)

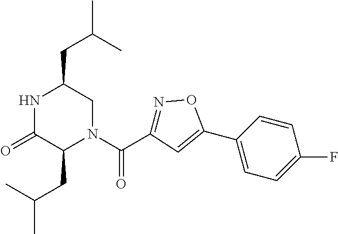

According to the method described for compound 123, 7 (853.3 mg, 4.025 mmol) was coupled with 5-(4-fluorophenyl)isoxazole-3-carboxylic acid (1000 mg, 4.23 mmol) to give the product 124 (72.1 mg, yield: 4.5% yield) as a white solid.

¹H NMR (400 MHz, CD₃OD): δ 7.96-7.92 (2H, m), 7.30-7.26 (2H, m), 7.06 (1H, s), 5.16-5.12 (1H, m), 4.53 (1H, dd, J=4.4, 14.2 Hz), 3.79-3.71 (1H, m), 3.21 (1H, dd, J=11, 14.2 Hz), 1.92-1.62 (4H, m), 1.48-1.32 (2H, m), 1.12-0.88 (12H, m).

MS (ESI): m/z 402.2 [M+1]⁺.

### Example 108

### (3S,6S)-4-(5-(4-Chlorophenyl)isoxazole-3-carbonyl)-3,6-diisobutylpiperazin-2-one (125)

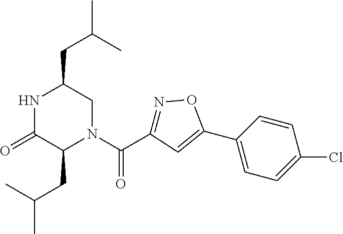

According to the method described for compound 123, 7 (99.8 mg, 0.47 mmol) was coupled with 5-(4-chlorophenyl)isoxazole-3-carboxylic acid (126.1 mg, 0.564 mmol) to give the product 125 (109.1 mg, 55.5% yield) as a white solid.

¹H NMR (400 MHz, CDCl₃): δ 7.72 (2H, d, J=8.4 Hz), 7.46 (2H, d, J=8.4 Hz), 6.91 (1H, s), 6.49 (1H, s), 5.26 (1H, dd, J=4, 9.4 Hz), 4.85 (1H, dd, J=4, 14.2 Hz), 3.77-3.67 (1H, m), 3.10 (1H, dd, J=11, 14.2 Hz), 1.91-1.67 (4H, m), 1.41-1.34 (2H, m), 1.07-0.91 (12H, m).

MS (ESI): m/z 418.4 [M+1]⁺.

### Example 109

### (3S,6S)-3,6-Diisobutyl-4-(5-(p-tolyl)isoxazole-3-carbonyl)piperazin-2-one (126)

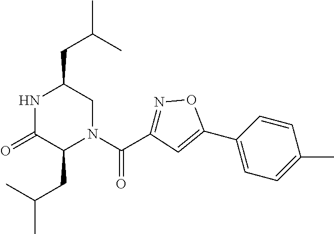

According to the method described for compound 123, 7 (99.8 mg, 0.47 mmol) was coupled with 5-(p-tolyl)isoxazole-3-carboxylic acid (114.6 mg, 0.564 mmol) to give the product 126 (107 mg, 57.3% yield) as a white solid.

¹H NMR (400 MHz, CDCl₃): δ 7.67 (2H, d, J=8 Hz), 7.27 (2H, d, J=8 Hz), 6.85 (1H, s), 6.51 (1H, s), 5.26 (1H, dd, J=4, 9.4 Hz), 4.86 (1H, dd, J=4, 14.2 Hz), 3.77-3.67 (1H, m), 3.10 (1H, dd, J=11, 14.2 Hz), 2.40 (3H, s), 1.91-1.67 (4H, m), 1.41-1.33 (2H, m), 1.08-0.91 (12H, m).

MS (ESI): m/z 398.2 [M+1]⁺.

### Example 110

### (3S,6S)-3,6-Diisobutyl-4-(5-(4-methoxyphenyl)isoxazole-3-carbonyl)piperazin-2-one (127)

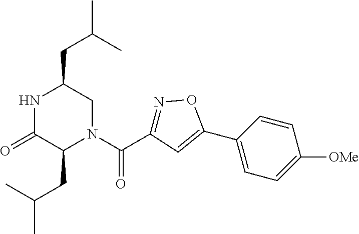

According to the method described for compound 123, 7 (99.8 mg, 0.47 mmol) was coupled with 5-(4-methoxyphenyl)isoxazole-3-carboxylic acid (123.6 mg, 0.564 mmol) to give the product 127 (105.8 mg, yield: 54.5%) as a white solid.

¹H NMR (400 MHz, CDCl₃): δ 7.72 (2H, d, J=8.8 Hz), 6.98 (2H, d, J=8.8 Hz), 6.78 (1H, s), 6.51 (1H, s), 5.26 (1H, dd, J=4.4, 9.4 Hz), 4.85 (1H, dd, J=4, 14 Hz), 3.85 (3H, s), 3.77-3.66 (1H, m), 3.09 (1H, dd, J=10.8, 13.8 Hz), 1.91-1.67 (4H, m), 1.41-1.33 (2H, m), 1.07-0.90 (12H, m).

MS (ESI): m/z 414.1 [M+1]⁺.

### Example 111

### (3S,6S)-4-(5-(3-Fluorophenyl)isoxazole-3-carbonyl)-3,6-diisobutylpiperazin-2-one (128)

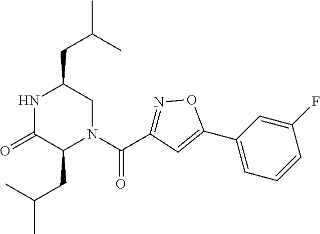

According to the method described for compound 123, 7 (99.8 mg, 0.47 mmol) was coupled with 5-(3-fluorophenyl)isoxazole-3-carboxylic acid 37 (116.8 mg, 0.564 mmol) to give the product 128 (108 mg, 57.2% yield) as a white foam.

¹H NMR (400 MHz, CDCl₃): δ 7.57 (1H, d, J=7.6 Hz), 7.51-7.44 (2H, m), 7.19-7.15 (1H, m), 6.94 (1H, s), 6.24 (1H, s), 5.27-5.25 (1H, m), 4.86-4.82 (1H, m), 3.78-3.65 (1H, m), 3.11 (1H, dd, J=11.2, 13.6 Hz), 1.92-1.69 (4H, m), 1.42-1.34 (2H, m), 1.08-0.91 (12H, m).

MS (ESI): m/z 402.2 [M+1]⁺.

### Example 112

### (3S,6S)-4-(5-(3-Chlorophenyl)isoxazole-3-carbonyl)-3,6-diisobutylpiperazin-2-one (129)

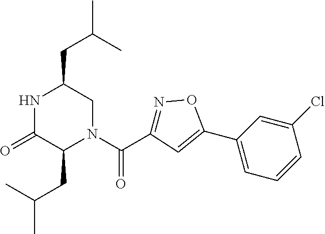

According to the method described for compound 123, 7 (99.8 mg, 0.47 mmol) was coupled with 5-(3-chlorophenyl)isoxazole-3-carboxylic acid 38 (126 mg, 0.564 mmol) to give the product 129 (98.9 mg, 50.3% yield) as a white solid.

¹H NMR (400 MHz, CDCl₃): δ 7.79 (1H, s), 7.67-7.66 (1H, m), 7.46-7.41 (2H, m), 6.95 (1H, s), 6.36 (1H, s), 5.28-5.25 (1H, m), 4.86-4.82 (1H, m), 3.78-3.66 (1H, m), 3.11 (1H, dd, J=11.2, 13.6 Hz), 1.92-1.69 (4H, m), 1.42-1.34 (2H, m), 1.08-0.91 (12H, m).

MS (ESI): m/z 418.2 [M+1]⁺.

### Example 113

### (3S,6S)-3,6-Diisobutyl-4-(5-(3-methoxyphenyl)isoxazole-3-carbonyl)piperazin-2-one (130)

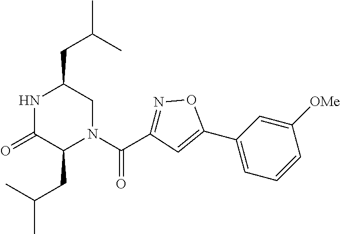

According to the method described for compound 123, 7 (99.8 mg, 0.47 mmol) was coupled with 5-(3-methoxyphenyl)isoxazole-3-carboxylic acid 39 (123.6 mg, 0.564 mmol) to give the product 130 (67 mg, 34.5% yield) as a pale yellow solid.

¹H NMR (400 MHz, CDCl₃): δ 7.42-7.32 (3H, m), 7.03-6.99 (1H, m), 6.91 (1H, s), 6.15 (1H, s), 5.32-5.27 (1H, m), 4.87 (1H, dd, J=4, 14 Hz), 3.87 (3H, s), 3.79-3.66 (1H, m), 3.11 (1H, dd, J=11.2, 14 Hz), 1.92-1.66 (4H, m), 1.42-1.34 (2H, m), 1.08-0.91 (12H, m).

MS (ESI): m/z 414.1 (M+H)⁺.

### Example 114

### (3S,6S)-3,6-diisobutyl-4-(5-(2-methoxyphenyl)isoxazole-3-carbonyl)piperazin-2-one (131)

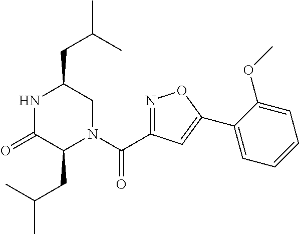

Compound (7) (50 mg, 0.24 mmol) and 5-(2-methoxyphenyl)isoxazole-3-carboxylic acid 40 (52 mg, 0.24 mmol) were coupled according to the procedure described for compound 71 to furnish 131 (69 mg, 71% yield) as a white solid.

¹H NMR (400 MHz, CDCl₃): δ 7.99-7.97 (m, 1H), 7.46-7.41 (m, 1H), 7.17-7.01 (m, 3H), 5.97 (s, 1H), 5.32-5.26 (m, 1H), 4.87-4.79 (m, 1H), 3.96 (s, 3H), 3.80-3.69 (m, 1H), 3.13-2.82 (m, 1H), 1.92-1.42 (m, 4H), 1.40-1.30 (m, 2H), 1.09-0.76 (m, 12H);

MS (ESI): m/z 414.1 (M+1)⁺

### Example 115

### (3S,6S)-4-(5-(4-(tert-butyl)phenyl)isoxazole-3-carbonyl)-3,6-diisobutylpiperazin-2-one (132)

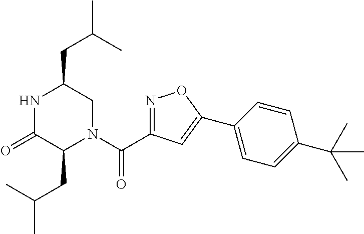

Compound 7 (50 mg, 0.24 mmol) and 5-(4-(tert-butyl)phenyl)isoxazole-3-carboxylic acid 41 (58 mg, 0.24 mmol) were coupled according to the procedure described for compound 71 to furnish 132 (89 mg, 86% yield) as a white solid.

¹H NMR (400 MHz, CDCl₃): δ 7.74-7.71 (m, 2H), 7.52-7.49 (m, 2H), 6.87 (s, 1H), 5.83 (s, 1H), 5.34-5.28 (m, 1H), 4.91-4.78 (m, 1H), 3.77-3.69 (m, 1H), 3.14-2.83 (m, 1H), 1.92-1.83 (m, 1H), 1.80-1.66 (m, 3H), 1.42-1.29 (m, 2H), 1.09-0.76 (m, 12H);

MS (ESI): m/z 440.2 (M+1)⁺.

### Example 116

### (3S,6S)-4-(5-(2,4-difluorophenyl)isoxazole-3-carbonyl)-3,6-diisobutylpiperazin-2-one (133)

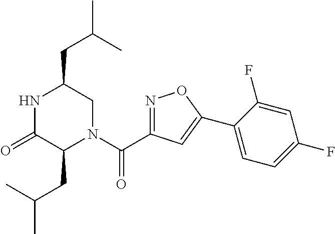

Compound (7) (50 mg, 0.24 mmol) and 5-(2,4-difluorophenyl)isoxazole-3-carboxylic acid (53 mg, 0.24 mmol) were coupled according to the procedure described for compound 71 to furnish 133 (72 mg, 73% yield) as a white solid.

¹H NMR (400 MHz, CDCl₃): δ 8.00-7.93 (m, 1H), 7.07-6.95 (m, 3H), 5.88 (s, 1H), 5.30-5.22 (m, 1H), 4.81-4.76 (m, 1H), 3.78-3.70 (m, 1H), 3.15-2.79 (m, 1H), 1.89-1.61 (m, 4H), 1.42-1.31 (m, 2H) 1.09-0.76 (m, 12H);

MS (ESI): m/z 420.1 (M+1)⁺

### Example 117

### (3S,6S)-4-(5-(4-chloro-3-fluorophenyl)isoxazole-3-carbonyl)-3,6-diisobutylpiperazin-2-one (134)

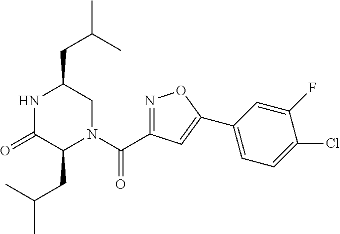

Compound (7) (50 mg, 0.24 mmol) and 5-(4-chloro-3-fluorophenyl)isoxazole-3-carboxylic acid 51 (59 mg, 0.24 mmol) were coupled according to the procedure described for compound 71 to furnish 134 (78 mg, 76% yield) as a white solid.

¹H NMR (400 MHz, CDCl₃): δ 7.59-7.50 (m, 3H), 7.21-7.16 (m, 2H), 6.94 (s, 1H), 5.94 (s, 1H), 5.29-5.26 (m, 1H), 4.87-4.76 (m, 1H), 3.77-3.69 (m, 1H), 3.15-2.82 (m, 1H), 1.92-1.83 (m, 1H), 1.79-1.65 (m, 3H), 1.42-1.30 (m, 2H), 1.08-0.77 (m, 12H);

MS (ESI): m/z 436.1 (M+1)⁺.

### Example 118

### (3S,6S)-4-(5-(4-Bromophenyl)isoxazole-3-carbonyl)-3,6-diisobutylpiperazin-2-one (135)

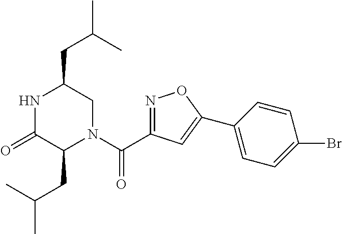

According to the method described for compound 123, 7 (99.8 mg, 0.47 mmol) was coupled with 5-(4-bromophenyl)isoxazole-3-carboxylic acid (151.2 mg, 0.564 mmol) to give the product 135 (130.1 mg, yield: 59.9%) as an off-white solid.

¹H NMR (400 MHz, CDCl3): δ 7.66-7.60 (4H, m), 6.92 (1H, s), 6.54 (1H, s), 5.27-5.24 (1H, m), 4.86-4.76 (1H, m), 3.77-3.66 (1H, m), 3.10 (1H, dd, J=11.2, 13.6 Hz), 1.91-1.69 (4H, m), 1.41-1.34 (2H, m), 1.07-0.91 (12H, m).

MS (ESI): m/z 462.1 [M+1]⁺.

### Example 119

### 4-(3-((2S,5S)-2,5-Diisobutyl-3-oxopiperazine-1-carbonyl)isoxazol-5-yl)benzonitrile (136)

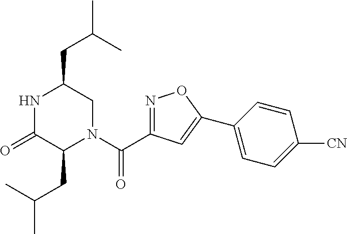

According to the method described for compound 123, 7 (99.8 mg, 0.47 mmol) was coupled with 5-(4-cyanophenyl)isoxazole-3-carboxylic acid 49 (121.2 mg, 0.564 mmol) to give the product 136 (60.6 mg, 31.6% yield) as an off-white solid.

¹H NMR (400 MHz, CDCl₃): δ 7.91 (2H, d, J=8.4 Hz), 7.79 (2H, d, J=8.4 Hz), 7.07 (1H, s), 6.32 (1H, s), 5.30-5.24 (1H, m), 4.87-4.83 (1H, m), 3.79-3.67 (1H, m), 3.12 (1H, dd, J=11.2, 13.6 Hz), 1.92-1.67 (4H, m), 1.42-1.35 (2H, m), 1.08-0.90 (12H, m).

MS (ESI): m/z 409.2 [M+1]⁺.

### Example 120

### 4-(3-((2S,5S)-5-Cyclopentyl-2-isobutyl-3-oxopiperazine-1-carbonyl)isoxazol-5-yl)benzonitrile (137)

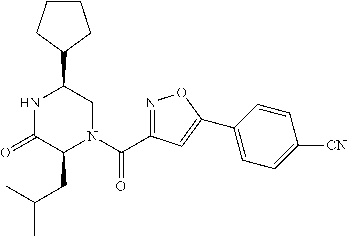

According to the method described for compound 123, 17 (93.7 mg, 0.42 mmol) was coupled with 5-(4-cyanophenyl)isoxazole-3-carboxylic acid 49 (107.4 mg, 0.50 mmol) to give the product 137 (69.1 mg, 39.3% yield) as a pale yellow oil.

¹H NMR (400 MHz, CDCl₃): δ 7.91 (2H, d, J=8.4 Hz), 7.79 (2H, d, J=8.4 Hz), 7.07 (1H, s), 6.09 (1H, s), 5.28-5.25 (1H, m), 4.92-4.88 (1H, m), 3.53-3.43 (1H, m), 3.17 (1H, dd, J=11.2, 13.6 Hz), 1.92-1.54 (10H, m), 1.36-1.22 (2H, m), 1.07 (3H, d, J=6.4 Hz), 0.98 (3H, d, J=6.4 Hz).

MS (ESI): m/z 421.2 [M+1]⁺.

### Example 121

### (3S,6S)-3,6-Diisobutyl-4-(5-(3-(trifluoromethoxy)phenyl)isoxazole-3-carbonyl)piperazin-2-one (138)

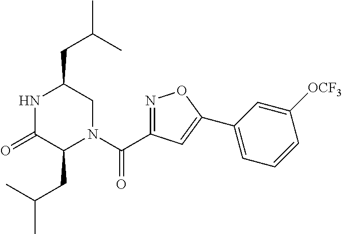

According to the method described for compound 123, 7 (32 mg, 0.15 mmol) was coupled with 5-(3-(trifluoromethoxy)phenyl)isoxazole-3-carboxylic acid 50 (50 mg, 0.18 mmol) to give the product 138 (41 mg, 58.5% yield) as colorless oil.

¹H NMR (400 MHz, CDCl₃): δ 7.74-7.66 (2H, m), 7.56-7.52 (1H, m), 7.34-7.32 (1H, m), 6.98 (1H, s), 6.00 (1H, s), 5.30-5.27 (1H, m), 4.89-4.84 (1H, m), 3.80-3.69 (1H, m), 3.15-3.09 (1H, m), 1.92-1.66 (4H, m), 1.42-1.30 (2H, m), 1.09-0.92 (12H, m).

MS (ESI): m/z 468.2 [M+1]⁺.

### Example 122

### 4-(3-((2S,5S)-2,5-Diisobutyl-3-oxopiperazine-1-carbonyl)isoxazol-5-yl)-N,N-dimethylbenzamide (139)

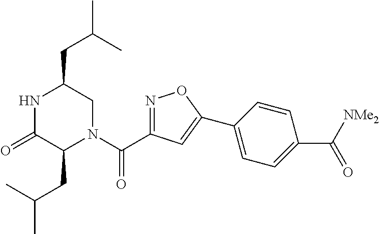

According to the method described for compound 123, 7 (99.8 mg, 0.47 mmol) was coupled with 5-(4-(dimethylcarbamoyl)phenyl)isoxazole-3-carboxylic acid 48 (147.3 mg, 0.566 mmol) to give the product 139 (56.4 mg, 26.4% yield) as off-white solid.

¹H NMR (400 MHz, CDCl₃): δ 7.82 (2H, d, J=8.4 Hz), 7.53 (2H, d, J=8.4 Hz), 6.96 (1H, s), 6.35 (1H, s), 5.28-5.24 (1H, m), 4.86-4.82 (1H, m), 3.78-3.64 (1H, m), 3.16-3.07 (1H, m), 3.12 (3H, s), 2.99 (3H, s), 1.91-1.68 (4H, m), 1.41-1.33 (2H, m), 1.07-0.90 (12H, m). MS (ESI): m/z 455.2 [M+1]⁺.

### Example 123

### (3S,6S)-4-(5-(4-(Dimethylamino)phenyl)isoxazole-3-carbonyl)-3,6-diisobutylpiperazin-2-one (140)

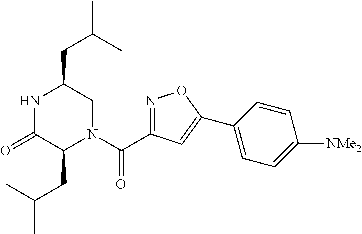

According to the method described for compound 123, 7 (99.8 mg, 0.47 mmol) was coupled with 5-(4-(dimethylamino)phenyl)isoxazole-3-carboxylic acid 43 (131.1 mg, 0.564 mmol) to give the product 140 (52.2 mg, 26% yield) as white solid.

¹H NMR (400 MHz, CDCl₃): δ 7.64 (2H, d, J=8.8 Hz), 6.72 (2H, d, J=8.8 Hz), 6.67 (1H, s), 6.43 (1H, s), 5.29-5.25 (1H, m), 4.87-4.81 (1H, m), 3.78-3.65 (1H, m), 3.08 (1H, dd, J=10.8, 13.6 Hz), 3.03 (6H, s), 1.90-1.67 (4H, m), 1.41-1.32 (2H, m), 1.08-0.90 (12H, m).

MS (ESI): m/z 455.2 427.2 [M+1]⁺.

### Example 124

### (3S,6S)-4-(5-(3-Chloro-4-fluorophenyl)isoxazole-3-carbonyl)-3,6-diisobutylpiperazin-2-one (141)

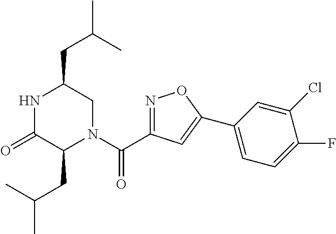

According to the method described for compound 123, 7 (99.8 mg, 0.47 mmol) was coupled with 5-(3-chloro-4-fluorophenyl)isoxazole-3-carboxylic acid 34 (136.3 mg, 0.564 mmol) to give the product 141 (107.1 mg, 52.3% yield) as a white solid.

¹H NMR (400 MHz, CDCl₃): δ 7.87-7.84 (1H, m), 7.68-7.64 (1H, m), 7.28-7.24 (1H, m), 6.90 (1H, s), 6.48 (1H, s), 5.27-5.24 (1H, m), 4.86-4.76 (1H, m), 3.77-3.65 (1H, m), 3.10 (1H, dd, J=11.2, 13.6 Hz), 1.91-1.67 (4H, m), 1.42-1.34 (2H, m), 1.07-0.91 (12H, m).

MS (ESI): m/z 436.1 [M+1]⁺.

### Example 125

### (3S,6S)-4-(5-(2-Fluorophenyl)isoxazole-3-carbonyl)-3,6-diisobutylpiperazin-2-one (142)

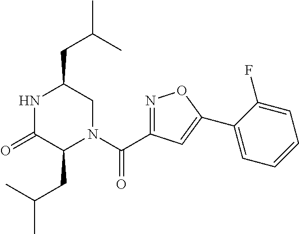

According to the method described for compound 123, 7 (99.8 mg, 0.47 mmol) was coupled with 5-(2-fluorophenyl)isoxazole-3-carboxylic acid 42 (116.8 mg, 0.564 mmol) to give the product 142 (52.6 mg, 27.9% yield) as a white solid.

¹H NMR (400 MHz, CDCl₃): δ 7.97-7.93 (1H, m), 7.48-7.43 (1H, m), 7.30-7.18 (2H, m), 7.08 (1H, d, J=3.6 Hz), 6.59 (1H, s), 5.28-5.24 (1H, m), 4.82-4.76 (1H, m), 3.78-3.65 (1H, m), 3.10 (1H, dd, J=10.8, 13.6 Hz), 1.91-1.67 (4H, m), 1.42-1.33 (2H, m), 1.08-0.90 (12H, m).

MS (ESI): m/z 402.2 [M+1]⁺.

### Example 126

### (3S,6S)-4-(5-(4-Chloro-2-fluorophenyl)isoxazole-3-carbonyl)-3,6-diisobutylpiperazin-2-one (143)

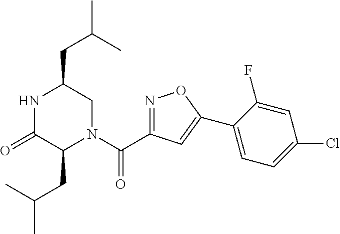

According to the method described for compound 123, 7 (99.8 mg, 0.47 mmol) was coupled with 5-(4-chloro-2-fluorophenyl)isoxazole-3-carboxylic acid 36 (136.26 mg, 0.564 mmol) to give the product 143 (93.5 mg, 45.6% yield) as a white solid.

¹H NMR (400 MHz, CDCl₃): δ 7.92-7.87 (1H, m), 7.30-7.24 (2H, m), 7.07 (1H, d, J=3.6 Hz), 6.47 (1H, s), 5.28-5.24 (1H, m), 4.79-4.75 (1H, m), 3.78-3.65 (1H, m), 3.10 (1H, dd, J=10.8, 13.6 Hz), 1.91-1.67 (4H, m), 1.42-1.33 (2H, m), 1.08-0.90 (12H, m).

MS (ESI): m/z 436.1 [M+1]⁺.

### Example 127

### (3S,6S)-4-(3-(4-Fluorophenyl)isoxazole-5-carbonyl)-3,6-diisobutylpiperazin-2-one (144)

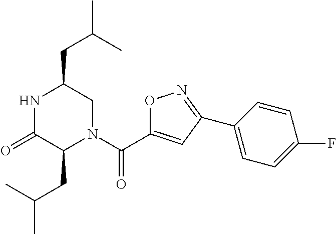

According to the method described for compound 123, 7 (99.8 mg, 0.47 mmol) was coupled with 3-(4-fluorophenyl)isoxazole-5-carboxylic acid (116.8 mg, 0.564 mmol) to give the product 144 (50.9 mg, 27% yield) as a white solid.

¹H NMR (400 MHz, CDCl₃): δ 7.83-7.79 (2H, m), 7.20-7.15 (3H, m), 6.28 (1H, s), 5.22 (1H, dd, J=4, 9.2 Hz), 4.44 (1H, dd, J=4, 14 Hz), 3.78-3.68 (1H, m), 3.17 (1H, dd, J=11.2, 14 Hz), 1.94-1.85 (1H, m), 1.83-1.68 (3H, m), 1.42-1.36 (2H, m), 1.07-0.94 (12H, m).

MS (ESI): m/z 402.3 [M+1]⁺.

### Example 128

### (3S,6S)-4-(3-(4-Fluorophenyl)isoxazole-5-carbonyl)-3-isobutyl-6-phenylpiperazin-2-one(145)

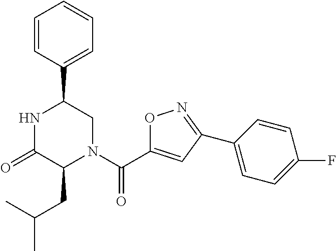

According to the method described for compound 123, 11 (100 mg, 0.43 mmol) was coupled with 3-(4-fluorophenyl)isoxazole-5-carboxylic acid (106.9 mg, 0.516 mmol) to give the product 145 (111.9 mg, 61.7% yield) as a white solid.

¹H NMR (400 MHz, CDCl₃): δ 7.84-7.80 (2H, m), 7.42-7.37 (5H, m), 7.21-7.14 (3H, m), 6.30 (1H, s), 5.31-5.28 (1H, m), 4.88-4.78 (1H, m), 4.57-4.51 (1H, m), 3.44-3.38 (1H, m), 2.04-1.72 (3H, m), 1.09 (3H, d, J=6.4 Hz), 1.00 (3H, d, J=6.4 Hz).

MS (ESI): m/z 422.2 [M+1]⁺.

### Example 129

### (3S,6S)-4-(3-(4-Fluorophenyl)isoxazole-5-carbonyl)-3-isobutyl-6-isopropylpiperazin-2-one (146)

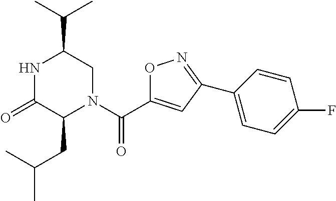

According to the method described for compound 123, 8 (40 mg, 0.2 mmol) was coupled with 3-(4-fluorophenyl)isoxazole-5-carboxylic acid (50 mg, 0.24 mmol) to give the product 146 (79 mg, 97% yield) as a white solid.

¹H NMR (400 MHz, CDCl₃): δ 7.83-7.79 (2H, m), 7.20-7.15 (3H, m), 6.24 (1H, s), 5.23-5.20 (1H, m), 4.46-4.42 (1H, m), 3.48-3.40 (1H, m), 3.30-3.20 (1H, m), 1.96-1.62 (4H, m), 1.10-0.90 (12H, m).

MS (ESI): m/z 388.1 [M+1]⁺.

### Example 130

### (3S,6R)-4-(3-(4-Fluorophenyl)isoxazole-5-carbonyl)-3-isobutyl-6-((methylthio)methyl)piperazin-2-one (147)

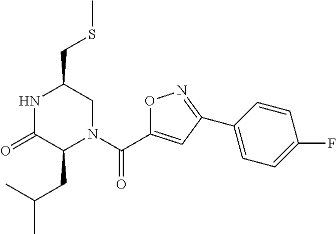

According to the method described for compound 123, 10 (43.3 mg, 0.2 mmol) was coupled with 3-(4-fluorophenyl)isoxazole-5-carboxylic acid (50 mg, 0.24 mmol) to give the product 147 (22.8 mg, 28.1% yield) as colorless gum.

¹H NMR (400 MHz, CDCl₃): δ 7.83-7.79 (2H, m), 7.20-7.15 (3H, m), 6.46 (1H, s), 5.27-5.23 (1H, m), 4.57-4.52 (1H, m), 3.84-3.73 (1H, m), 3.31-3.24 (1H, m), 2.79-2.74 (1H, m), 2.44-2.38 (1H, m), 2.14 (3H, s), 1.97-1.69 (3H, m), 1.07 (3H, d, J=6.4 Hz), 0.99 (3H, d, J=6.4 Hz).

MS (ESI): m/z 406.1 [M+1]⁺.

### Example 131

### (3S,6S)-4-(3-(4-Fluorophenyl)-1,2,4-oxadiazole-5-carbonyl)-3,6-diisobutylpiperazin-2-one(148)

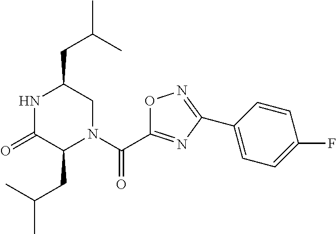

According to the method described for compound 123, 7 (84.8 mg, 0.4 mmol) was coupled with 3-(4-fluorophenyl)-1,2,4-oxadiazole-5-carboxylic acid 66 (100 mg, 0.48 mmol) to give the product 148 (21.4 mg, 13.3% yield) as a white solid.

¹H NMR (400 MHz, CDCl₃): δ 8.13-8.08 (2H, m), 7.23-7.18 (2H, m), 5.95 (1H, s), 5.28-5.21 (1H, m), 4.62-4.57 (1H, m), 3.88-3.80 (1H, m), 3.21-3.15 (1H, m), 1.96-1.64 (4H, m), 1.44-1.36 (2H, m), 1.10-0.92 (12H, m).

MS (ESI): m/z 403.1 [M+1]⁺.

### Example 132

### (3S,6S)-4-(3-(4-Fluorophenyl)-1,2,4-oxadiazole-5-carbonyl)-3-isobutyl-6-propylpiperazin-2-one (149)

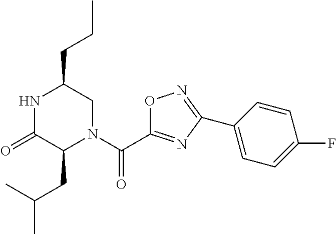

According to the method described for compound 123, 16 (100 mg, 0.504 mmol) was coupled with 3-(4-fluorophenyl)-1,2,4-oxadiazole-5-carboxylic acid 66 (123.3 mg, 0.55 mmol) to give the product 149 (3.6 mg, 1.8% yield) as a colorless oil.

¹H NMR (400 MHz, CDCl₃): δ 8.13-8.09 (2H, m), 7.23-7.17 (2H, m), 5.80 (1H, s), 5.29-5.22 (1H, m), 4.65-4.60 (1H, m), 3.78-3.65 (1H, m), 3.21 (1H, dd, J=10.8, 13.6 Hz), 1.95-1.86 (1H, m), 1.81-1.64 (2H, m), 1.59-1.40 (4H, m), 1.10-0.76 (9H, m).

MS (ESI): m/z 389.1 [M+1]⁺.

### Example 133

### (3S,6S)-3-Isobutyl-4-(3-phenyl-1,2,4-oxadiazole-5-carbonyl)-6-propylpiperazin-2-one(150)

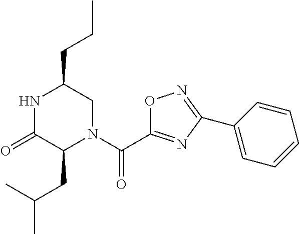

According to the method described for compound 123, 16 (100 mg, 0.504 mmol) was coupled with 3-phenyl-1,2,4-oxadiazole-5-carboxylic acid 66 (113 mg, 0.55 mmol) to give the product 150 (3.1 mg, 1.7% yield) as a colorless oil.

¹H NMR (400 MHz, CDCl₃): δ 8.13-8.09 (2H, m), 7.57-7.50 (3H, m), 5.81 (1H, s), 5.32-5.26 (1H, m), 4.72-4.65 (1H, m), 3.82-3.73 (1H, m), 3.24-3.18 (1H, m), 1.94-1.66 (3H, m), 1.60-1.37 (4H, m), 1.10-0.77 (9H, m).

MS (ESI): m/z 371.1 [M+1]⁺.

### Example 134

### (3S,6S)-4-(3-(4-Fluorophenyl)-1,2,4-oxadiazole-5-carbonyl)-3-isobutyl-6-phenylpiperazin-2-one(151)

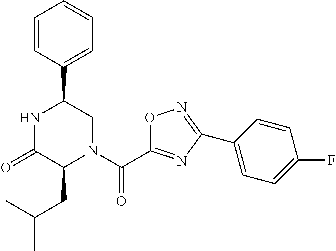

According to the method described for compound 123, 11 (100 mg, 0.43 mmol) was coupled with 3-(4-fluorophenyl)-1,2,4-oxadiazole-5-carboxylic acid 66 (107.4 mg, 0.516 mmol) to give the product 151 (45.1 mg, 24.8% yield) as a white solid.

¹H NMR (400 MHz, CDCl₃): δ 8.14-8.09 (2H, m), 7.47-7.35 (5H, m), 7.26-7.18 (2H, m), 6.15 (1H, s), 5.37-5.34 (1H, m), 4.95-4.89 (1H, m), 4.71-4.67 (1H, m), 3.46-3.39 (1H, m), 2.04-1.71 (3H, m), 1.11 (3H, d, J=6.4 Hz), 1.02 (3H, d, J=6.4 Hz).

MS (ESI): m/z 423.1 [M+1]⁺.

### Example 135

### (3S,6S)-6-Cyclopentyl-4-(3-(4-fluorophenyl)-1,2,4-oxadiazole-5-carbonyl)-3-isobutylpiperazin-2-one (152)

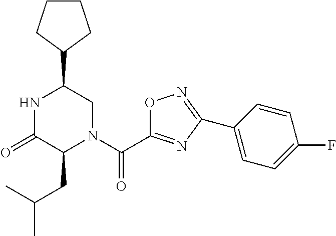

According to the method described for compound 123, 17 (64.6 mg, 0.288 mmol) was coupled with 3-(4-fluorophenyl)-1,2,4-oxadiazole-5-carboxylic acid 66 (50 mg, 0.24 mmol) to give the product 152 (42.1 mg, 42.3% yield) as a colorless oil.

¹H NMR (400 MHz, CDCl₃): δ 8.12-8.08 (2H, m), 7.23-7.17 (2H, m), 6.09 (1H, s), 5.27-5.19 (1H, m), 4.72-4.68 (1H, m), 3.59-3.53 (1H, m), 3.26-3.19 (1H, m), 1.95-1.55 (12H, m), 1.09 (3H, d, J=6.4 Hz), 0.99 (3H, d, J=6.4 Hz).

MS (ESI): m/z 415.2 [M+1]⁺.

### Example 136

### (3S,6S)-4-(3-(4-Fluorophenyl)-1,2,4-oxadiazole-5-carbonyl)-3-isobutyl-6-isopropylpiperazin-2-one (153)

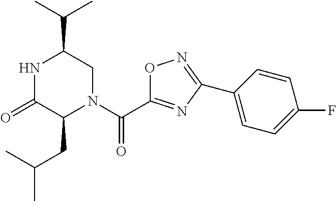

According to the method described for compound 123, 8 (79.3 mg, 0.4 mmol) was coupled with 3-(4-fluorophenyl)-1,2,4-oxadiazole-5-carboxylic acid 66 (100 mg, 0.48 mmol) to give the product 153 (27.1 mg, 17.4% yield) as a colorless oil.

¹H NMR (400 MHz, CDCl₃): δ 8.13-8.08 (2H, m), 7.23-7.17 (2H, m), 6.03 (1H, s), 5.28-5.22 (1H, m), 4.72-4.68 (1H, m), 3.61-3.56 (1H, m), 3.29-3.23 (1H, m), 1.95-1.61 (4H, m), 1.10-0.93 (12H, m).

MS (ESI): m/z 389.1 [M+1]⁺.

### Example 137

### (3S,6R)-4-(3-(4-Fluorophenyl)-1,2,4-oxadiazole-5-carbonyl)-3-isobutyl-6-((methylthio)methyl)piperazin-2-one (154)

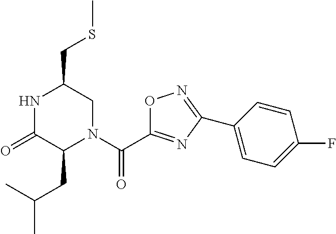

According to the method described for compound 123, 10 (86.6 mg, 0.4 mmol) was coupled with 3-(4-fluorophenyl)-1,2,4-oxadiazole-5-carboxylic acid 66 (100 mg, 0.48 mmol) to give the product 154 (23.8 mg, 14.6% yield) as a colorless oil.

¹H NMR (400 MHz, CDCl₃): δ 8.13-8.09 (2H, m), 7.23-7.17 (2H, m), 6.39 (1H, s), 5.32-5.25 (1H, m), 4.76-4.68 (1H, m), 3.92-3.84 (1H, m), 3.31-3.25 (1H, m), 2.73-2.68 (1H, m), 2.48-2.43 (1H, m), 2.14 (3H, s), 1.97-1.67 (3H, m), 1.09 (3H, d, J=6.4 Hz), 1.00 (3H, d, J=6.4 Hz).

MS (ESI): m/z 407.1 [M+1]⁺.

### Example 138

### (3S,6S)-3,6-Diisobutyl-4-[5-(4-nitro-phenyl)-isoxazole-3-carbonyl]-piperazin-2-one (155)

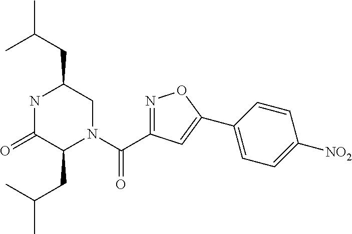

Compound 7 (60 mg, 0.28 mmol) and 5-(4-nitrophenyl)-isoxazole-3-carboxylic acid (60 mg, 0.28 mmol) were coupled according to the procedure described for compound 71 to furnish 155 (90 mg, 74% yield) as a pale yellow solid.

¹H NMR (400 MHz, CDCl₃): δ 8.37-7.34 (m, 2H), 7.99-7.96 (m, 2H), 7.12 (s, 1H), 6.20 (s, 1H), 5.29-5.25 (m, 1H), 4.88-4.76 (m, 1H), 3.79-3.69 (m, 1H), 3.18 (dd, J=14 & 10.8 Hz, 1H), 1.91-1.69 (m, 4H), 1.42-1.35 (m, 2H) and 1.08-0.92 (m, 12H).

MS (ESI): m/z 429.1 [M+1]⁺

### Example 139

### (3S,6S)-6-Cyclohexyl-4-[5-(2,4-difluoro-phenyl)-isoxazole-3-carbonyl]-3-isobutyl-piperazin-2-one (156)

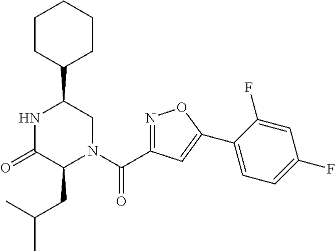

Compound 18 (48 mg, 0.20 mmol) and 5-(2,4-difluoro-phenyl)-isoxazole-3-carboxylic acid (50 mg, 0.22 mmol) were coupled according to the procedure described for compound 71 to furnish 156 (50 mg, 55.7% yield) as a white solid.

¹H NMR (400 MHz, CDCl₃): δ 7.98-7.92 (m, 1H), 7.06-6.93 (m, 3H), 6.23 (s, 1H), 5.29-5.19 (m, 1H), 4.82-4.78 (m, 1H), 3.53-3.45 (m, 1H), 3.22 (dd, J=14.0 & 11.2 Hz, 1H), 2.95 (dd, J=14.0 & 11.2 Hz, 1H), 1.87-1.69 (m, 6H), 1.50-0.91 (m, 13H).

MS (ESI): m/z 446.2 [M+1]⁺

### Example 140

### (3S,6S)-6-Cyclopentyl-4-[5-(2,4-difluoro-phenyl)-isoxazole-3-carbonyl]-3-isobutyl-piperazin-2-one (157)

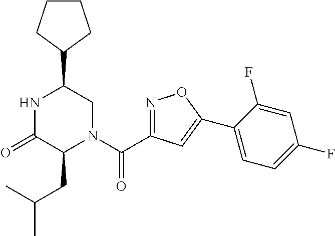

Compound 17 (45 mg, 0.2 mmol) and 5-(2,4-difluoro-phenyl)-isoxazole-3-carboxylic acid (50 mg, 0.22 mmol) were coupled according to the procedure described for compound 71 to furnish 157 (65 mg, 75.1% yield) as a white solid.

¹H NMR (400 MHz, CDCl₃): δ 7.98-7.92 (m, 1H), 7.06-6.93 (m, 3H), 6.09 (s, 1H), 5.29-5.19 (m, 1H), 4.84-4.80 (m, 1H), 3.53-3.42 (m, 1H), 3.15 (dd, J=14.0 & 11.2 Hz, 1H), 2.89 (dd, J=14.0 & 11.2 Hz, 1H), 1.89-1.52 (m, 8H), 1.32-1.22 (m, 3H), 1.08-0.76 (m, 6H).

MS (ESI): m/z 446.2 [M+1]⁺

### Example 141

### (3S,6S)-4-[5-(4-Chloro-3-fluoro-phenyl)-isoxazole-3-carbonyl]-3-isobutyl-6-propyl-piperazin-2-one (158)

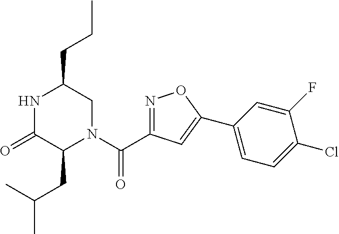

3-isobutyl-6-n-propyl-piperazin-2-one (16) (40 mg, 0.20 mmol) and [5-(3-fluoro-4-chloro)phenyl]-isoxazole-3-carboxylic acid (50 mg, 0.21 mmol) were coupled according to the procedure described for compound 71 to furnish 158 (73 mg, 85.8% yield) as a white solid.

¹H NMR (400 MHz, CDCl₃): δ 7.59-7.51 (m, 3H), 6.94 (s, 1H), 6.21 (s, 1H), 5.27 (dd J=9.6 & 4.4 Hz, 1H), 4.83 (dd, J=13.6 & 4.4 Hz, 1H), 4.78 (dd, J=13.6 & 4 Hz, 1H), 3.71-3.58 (m, 1H), 3.13 (dd, J=14.0 & 11.2 Hz, 1H), 1.91-1.66 (m, 2H), 1.54-1.38 (m, 4H) and 1.07-0.77 (m, 9H).

MS (ESI): m/z 422.1 [M+1]⁺

### Example 142

### (3S,6S)-4-[5-(4-Chloro-3-fluoro-phenyl)-isoxazole-3-carbonyl]-6-cyclohexyl-3-isobutyl-piperazin-2-one (159)

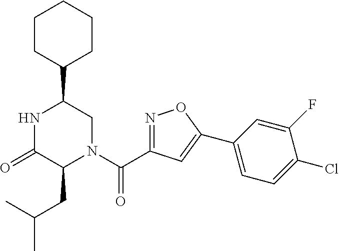

3-isobutyl-6-cyclohexyl-piperazin-2-one (18) substrate (48 mg, 0.20 mmol) and [5-(3-fluoro-4-chloro)phenyl]-isoxazole-3-carboxylic acid 51 (50 mg, 0.21 mmol) were coupled according to the procedure described for compound 71 to furnish 159 (30 mg, 32.2% yield) as a white solid.

¹H NMR (400 MHz, CDCl₃): δ 7.59-7.51 (m, 3H), 6.94 (s, 1H), 6.05 (s, 1H), 5.29-5.24 (m, 1H), 4.89 (dd, J=14.0 & 4.4 Hz, 1H), 4.77 (dd, J=13.6 & 4 Hz, 1H), 3.53-3.44 (m, 1H), 3.24 (dd, J=14.0 & 11.2 Hz, 1H), 2.96 (d, J=14.0 & 11.2 Hz, 1H), 1.87-1.63 (m, 8H), 1.54-1.01 (m, 8H) and 0.98-0.77 (m, 4H).

MS (ESI): m/z 462.1 [M+1]⁺

### Example 143

### (3S,6S)-4-[5-(4-Chloro-3-fluoro-phenyl)-isoxazole-3-carbonyl]-6-cyclopentyl-3-isobutyl-piperazin-2-one (160)

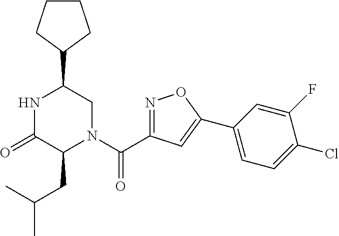

3-isobutyl-6-cyclopentyl-piperazin-2-one (17) substrate (48 mg, 0.20 mmol) and [5-(3-fluoro-4-chloro)phenyl]-isoxazole-3-carboxylic acid 51 (45 mg, 0.21 mmol) were coupled according to the procedure described for compound 71 to furnish 160 (83 mg, 92.4% yield) as a white solid.

¹H NMR (400 MHz, CDCl₃): δ 7.58-7.51 (m, 3H), 6.94 (s, 1H), 6.16 (s, 1H), 5.25 (dd, J=10.0 & 4 Hz, 1H), 4.88 (dd, J=13.6 & 4.0 Hz, 1H), 4.78 (dd, J=13.6 & 4 Hz, 1H), 3.52-3.41 (m, 1H), 3.15 (dd, J=14.0 & 11.2 Hz, 1H), 2.89 (d, J=14.0 & 11.2 Hz, 1H), 1.88-1.57 (m, 8H), 1.36-1.22 (m, 2H) and 1.07-0.77 (m, 6H).

MS (ESI): m/z 448.2 [M+1]⁺

### Example 144

### (3S,6S)-4-(5-(4-fluorophenyl)isoxazole-3-carbonyl)-3-isobutyl-6-propylpiperazin-2-one (161)

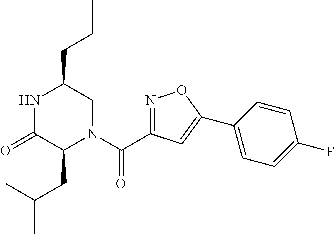

(3S,6S)-3-isobutyl-6-propylpiperazin-2-one (17) (50 mg, 0.25 mmol) and 5-(4-fluorophenyl)isoxazole-3-carboxylic acid (53 mg, 0.25 mmol) were coupled according to the procedure described for compound 71 to furnish 161 (93 mg, 95% yield) as white solid.

¹H NMR (400 MHz, CDCl₃): δ 7.80-7.77 (m, 2H), 7.21-7.17 (m, 2H), 6.86 (s, 1H), 6.08 (s, 1H), 5.29-5.28 (m, 1H), 4.89-4.78 (m, 1H), 3.71-3.63 (m, 1H), 3.17-2.83 (m, 1H), 1.92-1.65 (m, 3H), 1.58-1.37 (m, 4H), 1.08-0.77 (m, 9H);

MS (ESI): m/z 388.1 (M+1)⁺

### Example 145

### (3S,6S)-6-cyclohexyl-4-(5-(4-fluorophenyl)isoxazole-3-carbonyl)-3-isobutylpiperazin-2-one (162)

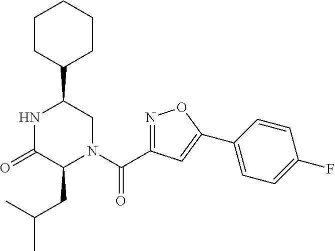

(3S,6S)-6-cyclohexyl-3-isobutylpiperazin-2-one 18 (50 mg, 0.24 mmol) and 5-(4-fluorophenyl)isoxazole-3-carboxylic acid (43 mg, 0.21 mmol) were coupled according to the procedure described for compound 71 to furnish 162 (46 mg, 51% yield) as a white solid.

¹H NMR (400 MHz, CDCl₃): δ 7.81-7.76 (m, 2H), 7.21-7.16 (m, 2H), 6.86 (s, 1H), 5.86 (s, 1H), 5.30-5.27 (m, 1H), 4.92-4.78 (m, 1H), 3.53-3.44 (m, 1H), 3.26-2.93 (m, 1H), 1.88-1.70 (m, 8H), 1.45-1.37 (m, 1H), 1.29-1.11 (m, 5H), 1.09-0.77 (m, 6H);

MS (ESI): m/z 428.1 (M+1)⁺

### Example 146

### (3S,6S)-6-cyclopentyl-4-(5-(4-fluorophenyl)isoxazole-3-carbonyl)-3-isobutyl-piperazin-2-one (163)

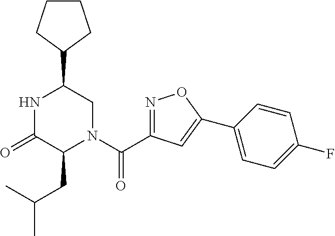

(3S,6S)-6-cyclopentyl-3-isobutylpiperazin-2-one 17 (50 mg, 0.22 mmol) and 5-(4-fluorophenyl)isoxazole-3-carboxylic acid (46 mg, 0.22 mmol) were coupled according to the procedure described for compound 71 to furnish 163 (56 mg, 61% yield) as a white solid.

¹H NMR (400 MHz, CDCl₃): δ 7.81-7.76 (m, 2H), 7.21-7.16 (m, 2H), 6.87 (s, 1H), 5.94 (s, 1H), 5.29-5.26 (m, 1H), 4.93-4.79 (m, 1H), 3.53-3.41 (m, 1H), 3.19-2.86 (m, 1H), 1.91-1.58 (m, 10H), 1.32-1.23 (m, 2H), 1.09-0.77 (m, 6H);

MS (ESI): m/z 414.1 (M+1)⁺

### Example 147

### (3S,6S)-4-(5-(2,4-difluorophenyl)isoxazole-3-carbonyl)-3,6-diisobutylpiperazin-2-one (164)

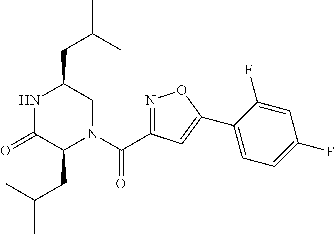

(3S,6S)-3,6-diisobutylpiperazin-2-one 7 (50 mg, 0.24 mmol) and 5-(2,4-difluorophenyl)isoxazole-3-carboxylic acid (53 mg, 0.24 mmol) were coupled according to the procedure described for compound 71 to furnish 164 (72 mg, 73% yield) as a white solid.

¹H NMR (400 MHz, CDCl₃): δ 8.00-7.93 (m, 1H), 7.07-6.95 (m, 3H), 5.88 (s, 1H), 5.30-5.22 (m, 1H), 4.81-4.76 (m, 1H), 3.78-3.70 (m, 1H), 3.15-2.79 (m, 1H), 1.89-1.61 (m, 4H), 1.42-1.31 (m, 2H) 1.09-0.76 (m, 12H);

MS (ESI): m/z 420.1 (M+1)⁺

### Example 148

### (3S,6S)-4-(5-(2,4-difluorophenyl)isoxazole-3-carbonyl)-3-isobutyl-6-propylpiperazin-2-one (165)

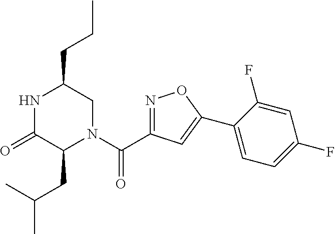

(3S,6S)-3-isobutyl-6-propylpiperazin-2-one 16 (50 mg, 0.25 mmol) and 5-(2,4-difluorophenyl)isoxazole-3-carboxylic acid (56 mg, 0.25 mmol) were coupled according to the procedure described for compound 71 to furnish 165 (93 mg, 91% yield) as white solid.

¹H NMR (400 MHz, CDCl₃): δ 7.99-7.92 (m, 1H), 7.07-6.94 (m, 3H), 5.91 (s, 1H), 5.30-5.22 (m, 1H), 4.83-4.77 (m, 1H), 3.71-3.64 (m, 1H), 3.17-2.83 (m, 1H), 1.89-1.69 (m, 3H), 1.55-1.34 (m, 4H) 1.09-0.76 (m, 9H);

MS (ESI): m/z 406.1 (M+1)⁺

### Example 149

### (3S,6S)-4-(5-(4-chlorophenyl)isoxazole-3-carbonyl)-6-(cyclopropylmethyl)-3-isobutylpiperazin-2-one (166)

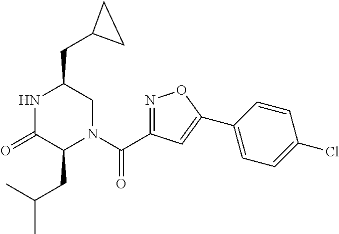

(3S,6S)-6-(cyclopropylmethyl)-3-isobutylpiperazin-2-one 13 (50 mg, 0.24 mmol) and 5-(4-chlorophenyl)isoxazole-3-carboxylic acid (53 mg, 0.24 mmol) were coupled according to the procedure described for compound 71 to furnish 166 (53 mg, 54% yield) as white solid.

¹H NMR (400 MHz, CDCl₃): δ 7.75-7.71 (m, 2H), 7.49-7.45 (m, 2H), 6.92 (s, 1H), 6.16 (s, 1H), 5.32-5.27 (m, 1H), 4.94-4.82 (m, 1H), 3.82-3.72 (m, 1H), 3.22-2.88 (m, 1H), 1.92-1.58 (m, 4H), 1.55-1.22 (m, 1H), 1.08-0.76 (m, 6H), 0.73-0.52 (m, 3H), 0.21-0.08 (m, 2H).

MS (ESI): m/z 416.1 (M+1)⁺

### Example 150

### (3S,6S)-4-(5-(4-fluorophenyl)isoxazole-3-carbonyl)-6-(cyclopropylmethyl)-3-isobutylpiperazin-2-one (167)

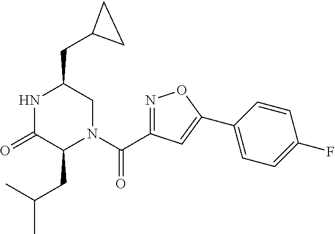

(3S,6S)-6-(cyclopropylmethyl)-3-isobutylpiperazin-2-one 13 (50 mg, 0.24 mmol) and 5-(4-fluorophenyl)isoxazole-3-carboxylic acid (53 mg, 0.24 mmol) were coupled according to the procedure described for compound 71 to furnish 167 (53 mg, 54% yield) as white solid.

¹H NMR (400 MHz, CDCl₃): δ 7.79-7.61 (m, 2H), 7.19-7.15 (m, 2H), 6.86 (s, 1H), 6.47 (s, 1H), 5.29-5.25 (m, 1H), 4.93-4.88 (m, 1H), 3.78-3.74 (m, 1H), 3.21 (dd, J=14 & 11 hz, 1H), 1.88-1.52 (m, 4H), 1.30-1.22 (m, 1H), 1.08-0.96 (m, 6H), 0.74-0.52 (m, 3H), 0.21-0.08 (m, 2H).

MS (ESI): m/z 400.1 (M+1)⁺

### Example 151

### (3S,6S)-6-(cyclopropylmethyl)-3-isobutyl-4-(5-(thiophen-2-yl)isoxazole-3-carbonyl)piperazin-2-one (168)

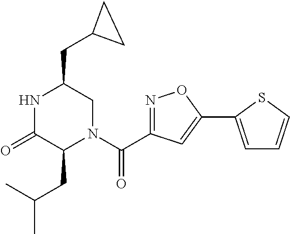

(3S,6S)-6-(cyclopropylmethyl)-3-isobutylpiperazin-2-one 13 (50 mg, 0.24 mmol) and 5-(thiophen-2-yl)isoxazole-3-carboxylic acid (46 mg, 0.24 mmol) were coupled according to the procedure described for compound 71 to furnish 168 (69 mg, 75% yield) as a brown solid.

¹H NMR (400 MHz, CDCl₃): δ 7.56-7.54 (m, 1H), 7.51-7.49 (m, 1H), 7.16-7.14 (m, 1H) 6.77 (s, 1H), 6.09 (s, 1H), 5.30-5.27 (m, 1H), 4.91-4.85 (m, 1H), 3.80-3.74 (m, 1H), 3.21-2.80 (m, 1H), 1.89-1.70 (m, 4H), 1.64-1.55 (m, 1H), 1.28-1.18 (m, 1H), 1.08-0.78 (m, 6H), 0.71-0.52 (m, 3H), 0.20-0.09 (m, 2H).

MS (ESI): m/z 388.1 (M+1)⁺.

### Example 152

### (3S,6S)-3-isobutyl-6-propyl-4-(5-(thiophen-2-yl)isoxazole-3-carbonyl)piperazin-2-one (169)

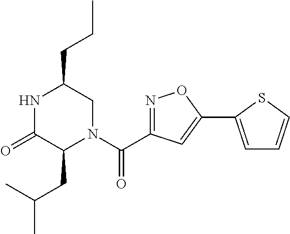

(3S,6S)-3-isobutyl-6-propylpiperazin-2-one 16 (50 mg, 0.25 mmol) 5-(thiophen-2-yl)isoxazole-3-carboxylic acid (49 mg, 0.25 mmol) were coupled according to the procedure described for compound 71 to furnish 169 (72 mg, 73% yield) as a brown solid.

¹H NMR (400 MHz, CDCl₃): δ 7.75-7.49 (m, 2H), 7.16-7.13 (m, 2H), 6.77 (s, 1H), 5.92 (s, 1H), 5.29-5.26 (m, 1H), 4.87-4.77 (m, 1H), 3.70-3.63 (m, 1H), 3.17-2.8 (m, 1H), 1.89-1.69 (m, 3H), 1.53-1.39 (m, 4H), 1.08-0.78 (m, 9H).

MS (ESI): m/z 376.1 (M+1)⁺

### Example 153

### (3S,6S)-4-(5-(4-chlorophenyl)isoxazole-3-carbonyl)-3-isobutyl-6-propylpiperazin-2-one (170)

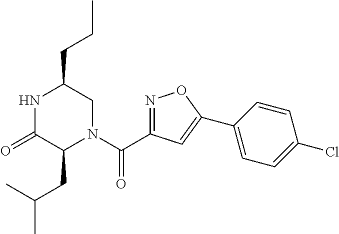

(3S,6S)-3-isobutyl-6-propylpiperazin-2-one 16 (50 mg, 0.25 mmol) and 5-(4-chlorophenyl)isoxazole-3-carboxylic acid (56 mg, 0.25 mmol) were coupled according to the procedure described for compound 71 to furnish 170 (53 mg, 52% yield) as white solid.

¹H NMR (400 MHz, CDCl₃): δ 7.74-7.71 (m, 2H), 7.49-7.46 (m, 2H), 6.91 (s, 1H), 5.89 (s, 1H), 5.30-5.27 (m, 1H), 4.89-4.78 (m, 1H), 3.71-3.63 (m, 1H), 3.17-2.83 (m, 1H), 1.89-1.71 (m, 3H), 1.53-1.39 (m, 4H), 1.08-0.77 (m, 9H);

MS (ESI): m/z 404.1 (M+1)⁺

### Example 154

### (3S,6S)-4-(5-(4-chlorophenyl)isoxazole-3-carbonyl)-6-cyclohexyl-3-isobutylpiperazin-2-one (171)

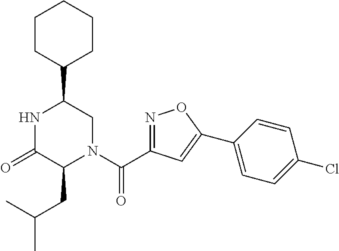

(3S,6S)-6-cyclohexyl-3-isobutylpiperazin-2-one 18 (50 mg, 0.21 mmol) and 5-(4-chlorophenyl)isoxazole-3-carboxylic acid (47 mg, 0.21 mmol) were coupled according to the procedure described for compound 71 to furnish 171 (71 mg, 76% yield) as white solid.

¹H NMR (400 MHz, CDCl₃): δ 7.73-7.71 (m, 2H), 7.48-7.45 (m, 2H), 6.91 (s, 1H), 6.00 (s, 1H), 5.29-5.26 (m, 1H), 4.91-4.77 (m, 1H), 3.53-3.44 (m, 1H), 3.26-2.92 (m, 1H), 1.88-1.68 (m, 9H), 1.42-1.39 (m, 1H), 1.28-1.11 (m, 4H), 1.08-0.77 (m, 6H).

MS (ESI): m/z 444.2 (M+1)⁺

### Example 155

### (3S,6S)-6-cyclohexyl-3-isobutyl-4-(5-(thiophen-2-yl)isoxazole-3-carbonyl)piperazin-2-one (172)

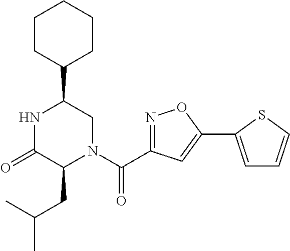

(3S,6S)-6-cyclohexyl-3-isobutylpiperazin-2-one 18 (50 mg, 0.21 mmol) and 5-(thiophen-2-yl)isoxazole-3-carboxylic acid (41 mg, 0.21 mmol) were coupled according to the procedure described for compound 71 to furnish 172 (70 mg, 80% yield) as a brown solid.

¹H NMR (400 MHz, CDCl₃): δ 7.557.48 (m, 2H), 7.16-7.13 (m, 2H), 6.77 (s, 1H), 5.96 (s, 1H), 5.29-5.24 (m, 1H), 4.89-4.76 (m, 1H), 3.52-3.43 (m, 1H), 3.26-2.94 (m, 1H), 1.87-1.69 (m, 9H), 1.44-1.39 (m, 1H), 1.28-1.11 (m, 4H), 1.08-0.78 (m, 6H);

MS (ESI): m/z 416.1 (M+1)⁺

### Example 156

### (3S,6S)-4-(5-(4-chlorophenyl)isoxazole-3-carbonyl)-6-cyclopentyl-3-isobutylpiperazin-2-one (173)

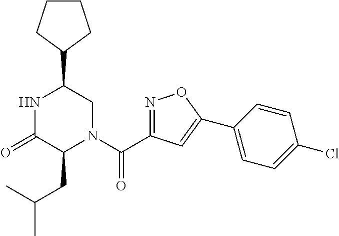

(3S,6S)-6-cyclopentyl-3-isobutylpiperazin-2-one 17 (50 mg, 0.21 mmol) and 5-(4-chlorophenyl)isoxazole-3-carboxylic acid (50 mg, 0.22 mmol) were coupled according to the procedure described for compound 71 to furnish 173 (57 mg, 59% yield) as a white solid.

¹H NMR (400 MHz, CDCl₃): δ 7.75-7.70 (m, 2H), 7.51-7.45 (m, 2H), 6.89 (s, 1H), 6.16 (s, 1H), 5.28-5.24 (m, 1H), 4.91-4.78 (m, 1H), 3.52-3.41 (m, 1H), 3.18-2.80 (m, 1H), 1.88-1.57 (m, 10H), 1.31-1.27 (m, 2H), 1.07-0.76 (m, 6H);

MS (ESI): m/z 430.2 (M+1)⁺

### Example 157

### (3S,6S)-6-cyclopentyl-3-isobutyl-4-(5-(thiophen-2-yl)isoxazole-3-carbonyl)piperazin-2-one (174)

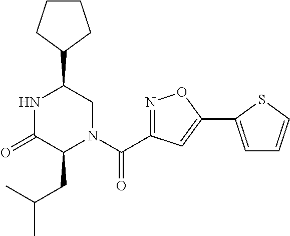

(3S,6S)-6-cyclopentyl-3-isobutylpiperazin-2-one 17 (50 mg, 0.22 mmol) and 5-(thiophen-2-yl)isoxazole-3-carboxylic acid (44 mg, 0.22 mmol) were coupled according to the procedure described for compound 71 to furnish 174 (47 mg, 52% yield) as a brown solid.

¹H NMR (400 MHz, CDCl₃): δ 7.53-7.52 (m, 1H), 7.49-7.47 (m, 1H), 7.14-7.11 (m, 1H), 6.76 (s, 1H), 5.19 (s, 1H), 5.27-5.22 (m, 1H), 4.88-4.77 (m, 1H), 3.51-3.42 (m, 1H), 3.16-2.83 (m, 1H), 1.89-1.56 (m, 10H), 1.32-1.24 (m, 2H), 1.08-0.77 (m, 6H);

MS (ESI): m/z 402.1 (M+1)⁺

### Example 158

### (3S,6S)-4-(5-(4-fluorophenyl)isoxazole-3-carbonyl)-3-isobutyl-6-isopropylpiperazin-2-one (175)

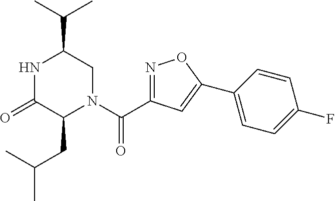

(3S,6S)-3-isobutyl-6-isopropylpiperazin-2-one 8 (50 mg, 0.25 mmol) and 5-(4-fluorophenyl)isoxazole-3-carboxylic acid (52 mg, 0.25 mmol) were coupled according to the procedure described for compound 71 to furnish 175 (43 mg, 44% yield) as white solid.

¹H NMR (400 MHz, CDCl₃): δ 7.80-7.76 (m, 2H), 7.21-7.16 (m, 2H), 6.87 (s, 1H), 5.97 (s, 1H), 5.31-5.27 (m, 1H), 4.92-4.77 (m, 1H), 3.53-3.44 (m, 1H), 3.23-2.90 (m, 1H), 1.90-1.69 (m, 4H), 1.08-0.77 (m, 12H).

MS (ESI): m/z 388.1 (M+1)⁺

### Example 159

### (3S,6S)-3,6-diisobutyl-4-(5-(4-(methylsulfonyl)phenyl)isoxazole-3-carbonyl)piperazin-2-one (176)

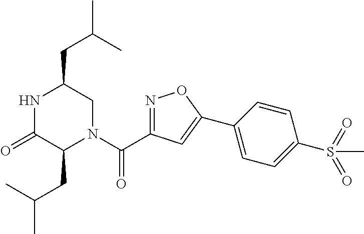

(3S,6S)-3,6-diisobutylpiperazin-2-one 7 (50 mg, 0.24 mmol) and 5-(4-(methylsulfonyl)phenyl)isoxazole-3-carboxylic acid (63 mg, 0.24 mmol) were coupled according to the procedure described for compound 71 to furnish 176 (73 mg, 67% yield) as a white solid.

¹H NMR (400 MHz, CDCl₃): δ 8.09 (d, 2H, J=8 Hz), 8.01-7.99 (d, 2H, J=8

Hz), 7.1 (s, 1H), 6.13 (s, 1H), 5.29-5.26 (m, 1H), 4.88-4.77 (m, 1H), 3.79-3.69 (m, 1H), 3.17-2.88 (m, 1H), 3.10 (s, 3H), 1.90-1.85 (m, 1H), 1.80-1.67 (m, 3H), 1.43-1.36 (m, 2H), 1.08-0.78 (m, 12H);

MS (ESI): m/z 462.2 (M+1)⁺

### Example 160

### (3S,6S)-4-(5-(4-chloro-3-fluorophenyl)isoxazole-3-carbonyl)-3,6-diisobutylpiperazin-2-one (177)

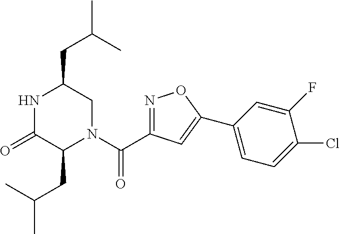

(3S,6S)-3,6-diisobutylpiperazin-2-one 7 (50 mg, 0.24 mmol) and 5-(4-chloro-3-fluorophenyl)isoxazole-3-carboxylic acid 51 (59 mg, 0.24 mmol) were coupled according to the procedure described for compound 71 to furnish 177 (78 mg, 76% yield) as white solid.

¹H NMR (400 MHz, CDCl₃): δ 7.59-7.50 (m, 3H), 7.21-7.16 (m, 2H), 6.94 (s, 1H), 5.94 (s, 1H), 5.29-5.26 (m, 1H), 4.87-4.76 (m, 1H), 3.77-3.69 (m, 1H), 3.15-2.82 (m, 1H), 1.92-1.83 (m, 1H), 1.79-1.65 (m, 3H), 1.42-1.30 (m, 2H), 1.08-0.77 (m, 12H);

MS (ESI): m/z 436.1 (M+1)⁺

### Example 161

### (3S,6S)-4-(5-(4-fluorophenyl)isoxazole-3-carbonyl)-3-isobutyl-6-phenylpiperazin-2-one (178)

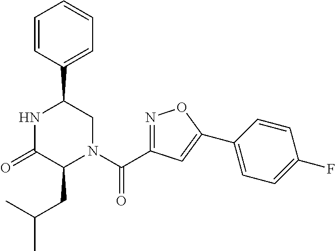

(3S,6S)-3-isobutyl-6-phenylpiperazin-2-one 11 (50 mg, 0.22 mmol) and 5-(4-fluorophenyl)isoxazole-3-carboxylic acid (45 mg, 0.22 mmol) were coupled according to the procedure described for compound 71 to furnish 178 (69 mg, 76% yield) as white solid.

¹H NMR (400 MHz, CDCl₃): δ 7.82-7.78 (m, 2H), 7.44-7.36 (m, 5H), 7.22-7.16 (m, 2H), 6.89 (s, 1H), 6.04 (s, 1H), 5.43-5.36 (m, 1H), 4.97-4.88 (m, 2H), 3.37-3.06 (m, 1H), 2.04-1.94 (m, 1H), 1.93-1.84 (m, 1H), 1.80-1.70 (m, 1H), 1.11-0.81 (m, 6H).

MS (ESI): m/z 422.1 (M+1)⁺.

### Example 162

### (3S,6S)-3,6-diisobutyl-4-(5-(4-(trifluoromethoxy)phenyl)isoxazole-3-carbonyl)piperazin-2-one (179)

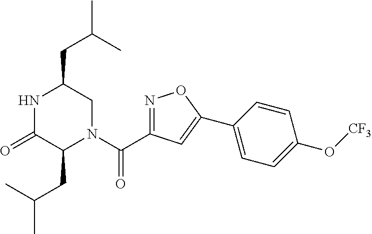

(3S,6S)-3,6-diisobutylpiperazin-2-one 7 (50 mg, 0.24 mmol) and 5-(4-(trifluoromethoxy)phenyl)isoxazole-3-carboxylic acid 47 (64 mg, 0.24 mmol) were coupled according to the procedure described for compound 71 to furnish 179 (57 mg, 52% yield) as white solid.

¹H NMR (400 MHz, CDCl₃): δ 7.84 (d, 2H, J=5.2 Hz), 7.35 (d, 2H, J=5.0 Hz), 6.93 (s, 1H), 6.92 (s, 1H), 5.95 (s, 1H), 5.30-5.26 (m, 1H), 4.89-4.85 (m, 1H), 3.77-3.72 (m, 1H), 3.15-2.81 (m, 1H), 1.89-1.67 (m, 4H), 1.42-1.33 (m, 2H), 1.08-0.77 (m, 12H);

MS (ESI): m/z 468.1 (M+1)⁺.

### Example 163

### (3S,6S)-6-cyclopropyl-4-(5-(4-fluorophenyl)isoxazole-3-carbonyl)-3-isobutylpiperazin-2-one (180)

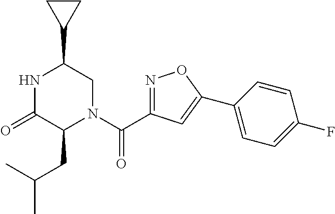

(3S,6S)-6-cyclopropyl-3-isobutylpiperazin-2-one 12 (50 mg, 0.25 mmol) and 5-(4-fluorophenyl)isoxazole-3-carboxylic acid (53 mg, 0.25 mmol) were coupled according to the procedure described for compound 71 to furnish 180 (78 mg, 79% yield) as white solid.

¹H NMR (400 MHz, CDCl₃): δ 7.80-7.77 (m, 2H), 7.20-7.16 (m, 2H), 6.85 (s, 1H), 6.05 (s, 1H), 5.29-5.24 (m, 1H), 4.94-4.86 (m, 1H), 3.35-3.02 (m, 1H), 2.88-2.77 (m, 1H), 1.92-1.88 (m, 1H), 1.83-1.71 (m, 2H), 1.09-0.92 (m, 6H), 0.79-0.75 (m, 2H), 0.64-0.60 (m, 2H), 0.43-0.27 (m, 1H).

MS (ESI): m/z 386.1 (M+1)⁺.

### Example 164

### (3S,6S)-4-(5-(4-chlorophenyl)isoxazole-3-carbonyl)-3-isobutyl-6-isopropylpiperazin-2-one (181)

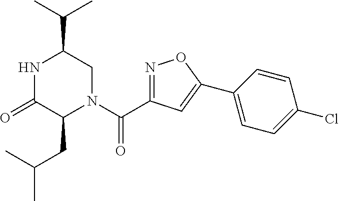

(3S,6S)-3-isobutyl-6-isopropylpiperazin-2-one 8 (50 mg, 0.25 mmol) and 5-(4-chlorophenyl)isoxazole-3-carboxylic acid (56 mg, 0.25 mmol) were coupled according to the procedure described for compound 71 to furnish 181 (83 mg, 82% yield) as white solid.

¹H NMR (400 MHz, CDCl₃): δ 7.74-7.71 (m, 2H), 7.49-7.45 (m, 2H), 6.91 (s, 1H), 6.01 (s, 1H) 5.30-5.26 (m, 1H) 4.91-4.77 (m, 1H), 3.53-3.45 (m, 1H), 3.23-2.89 (m, 1H), 1.91-1.83 (m, 1H), 1.77-1.68 (m, 3H), 1.08-0.77 (m, 12H).

MS (ESI): m/z 404.1 (M+1)⁺.

### Example 165

### (3S,6R)-4-(5-(4-chlorophenyl)isoxazole-3-carbonyl)-3-isobutyl-6-((methylthio)methyl)piperazin-2-one (182)

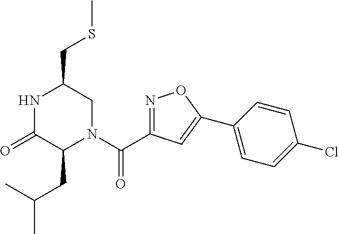

(3S,6R)-3-isobutyl-6-((methylthio)methyl)piperazin-2-one 10 (50 mg, 0.23 mmol) and 5-(4-chlorophenyl)isoxazole-3-carboxylic acid (52 mg, 0.23 mmol) were coupled according to the procedure described for compound 71 to furnish 182 (50 mg, 51% yield) as white solid.

¹H NMR (400 MHz, CDCl₃): δ 7.75-7.71 (m, 2H), 7.49-7.46 (m, 2H), 6.92 (s, 1H), 6.45 (s, 1H) 5.34-5.28 (m, 1H) 4.96-4.82 (m, 1H), 3.84-3.74 (m, 1H), 3.24-2.89 (m, 1H), 2.82-2.72 (m, 1H), 2.44-2.36 (m, 1H), 2.14 (s, 3H), 1.70-1.80 (m, 1H), 1.79-1.71 (m, 2H), 1.09-0.77 (m, 6H);

MS (ESI): m/z 422.1 (M+1)⁺.

### Example 166

### (3S,6S)-4-(5-(4-chlorophenyl)isoxazole-3-carbonyl)-3-isobutyl-6-phenylpiperazin-2-one (183)

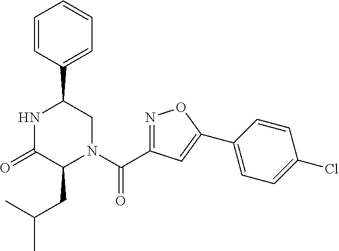

(3S,6S)-3-isobutyl-6-phenylpiperazin-2-one 11 (50 mg, 0.22 mmol) and 5-(4-chlorophenyl)isoxazole-3-carboxylic acid (48 mg, 0.22 mmol) were coupled according to the procedure described for compound 71 to furnish 183 (85 mg, 90% yield) as white solid.

¹H NMR (400 MHz, CDCl₃): δ 7.75-7.72 (m, 2H), 7.49-7.43 (m, 2H), 7.42-7.35 (m, 5H), 6.93 (s, 1H) 6.09 (s, 1H) 5.39-5.35 (m, 1H) 4.97-4.87 (m, 2H), 3.37-3.31 (m, 1H), 2.01-1.92 (m, 1H), 1.90-1.83 (m, 1H), 1.78-1.71 (m, 1H), 1.11-0.81 (m, 6H).

MS (ESI): m/z 438.1 (M+1)⁺

### Example 167

### (3S,6S)-4-(5-(3,4-difluorophenyl)isoxazole-3-carbonyl)-3-isobutyl-6-propylpiperazin-2-one (184)

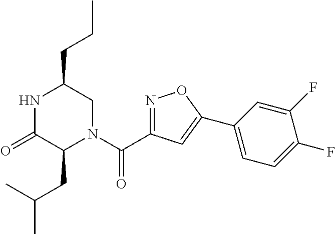

(3S,6S)-3-isobutyl-6-propylpiperazin-2-one 16 (50 mg, 0.22 mmol) and 5-(3,4-difluorophenyl)isoxazole-3-carboxylic acid 35 (48 mg, 0.22 mmol) were coupled according to the procedure described for compound 71 to furnish 184 (85 mg, 90% yield) as white solid.

¹H NMR (400 MHz, CDCl₃): δ 7.65-7.60 (m, 1H), 7.56-7.52 (m, 1H), 7.33-7.25 (m, 1H) 6.89 (s, 1H), 6.03 (s, 1H), 5.29-5.26 (m, 1H), 4.88-4.77 (m, 1H), 3.70-3.63 (m, 1H), 3.17-2.84 (m, 1H), 1.92-1.81 (m, 1H), 1.79-1.69 (m, 2H), 1.55-1.37 (m, 4H), 1.08-0.77 (m, 6H).

MS (ESI): m/z 406.1 (M+1)⁺.

### Example 168

### (3S,6S)-6-cyclohexyl-4-(5-(3,4-difluorophenyl)isoxazole-3-carbonyl)-3-isobutylpiperazin-2-one (185)

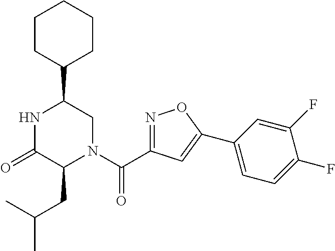

(3S,6S)-6-cyclohexyl-3-isobutylpiperazin-2-one 18 (50 mg, 0.21 mmol) and 5-(3,4-difluorophenyl)isoxazole-3-carboxylic acid 35 (48 mg, 0.21 mmol) were coupled according to the procedure described for compound 71 to furnish 185 (39 mg, 41% yield) as white solid.

¹H NMR (400 MHz, CDCl₃): δ 7.65-7.60 (m, 1H), 7.56-7.52 (m, 1H), 7.33-7.24 (m, 1H) 6.89 (s, 1H), 5.87 (s, 1H), 5.29-5.25 (m, 1H), 4.91-4.77 (m, 1H), 3.53-3.42 (m, 1H), 3.27-2.93 (m, 1H), 1.89-1.70 (m, 8H), 1.45-1.39 (m, 2H), 1.29-1.12 (m, 4H), 1.08-0.78 (m, 6H).

MS (ESI): m/z 446.1 (M⁺+1).

### Example 169

### (3S,6S)-6-cyclopentyl-4-(5-(3,4-difluorophenyl)isoxazole-3-carbonyl)-3-isobutylpiperazin-2-one (186)

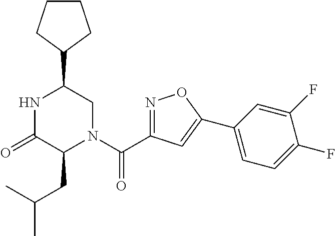

(3S,6S)-6-cyclopentyl-3-isobutylpiperazin-2-one 17 (50 mg, 0.22 mmol) and 5-(3,4-difluorophenyl)isoxazole-3-carboxylic acid 35 (50 mg, 0.22 mmol) were coupled according to the procedure described for compound 71 to furnish 186 (87 mg, 90% yield) as a white solid.

¹H NMR (400 MHz, CDCl₃): δ 7.65-7.60 (m, 1H), 7.56-7.52 (m, 1H), 7.33-7.25 (m, 1H) 6.89 (s, 1H), 5.92 (s, 1H), 5.29-5.25 (m, 1H), 4.92-4.87 (m, 1H), 3.52-3.47 (m, 1H), 3.19-2.89 (m, 1H), 1.92-1.59 (m, 10H), 1.31-1.24 (m, 2H), 1.08-0.77 (m, 6H).

MS (ESI): m/z 432.1 (M+1)⁺

### Example 170

### (3S,6S)-4-(5-(3,4-difluorophenyl)isoxazole-3-carbonyl)-3-isobutyl-6-isopropylpiperazin-2-one (187)

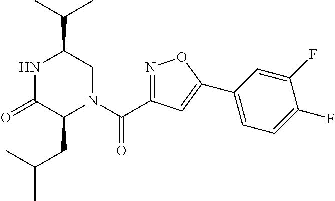

(3S,6S)-3-isobutyl-6-isopropylpiperazin-2-one 8 (50 mg, 0.25 mmol) and 5-(3,4-difluorophenyl)isoxazole-3-carboxylic acid (57 mg, 0.25 mmol) were coupled according to the procedure described for compound 71 to furnish 187 (79 mg, 77% yield) as a white solid.

¹H NMR (400 MHz, CDCl₃): δ 7.65-7.60 (m, 1H), 7.56-7.52 (m, 1H), 7.33-7.26 (m, 1H) 6.90 (s, 1H), 5.97 (s, 1H), 5.29-5.26 (m, 1H), 4.90-4.77 (m, 1H), 3.53-3.43 (m, 1H), 3.24-2.90 (m, 1H), 1.91-1.84 (m, 1H), 1.79-1.68 (m, 3H), 1.08-0.78 (m, 6H).

MS (ESI): m/z 406.1 (M+1)⁺.

### Example 171

### (3S,6S)-4-(5-(3,4-difluorophenyl)isoxazole-3-carbonyl)-3-isobutyl-6-phenylpiperazin-2-one (188)

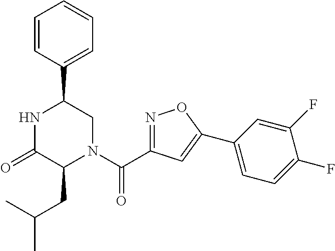

(3S,6S)-3-isobutyl-6-phenylpiperazin-2-one 11 (50 mg, 0.22 mmol) and 5-(3,4-difluorophenyl)isoxazole-3-carboxylic acid 35 (49 mg, 0.22 mmol) were coupled according to the procedure described for compound 71 to furnish 188 (81 mg, 86% yield) as a white solid.

¹H NMR (400 MHz, CDCl₃): δ 7.66-7.61 (m, 1H), 7.58-7.53 (m, 1H), 7.45-7.36 (m, 5H), 7.34-7.27 (m, 1H) 6.92 (s, 1H), 6.02 (s, 1H), 5.39-5.35 (m, 1H), 4.96-4.76 (m, 2H), 3.38-3.07 (m, 1H), 2.01-1.94 (m, 1H), 1.90-1.83 (m, 1H), 1.78-1.71 (m, 1H), 1.11-0.82 (m, 6H).

MS (ESI): m/z 440.0 (M+1)⁺

### Example 172

### (3S,6S)-4-(3-(4-fluorophenyl)isoxazole-5-carbonyl)-3-isobutyl-6-propylpiperazin-2-one (189)

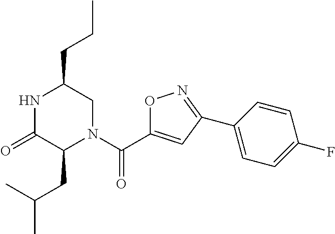

(3S,6S)-3-isobutyl-6-propylpiperazin-2-one 16 (50 mg, 0.25 mmol) and 3-(4-fluorophenyl)isoxazole-5-carboxylic acid (52 mg, 0.25 mmol) were coupled according to the procedure described for compound 71 to furnish 189 (77 mg, 79% yield) as white solid.

¹H NMR (400 MHz, CDCl₃): δ 7.81-7.77 (m, 2H), 7.21-7.16 (m, 2H), 6.86 (s, 1H), 6.04 (s, 1H), 5.29-5.26 (m, 1H), 4.89-4.78 (m, 1H), 3.70-3.63 (m, 1H), 3.17-2.83 (m, 1H), 1.91-1.73 (m, 3H), 1.54-1.36 (m, 4H), 1.08-0.77 (m, 9H).

MS (ESI): m/z 388.1 (M+1)⁺.

### Example 173

### (3S,6S)-4-[3-(4-Chloro-phenyl)-isoxazole-5-carbonyl]-3,6-diisobutyl-piperazin-2-one (190)

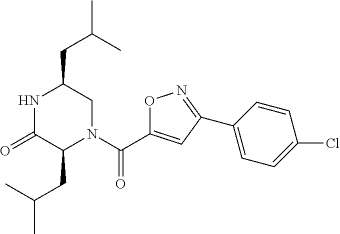

3,6-Di-isobutyl-piperazin-2-one substrate 7 (43 mg, 0.20 mmol) and 3-(4-chlorophenyl)-isoxazole-5-carboxylic acid (45 mg, 0.20 mmol) were coupled according to the procedure described for compound 71 to furnish 190 (60 mg, 70.9% yield) as a white solid.

¹H NMR (400 MHz, CDCl₃): δ 7.76 (dd, J=8 & 2.4 Hz, 1H), 7.76 (dd, J=8 & 3.2 Hz, 1H), 7.18 (s, 1H), 6.00 (s, 1H), 5.22 (dd, J=10.0 & 4.0 Hz, 1H), 4.92 (t, J=6.4 Hz, 1H), 4.72 (dd, J=12.8 & 4.0 Hz, 1H), 4.43 (dd, J=14.4 & 4.0 Hz, 1H), 3.77-3.68 (m, 1H), 3.17 (dd, J=14.4 & 11.2 Hz, 1H), 2.87 (dd, J=13.6 & 11.2 Hz, 1H), 1.91-1.66 (m, 4H), 1.42-1.35 (m, 2H) and 1.07-0.84 (m, 12H).

MS (ESI): m/z 418.1 [M+1]⁺

### Example 174

### (3S,6S)-6-(cyclopropylmethyl)-4-(3-(4-fluorophenyl)isoxazole-5-carbonyl)-3-isobutylpiperazin-2-one (191)

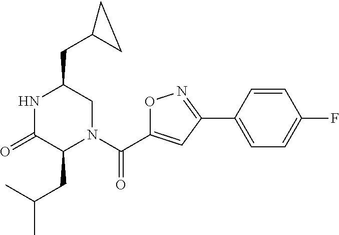

(3S,6S)-6-(cyclopropylmethyl)-3-isobutylpiperazin-2-one 13 (50 mg, 0.24 mmol) and 3-(4-fluorophenyl)isoxazole-5-carboxylic acid (49 mg, 0.24 mmol) were coupled according to the procedure described for compound 71 to furnish 191 (73 mg, 77% yield) as a white solid.

¹H NMR (400 MHz, CDCl₃): δ 7.81-7.77 (m, 2H), 7.21-7.16 (m, 2H), 6.86 (s, 1H), 6.13 (s, 1H), 5.31-5.28 (m, 1H), 4.94-4.82 (m, 1H), 3.82-3.74 (m, 1H), 3.22-2.88 (m, 1H), 1.89-1.55 (m, 4H), 1.29-1.19 (m, 1H), 1.08-0.77 (m, 6H), 0.73-0.52 (m, 3H), 0.21-0.09 (m, 2H).

MS (ESI): m/z 400.1 (M+1)⁺

### Example 175

### (3S,6S)-4-(3-(4-fluorophenyl)isoxazole-5-carbonyl)-3-isobutyl-6-propylpiperazin-2-one (192)

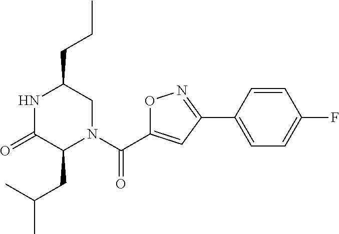

(3S,6S)-3-isobutyl-6-propylpiperazin-2-one 16 (50 mg, 0.25 mmol) and 3-(4-fluorophenyl)isoxazole-5-carboxylic acid (52 mg, 0.25 mmol) were coupled according to the procedure described for compound 71 to furnish 192 (77 mg, 79% yield) as a white solid.

¹H NMR (400 MHz, CDCl₃) δ 7.81-7.77 (m, 2H), 7.21-7.16 (m, 2H), 6.86 (s, 1H), 6.04 (s, 1H), 5.29-5.26 (m, 1H), 4.89-4.78 (m, 1H), 3.70-3.63 (m, 1H), 3.17-2.83 (m, 1H), 1.91-1.73 (m, 3H), 1.54-1.36 (m, 4H), 1.08-0.77 (m, 9H).

MS (ESI): m/z 388.1 (M+1)⁺.

### Example 176

### (3S,6S)-6-cyclohexyl-4-(3-(4-fluorophenyl)isoxazole-5-carbonyl)-3-isobutylpiperazin-2-one (193)

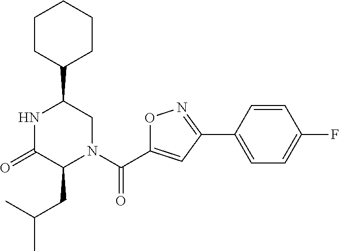

(3S,6S)-6-cyclohexyl-3-isobutylpiperazin-2-one 18 (50 mg, 0.21 mmol) and 3-(4-fluorophenyl)isoxazole-5-carboxylic acid (43 mg, 0.21 mmol) were coupled according to the procedure described for compound 71 to furnish 193 (61 mg, 68% yield) as a white solid.

¹H NMR (400 MHz, CDCl₃): δ 7.80-7.77 (m, 2H), 7.21-7.16 (m, 2H), 6.86 (s, 1H), 5.92 (s, 1H), 5.30-5.27 (m, 1H), 4.92-4.77 (m, 1H), 3.53-3.43 (m, 1H), 3.26-2.92 (m, 1H), 1.88-1.70 (m, 7H), 1.42-1.39 (m, 1H), 1.29-1.11 (m, 6H), 1.08-0.77 (m, 6H).

MS (ESI): m/z 428.1 (M+1)⁺

### Example 177

### (3S,6S)-6-cyclopentyl-4-(3-(4-fluorophenyl)isoxazole-5-carbonyl)-3-isobutylpiperazin-2-one (194)

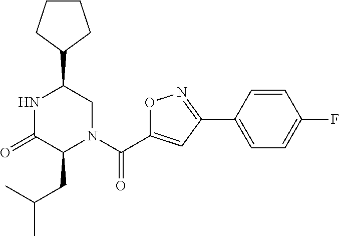

(3S,6S)-6-cyclopentyl-3-isobutylpiperazin-2-one 17 (50 mg, 0.22 mmol) and 3-(4-fluorophenyl)isoxazole-5-carboxylic acid (46 mg, 0.22 mmol) were coupled according to the procedure described for compound 71 to furnish 194 (71 mg, 77% yield) as a white solid.

¹H NMR (400 MHz, CDCl₃): δ 7.81-7.76 (m, 2H), 7.21-7.16 (m, 2H), 6.87 (s, 1H), 5.95 (s, 1H), 5.30-5.26 (m, 1H), 4.93-4.79 (m, 1H), 3.53-3.43 (m, 1H), 3.19-2.86 (m, 1H), 1.89-1.59 (m, 10H), 1.32-1.24 (m, 2H), 1.08-0.77 (m, 6H).

MS (ESI): m/z 414.2 (M+1)⁺

### Example 178

### (3S,6S)-3,6-Diisobutyl-4-((5-phenylisoxazol-3-yl)methyl)piperazin-2-one (195)

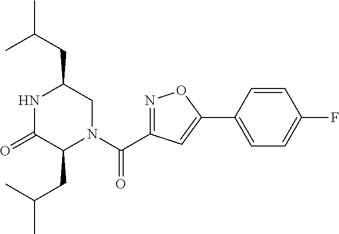

To a solution of 7 (100 mg, 0.47 mmol), 5-phenylisoxazole-3-carbaldehyde (81.4 mg, 0.47 mmol) in THF (5 mL) was added sodium triacetoxyborohydride (139.5 mg, 0.658 mmol). After 12 h stirring at RT, the mixture was quenched with NaOH (1N) and extracted with EtOAc (3×10 mL). The organic layers were combined, dried over anhydrous Na₂SO₄, filtered, and concentrated in vacuo. The residue was purified by flash silica gel chromatography to give the product 195 (104 mg, 60% yield) as a white solid.

¹H NMR (400 MHz, CDCl₃): δ 7.79-7.76 (2H, m), 7.79-7.43 (3H, m), 6.53 (1H, s), 5.82 (1H, s), 3.92 (1H, d, J=14 Hz), 3.85 (1H, d, J=14 Hz), 3.78-3.70 (1H, m), 3.23 (1H, dd, J=5, 8.2 Hz), 2.78-2.68 (2H, m), 1.96-1.86 (1H, m), 1.78-1.71 (1H, m), 1.66-1.56 (2H, m), 1.40-1.33 (1H, m), 1.29-1.22 (1H, m), 0.96-0.88 (12H, m).

MS (ESI): m/z 370.2 [M+1]⁺.

### Example 179

### (3S,6S)-4-((5-(4-Fluorophenyl)isoxazol-3-yl)methyl)-3,6-diisobutylpiperazin-2-one (196)

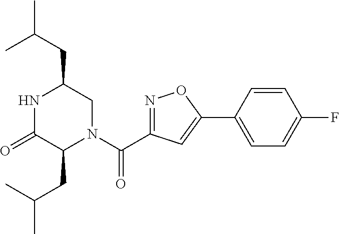

According to the method described for the preparation of compound 195, compound 7 (100 mg, 0.47 mmol) was coupled with 5-(4-fluorophenyl)isoxazole-3-carbaldehyde (90 mg, 0.47 mmol) to give the product 196 (73.5 mg, 40.3% yield) as a white solid.

¹H NMR (400 MHz, CDCl₃): δ 7.78-7.73 (2H, m), 7.17-7.12 (2H, m), 6.48 (1H, s), 6.04 (1H, s), 3.90 (1H, d, J=14 Hz), 3.83 (1H, d, J=14 Hz), 3.75-3.69 (1H, m), 3.21 (1H, dd, J=5, 8.2 Hz), 2.77-2.67 (2H, m), 1.95-1.84 (1H, m), 1.77-1.70 (1H, m), 1.66-1.55 (2H, m), 1.40-1.33 (1H, m), 1.29-1.22 (1H, m), 0.96-0.88 (12H, m).

MS (ESI): m/z 388.2 [M+1]⁺.

### Example 180

### (3S,6S)-4-((5-(4-Chlorophenyl)isoxazol-3-yl)methyl)-3,6-diisobutylpiperazin-2-one (197)

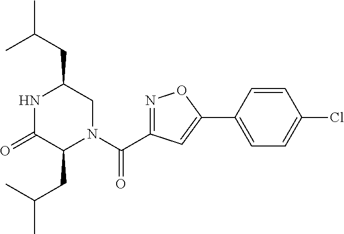

According to the method described for the preparation of compound 195, compound 7 (100 mg, 0.47 mmol) was coupled with 5-(4-chlorophenyl)isoxazole-3-carbaldehyde (97.6 mg, 0.47 mmol) to give the product 197 (19.4 mg, 10.2% yield) as a white solid.

¹H NMR (400 MHz, CDCl₃): δ 7.71 (2H, d, J=8.8 Hz), 7.45 (2H, d, J=8.8 Hz), 6.52 (1H, s), 5.75 (1H, s), 3.92 (1H, d, J=14 Hz), 3.84 (1H, d, J=14 Hz), 3.77-3.70 (1H, m), 3.22 (1H, dd, J=5.2, 8.4 Hz), 2.78-2.68 (2H, m), 1.95-1.84 (1H, m), 1.77-1.71 (1H, m), 1.66-1.55 (2H, m), 1.40-1.33 (1H, m), 1.29-1.22 (1H, m), 0.96-0.88 (12H, m).

MS (ESI): m/z 404.1 [M+1]⁺.

### Example 181

### (3S,6S)-4-((5-(4-Bromophenyl)isoxazol-3-yl)methyl)-3,6-diisobutylpiperazin-2-one (198)

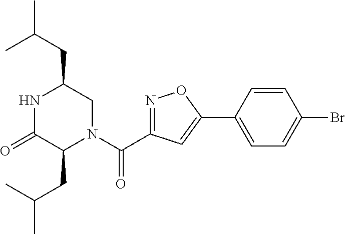

According to the method described for the preparation of compound 195, compound 7 (100 mg, 0.47 mmol) was coupled with 5-(4-bromophenyl)isoxazole-3-carbaldehyde (118.5 mg, 0.47 mmol) to give the product 198 (54.3 mg, 25.8% yield) as a white solid.

¹H NMR (400 MHz, CDCl₃): δ 7.65-7.58 (4H, m), 6.53 (1H, s), 6.05 (1H, s), 3.90 (1H, d, J=14 Hz), 3.83 (1H, d, J=14 Hz), 3.75-3.69 (1H, m), 3.20 (1H, dd, J=5, 8.2 Hz), 2.77-2.69 (2H, m), 1.92-1.84 (1H, m), 1.78-1.70 (1H, m), 1.65-1.55 (2H, m), 1.40-1.33 (1H, m), 1.29-1.22 (1H, m), 0.95-0.87 (12H, m).

MS (ESI): m/z 448.1 [M+1]⁺.

### Example 182

### (3S,6S)-3,6-Diisobutyl-4-((5-(p-tolyl)isoxazol-3-yl)methyl)piperazin-2-one (199)

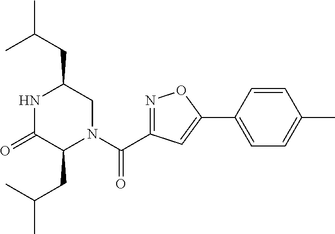

According to the method described for the preparation of compound 195, compound 7 (100 mg, 0.47 mmol) was coupled with 5-(p-tolyl)isoxazole-3-carbaldehyde (88 mg, 0.47 mmol) to give the product 199 (91.4 mg, 50.7% yield) as a white solid.

¹H NMR (400 MHz, CDCl₃): δ 7.66 (2H, d, J=8 Hz), 7.26 (2H, d, J=8 Hz), 6.47 (1H, s), 5.94 (1H, s), 3.90 (1H, d, J=14 Hz), 3.83 (1H, d, J=14 Hz), 3.76-3.69 (1H, m), 3.22 (1H, dd, J=5.2, 8.2 Hz), 2.77-2.69 (2H, m), 2.40 (3H, s), 1.94-1.84 (1H, m), 1.77-1.71 (1H, m), 1.66-1.56 (2H, m), 1.40-1.33 (1H, m), 1.29-1.22 (1H, m), 0.97-0.88 (12H, m).

MS (ESI): m/z 384.6 [M+1]⁺.

### Example 183

### (3S,6S)-3,6-Diisobutyl-4-((5-(4-methoxyphenyl)isoxazol-3-yl)methyl)piperazin-2-one (200)

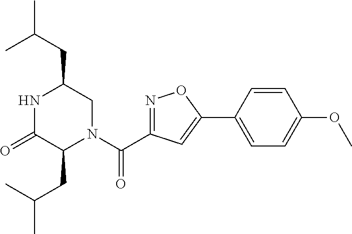

According to the method described for the preparation of compound 195, compound 7 (100 mg, 0.47 mmol) was coupled with 5-(4-methoxyphenyl)isoxazole-3-carbaldehyde (95.5 mg, 0.47 mmol) to give the product 200 (66.8 mg, 35.6% yield) as an yellow solid.

¹H NMR (400 MHz, CDCl₃): δ 7.71 (2H, d, J=8.8 Hz), 6.97 (2H, d, J=8.8 Hz), 6.40 (1H, s), 5.99 (1H, s), 3.91-3.81 (5H, m), 3.48 (1H, s), 3.22 (1H, dd, J=5.2, 8 Hz), 2.81-2.69 (2H, m), 1.94-1.84 (1H, m), 1.77-1.71 (1H, m), 1.66-1.56 (2H, m), 1.44-1.23 (2H, m), 0.96-0.88 (12H, m).

MS (ESI): m/z 400.2 [M+1]⁺.

### Example 184

### (3S,6S)-4-((5-(Furan-2-yl)isoxazol-3-yl)methyl)-3,6-diisobutylpiperazin-2-one (201)

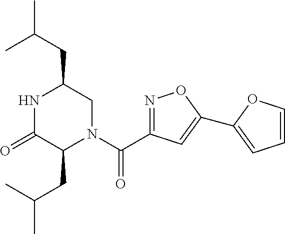

According to the method described for the preparation of compound 195, compound 7 (100 mg, 0.47 mmol) was coupled with 5-furan-2-yl-isoxazole-3-carbaldehyde (76.7 mg, 0.47 mmol) to give the product 201 (41 mg, 24.3% yield) as a pale yellow solid.

¹H NMR (400 MHz, CDCl₃): δ 7.54 (1H, s), 6.90 (1H, d, J=3.2 Hz), 6.54-6.53 (1H, m), 6.45 (1H, s), 5.92 (1H, s), 3.90 (1H, d, J=14 Hz), 3.82 (1H, d, J=14 Hz), 3.75-3.68 (1H, m), 3.20 (1H, dd, J=5, 8.2 Hz), 2.76-2.65 (2H, m), 1.92-1.54 (4H, m), 1.40-1.22 (2H, m), 0.95-0.87 (12H, m).

MS (ESI): m/z 360.3 [M+1]⁺.

### Example 185

### (3S,6S)-3,6-Diisobutyl-4-((5-(thiophen-2-yl)isoxazol-3-yl)methyl)piperazin-2-one (202)

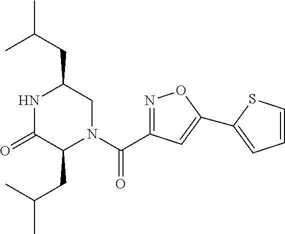

According to the method described for the preparation of compound 195, compound 7 (100 mg, 0.47 mmol) was coupled with 5-thiophen-2-yl-isoxazole-3-carbaldehyde (84.2 mg, 0.47 mmol) to give the product 202 (38 mg, 21.5% yield) as a white solid.

¹H NMR (400 MHz, CDCl₃): δ 7.51 (1H, d, J=4 Hz), 7.45 (1H, d, J=4.8 Hz), 7.14-7.12 (1H, m), 6.39 (1H, s), 5.79 (1H, s), 3.89 (1H, d, J=14 Hz), 3.82 (1H, d, J=14 Hz), 3.76-3.69 (1H, m), 3.21 (1H, dd, J=5.6, 8.2 Hz), 2.77-2.67 (2H, m), 1.92-1.54 (4H, m), 1.40-1.22 (2H, m), 0.96-0.88 (12H, m).

MS (ESI): m/z 376.1 [M+1]⁺.

### Example 186

### (3S,6S)-6-Cyclopropylmethyl-4-[5-(4-fluoro-phenyl)-isoxazol-3-ylmethyl]-3-isobutyl-piperazin-2-one (203)

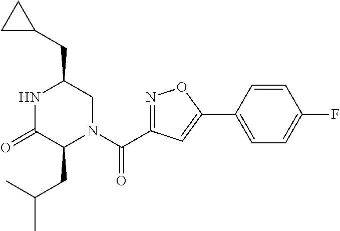

According to the method described for the preparation of compound 195, compound 13 (100 mg, 0.47 mmol) was coupled with 5-(4-fluorophenyl)isoxazole-3-carbaldehyde (90 mg, 0.47 mmol) to give the product 203 (73.5 mg, 40.3% yield) as a white solid.

¹H NMR (400 MHz, CDCl₃): δ 7.77-7.74 (m, 2H), 7.17-7.13 (m, 2H), 6.48 (s, 1H), 6.23 (s, 1H), 3.93-3.65 (m, 3H), 3.21 (dd, J=8.4 & 5.6 Hz, 1H), 2.82-2.65 (m, 2H), 1.86-1.68 (m, 3H), 1.46-140 (m, 1H), 1.27-1.20 (m, 1H), 0.95-0.87 (m, 6H), 0.65-0.41 (m, 3H) and 0.14-0.01 (m, 2H).

MS (ESI): m/z 386.1 [M+1]⁺.

### Example 187

### (3S,6S)-6-(2,2-Dimethyl-propyl)-4-[5-(4-fluoro-phenyl)-isoxazol-3-ylmethyl]-3-isobutyl-piperazin-2-one (204)

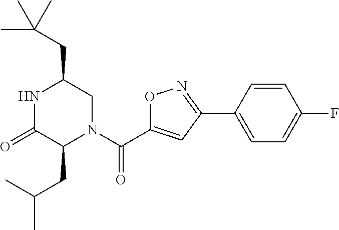

According to the method described for the preparation of compound 195, compound 15 (100 mg, 0.47 mmol) was coupled with 5-(4-fluorophenyl)isoxazole-3-carbaldehyde (90 mg, 0.47 mmol) to give the product 204 (73.5 mg, 40.3% yield) as a white solid.

¹H NMR (400 MHz, CDCl₃): δ 7.78-7.75 (m, 2H), 7.18-7.14 (m, 2H), 6.48 (s, 1H), 5.73 (s, 1H), 3.96-3.75 (m, 3H), 3.21 (dd, J=8.4 & 4.8 Hz, 1H), 2.79-2.65 (m, 2H), 1.92-1.85 (m, 1H), 1.76-1.57 (m, 2H), 1.35-1.32 (m, 2H), 0.96-0.87 (m, 15H).

MS (ESI): m/z 402.2 [M+1]⁺.

### Example 188

### (3S,6S)-6-(tert-butyl)-4-((5-(4-fluorophenyl)isoxazol-3-yl)methyl)-3-isobutylpiperazin-2-one (205)

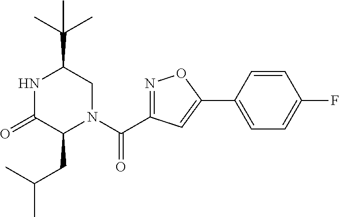

According to the method described for the preparation of compound 195, compound 14 (50 mg, 0.24 mmol) was coupled with 5-(4-fluorophenyl)isoxazole-3-carbaldehyde (45 mg, 0.24 mmol) to give the product 205 (73.5 mg, 40.3% yield) as a white solid.

¹HNMR (400 MHz, CDCl₃): δ 7.78-7.75 (m, 2H), 7.18-7.14 (m, 2H), 6.48 (s, 1H), 5.78 (s, 1H), 3.99-3.77 (m, 2H), 3.48-3.44 (m, 1H), 3.25-3.21 (m, 1H), 2.91-2.69 (m, 2H), 1.92-1.85 (m, 1H), 1.67-1.51 (m, 2H), 0.96-0.85 (m, 15H).

MS (ESI): m/z 388.4 (M+1)⁺

### Example 189

### (3S,6S)-4-((5-(4-fluorophenyl)isoxazol-3-yl)methyl)-3-isobutyl-6-propylpiperazin-2-one (206)

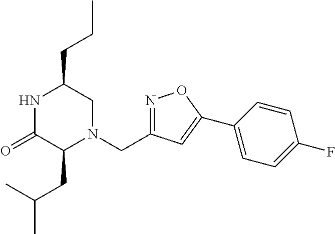

(3S,6S)-3-isobutyl-6-propylpiperazin-2-one 16 (50 mg, 0.25 mmol) and 5-(4-fluorophenyl)isoxazole-3-carbaldehyde (48 mg, 0.25 mmol) were coupled according to the procedure described for the preparation of compound 195 to furnish 206 (68 mg, 72% yield) as a white solid.

¹H NMR (400 MHz, CDCl₃): δ 7.78-7.75 (m, 2H), 7.19-7.09 (m, 2H), 6.48 (s, 1H), 6.30 (s, 1H), 3.92-3.81 (m, 2H), 3.67-3.64 (m, 1H), 3.23-3.20 (m, 1H), 2.80-2.68 (m, 2H), 1.93-1.23 (m, 7H), 1.04-0.85 (m, 9H).

MS (ESI): m/z 374.2 (M+1)⁺

### Example 190

### (3S,6S)-6-cyclohexyl-4-((5-(4-fluorophenyl)isoxazol-3-yl)methyl)-3-isobutylpiperazin-2-one (207)

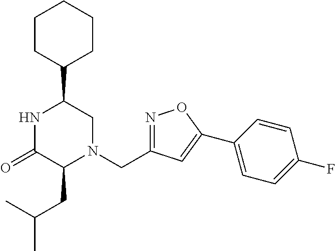

(3S,6S)-6-cyclohexyl-3-isobutylpiperazin-2-one 18 (50 mg, 0.21 mmol) and 5-(4-fluorophenyl)isoxazole-3-carbaldehyde (40 mg, 0.21 mmol) were coupled according to the procedure described for the preparation of compound 195 to furnish 207 (61 mg, 70% yield) as a white solid.

¹H NMR (400 MHz, CDCl₃): δ 7.78-7.75 (m, 2H), 7.18-7.14 (m, 2H), 6.47 (s, 1H), 5.88 (s, 1H), 3.90-3.79 (m, 2H), 3.42-3.38 (m, 1H), 3.23-3.19 (m, 1H), 2.91-2.86 (m, 1H), 2.69-2.65 (m, 1H), 1.91-1.86 (m, 1H), 1.78-1.56 (m, 9H), 1.42-1.34 (m, 1H), 1.25-1.05 (m, 3H), 0.96 (d, 3H, J=6.8 Hz), 0.90 (d, 3H, J=6.8 Hz).

MS (ESI): m/z 414.2 (M+1)⁺

### Example 191

### (3S,6S)-6-cyclopentyl-4-((5-(4-fluorophenyl)isoxazol-3-yl)methyl)-3-isobutylpiperazin-2-one (208)

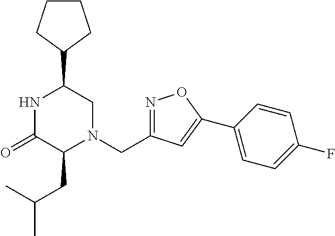

(3S,6S)-6-cyclopentyl-3-isobutylpiperazin-2-one 17 (50 mg, 0.22 mmol) and 5-(4-fluorophenyl)isoxazole-3-carbaldehyde (43 mg, 0.22 mmol) were coupled according to the procedure described for the preparation of compound 195 to furnish 208 (55 mg, 61% yield) as white solid.

¹H NMR (400 MHz, CDCl₃): δ 7.78-7.75 (m, 2H), 7.18-7.14 (m, 2H), 6.48 (s, 1H), 5.95 (s, 1H), 3.93-3.80 (m, 2H), 3.45-3.41 (m, 1H), 3.23-3.20 (m, 1H), 2.83-2.69 (m, 2H), 1.91-1.81 (m, 3H), 1.76-1.69 (m, 1H), 1.65-1.49 (m, 6H), 1.24-1.18 (m, 1H), 1.07-1.02 (m, 1H), 0.96 (d, 3H, J=7.2 Hz), 0.89 (d, 3H, J=7.2 Hz);

MS (ESI): m/z 400.1 (M+1)⁺

### Example 192

### (3S,6S)-4-((5-(4-chlorophenyl)isoxazol-3-yl)methyl)-6-(cyclopropylmethyl)-3-isobutylpiperazin-2-one (209)

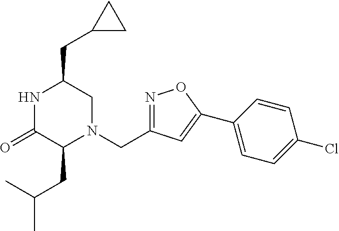

(3S,6S)-6-(cyclopropylmethyl)-3-isobutylpiperazin-2-one 13 (50 mg, 0.24 mmol) and 5-(4-chlorophenyl)isoxazole-3-carbaldehyde (50 mg, 0.24 mmol) were coupled according to the procedure described for the preparation of compound 195 to furnish 209 (47 mg, 49% yield) as a white solid.

¹H NMR (400 MHz, CDCl₃): δ 7.72-7.69 (m, 2H), 7.46-7.42 (m, 2H), 6.52 (s, 1H), 5.98 (s, 1H), 3.94-3.82 (m, 2H), 3.78-3.76 (m, 1H), 3.24-3.21 (m, 1H), 2.84-2.72 (m, 2H), 1.92-1.85 (m, 1H), 1.77-1.71 (m, 1H), 1.65-1.58 (m, 1H), 1.49-1.43 (m, 1H), 1.25-1.18 (m, 1H), 0.95-0.87 (m, 6H), 0.63-0.44 (m, 3H), 0.16-0.01 (m, 2H).

MS (ESI): m/z 402.2 (M+1)⁺

### Example 193

### (3S,6S)-4-((5-(4-chlorophenyl)isoxazol-3-yl)methyl)-3-isobutyl-6-propylpiperazin-2-one (210)

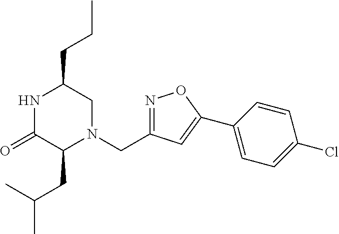

(3S,6S)-3-isobutyl-6-propylpiperazin-2-one 16 (50 mg, 0.25 mmol) and 5-(4-chlorophenyl)isoxazole-3-carbaldehyde (52 mg, 0.25 mmol) were coupled according to the procedure described for the preparation of compound 195 to furnish 210 (44 mg, 45% yield) as a white solid.

¹H NMR (400 MHz, CDCl₃): δ 7.72-7.70 (m, 2H), 7.45-7.43 (m, 2H), 6.52 (s, 1H), 5.79 (s, 1H), 3.93-3.81 (m, 2H), 3.68-3.64 (m, 1H), 3.23-3.20 (m, 1H), 2.79-2.68 (m, 2H), 1.91-1.86 (m, 1H), 1.76-1.62 (m, 2H), 1.44-1.25 (m, 4H), 0.96-0.87 (m, 9H).

MS (ESI): m/z 390.1 (M+1)⁺

### Example 194

### (3S,6S)-4-((5-(4-chlorophenyl)isoxazol-3-yl)methyl)-6-cyclohexyl-3-isobutylpiperazin-2-one (211)

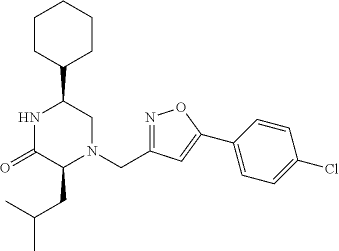

(3S,6S)-6-cyclohexyl-3-isobutylpiperazin-2-one 18 (50 mg, 0.21 mmol) and 5-(4-chlorophenyl)isoxazole-3-carbaldehyde (43 mg, 0.21 mmol) were coupled according to the procedure described for the preparation of compound 195 to furnish 211 (53 mg, 59% yield) as a white solid.

¹H NMR (400 MHz, CDCl₃): δ 7.72-7.69 (m, 2H), 7.46-7.42 (m, 2H), 6.52 (s, 1H), 5.86 (s, 1H), 3.91-3.79 (m, 2H), 3.42-3.38 (m, 1H), 3.22-3.19 (m, 1H), 2.91-2.85 (m, 1H), 2.69-2.64 (m, 1H), 1.91-1.86 (m, 1H), 1.78-1.55 (m, 8H), 1.39-1.37 (m, 1H), 1.22-0.99 (m, 4H), 0.96-0.87 (m, 6H).

MS (ESI): m/z 430.1 (M+1)⁺

### Example 195

### (3S,6S)-4-((5-(4-chlorophenyl)isoxazol-3-yl)methyl)-6-cyclopentyl-3-isobutylpiperazin-2-one (212)

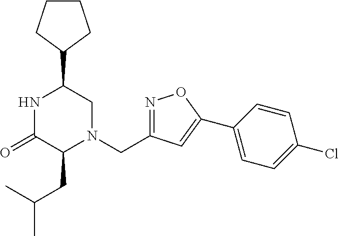

(3S,6S)-6-cyclopentyl-3-isobutylpiperazin-2-one 17 (50 mg, 0.22 mmol) and 5-(4-chlorophenyl)isoxazole-3-carbaldehyde (46 mg, 0.22 mmol) were coupled according to the procedure described for the preparation of compound 195 to furnish 212 (57 mg, 61% yield) as a white solid.

¹H NMR (400 MHz, CDCl₃): δ 7.72-7.70 (m, 2H), 7.46-7.42, 2H), 6.52 (s, 1H), 5.85 (s, 1H), 3.92-3.80 (m, 2H), 3.46-3.41 (m, 1H), 3.23-3.20 (m, 1H), 2.83-2.69 (m, 2H), 1.91-1.50 (m, 10H), 1.24-1.01 (m, 2H), 0.96-0.87 (m, 6H).

MS (ESI): m/z 416.2 (M+1)⁺

### Example 196

### (3S,6S)-4-((5-(4-fluorophenyl)isoxazol-3-yl)methyl)-3-isobutyl-6-isopropylpiperazin-2-one (213)

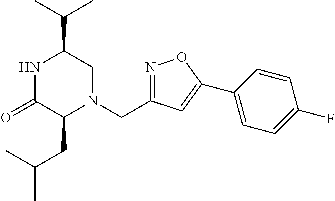

(3S,6S)-3-isobutyl-6-isopropylpiperazin-2-one 8 (50 mg, 0.25 mmol) and 5-(4-fluorophenyl)isoxazole-3-carbaldehyde (48 mg, 0.25 mmol) were coupled according to the procedure described for the preparation of compound 195 to furnish 213 (53 mg, 56% yield) as a white solid.

¹H NMR (400 MHz, CDCl₃): δ 7.78-7.75 (m, 2H), 7.25-7.14 (m, 2H), 6.48 (s, 1H), 5.82 (s, 1H), 3.92-3.80 (m, 2H), 3.42-3.38 (m, 1H), 3.23-3.20 (m, 1H), 2.89-2.83 (m, 1H), 2.71-2.66 (m, 1H), 1.93-1.84 (m, 1H), 1.74-1.57 (m, 3H), 0.96-0.86 (m, 12H).

MS (ESI): m/z 374.2 (M+1)⁺

### Example 197

### (3S,6S)-6-(((R)-sec-butyl)-4-((5-(4-fluorophenyl)isoxazol-3-yl)methyl)-3-isobutylpiperazin-2-one (214)

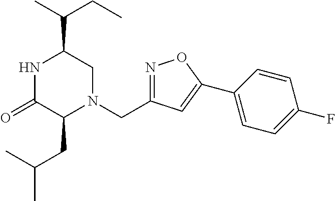

(3S,6S)-6-((R)-sec-butyl)-3-isobutylpiperazin-2-one 9 (50 mg, 0.24 mmol) and 5-(4-fluorophenyl)isoxazole-3-carbaldehyde (45 mg, 0.24 mmol) were coupled according to the procedure described for the preparation of compound 195 to furnish 214 (13 mg, 14% yield) as a white solid.

¹H NMR (400 MHz, CDCl₃): δ 7.80-7.76 (m, 2H), 7.18-7.14 (m, 2H), 6.44 (s, 1H), 5.69 (s, 1H), 3.94 (d, 1H, J=14.4 Hz), 3.58 (d, 1H, J=14.4 Hz), 3.47-3.45 (m, 1H), 3.15-3.13 (m, 1H), 2.90-2.86 (m, 1H), 2.34-2.29 (m, 1H), 1.96-1.88 (m, 3H), 1.39-1.36 (m, 2H), 1.15-1.08 (m, 1H), 1.01-0.84 (m, 12H).

MS (ESI): m/z 388.2 (M+1)⁺

### Example 198

### (3S,6S)-4-((5-(4-fluorophenyl)isoxazol-3-yl)methyl)-3-isobutyl-6-phenylpiperazin-2-one (215)

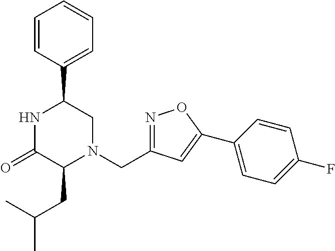

(3S,6S)-3-isobutyl-6-phenylpiperazin-2-one 11 (50 mg, 0.22 mmol) and 5-(4-fluorophenyl)isoxazole-3-carbaldehyde (41 mg, 0.22 mmol) were coupled according to the procedure described for the preparation of compound 195 to furnish 215 (47 mg, 54% yield) as a white solid.

¹H NMR (400 MHz, CDCl₃): δ 7.74-7.71 (m, 2H), 7.36-7.25 (m, 6H), 7.17-7.13 (m, 2H), 6.35 (s, 1H), 5.90 (s, 1H), 4.90-4.86 (m, 1H), 4.08-3.90 (m, 2H), 3.35-3.32 (m, 1H), 2.99-2.85 (m, 2H), 1.95-1.78 (m, 2H), 1.74-1.67 (m, 1H), 0.98 (d, 3H, J=6.8 Hz) 0.89 (d, 3H, J=6.8 Hz).

MS (ESI): m/z 408.1 (M+1)⁺

### Example 199

### (3S,6S)-6-cyclopropyl-4-((5-(4-fluorophenyl)isoxazol-3-yl)methyl)-3-isobutylpiperazin-2-one (216)

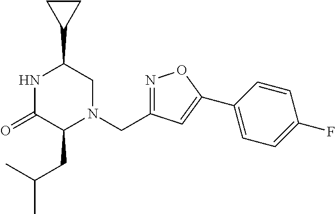

(3S,6S)-6-cyclopropyl-3-isobutylpiperazin-2-one 13 (50 mg, 0.25 mmol) and 5-(4-fluorophenyl)isoxazole-3-carbaldehyde (49 mg, 0.25 mmol) were coupled according to the procedure described for the preparation of compound 195 to furnish 216 (49 mg, 52% yield) as a white solid.

¹H NMR (400 MHz, CDCl₃): δ 7.78-7.75 (m, 2H), 7.18-7.14 (m, 2H), 6.48 (s, 1H), 5.87 (s, 1H), 3.91-3.77 (m, 2H), 3.24-3.21 (s, 1H), 2.98-2.92 (s, 1H), 5.29-5.26 (m, 1H), 4.95-4.86 (m, 1H), 3.98-3.90 (m, 1H), 3.25-3.00 (m, 1H), 2.98-2.92 (m, 1H), 2.84-2.78 (m, 1H), 1.94-1.87 (m, 1H), 1.79-1.72 (m, 1H), 1.66-1.59 (m, 1H), 0.97 (d, 3H, J=6.4 Hz), 0.89 (d, 3H, J=6.4 Hz), 0.84-0.78 (m, 1H), 0.57-0.47 (m, 2H), 0.24-0.15 (m, 2H).

MS (ESI): m/z 372.4 (M+1)⁺

### Example 200

### (3S,6R)-4-[5-(4-Fluoro-phenyl)-isoxazol-3-ylmethyl]-3-isobutyl-6-methylsulfanylmethyl-piperazin-2-one (217)

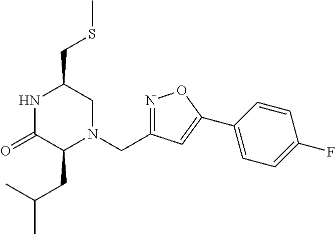

(3S,6R)-3-isobutyl-6-((methylthio)methyl)piperazin-2-one 10 (50 mg, 0.23 mmol) and 5-(4-fluorophenyl)isoxazole-3-carbaldehyde (48 mg, 0.23 mmol) were coupled according to the procedure described for the preparation of compound 195 to furnish 217 (47 mg, 50% yield) as a white solid.

¹H NMR (400 MHz, CDCl₃): δ 7.84-7.78 (m, 2H), 7.46 (bs, 1H), 7.20-7.15 (m, 2H), 4.48-4.22 (m, 2H), 3.81-3.68 (m, 1H), 3.43-3.10 (m, 2H), 2.97-2.78 (m, 1H), 2.75 (s, 1H), 2.73 (s, 1H), 2.53 (dd, J=8.0, 14.0 Hz, 1H), 2.17 (s, 3H), 2.05-1.96 (m, 2H), 1.85-1.78 (m, 1H), 0.99-0.91 (m, 6H).

MS (ESI): m/z 392.4 (M+1)⁺

### Example 201

### (3S,6R)-4-((5-(4-chlorophenyl)isoxazol-3-yl)methyl)-3-isobutyl-6-((methylthio)methyl)piperazin-2-one (218)

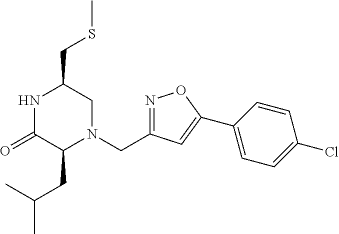

(3S,6R)-3-isobutyl-6-((methylthio)methyl)piperazin-2-one 10 (50 mg, 0.23 mmol) and 5-(4-chlorophenyl)isoxazole-3-carbaldehyde (48 mg, 0.23 mmol) were coupled according to the procedure described for the preparation of compound 195 to furnish 218 (47 mg, 50% yield) as a white solid.

¹H NMR (400 MHz, CDCl₃): δ 7.71, (m, 2H), 7.45 (m, 2H), 6.51 (s, 1H), 6.24 (s, 1H) 3.92-3.83 (m, 2H), 3.73 (m, 1H), 3.24 (m, 1H) 2.88-2.73 (m, 2H), 2.64 (dd, 1H, J=16.0, 4.0 Hz), 2.41 (dd, 1H, J=13.6, 9.2 Hz), 2.09 (s, 3H) 1.91-1.86 (m, 1H), 1.79-1.69 (m, 1H), 1.67-1.62 (m, 1H) 0.95 (d, 3H, J=6.8 Hz), 0.91 (d, 3H, J=6.8 Hz).

MS (ESI): m/z 408.1 (M+1).

### Example 202

### (3S,6S)-4-((5-(4-chlorophenyl)isoxazol-3-yl)methyl)-3-isobutyl-6-phenylpiperazin-2-one (219)

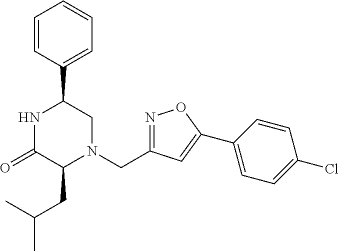

(3S,6S)-3-isobutyl-6-phenylpiperazin-2-one 11 (50 mg, 0.215 mmol) and 5-(4-chlorophenyl)isoxazole-3-carbaldehyde (45 mg, 0.22 mmol) were coupled according to the procedure described for the preparation of compound 195 to furnish 219 (42 mg, 45% yield) as a white solid.

¹H NMR (400 MHz, CDCl₃): δ 7.66 (d, 2H, J=2.4 Hz), 7.43 (d, 2H, J=2.4 Hz), 7.4-7.25 (m, 5H), 6.38 (s, 1H), 6.05 (s, 1H), 4.87 (dd, 1H, J=9.6, 5.2 Hz), 3.98 (dd, 2H, J=56.8, 14.0 Hz), 3.33 (dd, 1H, J=8.8, 4.8 Hz), 2.99-2.84 (m, 2H), 1.94-1.88 (m, 1H), 1.84-1.78 (m, 1H), 1.74-1.67 (m, 1H), 0.97 (d, 3H) 0.89 (d, 3H).

MS (ESI): m/z 424.1 (M+1)⁺

### Example 203

### (3S,6S)-4-(4-fluoro-2-(trifluoromethyl)benzyl)-3,6-diisobutylpiperazin-2-one (220)

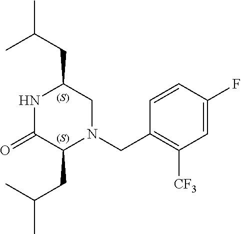

A mixture of 3,6-Diisobutyl-piperazin-2-one 7 (200 mg, 0.94 mmol) in DMF (10 mL), was added 1-Bromomethyl-4-fluoro-2-trifluoromethyl-benzene (240 mg, 0.94 mmol), K₂CO₃ (130 mg, 0.94 mmol) and NaI (141 mg, 0.94 mmol). The mixture was stirred for 8 hours at RT and then the mixture was washed with water, dried over Na₂SO₄, filtrated and concentrated, the residue was separated by prep HPLC under acid condition to give desired product 220 (24 mg, 6.58% yield) as an oil.

¹H NMR (400 MHz, DMSO-d₆): δ 7.80 (dd, J=14 Hz, 1H), 7.69 (s, 1H), 7.56-7.61 (m, 2H), 3.83 (dd, J=35.2 Hz, 2H), 3.51 (t, J=6 Hz, 1H), 2.84-2.87 (m, 1H), 2.61-2.62 (m, 2H), 1.78 (m, 1H), 1.64-1.66 (m, 1H), 1.51-1.52 (m, 1H), 1.33-1.43 (m, 2H), 1.22-1.24 (m, 1H), 0.63-0.85 (m, 12H).

MS (ESI): m/z 389.2 [M+1]⁺.

### Example 204

### (3S,6S)-4-(4-fluoro-3-(trifluoromethyl)benzyl)-3,6-diisobutylpiperazin-2-one (221)

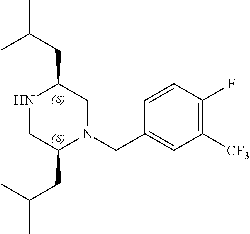

3,6-Diisobutyl-piperazin-2-one 7 (200 mg, 0.94 mmol) was on N-alkylation with 4-Bromomethyl-1-fluoro-2-trifluoromethyl-benzene (240 mg, 0.94 mmol) by the method described for the preparation of compound 220 furnished the desired product 221 (85 mg, 23.3% yield) as an oil.

¹H NMR (400 MHz, DMSO-d₆): δ 7.67-7.74 (m, 3H), 7.48 (dd, J=6 Hz, 5.4 Hz, 1H), 3.83 (d, J=13.6 Hz, 1H), 3.68 (d, J=13.6 Hz, 1H), 3.52 (t, J=6 Hz, 1H), 2.78-2.81 (m, 1H), 2.60-2.61 (m, 2H), 1.62-1.83 (m, 2H), 1.30-1.59 (m, 3H), 1.15-1.30 (m, 1H), 0.60-0.90 (m, 12H).

MS (ESI): m/z 389.2 [M+1]⁺.

### Example 205

### (3S,6S)-4-(2,5-difluorobenzyl)-3,6-diisobutylpiperazin-2-one (222)

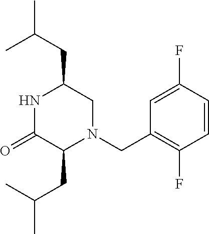

3,6-Diisobutyl-piperazin-2-one 7 (200 mg, 0.94 mmol) was on N-alkylation with 2-Bromomethyl-1,4-difluoro-benzene (194 mg, 0.94 mmol) by the method described for the preparation of compound 220 furnished the desired product 222 (90 mg, 28.3% yield) as an oil.

¹H NMR (400 MHz, DMSO-d₆): δ 7.68 (s, 1H), 7.12-7.30 (m, 3H), 3.78 (d, J=14.0 Hz, 1H), 3.66 (d, J=14.4 Hz, 1H), 3.53 (t, J=5.6 Hz 1H), 2.82-2.85 (m, 1H), 2.61-2.63 (m, 2H), 1.73-1.74 (m, 1H), 1.66-1.68 (m, 1H), 1.63-1.64 (m, 1H), 1.42-1.63 (m, 2H), 1.22-1.34 (m, 1H), 0.66-0.83 (m, 12H)

### Example 206

### (3S,6S)-4-(3,5-Bis-trifluoromethyl-benzyl)-3,6-diisobutyl-piperazin-2-one (223)

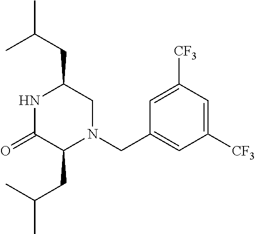

(3S,6S)-3,6-Diisobutyl-piperazin-2-one 7 (200 mg, 0.94 mmol) was on N-alkylation with 1-Bromomethyl-3,5-bis-trifluoromethyl-benzene (286 mg, 0.94 mmol) by the method described for the preparation of compound 220 furnished the desired product 223 (60 mg, 14.6% yield) as an oil.

¹H NMR (400 MHz, DMSO-d₆): δ 8.02 (s, 1H), 7.99 (s, 1H), 7.71 (s, 1H), 3.96 (d, J=14.4 Hz, 1H), 3.82 (d, J=14.0 Hz, 1H), 3.50-3.53 (m, 1H), 2.79 (dd, J₁=5.2 Hz, J_{z}=7.6 Hz, 1H), 2.62-2.64 (m, 2H), 1.63-1.72 (m, 2H), 1.50-1.52 (m, 1H), 1.36-1.43 (m, 2H), 1.19-1.24 (m, 1H), 0.79-1.24 (m, 12H).

MS (ESI): m/z 439.2 [M+1]⁺

### Example 207

### (3S,6S)-4-(2,6-Difluoro-benzyl)-3,6-diisobutyl-piperazin-2-one (224)

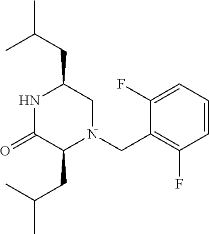

(3S,6S)-3,6-Diisobutyl-piperazin-2-one 7 (150 mg, 0.706 mmol) was on N-alkylation with 2-Bromomethyl-1,3-difluoro-benzene (146 mg, 0.706 mmol) by the method described for the preparation of compound 220 furnished the desired product 224 (32 mg, 37.58% yield) as an oil.

¹H NMR (400 MHz, DMSO-d₆): δ 7.64 (s, 1H), 7.37-7.44 (m, 1H), 7.07-7.13 (m, 2H), 3.81 (d, J=17.6 Hz, 1H), 3.72 (d, J=12.8 Hz, 1H), 3.55-3.65 (m, 1H), 2.83 (dd, J₁=4.8 Hz, J₂=9.2 Hz, 1H), 2.60-2.70 (m, 2H), 1.50-1.70 (m, 3H), 1.23-1.36 (m, 2H), 1.11-1.19 (m, 1H), 0.78-0.83 (m, 9H), 0.56-0.58 (m, 3H).

MS (ESI): m/z 339.4 [M+1]⁺

### Example 208

### (3S,6S)-4-(4-Ethyl-benzyl)-3,6-diisobutyl-piperazin-2-one (225)

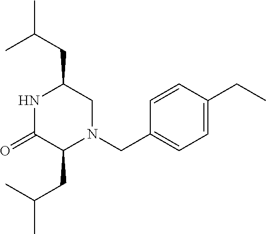

(3S,6S)-3,6-Diisobutyl-piperazine-2-one 7 (150 mg, 0.706 mmol) was on N-alkylation with 1-Chloromethyl-4-ethyl-benzene (109.2 mg, 0.706 mmol) by the method described for the preparation of compound 220 furnished the desired product 225 (57.21 mg, 24.73% yield) as a white solid.

¹H NMR (400 MHz, DMSO-d₆): δ 7.60 (s, 1H), 7.17 (d, J=8.8 Hz, 1H), 7.12 (d, J=8.0 Hz, 1H), 3.69-3.72 (m, 1H), 3.46-3.49 (m, 2H), 2.79-2.81 (m, 1H), 2.50-2.53 (m, 4H), 1.61-1.79 (m, 2H), 1.28-1.48 (m, 3H), 1.15-1.19 (m, 1H), 1.11 (t, J=7.2 Hz, 3H), 0.64-0.80 (m, 12H).

MS (ESI): m/z 331.4 [M+1]⁺

### Example 209

### (3S,6S)-4-(3,4-Dichloro-benzyl)-3,6-diisobutyl-piperazin-2-one (226)

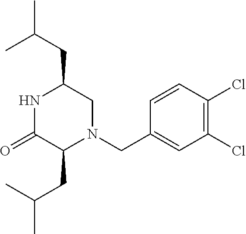

(3S,6S)-3,6-Diisobutyl-piperazine-2-one 7 (207 mg, 1.059 mmol) was on N-alkylation with 1,2-Dichloro-4-chloromethyl-benzene (150 mg, 0.706 mmol) by the method described for the preparation of compound 220 furnished the desired product 226 (151 mg, 57.6% yield) as a white solid.

¹H NMR (400 MHz, DMSO-d₆): δ 7.68 (s, 1H), 7.59 (dd, J₁=8.0 Hz, J₂=2.0 Hz, 1H) 7.57 (s, 1H), 7.31 (dd, J₁=8.0 Hz, J₂=2.0 Hz, 1H), 3.76 (d, J=13.6 Hz, 1H), 3.60 (d, J=13.6 Hz, 1H), 3.49-3.52 (m, 1H), 2.80 (dd, J=5.2 Hz, 7.6 Hz, 1H), 2.54-2.59 (m, 2H), 1.74-1.78 (m, 1H), 1.63-1.69 (m, 1H), 1.37-1.50 (m, 2H), 1.30-1.35 (m, 1H), 1.19-1.25 (m, 1H), 0.76-0.82 (m, 9H), 0.67-0.69 (m, 3H).

MS (ESI): m/z 371.0 [M+1]⁺

### Example 210

### (3S,6S)-4-(3-Chloro-4-fluoro-benzyl)-3,6-diisobutyl-piperazin-2-one (227)

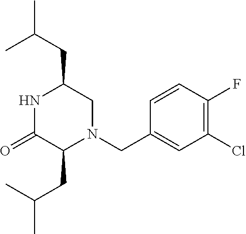

(3S,6S)-3,6-Diisobutyl-piperazine-2-one 7 (150 mg, 0.706 mmol) was on N-alkylation with 4-Bromomethyl-2-chloro-1-fluoro-benzene (236 mg, 1.059 mmol) by the method described for the preparation of compound 220 furnished the desired product 227 (39 mg, 15.6% yiled) as a white solid.

¹H NMR (400 MHz, DMSO-d₆): δ 7.65 (s, 1H), 7.51 (dd, J₁=7.2 Hz, J₂=1.6 Hz, 1H), 7.30-7.39 (m, 2H), 3.75 (d, J=13.6 Hz, 1H), 3.58 (d, J=13.2 Hz, 1H), 3.46-3.49 (m, 1H), 2.80 (dd, J=5.2 Hz, 7.2 Hz, 1H), 2.55 (m, 2H), 1.74-1.79 (m, 1H), 1.62-1.69 (m, 1H), 1.39-1.50 (m, 2H), 1.29-1.33 (m, 1H), 1.20-1.24 (m, 1H), 0.75-0.83 (m, 9H), 0.66-0.69 (m, 3H).

MS (ESI): m/z 355.3 [M+1]⁺

### Example 211

### (3S,6S)-3,6-Diisobutyl-4-(4-trifluoromethoxy-benzyl)-piperazin-2-one (228)

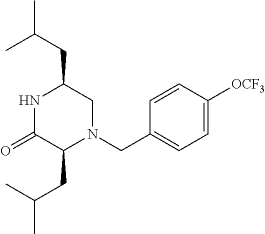

(3S,6S)-3,6-Diisobutyl-piperazine-2-one 7 (150 mg, 0.706 mmol) was on N-alkylation with 1-Bromomethyl-4-trifluoromethoxy-benzene (180 mg, 0.706 mmol) by the method described for the preparation of compound 220 furnished the desired product 228 (77 mg, 28.2% yiled) as a white solid.

¹H NMR (400 MHz, DMSO-d₆): δ 7.67 (s, 1H), 7.43 (d, J=8.8 Hz, 2H), 7.32 (d, J=8.0 Hz, 2H), 3.78 (d, J=13.2 Hz, 1H), 3.60 (d, J=13.2 Hz, 1H), 3.44-3.49 (m, 1H), 2.79-2.82 (dd, J=5.2 Hz, J₂=7.6 Hz, 1H), 2.54-2.58 (m, 2H), 1.74-1.80 (m, 1H), 1.63-1.69 (m, 1H), 1.30-1.50 (m, 3H), 1.20-1.25 (m, 1H), 0.76-0.84 (m, 9H), 0.63-0.67 (m, 3H).

MS (ESI): m/z 387.3 [M+1]⁺

### Example 212

### (3S,6S)-4-(2-Chloro-5-trifluoromethyl-benzyl)-3,6-diisobutyl-piperazin-2-one (229)

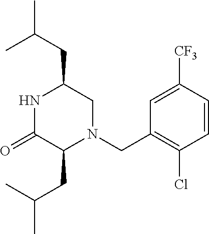

(3S,6S)-3,6-Diisobutyl-piperazine-2-one 7 (200 mg, 0.94 mmol) was on N-alkylation with 2-Bromomethyl-1-chloro-4-trifluoromethyl-benzene (254 mg, 0.94 mmol) by the method described for the preparation of compound 220 furnished the desired product 229 (60 mg, 15.78% yield) as a white solid.

¹H NMR (400 MHz, DMSO-d₆): δ 7.86 (s, 1H), 7.68-7.71 (m, 3H), 3.91 (d, J=14.4 Hz, 1H), 3.83 (d, J=14.4 Hz, 1H), 3.57-3.59 (m, 1H), 2.83-2.86 (m, 1H), 2.64-2.68 (m, 1H), 1.55-1.70 (m, 3H), 1.36-1.43 (m, 2H), 1.20-1.23 (m, 1H), 0.81-0.82 (m, 9H), 0.57-0.59 (m, 3H).

MS (ESI): m/z 405.1 [M−H]⁺

### Example 213

### (3S,6S)-3-isobutyl-4-((1R,2R)-2-phenylcyclopropanecarbonyl)-6-propylpiperazin-2-one (230)

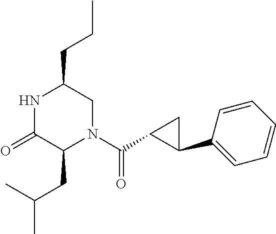

3-Isobutyl-6-propylpiperazin-2-one 16 (50 mg, 0.25 mmol) and (1R,2R)-2-phenylcyclopropanecarboxylic acid 54 (41 mg, 0.25 mmol) were coupled according to the procedure described for the preparation of compound 71 to furnish 230 (85 mg, 98% yield) as an oil.

¹H NMR (400 MHz, CDCl₃): δ 7.32-7.05 (m, 5H), 6.29 (m, 1H), 5.21-4.70 (m, 1H), 4.57-4.04 (m, 1H), 3.56-3.48 (m, 1H), 3.12-2.60 (m, 1H), 2.56-2.51 (m, 1H), 2.12-2.05 (m, 1H), 1.94-1.58 (m, 5H), 1.49-1.28 (m, 4H), 1.01-0.92 (m, 9H).

MS (ESI): m/z 343.1 (M+1)⁺

### Example 214

### (3S,6S)-6-cyclohexyl-3-isobutyl-4-((1R,2R)-2-phenylcyclopropanecarbonyl)-piperazin-2-one (231)

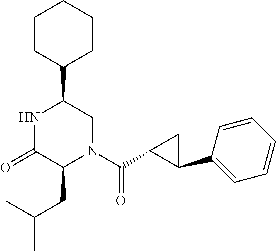

6-Cyclohexyl-3-isobutylpiperazin-2-one 18 (50 mg, 0.21 mmol) and (1R,2R)-2-phenylcyclopropanecarboxylic acid 54 (34 mg, 0.21 mmol) were coupled according to the procedure described for the preparation of compound 71 to furnish 231 (74 mg, 92% yield) as a white solid.

¹H NMR (400 MHz, CDCl₃): δ 7.36-7.05 (m, 5H), 6.29 (s, 1H), 5.20-4.68 (m, 1H), 4.57-4.03 (m, 1H), 3.36-3.28 (m, 1H), 3.21-2.69 (m, 1H), 2.55-2.50 (m, 1H), 1.94-1.88 (m, 1H), 1.84-1.42 (m, 10H), 1.41-1.23-1.04 (m, 6H), 1.01-0.92 (m, 6H).

MS (ESI): m/z 383.2 (M+1)⁺

### Example 215

### (3S,6S)-6-cyclopentyl-3-isobutyl-4-((1R,2R)-2-phenylcyclopropanecarbonyl)-piperazin-2-one (232)

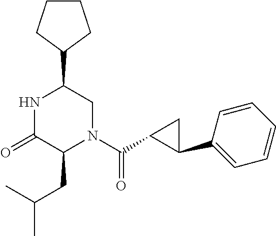

6-Cyclopentyl-3-isobutylpiperazin-2-one 17 (50 mg, 0.21 mmol) and (1R,2R)-2-phenylcyclopropanecarboxylic acid 54 (36 mg, 0.22 mmol) were coupled according to the procedure described for the preparation of compound 71 to furnish 232 (62 mg, 75% yield) as a white solid.

¹H NMR (400 MHz, CDCl₃): δ 7.32-7.05 (m, 5H), 5.85 (s, 1H), 5.21-4.71 (m, 1H), 4.57-4.07 (m, 1H), 3.36-3.26 (m, 1H), 3.14-2.63 (m, 1H), 2.55-2.50 (m, 1H), 1.93-1.57 (m, 11H), 1.33-1.23 (m, 3H), 1.02-0.92 (m, 6H).

MS (ESI): m/z 369.1 (M+1)⁺

### Example 216

### (3S,6S)-3-isobutyl-6-isopropyl-4-((1R,2R)-2-phenylcyclopropanecarbonyl)piperazin-2-one (233)

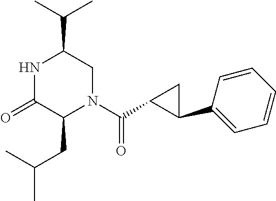

3-Isobutyl-6-isopropylpiperazin-2-one 8 (50 mg, 0.25 mmol) and (1R,2R)-2-phenylcyclopropanecarboxylic acid 54 (41 mg, 0.25 mmol) were coupled according to the procedure described for the preparation of compound 71 to furnish 233 (73 mg, 85% yield) as a white solid.

¹H NMR (400 MHz, CDCl₃): δ 7.32-7.05 (m, 5H), 5.87 (s, 1H), 5.21-4.69 (m, 1H), 4.58-4.03 (m, 1H), 3.38-3.29 (m, 1H), 3.18-2.66 (m, 1H), 2.55-2.49 (m, 1H), 1.95-1.89 (m, 1H), 1.85-1.58 (m, 5H), 1.33-1.28 (m, 1H), 1.02-0.93 (m, 12H).

MS (ESI): m/z 343.2 (M+1)⁺

### Example 217

### (3S,6S)-3-isobutyl-6-phenyl-4-((1R,2R)-2-phenylcyclopropanecarbonyl)piperazin-2-one (234)

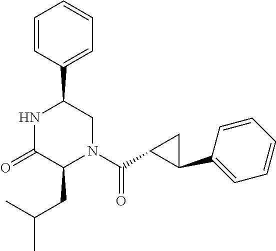

3-Isobutyl-6-phenylpiperazin-2-one 11 (50 mg, 0.22 mmol) and (1R,2R)-2-phenylcyclopropanecarboxylic acid 54 (35 mg, 0.22 mmol) were coupled according to the procedure described for the preparation of compound 71 to furnish 234 (74 mg, 91% yield) as a white solid.

¹H NMR (400 MHz, CDCl₃): δ 7.43-7.09 (m, 10H), 5.95 (s, 1H), 5.33-4.78 (m, 1H), 4.70-4.14 (m, 2H), 3.38-2.83 (m, 1H), 2.60-2.56 (m, 1H), 2.02-1.69 (m, 4H), 1.40-1.33 (m, 1H), 1.32-0.95 (m, 6H).

MS (ESI): m/z 377.1 (M+1)⁺

### Example 218

### (3S,6S)-6-cyclopropyl-3-isobutyl-4-((1R,2R)-2-phenylcyclopropanecarbonyl)-piperazin-2-one (235)

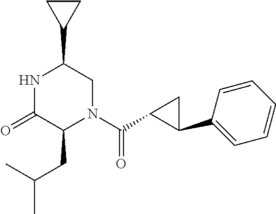

6-Cyclopropyl-3-isobutylpiperazin-2-one 12 (50 mg, 0.25 mmol) and (1R,2R)-2-phenylcyclopropanecarboxylic acid 54 (42 mg, 0.25 mmol) were coupled according to the procedure described for the preparation of compound 71 to furnish 235 (69 mg, 80% yield) as a white solid.

¹H NMR (400 MHz, CDCl₃): δ 7.32-7.04 (m, 5H), 6.00 (s, 1H), 5.20-4.76 (m, 1H), 5.56-4.14 (s, 1H), 3.31-2.79 (m, 1H), 2.72-2.60 (m, 1H), 2.57-2.48 (m, 1H), 1.93-1.73 (m, 2H), 1.68-1.59 (m, 2H), 1.33-1.26 (m, 1H), 1.03-0.94 (m, 6H), 0.79-0.74 (m, 1H), 0.61-0.55 (m, 2H), 0.39-0.20 (m, 2H).

MS (ESI): m/z 341.1 (M+1)⁺

### Example 219

### (3S,6S)-4-[(1R,2R)-2-(4-Fluoro-phenyl)-cyclopropanecarbonyl]-3,6-diisobutyl-piperazin-2-one (236)

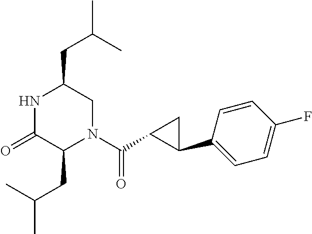

3,6-di-isobutyl-piperazin-2-one (7) substrate (60 mg, 0.28 mmol) and pure trans (R,R)[2-(p-fluoro)phenyl]-cyclopropyl-1-carboxylic acid 60 (60 mg, 0.33 mmol) were coupled according to the method described for the preparation of compound 71 to furnish product 236 (90 mg, 85% yield) as a white solid.

¹H NMR (400 MHz, CDCl₃): δ 7.09-6.93 (m, 4H), 6.36 (d, J=28.4 Hz, 1H), 5.16 (dd, J=9 & 4.4 Hz, 1H), 4.68 (dd, J=13.6 & 4 Hz, 1H), 4.53 (dd, J=9.6 & 4 Hz, 1H), 4.03 (dd, J=14.4 & 4 Hz, 1H), 3.58-3.51 (m, 1H), 3.10 (dd, J=14.4 & 11.2 Hz, 1H), 2.63-2.50 (m, 2H), 1.87-1.60 (m, 4H), 1.35-1.21 (m, 2H) and 1.01-0.90 (m, 12H).

MS (ESI): m/z 375.2 [M+1]⁺

### Example 220

### (3S,6S)-4-[(1R,2R)-2-(4-Chloro-phenyl)-cyclopropanecarbonyl]-3,6-diisobutyl-piperazin-2-one (237)

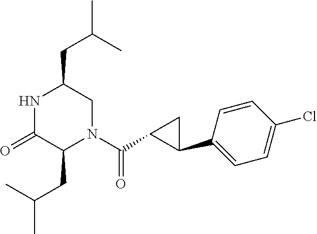

3,6-di-isobutyl-piperazin-2-one (7) substrate (80 mg, 0.35 mmol) and pure trans (R,R)[2-(p-Chloro)phenyl]-cyclopropyl-1-carboxylic acid 59 (70 mg, 0.36 mmol) were coupled according to the method described for the preparation of compound 71 to furnish product 237 (125 mg, 90.5% yield) as a white solid.

¹H NMR (400 MHz, CDCl₃): δ 7.25-7.20 (m, 2H), 7.05-6.97 (m, 2H), 6.36 (d, J=32.8 Hz, 1H), 5.15 (dd J=9.6 & 4.4 Hz, 1H), 4.68 (dd, J=13.6 & 4 Hz, 1H), 4.51 (dd, J=9.6 & 4 Hz, 1H), 4.02 (dd, J=14.4 & 4 Hz, 1H), 3.59-3.51 (m, 1H), 3.06 (dd, J=14.4 & 10.8 Hz, 1H), 2.60-2.47 (m, 2H), 1.88-1.58 (m, 4H), 1.33-1.23 (m, 2H) and 1.01-0.89 (m, 12H).

MS (ESI): m/z 391.2 [M+1]⁺

### Example 221

### (3S,6S)-4-[(1R,2R)-2-(3,4-Difluoro-phenyl)-cyclopropanecarbonyl]-3,6-diisobutyl-piperazin-2-one (238)

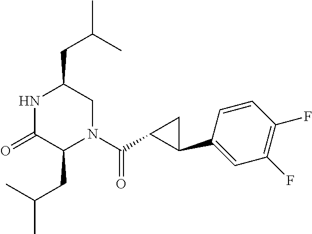

3,6-di-isobutyl-piperazin-2-one (7) substrate (60 mg, 0.28 mmol) and pure trans (R,R)[2-(3,4-di-fluoro)phenyl]-cyclopropyl-1-carboxylic acid 61 (65 mg, 0.33 mmol) were coupled according to the method described for the preparation of compound 71 to furnish product 238 (86 mg, 77.5% yield) as a white solid.

¹H NMR (400 MHz, CDCl₃): δ 7.10-7.01 (m, 1H), 6.90-6.77 (m, 2H), 6.36 (d, J=29.6 Hz, 1H), 5.16 (dd J=8.8 & 4.4 Hz, 1H), 4.67 (dd, J=13.6 & 4.4 Hz, 1H), 4.52 (dd, J=9.6 & 4 Hz, 1H), 4.02 (dd, J=14.4 & 4 Hz, 1H), 3.60-3.52 (m, 1H), 3.08 (dd, J=14.4 & 11.2 Hz, 1H), 2.60-2.47 (m, 2H), 1.87-1.60 (m, 4H), 1.34-1.22 (m, 2H) and 1.00-0.90 (m, 12H).

MS (ESI): m/z 393.1 [M+1]⁺

### Example 222

### (3S,6S)-3,6-Diisobutyl-4-[(1R,2R)-2-(4-methoxy-phenyl)-cyclopropanecarbonyl]-piperazin-2-one (239)

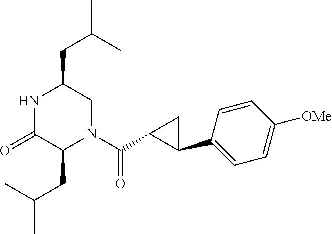

3,6-di-isobutyl-piperazin-2-one (7) substrate (60 mg, 0.28 mmol) and pure trans (R,R)[2-(p-methoxy)phenyl]-cyclopropyl-1-carboxylic acid 62 (60 mg, 0.31 mmol) were coupled according to the method described for the preparation of compound 71 to furnish product 239 (70 mg, 64% yield) as a white solid.

¹H NMR (400 MHz, CDCl₃): δ 7.05 (d, J=8.8 Hz, 1H), 6.99 (d, J=8.8 Hz, 1H), 6.82 (t, J=8.8 hz, 2H), 6.22 (d, J=19.6 Hz, 1H), 5.18 (dd J=9.6 & 4 Hz, 1H), 4.69 (dd, J=13.6 & 4 Hz, 1H), 4.03 (dd, J=13.6 & 4 Hz, 1H), 3.78 (s, 3H), 3.61-3.53 (m, 1H), 3.06 (dd, J=13.6 & 11.2 Hz, 1H), 2.57 (dd, J=13.6 & 11.2 Hz, 1H), 2.51-2.46 (m, 1H), 1.85-1.59 (m, 4H), 1.34-1.21 (m, 2H) and 1.01-0.90 (m, 12H).

MS (ESI): m/z 387.1 [M+1]⁺

### Example 223

### (3S,6S)-4-[(1R,2R)-2-(4-Fluoro-phenyl)-cyclopropanecarbonyl]-3-isobutyl-6-propyl-piperazin-2-one (240)

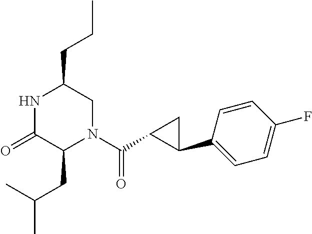

3-isobutyl-6-n-propyl-piperazin-2-one (16) substrate (40 mg, 0.20 mmol) and pure trans (R,R)[2-(p-fluoro)phenyl]-cyclopropyl-1-carboxylic acid 60 (40 mg, 0.22 mmol) were coupled according to the method described for the preparation of compound 71 to furnish product 240 (60 mg, 82.5% yield) as a white solid.

¹H NMR (400 MHz, CDCl₃): δ 7.10-6.94 (m, 4H), 5.87 (brs, 1H), 5.19 (dd J=9.6 & 4 Hz, 1H), 4.70 (dd, J=13.6 & 4 Hz, 1H), 4.55 (dd, J=9.6 & 4 Hz, 1H), 4.05 (dd, J=14 & 4 Hz, 1H), 3.55-3.49 (m, 1H), 3.11 (dd, J=14 & 11.2 Hz, 1H), 2.55-2.49 (m, 1H), 1.87-1.59 (m, 4H), 1.50-1.36 (m, 4H), and 1.02-0.93 (m, 9H).

MS (ESI): m/z 361.2 [M+1]⁺

### Example 224

### (3S,6S)-6-Cyclohexyl-4-[(1R,2R)-2-(4-fluoro-phenyl)-cyclopropanecarbonyl]-3-isobutyl-piperazin-2-one (241)

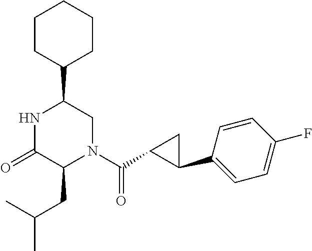

3-isobutyl-6-cyclohexyl-piperazin-2-one (18) substrate (48 mg, 0.20 mmol) and pure trans (R,R)[2-(p-fluoro)phenyl]-cyclopropyl-1-carboxylic acid 60 (40 mg, 0.22 mmol) were coupled according to the method described for the preparation of compound 71 to furnish product 241 (55 mg, 68.2% yield) as a white solid.

¹H NMR (400 MHz, CDCl₃): δ 7.10-6.94 (m, 4H), 5.84 (s, 1H), 5.19 (dd J=9.6 & 4 Hz, 1H), 4.70 (dd, J=13.6 & 4.4 Hz, 1H), 4.54 (dd, J=8.8 & 4 Hz, 1H), 4.05 (dd, J=14 & 4 Hz, 1H), 3.40-3.29 (m, 1H), 3.20 (dd, J=14.0 & 11.2 Hz, 1H), 2.76 (dd, J=13.6 & 11.2 Hz, 1H), 2.55-2.48 (m, 1H), 1.86-1.59 (m, 10H), 1.45-0.93 (m, 12H).

MS (ESI): m/z 401.2 [M+1]⁺

### Example 225

### (3S,6S)-6-Cyclopentyl-4-[(1R,2R)-2-(4-fluoro-phenyl)-cyclopropanecarbonyl]-3-isobutyl-piperazin-2-one (242)

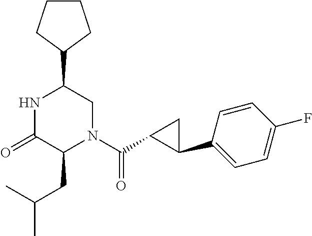

3-isobutyl-6-cyclopentyl-piperazin-2-one (17) substrate (45 mg, 0.20 mmol) and pure trans (R,R)[2-(p-fluoro)phenyl]-cyclopropyl-1-carboxylic acid 60 (40 mg, 0.22 mmol) were coupled according to the method described for the preparation of compound 71 to furnish product 242 (53 mg, 64.9% yield) as a white solid.

¹H NMR (400 MHz, CDCl₃): δ 7.10-6.94 (m, 4H), 5.80 (s, 1H), 5.20 (dd J=9.6 & 4 Hz, 1H), 4.72 (dd, J=9.6 & 4.4 Hz, 1H), 4.54 (dd, J=8.8 & 4 Hz, 1H), 4.05 (dd, J=14 & 4 Hz, 1H), 3.33-3.27 (m, 1H), 3.13 (dd, J=14.0 & 11.2 Hz, 1H), 2.67 (dd, J=13.6 & 11.2 Hz, 1H), 2.54-2.49 (m, 1H), 1.87-1.58 (m, 11H), 1.30-1.23 (m, 2H) and 1.02-0.93 (m, 6H).

MS (ESI): m/z 387.2 [M+1]⁺

### Example 226

### (3S,6S)-4-[(1R,2R)-2-(4-Chloro-phenyl)-cyclopropanecarbonyl]-6-cyclopropylmethyl-3-isobutyl-piperazin-2-one (243)

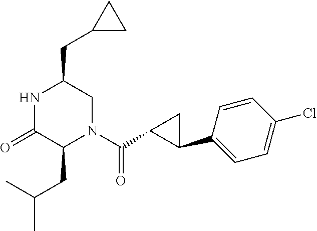

3-isobutyl-6-cyclopropylmethyl-piperazin-2-one (13) substrate (45 mg, 0.21 mmol) and pure trans (R,R)[2-(p-chloro)phenyl]-cyclopropyl-1-carboxylic acid 59 (40 mg, 0.23 mmol) were coupled according to the method described for the preparation of compound 71 to furnish product 243 (72 mg, 82.2% yield) as a white solid.

¹H NMR (400 MHz, CDCl₃): δ 7.27-7.23 (m, 2H), 7.06-6.99 (m, 2H), 6.04 (s, 1H), 5.19 (dd J=9.6 & 4 Hz, 1H), 4.75 (dd, J=13.6 & 4 Hz, 1H), 4.54 (dd, J=8.8 & 4 Hz, 1H), 4.10 (dd, J=14.4 & 4 Hz, 1H), 3.65-3.57 (m, 1H), 3.14 (dd, J=14.4 & 11.2 Hz, 1H), 2.67 (dd, J=13.6 & 11.2 Hz, 1H), 2.53-2.48 (m, 1H), 1.92-1.50 (m, 4H), 1.29-1.15 (m, 2H), 1.02-0.93 (m, 6H), 0.70-0.46 (m, 2H) and 0.20-0.07 (m, 2H).

MS (ESI): m/z 389.2 [M+1]⁺

### Example 227

### (3S,6S)-4-[(1R,2R)-2-(4-Chloro-phenyl)-cyclopropanecarbonyl]-3-isobutyl-6-propyl-piperazin-2-one (244)

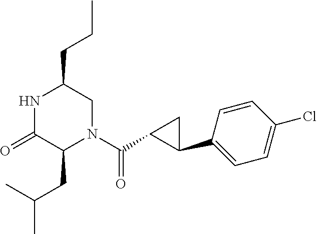

3-isobutyl-6-n-propyl-piperazin-2-one (16) substrate (40 mg, 0.20 mmol) and pure trans (R,R)[2-(p-chloro)phenyl]-cyclopropyl-1-carboxylic acid 59 (40 mg, 0.20 mmol) were coupled according to the method described for the preparation of compound 71 to furnish product 244 (45 mg, 56.2% yield) as a white solid.

¹H NMR (400 MHz, CDCl₃): δ 7.28-7.22 (m, 2H), 7.07-6.98 (m, 2H), 5.86 (s, 1H), 5.19 (dd J=9.6 & 4 Hz, 1H), 4.70 (dd, J=13.6 & 4 Hz, 1H), 4.53 (dd, J=9.6 & 4 Hz, 1H), 4.03 (dd, J=14.4 & 4 Hz, 1H), 3.55-3.46 (m, 1H), 3.10 (dd, J=14.4 & 11.2 Hz, 1H), 2.64 (dd, J=13.6 & 11.2 Hz, 1H), 2.53-2.48 (m, 1H), 1.90-1.59 (m, 4H), 1.50-1.22 (m, 3H) and 1.02-0.93 (m, 9H).

MS (ESI): m/z 377.1 [M+1]⁺

### Example 228

### (3S,6S)-4-[(1R,2R)-2-(4-Chloro-phenyl)-cyclopropanecarbonyl]-6-cyclohexyl-3-isobutyl-piperazin-2-one (245)

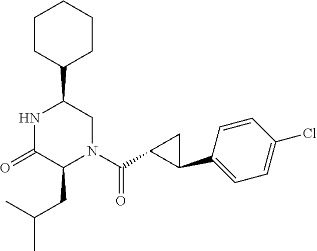

3-isobutyl-6-cyclohexyl-piperazin-2-one (18) substrate (50 mg, 0.21 mmol) and pure trans (R,R)[2-(p-chloro)phenyl]-cyclopropyl-1-carboxylic acid 59 (45 mg, 0.23 mmol) were coupled according to the method described for the preparation of compound 71 to furnish product 245 (60 mg, 65.2% yield) as a white solid.

¹H NMR (400 MHz, CDCl₃): δ 7.27-7.22 (m, 2H), 7.06-6.97 (m, 2H), 5.88 (brs, 1H), 5.18 (dd J=9.6 & 4 Hz, 1H), 4.69 (dd, J=13.6 & 4.4 Hz, 1H), 4.53 (dd, J=8.8 & 4 Hz, 1H), 4.02 (dd, J=14 & 4 Hz, 1H), 3.37-3.28 (m, 1H), 3.20 (dd, J=14.0 & 11.2 Hz, 1H), 2.73 (dd, J=13.6 & 11.2 Hz, 1H), 2.53-2.47 (m, 1H), 1.91-1.60 (m, 10H), 1.43-0.93 (m, 10H).

MS (ESI): m/z 417.2 [M+1]⁺

### Example 229

### (3S,6S)-4-[(1R,2R)-2-(4-Chloro-phenyl)-cyclopropanecarbonyl]-6-cyclopentyl-3-isobutyl-piperazin-2-one (246)

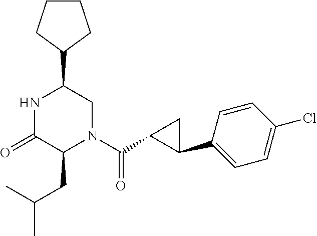

3-isobutyl-6-cyclopentyl-piperazin-2-one (17) substrate (40 mg, 0.20 mmol) and pure trans (R,R)[2-(p-chloro)phenyl]-cyclopropyl-1-carboxylic acid 59 (40 mg, 0.20 mmol) were coupled according to the method described for the preparation of compound 71 to furnish product 246 (40 mg, 49.5% yield) as a white solid.

¹H NMR (400 MHz, CDCl₃): δ 7.27-7.23 (m, 2H), 7.06-6.98 (m, 2H), 5.79 (brs, 1H), 5.21 (dd J=9.6 & 4 Hz, 1H), 4.72 (dd, J=14 & 4.4 Hz, 1H), 4.53 (dd, J=8.8 & 4 Hz, 1H), 4.04 (dd, J=14.0 & 4.0 Hz, 1H), 3.34-3.26 (m, 1H), 3.13 (dd, J=14.0 & 11.2 Hz, 1H), 2.67 (dd, J=13.6 & 11.2 Hz, 1H), 2.54-2.47 (m, 1H), 1.91-1.58 (m, 10H), 1.29-1.24 (m, 2H) and 1.02-0.93 (m, 6H).

MS (ESI): m/z 403.5 [M+1]⁺

### Example 230

### (3S,6S)-4-[(1R,2R)-2-(4-Fluoro-phenyl)-cyclopropanecarbonyl]-3-isobutyl-6-phenyl-piperazin-2-one (247)

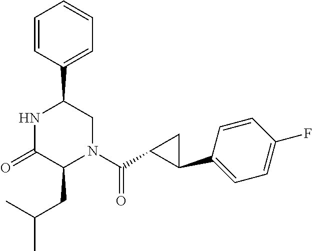

3-isobutyl-6-phenyl-piperazin-2-one (11) substrate (46 mg, 0.20 mmol) and pure trans (R,R)[2-(p-fluoro)phenyl]-cyclopropyl-1-carboxylic acid 60 (37 mg, 0.21 mmol) were coupled according to the method described for the preparation of compound 71 to furnish product 247 (55 mg, 70.4% yield) as a white solid.

¹H NMR (400 MHz, CDCl₃): δ 7.41-7.32 (m, 5H), 7.08-6.93 (m, 4H), 6.05 (d, J=6 Hz, 1H), 5.30 (dd, J=10.0 & 4.0 Hz, 1H), 4.79 (dd, J=13.6 & 4.4 Hz, 1H), 4.69-4.62 (m, 1H), 4.15 (dd, J=14.4 & 4.0 Hz, 1H), 3.36 (dd, J=14.4 & 11.2 Hz, 1H), 2.62-2.53 (m, 1H), 1.94-1.66 (m, 4H), 1.34-1.24 (m, 1H) and 1.04-0.95 (m, 6H).

MS (ESI): m/z 395.1 [M+1]⁺

### Example 231

### (3S,6S)-4-[(1R,2R)-2-(4-Chloro-phenyl)-cyclopropanecarbonyl]-3-isobutyl-6-phenyl-piperazin-2-one (248)

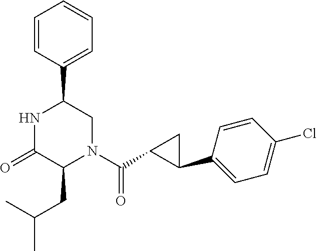

3-isobutyl-6-phenyl-piperazin-2-one (11) substrate (46 mg, 0.20 mmol) and pure trans (R,R)[2-(p-chloro)phenyl]-cyclopropyl-1-carboxylic acid 59 (40 mg, 0.20 mmol) were coupled according to the method described for the preparation of compound 71 to furnish product 248 (64 mg, 78.7% yield) as a white solid.

¹H NMR (400 MHz, CDCl₃): δ 7.42-7.22 (m, 7H), 7.04-7.01 (m, 2H), 5.96 (d, J=8 Hz, 1H), 5.30 (dd, J=10.0 & 4.0 Hz, 1H), 4.78 (dd, J=13.6 & 4.4 Hz, 1H), 4.69-4.61 (m, 1H), 4.14 (dd, J=14.4 & 4.0 Hz, 1H), 3.36 (dd, J=14.4 & 11.2 Hz, 1H), 2.86 (dd, J=13.6 & 11.6 Hz, 1H), 2.59-2.52 (m, 1H), 1.96-1.68 (m, 4H), 1.38-1.24 (m, 1H) and 1.04-0.96 (m, 6H).

MS (ESI): m/z 411.2 [M+1]⁺

### Example 232

### (3S,6S)-4-[(1R,2R)-2-(4-tert-Butyl-phenyl)-cyclopropanecarbonyl]-3,6-diisobutyl-piperazin-2-one (249)

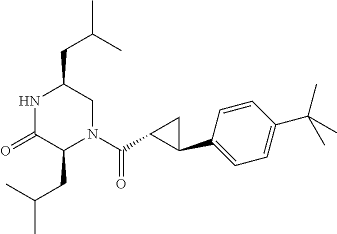

3,6-di-isobutyl-piperazin-2-one (7) substrate (42 mg, 0.20 mmol) and pure trans (R,R)[2-(p-tert-butyl)phenyl]-cyclopropyl-1-carboxylic acid 65 (45 mg, 0.21 mmol) were coupled according to the method described for the preparation of compound 71 to furnish product 249 (60 mg, 73.5% yield) as a white solid.

¹H NMR (400 MHz, CDCl₃): δ 7.33 (d, J=8.4 Hz, 1H), 7.29 (d, J=8.4 Hz, 1H), 7.07 (d, J=8.4 Hz, 1H), 6.01 (d, J=8.4 Hz, 1H), 5.78 (s, 1H), 5.20 (dd J=9.6 & 4 Hz, 1H), 4.70 (dd, J=13.6 & 4 Hz, 1H), 4.57 (dd, J=8.8 & 4 Hz, 1H), 4.06 (dd, J=13.6 & 4 Hz, 1H), 3.61-3.52 (m, 1H), 3.06 (dd, J=14.0 & 11.2 Hz, 1H), 2.60 (dd, J=13.6 & 11.2 Hz, 1H), 2.54-2.49 (m, 1H), 1.92-1.61 (m, 4H), 1.34-1.26 (m, 10H) and 1.02-0.91 (m, 12H).

MS (ESI): m/z 413.3 [M+1]⁺

### Example 233

### (3S,6S)-4-[(1R,2R)-2-(4-Chloro-2-fluoro-phenyl)-cyclopropanecarbonyl]-3,6-diisobutyl-piperazin-2-one (250)

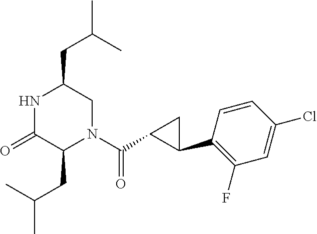

3,6-di-isobutyl-piperazin-2-one (7) substrate (42 mg, 0.20 mmol) and pure trans (R,R)[2-(2-fluoro-4-chloro)phenyl]-cyclopropyl-1-carboxylic acid 63 (45 mg, 0.21 mmol) were coupled according to the method described for the preparation of compound 71 to furnish product 250 (10 mg, 11.1% yield) as a white solid.

¹H NMR (400 MHz, CDCl₃): δ 7.08-6.89 (m, 3H), 5.86 (s, 1H), 5.20 (dd J=9.6 & 4 Hz, 1H), 4.69 (dd, J=13.6 & 4 Hz, 1H), 4.56 (dd, J=8.8 & 4.0 Hz, 1H), 4.04 (dd, J=14.0 & 4.0 Hz, 1H), 3.61-3.54 (m, 1H), 3.11 (dd, J=14.0 & 11.2 Hz, 1H), 2.60 (dd, J=13.6 & 11.2 Hz, 1H), 2.62-2.52 (m, 2H), 1.99-1.63 (m, 4H), 1.35-1.30 (m, 10H) and 1.02-0.92 (m, 12H).

MS (ESI): m/z 409.2 [M+1]⁺

### Example 234

### (3S,6S)-4-[(1R,2R)-2-(4-Chloro-2-fluoro-phenyl)-cyclopropanecarbonyl]-6-ethyl-3-isobutyl-piperazin-2-one (251)

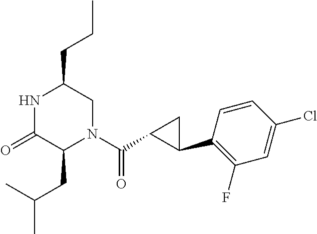

3-isobutyl-6-n-propyl-piperazin-2-one (16) substrate (40 mg, 0.20 mmol) and pure trans (R,R)[2-(2-fluoro-4-chloro)phenyl]-cyclopropyl-1-carboxylic acid 63 (45 mg, 0.21 mmol) were coupled according to the method described for the preparation of compound 71 to furnish product 251 (45 mg, 56.5% yield) as a white solid.

¹H NMR (400 MHz, CDCl₃): δ 7.08-6.89 (m, 3H), 5.94 (d, J=15.8 Hz, 1H), 5.21-5.18 (m, 1H), 4.72-4.68 (m, 1H), 4.56 (dd, J=8.4 & 4 Hz, 1H), 4.06 (dd, J=14.4 & 4 Hz, 1H), 3.55-3.48 (m, 1H), 3.13 (dd, J=14.4 & 11.2 Hz, 1H), 2.67-2.52 (m, 2H), 1.98-1.64 (m, 4H), 1.47-1.30 (m, 3H) and 1.02-0.94 (m, 9H).

MS (ESI): m/z 395.2 [M+1]⁺

### Example 235

### (3S,6S)-4-[(1R,2R)-2-(4-Chloro-2-fluoro-phenyl)-cyclopropanecarbonyl]-3-isobutyl-6-(1-propyl-butyl)-piperazin-2-one (252)

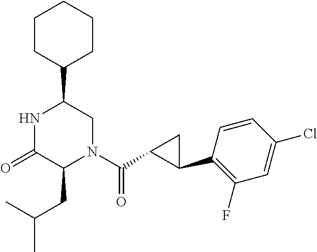

3-isobutyl-6-cyclohexyl-piperazin-2-one (18) substrate (48 mg, 0.20 mmol) and pure trans (R,R)[2-(2-fluoro-4-chloro)phenyl]-cyclopropyl-1-carboxylic acid 63 (45 mg, 0.21 mmol) were coupled according to the method described for the preparation of compound 71 to furnish product 252 (40 mg, 45.7% yield) as a white solid.

¹H NMR (400 MHz, CDCl₃): δ 7.08-6.89 (m, 3H), 5.95 (brs, 1H), 5.18 (dd J=9.6 & 4 Hz, 1H), 4.69 (dd, J=13.6 & 4.4 Hz, 1H), 4.54 (dd, J=9.6 & 4.0 Hz, 1H), 4.05 (dd, J=9.6 & 4.0 Hz, 1H), 3.37-3.30 (m, 1H), 3.23 (dd, J=14.0 & 11.2 Hz, 1H), 2.73 (dd, J=13.6 & 11.2 Hz, 1H), 2.59-2.51 (m, 1H), 1.98-1.62 (m, 11H), 1.42-0.93 (m, 11H).

MS (ESI): m/z 435.2 [M+1]⁺

### Example 236

### (3S,6S)-4-[(1R,2R)-2-(4-Chloro-2-fluoro-phenyl)-cyclopropanecarbonyl]-6-cyclopentyl-3-isobutyl-piperazin-2-one (253)

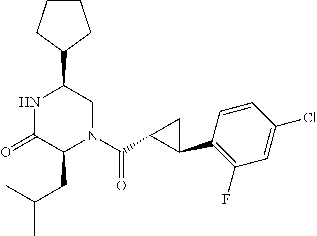

3-isobutyl-6-cyclopentyl-piperazin-2-one (17) substrate (45 mg, 0.20 mmol) and pure trans (R,R)[2-(2-fluoro-4-chloro)phenyl]-cyclopropyl-1-carboxylic acid 63 (45 mg, 0.21 mmol) were coupled according to the method described for the preparation of compound 71 to furnish product 253 (40 mg, 47.4% yield) as a white solid.

¹H NMR (400 MHz, CDCl₃): δ 7.08-6.89 (m, 3H), 5.91 (brs, 1H), 5.18 (dd J=9.6 & 4 Hz, 1H), 4.72 (dd, J=13.6 & 4.4 Hz, 1H), 4.54 (dd, J=9.6 & 4.0 Hz, 1H), 4.07 (dd, J=15.2 & 4.0 Hz, 1H), 3.35-3.28 (m, 1H), 3.15 (dd, J=14.0 & 11.2 Hz, 1H), 2.66 (dd, J=13.6 & 11.2 Hz, 1H), 2.59-2.51 (m, 1H), 1.99-1.57 (m, 12H), 1.33-1.19 (m, 1H) and 1.02-0.93 (m, 6H).

MS (ESI): m/z 395.1 [M+1]⁺

### Example 237

### (3S,6S)-4-[(1R,2R)-2-(4-Chloro-2-fluoro-phenyl)-cyclopropanecarbonyl]-3-isobutyl-6-isopropyl-piperazin-2-one (254)

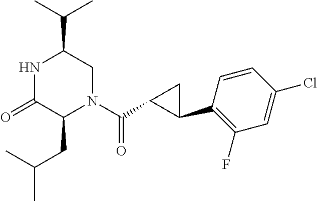

3-isobutyl-6-isopropyl-piperazin-2-one (8) substrate (20 mg, 0.10 mmol) and pure trans (R,R)[2-(2-fluoro-4-chloro)phenyl]-cyclopropyl-1-carboxylic acid 63 (20 mg, 0.10 mmol) were coupled according to the method described for the preparation of compound 71 to furnish product 254 (8 mg, 18.7% yield) as a white solid.

¹H NMR (400 MHz, CDCl₃): δ 7.08-6.89 (m, 3H), 5.77 (brs, 1H), 5.18 (dd J=9.6 & 4 Hz, 1H), 4.72 (dd, J=13.6 & 4.4 Hz, 1H), 4.55 (dd, J=9.6 & 4.0 Hz, 1H), 4.05 (dd, J=9.6 & 4.0 Hz, 1H), 3.37-3.31 (m, 1H), 3.20 (dd, J=14.0 & 11.2 Hz, 1H), 2.66 (dd, J=13.6 & 11.2 Hz, 1H), 2.60-2.51 (m, 1H), 1.99-1.63 (m, 3H), 1.35-1.29 (m, 1H) and 1.02-0.94 (m, 12H).

MS (ESI): m/z 395.1 [M+1]⁺

### Example 238

### (3S,6S)-4-[(1R,2R)-2-(4-Chloro-2-fluoro-phenyl)-cyclopropanecarbonyl]-3-isobutyl-6-phenyl-piperazin-2-one (255)

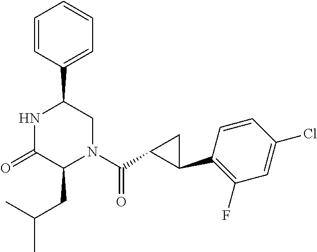

3-isobutyl-6-phenyl-piperazin-2-one (11) substrate (23 mg, 0.10 mmol) and pure trans (R,R)[2-(2-fluoro-4-chloro)phenyl]-cyclopropyl-1-carboxylic acid 63 (20 mg, 0.10 mmol) were coupled according to the method described for the preparation of compound 71 to furnish product 255 (36 mg, 76.3% yield) as a white solid.

¹H NMR (400 MHz, CDCl₃): δ 7.43-7.32 (m, 5H), 7.08-6.92 (m, 3H), 6.08 (d, J+8 Hz, 1H), 5.31-5.28 (m, 1H), 4.80-4.76 (m, 1H), 4.68-4.63 (m, 1H), 4.14 (dd, J=14.8 & 4.0 Hz, 1H), 3.38 (dd, J=14.4 & 4.0 Hz, 1H), 2.86 (dd, J=13.6 & 11.2 Hz, 1H), 2.65-2.54 (m, 1H), 2.03-1.68 (m, 3H), 1.41-1.32 (m, 1H) and 1.04-0.96 (m, 6H).

MS (ESI): m/z 429.0 [M+1]⁺

### Example 239

### (3S,6S)-4-[(1R,2R)-2-(2,4-Difluoro-phenyl)-cyclopropanecarbonyl]-3,6-diisobutyl-piperazin-2-one (256)

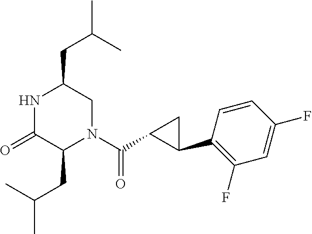

3,6-di-isobutyl-piperazin-2-one (7) substrate (42 mg, 0.20 mmol) and pure trans (R,R)[2-(2,4-di-fluoro)phenyl]-cyclopropyl-1-carboxylic acid 64 (40 mg, 0.20 mmol) were coupled according to the method described for the preparation of compound 71 to furnish product 256 (59 mg, 68.3% yield) as a white solid.

¹H NMR (400 MHz, CDCl₃): δ 7.07-6.93 (m, 1H), 6.83-6.75 (m, 2H), 5.78 (s, 1H), 5.21 (dd J=9.6 & 4 Hz, 1H), 4.70 (dd, J=13.6 & 4 Hz, 1H), 4.57 (dd, J=8.8 & 4.0 Hz, 1H), 4.06 (dd, J=14.4 & 4.0 Hz, 1H), 3.61-3.55 (m, 1H), 3.13 (dd, J=14.4 & 11.2 Hz, 1H), 2.66-2.51 (m, 2H), 1.96-1.64 (m, 2H), 1.99-1.63 (m, 4H), 1.35-1.30 (m, 2H) and 1.02-0.92 (m, 12H).

MS (ESI): m/z 393.2 [M+1]⁺

### Example 240

### (3S,6S)-4-[(1R,2R)-2-(3,4-Difluoro-phenyl)-cyclopropanecarbonyl]-3-isobutyl-6-propyl-piperazin-2-one (257)

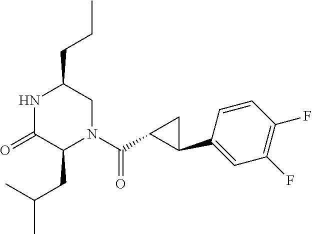

3-isobutyl-6-propyl-piperazin-2-one (16) substrate (20 mg, 0.10 mmol) and pure trans (R,R)[2-(3,4-di-fluoro)phenyl]-cyclopropyl-1-carboxylic acid 61 (20 mg, 0.10 mmol) were coupled according to the method described for the preparation of compound 71 to furnish product 257 (8 mg, 20% yield) as a white solid.

¹H NMR (400 MHz, CDCl₃): δ 7.10-7.03 (m, 1H), 6.90-6.78 (m, 2H), 5.81 (d, J=6.4 Hz, 1H), 5.19 (dd J=8.8 & 4.4 Hz, 1H), 4.70 (dd, J=13.6 & 4.0 Hz, 1H), 4.53 (dd, J=9.6 & 4 Hz, 1H), 4.02 (dd, J=14.4 & 4 Hz, 1H), 3.60-3.49 (m, 1H), 3.13 (dd, J=14.0 & 11.2 Hz, 1H), 2.64 (dd, J=14.0 & 11.2 Hz, 1H), 2.54-2.47 (m, 1H), 1.88-1.57 (m, 4H), 1.48-1.38 (m, 2H), 1.27-1.23 (m, 1H) and 1.02-0.94 (m, 6H).

MS (ESI): m/z 379.1 [M+1]⁺

### Example 241

### (3S,6S)-6-Cyclohexyl-4-[(1R,2R)-2-(3,4-difluoro-phenyl)-cyclopropanecarbonyl]-3-isobutyl-piperazin-2-one (258)

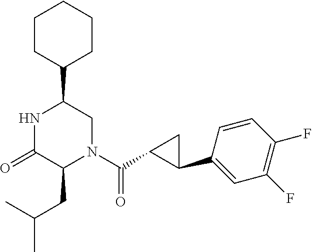

3-isobutyl-6-cyclohexyl-piperazin-2-one (18) substrate (24 mg, 0.10 mmol) and pure trans (R,R)[2-(3,4-di-fluoro)phenyl]-cyclopropyl-1-carboxylic acid 61 (20 mg, 0.10 mmol) were coupled according to the method described for the preparation of compound 71 to furnish product 258 (15 mg, 35.6% yield) as a white solid.

¹H NMR (400 MHz, CDCl₃): δ 7.09-7.03 (m, 1H), 6.89-6.77 (m, 2H), 5.87 (s, 1H), 5.19 (dd J=8.8 & 4.4 Hz, 1H), 4.70 (dd, J=13.6 & 4.4 Hz, 1H), 4.53 (dd, J=8.8 & 4 Hz, 1H), 4.02 (dd, J=14.4 & 4 Hz, 1H), 3.38-3.18 (m, 2H), 2.74 (dd, J=13.6 & 11.2 Hz, 1H), 2.54-2.47 (m, 1H), 1.88-1.61 (m, 10H), 1.46-0.93 (m, 10H).

MS (ESI): m/z 419.2 [M+1]⁺

### Example 242

### (3S,6S)-6-Cyclopentyl-4-[(1R,2R)-2-(3,4-difluoro-phenyl)-cyclopropanecarbonyl]-3-isobutyl-piperazin-2-one (259)

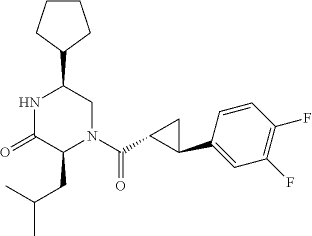

3-Isobutyl-6-cyclopentyl-piperazin-2-one (17) substrate (23 mg, 0.10 mmol) and pure trans (R,R)[2-(3,4-di-fluoro)phenyl]-cyclopropyl-1-carboxylic acid 61 (20 mg, 0.10 mmol) were coupled according to the method described for the preparation of compound 71 to furnish product 259 (32 mg, 71.8% yield) as a white solid.

¹H NMR (400 MHz, CDCl₃): δ 7.10-7.02 (m, 1H), 6.92-6.77 (m, 2H), 5.98 (s, 1H), 5.18 (dd J=9.6 & 4.0 Hz, 1H), 4.71 (dd, J=13.6 & 4.4 Hz, 1H), 4.52 (dd, J=8.8 & 4 Hz, 1H), 4.02 (dd, J=14.4 & 4 Hz, 1H), 3.34-3.26 (m, 1H), 3.14 (dd, J=14.0 & 11.2 Hz, 1H). 2.66 (dd, J=13.6 & 11.2 Hz, 1H), 2.53-2.46 (m, 1H), 1.88-1.59 (m, 10H), 1.26-1.20 (m, 2H) and 1.01-0.93 (m, 6H).

MS (ESI): m/z 405.1 [M+1]⁺

### Example 243

### (3S,6S)-4-[(1R,2R)-2-(3,4-Difluoro-phenyl)-cyclopropanecarbonyl]-3-isobutyl-6-phenyl-piperazin-2-one (260)

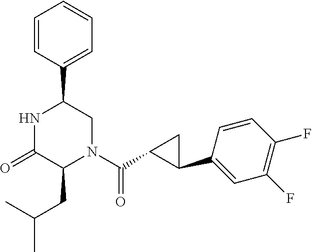

3-Isobutyl-6-phenyl-piperazin-2-one (11) substrate (20 mg, 0.10 mmol) and pure trans (R,R)[2-(3,4-di-fluoro)phenyl]-cyclopropyl-1-carboxylic acid 61 (20 mg, 0.10 mmol) were coupled according to the method described for the preparation of compound 71 to furnish product 260 (23 mg, 56.3% yield) as a white solid.

¹H NMR (400 MHz, CDCl₃): δ 7.44-7.32 (m, 5H), 7.12-7.03 (m, 1H), 6.90-6.81 (m, 2H), 5.99 (d, J=7.6 Hz, 1H), 5.30 (dd, J=10.0 & 4.0 Hz, 1H), 4.79 (dd, J=13.6 & 4.4 Hz, 1H), 4.69-4.61 (m, 1H), 4.13 (dd, J=14.4 & 4.0 Hz, 1H), 3.38 (dd, J=14.4 & 11.2 Hz, 1H), 2.87 (dd, J=13.6 & 11.2 Hz, 1H), 2.58-2.52 (m, 1H), 1.94-1.67 (m, 3H), 1.34-1.24 (m, 1H) and 1.04-0.96 (m, 6H).

MS (ESI): m/z 413.1 [M+1]⁺

### Example 244

### (3S,6S)-4-((E)-3-(4-(dimethylamino)phenyl)acryloyl)-3,6-diisobutylpiperazin-2-one (261)

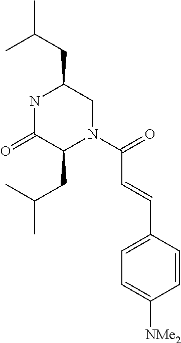

Compound 7 (60 mg, 0.28 mmol) and (E)-3-[4-(dimethylamino)phenyl]acrylic acid (60 mg, 0.3 mmol) were coupled according to the method described for compound 70 to give the product 261 (100 mg, 96% yield) as an yellow syrup.

¹H NMR (400 MHz, CDCl₃): δ 7.69 (d, J=16.0 Hz, 1H), 7.41-7.39 (brd, 2H), 6.68-6.66 (brd, 2H), 6.64-6.58 (m, 1H), 5.83 (brs, 1H), 4.83-4.80 (m, 1H), 4.61-4.59 (m, 1H), 3.66-3.57 (m, 1H), 3.01 (s, 6H); 2.68-2.62 (m, 1H), 1.86-1.66 (m, 4H), 1.38-1.32 (m, 2H), 1.05-0.92 (m, 12H).

MS (ESI): m/z 387.4 [M+H]⁺.

### Example 245

### (3S,6S)-3,6-diisobutyl-4-(3-(pyridin-3-yl)propioloyl)piperazin-2-one (262)

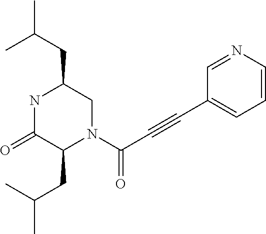

Compound 7 (60 mg, 0.28 mmol) and 3-(pyridin-3-yl)propiolic acid (60 mg, 0.41 mmol) were coupled according to the method described for compound 70 to give the product 262 (90 mg, 93% yield) as a white solid.

¹H NMR (400 MHz, CDCl₃): δ 8.63 (dd, J=8.0 & 4.0 Hz, 2H), 7.64 (dd, J=16.0 & 4.0 Hz, 1H), 7.37-7.33 (m, 2H), 6.99 (dd, J=16.0 & 6.8 Hz, 1H), 5.85 (s, 1H), 5.35-5.32 (m, 0.6H), 4.87 (d, J=2.8 Hz, 1H), 4.80 (m, 1H), 4.53 (brd, 1H), 3.70-3.52 (m, 1H), 2.71 (dd, J=16.0 & 11.2 Hz, 1H), 1.88-1.66 (m, 6H), 1.40-1.35 (m, 2H), 1.06-0.91 (m, 12H)

MS (ESI): m/z 342.2 [M+H]⁺

### Example 246

### (3S,6S)-3,6-diisobutyl-4-((E)-3-(4-methoxyphenyl)acryloyl)piperazin-2-one (263)

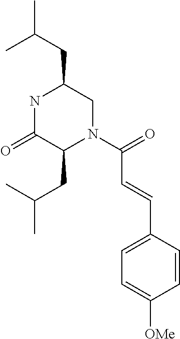

Compound 7 (60 mg, 0.29 mmol) and (E)-3-(4-methoxyphenyl)acrylic acid (60 mg, 0.34 mmol) were coupled according to the method described for compound 70 to give the product 263 (84 mg, 96% yield) as a white solid.

¹H NMR (400 MHz, CDCl₃): δ 7.71 (m, 1H), 7.46-7.43 (brd, 2H), 6.92-6.90 (brd, 2H), 6.69 (m, 1H), 5.93 (brs, 1H), 4.79 (m, 1H), 4.56 (m, 1H), 3.83 (s, 3H) 3.66-3.52 (m, 1H), 2.67 (m, 1H), 1.87-1.60 (m, 5H), 1.38-1.28 (m, 2H), 1.05-0.93 (m, 12H).

MS (ESI): m/z 373.2 [M+H]⁺.

### Example 247

### (3S,6S)-4-[3-(3-chlorophenyl)-acryloyl]-3,6-diisobutyl-piperazin-2-one (264)

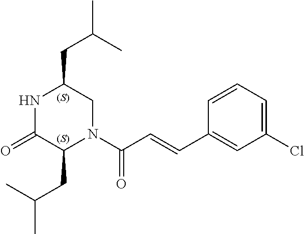

3,6-diisobutyl-piperazin-2-one (7) substrate (60 mg, 0.28 mmol) and 3-chloro-cinnamic acid (60 mg, 0.33 mmol) were coupled according to the procedure described for compound 70 to furnish 264 (88 mg, 98% yield) as a white solid.

¹H NMR (400 MHz, CDCl₃): δ 7.67 (dd, J=15.6 & 5.6 Hz, 1H), 7.50 (d, J=15.6 Hz, 1H); 7.38-7.29 (m, 2H), 6.83 (dd, J=15.6 & 5.6 Hz, 1H), 5.95 (brs, 1H), 5.33 (dd, J=9.6 & 4.0 Hz, 1H), 4.79 (dd, J=13.2 & 4.0 Hz, 1H), 4.56-4.53 (m, 1H), 3.65-3.56 (m, 2H), 2.68 (dd, J=13.2 & 11.2 Hz, 1H), 1.88-1.63 (m, 4H), 1.39-1.34 (m, 2H), 1.05-0.93 (m, 12H).

MS (ESI): m/z 379 [M+H]⁺

### Example 248

### (3S,6S)-4-[3-(2-chlorophenyl)-acryloyl]-3,6-diisobutyl-piperazin-2-one (265)

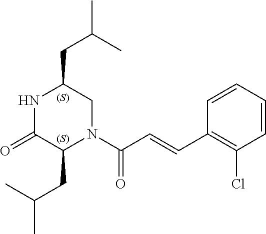

Compound 7 (60 mg, 0.28 mmol) and 2-chloro-cinnamic acid (60 mg, 0.33 mmol) were coupled according to the procedure described for compound 70 to furnish 265 (50 mg, 56% yield) as a white solid.

¹H NMR (400 MHz, CDCl₃): δ 7.84 (d, J=15.6, 1H), 7.50 (d, J=15.6 hz, 1H); 7.35 (dd, J=8.4 & 2.0 Hz, 1H), 7.20-7.16 (m, 1H), 6.99 (d, J=15.6 Hz, 1H); 5.89 (brs, 1H), 5.33 (dd, J=8.0 & 4.0 Hz, 1H), 4.79 (dd, J=13.2 & 4.0 Hz, 1H), 4.46 (dd, J=8.0 & 4.0 Hz, 1H), 3.69-3.56 (m, 2H), 2.71 (dd, J=13.2 & 11.2 Hz, 1H), 1.84-1.64 (m, 4H), 1.39-1.32 (m, 2H), 1.07-0.92 (m, 12H).

MS (ESI): m/z 379 [M+H]⁺

### Example 249

### (3S,6S)-4-[3-(2-chlorophenyl)-acryloyl]-3,6-diisobutyl-piperazin-2-one (266)

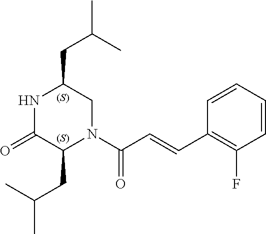

Compound 7 (60 mg, 0.28 mmol) and 2-fluoro-cinnamic acid (70 mg, 0.42 mmol) were coupled according to the procedure described for compound 70 to furnish 266 (100 mg, 96% yield) as a white solid.

¹H NMR (400 MHz, CDCl₃): δ 7.76 (t, J=15.2, 1H), 7.45 (t, J=7.2 hz, 1H); 7.35-7.30 (m, 1H), 7.17-6.97 (m, 3H), 6.28 (brs, 1H), 5.32 (dd, J=9.6 & 4.0 Hz, 1H), 4.79 (dd, J=13.2 & 4.0 Hz, 1H), 4.54 (dd, J=8.0 & 4.0 Hz, 1H), 3.64-3.54 (m, 2H), 2.67 (dd, J=13.2 & 11.2 Hz, 1H), 1.88-1.64 (m, 4H), 1.39-1.32 (m, 2H), 1.07-0.92 (m, 12H).

MS (ESI): m/z 361.2 [M+H]⁺

### Example 250

### (3S,6S)-4-[3-(2,4-dichloro-phenyl)-acryloyl]-3,6-diisobutyl-piperazin-2-one (267)

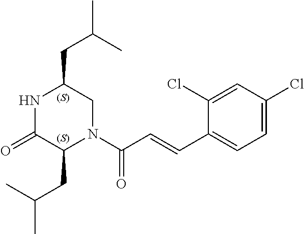

Compound 7 (53 mg, 0.25 mmol) and 3-(4-Chloro-2-fluoro-phenyl)-acrylic acid (60 mg, 0.28 mmol) were coupled according to the procedure described for compound 70 to furnish 267 (95 mg, 97% yield) as a white solid.

¹H NMR (400 MHz, CDCl₃): δ 7.99 (dd, J=24.4 & 15.2 Hz, 1H), 7.51-7.44 (m, 2H), 7.28 (m, 1H), 6.82 (dd, J=15.2 & 8.0 Hz, 1H), 5.78 (s, 1H), 5.33 (m, 1H), 4.79 (m, 1H), 4.51 (m, 1H), 3.66-3.56 (m, 2H), 2.68 (dd, J=13.6 & 11.2 Hz, 1H), 1.87-1.66 (m, 4H), 1.40-1.34 (m, 2H), 1.06-0.93 (m, 12H).

MS (ESI): m/z 413 [M+H]⁺

### Example 251

### (3S,6S)-3,6-diisobutyl-4-((E)-3-(4-methylsulfanyl-phenyl)acryloyl)piperazin-2-one (268)

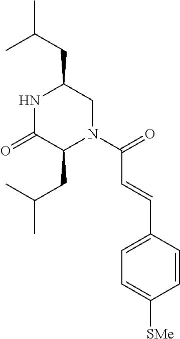

Compound 7 (55 mg, 0.26 mmol) and (E)-3-(4-methylsulfanylphenyl)acrylic acid (60 mg, 0.34 mmol) were coupled according to the procedure described for compound 70 to furnish 268 (84 mg, 96% yield) as a pale green solid.

¹H NMR (400 MHz, CDCl₃): δ 7.68 (dd, J=15.2 & 8.0 Hz, 1H), 7.42 (t, J=8.0 Hz, 2H), 7.22 (dd, J=8.0 & 1.6 Hz, 1H), 6.78 (dd, J=15.2 & 8.0 Hz, 1H), 5.90 (brs, 1H), 5.34 (dd, J=9.6 & 4.0 Hz, 1H), 4.79 (dd, J=13.6 & 4.0 Hz, 1H), 4.56 (dd, J=8.0 & 4.0 Hz, 1H), 3.65-3.56 (m, 1H), 2.67 (m, 1H), 2.50 (s, 3H), 1.90-1.63 (m, 4H), 1.38-1.20 (m, 2H), 1.05-0.93 (m, 12H).

MS (ESI): m/z 389.5 [M+H]⁺.

### Example 252

### (3S,6S)-3,6-diisobutyl-4-((E)-3-(4-tert-butyl-phenyl)acryloyl)piperazin-2-one (269)

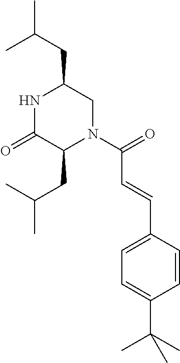

Compound 7 (55 mg, 0.26 mmol) and (E)-3-(4-tert-butylphenyl)acrylic acid (60 mg, 0.28 mmol) were coupled according to the procedure described for compound 70 to furnish 269 (87 mg, 85% yield) as a white solid.

¹H NMR (400 MHz, CDCl₃): δ 7.73 (d, J=15.2 Hz, 1H), 7.46-7.39 (m, 4H), 6.79 (dd, J=15.2 Hz, 1H), 5.93 (brs, 1H), 5.34 (dd, J=8.0 & 4.0 Hz, 1H), 4.81 (dd, J=13.2 & 4.0 Hz, 1H), 4.57 (dd, J=8.0 & 4.0 Hz, 1H), 3.65-3.55 (m, 1H), 2.67 (dd, J=13.2 & 11.2 Hz, 1H), 1.88-1.63 (m, 4H), 1.38-1.30 (m, 11H), 1.05-0.93 (m, 12H).

MS (ESI): m/z 399.6 [M+H]⁺.

### Example 253

### Methyl 4-((E)-3-((2S,5S)-2,5-diisobutyl-3-oxopiperazin-1-yl)-3-oxoprop-1-en-1-yl)-benzoate (270)

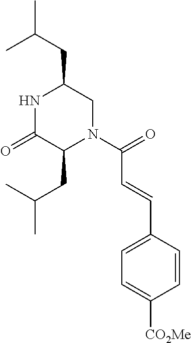

Compound 7 (55 mg, 0.26 mmol) and (E)-3-[4-(methoxycarbonyl)phenyl]acrylic acid (60 mg, 0.29 mmol) were coupled according to the procedure described for compound 70 to furnish 270 (94 mg, 90% yield) as a white solid.

¹H NMR (400 MHz, CDCl₃): δ 8.03 (d, J=8 Hz, 2H), 7.72 (dd, J=15.2 Hz, 1H), 7.55 (t, J=8.0 Hz, 2H), 6.91 (dd, J=15.2 & 8.0 Hz, 1H), 6.30 (brs, 1H), 5.30 (dd, J=8.0 & 4.0 Hz, 1H), 4.78 (dd, J=13.2 & 4.0 Hz, 1H), 4.53 (t, J=8.0 Hz, 1H), 3.91 (s, 1H), 3.65-3.55 (m, 1H), 2.67 (t, J=12.0 Hz, 1H), 1.89-1.63 (m, 4H), 1.38-1.34 (m, 2H), 1.04-0.93 (m, 12H).

MS (ESI): m/z 401.5 [M+H]⁺.

### Example 254

### (3S,6S)-4-[3-(2,6-Difluoro-phenyl)-acryloyl]-3,6-diisobutyl-piperazin-2-one (271)

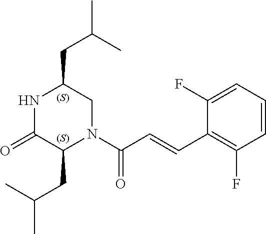

Compound 7 (65 mg, 0.30 mmol) and 3-(2,4-Difluoro-phenyl)-acrylic acid (60 mg, 0.32 mmol) were coupled according to the procedure described for compound 70 to furnish 271 (90 mg, 74% yield) as a white solid.

¹H NMR (400 MHz, CDCl₃): δ 7.82 (dd, J=15.2 & 10.8 Hz, 1H), 7.32-7.24 (m, 1H), 7.18 (dd, J=15.2 & 8.8 Hz, 1H), 6.93 (t, J=8.8 Hz, 2H), 6.0 (s, 1H), 5.32 (dd, J=9.6 & 4.0 Hz, 1H), 4.79 (dd, J=13.2 & 4 Hz, 1H), 4.53 (dd, J=8 & 4 Hz, 1H), 3.66-3.55 (m, 1H), 2.69 (dd, J=13.2 & 11.2 Hz, 1H), 1.89-1.64 (m, 4H), 1.39-1.32 (m, 2H), 1.06-0.93 (m, 12H).

MS (ESI): m/z 379.1 [M+H]⁺

### Example 255

### (3S,6S)-4-(4-(4-chlorophenyl)thiophene-2-carbonyl)-3,6-diisobutylpiperazin-2-one (272)

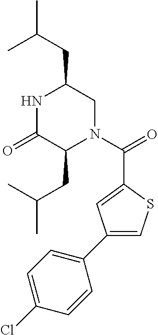

Compound 7 (55 mg, 0.26 mmol) and 4-(4-chlorophenyl)thiophene-2-carboxylic acid (65 mg, 0.28 mmol) were coupled according to the procedure described for compound 70 to give the product 272 (98 mg, 83% yield) as a white solid.

¹H NMR (400 MHz, CDCl₃): δ 7.56-7.54 (m, 2H), 7.50-7.47 (m, 2H), 7.38 (m, 2H), 6.04 (s, 1H), 5.26-5.23 (m, 1H), 4.50-4.30 (m, 1H), 3.66 (s, 1H), 3.20-2.98 (m, 1H), 1.90-1.67 (m, 5H), 1.38-1.30 (m, 3H), 1.01-0.91 (m, 12H).

MS (ESI): m/z 434.1 [M+H]⁺.

### Example 256

### (3S,6S)-3,6-diisobutyl-4-(5-(4-nitrophenyl)isoxazole-3-carbonyl)piperazin-2-one (273)

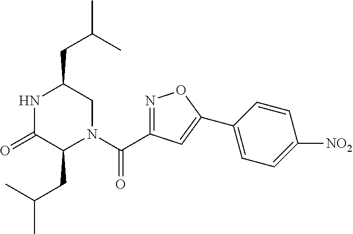

Compound 7 (60 mg, 0.28 mmol) and 5-(4-nitrophenyl)isoxazole-3-carboxylic acid (60 mg, 0.28 mmol) were coupled according to the procedure described for compound 70 to give the product 273 (60.6 mg, 31.6% yield) as an yellow solid.

¹H NMR (400 MHz, CDCl₃): δ 8.37-8.34 (m, 2H), 7.99-7.96 (m, 2H), 7.12 (s, 1H), 6.20 (s, 1H), 5.29-5.25 (m, 1H), 4.88-4.83 (m, 1H), 3.79-3.69 (m, 1H), 3.14 (dd, J=14 & 9.8 Hz, 1H), 1.92-1.69 (m, 4H), 1.42-1.35 (m, 2H), 1.08-0.92 (m, 12H).

MS (ESI): m/z 429.1 [M+H]⁺

### Example 257

### (3S,6S)-4-(5-(2-chlorophenyl)isoxazole-3-carbonyl)-3,6-diisobutylpiperazin-2-one (274)

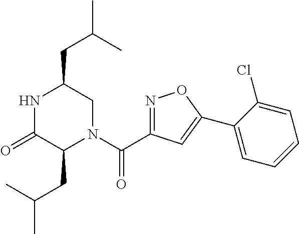

Compound 7 (60 mg, 0.28 mmol) and 5-(2-chlorophenyl)isoxazole-3-carboxylic acid 42 (60 mg, 0.28 mmol) were coupled according to the procedure described for compound 70 to give the product 274 (85 mg, 72% yield) as a white solid.

¹H NMR (400 MHz, CDCl₃): δ 7.96-7.92 (m, 1H), 7.54-7.51 (m, 1H), 7.43-7.39 (m, 2H), 7.24 (s, 1H), 6.19 (d, J=8.8 Hz, 1H), 6.59 (s, 1H), 5.30-5.25 (m, 1H), 4.85-4.78 (m, 1H), 3.79-3.70 (m, 1H), 3.12 (dd, J=10.8 & 14 Hz, 1H), 1.92-1.66 (m, 4H), 1.42-1.34 (m, 2H), 1.09-0.91 (m, 12H).

MS (ESI): m/z 418.2 [M+H]⁺.

### Example 258

### (3S,6S)-4-(5-(2,4-dichlorophenyl)isoxazole-3-carbonyl)-3,6-diisobutylpiperazin-2-one (275)

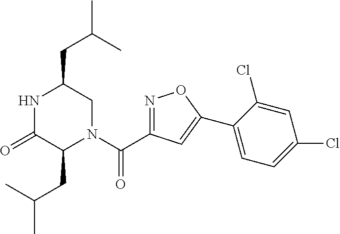

Compound 7 (60 mg, 0.28 mmol) and 5-(2,4-difluorophenyl)isoxazole-3-carboxylic acid (70 mg, 0.28 mmol) were coupled according to the procedure described for compound 70 to furnish 275 (80 mg, 59.5% yield) as a white solid.

¹H NMR (400 MHz, CDCl₃): δ 7.91-7.87 (m, 1H), 7.56-7.54 (m, 1H), 7.41-7.39 (m, 1H), 7.25 (s, 1H), 6.21 (brs, 1H), 5.29-5.22 (m, 1H), 4.84-4.77 (m, 1H), 3.79-3.69 (m, 1H), 3.12 (dd, J=10.8 & 14 Hz, 1H), 1.92-1.66 (m, 4H), 1.42-1.34 (m, 2H) 1.09-0.91 (m, 12H).

MS (ESI): m/z 454.0 (M+H)⁺

### Example 259

### (3S,6S)-4-(5-(3,4-Dichlorophenyl)isoxazole-3-carbonyl)-3,6-diisobutylpiperazin-2-one (276)

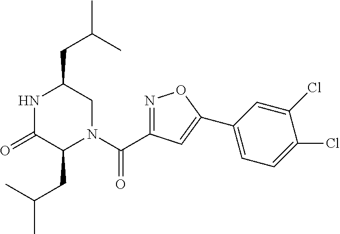

Compound 7 (60 mg, 0.28 mmol) and 5-(2,4-difluorophenyl)isoxazole-3-carboxylic acid (70 mg, 0.28 mmol) were coupled according to the procedure described for compound 70 to furnish 276 (105 mg, 82% yield) as a white solid.

¹H NMR (400 MHz, CD₃OD): δ 7.89-7.88 (m, 1H), 7.63-7.55 (m, 2H), 6.95 (s, 1H), 6.89 (s, 1H), 6.32 (brs, 1H), 5.27-5.24 (m, 1H), 4.86-4.76 (m, 1H), 3.78-3.67 (m, 1H), 3.10 (dd, J=10.8 & 14 Hz, 1H), 1.92-1.66 (m, 4H), 1.42-1.34 (m, 2H), 1.08-0.91 (m, 12H).

MS (ESI): m/z 453.9 [M+H]⁺.

### Example 260

### (3S,6S)-3,6-diisobutyl-4-(5-(4-(methylthio)phenyl)isoxazole-3-carbonyl)piperazin-2-one (277)

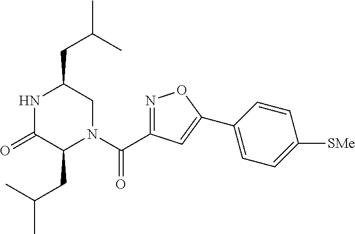

Compound 7 (60 mg, 0.28 mmol) and 5-(4-thiomethylphenyl)isoxazole-3-carboxylic acid (70 mg, 0.28 mmol) were coupled according to the procedure described for compound 70 to furnish 277 (90 mg, 74% yield) as a white solid.

¹H NMR (400 MHz, CDCl₃): δ 7.70-7.67 (m, 2H), 7.31 (d, J=8.4 Hz, 1H), 6.86 (s, 1H), 6.16 (brs, 1H), 5.30-5.26 (m, 1H), 4.85 (dd, J=14 & 4 Hz, 1H), 3.78-3.67 (m, 1H), 3.10 (dd, J=10.8 & 14 Hz, 1H), 2.52 (s, 3H), 1.91-1.67 (m, 4H), 1.42-1.33 (m, 2H), 1.08-0.90 (m, 12H).

MS (ESI): m/z 431.0 [M+H]⁺.

### Example 261

### (3S,6S)-4-(2-(5-(4-fluorophenyl)isoxazol-3-yl)acetyl)-3,6-diisobutylpiperazin-2-one (278)

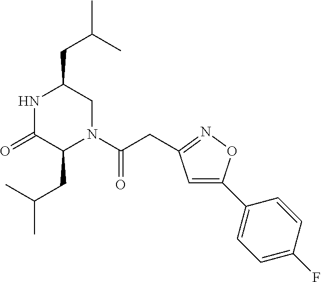

Compound 7 (30 mg, 0.14 mmol) and 2-[5-(4-fluorophenyl)isoxazol-3-yl]acetic acid (30 mg, 0.14 mmol) were coupled according to the procedure described for compound 195 to furnish 278 (20 mg, 34% yield) as a white solid.

¹H NMR (400 MHz, CD₃OD): δ 7.68-7.66 (d, J=8.4 Hz, 2H), 7.41 (dd, J=2.4 & 8.8 Hz, 2H), 6.41 (d, J=10.8 Hz, 1H), 6.06 (brs, 1H), 5.19 (dd, J=4 & 9.6 Hz, 0.5H), 4.69 (dd, J=4 & 13.6 Hz, 0.5H), 4.35 (dd, J=5.6 & 8 Hz, 0.5H), 4.35 (dd, J=4 & 14.4 Hz, 0.5H), 3.58-3.46 (m, 1H), 3.13-3.01 (m, 2H), 2.88-2.81 (m, 1H), 3.21 (dd, J=11, 14.2 Hz, 1H), 1.88-1.52 (m, 4H), 1.38-1.28 (m, 2H), 1.00-0.89 (m, 12H).

MS (ESI): m/z 416.0 [M+H]⁺.

### Example 262

### (3S,6S)-4-(2-(5-(4-fluorophenyl)isoxazol-3-yl)acetyl)-3,6-diisobutylpiperazin-2-one (279)

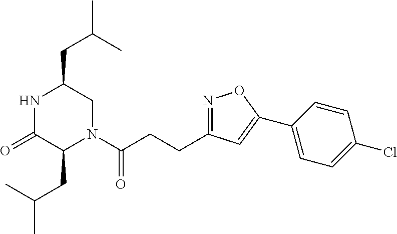

Compound 7 (50 mg, 0.24 mmol) and 3-[5-(4-fluorophenyl)isoxazol-3-yl]-propionic acid (60 mg, 0.24 mmol) were coupled according to the procedure described for compound 195 to furnish 279 (75 mg, 71% yield) as a white solid.

¹H NMR (400 MHz, CD₃OD): δ 7.76-7.72 (m, 2H), 7.18-7.12 (m, 2H), 6.55 (s, 1H), 5.75 (brs, 1H), 5.16 (dd, J=4 & 9.6 Hz, 0.5H), 4.70 (dd, J=4 & 13.6 Hz, 0.5H), 4.45-4.42 (m, 0.5H), 4.04 (dd, J=4 & 14.4 Hz, 0.5H), 3.95-3.72 (m, 2H), 3.35-3.28 (m, 1H), 3.07 (dd, J=7.2 & 14 hz, 1H), 2.88-2.81 (m, 1H), 1.96-1.56 (m, 6H), 1.36-1.24 (m, 2H), 1.05-0.86 (m, 12H).

MS (ESI): m/z 447.0 [M+H]⁺.

### Example 263

### (3S,6S)-3-isobutyl-6-neopentyl-4-((1R,2R)-2-phenylcyclopropanecarbonyl)piperazin-2-one (280)

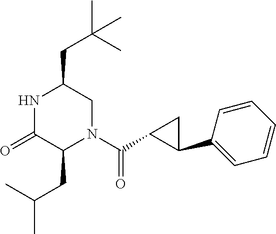

Compound 15 (60 mg, 0.27 mmol) and (1R,2R)-2-phenylcyclopropanecarboxylic acid 54 (50 mg, 0.31 mmol) were coupled according to the procedure described for the preparation of compound 70 to furnish 280 (70 mg, 64% yield) as a white solid.

¹H NMR (400 MHz, CDCl₃): δ 7.31-7.06 (m, 5H), 5.77 (brs, 1H), 5.17 (dd, J=4 & 10 Hz, 0.5H), 4.66 (dd, J=4 & 13.2 Hz, 0.5H), 4.54 (dd, J=4 & 10 Hz, 0.5H), 4.04 (dd, J=4 & 13.2 Hz, 0.5H), 3.63-3.56 (m, 1H), 2.67-2.53 (m, 2H), 1.95-1.58 (m, 6H), 1.48-1.28 (m, 3H), 1.33-1.28 (m, 1H) and 1.02-0.93 (m, 15H).

MS (ESI): m/z 371.1 (M+H)⁺

### Example 264

### Synthesis of (3S,6S)-3,6-dineopentyl-4-((1R,2R)-2-phenylcyclopropanecarbonyl)-piperazin-2-one (281)

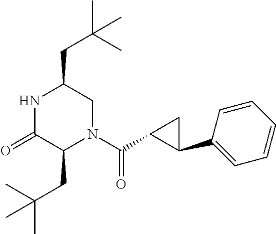

### Step 1: (3S,6S)-3,6-dineopentylpiperazin-2-one (282)

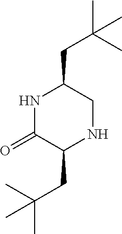

Synthesized from FMOC-gamma-methyl-l-leucine (15.0 g, 0.04 mol) and gamma-methyl-L-Leucine methyl ester (3.5 g, 19.35 mmol) by the method described for the compound 7 in Scheme II to afford the 282 (1.6 g, overall yield: 35.3%) as a white solid.

¹H NMR (DMSO-d6): δ 7.37 (s, 1H), 3.30 (dd, J=4.0, 5.6 Hz, 1H), 3.07 (dd, J=4, 9.6 Hz, 1H), 2.83 (dd, J=4, 12.8 Hz, 1H), 2.56 (dd, J=6, 13.2 Hz, 1H), 1.90 (s, 1H), 1.74-1.79 (m, 1H), 1.33-1.49 (m, 4H), 0.80-0.90 (m, 15H)

MS (ESI): m/z 227.0 (M+H)⁺

### Step 2

3,6-dineopentylpiperazin-2-one 282 (60 mg, 0.25 mmol) and (1R,2R)-2-phenylcyclopropanecarboxylic acid 54 (50 mg, 0.31 mmol) were coupled according to the procedure described for the compound 70 to furnish 281 (50 mg, 52% yield) as a white solid.

¹H NMR (400 MHz, CDCl₃): δ 7.30-7.08 (m, 5H), 5.68 (brs, 1H), 5.29 (dd, J=4 & 10 Hz, 0.5H), 4.68-4.61 (m, 1H), 4.02 (dd, J=4 & 14.4 Hz, 0.5H), 3.66-3.59 (m, 1H), 3.19 (dd, J=11.2 & 14.4 Hz, 0.5H), 2.76 (dd, J=11.2 & 14.4 Hz, 0.5H), 2.63-2.49 (m, 1H), 1.91-1.54 (m, 6H), 1.47-1.28 (m, 3H), 1.04-0.92 (m, 18H).

MS (ESI): m/z 385.1 (M+H)⁺

### Example 265

### (3S,6S)-4-(5-(4-ethylphenyl)isoxazole-3-carbonyl)-3,6-diisobutylpiperazin-2-one (283)

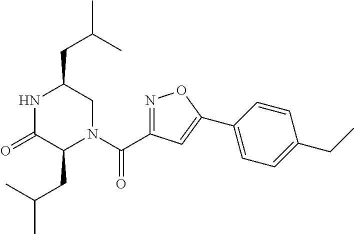

Compound 7 (70 mg, 0.33 mmol) and 5-(4-ethylphenyl)isoxazole-3-carboxylic acid (80 mg, 0.35 mmol) were coupled according to the procedure described for compound 70 to furnish 283 (115 mg, 84.8% yield) as a white solid.

¹H NMR (400 MHz, CDCl₃): δ 7.71-7.69 (m, 2H), 7.33-7.30 (m, 2H), 6.86 (s, 1H), 6.29 (brs, 1H), 5.31-5.25 (m, 1H), 4.89-4.77 (m, 1H), 3.78-3.67 (m, 1H), 3.10 (dd, J=10.8 & 14.0 Hz, 0.5H), 2.82 (dd, J=10.8 & 14.0 Hz, 0.5H), 2.73-2.67 (m, 2H), 1.91-1.67 (m, 4H), 1.41-1.23 (m, 6H), 1.08-0.91 (m, 12H).

MS (ESI): m/z 412.2 [M+H]⁺.

### Example 266

### (3S,6S)-4-(5-(4-cyclopropylphenyl)isoxazole-3-carbonyl)-3,6-diisobutylpiperazin-2-one (284)

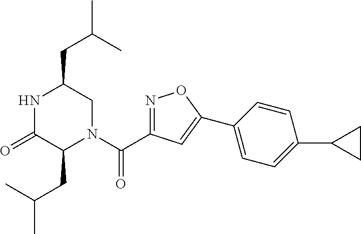

Compound 7 (70 mg, 0.33 mmol) and 5-(4-cyclopropylphenyl)isoxazole-3-carboxylic acid (80 mg, 0.35 mmol) were coupled according to the procedure described for compound 70 to furnish 284 (120 mg, 85.9% yield) as a white solid.

¹H NMR (400 MHz, CDCl₃): δ 7.67-7.64 (m, 2H), 7.16-7.13 (m, 2H), 6.83 (s, 1H), 6.38 (brs, 1H), 5.29-5.25 (m, 1H), 4.88-4.76 (m, 1H), 3.77-3.67 (m, 1H), 3.09 (dd, J=10.8 & 13.6 Hz, 0.5H), 2.82 (dd, J=10.8 & 13.6 Hz, 0.5H), 2.73-2.67 (m, 2H), 1.98-1.67 (m, 5H), 1.42-1.23 (m, 3H), 1.08-0.91 (m, 14H) and 0.77-0.73 (m, 3H).

MS (ESI): m/z 424.2 [M+H]⁺.

### Example 267

### (3S,6S)-3,6-diisobutyl-4-(5-phenyl-1,3,4-oxadiazole-2-carbonyl)piperazin-2-one (285)

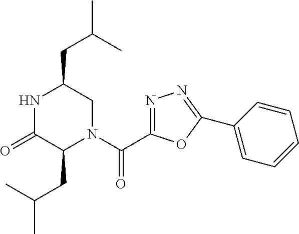

Compound 7 (40 mg, 0.19 mmol) and 2-phenyl-1,2,4-oxadiazole-5-carboxylic acid (38 mg, 0.20 mmol) were coupled according to the procedure described for compound 70 to furnish 285 (15 mg, 20.7% yield) as a white solid.

¹H NMR (400 MHz, CDCl₃): δ 8.07 (dd, J=8 & 3.2 Hz, 1H), 7.56 (t, J=8 Hz, 1H), 7.45-7.41 (m, 3H), 6.10 (d, J=8 Hz, 1H), 5.21 (m, 1H), 4.92 (dd, J=9.2 & 4 Hz, 0.5H), 4.71 (dd, J=9.2 & 4 Hz, 0.5H), 4.44 (dd, J=12.8 & 4 Hz, 0.5H), 4.20 (12.8 & 4 Hz, 0.5H), 3.91-3.75 (m, 1H). 3.15 (dd, 15.2 & 11.2 Hz, 0.5H), 3.00 (dd, J=15.2 & 11.2 Hz, 0.5H), 2.09-1.66 (m, 8H), 1.44-1.36 (m, 3H) and 1.14-0.94 (m, 18H).

MS (ESI): m/z 485.4 [M+H]⁺.

### Example 268

### (3S,6S)-3,6-diisobutyl-4-(7-methoxy-4,5-dihydronaphtho[1,2-b]thiophene-2-carbonyl)piperazin-2-one (286)

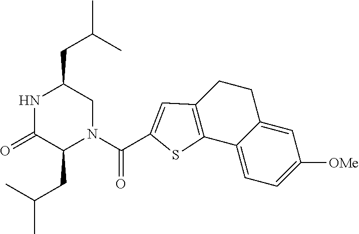

Compound 7 (60 mg, 0.28 mmol) and 7-methoxy-4,5-dihydronaphtho[1,2-b]thiophene-2-carboxylic acid (70 mg, 0.28 mmol) were coupled according to the procedure described for compound 70 to furnish 286 (90 mg, 70% yield) as a white solid.

¹H NMR (400 MHz, CDCl₃): δ 7.29 (d, J=8 Hz, 1H), 7.13 (s, 1H), 6.77-6.74 (m, 2H), 6.40 (brs, 1H), 522-5.10 (m, 1H), 4.56-4.44 (m, 0.5H), 3.80 (s, 3H), 3.70-3.62 (m, 1H), 2.95-2.76 (m, 7H), 1.90-1.64 (m, 4H), 1.38-1.33 (m, 2H) and 0.95-0.80 (m, 12H).

MS (ESI): m/z 455.1 [M+H]⁺.

### Example 269

### (3S,6S)-4-(5-(3,5-difluorophenyl)isoxazole-3-carbonyl)-3,6-diisobutylpiperazin-2-one (287)

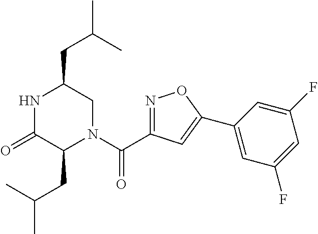

Compound 7 (60 mg, 0.28 mmol) and 5-(3,5-difluorophenyl)isoxazole-3-carboxylic acid 45 (65 mg, 0.29 mmol) were coupled according to the procedure described for compound 70 to furnish 287 (95 mg, 80% yield) as a white solid.

¹H NMR (400 MHz, CDCl₃): δ 7.34-7.25 (m, 2H), 6.97-6.88 (m, 2H), 6.56 (brs, 1H), 5.26-5.23 (m, 1H), 4.85-4.75 (m, 1H), 3.77-3.67 (m, 1H), 3.11 (dd, 14 & 10.8 Hz, 0.5H), 2.83 (dd, J=14 & 10.8 Hz, 0.5H), 1.92-1.68 (m, 4H), 1.50-1.32 (m, 2H), 1.07-0.91 (m, 12H).

MS (ESI): m/z 420.1 (M+H)⁺

### Example 270

### (3S,6S)-6-(cyclopropylmethyl)-4-(5-(2,4-difluorophenyl)isoxazole-3-carbonyl)-3-isobutylpiperazin-2-one (288)

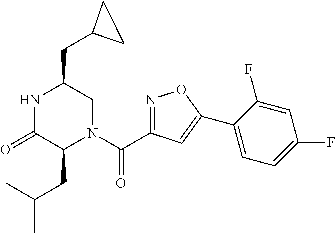

Compound 13 (63 mg, 0.3 mmol) and 5-(2,4-difluorophenyl)isoxazole-3-carboxylic acid (70 mg, 0.31 mmol) were coupled according to the procedure described for compound 70 to furnish 288 (95 mg, 72% yield) as a white solid.

¹H NMR (400 MHz, CDCl₃): δ 7.97-7.91 (m, 1H), 7.06-6.94 (m, 3H), 6.41 (brs, 1H), 5.30-5.21 (s, 1H), 4.88-4.81 (m, 1H), 3.80-3.74 (m, 1H), 3.20 (dd, J=14.4 & 11.2 Hz, 0.5H), 1.92-1.50 (m, 4H), 1.36-1.22 (m, 1H), 1.08-0.75 (m, 6H), 0.72-0.48 (m, 3H) and 0.22-0.99 (m, 2H).

MS (ESI): m/z 418.1 (M+H)⁺

### Example 271

### (3S,6S)-3,6-diisobutyl-4-(7-methoxy-4,5-dihydronaphtho[2,1-d]isoxazole-3-carbonyl)piperazin-2-one (289)

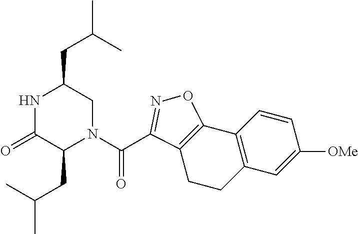

Compound 7 (43 mg, 0.2 mmol) and 7-methoxy-4,5-dihydronaphtho[2,1-d]isoxazole-3-carboxylic acid (50 mg, 0.2 mmol) were coupled according to the procedure described for compound 70 to furnish 289 (60 mg, 64% yield) as a white solid.

¹H NMR (400 MHz, CDCl₃): δ 7.64-7.61 (m, 1H), 6.85-6.82 (m, 2H), 5.85 (brs, 1H), 5.31-5.20 (m, 1H), 4.82-4.72 (m, 1H), 3.84 (s, 3H), 3.75-3.68 (m, 1H), 3.12-2.77 (m, 5H), 1.92-1.58 (m, 4H), 1.42-1.31 (m, 2H), 1.08-0.71 (m, 12H).

MS (ESI): m/z 440.2 [M+H]⁺.

### Example 272

### (3S,6S)-4-(5-(4-fluorophenyl)-1,2,4-oxadiazole-3-carbonyl)-3-isobutyl-6-propylpiperazin-2-one (290)

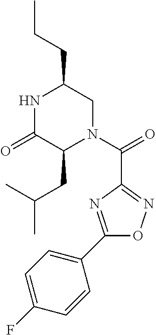

According to the method described for the preparation of compound 89, compound 16 (95.2 mg, 0.48 mmol) was coupled with 5-(4-fluorophenyl)-1,2,4-oxadiazole-3-carboxylic acid 66 (100 mg, 0.48 mmol) to give the product 290 (96.3 mg, 52% yield) as colorless oil.

¹HNMR (CDCl₃, 400 MHz): δ 8.23-8.18 (m, 2H), 7.27-7.22 (m, 2H), 6.50 (s, 1H), 5.30-5.26 (m, 1H) 4.85-4.82 (m, 1H), 3.77-3.65 (m, 1H), 3.19-3.12 (m, 1H), 1.93-1.69 (m, 3H), 1.57-1.31 (m, 4H), 1.09-0.95 (m, 9H).

MS (ESI): m/z 389.1 [M+H]⁺.

### Example 273

### (3S,6S)-6-cyclohexyl-4-(5-(4-fluorophenyl)-1,2,4-oxadiazole-3-carbonyl)-3-isobutylpiperazin-2-one (291)

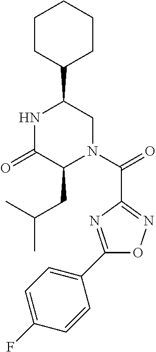

According to the method described for the preparation of compound 89, compound 18 (114.4 mg, 0.48 mmol) was coupled with 5-(4-fluorophenyl)-1,2,4-oxadiazole-3-carboxylic acid 66 (100 mg, 0.48 mmol) to give the product 291 (70.1 mg, 34% yield) as a white solid.

¹HNMR (CDCl₃, 400 MHz): δ 8.23-8.17 (m, 2H), 7.27-7.22 (m, 2H), 6.28 (s, 1H), 5.30-5.26 (m, 1H) 4.84-4.80 (m, 1H), 4.31 (dd, J=4, 14 Hz, 1H), 3.25 (dd, J=11.2, 14 Hz, 1H), 1.92-1.67 (m, 9H), 1.50-1.37 (m, 1H), 1.32-0.97 (m, 10H).

MS (ESI): m/z 429.1 [M+H]⁺.

### Example 274

### (3S,6S)-4-(5-(4-Fluorophenyl)-1,2,4-oxadiazole-3-carbonyl)-3-isobutyl-6-phenylpiperazin-2-one (292)

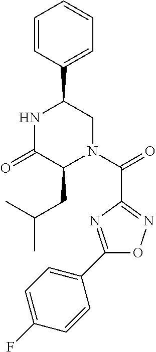

According to the method described for compound 89, 11 (223 mg, 0.96 mmol) was coupled with 5-(4-fluorophenyl)-1,2,4-oxadiazole-3-carboxylic acid 66 (200 mg, 0.96 mmol) to give the product 292 (183.6 mg, 45.3% yield) as a white solid.

¹H NMR (400 MHz, CDCl3): δ 8.24-8.19 (2H, m), 7.43-7.34 (5H, m), 7.27-7.23 (2H, m), 6.47 (1H, s), 5.37-5.33 (1H, m), 4.93-4.89 (1H, m), 4.41-4.36 (1H, m), 3.40-3.34 (1H, m), 1.98-1.74 (3H, m), 1.10 (3H, d, J=6.4 Hz), 0.99 (3H, d, J=6.4 Hz).

MS (ESI): m/z 423.1 [M+H]⁺.

### Example 275

### (3S,6S)-4-(5-(4-Fluorophenyl)-1,2,4-oxadiazole-3-carbonyl)-3,6-diisobutylpiperazin-2-one (293)

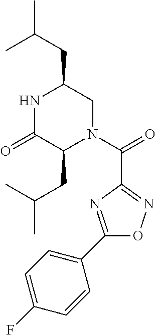

According to the method described for compound 89, 7 (223 mg, 0.96 mmol) was coupled with 5-(4-fluorophenyl)-1,2,4-oxadiazole-3-carboxylic acid 66 (200 mg, 0.96 mmol) to give the product 293 (135 mg, 70% yield) as a white solid.

¹H NMR (400 MHz, CDCl3): δ 8.23-8.18 (2H, m), 7.28-7.22 (2H, m), 6.33 (1H, s), 5.29-5.26 (1H, m), 4.32-4.24 (1H, m), 3.86-3.78 (1H, m), 3.16-3.10 (1H, m), 1.94-1.64 (4H, m), 1.43-1.32 (2H, m), 1.09-0.89 (12H, m).

MS (ESI): m/z 403.1 [M+H]⁺.

### Example 276

### (3S,6S)-6-Cyclopentyl-4-(5-(4-fluorophenyl)-1,2,4-oxadiazole-3-carbonyl)-3-isobutylpiperazin-2-one (294)

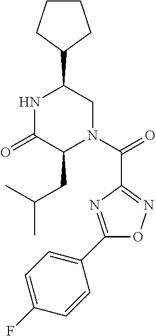

According to the method described for compound 89, 17 (107.7 mg, 0.48 mmol) was coupled with 5-(4-fluorophenyl)-1,2,4-oxadiazole-3-carboxylic acid 66 (100 mg, 0.48 mmol) to give the product 294 (30.5 mg, 15.3% yield) as colorless oil.

¹H NMR (400 MHz, CDCl3): δ 8.24-8.18 (2H, m), 7.27-7.23 (2H, m), 5.95 (1H, s), 5.32-5.24 (1H, m), 4.38-4.28 (1H, m), 3.59-3.42 (1H, m), 3.21-3.16 (1H, m), 1.94-1.48 (12H, m), 1.10 (3H, d, J=6.4 Hz), 0.99 (3H, d, J=6.4 Hz).

MS (ESI): m/z 415.2 [M+H]⁺.

### Example 277

### (3S,6S)-4-(5-(4-Fluorophenyl)-1,2,4-oxadiazole-3-carbonyl)-3-isobutyl-6-isopropylpiperazin-2-one (295)

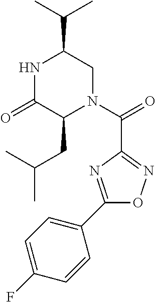

According to the method described for compound 89, 8 (95.2 mg, 0.48 mmol) was coupled with 5-(4-fluorophenyl)-1,2,4-oxadiazole-3-carboxylic acid 66 (100 mg, 0.48 mmol) to give the product 295 (108.6 mg, 58.2% yield) as white solids.

¹H NMR (400 MHz, CDCl3): δ 8.23-8.18 (2H, m), 7.27-7.22 (2H, m), 6.44 (1H, s), 5.30-5.27 (1H, m), 4.84-4.81 (1H, m), 3.60-3.53 (1H, m), 3.25-3.19 (1H, m), 1.92-1.68 (4H, m), 1.09-0.96 (12H, m).

MS (ESI): m/z 389.1 [M+H]⁺.

### Example 278

### (3S,6R)-4-(5-(4-Fluorophenyl)-1,2,4-oxadiazole-3-carbonyl)-3-isobutyl-6-((methylthio)methyl)piperazin-2-one (296)

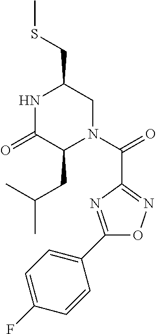

According to the method described for compound 89, 10 (103.8 mg, 0.48 mmol) was coupled with 5-(4-fluorophenyl)-1,2,4-oxadiazole-3-carboxylic acid 66 (100 mg, 0.48 mmol) to give the product 296 (58.2 mg, 30% yield) as colorless oil.

¹H NMR (400 MHz, CDCl3): δ 8.23-8.19 (2H, m), 7.27-7.23 (2H, m), 6.50 (1H, s), 5.33-5.30 (1H, m), 4.91-4.86 (1H, m), 3.90-3.83 (1H, m), 3.23 (1H, dd, J=10.8, 14 Hz), 2.70-2.66 (1H, m), 2.44-2.38 (1H, m), 2.13 (3H, s), 1.94-1.70 (3H, m), 1.09 (3H, d, J=6.4 Hz), 0.99 (3H, d, J=6.4 Hz).

MS (ESI): m/z 407.1 [M+H]⁺.

### Example 279

### (3S,6S)-4-[(1R,2R)-2-(4-Fluoro-phenyl)-cyclopropanecarbonyl]-6-cyclopropyl-methyl-3-isobutyl-piperazin-2-one (297)

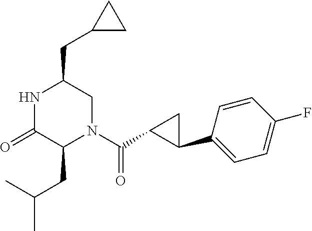

Compound 13 (45 mg, 0.21 mmol) and trans (R,R)[2-(p-fluoro)phenyl]-cyclopropyl-1-carboxylic acid 60 (40 mg, 0.22 mmol) were coupled according to the method described for the preparation of compound 70 to furnish product 297 (70 mg, 83.4% yield) as a white solid.

¹H NMR (400 MHz, CDCl₃): δ 7.10-6.95 (m, 4H), 6.03 (brs, 1H), 5.20 (dd, J=9.6 & 4.0 Hz, 0.5H), 4.75 (dd, J=14.4 & 4.0 Hz, 0.5H), 4.56 (dd, J=9.6 & 4.0 Hz, 0.5H), 4.12 (dd, J=14.4 & 4.0 Hz, 0.5H), 3.65-3.58 (m, 1H), 3.14 (dd, J=14.4 & 11.2 Hz, 0.5H), 2.67 (dd, J=14.4 & 11.2 Hz, 0.5H), 2.55-2.50 (m, 1H), 1.89-1.53 (m, 6H), 1.28-1.21 (m, 2H), 1.02-0.93 (m, 6H), 0.72-0.46 (m, 3H) and 0.20-0.07 (m, 2H).

MS (ESI): m/z 373.1 [M+H]⁺

### Example 280

### (3S,6S)-4-[(1R,2R)-2-(2,4-fluoro-phenyl)-cyclopropanecarbonyl]-3,6-diisobutyl-piperazin-2-one (298)

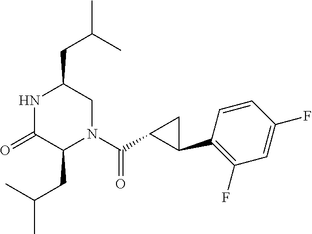

Compound 7 (42 mg, 0.20 mmol) and trans (R,R)[2-(2-fluoro-4-chloro)phenyl]-cyclopropyl-1-carboxylic acid 64 (40 mg, 0.20 mmol) were coupled according to the method described for the preparation of compound 70 to furnish product 298 (55 mg, 70.8% yield) as a white solid.

¹H NMR (400 MHz, CDCl₃): δ 7.06-6.75 (m, 3H), 6.20 (brs, 1H), 5.20 (dd, J=9.6 & 4 Hz, 0.5H), 4.70 (dd, J=13.6 & 4.0 Hz, 0.5H), 4.57 (dd, J=9.6 & 4.0 Hz, 0.5H), 4.05 (dd, J=13.6 & 4.0 Hz, 0.5H), 3.61-3.54 (m, 1H), 3.12 (dd, J=14.0 & 11.2 Hz, 0.5H), 2.65-2.50 (m, 1.5H), 1.99-1.63 (m, 6H), 1.35-1.28 (m, 3H) and 1.02-0.92 (m, 12H).

MS (ESI): m/z 393.2 [M+H]⁺

### Example 281

### (3S,6R)-4-((1R,2R)-2-(4-fluorophenyl)cyclopropanecarbonyl)-3-isobutyl-6-((methylthio)methyl)piperazin-2-one(299)

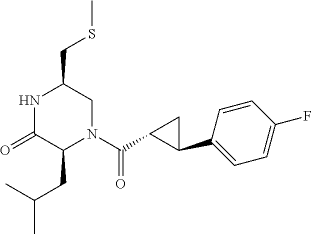

Compound 10 substrate (43 mg, 0.20 mmol) and trans (R,R)[2-(p-fluoro)phenyl]-cyclopropyl-1-carboxylic acid 60 (37 mg, 0.21 mmol) were coupled according to the method described for the preparation of compound 70 to furnish product 299 (40 mg, 50.5% yield) as a white solid.

¹H NMR (400 MHz, CDCl₃): δ 7.10-6.95 (m, 4H), 6.43 (brs, 1H), 5.22 (dd, J=8.8 & 4.0 Hz, 0.5H), 4.73 (dd, J=13.2 & 4.0 Hz, 0.5H), 4.56 (dd, J=8.8 & 4.0 Hz, 0.5H), 4.16 (dd, J=13.2 & 4.0 Hz, 0.5H), 3.69-3.56 (m, 1H), 3.14 (dd, J=14.0 & 11.2 Hz, 0.5H), 2.76-2.66 (m, 1.5H), 2.57-2.49 (m, 1H), 2.42-2.29 (m, 1H), 2.08 (s, 3H), 1.91-1.56 (m, 5H), 1.30-1.25 (m, 1H) and 1.02-0.94 (m, 6H).

MS (ESI): m/z 379.1 [M+H]⁺

### Example 282

### (3S,6R)-4-((1R,2R)-2-(4-Chlorophenyl)cyclopropanecarbonyl)-3-isobutyl-6-((methylthio)methyl)piperazin-2-one(300)

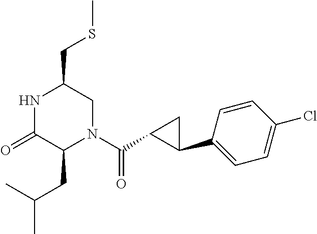

Compound 10 (43 mg, 0.20 mmol) and trans (R,R)[2-(p-chloro)phenyl]-cyclopropyl-1-carboxylic acid 59 (37 mg, 0.21 mmol) were coupled according to the method described for the preparation of compound 70 to furnish product 300 (65 mg, 82.8% yield) as a white solid.

¹H NMR (400 MHz, CDCl₃): δ 7.27-7.23 (m, 2H), 7.07-6.98 (m, 2H), 6.49 (brs, 1H), 5.21 (dd, J=8.8 & 4.0 Hz, 0.5H), 4.74 (dd, J=13.6 & 4.0 Hz, 0.5h), 4.55 (dd, J=8.8 & 4.0 Hz, 0.5H), 4.15 (dd, J=13.6 & 4.0 Hz, 0.5H), 3.71-3.56 (m, 1H), 3.16 (dd, J=14.4 & 9.6 Hz, 0.5H), 2.75-2.66 (m, 1.5H), 2.53-2.30 (m, 2H), 2.08 (s, 3H), 1.91-1.64 (m, 5H), 1.32-1.24 (m, 1H) and 1.02-0.93 (m, 6H).

MS (ESI): m/z 395.7 [M+H]⁺

### Example 283

### (3S,6S)-4-((1R,2R)-2-(4-chlorophenyl)cyclopropanecarbonyl)-3-isobutyl-6-phenyl-piperazin-2-one (301)

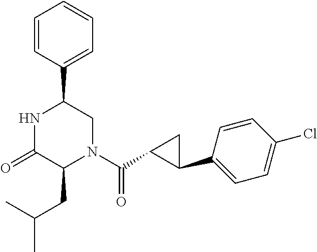

Compound 11 (46 mg, 0.20 mmol) and trans (R,R)[2-(p-chloro)phenyl]-cyclopropyl-1-carboxylic acid 59 (40 mg, 0.20 mmol) were coupled according to the method described for the preparation of compound 70 to furnish product 301 (64 mg, 78.7% yield) as a white solid.

¹H NMR (400 MHz, CDCl₃): δ 7.42-7.22 (m, 7H), 7.04-7.01 (m, 2H), 5.97 (brs, 1H), 5.30 (dd, J=10.0 & 4.0 Hz, 0.5H), 4.77 (dd, J=13.6 & 4.4 Hz, 0.5H), 4.69-4.61 (m, 1.5H), 4.13 (dd, J=13.6 & 4.0 Hz, 0.5H), 3.36 (dd, J=14.4 & 9.6H, 0.5 Hz), 2.86 (dd, J=14.4 & 9.6 Hz, 0.5H), 2.59-2.52 (m, 1H), 1.99-1.64 (m, 5H), 1.38-1.24 (m, 1H) and 1.05-0.96 (m, 6H).

MS (ESI): m/z 411.1 [M+H]⁺

### Example 284

### Synthesis of (3S,6S)-3,6-Diisobutyl-4-(7-fluoro-4,5-dihydronaphtho[2,1-d]isoxazole-3-carbonyl)piperazin-2-one (302)

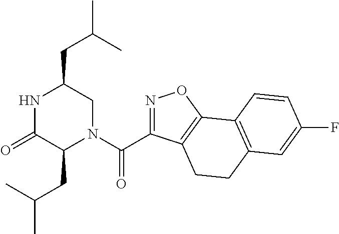

### Step 1: 7-Fluoro-4,5-dihydronaphtho[2,1-d]isoxazole-3-carboxylic acid (303)

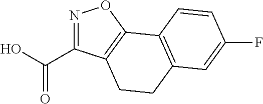

Synthesized from 6-Fluoro-tetralone (0.5 g, 3.05 mmol) according to the method described in the literature for 4,5-dihydronaphtho[2,1-d]isoxazole-3-carboxylic acid (Antiviral Chemistry & Chemotherapy, 16, 41-61, 2005) to give 303 (130 mg, 18% overall yield from 6-fluoro-tetralone).

¹H NMR (400 MHz, CDCl₃): δ 7.75-7.71 (dd, J=8.8 & 5.2 Hz, 1H), 7.24-7.14 (m, 2H), 3.13 (t, J=8.0 Hz, 2H) and 3.01 (t, J=8.0 Hz, 2H).

MS (ESI): m/z 234.0 [M+H]⁺

### Step 2

Compound 7 (22 mg, 0.1 mmol) and 7-fluoro-4,5-dihydronaphtho[2,1-d]isoxazole-3-carboxylic acid 303 (23 mg, 0.1 mmol) were coupled according to the procedure described for compound 70 to furnish 302 (28 mg, 63.2% yield) as a white solid.

¹H NMR (400 MHz, CDCl₃): δ 7.69-7.65 (m, 1H), 7.04-6.99 (m, 2H), 6.04 (brs, 1H), 5.31-5.19 (m, 1H), 4.80-4.72 (m, 1H), 3.77-3.68 (m, 1H), 3.13-2.78 (m, 5H), 1.92-1.64 (m, 4H), 1.42-1.31 (m, 2H) and 1.08-0.72 (m, 12H).

MS (ESI): m/z 428.1 [M+H]⁺.

### Example 285

### (3S,6S)-4-(7-fluoro-4,5-dihydronaphtho[2,1-d]isoxazole-3-carbonyl)-3-isobutyl-6-propylpiperazin-2-one (304)

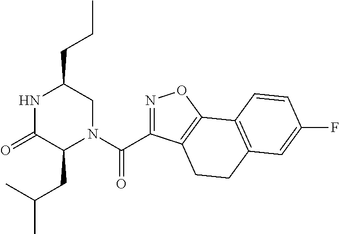

Compound 16 (20 mg, 0.1 mmol) and 7-fluoro-4,5-dihydronaphtho[2,1-d]isoxazole-3-carboxylic acid 303 (23 mg, 0.1 mmol) were coupled according to the procedure described for compound 70 to furnish 304 (25 mg, 60% yield) as a white solid.

¹H NMR (400 MHz, CDCl₃): δ 7.68-7.64 (m, 1H), 7.03-6.99 (m, 2H), 6.17 (brs, 1H), 5.30-5.17 (m, 1H), 4.82-4.72 (m, 1H), 3.69-3.63 (m, 1H), 3.15-2.80 (m, 5H), 1.92-1.68 (m, 3H), 1.56-1.36 (m, 4H) and 1.08-0.72 (m, 9H).

MS (ESI): m/z 414.1 [M+H]⁺.

### Example 286

### (3S,6S)-6-cyclopentyl-4-(7-fluoro-4,5-dihydronaphtho[2,1-d]isoxazole-3-carbonyl)-3-isobutylpiperazin-2-one (305)

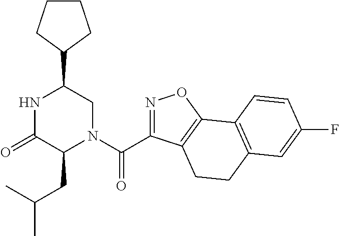

Compound 17 (23 mg, 0.1 mmol) and 7-fluoro-4,5-dihydronaphtho[2,1-d]isoxazole-3-carboxylic acid 303 (23 mg, 0.1 mmol) were coupled according to the procedure described for compound 70 to furnish 305 (30 mg, 66.6% yield) as a white solid.

¹H NMR (400 MHz, CDCl₃): δ 7.68-7.64 (m, 1H), 7.03-6.99 (m, 2H), 6.04 (brs, 1H), 5.29-5.16 (m, 1H), 4.83-4.75 (m, 1H), 3.52-3.38 (m, 1H), 3.17-2.80 (m, 4H), 1.92-1.54 (m, 11H), 1.36-1.24 (m, 2H) and 1.08-0.73 (m, 6H).

MS (ESI): m/z 440.2 [M+H]⁺.

### Example 287

### (3S,6S)-4-(7-fluoro-4,5-dihydronaphtho[2,1-d]isoxazole-3-carbonyl)-3-isobutyl-6-phenylpiperazin-2-one (306)

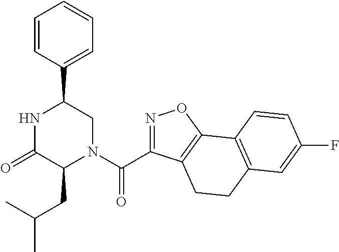

Compound 11 (23 mg, 0.1 mmol) and 7-fluoro-4,5-dihydronaphtho[2,1-d]isoxazole-3-carboxylic acid 303 (23 mg, 0.1 mmol) were coupled according to the procedure described for compound 70 to furnish 306 (25 mg, 56.4% yield) as a white solid.

¹H NMR (400 MHz, CDCl₃): δ 7.69-7.65 (m, 1H), 7.43-7.36 (m, 5H), 7.04-6.95 (m, 2H), 6.09 (brs, 1H), 5.39-5.32 (m, 1H), 4.91-4.76 (m, 2H), 3.33 (dd, J=13.6 & 10.4 Hz, 1H), 3.09-2.85 (m, 4H), 2.02-1.68 (m, 3H), 1.36-1.24 (m, 2H) and 1.11-0.77 (m, 6H).

MS (ESI): m/z 448.1 [M+H]⁺.

### Example 288

### (3S,6S)-3,6-diisobutyl-4-(4H-thieno[3,2-c]chromene-2-carbonyl)piperazin-2-one (307)

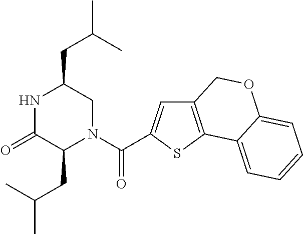

Compound 7 (42 mg, 0.20 mmol) and 4H-thieno[3,2-c]chromene-2-carboxylic acid (45 mg, 0.20 mmol) were coupled according to the procedure described for compound 70 to furnish 307 (45 mg, 50.7% yield) as a white solid.

¹H NMR (400 MHz, CDCl₃): δ 7.30 (dd, J=7.6 & 1.6 Hz, 1H), 7.22-7.17 (m, 1H), 7.07 (s, 1H), 6.99-6.93 (m, 2H), 6.12 (brs, 1H), 528-5.06 (m, 3H), 4.45 (brs, 1H), 3.65 (brs, 1H), 3.05 (brs, 1H), 1.90-1.66 (m, 4H), 1.38-1.32 (m, 2H) and 0.97-0.90 (m, 12H).

MS (ESI): m/z 427.1 [M+H]⁺.

### Example 289

### (3S,6S)-4-(8-fluoro-4H-thieno[3,2-c]chromene-2-carbonyl)-3,6-diisobutylpiperazin-2-one (308)

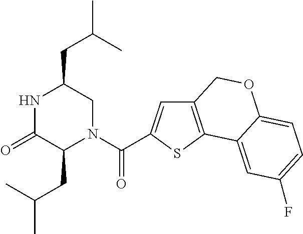

Compound 7 (42 mg, 0.20 mmol) and 4H-thieno[3,2-c]chromene-2-carboxylic acid (50 mg, 0.20 mmol) were coupled according to the procedure described for compound 70 to furnish 308 (55 mg, 62.5% yield) as a white solid.

¹H NMR (400 MHz, CDCl₃): δ 7.07 (s, 1H), 7.01-6.98 (m, 1H), 6.89-6.86 (m, 2H), 6.20 (brs, 1H), 524-5.02 (m, 3H), 4.41 (brs, 1H), 3.63 (brs, 1H), 3.07 (brs, 1H), 1.90-1.66 (m, 4H), 1.40-1.32 (m, 2H) and 0.97-0.90 (m, 12H).

MS (ESI): m/z 445.1 [M+H]⁺.

### Example 290

### (3S,6S)-4-[(1R,2R)-2-(3,4-Difluoro-phenyl)-cyclopropanecarbonyl]-3-isobutyl-6-isopropyl-piperazin-2-one (309)

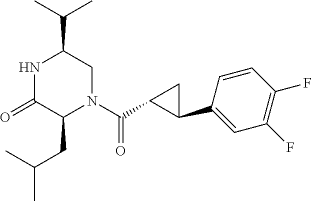

Compound 8 (20 mg, 0.10 mmol) and trans (R,R)[2-(3,4-di-fluoro)phenyl]-cyclopropyl-1-carboxylic acid 61 (20 mg, 0.10 mmol) were coupled according to the method described for the preparation of compound 70 to furnish product 309 (36 mg, 84.9% yield) as a white solid.

¹H NMR (400 MHz, CDCl₃): δ 7.10-7.02 (m, 1H), 6.92-6.76 (m, 2H), 6.10 (brs, 1H), 5.17 (dd, J=9.6 & 4.0 Hz, 0.5H), 4.68 (dd, J=13.6 & 4.0 Hz, 0.5H), 4.53 (dd, J=9.6 & 4 Hz, 0.5H), 4.03 (dd, J=13.6 & 4.0 Hz, 0.5H), 3.37-3.28 (m, 1H), 3.18 (dd, J=14.0 & 11.2 Hz, 0.5H), 2.67 (0.5H, dd, J=14.0 & 11.2 Hz, 0.5H), 2.54-2.47 (m, 1H), 1.90-1.58 (m, 5H), 1.26-1.22 (m, 1H), 1.27-1.23 (m, 1H) and 1.04-0.92 (m, 12H).

MS (ESI): m/z 379.1 [M+H]⁺

### Example 291

### (3S,6S)-4-((1R,2R)-2-(4-(tert-butyl)phenyl)cyclopropanecarbonyl)-6-cyclopentyl-3-isobutylpiperazin-2-one (310)

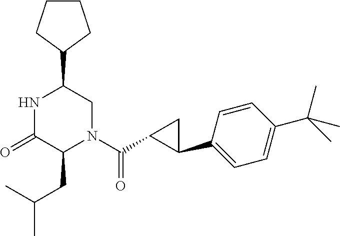

Compound 17 (45 mg, 0.20 mmol) and trans (R,R)[2-(p-tert-butyl)phenyl]-cyclopropyl-1-carboxylic acid 65 (44 mg, 0.20 mmol) were coupled according to the method described for the preparation of compound 70 to furnish product 310 (65 mg, 70.2% yield) as a white solid.

¹H NMR (400 MHz, CDCl₃): δ 7.33 (d, J=8.4 Hz, 1H), 7.29 (d, J=8.4 Hz, 1H), 7.07 (d, J=8.4 Hz, 1H), 6.99 (d, J=8.4 Hz, 1H), 5.90 (s, 1H), 5.21 (dd J=9.6 & 4 Hz, 0.5H), 4.73 (dd, J=13.6 & 4 Hz, 0.5H), 4.57 (dd, J=9.6 & 4 Hz, 0.5H), 4.07 (dd, J=13.6 & 4 Hz, 0.5H), 3.35-3.25 (m, 1H), 3.09 (dd, J=14.0 & 11.2 Hz, 0.5H), 2.65 (dd, J=13.6 & 11.2 Hz, 0.5H), 2.53-2.48 (m, 1H), 1.92-1.58 (m, 12H), 1.30-1.23 (m, 12H) and 1.02-0.92 (m, 6H).

MS (ESI): m/z 425.2 [M+H]⁺

### Example 292

### (3S,6S)-4-((1R,2R)-2-(4-(tert-butyl)phenyl)cyclopropanecarbonyl)-3-isobutyl-6-isopropylpiperazin-2-one (311)

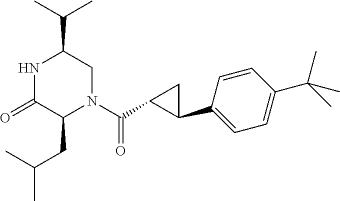

Compound 8 (40 mg, 0.20 mmol) and trans (R,R)[2-(p-tert-butyl)phenyl]-cyclopropyl-1-carboxylic acid 65 (44 mg, 0.20 mmol) were coupled according to the method described for the preparation of compound 70 to furnish product 311 (60 mg, 65.3% yield) as a white solid.

¹H NMR (400 MHz, CDCl₃): δ 7.33-7.25 (m, 2H), 7.07-6.99 (m, 2H), 5.95 (brs, 1H), 5.19 (dd, J=9.6 & 4.0 Hz, 0.5H), 4.71 (dd, J=13.6 & 4.0 Hz, 0.5H), 4.56 (dd, J=9.6 & 4 Hz, 0.5H), 4.06 (dd, J=13.6 & 4.0 Hz, 0.5H), 3.38-3.27 (m, 1H), 3.13 (dd, J=14.0 & 11.2 Hz, 0.5H), 2.69 (dd, J=14.0 & 11.2 Hz, 0.5H), 2.53-2.47 (m, 1H), 1.92-1.58 (m, 6H), 1.30 (m, 10H) and 1.02-0.92 (m, 12H).

MS (ESI): m/z 415.2 [M+H]⁺

### Example 293

### (3S,6S)-4-((1R,2R)-2-(4-(tert-butyl)phenyl)cyclopropanecarbonyl)-3-isobutyl-6-phenylpiperazin-2-one (312)

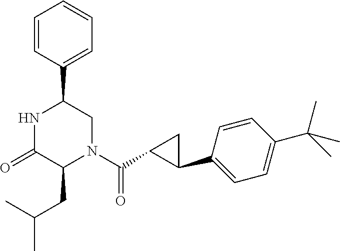

Compound 11 substrate (46 mg, 0.20 mmol) and trans (R,R)[2-(p-tert-butyl)phenyl]-cyclopropyl-1-carboxylic acid 65 (44 mg, 0.20 mmol) were coupled according to the method described for the preparation of compound 70 to furnish product 312 (64 mg, 78.7% yield) as a white solid.

¹H NMR (400 MHz, CDCl₃): δ 7.43-7.31 (m, 7H), 7.06-7.02 (m, 2H), 5.92 (brs, 1H), 5.32 (dd, J=10.0 & 4.0 Hz, 0.5H), 4.79 (dd, J=14.0 & 4.4 Hz, 0.5H), 4.70-4.62 (m, 1.5H), 4.17 (dd, J=14.0 & 4.4 Hz, 0.5H), 3.35 (dd, J=14.4 & 11.2 Hz, 0.5 Hz), 2.86 (dd, J=14.4 & 11.2 Hz, 0.5H), 2.58-2.53 (m, 1H), 2.00-1.66 (m, 5H), 1.38-1.24 (m, 10H) and 1.05-0.94 (m, 6H).

MS (ESI): m/z 433.1 [M+H]⁺

### Example 294

### (3S,6S)-4-(3-(4-chlorophenyl)isoxazole-5-carbonyl)-3-isobutyl-6-propylpiperazin-2-one (313)

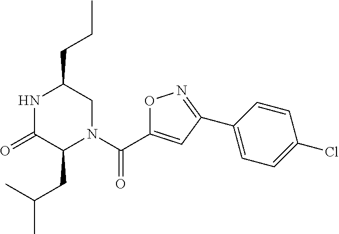

Compound 16 (50 mg, 0.25 mmol) and 3-(4-chlorophenyl)isoxazole-5-carboxylic acid (57 mg, 0.25 mmol) were coupled according to the procedure described for compound 70 to furnish compound 313 (83 mg, 81% yield) as colorless solid.

¹H NMR (400 MHz, CDCl₃): δ 7.76-7.74 (d, J=8.4 Hz, 2H), 7.48-7.45 (m, 2H), 7.17 (s, 1H), 6.10 (s, 1H), 5.24-4.90 (m, 1H), 4.74-4.42 (m, 1H), 3.70-3.64 (m, 1H), 3.23-2.86 (m, 1H), 1.93-1.85 (m, 1H), 1.80-1.69 (m, 2H), 1.56-1.37 (m, 4H), 1.07-0.83 (m, 9H).

MS (ESI): m/z 403.9 [M+H]⁺

### Example 295

### (3S,6S)-4-(3-(4-chlorophenyl)isoxazole-5-carbonyl)-6-cyclopentyl-3-isobutyl-piperazin-2-one (314)

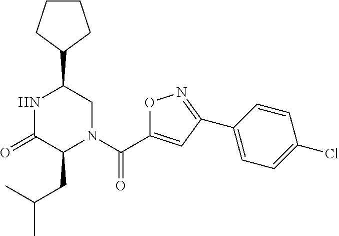

Compound 17 (50 mg, 0.22 mmol) and 3-(4-chlorophenyl)isoxazole-5-carboxylic acid (50 mg, 0.22 mmol) were coupled according to the procedure described for compound 70 to furnish compound 314 (81 mg, 85% yield) as colorless solid.

¹H NMR (400 MHz, CDCl₃): δ 7.76-7.74 (d, J=8.4 Hz, 2H), 7.48-7.45 (m, 2H), 7.18-7.08 (m, 1H), 5.95 (d, J=7.6 Hz, 1H), 5.24-4.89 (m, 1H), 4.77-4.45 (m, 1H), 3.53-3.44 (m, 1H), 3.25-2.89 (m, 1H), 1.93-1.69 (m, 10H), 1.29-1.27 (m, 2H), 1.08-0.83 (m, 6H).

MS (ESI): m/z 430.0 [M+H]⁺

### Example 296

### (3S,6S)-4-(3-(4-chlorophenyl)isoxazole-5-carbonyl)-6-cyclohexyl-3-isobutylpiperazin-2-one (315)

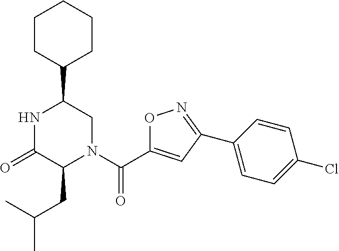

Compound 18 (50 mg, 0.21 mmol) and 3-(4-chlorophenyl)isoxazole-5-carboxylic acid (47 mg, 0.21 mmol) were coupled according to the procedure described for compound 70 to furnish compound 315 (47 mg, 51% yield) as colorless solid.

¹H NMR (400 MHz, CDCl₃): δ 7.77-7.74 (d, J=8.4 Hz, 2H), 7.48-7.44 (m, 2H), 7.17-7.08 (m, 1H), 6.02 (d, J=8.4 Hz, 1H), 5.24-4.88 (m, 1H), 4.73-4.43 (m, 1H), 3.54-3.44 (m, 1H), 3.32-2.95 (m, 1H), 1.91-1.71 (m, 8H), 1.44-1.39 (m, 1H), 1.29-1.12 (m, 3H), 1.07-0.83 (m, 6H).

MS (ESI): m/z 444.0 [M+H]⁺

### Example 297

### (3S,6S)-4-(3-(4-chlorophenyl)isoxazole-5-carbonyl)-3-isobutyl-6-phenylpiperazin-2-one (316)

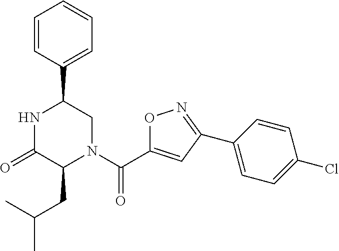

Compound 11 (50 mg, 0.22 mmol) and 3-(4-chlorophenyl)isoxazole-5-carboxylic acid (48 mg, 0.22 mmol) were coupled according to the procedure described for compound 70 to furnish compound 316 (71 mg, 75% yield) as colorless solid.

¹H NMR (400 MHz, CDCl₃): δ 7.77-7.75 (d, J=8.4 Hz, 2H), 7.48-7.37 (m, 7H), 7.21-7.15 (m, 1H), 6.05 (d, J=10 Hz, 1H), 5.33-5.01 (m, 1H), 4.88-4.84 (m, 1H), 4.80-4.51 (m, 1H), 3.45-3.08 (m, 1H), 2.04-1.95 (m, 1H), 1.90-1.83 (m, 1H), 1.80-1.72 (m, 1H), 1.10.0.88 (m, 6H).

MS (ESI): m/z 437.9 [M+H]⁺

### Example 298

### Synthesis of (S)-4-(5-(4-fluorophenyl)isoxazole-3-carbonyl)-3,6-diisobutyl-3,4-dihydropyrazin-2(1H)-one (317)

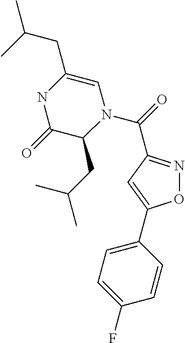

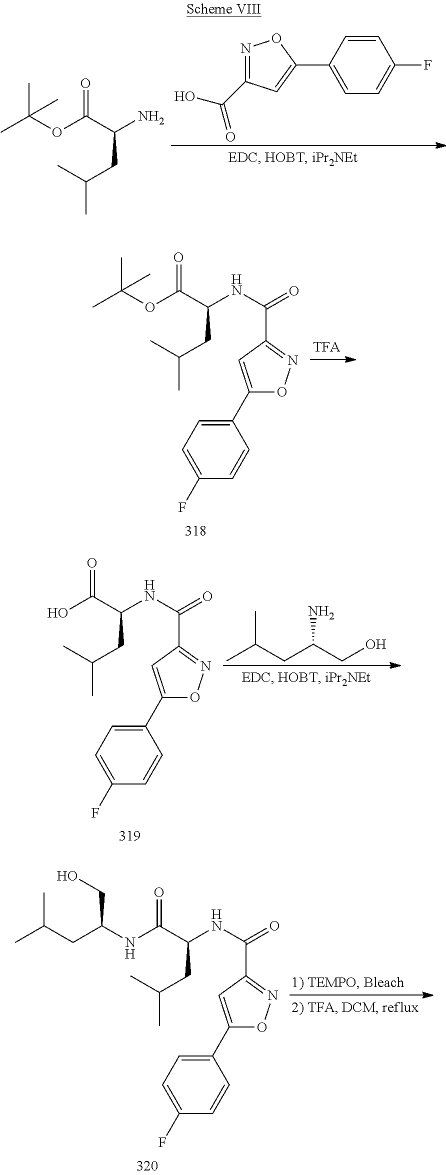

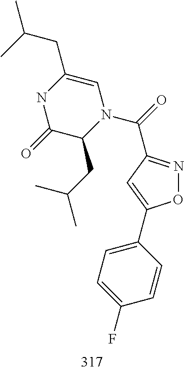

### Step 1: (S)-tert-Butyl 2-(5-(4-fluorophenyl)isoxazole-3-carboxamido)-4-methyl-pentanoate (318)

A mixture of (S)-tert-butyl 2-amino-4-methylpentanoate (749.12 mg, 4 mmol), 5-(4-fluorophenyl)isoxazole-3-carboxylic acid (994 mg, 4.8 mmol), EDC (920.16 mg, 4.8 mmol), HOBT (675.65 mg, 5 mmol), iPr₂NEt (1.39 mL, 8 mmol) in CH₃CN (40 mL) was stirred at room temperature overnight. After removal of solvent, the residue was purified by column to give (S)-tert-Butyl 2-(5-(4-fluorophenyl)isoxazole-3-carboxamido)-4-methylpentanoate (318) as white solid (1.2 g, 80% yield).

¹H NMR (400 MHz, CDCl3): δ 7.80-7.77 (2H, m), 7.20-7.16 (3H, m), 6.90 (1H, s), 4.72-4.67 (1H, m), 1.78-1.63 (3H, m), 1.49 (9H, s), 0.99-0.97 (6H, m).

MS (ESI): m/z 377.1 [M+H]⁺.

### Step 2: (S)-2-(5-(4-Fluorophenyl)isoxazole-3-carboxamido)-4-methylpentanoic acid (319)

To a solution of 318 (658.1 mg, 1.75 mmol) in dichloromethane (24 mL) was added TFA (8 mL). The mixture was stirred at room temperature for 5 h. The reaction mixture was concentrated in vacuo to give (S)-2-(5-(4-Fluorophenyl)isoxazole-3-carboxamido)-4-methylpentanoic acid (319) as brownish solid. (560 mg, 100% yield).

¹H NMR (400 MHz, CDCl3): δ 11.1 (1H, s), 7.78 (2H, dd, J=5, 8.6 Hz), 7.55 (1H, d, J=8 Hz), 7.18 (2H, t, J=8.6 Hz), 6.99 (1H, s), 4.87-4.82 (1H, m), 1.86-1.76 (3H, m), 0.99 (6H, d, J=6 Hz).

MS (ESI): m/z 321.0 [M+H]⁺.

### Step 3: 5-(4-Fluorophenyl)-N—((S)-1-(((S)-1-hydroxy-4-methylpentan-2-yl)amino)-4-methyl-1-oxopentan-2-yl)isoxazole-3-carboxamide (320)

A mixture of (S)-(+)leucinol (0.152 mL, 1.178 mmol), 319 (452.5 mg, 1.414 mmol), EDC (271.1 mg, 1.414 mmol), HOBT (199 mg, 1.4725 mmol), iPr₂NEt (0.82 mL, 4.712 mmol) in CH₃CN (20 mL) was stirred at room temperature overnight. The reaction mixture was concentrated in vacuo, and the residue was purified by column to give 5-(4-Fluorophenyl)-N—((S)-1-(((S)-1-hydroxy-4-methylpentan-2-yl)amino)-4-methyl-1-oxopentan-2-yl)isoxazole-3-carboxamide (320) as colorless oil (415 mg, 70% yield).

¹H NMR (400 MHz, CDCl3): δ 7.76-7.70 (2H, m), 7.64 (1H, d, J=8.4 Hz), 7.15-7.11 (2H, m), 6.91-6.87 (2H, m), 4.74-4.68 (1H, m), 4.12-4.00 (1H, m), 3.69-3.49 (3H, m), 1.80-1.65 (3H, m), 1.62-1.29 (3H, m), 0.94-0.83 (12H, m).

MS (ESI): m/z 420.2 [M+H]⁺.

### Step 4: (S)-4-(5-(4-Fluorophenyl)isoxazole-3-carbonyl)-3,6-diisobutyl-3,4-dihydro-pyrazin-2(1H)-one (317)

To the ice-cooled solution of 320 (204.65 mg, 0.488 mmol) in dichloromethane (2 mL) was added TEMPO (1 mg, 0.005 mmol) and NaHCO₃ (aq., sat., 1.02 mL). Sodium hypochlorite solution (6%, 1.3 mL) was then added dropwise to the solution. After 4 h stirring at room temperature for 4 h, the reaction mixture was diluted with dichloromethane (20 mL) and washed with sat. aqueous NH₄Cl. The organic layer was dried over anhydrous Na₂SO₄. After filtration and concentration in vacuo, the crude aldehyde was used directly for next step.

To a solution of crude aldehyde (183 mg, 0.44 mmol) in dichloromethane (12 mL) was added TFA (0.1 mL, 1.32 mmol). The reaction mixture was heated to reflux for 2 days. After concentration in vacuo, the residue was purified by column to give (S)-4-(5-(4-Fluorophenyl)isoxazole-3-carbonyl)-3,6-diisobutyl-3,4-dihydropyrazin-2(1H)-one (317) as pale yellow oil (88 mg, 50% yield).

¹H NMR (400 MHz, CDCl3): δ 7.81-7.78 (2H, m), 7.38 (1H, s), 7.21-7.17 (2H, m), 6.88 (1H, s), 6.67 (1H, s), 5.33 (1H, t, J=7.2 Hz), 2.16-1.97 (2H, m), 1.74-1.56 (4H, m), 1.04-0.95 (12H, m).

MS (ESI): m/z 400.1 [M+H]⁺.

### Example 299

### (3S,6S)-4-(5-(4-fluorophenyl)isoxazole-3-carbonyl)-3-isobutyl-1-methyl-6-phenylpiperazin-2-one (321)

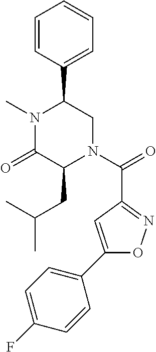

To a solution of 178 (50 mg, 0.12 mmol) in DMF (2.5 mL) was added sodium hydride (60% dispersion in mineral oil, 7.2 mg, 0.18 mmol) and iodomethane (0.1 mL, 1.6 mmol) at rt. The reaction mixture was stirred at rt for 1 h. After removal of solvents in vacuo, the residue was purified by column to give the product 321 as pale yellow oil (54 mg, 100% yield).

¹H NMR (400 MHz, CDCl3): δ 7.82-7.77 (2H, m), 7.44-7.37 (3H, m), 7.30-7.27 (2H, m), 7.21-7.16 (2H, m), 6.88 (1H, s), 5.45-5.38 (1H, m), 4.96-4.91 (1H, m), 4.76 (1H, dd, J=5.2, 10.8 Hz), 3.47 (1H, dd, J=11.2, 14.6 Hz), 2.72 (3H, s), 2.06-1.70 (3H, m), 1.12 (3H, d, J=6.4 Hz), 1.00 (3H, d, J=6.4 Hz).

MS (ESI): m/z 436.1 [M+H]⁺.

### Example 300

### (3S,6S)-1-ethyl-4-(5-(4-fluorophenyl)isoxazole-3-carbonyl)-3-isobutyl-6-phenylpiperazin-2-one(322)

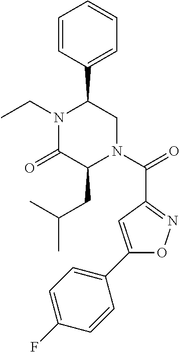

According to the method described for compound 321, 178 (50 mg, 0.12 mmol) was treated with iodoethane (0.1 mL, 1.24 mmol) to give the product 322 as a colorless oil (26.2 mg, 48% yield)

¹H NMR (400 MHz, CDCl3): δ 7.82-7.77 (2H, m), 7.44-7.37 (3H, m), 7.32-7.30 (2H, m), 7.21-7.16 (2H, m), 6.88 (1H, s), 5.39-5.35 (1H, m), 4.95-4.89 (1H, m), 4.85-4.81 (1H, m), 3.94-3.84 (1H, m), 3.24 (1H, dd, J=11.2, 14.6 Hz), 2.74-2.64 (1H, m), 2.06-1.71 (3H, m), 1.12 (3H, d, J=6.4 Hz), 1.02-0.96 (6H, m).

MS (ESI): m/z 450.1 [M+H]⁺.

### Example 301

### (3S,6S)-4-(5-(4-fluorophenyl)isoxazole-3-carbonyl)-3-isobutyl-6-phenyl-1-propylpiperazin-2-one(323)

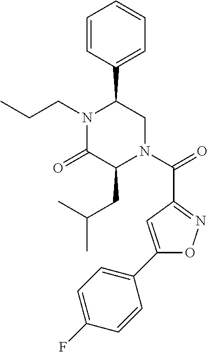

According to the method described for compound 321, 178 (50 mg, 0.12 mmol) was treated with iodopropane (0.1 mL, 1.02 mmol) to give the product 323 as a colorless oil (23.8 mg, 43% yield).

¹H NMR (400 MHz, CDCl3): δ 7.80-7.77 (2H, m), 7.44-7.37 (3H, m), 7.31-7.29 (2H, m), 7.21-7.17 (2H, m), 6.88 (1H, s), 5.40-5.36 (1H, m), 4.94-4.89 (1H, m), 4.84-4.80 (1H, m), 3.89-3.81 (1H, m), 3.48 (1H, dd, J=11.2, 14.4 Hz), 2.58-2.48 (1H, m), 2.06-1.70 (3H, m), 1.55-1.36 (2H, m), 1.12 (3H, d, J=6.8 Hz), 1.01 (3H, d, J=6.8 Hz), 0.75 (3H, t, J=7.4 Hz).

MS (ESI): m/z 464.3 [M+H]⁺.

### Example 302

### (3S,6S)-4-[(1R,2R)-2-(2,4-difluoro-phenyl)-cyclopropanecarbonyl]-6-ethyl-3-isobutyl-piperazin-2-one (324)

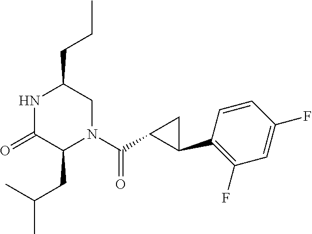

Compound 16 (40 mg, 0.20 mmol) and trans (R,R)[2-(2,4-difluorophenyl]-cyclopropyl-1-carboxylic acid 64 (45 mg, 0.23 mmol) were coupled according to the method described for the preparation of compound 71 to furnish product 324 (38 mg, 49.8% yield) as a white solid.

¹H NMR (400 MHz, CDCl₃): δ 7.05-6.75 (m, 3H), 5.98 (s, 1H), 5.23 (dd, J=10.0 & 4.0 Hz, 0.5H), 4.73 (dd, J=13.6 & 4.0 Hz, 0.5H), 4.53 (dd, J=10.0 & 4.0 Hz, 0.5H), 4.04 (dd, J=13.6 & 4.0 Hz, 0.5H), 3.56-3.47 (m, 1H), 3.11 (dd, J=14.4 & 11.2 Hz, 0.5H), 2.68-2.62 (m, 1H), 2.46 (dd, J=14.4 & 11.2 Hz, 0.5H), 1.96-1.61 (m, 5H), 1.48-1.24 (m, 5H) and 1.02-0.68 (m, 9H).

MS (ESI): m/z 379.0 [M+H]⁺

### Example 303

### (3S,6S)-6-cyclopentyl-4-((1R,2R)-2-(2,4-difluorophenyl)cyclopropanecarbonyl)-3-isobutylpiperazin-2-one (325)

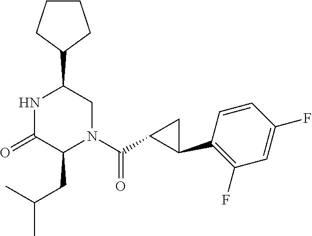

Compound 17 (45 mg, 0.20 mmol) and trans (R,R)[2-(2,4-difluorophenyl]-cyclopropyl-1-carboxylic acid 64 (45 mg, 0.23 mmol) were coupled according to the method described for the preparation of compound 71 to furnish product 325 (25 mg, 30.8% yield) as a white solid.

¹H NMR (400 MHz, CDCl₃): δ 7.03-6.75 (m, 3H), 6.02 (brs, 1H), 5.22 (dd J=10.4 & 4.0 Hz, 0.5H), 4.75 (dd, J=13.6 & 4.0 Hz, 0.5H), 4.53 (dd, J=10.4 & 4.0 Hz, 0.5H), 4.03 (dd, J=13.6 & 4.0 Hz, 0.5H), 3.32-3.26 (m, 1H), 3.15 (dd, J=14.0 & 11.2 Hz, 0.5H), 2.70-2.62 (m, 1H), 1.92-1.58 (m, 12H), 2.45 (dd, J=13.6 & 11.2 Hz, 0.5H), 1.94-1.54 (m, 12H), 1.42-1.16 (m, 3H) and 1.02-0.67 (4 d, J=6 Hz, 6H).

MS (ESI): m/z 405.2 [M+H]⁺

### Example 304

### (3S,6S)-6-cyclohexyl-4-((1R,2R)-2-(2,4-difluorophenyl)cyclopropanecarbonyl)-3-isobutylpiperazin-2-one (326)

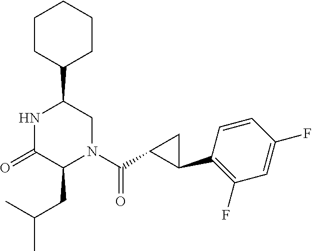

Compound 7 (50 mg, 0.21 mmol) and trans (R,R)[2-(2,4-difluorophenyl]-cyclopropyl-1-carboxylic acid 64 (45 mg, 0.23 mmol) were coupled according to the method described for the preparation of compound 70 to furnish product 326 (19 mg, 20.6% yield) as a white solid.

¹H NMR (400 MHz, CDCl₃): δ 7.03-6.74 (m, 3H), 6.02 (brs, 1H), 5.23 (dd, J=10.4 & 4.0 Hz, 0.5H), 4.71 (dd, J=13.6 & 4.4 Hz, 0.5H), 4.52 (dd, J=10.4 & 4.0 Hz, 0.5H), 4.03 (dd, J=13.6 & 4.4 Hz, 0.5H), 3.38-3.27 (m, 1H), 3.19 (dd, J=13.6 & 11.2 Hz, 0.5H), 2.74 (dd, J=13.6 & 11.2 Hz, 0.5H), 2.66-2.62 (m, 0.5H), 2.50-2.40 (m, 0.5H), 1.96-1.88 (m, 1H), 1.82-1.56 (m, 10H), 1.39-0.67 (m, 12H).

MS (ESI): m/z 419.1 [M+H]⁺

### Example 305

### (3S,6S)-4-[(1R,2R)-2-(2,4-difluoro-phenyl)-cyclopropanecarbonyl]-3-isobutyl-6-phenyl-piperazin-2-one (327)

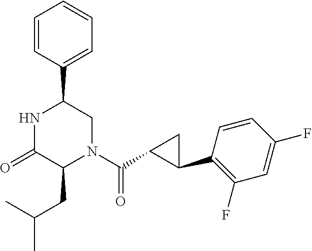

Compound 11 substrate (47 mg, 0.20 mmol) and trans (R,R)[2-(2-fluoro-4-chloro)phenyl]-cyclopropyl-1-carboxylic acid 64 (45 mg, 0.23 mmol) were coupled according to the method described for the preparation of compound 70 to furnish product 327 (35 mg, 39.8% yield) as a white solid.

¹H NMR (400 MHz, CDCl₃): δ 7.43-7.33 (m, 4H), 7.22 (dd, J=7.2 & 2.4 Hz, 1H), 7.10-6.76 (m, 3H), 6.06 (d, J=14 Hz, 1H), 5.35 (dd, J=10.0 & 4.0 Hz, 0.5H), 4.82 (dd, J=14.4 & 4.0 Hz, 0.5H), 4.69-4.62 (m, 1.5H), 4.12 (dd, J=14.4 & 4.0 Hz, 1H), 3.34 (dd, J=14.4 & 11.2 Hz, 0.5H), 2.87 (dd, J=14.4 & 11.2 Hz, 0.5H), 2.70-2.65 (m, 0.5H), 2.54-2.49 (m, 0.5H), 1.98-1.58 (m, 6H), 1.46-1.28 (m, 1H) and 1.04-0.71 (m, 5H).

MS (ESI): m/z 413.1 [M+H]⁺

### Example 306

### (3S,6S)-4-((1R,2R)-2-(4-(tert-butyl)phenyl)cyclopropanecarbonyl)-6-cyclohexyl-3-isobutylpiperazin-2-one (328)

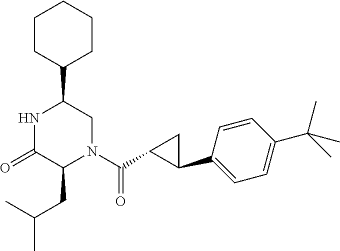

Compound 18 (48 mg, 0.20 mmol) and trans (R,R)[2-(p-tert-butyl)phenyl]-cyclopropyl-1-carboxylic acid 65 (44 mg, 0.20 mmol) were coupled according to the method described for the preparation of compound 70 to furnish product 328 (20 mg, 22.6% yield) as a white solid.

¹H NMR (400 MHz, CDCl₃): δ 7.34-7.28 (m, 2H), 7.06 (d, J=8.4 Hz, 1H), 7.01 (d, J=8.4 Hz, 1H), 5.99 (d, J=9.8 Hz, 1H), 5.20 (dd J=9.6 & 4.0 Hz, 0.5H), 4.72 (dd, J=13.6 & 4.0 Hz, 0.5H), 4.56 (dd, J=9.6 & 4.0 Hz, 0.5H), 4.06 (dd, J=13.6 & 4.0 Hz, 0.5H), 3.36-3.27 (m, 1H), 3.17 (dd, J=14.0 & 11.2 Hz, 0.5H), 2.72 (dd, J=13.6 & 11.2 Hz, 0.5H), 2.52-2.47 (m, 1H), 1.93-1.58 (m, 10H), 1.40-0.92 (m, 22H).

MS (ESI): m/z 440.1 [M+H]⁺

### Example 307

### (3S,6S)-4-((1R,2R)-2-(4-bromophenyl)cyclopropanecarbonyl)-3,6-diisobutylpiperazin-2-one (329)

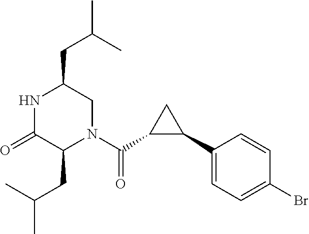

### Step 1: (1R,2R)-2-(4-Bromo-phenyl)-cyclopropanecarboxylic acid (330)

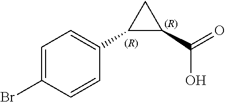

Synthesized from (E)-3-(4-bromophenyl)acrylic acid (20.4 g, 90.30 mmol) by the method described for 55 (Scheme VI) to furnish product 330 (750.11 mg, overall yield: 3.2%) as a white solid.

¹H-NMR (400 MHz, DMSO-d₆) δ 12.31-12.35 (m, 1H), 7.42-7.44 (d, J=8.0 Hz, 1H), 7.11-7.13 (d, J=8.0 Hz, 1H), 2.34-2.39 (m, 1H), 1.77-1.81 (m, 1H), 1.31-1.42 (m, 1H).

MS (ESI): m/z 242.98 [M+H]⁺

### Step 2

Compound 7 (50 mg, 0.24 mmol) and (1R,2R)-2-(4-bromophenyl)-cyclopropanecarboxylic acid 330 (57 mg, 0.24 mmol) were coupled according to the procedure described for compound 70 to furnish compound 329 (79 mg, 77% yield) as a white solid.

¹H NMR (400 MHz, CDCl₃): δ 7.42-7.38 (m, 2H), 7.01-6.93 (m, 2H), 6.00-5.97 (m, 1H), 5.20-4.66 (m, 1H), 4.53-4.00 (m, 1H), 3.63-3.52 (m, 1H), 3.11-2.58 (m, 1H), 2.53-2.47 (m, 1H), 1.91-1.59 (m, 6H), 1.38-1.24 (m, 3H), 1.01-0.91 (m, 6H).

MS (ESI): m/z 435.0 [M+H]⁺

### Example 308

### (3S,6S)-4-[(1R,2R)-2-(4-Bromo-phenyl)-cyclopropanecarbonyl]-3-isobutyl-6-propyl-piperazin-2-one (331)

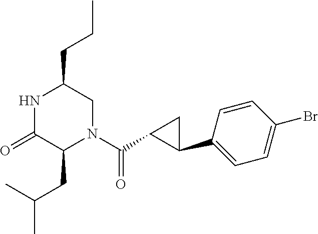

Compound 16 (40 mg, 0.20 mmol) and trans (R,R)[2-(p-bromo)phenyl]-cyclopropyl-1-carboxylic acid 330 (50 mg, 0.21 mmol) were coupled according to the method described for the preparation of compound 70 to furnish product 331 (40 mg, 47.1% yield) as a white solid.

¹H NMR (400 MHz, CDCl₃): δ 7.41-7.36 (m, 2H), 7.04-6.91 (m, 2H), 6.69 (brs, 1H), 5.16 (dd J=9.6 & 4.0 Hz, 0.5H), 4.70 (dd, J=13.6 & 4.0 Hz, 0.5H), 4.51 (dd, J=9.6 & 4.0 Hz, 0.5H), 4.03 (dd, J=14.4 & 4.0 Hz, 0.5H), 3.55-3.44 (m, 1H), 3.08 (dd, J=13.6 & 11.2 Hz, 0.5H), 2.61 (dd, J=13.6 & 11.2 Hz, 0.5H), 2.52-2.46 (m, 1H), 1.92-1.58 (m, 5H), 1.48-1.22 (m, 5H) and 1.08-0.92 (m, 9H).

MS (ESI): m/z 423.0 [M+H]⁺

### Example 309

### (3S,6S)-6-cyclopentyl-4-((1R,2R)-2-(4-bromophenyl)cyclopropanecarbonyl)-3-isobutylpiperazin-2-one (332)

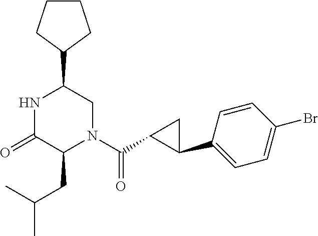

Compound 17 (45 mg, 0.20 mmol) and trans (R,R)[2-(p-bromo)phenyl]-cyclopropyl-1-carboxylic acid 330 (50 mg, 0.21 mmol) were coupled according to the method described for the preparation of compound 70 to furnish product 332 (30 mg, 33.4% yield) as a white solid.

¹H NMR (400 MHz, CDCl₃): δ 7.42-7.37 (m, 2H), 7.04-6.91 (m, 2H), 6.03 (brs, 1H), 5.18 (dd, J=9.6 & 4.0 Hz, 0.5H), 4.72 (dd, J=13.6 & 4.0 Hz, 0.5H), 4.52 (dd, J=9.6 & 4.0 Hz, 0.5H), 4.03 (dd, J=13.6 & 4.0 Hz, 0.5H), 3.35-3.26 (m, 1H), 3.12 (dd, J=14.0 & 11.2 Hz, 0.5H), 2.66 (dd, J=14.0 & 11.2 Hz, 0.5H), 2.52-2.46 (m, 1H), 1.92-1.54 (m, 12H), 1.32-1.16 (m, 3H) and 1.02-0.92 (m, 6H).

MS (ESI): m/z 449.9 [M+2H]⁺

### Example 310

### (3S,6S)-4-[(1R,2R)-2-(4-bromo-phenyl)-cyclopropanecarbonyl]-3-isobutyl-6-phenyl-piperazin-2-one (333)

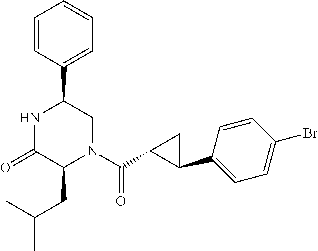

Compound 11 (47 mg, 0.20 mmol) and trans (R,R)[2-(p-bromo)-(phenyl]-cyclopropyl-1-carboxylic acid 330 (45 mg, 0.21 mmol) were coupled according to the method described for the preparation of compound 70 to furnish product 333 (55 mg, 56.7% yield) as a white solid.

¹H NMR (400 MHz, CDCl₃): δ 7.43-7.32 (m, 7H), 6.99-6.96 (m, 2H), 6.04 (brs, 1H), 5.30 (dd, J=10.0 & 4.0 Hz, 0.5H), 4.79 (dd, J=13.6 & 4.0 Hz, 0.5H), 4.69-4.60 (m, 1.5H), 4.12 (dd, J=13.6 & 4.0 Hz, 0.5H), 3.36 (dd, J=14.4 & 11.2 Hz, 0.5H), 2.86 (dd, J=14.4 & 11.2 Hz, 0.5H), 2.56-2.51 (m, 1H), 1.98-1.64 (m, 5H), 1.36-1.27 (m, 1H) and 1.04-0.95 (m, 6H).

MS (ESI): m/z 456.9 [M+H]⁺

### Example 311

### (3S,6S)-4-((1R,2R)-2-(4-(tert-butyl)phenyl)cyclopropanecarbonyl)-3-isobutyl-6-propyl-pylpiperazin-2-one (334)

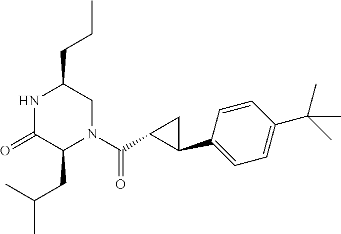

Compound 16 (60 mg, 0.30 mmol) and trans (R,R)[2-(p-tert-butyl)phenyl]-cyclopropyl-1-carboxylic acid 65 (66 mg, 0.30 mmol) were coupled according to the method described for the preparation of compound 70 to furnish product 334 (95 mg, 78.8% yield) as a white solid.

¹H NMR (400 MHz, CDCl₃): δ 7.32 (d, J=8.4 Hz, 1H), 7.29 (m, J=8.4 Hz, 1H), 7.07 (d, J=8.4 Hz, 1H), 7.01 (d, J=8.4 Hz, 1H), 6.23 (d, J=30.8 Hz, 1H), 5.20 (dd, J=9.6 & 4.0 Hz, 0.5H), 4.72 (dd, J=13.6 & 4.0 Hz, 0.5H), 4.56 (dd, J=9.6 & 4 Hz, 0.5H), 4.08 (dd, J=13.6 & 4.0 Hz, 0.5H), 3.55-3.46 (m, 1H), 3.08 (dd, J=14.4 & 11.2 Hz, 0.5H), 2.62 (dd, J=14.4 & 11.2 Hz, 0.5H), 2.53-2.48 (m, 1H), 1.94-1.58 (m, 5H), 1.50-1.28 (m, 14H) and 1.02-0.92 (m, 9H).

MS (ESI): m/z 399.1 [M+H]⁺

### Example 312

### (3S,6S)-6-cyclohexyl-4-((1R,2R)-2-(2,4-difluorophenyl)cyclopropanecarbonyl)-3-isobutylpiperazin-2-one (335)

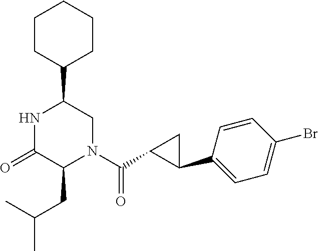

Compound 18 (50 mg, 0.21 mmol) and trans (R,R)[2-(p-bromo)-(phenyl]-cyclopropyl-1-carboxylic acid 330 (50 mg, 0.21 mmol) were coupled according to the method described for the preparation of compound 70 to furnish product 335 (25 mg, 25.8% yield) as a white solid.

¹H NMR (400 MHz, CDCl₃): δ 7.42-6.37 (m, 2H), 7.05-6.92 (m, 2H), 5.94 (brs, 1H), 5.18 (dd, J=9.6 & 4.0 Hz, 0.5H), 4.69 (dd, J=13.6 & 4.4 Hz, 0.5H), 4.52 (dd, J=9.6 & 4.0 Hz, 0.5H), 4.04 (dd, J=13.6 & 4.4 Hz, 0.5H), 3.35-3.27 (m, 1H), 3.20 (dd, J=14.0 & 11.2 Hz, 0.5H), 2.73 (dd, J=14.0 & 11.2 Hz, 0.5H), 2.50-2.46 (m, 1H), 1.92-1.58 (m, 11H), 1.42-1.04 (m, 6H), 1.01-0.92 (m, 6H).

MS (ESI): m/z 463.0 [M+H]⁺

### Example 313

### (3S,6S)-3-(cyclopropylmethyl)-4-(5-(4-fluorophenyl)isoxazole-3-carbonyl)-6-phenylpiperazin-2-one (336)

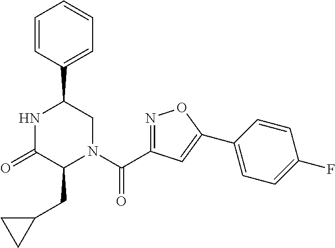

### Step 1: Synthesis of (3S,6S)-3-Cyclopropylmethyl-6-phenyl-piperazin-2-one (337)

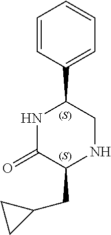

Synthesized from Amino-phenyl-acetic acid (6.70 g, 4.43 mmol) and 2-Amino-3-cyclopropyl-propionic acid methyl ester (5 g, 35 mmol) by the method described for the compound 7 (Scheme II) to afford the product (3S,6S)-3-(cyclopropylmethyl)-6-phenylpiperazin-2-one 337 (1.1 g, overall yield: 10.89%).

¹H NMR (400 MHz, DMSO-d6): δ 7.93 (s, 1H), 7.26-7.36 (m, 5H), 4.48 (dd, J=3.2, 7.2 Hz, 1H), 3.28 (dd, J=3.6, 8.0 Hz, 1H), 3.12 (dd, J=4.4, 8.8 Hz, 1H), 2.84 (dd, J=4.0, 12.8 Hz, 1H), 2.38 (s, 1H), 1.70-1.77 (m, 1H), 1.44-1.51 (m, 1H), 0.85-0.87 (m, 1H), 0.37-0.45 (m, 2H), 0.01-0.19 (m, 2H)

MS (ESI): m/z 231.3 [M+H]⁺

### Step 2

Compound 337 (50 mg, 0.22 mmol) and 5-(4-fluorophenyl)isoxazole-3-carboxylic acid (45 mg, 0.22 mmol) were coupled according to the procedure described for compound 71 to furnish compound 336 (73 mg, 80% yield) as a colorless solid.

¹H NMR (400 MHz, CDCl₃): δ 7.82-7.78 (m, 2H), 7.45-7.35 (m, 5H), 7.22-7.16 (m, 2H), 6.87-6.83 (m, 1H), 6.13-6.11 (m, 1H), 5.57-5.35 (m, 1H), 5.00-4.93 (m, 1H), 4.89-4.76 (m, 1H), 3.54-3.14 (m, 1H), 2.15-1.93 (m, 2H), 1.94-0.70 (m, 1H), 0.53-0.32 (m, 2H), 0.23-0.07 (m, 2H).

MS (ESI): m/z 420.0 [M+H]⁺

### Example 314

### (3S,6S)-3-(cyclopropylmethyl)-6-phenyl-4-((1R,2R)-2-phenylcyclopropanecarbonyl)-piperazin-2-one (338)

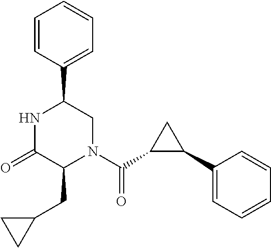

Compound 337 (50 mg, 0.22 mmol) and (1R,2R)-2-phenylcyclopropanecarboxylic acid 54 (35 mg, 0.22 mmol) were coupled according to the procedure described for compound 70 to furnish compound 338 (71 mg, 87% yield) as colorless solid.

¹H NMR (400 MHz, CDCl₃): δ 7.43-7.09 (m, 10H), 6.02 (s, 1H), 5.62-4.84 (m, 1H), 4.76-4.73 (m, 1H), 4.67-4.20 (m, 1H), 3.58-2.89 (m, 1H), 2.56-2.51 (m, 1H), 2.21-2.13 (m, 1H), 2.04-1.71 (m, 3H), 1.40-1.25 (m, 1H), 0.88-0.83 (m, 1H) 0.65-0.47 (m, 2H), 0.30-0.13 (m, 2H).

MS (ESI): m/z 375.0 [M+H]⁺

### Example 315

### (3S,6S)-3-((R)-sec-butyl)-4-(5-(4-fluorophenyl)isoxazole-3-carbonyl)-6-phenylpiperazin-2-one (339)

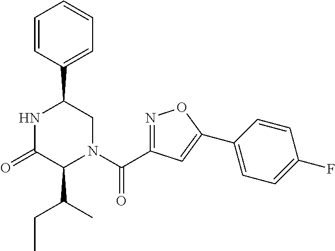

### Step 1: Synthesis of (3S,6S)-3-((R)-sec-butyl)-6-phenylpiperazin-2-one (340)

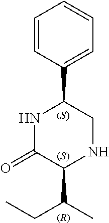

Synthesized from (S)-2-amino-2-phenylacetic acid (30.0 g, 0.19 mol) and (2S,3R)-methyl 2-amino-3-methylpentanoate hydrochloride (4.3 g, 0.029 mol) by the method described for the compound 7 (Scheme II) to afford the product 340 (1.39 g, overall yield: 3.2%).

¹H NMR (400 MHz, DMSO-d₆) δ 7.92 (d, J=2.8 Hz, 1H), 7.24-7.30 (m, 4H), 7.20-7.23 (m, 1H), 4.43 (d, J=3.6, 1H), 3.07-3.12 (m, 2H), 2.87 (dd, J=2.8, 12.8 Hz, 1H), 1.96-2.01 (m, 1H), 1.43-1.49 (m, 1H), 1.14-1.23 (m, 1H), 0.90 (d, J=7.2, 3H), 8.83 (t, J=8.0 Hz, 3H).

MS (ESI): m/z 233.1 [M+H]⁺

### Step 2

Compound 340 (50 mg, 0.22 mmol) and 5-(4-fluorophenyl)isoxazole-3-carboxylic acid (45 mg, 0.22 mmol) were coupled according to the procedure described for compound 71 to furnish compound 339 (68 mg, 75% yield) as colorless solid.

¹H NMR (400 MHz, CDCl₃): δ 7.82-7.77 (m, 2H), 7.46-7.36 (m, 5H), 7.22-7.16 (m, 2H), 6.87-6.82 (m, 1H), 6.06-5.99 (m, 1H), 5.25-5.18 (m, 1H), 4.96-4.92 (m, 1H), 4.88-4.76 (m, 1H), 3.44-3.05 (m, 1H), 2.28-2.08 (m, 1H), 1.75-1.63 (m, 1H), 1.39-1.27 (m, 1H), 1.23-1.16 (m, 3H), 1.00-0.85 (m, 3H).

MS (ESI): m/z 422.0 [M+H]⁺

### Example 316

### (3S,6S)-3-((R)-sec-butyl)-6-phenyl-4-((1R,2R)-2-phenylcyclopropanecarbonyl)-piperazin-2-one (341)

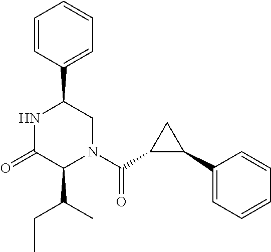

Compound 340 (50 mg, 0.22 mmol) and (1R,2R)-2-phenylcyclopropanecarboxylic acid 54 (35 mg, 0.22 mmol) were coupled according to the procedure described for compound 70 to furnish compound 341 (69 mg, 85% yield) as a colorless solid.

¹H NMR (400 MHz, CDCl₃): δ 7.43-7.10 (m, 10H), 6.05 (s, 1H), 5.15-4.85 (s, 1H), 4.67-4.62 (m, 1H), 4.48-4.21 (m, 1H), 3.45-2.88 (m, 1H), 2.62-2.56 (m, 1H), 2.20-2.14 (m, 1H), 2.02-1.95 (m, 1H), 1.72-1.61 (m, 2H), 1.39-1.24 (m, 2H) 1.24-1.11 (m, 3H), 0.99-0.93 (m, 3H).

MS (ESI): m/z 377.0 [M+H]⁺

### Example 317

### (3S,6S)-4-(5-(4-fluorophenyl)isoxazole-3-carbonyl)-3-neopentyl-6-phenylpiperazin-2-one (342)

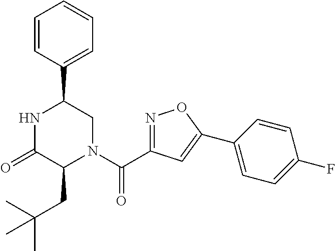

### Step 1: Synthesis of (3S,6S)-3-(2,2-Dimethyl-propyl)-6-phenyl-piperazin-2-one (343)

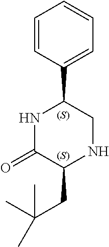

Synthesized from Amino-phenyl-acetic acid (2.6 g, 1.71 mmol) and 2-Amino-4,4-dimethyl-pentanoic acid methyl ester (200 mg, 1.26 mmol) by the method described for the compound 7 (Scheme II) to afford the product 343 (250 mg, overall yield: 5.92%).

¹H NMR (400 MHz, DMSO-d6) 7.89 (s, 1H), 7.25-7.38 (m, 5H), 4.48 (s, 1H), 3.26 (d, J=7.6 Hz, 1H), 3.05 (dd, J=4.4, 13.2 Hz, 1H), 2.72 (dd, J=5.2, 13.2 Hz, 1H), 2.19 (s, 1H), 1.82-1.86 (m, 1H), 1.383-1.44 (m, 1H), 0.93 (s, 9H)

MS (ESI): m/z 247.1 [M+H]⁺

### Step 2

Compound 343 (49 mg, 0.20 mmol) and 5-(4-Fluoro-phenyl)-isoxazole-3-carboxylic acid (41 mg, 0.20 mmol) were coupled according to the procedure described for compound 70 to furnish compound 342 (68 mg, 78% yield) as a white solid.

¹H NMR (CDCl₃) δ 7.82-7.79 (m, 2H), 7.45-7.38 (m, 5H), 7.22-7.17 (m, 2H), 6.89 (s, 0.7H), 6.88 (s, 0.3H), 6.23 (bs, 1H), 5.51 (d, J=9.6 Hz, 1H), 4.97-4.91 (m, 2H), 3.45 (m, 1H), 2.09-1.90 (m, 2H), 1.05 (s, 9H);

MS (ESI): m/z 436.2 [M+H]⁺

### Example 318

### (3S,6S)-3-neopentyl-6-phenyl-4-((1R,2R)-2-phenylcyclopropanecarbonyl)piperazin-2-one (344)

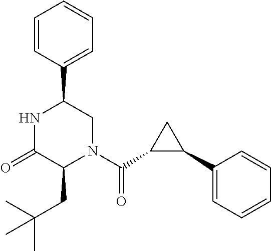

Compound 343 (49 mg, 0.20 mmol) and (1R,2R)-2-Phenyl-cyclopropanecarboxylic acid 54 (32 mg, 0.20 mmol) were coupled according to the procedure described for compound 70 to furnish compound 344 (58 mg, 74% yield) as a white solid.

¹H NMR (CDCl₃) δ 7.42-7.27 (m, 7H), 7.26-7.08 (m, 4H), 6.02 (s, 0.6H), 5.87 (s, 0.4H), 6.23 (bs, 1H), 5.46 (d, J=10.4 Hz, 1H), 4.80-4.68 (m, 2H), 4.16 (m, 1H), 3.45 (m, 1H), 2.11-1.89 (m, 2H), 1.85-1.68 (m, 3H), 1.42 (m, 1H), 1.04 (s, 4H), 1.00 (s, 5H).

MS (ESI): m/z 391.2 [M+H]⁺

### Example 319

### (3S,6S)-6-(4-fluorophenyl)-4-(5-(4-fluorophenyl)isoxazole-3-carbonyl)-3-isobutyl-piperazin-2-one (345)

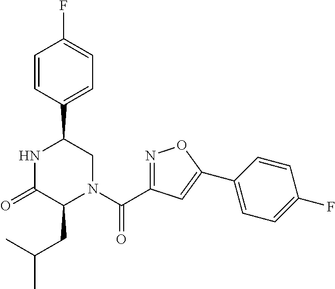

### Step 1: Synthesis of (3S,6S)-6-(4-Fluoro-phenyl)-3-isobutyl-piperazin-2-one (346)

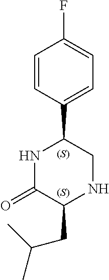

Synthesized from (S)-Amino-(4-fluoro-phenyl)-acetic acid (10 g, 0.06 mol) and Hydrochloride salt of L-Leucine methyl ester (1.56 g, 10.67 mmol) by the method described for the compound 7 (Scheme II) to afford the product 346 (420 mg, overall yield: 2.8%) as a white solid.

¹H NMR (400 MHz, DMSO-d6) 7.96 (s, 1H), 7.37 (dd, J=2.8 Hz, 6.4 Hz, 2H), 7.23 (dd, J=9.2 Hz, 11.2 Hz, 2H), 4.54 (d, J=2.4 Hz, 1H), 3.26 (dd, J=3.6 Hz, 10 Hz, 1H), 3.10 (dd, J=4.4 Hz, 13.2 Hz, 1H), 2.79 (dd, J=4.8 Hz, 12.8 Hz, 1H), 2.45 (s, 1H), 1.80-1.84 (m, 1H), 1.53-1.70 (m, 2H), 0.95 (d, J=6.8 Hz, 3H), 0.91 (d, J=6.4 Hz, 3H)

MS (ESI): m/z 251.1 [M+H]⁺

### Step 2

Compound 346 (50 mg, 0.20 mmol) and 5-(4-Fluoro-phenyl)-isoxazole-3-carboxylic acid (41 mg, 0.20 mmol) were coupled according to the procedure described for compound 70 to furnish compound 345 (68 mg, 78% yield) as a white solid.

¹H NMR (CDCl₃) δ 7.82-7.78 (m, 2H), 7.40-7.37 (m, 2H), 7.22-7.18 (m, 2H), 7.14-7.10 (m, 2H), 6.90 (s, 0.7H), 6.81 (s, 0.3H), 6.06 (bs, 0.7H), 6.02 (bs, 0.3H), 5.47-5.44 (m, 0.3H), 5.39-5.36 (m, 0.7H), 4.98-4.76 (m, 2H), 3.31 (dd, J=10.0, 13.6 Hz, 0.7H), 3.06 (dd, J=10.8, 13.6 Hz, 0.3H), 2.01-1.92 (m, 1H), 1.89-1.73 (m, 2H), 1.11 (d, J=6.4 Hz, 2.1H), 1.00 (d, J=6.8 Hz, 2.1H), 0.96-0.82 (m, 1.8H).

MS (ESI): m/z 440.2 [M+H]⁺

### Example 320

### (3S,6S)-6-(4-fluorophenyl)-3-isobutyl-4-((1R,2R)-2-phenylcyclopropanecarbonyl)-piperazin-2-one (347)

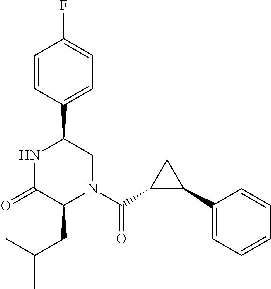

Compound 346 (50 mg, 0.20 mmol) and (1R,2R)-2-Phenyl-cyclopropanecarboxylic acid 54 (32 mg, 0.20 mmol) were coupled according to the procedure described for compound 70 to furnish compound 347 (70 mg, 89% yield) as a white solid.

¹H NMR (CDCl₃) δ 7.34-7.28 (m, 4H), 7.24-7.20 (m, 2H), 7.12-7.08 (m, 4H), 5.88 (bs, 1H), 5.32 (dd, J=3.6, 9.6 Hz, 0.4H), 4.78 (dd, J=3.6 Hz, 14.0 Hz, 0.6H), 4.70-4.63 (m, 1.6H), 4.14 (dd, J=4.0, 14.0 Hz, 0.4H), 3.33 (dd, J=11.2, 14.8 Hz, 0.4H), 2.88-2.81 (m, 0.6H), 2.61-2.55 (m, 1H), 2.00-1.66 (m, 7H), 1.42-1.33 (m, 1H), 1.05-0.96 (m, 6H).

MS (ESI): m/z 395.2 [M+H]⁺

### Example 321

### (3S,6S)-4-(5-(4-fluorophenyl)isoxazole-3-carbonyl)-3-isobutyl-6-(p-tolyl)piperazin-2-one (348)

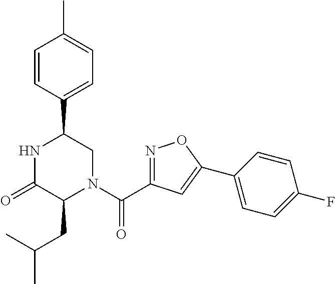

### Step 1: Synthesis of (3S,6S)-3-Isobutyl-6-p-tolyl-piperazin-2-one (349)

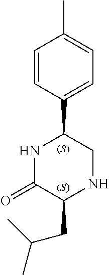

Synthesized from Amino-p-tolyl-acetic acid (5.0 g, 30 mmol) and Hydrochloride salt of L-Leucine methyl ester (4.37 g, 24.3 mmol) by the method described for the compound 7 (Scheme II) to afford the product 349 (1.44 g, overall yield: 19.5%). Step 2: Compound 349 (50 mg, 0.20 mmol) and 5-(4-Fluoro-phenyl)-isoxazole-3-carboxylic acid (41 mg, 0.20 mmol) were coupled according to the procedure described for compound 70 to furnish compound 348 (77 mg, 88% yield) as a white solid.

¹H NMR (CDCl₃) δ 7.82-7.78 (m, 2H), 7.28-7.18 (m, 6H), 6.89 (s, 0.7H), 6.81 (s, 0.3H), 6.01 (bs, 0.7H), 5.97 (bs, 0.3H), 5.45-5.36 (m, 1H), 4.94-4.73 (m, 2H), 3.33 (dd, J=10.4, 14.0 Hz, 0.7H), 3.07 (dd, J=11.2, 13.6 Hz, 0.3H), 2.38 (s, 0.9H), 2.37 (s, 2.1H), 2.05-1.68 (m, 3H), 1.17-0.81 (m, 6H).

MS (ESI): m/z 436.2 [M+H]⁺

### Example 322

### (3S,6S)-4-(1-(4-chlorophenyl)-1H-1,2,3-triazole-4-carbonyl)-3,6-diisobutylpiperazin-2-one (350)

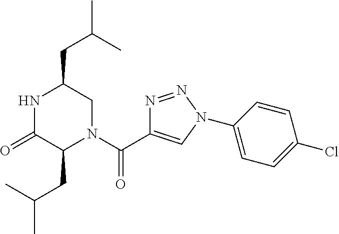

Compound 7 (43 mg, 0.20 mmol) and 1-(4-Chloro-phenyl)-1H-[1,2,3]triazole-4-carboxylic acid (45 mg, 0.20 mmol) were coupled according to the procedure described for compound 70 to furnish compound 350 (68 mg, 81% yield) as a white solid.

¹H NMR (CDCl₃) δ 8.59 (s, 0.8H), 8.51 (s, 0.2H), 7.72-7.69 (m, 2H), 7.56-7.53 (m, 2H), 6.08 (t, J=6.8 Hz, 0.2H), 5.96 (bs, 1H), 5.68 (dd, J=4.0, 14.0 Hz, 0.8H), 5.32 (m, 1H), 4.80 (dd, J=4.0, 13.2 Hz, 0.2 Hz), 3.81 (m, 0.8H), 3.70 (m, 0.2H), 3.17 (dd, J=10.8, 14.0 Hz, 0.8H), 2.87 (dd, J=10.8, 13.2 Hz, 0.2H), 1.97-1.68 (m, 4H), 1.41 (m, 2H), 1.09-0.86 (m, 12H).

MS (ESI): m/z 418.2 [M+H]⁺

### Example 323

### (3S,6S)-4-(1-(4-fluorophenyl)-1H-1,2,3-triazole-4-carbonyl)-3,6-diisobutylpiperazin-2-one (351)

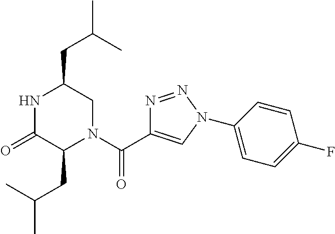

Compound 7 (43 mg, 0.20 mmol) and 1-(4-fluoro-phenyl)-1H-[1,2,3]triazole-4-carboxylic acid (45 mg, 0.20 mmol) were coupled according to the procedure described for compound 70 to furnish compound 351 (69 mg, 86% yield) as a white solid.

¹H NMR (CDCl₃) δ 8.56 (s, 0.8H), 8.49 (s, 0.2H), 7.74 (m, 2H), 7.28-7.24 (m, 2H), 6.09 (t, J=6.8 Hz, 0.2H), 5.98 (bs, 1H), 5.69 (dd, J=4.0, 14.0 Hz, 0.8H), 5.33 (m, 1H), 4.80 (dd, J=4.4, 13.6 Hz, 0.2 Hz), 3.81 (m, 0.8H), 3.70 (m, 0.2H), 3.17 (dd, J=11.2, 14.0 Hz, 0.8H), 2.87 (dd, J=11.2, 13.6 Hz, 0.2H), 1.97-1.68 (m, 4H), 1.41 (m, 2H), 1.09-0.86 (m, 12H).

MS (ESI): m/z 402.2 [M+H]⁺

### Example 324

### (3S,6S)-6-(2-chlorophenyl)-4-(5-(4-fluorophenyl)isoxazole-3-carbonyl)-3-isobutyl-piperazin-2-one (352)

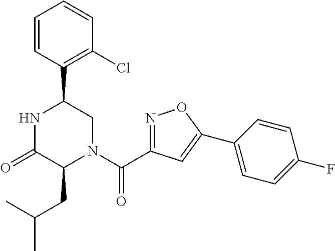

### Step 1: Synthesis of (3S,6S)-6-(2-Chloro-phenyl)-3-isobutyl-piperazin-2-one (353)

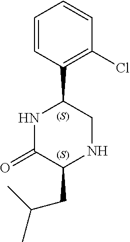

Synthesized from (S)-Amino-(2-chloro-phenyl)-acetic acid (10 g, 0.05 mol) and Hydrochloride salt of L-Leucine methyl ester (7 g, 0.03 mol) by the method described for the compound 7 (Scheme II) to afford the product 353 (1.17 g, overall yield: 8.8%) as a white solid.

¹H NMR (400 MHz, DMSO-d6): δ 7.87 (s, 1H), 7.37-7.44 (m, 2H), 7.29-7.37 (m, 3H), 4.80 (d, J=3.2 Hz, 1H), 3.23 (dd, J=3.2, 10.0 Hz, 1H), 3.13 (dd, J=4.4, 12.8 Hz, 1H), 2.80 (dd, J=3.2, 12.4 Hz, 1H), 2.33-2.40 (m, 1H), 1.65 (dt, J=3.6, 13.2 Hz, 1H), 1.48 (dt, J=3.6, 6.8 Hz, 1H), 0.90 (d, J=8.4 Hz, 3H), 0.85 (d, J=8.4 Hz, 3H).

MS (ESI): m/z 267.0 [M+H]⁺

### Step 2

Compound 353 (53 mg, 0.20 mmol) and 5-(4-Fluoro-phenyl)-isoxazole-3-carboxylic acid (41 mg, 0.20 mmol) were coupled according to the procedure described for compound 70 to furnish compound 352 (69 mg, 86% yield) as a white solid.

¹H NMR (CDCl₃) δ 7.83-7.79 (m, 2H), 7.51-7.28 (m 4H), 7.22-7.18 (m, 2H), 6.87 (s, 0.8H), 6.84 (s, 0.2H), 5.93 (bs, 0.8H), 5.92 (bs, 0.2H), 5.52 (m, 0.2H), 5.43 (m, 0.8H), 5.27 (dd, J=4.8, 11.2 Hz, 1H), 5.11-5.06 (m, 2H), 3.33 (dd, J=6.8, 14.0 Hz, 0.8H), 3.05 (dd, J=6.8, 14.0 Hz, 0.8H), 2.01-1.84 (m, 2H), 1.81-1.72 (m, 1H), 1.10 (d, J=6.4 Hz, 2.4H), 1.01 (d, J=6.8 Hz, 2.4H), 0.97-0.84 (m, 1.2H).

MS (ESI): m/z 456.2 [M+H]⁺

### Example 325

### (3S,6S)-6-(2-chlorophenyl)-3-isobutyl-4-((1R,2R)-2-phenylcyclopropanecarbonyl)-piperazin-2-one (354)

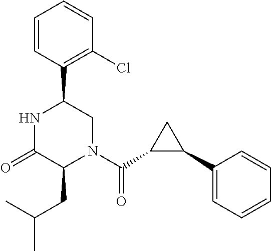

Compound 353 (53 mg, 0.20 mmol) and (R,2R)-2-Phenyl-cyclopropanecarboxylic acid 54 (32 mg, 0.20 mmol) were coupled according to the procedure described for compound 70 to furnish compound 354 (70 mg, 91% yield) as a white solid.

¹H NMR (CDCl₃) δ 7.48-7.20 (m, 5H), 7.11 (d, J=7.6 Hz, 2H), 5.81 (m, 1H), 5.35-4.99 (m, 2H), 4.69 (m, 1H), 4.40 (dm, J=14.4 Hz, 1H), 3.17 (dd, J=10.8, 14.4 Hz, 1H), 2.86-2.79 (m, 0.5H), 2.63-2.52 (m, 1.5H), 2.20-2.15 (m, 1H), 2.0-6.70 (m, 2H), 1.40-1.34 (m, 1H), 1.06-0.94 (m, 6H).

MS (ESI): m/z 411.2 [M+H]⁺

### Example 326

### (3S,6S)-6-(2-fluorophenyl)-4-(5-(4-fluorophenyl)isoxazole-3-carbonyl)-3-isobutyl-piperazin-2-one (355)

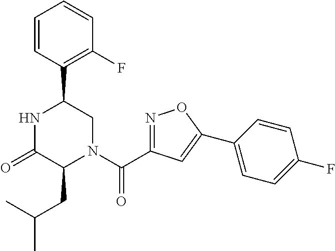

### Step 1: Synthesis of (3S,6S)-6-(2-Fluoro-phenyl)-3-isobutyl-piperazin-2-one (356)

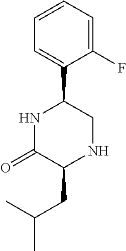

Synthesized from Amino-(2-fluoro-phenyl)-acetic acid (5.0 g, 29.58 mmol) and Hydrochloride salt of L-Leucine methyl ester (7.8 g, 42.95 mmol) by the method described for the compound 7 (Scheme II) to afford the product 356 (660.09 mg, overall yield: 8.9%).

¹H NMR (400 MHz, CDCl₃) δ 7.28-7.36 (m, 2H), 7.17-7.21 (m, 1H), 7.04-7.09 (m, 1H), 6.14 (s, 1H), 4.96 (dd, J₁=7.8 Hz, J₂=4.0 Hz, 1H), 3.50 (dd, J₁=10.8 Hz, J₂=3.6 Hz, 1H), 3.32 (dd, J₁=10.8 Hz, J₂=3.6 Hz, 1H), 3.07 (dd, J₁=13.2 Hz, J₂=3.6 Hz, 1H), 1.85-1.91 (m, 1H), 1.75-1.81 (m, 1H), 1.51-1.58 (m, 2H), 0.91-0.96 (m, 6H).

MS (ESI): m/z 251.1 [M+H]⁺

### Step 2

Compound 356 (50 mg, 0.20 mmol) and 5-(4-Fluoro-phenyl)-isoxazole-3-carboxylic acid (41 mg, 0.20 mmol) were coupled according to the procedure described for compound 70 to furnish compound 355 (69 mg, 86% yield) as a white solid.

¹H NMR (CDCl₃): δ 7.83-7.78 (m, 2H), 7.43-7.34 (m, 2H), 7.25-7.09 (m, 4H), 6.89 (s, 0.8H), 6.82 (s, 0.2H), 5.90 (bs, 0.8H), 5.88 (bs, 0.2H), 5.50-5.45 (m, 0.2H), 5.41 (dd, J=4.8, 10.0 Hz, 0.8H), 5.17-5.02 (m, 2H), 3.49 (dd, J=11.2, 14.4 Hz, 0.8H), 3.23 (dd, J=11.2, 14.4 Hz, 0.2H), 2.01-1.86 (m, 2H), 1.82-1.70 (m, 1H), 1.11 (d, J=6.4 Hz, 2.4H), 1.01 (d, J=6.8 Hz, 2.4H), 0.96 (d, J=6.4 Hz, 0.6H), 0.83 (d, J=6.4 Hz, 0.6H).

MS (ESI): m/z 440.2 [M+H]⁺

### Example 327

### (3S,6S)-6-(2-fluorophenyl)-3-isobutyl-4-((1R,2R)-2-phenylcyclopropanecarbonyl)-piperazin-2-one (357)

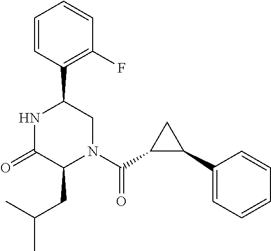

Compound 356 (53 mg, 0.20 mmol) and (R,2R)-2-Phenyl-cyclopropanecarboxylic acid 54 (32 mg, 0.20 mmol) were coupled according to the procedure described for compound 70 to furnish compound 357 (71 mg, 90% yield) as a white solid.

¹H NMR (CDCl₃): δ 7.43-7.27 (m, 4H), 7.23-7.20 (m, 2H), 7.14-7.08 (m, 3H), 5.87 (d, J=6.4 Hz, 1H), 5.33 (dd, J=4.0, 10.4 Hz, 0.5H), 5.08 (dd, J=4.4, 11.2 Hz, 0.5H) 4.98-4.87 (m, 1H), 4.70-4.67 (m, 0.5H), 4.30 (dd, J=4.0, 13.2 Hz, 0.5H), 3.33 (dd, J=6.8, 14.4 Hz, 0.5H), 3.00 (dd, J=6.8, 14.4 Hz, 0.5H), 2.59 (m, 1H), 2.07 (m, 1H), 2.00-1.82 (m, 3H), 1.76-1.68 (m, 1H), 1.37 (m, 1H), 1.05-0.95 (m, 6H).

MS (ESI): m/z 395.2 [M+H]⁺

### Example 328

### (3S,6S)-6-(4-chlorophenyl)-4-(5-(4-fluorophenyl)isoxazole-3-carbonyl)-3-isobutyl-piperazin-2-one(358)

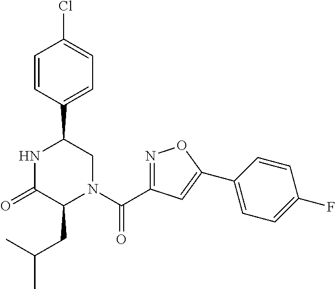

### Step 1: (3S,6S)-6-(4-Chloro-phenyl)-3-isobutyl-piperazin-2-one (359)

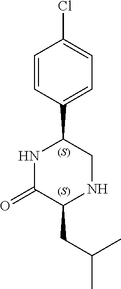

Synthesized from Amino-(4-chloro-phenyl)-acetic acid (10.0 g, 53.88 mmol) and Hydrochloride salt of L-Leucine methyl ester (13.9 g, 76.51 mmol) by the method described for the compound 7 (Scheme II) to afford the product 359 (445 mg, overall yield: 3.1%) as a white solid.

¹H NMR (400 MHz, CDCl3): δ 7.34-7.37 (m, 2H), 7.20-7.25 (m, 2H), 5.94 (s, 1H), 4.60 (dd, J₁=4.8 Hz, J₂=7.8 Hz, 1H), 3.51 (dd, J₁=3.2 Hz, J₂=10.4 Hz, 1H), 3.26 (dd, J₁=4.8 Hz, J₂=13.6 Hz, 1H), 2.94-2.99 (m, 1H), 1.75-1.88 (m, 3H), 1.55-1.61 (m, 1H), 0.94 (dd, J₁=6.4 Hz, J₂=12.8 Hz, 6H).

MS (ESI): m/z 267.0 [M+H]⁺

### Step 2

Compound 359 (53 mg, 0.20 mmol) and 5-(4-Fluoro-phenyl)-isoxazole-3-carboxylic acid (41 mg, 0.20 mmol) were coupled according to the procedure described for compound 70 to furnish compound 358 (73 mg, 85% yield) as a white solid.

¹H NMR (CDCl₃): δ 7.82-7.78 (m, 2H), 7.42-7.34 (m, 4H), 7.20 (m, 2H), 6.90 (s, 0.7H), 6.81 (s, 0.3H), 5.93 (bs, 0.7H), 5.89 (bs, 0.3H), 5.49-5.45 (m, 0.3H), 5.39 (dd, J=4.4, 14.4 Hz, 0.7H), 4.99-4.76 (m, 2H), 3.30 (dd, J=10.8, 14.0 Hz, 0.7H), 3.05 (dd, J=10.8, 13.6 Hz, 0.3H), 2.01-1.70 (m, 3H), 1.02 (d, J=6.4 Hz, 2.1H), 1.00 (d, J=6.8 Hz, 2.1H), 0.97-0.82 (m, 1.8H).

MS (ESI): m/z 456.2 [M+H]⁺

### Example 329

### (3S,6S)-6-(4-chlorophenyl)-3-isobutyl-4-((1R,2R)-2-phenylcyclopropanecarbonyl)-piperazin-2-one (360)

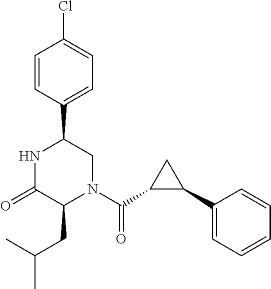

Compound 359 (53 mg, 0.20 mmol) and (R,2R)-2-Phenyl-cyclopropanecarboxylic acid 54 (32 mg, 0.20 mmol) were coupled according to the procedure described for compound 70 to furnish compound 360 (64 mg, 83% yield) as a white solid.

¹H NMR (CDCl₃): δ 5.36-5.30 (m, 0.5H), 4.81-4.62 (m, 2H), 4.15 (m, 0.5H), 3.32 (dd, J=10.8, 14.0 Hz, 0.5H), 2.84-2.79 (m, 0.5H), 2.57 (m, 1H), 1.98-1.76 (m, 3H), 1.75-1.68 (m, 1H), 1.35 (m, 1.0H), 1.05-0.96 (m, 6H).

MS (ESI): m/z 411.2 [M+H]⁺

### Example 330

### (3S,6S)-4-(5-(4-fluorophenyl)isoxazole-3-carbonyl)-3-isobutyl-6-(o-tolyl)piperazin-2-one (361)

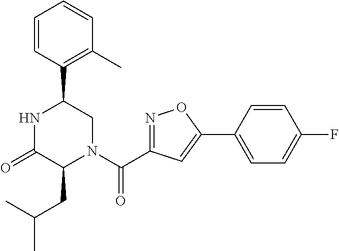

### Step 1: Synthesis of (3S,6S)-3-Isobutyl-6-o-tolyl-piperazin-2-one (362)

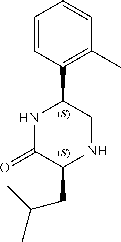

Synthesized from (S)-Amino-o-tolyl-acetic acid (4 g, 24.2 mmol) and Hydrochloride salt of L-Leucine methyl ester (3.3 g, 22.9 mmol) by the method described for the compound 7 (Scheme II) to afford the product 362 (1.2 g, overall yield: 20.1%) as a white solid.

¹H NMR (400 MHz, DMSO-d6): 7.67 (s, 1H), 7.18 (dd, J=2.0, 4.0 Hz, 2H), 7.11 (dd, J=3.2, 4.8 Hz, 2H), 4.66 (m, 1H), 3.17 (dd, J=3.6, 10 Hz, 1H), 3.01 (dd, J=4.4, 13.2 Hz, 1H), 2.63 (dd, J=5.6, 13.2 Hz, 1H), 2.29-2.31 (m, 1H), 2.24 (s, 3H), 1.71-1.77 (m, 1H), 1.43-1.62 (m, 2H), 0.86 (d, J=6.8 Hz, 3H), 0.82 (d, J=6.8 Hz, 3H).

MS (ESI): m/z 247.1 [M+H]⁺

### Step 2

(3S,6S)-3-isobutyl-6-(o-tolyl)piperazin-2-one 362 (50 mg, 0.2 mmol) and 5-(4-fluorophenyl)isoxazole-3-carboxylic acid (42 mg, 0.2 mmol) were coupled according to the procedure described for compound 70 to furnish compound 361 (77 mg, 87% yield) as colorless solid.

¹H NMR (400 MHz, CDCl₃): δ 7.82-7.77 (m, 2H), 7.42-7.39 (m, 1H), 7.30-7.17 (m, 5H), 6.89 (s, 1H), 5.90 (s, 1H), 5.46-5.37 (m, 1H), 5.17-5.02 (m, 1H), 4.98-4.87 (m, 1H), 3.30-2.96 (m, 1H), 2.45 (s, 3H), 2.04-1.71 (m, 3H), 1.12-0.83 (m, 6H).

MS (ESI): m/z 436.1 [M+H]⁺

### Example 331

### (3S,6S)-3-isobutyl-4-((1R,2R)-2-phenylcyclopropanecarbonyl)-6-(o-tolyl)piperazin-2-one (363)

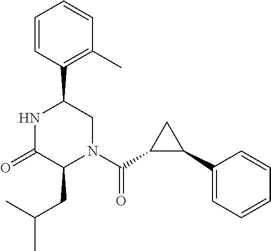

Compound 362 (50 mg, 0.2 mmol) and (1R,2R)-2-phenylcyclopropanecarboxylic acid 54 (33 mg, 0.2 mmol) were coupled according to the procedure described for compound 70 to furnish compound 363 (69 mg, 87% yield) as colorless solid.

¹H NMR (400 MHz, CDCl₃): δ 7.38-7.08 (m, 9H), 5.86-5.84 (m, 1H), 5.33-4.95 (m, 1H), 4.94-4.78 (m, 1H), 4.68-4.15 (m, 1H), 3.31-2.75 (m, 1H), 2.61-2.55 (m, 1H), 2.42 (s, 2H, CH₃, split), 2.36 (s, 1H, CH₃, split), 1.99-1.65 (m, 5H), 1.39-1.31 (m, 1H), 1.05-0.95 (m, 6H).

MS (ESI): m/z 391.1 [M+H]⁺

### Example 332

### (3S,6S)-3-isobutyl-4-((1R,2R)-2-phenylcyclopropanecarbonyl)-6-(4-(trifluoro-methyl)phenyl)piperazin-2-one (364)

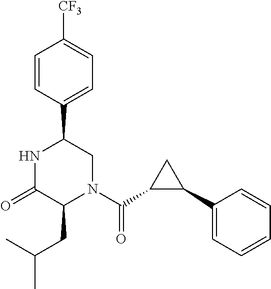

### Step 1: Synthesis of (3S,6S)-3-Isobutyl-6-(4-trifluoromethyl-phenyl)-piperazin-2-one (365)

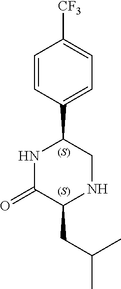

Synthesized from (S)-Amino-(4-trifluoromethyl-phenyl)-acetic acid (3.0 g, 13.70 mmol) and Hydrochloride salt of L-Leucine methyl ester (3.5 g, 19.20 mmol) by the method described for the compound 7 (Scheme II) to afford the product 365 (217 mg, overall yield: 5.3%) as a white solid.

¹H NMR (400 MHz, CDCl3): δ 7.65 (d, J=8.0 Hz, 2H), 7.42 (d, J=8.0 Hz, 2H), 6.07 (s, 1H), 4.70-4.71 (m, 1H), 3.54 (dd, J₁=10.4 Hz, J₂=3.6 Hz, 1H), 3.33 (dd, J₁=13.2 Hz, J₂=4.8 Hz, 1H), 3.02 (dd, J₁=13.2 Hz, J₂=4.4 Hz, 1H), 1.75-1.88 (m, 3H), 1.58-1.62 (m, 1H), 0.97 (d, J=6.4 Hz, 3H), 0.94 (d, J=6.4 Hz, 3H).

MS (ESI): m/z 301.0 [M+H]⁺

### Step 2

Compound 365 (50 mg, 0.2 mmol) and (1R,2R)-2-phenylcyclopropanecarboxylic acid 54 (27 mg, 0.17 mmol) were coupled according to the procedure described for compound 70 to furnish compound 364 (35 mg, 47% yield) as colorless solid.

¹H NMR (400 MHz, CDCl₃): δ 7.82-7.78 (m, 2H), 7.72-7.69 (m, 2H), 7.56-7.54 (m, 2H), 7.22-7.18 (m, 2H), 6.91 (s, 1H), 6.11-6.08 (m, 1H), 5.51-5.37 (m, 1H), 5.04-5.00 (m, 1H), 4.94-4.85 (m, 1H), 3.35-3.04 (m, 1H), 2.01-1.94 (m, 1H), 1.89-1.71 (m, 2H), 1.12-0.82 (m, 6H).

MS (ESI): m/z 490.1 [M+1]⁺

### Example 333

### (3S,6S)-4-(5-(4-fluorophenyl)isoxazole-3-carbonyl)-3-isobutyl-6-(4-(trifluoromethyl)-phenyl)piperazin-2-one (366)

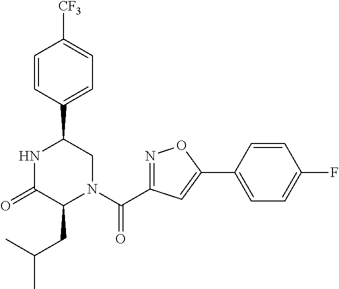

Compound 365 (50 mg, 0.17 mmol) and 5-(4-fluorophenyl)isoxazole-3-carboxylic acid (35 mg, 0.17 mmol) were coupled according to the procedure described for compound 71 to furnish compound 366 (43 mg, 53% yield) as colorless solid.

¹H NMR (400 MHz, CDCl₃): δ 7.69-7.67 (m, 2H), 7.51-7.47 (m, 2H), 7.32-7.20 (m, 3H), 7.12-7.08 (m, 1H), 6.03 (s, 1H), 5.35-4.80 (m, 1H), 4.78-4.72 (m, 1H), 4.69-4.17 (m, 1H), 3.37-2.81 (m, 1H), 2.60-2.55 (m, 1H), 1.98-1.91 (m, 2H), 1.88-1.82 (m, 1H) 1.79-1.60 (m, 2H), 1.40-1.33 (m, 1H), 1.04-0.91 (m, 6H).

MS (ESI): m/z 445.1 [M+1]⁺

### Example 334

### (3S,6S)-6-(3-fluorophenyl)-4-(5-(4-fluorophenyl)isoxazole-3-carbonyl)-3-isobutylpiperazin-2-one (367)

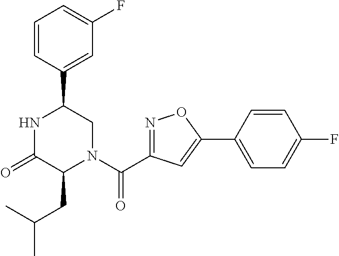

### Step 1: Synthesis of (3S,6S)-6-(3-Fluoro-phenyl)-3-isobutyl-piperazin-2-one (368)

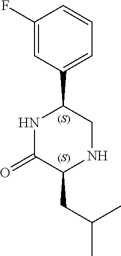

Synthesized from (S)-Amino-(3-fluoro-phenyl)-acetic acid (9 g, 53.21 mmol) and Hydrochloride salt of L-Leucine methyl ester (6.9 g, 37.98 mmol) by the method described for the compound 7 (Scheme 1) to afford the product 368 (478 mg, overall yield: 3.6%) as a white solid.

¹H NMR (400 MHz, CDCl3): δ 7.32-7.38 (m, 1H), 6.99-7.07 (m, 2H), 6.10 (s, 1H), 4.62 (dd, J₁=4.4 Hz, J₂=7.6 Hz, 1H), 3.50 (dd, J₁=4.4 Hz, J₂=13.2 Hz, 1H), 3.00 (dd, J₁=4.8 Hz, J₂=13.2 Hz, 1H), 1.76-1.88 (m, 2H), 1.55-1.60 (m, 2H), 0.95 (dd, J₁=6.4 Hz, J₂=12.8 Hz, 6H).

MS (ESI): m/z 251.0 [M+H]⁺

### Step 2

Compound 368 (50 mg, 0.2 mmol) and 5-(4-fluorophenyl)isoxazole-3-carboxylic acid (41 mg, 0.2 mmol) were coupled according to the procedure described for compound 71 to furnish compound 367 (67 mg, 76% yield) as colorless solid.

¹H NMR (400 MHz, CDCl₃): δ 7.82-7.78 (m, 2H), 7.44-7.37 (m, 1H), 7.26-7.18 (m, 3H), 7.13-7.05 (m, 2H), 6.90-6.81 (m, 1H), 6.12-6.10 (m, 1H), 5.45-5.35 (m, 1H), 5.01-4.77 (m, 2H), 3.35-3.04 (m, 1H), 1.99-1.92 (m, 1H), 1.89-1.08 (m, 1H), 1.78-1.71 (m, 1H), 1.11-0.81 (m, 6H).

MS (ESI): m/z 440.0 [M+H]⁺

### Example 335

### (3S,6S)-6-(3-fluorophenyl)-3-isobutyl-4-((1R,2R)-2-phenylcyclopropanecarbonyl)-piperazin-2-one (369)

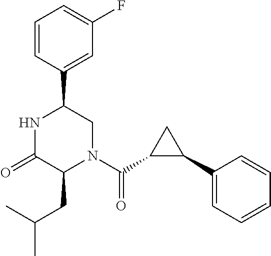

Compound 368 (50 mg, 0.2 mmol) and (1R,2R)-2-phenylcyclopropanecarboxylic acid 54 (33 mg, 0.2 mmol) were coupled according to the procedure described for compound 70 to furnish compound 369 (59 mg, 75% yield) as colorless solid.

¹H NMR (400 MHz, CDCl₃): δ 7.41-7.36 (m, 1H), 7.35-7.18 (m, 3H), 7.12-7.04 (m, 5H), 6.14 (s, 1H), 5.32-4.78 (m, 1H), 4.70-4.15 (m, 2H), 3.36-2.81 (m, 1H), 2.61-2.55 (m, 1H), 2.02-1.64 (m, 5H), 1.41-1.32 (m, 1H), 1.04-0.93 (m, 6H).

MS (ESI): m/z 395.0 [M+1]⁺

### Example 336

### (3S,6S)-6-(2-fluorophenyl)-4-(5-(4-fluorophenyl)-1,2,4-oxadiazole-3-carbonyl)-3-isobutylpiperazin-2-one (370)

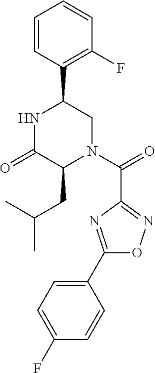

According to the method described for compound 71, compound 356 (60 mg, 0.24 mmol) and 5-(4-fluorophenyl)-1,2,4-oxadiazole-3-carboxylic acid 66 (50 mg, 0.24 mmol) were coupled to give the product 370 as a white solid. (49.5 mg, 47% yield)

¹H NMR (400 MHz, CDCl3): δ 8.25-8.20 (2H, m), 7.42-7.33 (2H, m), 7.28-7.20 (3H, m), 7.10-7.06 (1H, m), 6.23 (1H, s), 5.41-5.37 (1H, m), 5.28-5.24 (1H, m), 4.47-4.43 (1H, m), 3.44 (1H, dd, J=10.8, 14 Hz), 1.98-1.74 (3H, m), 1.11 (3H, d, J=6.4 Hz), 1.00 (3H, d, J=6.4 Hz).

MS (ESI): m/z 441.0 [M+H]⁺.

### Example 337

### (3S,6S)-6-(2-chlorophenyl)-4-(5-(4-fluorophenyl)-1,2,4-oxadiazole-3-carbonyl)-3-isobutylpiperazin-2-one (371)

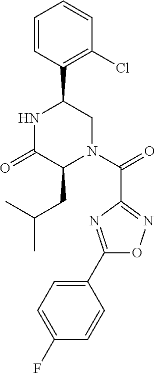

According to the method described for compound 71, compound 353 (64 mg, 0.24 mmol) and 5-(4-fluorophenyl)-1,2,4-oxadiazole-3-carboxylic acid 66 (50 mg, 0.24 mmol) were coupled to give the product 371 as a white solid. (70.3 mg, 64% yield)

¹H NMR (400 MHz, CDCl3): δ 8.24-8.21 (2H, m), 7.49-7.44 (1H, m), 7.38-7.23 (5H, m), 6.18 (1H, s), 5.42-5.39 (1H, m), 5.35-5.31 (1H, m), 4.49 (1H, dd, J=4, 14.4 Hz), 3.29 (1H, dd, J=10.8, 14.4 Hz), 1.99-1.76 (3H, m), 1.11 (3H, d, J=6.4 Hz), 1.00 (3H, d, J=6.4 Hz).

MS (ESI): m/z 458.0 [M+1]⁺.

### Example 338

### (3S,6S)-6-(2-fluorophenyl)-4-(3-(4-fluorophenyl)isoxazole-5-carbonyl)-3-isobutylpiperazin-2-one (372)

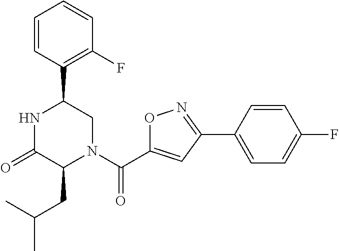

Compound 356 (50 mg, 0.2 mmol) and 3-(4-fluorophenyl)isoxazole-5-carboxylic acid (40 mg, 0.2 mmol) were coupled according to the procedure described for compound 71 to furnish compound 372 (41 mg, 47% yield) as a colorless solid.

¹H NMR (400 MHz, CDCl₃): δ 7.84-7.80 (m, 2H), 7.42-7.36 (m, 2H), 7.24-7.11 (m, 5H), 6.07 (d, J=8.4 Hz, 1H), 5.35-5.14 (m, 1H), 5.06-4.60 (m, 1H), 3.50-3.24 (m, 1H), 3.13-3.07 (m, 1H), 1.95-1.92 (m, 2H), 1.23-1.12 (m, 1H), 1.09-0.87 (m, 6H).

MS (ESI): m/z 440.1 [M+H]⁺

### Example 339

### (3S,6S)-6-(2-fluorophenyl)-4-((1R,2R)-2-(4-fluorophenyl)cyclopropanecarbonyl)-3-isobutylpiperazin-2-one (373)

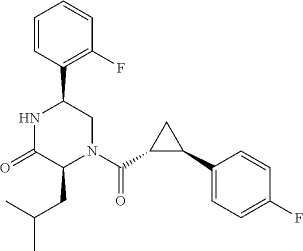

Compound 356 (50 mg, 0.2 mmol) and (1R,2R)-2-(4-fluorophenyl)cyclopropanecarboxylic acid 60 (40 mg, 0.2 mmol) were coupled according to the procedure described for compound 70 to furnish compound 373 (53 mg, 64% yield) as a colorless solid.

¹H NMR (400 MHz, CDCl₃): δ 7.41-7.33 (m, 2H), 7.24-7.17 (m, 1H), 7.12-7.04 (m, 3H), 7.00-6.94 (m, 2H), 6.17 (d, J=22.8 Hz, 1H), 5.30-5.04 (m, 1H), 4.96-4.84 (m, 1H), 4.66-4.25 (m, 1H), 3.36-3.24 (m, 1H), 3.01-2.83 (m, 1H) 2.59-2.52 (m, 1H), 2.07-1.59 (m, 4H), 1.40-1.26 (m, 1H), 1.17-0.92 (m, 6H).

MS (ESI): m/z 413.20 [M+H]⁺

### Example 340

### (3S,6S)-4-((E)-3-(2,4-difluorophenyl)acryloyl)-6-(2-fluorophenyl)-3-isobutylpipera-zin-2-one (374)

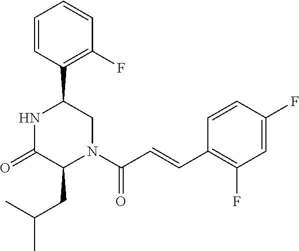

Compound 356 (50 mg, 0.2 mmol) and (E)-3-(2,4-difluorophenyl)acrylic acid (40 mg, 0.2 mmol) were coupled according to the procedure described for compound 71 to furnish compound 374 (47 mg, 56% yield) as colorless solid.

¹H NMR (400 MHz, CDCl₃): δ 7.78-7.74 (m, 1H), 7.50-7.35 (m, 3H), 7.25-6.98 (m, 3H), 6.94-6.84 (m, 2H), 6.08 (d, J=8.0 Hz, 1H), 5.43-5.00 (m, 1H), 4.98-4.61 (m, 1H), 4.27-3.34 (m, 1H), 3.06-2.92 (m, 1H), 1.99-1.77 (m, 2H), 1.33-1.24 (m, 1H), 1.07-0.95 (m, 6H).

MS (ESI): m/z 417.1 [M+1]⁺

### Example 341

### (3S,6S)-6-(2-fluorophenyl)-4-((5-(4-fluorophenyl)isoxazol-3-yl)methyl)-3-isobutyl-piperazin-2-one (375)

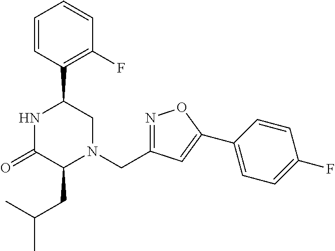

Compound 356 (50 mg, 0.2 mmol) and 5-(4-fluorophenyl)isoxazole-3-carbaldehyde (40 mg, 0.2 mmol) were coupled according to the procedure described for compound 195 to furnish compound 375 (47 mg, 55% yield) as a colorless solid.

¹H NMR (400 MHz, CDCl₃): δ 7.73-7.69 (m, 2H), 7.42-7.38 (m, 1H), 7.32-7.26 (m, 1H), 7.22-7.13 (m, 3H), 7.03-6.98 (m, 1H), 6.28 (s, 1H), 6.00 (s, 1H), 5.21-5.17 (m, 1H), 4.01-3.93 (m, 2H), 3.34-3.31 (m, 1H), 3.06-2.96 (m, 2H), 1.93-1.66 (m, 3H), 0.97-0.87 (m, 6H).

MS (ESI): m/z 426.2 [M+H]⁺

### Example 342

### (3S,6S)-4-(4-fluoro-5-(4-fluorophenyl)isoxazole-3-carbonyl)-3-isobutyl-6-phenyl-piperazin-2-one (376)

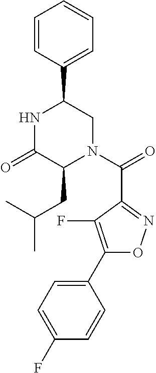

### Step 1: Synthesis of 4-Fluoro-5-(4-fluorophenyl)isoxazole-3-carboxylic acid

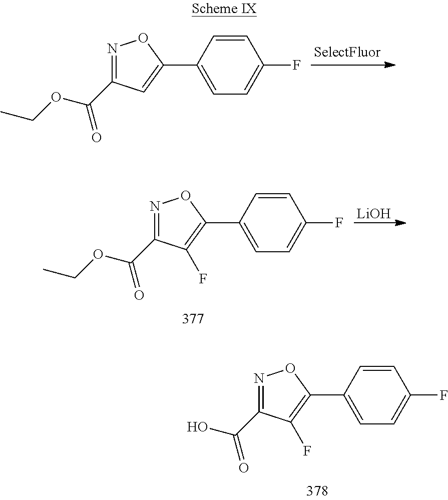

### Step A: Ethyl 4-fluoro-5-(4-fluorophenyl)isoxazole-3-carboxylate (377)

The reaction mixture of ethyl 5-(4-fluorophenyl)isoxazole-3-carboxylate (480.5 mg, 2.04 mmol), selectfluor (760.6 mg, 2.15 mmol) in tetramethylene sulfone (2.06 mL, 21.83 mmol) was heated to 120° C. overnight. After the mixture was cooled to rt, water was added to the mixture. After filtration, solids were collected and purified by column to give the product as a white solid (258 mg, 50%).

¹H NMR (400 MHz, CDCl3): δ 8.04 (2H, dd, J=5.4, 8.6 Hz), 7.22 (2H, d, J=8.4 Hz), 4.50 (2H, q, J=7.2 Hz), 1.45 (3H, t, J=7.2 Hz).

### Step B: 4-Fluoro-5-(4-fluorophenyl)isoxazole-3-carboxylic acid (378)

To a solution of compound 377 (1.0 g, 3.95 mmol) in THF (15 mL) was added LiOH (332 mg, 7.9 mmol) in 15 mL water and 15 mL THF. The reaction was stirred at R.T for 0.5 h. The mixture was adjusted to pH=3-4 with HCl aqueous solution. The mixture was extracted EtOAc (50 mL*2). The combined organic layers were dried Na₂SO₄. The solvent was concentrated to afford desired product 378 (0.833 g, 94% yield) as a white solid.

¹H-NMR (400 MHz, DMSO-d₆) δ 7.89 (dd, J=9.2, 5.6 Hz, 2H), 7.48 (t, J=8.8 Hz, 2H).

### Step 2

According to the method described for compound 89, compound 11 (51 mg, 0.22 mmol) and 4-Fluoro-5-(4-fluoro-phenyl)-isoxazole-3-carboxylic acid 378 (50 mg, 0.22 mmol) were coupled to give the product 376 (63.8 mg, 66% yield) as a colorless solid.

¹H NMR (400 MHz, CDCl3): δ 7.87-7.83 (2H, m), 7.44-7.35 (5H, m), 7.25-7.21 (2H, m), 6.12 (1H, s), 5.38 (1H, dd, J=4, 11 Hz), 4.87 (1H, dd, J=4, 11 Hz), 4.50-4.45 (1H, m), 3.37 (1H, dd, J=11, 14 Hz), 2.04-1.75 (3H, m), 1.11 (3H, d, J=6.4 Hz), 1.02 (3H, d, J=6.4 Hz).

MS (ESI): m/z 440.0 [M+H]⁺.

### Example 343

### (3S,6S)-4-(4-fluoro-5-(4-fluorophenyl)isoxazole-3-carbonyl)-3,6-diisobutylpiperazin-2-one (379)

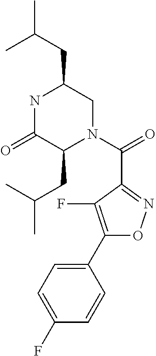

According to the method described for compound 89, compound 7 (50 mg, 0.22 mmol) and 378 (46.6 mg, 0.22 mmol) were coupled to give the product 379 (35.2 mg, 38% yield) as a white solid.

¹H NMR (400 MHz, CDCl3): δ 7.86-7.82 (2H, m), 7.26-7.20 (2H, m), 6.02 (1H, s), 5.28 (1H, dd, J=4, 9.6 Hz), 4.39-4.35 (1H, m), 3.78-3.68 (1H, m), 3.14 (1H, dd, J=11, 14 Hz), 1.93-1.62 (4H, m), 1.43-1.20 (2H, m), 1.09-0.75 (12H, m).

MS (ESI): m/z 420.0 [M+1]⁺.

### Example 344

### (3S,6S)-4-(4-fluoro-5-(4-fluorophenyl)isoxazole-3-carbonyl)-6-(2-fluorophenyl)-3-isobutylpiperazin-2-one (380)

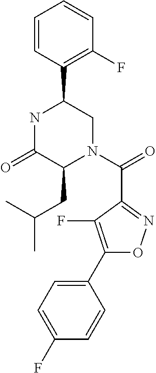

According to the method described for compound 89, compound 356 (55 mg, 0.22 mmol) and 4-Fluoro-5-(4-fluoro-phenyl)-isoxazole-3-carboxylic acid 378 (50 mg, 0.22 mmol) were coupled to give the product 380 (64.4 mg, 64% yiled) as a white solid.

¹H NMR (400 MHz, CDCl3): δ 7.87-7.83 (2H, m), 7.43-7.33 (2H, m), 7.25-7.19 (3H, m), 7.12-7.06 (1H, m), 6.26 (1H, s), 5.38 (1H, dd, J=4, 11 Hz), 5.01-4.98 (1H, m), 4.56-4.51 (1H, m), 3.51-3.45 (1H, m), 2.04-1.71 (3H, m), 1.09 (3H, d, J=6.4 Hz), 1.01 (3H, d, J=6.4 Hz).

MS (ESI): m/z 457.9 [M+1]⁺.

### Example 345

### (3S,6S)-6-cyclopentyl-4-(4-fluoro-5-(4-fluorophenyl)isoxazole-3-carbonyl)-3-isobutylpiperazin-2-one (381)

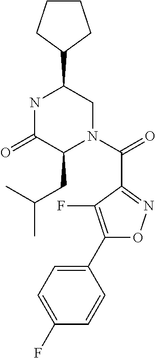

According to the method described for compound 89, compound 17 (49.4 mg, 0.22 mmol) and 4-Fluoro-5-(4-fluoro-phenyl)-isoxazole-3-carboxylic acid 378 (50 mg, 0.22 mmol) were coupled to give the product 381 (51.6 mg, 54% yield) as a white solid.

¹H NMR (400 MHz, CDCl3): δ 7.86-7.81 (2H, m), 7.26-7.19 (2H, m), 6.07 (1H, s), 5.27 (1H, dd, J=4, 9.6 Hz), 4.86-4.81 (1H, m), 4.43-4.39 (1H, m), 3.21-3.15 (1H, m), 1.92-1.57 (10H, m), 1.28-1.15 (2H, m), 1.08 (3H, d, J=6.4 Hz), 0.99 (3H, d, J=6.4 Hz).

MS (ESI): m/z 432.0 [M+1]⁺.

### Example 346

### (3S,6S)-6-cyclohexyl-4-(4-fluoro-5-(4-fluorophenyl)isoxazole-3-carbonyl)-3-isobutylpiperazin-2-one (382)

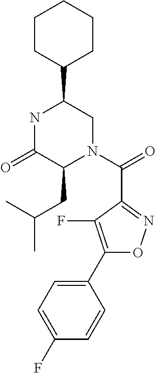

According to the method described for compound 89, compound 18 (52.4 mg, 0.22 mmol) and 378 (50 mg, 0.22 mmol) were coupled to give the product 382 (25.9 mg, 26% yield) as a white solid.

¹H NMR (400 MHz, CDCl3): δ 7.87-7.82 (2H, m), 7.26-7.20 (2H, m), 6.03 (1H, s), 5.30-5.27 (1H, m), 4.86-4.79 (1H, m), 4.42-4.37 (1H, m), 3.29-3.22 (1H, m), 1.90-1.69 (8H, m), 1.59-1.36 (2H, m), 1.29-0.91 (10H, m).

MS (ESI): m/z 446.0 [M+1]⁺.

### Example 347

### (3S,6S)-4-(5-(2,4-difluorophenyl)-4-fluoroisoxazole-3-carbonyl)-3,6-diisobutyl-piperazin-2-one(383)

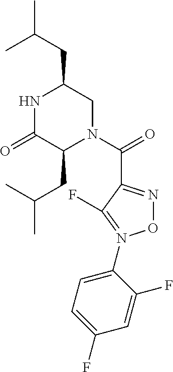

### Step 1: Synthesis of 4-Fluoro-5-(2,4-difluoro-phenyl)-isoxazole-3-carboxylic acid

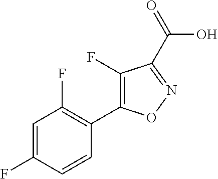

Synthesized from ethyl 5-(2,4-difluorophenyl)isoxazole-3-carboxylate (12 g, 47.4 mmol) by the method described for the compound 378 to afford 384 (1.15 g, 9.98% yield) as a white solid.

¹H NMR (400 MHz, DMSO-d₆): δ 7.84-7.90 (m, 1H), 7.55-7.61 (m, 1H), 7.33 7.38 (m, 1H).

MS (ESI): m/z 244.0 [M+H]⁺

### Step 2

According to the method described for compound 89, compound 7 (44.5 mg, 0.21 mmol) and 384 (50 mg, 0.21 mmol) were coupled to give the product 383 (47.5 mg, 52% yield) as a white solid.

¹H NMR (400 MHz, CDCl3): δ 7.77-7.70 (1H, m), 7.09-6.97 (2H, m), 6.13 (1H, s), 5.29-5.26 (1H, m), 4.84-4.78 (1H, m), 4.36-4.28 (1H, m), 3.18-3.11 (1H, m), 1.92-1.64 (4H, m), 1.43-1.33 (2H, m), 1.09-0.91 (12H, m).

MS (ESI): m/z 438.0 [M+H]⁺.

### Example 348

### (3S,6S)-4-(5-(2,4-difluorophenyl)-4-fluoroisoxazole-3-carbonyl)-3-isobutyl-6-phenylpiperazin-2-one (385)

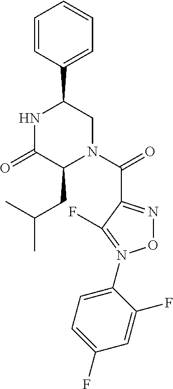

According to the method described for compound 89, compound 11 (48.8 mg, 0.22 mmol) and 384 (50 mg, 0.22 mmol) were coupled to give the product 385 (53.1 mg, 53% yield) as a colorless oil.

¹H NMR (400 MHz, CDCl3): δ 7.78-7.72 (1H, m), 7.47-7.35 (5H, m), 7.10-6.99 (2H, m), 6.04 (1H, s), 5.38 (1H, dd, J=4.4, 11 Hz), 4.88 (1H, dd, J=4.4, 11 Hz), 4.46-4.41 (1H, m), 3.38 (1H, dd, J=11, 14 Hz), 2.04-1.74 (3H, m), 1.11 (3H, d, J=6.4 Hz), 1.02 (3H, d, J=6.4 Hz).

MS (ESI): m/z 457.9 [M+H]⁺.

### Example 349

### (3S,6S)-4-(5-(2,4-difluorophenyl)-4-fluoroisoxazole-3-carbonyl)-6-(2-fluorophenyl)-3-isobutylpiperazin-2-one (386)

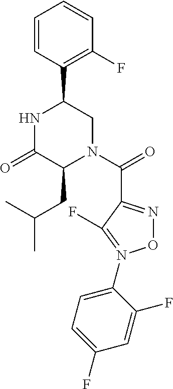

According to the method described for compound 89, compound 356 (52.6 mg, 0.22 mmol) and 384 (50 mg, 0.22 mmol) were coupled to give the product 386 (59.3 mg, 57% yield) as a colorless oil.

¹H NMR (400 MHz, CDCl3): δ 7.78-7.71 (1H, m), 7.42-7.34 (2H, m), 7.24-7.17 (1H, m), 7.12-6.98 (3H, m), 6.21 (1H, s), 5.39 (1H, dd, J=4.4, 11 Hz), 5.16-5.12 (1H, m), 4.5 (1H, dd, J=4.4, 14 Hz), 3.52-3.46 (1H, m), 1.99-1.72 (3H, m), 1.10 (3H, d, J=6.4 Hz), 1.01 (3H, d, J=6.4 Hz).

MS (ESI): m/z 475.9 [M+1]⁺.

### Example 350

### (3S,6S)-4-(5-(2,4-difluorophenyl)-4-fluoroisoxazole-3-carbonyl)-3-isobutyl-6-propylpiperazin-2-one(387)

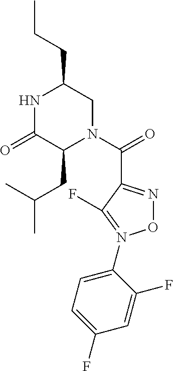

According to the method described for compound 89, compound 16 (41.6 mg, 0.22 mmol) and 384 (50 mg, 0.22 mmol) were coupled to give the product 387 (42.9 mg, 46% yield) as a white solids.

¹H NMR (400 MHz, CDCl3): δ 7.77-7.69 (1H, m), 7.09-6.97 (2H, m), 6.11 (1H, s), 5.29-5.26 (1H, m), 4.84-4.80 (1H, m), 4.36-4.32 (1H, m), 3.20-3.14 (1H, m), 1.93-1.68 (3H, m), 1.56-1.24 (5H, m), 1.09-0.83 (8H, m).

MS (ESI): m/z 424.0 [M+H]⁺.

### Example 351

### (3S,6S)-6-cyclopentyl-4-(5-(2,4-difluorophenyl)-4-fluoroisoxazole-3-carbonyl)-3-isobutylpiperazin-2-ono (388)

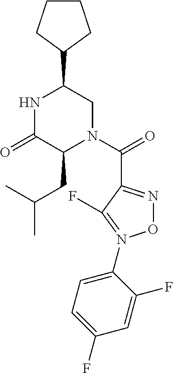

According to the method described for compound 89, compound 17 (47.1 mg, 0.21 mmol) and 384 (50 mg, 0.21 mmol) were coupled to give the product 388 (52 mg, 55% yield) as a white solids.

¹H NMR (400 MHz, CDCl3): δ 7.77-7.70 (1H, m), 7.09-6.99 (2H, m), 6.03 (1H, s), 5.30-5.26 (1H, m), 4.86-4.79 (1H, m), 4.40-4.34 (1H, m), 3.22-3.16 (1H, m), 1.89-1.57 (10H, m), 1.38-1.16 (2H, m), 1.09-0.97 (6H, m).

MS (ESI): m/z 450.0 [M+H]⁺.

### Example 352

### (3S,6S)-6-cyclohexyl-4-(5-(2,4-difluorophenyl)-4-fluoroisoxazole-3-carbonyl)-3-isobutylpiperazin-2-one(389)

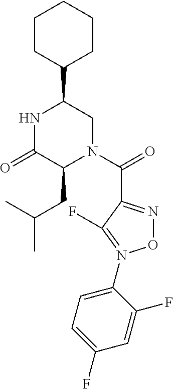

According to the method described for compound 89, compound 18 (50 mg, 0.21 mmol) and 384 (50 mg, 0.21 mmol) were coupled to give the product 389 (21.8 mg, 22% yield) as a colorless oil.

¹H NMR (400 MHz, CDCl3): δ 7.77-7.72 (1H, m), 7.09-6.99 (2H, m), 5.97 (1H, s), 5.28 (1H, dd, J=4, 9.6 Hz), 4.84-4.76 (1H, m), 4.38-4.33 (1H, m), 3.26 (1H, dd, J=11.2, 13.8 Hz), 1.94-1.62 (8H, m), 1.59-1.13 (6H, m), 1.09-0.98 (6H, m).

MS (ESI): m/z 463.9 [M+H]⁺.

### Example 353

### (3S,6S)-4-(5-(3,4-difluorophenyl)-4-fluoroisoxazole-3-carbonyl)-3-isobutyl-6-phenylpiperazin-2-one(390)

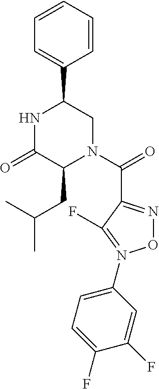

### Step 1: Synthesis of 5-(3,4-difluorophenyl)-4-fluoroisoxazole-3-carboxylic acid (391)

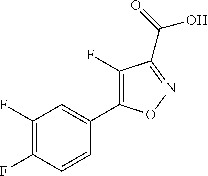

Synthesized from ethyl 5-(3,4-difluorophenyl)isoxazole-3-carboxylate (8.6 g, 33.95 mmol) by the method described for the compound 378 to afford 391 (1.5 g, 2.57% overall yield) as a white solid.

¹HNMR (400 MHz, DMSO-d6): δ 7.85-7.90 (m, 1H), 7.67-7.71 (m, 2H).

MS (ESI): m/z 244.0 [M+H]⁺

### Step 2

According to the method described for compound 89, compound 11 (48.8 mg, 0.21 mmol) and 391 (50 mg, 0.21 mmol) were coupled to give the product 390 (13.8 mg, 14% yield) as colorless oil.

¹H NMR (400 MHz, CDCl3): δ 7.70-7.60 (2H, m), 7.43-6.31 (6H, m), 6.15 (1H, s), 5.36 (1H, dd, J=4, 10 Hz), 4.87 (1H, dd, J=4, 10.8 Hz), 4.48-4.44 (1H, m), 3.38 (1H, dd, J=11.2, 14.4 Hz), 2.02-1.74 (3H, m), 1.11 (3H, d, J=6.8 Hz), 1.01 (3H, d, J=6.8 Hz).

MS (ESI): m/z 458.0 [M+H]⁺.

### Example 354

### (3S,6S)-4-(5-(3,4-difluorophenyl)-4-fluoroisoxazole-3-carbonyl)-6-(2-fluorophenyl)-3-isobutylpiperazin-2-one (392)

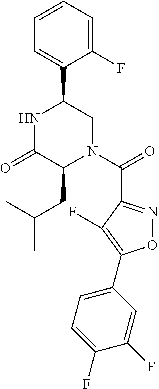

According to the method described for compound 89, compound 356 (52.6 mg, 0.21 mmol) and 391 (50 mg, 0.21 mmol) were coupled to give the product 392 (48.8 mg, 49% yield) as a colorless oil.

¹H NMR (400 MHz, CDCl3): δ 7.70-7.60 (2H, m), 7.41-7.31 (3H, m), 7.24-7.19 (1H, m), 7.17-7.07 (1H, m), 6.41 (1H, s), 5.37 (1H, dd, J=4, 10 Hz), 5.13 (1H, dd, J=4, 10.8 Hz), 4.55-4.50 (1H, m), 3.50-3.44 (1H, m), 1.97-1.65 (3H, m), 1.09 (3H, d, J=6.4 Hz), 1.00 (3H, d, J=6.4 Hz).

MS (ESI): m/z 475.9 [M+H]⁺.

### Example 355

### (3S,6S)-4-(5-(3,4-difluorophenyl)-4-fluoroisoxazole-3-carbonyl)-3-isobutyl-6-propylpiperazin-2-one(393)

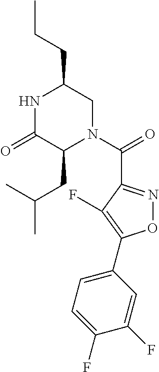

According to the method described for compound 89, compound 16 (41.6 mg, 0.21 mmol) and 391 (50 mg, 0.21 mmol) were coupled to give the product 393 (42 mg, 47% yield) as white solid.

¹H NMR (400 MHz, CDCl₃): δ 7.69-7.59 (2H, m), 7.38-7.31 (1H, m), 6.29 (1H, s), 5.27 (1H, dd, J=4, 9 Hz), 4.85-4.79 (1H, m), 4.39-4.32 (1H, m), 3.16 (1H, dd, J=10.8, 14 Hz), 1.92-1.64 (3H, m), 1.57-1.23 (6H, m), 1.09-0.91 (7H, m).

MS (ESI): m/z 423.9 [M+H]⁺.

### Example 356

### (3S,6S)-4-(5-(3,4-difluorophenyl)-4-fluoroisoxazole-3-carbonyl)-3,6-diisobutyl-piperazin-2-one(394)

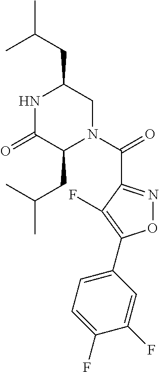

According to the method described for compound 89, compound 7 (44.5 mg, 0.21 mmol) and 391 (50 mg, 0.21 mmol) were coupled to give the product of 394 (29 mg, 32% yield) as white solids.

¹H NMR (400 MHz, CDCl3): δ 7.70-7.60 (2H, m), 7.38-7.31 (1H, m), 6.05 (1H, s), 5.29-5.26 (1H, m), 4.87-4.78 (1H, m), 4.38-4.34 (1H, m), 3.14 (1H, dd, J=11.2, 14.2 Hz), 1.94-1.62 (4H, m), 1.43-1.24 (2H, m), 1.09-0.83 (12H, m).

MS (ESI): m/z 437.9 [M+1]⁺.

### Example 357

### (3S,6S)-6-cyclopentyl-4-(5-(3,4-difluorophenyl)-4-fluoroisoxazole-3-carbonyl)-3-isobutylpiperazin-2-one(395)

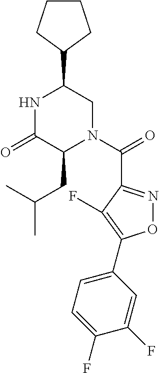

According to the method described for compound 89, compound 17 (47 mg, 0.21 mmol) and 391 (50 mg, 0.21 mmol) were coupled to give the product of 395 (61.7 mg, 65% yield) as white solids.

¹H NMR (400 MHz, CDCl3): δ 7.70-7.60 (2H, m), 7.38-7.31 (1H, m), 6.06 (1H, s), 5.29-5.25 (1H, m), 4.84-4.81 (1H, m), 4.43-4.39 (1H, m), 3.18 (1H, dd, J=11.2, 14 Hz), 1.95-1.54 (10H, m), 1.36-1.18 (2H, m), 1.08 (3H, d, J=6 Hz), 0.99 (3H, d, J=6 Hz).

MS (ESI): m/z 450.0 [M+1]⁺.

### Example 358

### (3S,6S)-6-cyclohexyl-4-(5-(3,4-difluorophenyl)-4-fluoroisoxazole-3-carbonyl)-3-isobutylpiperazin-2-one(396)

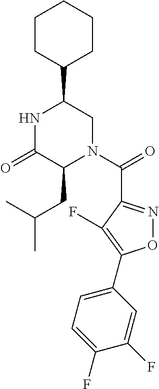

According to the method described for compound 89, compound 18 (50 mg, 0.21 mmol) and 391 (50 mg, 0.21 mmol) were coupled to give the product 396 (32.3 mg, 33% yield) as a white solid.

¹H NMR (400 MHz, CDCl3): δ 7.70-7.60 (2H, m), 7.38-7.30 (1H, m), 6.05 (1H, s), 5.27 (1H, dd, J=4, 9.6 Hz), 4.84-4.78 (1H, m), 4.38 (1H, dd, J=4, 14.2 Hz), 3.26 (1H, dd, J=11.2, 14.2 Hz), 1.94-1.58 (9H, m), 1.52-1.34 (1H, m), 1.30-1.10 (4H, m), 1.08 (3H, d, J=6 Hz), 0.99 (3H, d, J=6 Hz).

MS (ESI): m/z 464.0 [M+1]⁺.

### Example 359

### (3S,6S)-4-(5-(4-fluorophenyl)isoxazole-3-carbonyl)-6-phenyl-3-propylpiperazin-2-one (397)

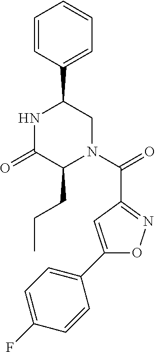

### Step 1: Synthesis of (3S,6S)-6-phenyl-3-propylpiperazin-2-one (398)

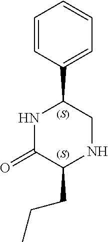

Synthesized from (S)-2-amino-2-phenylacetic acid (30 g, 0.2 mol) and (S)-methyl 2-aminopentanoate hydrochloride (2.6 g, 0.02 mol) by the method described for the compound 7 (Scheme II) to afford the product 398 (1 g, overall yield: 2.3%).

¹H NMR (400 MHz, DMSO-d6): δ 7.87 (s, 1H), 7.26-7.37 (m, 5H), 4.46 (d, J=2.8 Hz, 1H), 3.19 (dd, J=3.6, 8.8 Hz, 1H), 3.07 (dd, J=4.4, 12.8 Hz, 1H), 2.78 (dd, J=4.4, 12.8 Hz, 1H), 1.68-1.76 (m, 1H), 1.56-1.62 (m, 1H), 1.34-1.56 (m, 2H), 0.87-0.98 (m, 3H).

MS (ESI): m/z 219.1 [M+H]⁺

### Step 2

According to the method described for compound 89, compound 398 (100 mg, 0.46 mmol) and 5-(4-Fluoro-phenyl)-isoxazole-3-carboxylic acid (114 mg, 0.55 mmol) were coupled to give the product 397 (75 mg, 40% yield) as pale yellow solid.

¹H NMR (400 MHz, CDCl3): δ 7.81-7.78 (2H, m), 7.44-7.35 (5H, m), 7.22-7.17 (2H, m), 6.89 (1H, s), 6.27 (1H, s), 5.31-5.27 (1H, m), 4.97-4.86 (2H, m), 3.34 (1H, dd, J=10.8, 14 Hz), 2.20-1.68 (2H, m), 1.61-1.41 (2H, m), 1.02-0.90 (3H, m).

MS (ESI): m/z 408.0 [M+H]⁺.

### Example 360

### (3S,6S)-6-phenyl-4-((1R,2R)-2-phenylcyclopropanecarbonyl)-3-propylpiperazin-2-one (399)

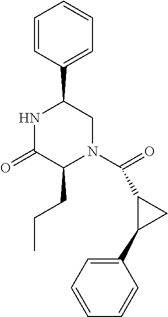

According to the method described for compound 89, compound 398 (100 mg, 0.46 mmol) and 54 (89 mg, 0.55 mmol) were coupled to give the product 399 (24.1 mg, 14% yield) as a white solid.

¹H NMR (400 MHz, CDCl3): δ 7.43-7.10 (10H, m), 6.05 (1H, s), 4.84-4.80 (1H, m), 4.69-4.62 (2H, m), 4.20-4.09 (1H, m), 2.88-2.82 (1H, m), 2.59-2.56 (1H, m), 2.12-1.90 (3H, m), 1.57-1.30 (3H, m), 1.05-0.93 (3H, m).

MS (ESI): m/z 363.0 [M+H]⁺.

### Example 361

### (3S,6S)-4-(5-(4-fluorophenyl)isoxazole-3-carbonyl)-3-isopropyl-6-phenylpiperazin-2-one (400)

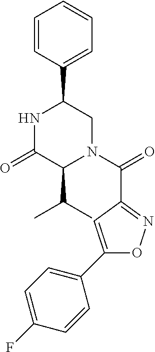

### Step 1: Synthesis of (3S,6S)-3-isopropyl-6-phenylpiperazin-2-one (401)

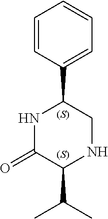

Synthesized from (S)-2-amino-2-phenylacetic acid (30.0 g, 0.19 mol) and (S)-methyl 2-amino-3-methylbutanoate hydrochloride (4.6 g, 0.035 mol) by the method described for the compound 7 (Scheme II) to afford the product 401 (1.2 g, overall yield: 2.9%).

¹H NMR (400 MHz, DMSO-d₆): δ 7.95 (d, J=2.4 Hz, 1H), 7.29-7.35 (m, 4H), 7.22-7.26 (m, 1H), 4.45 (dd, J=3.2, 6.8 Hz, 1H), 3.32 (s, 1H), 3.13 (dt, J=4.4, 3.2 Hz, 2H), 2.90 (dd, J=2.4, 12.4 Hz, 1H), 2.30-2.37 (m, 1H), 0.93 (d, J=7.2 Hz, 3H), 0.88 (d, J=6.8 Hz, 3H).

MS (ESI): m/z 219.0 [M+H]⁺

### Step 2

According to the method described for compound 89, compound 401 (100 mg, 0.46 mmol) and 5-(4-Fluoro-phenyl)-isoxazole-3-carboxylic acid (114 mg, 0.55 mmol) were coupled to give the product 400 (122.3 mg, 65% yield) as colorless oil.

¹H NMR (400 MHz, CDCl3): δ 7.82-7.78 (2H, m), 7.43-7.36 (5H, m), 7.21-7.17 (2H, m), 6.88 (1H, s), 6.35 (1H, s), 5.11-5.04 (1H, m), 4.95-4.84 (2H, m), 3.38 (1H, dd, 10.8, 14 Hz), 2.55-2.46 (1H, m), 1.24 (3H, d, J=7 Hz), 1.09 (3H, d, J=7 Hz).

MS (ESI): m/z 408.0 [M+H]⁺.

### Example 362

### (3S,6S)-3-isopropyl-6-phenyl-4-((1R,2R)-2-phenylcyclopropanecarbonyl)piperazin-2-one (402)

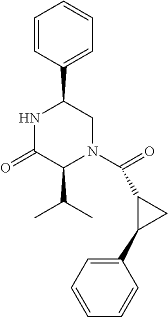

According to the method described for compound 89, compound 401 (100 mg, 0.46 mmol) and 54 (89 mg, 0.55 mmol) were coupled to give the product 402 (82.9 mg, 50% yield) as colorless oil.

¹H NMR (400 MHz, CDCl3): δ 7.43-7.11 (10H, m), 6.21 (1H, s), 4.91-4.86 (1H, m), 4.68-4.62 (1H, m), 4.41-4.37 (1H, m), 2.88 (1H, dd, J=11.2, 13.8 Hz), 2.63-2.55 (1H, m), 2.51-2.40 (1H, m), 2.03-1.96 (1H, m), 1.73-1.66 (1H, m), 1.40-1.33 (1H, m), 1.24 (3H, d, J=7 Hz), 1.08 (3H, d, J=7 Hz).

MS (ESI): m/z 363.0 [M+H]⁺.

### Example 363

### (3S,6S)-4-(5-(4-fluorophenyl)-1,2,4-oxadiazole-3-carbonyl)-3-isopropyl-6-phenyl-piperazin-2-ono (403)

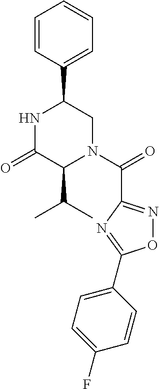

According to the method described for compound 89, compound 401 (52.4 mg, 0.24 mmol) and 66 (50 mg, 0.24 mmol) were coupled to give the product 403 (36.8 mg, 38% yield) as colorless oil.

¹H NMR (400 MHz, CDCl₃): δ 8.24-8.20 (2H, m), 7.44-7.24 (7H, m), 6.16 (1H, s), 5.16-5.14 (1H, m), 4.91-4.87 (1H, m), 4.40-4.34 (1H, m), 3.44 (1H, dd, J=11.2, 14.2 Hz), 2.59-2.41 (1H, m), 1.29-1.11 (6H, m).

MS (ESI): m/z 409.0 [M+H]⁺.

### Example 364

### (3S,6S)-4-(3-(4-fluorophenyl)-1,2,4-oxadiazole-5-carbonyl)-3-isopropyl-6-phenyl-piperazin-2-one(404)

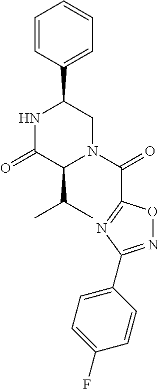

According to the method described for compound 89, compound 401 (52.4 mg, 0.24 mmol) and 3-(4-fluorophenyl)-1,2,4-oxadiazole-5-carboxylic acid (50 mg, 0.24 mmol) were coupled to give the product 404 (12.5 mg, 13% yield) as white solids.

¹H NMR (400 MHz, CDCl3): δ 8.14-8.10 (2H, m), 7.45-7.34 (5H, m), 7.24-7.19 (2H, m), 6.09 (1H, s), 5.15-5.11 (1H, m), 4.97-4.88 (1H, m), 4.65-4.60 (1H, m), 3.52-3.45 (1H, m), 2.61-2.46 (1H, m), 1.29-1.12 (6H, m).

MS (ESI): m/z 408.9 [M+1]⁺.

### Example 365

### (3S,6S)-4-(5-(4-chlorophenyl)-1,2,4-oxadiazole-3-carbonyl)-3,6-diisobutylpiperazin-2-one(405)

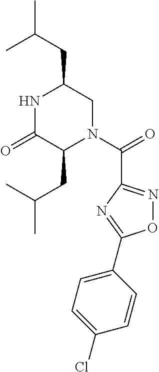

According to the method described for compound 89, compound 7 (94.3 mg, 0.44 mmol) and 5-(4-chlorophenyl)-1,2,4-oxadiazole-3-carboxylic acid (100 mg, 0.44 mmol) were coupled to give the product 405 (39.9 mg, 22% yield) as a colorless oil.

¹H NMR (400 MHz, CDCl3): δ 8.14 (2H, d, J=8.4 Hz), 7.55 (2H, d, J=8.4 Hz), 6.08 (1H, s), 5.29 (1H, dd, J=4.4, 9 Hz), 4.85-4.81 (1H, m), 4.28 (1H, dd, J=3.6, 14.2 Hz), 3.14 (1H, dd, J=11.2, 14.2 Hz), 1.91-1.63 (4H, m), 1.43-1.32 (2H, m), 1.10-0.74 (12H, m).

MS (ESI): m/z 419.0 [M+H]⁺.

### Example 366

### (3S,6S)-4-(5-(4-chlorophenyl)-1,2,4-oxadiazole-3-carbonyl)-3-isobutyl-6-phenyl-piperazin-2-one(406)

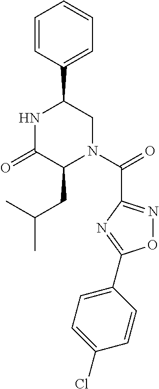

According to the method described for compound 89, compound 11 (51.70 mg, 0.22 mmol) and 5-(4-chlorophenyl)-1,2,4-oxadiazole-3-carboxylic acid (50 mg, 0.22 mmol) were coupled to give the product 406 (19 mg, 20% yield) as a white solid.

¹H NMR (400 MHz, CDCl3): δ 8.17-8.13 (2H, m), 7.56 (2H, d, J=8.4 Hz), 7.43-7.35 (5H, m), 6.03 (1H, s), 5.40 (1H, dd, J=4, 10 Hz), 4.99-4.91 (1H, m), 4.43-4.38 (1H, m), 3.39 (1H, dd, J=11.2, 14.6 Hz), 2.05-1.79 (3H, m), 1.13 (3H, d, J=6 Hz), 1.01 (3H, d, J=6 Hz).

MS (ESI): m/z 439.0 [M+H]⁺.

### Example 367

### (3S,6S)-3-allyl-4-(5-(4-fluorophenyl)isoxazole-3-carbonyl)-6-phenylpiperazin-2-one(407)

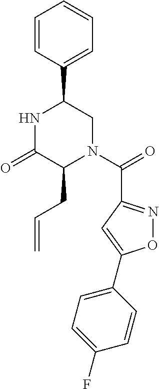

### Step 1: Synthesis of (3S,6S)-3-allyl-6-phenylpiperazin-2-one (408)

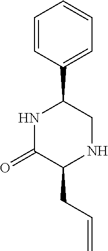

Synthesized from (S)-2-amino-2-phenylacetic acid (30.0 g, 0.19 mol) and (S)-methyl 2-aminopent-4-enoate (6.0 g, 35 mmol) by the method described for the compound 7 (Scheme II) to afford the product 408 (2.0 g, overall yield: 4.6%) as a brown gum.

¹H NMR (400 MHz, DMSO-d₆): δ 7.96 (s, 1H), 7.24-7.36 (m, 5H), 5.81-5.91 (m, 1H), 5.02-5.09 (m, 2H), 4.46 (s, 1H), 3.28-3.31 (m, 1H) 3.08-3.09 (m, 1H), 2.79-2.84 (m, 1H), 2.39-2.44 (m, 1H), 2.24 (s, 1H).

MS (ESI): m/z 217.0 [M+H]⁺

### Step 2

According to the method described for compound 89, compound 408 (50 mg, 0.23 mmol) and 5-(4-fluoro-phenyl)-isoxazole-3-carboxylic acid (57 mg, 0.28 mmol) were coupled to give the product 407 (29.9 mg, yield: 32%) as a colorless oil.

¹H NMR (400 MHz, CDCl3): δ 7.83-7.77 (2H, m), 7.45-7.35 (5H, m), 7.22-7.17 (2H, m), 6.86 (1H, s), 6.16-6.14 (1H, m), 6.00-5.79 (1H, m), 5.36-5.33 (1H, m), 5.18-5.11 (2H, m), 4.95-4.85 (2H, m), 3.39 (1H, dd, J=11.2, 14 Hz), 2.99-2.77 (2H, m).

MS (ESI): m/z 406.0 [M+1]⁺.

### Example 368

### (3S,6S)-3-allyl-4-(5-(4-fluorophenyl)isoxazole-3-carbonyl)-6-propylpiperazin-2-one(409)

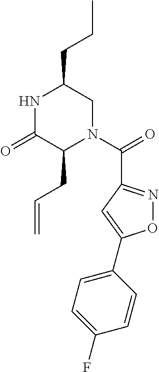

### Step 1: Synthesis of (3S,6S)-3-allyl-6-propylpiperazin-2-one (410)

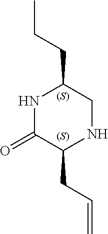

Synthesized from (S)-2-aminopentanoic acid (50.0 g, 427.35 mmol) and (S)-methyl 2-aminopent-4-enoate hydrochloride (9.6 g, 58.01 mmol) by the method described for the compound 7 (Scheme II) to afford the product 410 (4.8 g, overall yield: 6.2%) as an oil.

¹HNMR (400 MHz, CDCl₃) δ 6.68 (s, 1H), 5.73-5.81 (m, 1H), 5.12-5.16 (m, 2H), 3.40-3.43 (m, 1H), 3.33-3.36 (m, 1H), 2.97-3.01 (m, 1H), 2.78-2.81 (m, 1H), 2.60-2.66 (m, 2H), 2.42-2.48 (m, 1H), 1.43-1.50 (m, 2H), 1.30-1.38 (m, 2H), 0.90-0.93 (m, 3H).

MS (ESI): m/z 183.1 [M+H]⁺

### Step 2

According to the method described for compound 89, compound 410 (42 mg, 0.23 mmol) and 5-(4-fluoro-phenyl)-isoxazole-3-carboxylic acid (57 mg, 0.28 mmol) were coupled to give the product 409 (16.5 mg, 19% yield) as a pink solid.

¹H NMR (400 MHz, CDCl3): δ 7.81-7.77 (2H, m), 7.21-7.17 (2H, m), 6.84 (1H, s), 6.08 (1H, m), 5.94-5.72 (1H, m), 5.27-5.23 (1H, m), 5.16-5.07 (2H, m), 4.88-4.79 (1H, m), 3.70-3.58 (1H, m), 3.16 (1H, dd, J=11.2, 14 Hz), 2.92-2.67 (2H, m), 1.58-1.34 (4H, m), 1.01-0.96 (3H, m).

MS (ESI): m/z 371.9 [M+H]⁺.

### Example 369

### (3S,7S)-4-(5-(4-fluorophenyl)isoxazole-3-carbonyl)-3,7-diisobutyl-1,4-diazepan-2-one (411)

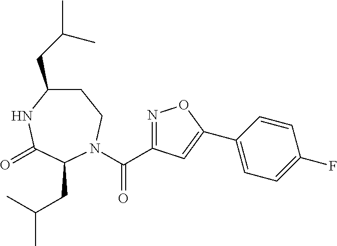

### Step 1: Synthesis of (3S,7S)-3,7-Diisobutyl-[1,4]diazepan-5-one (412)

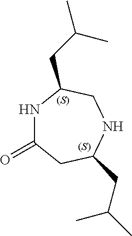

Synthesized from 2-Amino-4-methyl-pentanoic acid (2.61 g, 2.00 mmol) and Hydrochloride salt of 3-Amino-5-methyl-hexanoic acid methyl ester (3.49 g, 1.79 mmol) by the method described for the compound 7 (Scheme II) to afford the product (3S,7S)-3,7-Diisobutyl-[1,4]-diazepan-5-one 412 (220 mg, overall yield: 6.84%).

¹H NMR (400 MHz, CDCl3): δ 5.65 (s, 1H), 3.42-3.46 (m, 1H), 3.03-3.06 (m, 1H), 2.81-2.85 (m, 2H), 2.43-2.48 (m, 1H), 2.12 (s, 1H), 1.61-1.67 (m, 2H), 1.41-1.49 (m, 2H), 1.24-1.35 (m, 2H), 0.85-0.97 (m, 12H).

MS (ESI): 227.2 (M+H)⁺

### Step 2

Compound 412 (23 mg, 0.10 mmol) and 5-(4-fluoro-phenyl)isoxazole-3-carboxylic acid (25 mg, 0.12 mmol) were coupled according to the procedure described for compound 71 to furnish 411 (30 mg, 71.1% yield) as a white solid.

¹H NMR (400 MHz, CDCl₃): δ 7.80-7.77 (m, 2H), 7.20-7.16 (m, 2H), 6.78 (d, J=22.4 Hz, 1H), 5.70 (brs, 1H), 5.25-5.22 (m, 0.5H), 4.80-4.75 (m, 0.5H), 4.69 (d, J=14.4 Hz, 0.5H), 4.37 (d, J=14.4 Hz, 0.5H), 3.70-3.50 (m, 2H), 3.19-2.59 (m, 2H), 1.82-1.35 (m, 6H) and 0.99-0.77 (m, 12H).

MS (ESI): m/z 416.2 (M+H)⁺

### Example 370

### (3S)-4-(5-(4-fluorophenyl)isoxazole-3-carbonyl)-3,6-diisobutyl-1,4-diazepan-2-one (413)

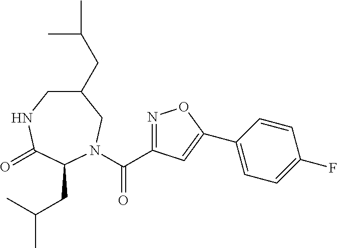

### Step 1: (3S)-3,6-Diisobutyl-[1,4]diazepan-2-one (414)

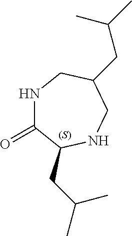

Synthesized from 2-Aminomethyl-4-methyl-pentanoic acid ethyl ester (80.0 g, 0.461 mol) and Hydrochloride salt of 2-Amino-4-methyl-pentanoic acid methyl ester (78.2 g, 0.431 mol) by the method described for the compound 7 (Scheme II) to afford the product 414 (4.8 g, overall yield: 3.42%).

¹H NMR (400 MHz, CDCl3): δ 5.93 (s, 1H), 3.26-3.30 (m, 2H), 3.10-3.14 (m, 2H), 2.50 (dd, J₁=10.8 Hz, J₂=14.0 Hz, 1H), 1.55-1.75 (m, 5H), 1.39-1.43 (m, 1H), 0.86-0.99 (m, 14H)

MS (ESI): 227.1 (M+H)⁺

### Step 2

Compound 414 (46 mg, 0.20 mmol) and 5-(4-fluoro-phenyl)isoxazole-3-carboxylic acid (45 mg, 0.22 mmol) were coupled according to the procedure described for compound 71 to furnish 413 (30 mg, 35.5% yield) as a white solid.

¹H NMR (400 MHz, CDCl₃): δ 7.80-7.76 (m, 2H), 7.20-7.14 (m, 2H), 6.75 (d, J=36.4 Hz, 1H), 6.91 and 5.91 (2brs, 1H), 5.08 (dd, J=10.4 & 3.2 Hz, 0.5H), 4.66-4.61 (m, 1H), 4.47 (d, J=14.4 Hz, 0.5H), 3.31-3.05 (m, 4H), 2.30-1.20 (m, 6H) and 1.01-0.79 (m, 12H).

MS (ESI): m/z 416.2 (M+H)⁺

### Example 371

### (3S,7S)-4-(5-(4-fluorophenyl)isoxazole-3-carbonyl)-3-isobutyl-7-phenyl-1,4-diazepan-2-one (415)

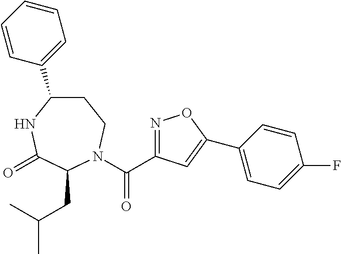

### Step 1: Synthesis of (3S,7S)-3-Isobutyl-7-phenyl-[1,4]diazepan-2-one (416)

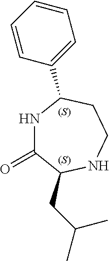

Synthesized from 3-Amino-3-phenyl-propionic acid (12.5 g, 75.76 mmol) and Hydrochloride salt of 2-Amino-4-methyl-pentanoic acid methyl ester (18.13 g, 10.02 mmol) by the method described for the compound 7 (Scheme II) to afford the product 416 (475 mg, overall yield: 3.84%).

¹H NMR (400 MHz, CDCl3): δ 7.35-7.45 (m, 1H), 5.60 (s, 1H), 4.65 (dd, J₁=4.0 Hz, J₂=10.4 Hz, 1H), 3.37-3.46 (m, 2H), 3.03-3.09 (m, 1H), 1.98-2.05 (m, 1H), 1.72-1.88 (m, 3H), 1.42-1.48 (m, 1H), 0.92-0.98 (m, 6H).

MS (ESI): 247.0 (M+H)⁺

### Step 2

Compound 416 (50 mg, 0.20 mmol) and 5-(4-fluoro-phenyl)isoxazole-3-carboxylic acid (45 mg, 0.22 mmol) were coupled according to the procedure described for compound 71 to furnish 415 (20 mg, 21.5% yield) as a white solid.

¹H NMR (400 MHz, CDCl₃): δ 7.82-7.77 (m, 2H), 7.39-7.31 (m, 5H), 7.21-7.17 (m, 2H), 6.89 (s, 1H), 5.91 (d, J=19.2 Hz, 1H), 5.61 (brs, 0.5H), 5.40 (dd, J=9.8 & 3.2 Hz, 0.5H), 4.76-4.54 (m, 2H), 3.58 (brs, 0.5H), 3.25 (m, 0.5H), 2.48-1.95 (m, 3H), 1.90-1.56 (m, 2H) and 1.05-0.77 (m, 6H).

MS (ESI): m/z 458.1 (M+Na)⁺

### Example 372

### (3S,75)-3-isobutyl-7-phenyl-4-((1R,2R)-2-phenylcyclopropanecarbonyl)-1,4-diazepan-2-one (417)

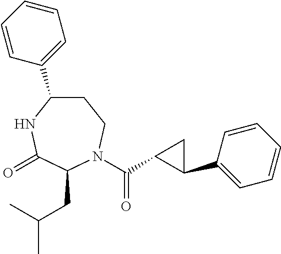

Compound 416 (50 mg, 0.20 mmol) and (1R,2R)-2-phenylcyclopropanecarboxylic acid 54 (35 mg, 0.22 mmol) were coupled according to the procedure described for compound 71 to furnish 417 (55 mg, 69.4% yield) as a white solid.

¹H NMR (400 MHz, CDCl₃): δ 7.42-7.10 (m, 10H), 6.89 (s, 1H), 5.96 (brs, 1H), 5.51 (brs, 0.5H), 4.95 (brs, 0.5H), 4.66-4.45 (m, 1.5H), 4.04 (brs, 0.5H), 3.53 (brs, 0.5H), 3.03 (brs, 0.5H), 2.70-2.05 (m, 5H), 1.90-1.59 (m, 3H), 1.38-1.33 (m, 1H) and 1.00-0.96 (m, 6H).

MS (ESI): m/z 391.1 (M+H)⁺

### Example 373

### (3S,6S)-3-isobutyl-6-phenyl-4-((1R,2R)-2-phenylcyclopropanecarbonyl)-1,4-diazepan-2-one (418)

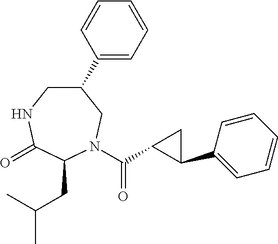

### Step 1: Synthesis of (3S,6R)-3-Isobutyl-6-phenyl-[1,4]diazepan-2-one (419)

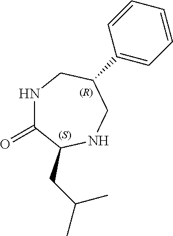

Synthesized from Hydrochloride salt of 3-Amino-2-phenyl-propionic acid (5.0 g, 24.80 mmol) and Hydrochloride salt of 2-Amino-4-methyl-pentanoic acid methyl ester (6.4 g, 35.53 mmol) by the method described for the compound 7 (Scheme II) to afford the product 419 (560 mg, overall yield: 9.28%).

¹H NMR (400 MHz, CDCl3): δ 7.31-7.33 (m, 2H), 7.22-7.28 (m, 1H), 7.13-7.15 (m, 2H), 6.09 (s, 1H), 3.70-3.77 (m, 1H), 3.41-3.47 (m, 2H), 3.28-3.34 (m, 1H), 3.03 (dd, J₁=11.2 Hz, J₂=13.6 Hz, 1H), 2.76-2.82 (m, 1H), 1.70-1.85 (m, 2H), 1.41-1.55 (m, 2H), 0.92-0.98 (m, 6H).

MS (ESI): 247.1 (M+H)⁺

### Step 2

Compound 419 (50 mg, 0.20 mmol) and (1R,2R)-2-phenylcyclopropanecarboxylic acid 54 (35 mg, 0.22 mmol) were coupled according to the procedure described for compound 71 to furnish 418 (30 mg, 37.8% yield) as a white solid.

¹H NMR (400 MHz, CDCl₃): δ 7.36-6.98 (m, 10H), 6.35 (brs, 1H), 5.51 (brs, 0.5H), 4.04 (brs, 0.5H), 3.77-3.60 (m, 1.5H), 3.31-3.10 (m, 3H), 2.60-2.35 (m, 1H), 2.15-1.59 (m, 6H), 1.38-1.05 (m, 1H) and 0.97-0.91 (m, 6H).

MS (ESI): m/z 391.1 (M+H)⁺

### Example 374

### (3S,6S)-3-isobutyl-6-phenyl-4-(5-(4-fluorophenyl)isoxazole-3-carbonyl)-1,4-diazepan-2-one (420)

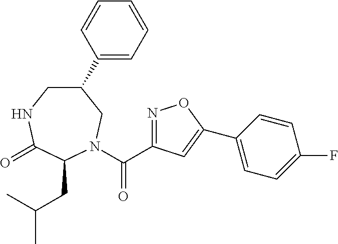

Compound 419 (50 mg, 0.20 mmol) and 5-(4-fluoro-phenyl)isoxazole-3-carboxylic acid (45 mg, 0.22 mmol) were coupled according to the procedure described for compound 71 to furnish 420 (12 mg, 13.6% yield) as a white solid.

¹H NMR (400 MHz, CDCl₃): δ 7.81-7.75 (m, 2H), 7.40-7.15 (m, 8H), 6.70 (s, 1H), 6.19 (d, J=8.0 Hz, 1H), 6.07 (brs, 0.5H), 5.16 (dd, J=11.2 & 4.0 Hz, 0.5H), 4.90-4.85 (m, 0.5H), 3.90-3.83 (m, 0.5H), 3.76-3.70 (m, 0.5H), 3.45-3.38 (m, 0.5H), 3.32-3.26 (m, 1H), 3.11 (dd, J=13.6 & 8.0 Hz, 0.5H), 2.05-1.90 (m, 2H), 1.72-1.62 (m, 1H), 1.58 (s, 2H) and 1.03-0.85 (m, 6H).

MS (ESI): m/z 436.1 (M+Na⁺

### Example 375

### (3S,6S)-4-[(1S,2S)-2-(2,4-Difluoro-phenyl)-cyclopropanecarbonyl]-3,6-diisobutyl-piperazin-2-one (421)

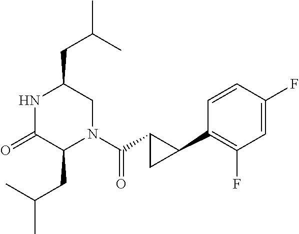

Compound 7 (42 mg, 0.20 mmol) and trans (S,S)[2-(2,4-di-fluoro)phenyl]-cyclopropyl-1-carboxylic acid 64 (40 mg, 0.20 mmol) were coupled according to the method described for the preparation of compound 71 to furnish product 421 (35 mg, 42.8% yield) as a white solid.

¹H NMR (400 MHz, CDCl₃): δ 7.05-6.73 (m, 3H), 6.10 (brs, 1H), 5.21 (dd J=9.6 & 4.0 Hz, 0.5H), 4.71 (dd, J=13.6 & 4.0 Hz, 0.5H), 4.57 (dd, J=9.6 & 4.0 Hz, 0.5H), 4.01 (dd, J=13.6 & 4.0 Hz, 0.5H), 3.60-3.51 (m, 1H), 3.09 (dd, J=14.0 & 11.2 Hz, 0.5H), 2.67-2.59 (m, 1H), 2.45 (dd, J=14.0 & 11.2 Hz, 0.5H), 1.96-1.86 (m, 1H), 1.82-1.56 (m, 5H), 1.42-1.20 (m, 3H) and 1.01-0.67 (m, 12H).

MS (ESI): m/z 393.2 [M+H]⁺

### Example 376

### (3S,6S)-3-isobutyl-4-((1R,2R)-2-phenylcyclopropanecarbonyl)-6-((E)-prop-1-en-1-yl)piperazin-2-one (422)

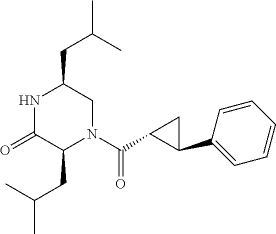

### Step 1: Synthesis of (3S,6S)-3-Isobutyl-6-propenyl-piperazin-2-one (423)

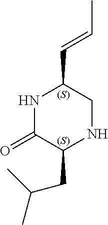

Synthesized from 2-Amino-pent-3-enoic acid (11.93 g, 104 mmol) and Hydrochloride salt of L-Leucine methyl ester (7.8 g, 42.95 mmol) by the method described for the compound 7 (Scheme II) to afford the product 423 (1.623 g, overall yield: 7.9%) as a white solid.

¹H NMR (400 MHz, DMSO-d6): δ 7.56 (s, 1H), 5.43-5.57 (m, 2H), 3.74 (s, 1H), 3.07 (dd, J₁=10.8 Hz, J₂=3.2 Hz, 1H), 2.82 (dd, J₁=12.8 Hz, J₂=4.8 Hz, 1H), 2.63 (dd, J₁=12.8 Hz, J₂=5.6 Hz, 1H), 1.74-1.82 (m, 1H), 1.64 (d, J=7.2 Hz, 3H), 1.47-1.54 (m, 1H), 1.35-1.42 (m, 1H), 0.82-0.88 (m, 7H).

MS (ESI): m/z 197.1635 [M+H]⁺

### Step 2

Compound 423 (40 mg, 0.20 mmol) and trans (R,R)(2-phenyl)-cyclopropyl-1-carboxylic acid 54 (35 mg, 0.22 mmol) were coupled according to the procedure described for compound 71 to furnish 422 (12 mg, 17.3% yield) as a white solid.

¹H NMR (400 MHz, CDCl₃): δ 7.31-7.06 (m, 5H), 5.86-5.76 (m, 2H), 5.33-5.27 (m, 1H), 5.19 (dd, J=9.6 & 4.0 Hz, 0.5H), 4.65 (dd, J=13.6 & 4.0 Hz, 0.5H), 4.55 (dd, J=9.6 & 4.0 Hz, 0.5H), 4.06-3.96 (m, 1.5H), 3.17 (dd, J=14.4 & 11.2 Hz, 0.5H), 2.70 (dd, J=14.4 & 11.2 Hz, 0.5H), 2.56-2.51 (m, 1H), 1.98-1.60 (m, 8H), 1.36-1.28 (m, 1H) and 1.02-0.93 (m, 6H).

MS (ESI): m/z 341.1 [M+H]⁺

### Example 377

### (3S,6S)-4-(5-(4-fluorophenyl)isoxazole-3-carbonyl)-3-isobutyl-6-((E)-prop-1-en-1-yl)piperazin-2-one (424)

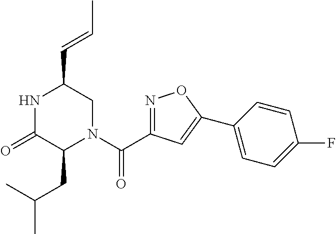

Compound 423 (40 mg, 0.20 mmol) and 5-(4-fluorophenyl)isoxazole-3-carboxylic acid (45 mg, 0.22 mmol) were coupled according to the procedure described for compound 71 to furnish 424 (32 mg, 40.7% yield) as a white solid.

¹H NMR (400 MHz, CDCl₃): δ 7.80-7.76 (m, 2H), 7.20-7.15 (m, 2H), 6.86 (s, 1H), 5.92-5.81 (m, 2H), 5.34-5.25 (m, 2H), 4.80-4.72 (m, 1H), 4.20-4.13 (m, 1H), 3.19 (dd, J=14 & 11.2 Hz, 1H), 1.92-1.68 (m, 6H) and 1.08-0.77 (m, 6H).

MS (ESI): m/z 386.2 [M+H]⁺

### Example 378

### (3S,6S)-4-((5-(4-fluorophenyl)isoxazol-3-yl)methyl)-3-isobutyl-6-((E)-prop-1-en-1-yl)piperazin-2-one (425)

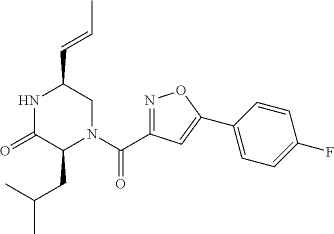

Compound 423 (50 mg, 0.25 mmol) and 5-(4-fluorophenyl)isoxazole-3-carbaldehyde (50 mg, 0.25 mmol) were coupled according to the procedure described for compound 195 to furnish 425 (37 mg, 39% yield) as a colorless solid.

¹H NMR (400 MHz, CDCl₃): δ 7.78-7.73 (m, 2H), 7.18-7.13 (m, 2H), 6.48 (s, 1H), 5.72 (s, 1H), 5.70-5.65 (m, 1H), 5.34-5.27 (m, 1H), 4.23-4.17 (m, 1H), 3.99-3.82 (m, 2H), 3.24-3.21 (m, 1H), 2.85-2.68 (m, 2H), 1.91-1.84 (m, 1H), 1.76-1.56 (m, 5H), 0.96-0.86 (m, 6H).

MS (ESI): m/z 372.2 [M+H]⁺

### Example 379

### (3S,6R)-4-(5-(4-fluorophenyl)isoxazole-3-carbonyl)-3-isobutyl-6-(thiophen-2-yl)piperazin-2-one (426)

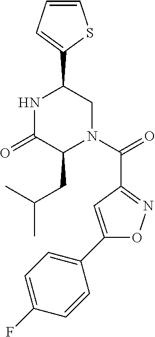

### Step 1: Synthesis of (3S,6R)-3-Isobutyl-6-thiophen-2-yl-piperazin-2-one (427)

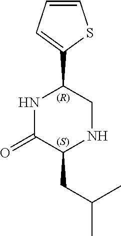

Synthesized from (R)-Amino-thiophen-2-yl-acetic acid (5.0 g, 31.85 mmol) and Hydrochloride salt of L-Leucine methyl ester (38.6 g, 47.78 mmol) by the method described for the compound 7 (Scheme II) to afford the product 427 (610 mg, overall yield: 8.0%) as a white solid.

¹H NMR (400 MHz, CDCl3): δ 7.26-7.28 (m, 1H), 6.97-7.01 (m, 2H), 5.96 (s, 1H), 4.88 (dd, J₁=4.4 Hz, J₂=9.2 Hz, 1H), 3.49 (dd, J₁=3.6 Hz, J₂=10.0 Hz, 1H), 3.36-3.41 (m, 1H), 2.93-2.99 (m, 1H), 1.88-1.94 (m, 1H), 1.75-1.84 (m, 1H), 1.63-1.71 (m, 1H), 1.54-1.60 (m, 1H), 0.94-0.99 (m, 6H).

MS (ESI): m/z 239.1 [M+H]⁺

### Step 2

According to the method described for compound 89, compound 427 (112 mg, 0.46 mmol) and 5-(4-Fluoro-phenyl)-isoxazole-3-carboxylic acid (117.2 mg, 0.57 mmol) were coupled to give the product 426 (100.1 mg, 50% yield) as an yellow solid.

¹H NMR (400 MHz, CDCl3): δ 7.81-7.77 (2H, m), 7.35-7.32 (1H, m), 7.21-7.12 (3H, m), 7.04-7.00 (1H, m), 6.89 (1H, s), 6.45 (1H, s), 5.35-5.31 (1H, m), 5.18-5.15 (1H, m), 5.04-4.99 (1H, m), 3.51-3.45 (1H, m), 1.95-1.71 (3H, m), 1.09 (3H, d, J=6.4 Hz), 1.00 (3H, d, J=6.4 Hz).

MS (ESI): m/z 428.0 [M+H]⁺.

### Example 380

### (3S,6S)-4-(5-(4-fluorophenyl)isoxazole-3-carbonyl)-3-isobutyl-6-(thiophen-2-yl)piperazin-2-one (428)

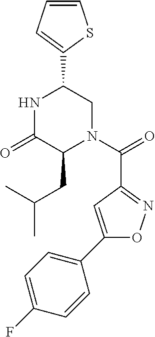

### Step 1: Synthesis of (3S,6S)-3-Isobutyl-6-thiophen-2-yl-piperazin-2-one (429)

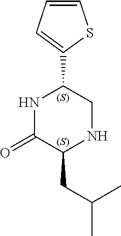

Synthesized from (S)-Amino-thiophen-2-yl-acetic acid (2.0 g, 12.74 mmol) and Hydrochloride salt of L-Leucine methyl ester (2.8 g, 15.62 mmol) by the method described for the compound 7 (Scheme II) to afford the product 429 (562 mg, overall yield: 18.4%) as a white solid.

¹H NMR (400 MHz, CDCl3): δ 7.25-7.27 (m, 1H), 6.96-7.00 (m, 2H), 6.50 (s, 1H), 4.86 (dd, J₁=3.6 Hz, J₂=7.2 Hz, 1H), 3.46-3.49 (m, 1H), 3.28 (dd, J₁=4.0 Hz, J₂=13.2 Hz, 1H), 3.10 (dd, J₁=4.0 Hz, J₂=13.2 Hz, 1H), 1.76-1.88 (m, 3H), 1.56-1.60 (m, 1H), 0.94 (dd, J₁=6.0 Hz, J₂=12.0 Hz, 6H).

MS (ESI): m/z 239.1 [M+H]⁺

### Step 2

According to the method described for compound 89, compound 429 (112 mg, 0.46 mmol) and 5-(4-Fluoro-phenyl)-isoxazole-3-carboxylic acid (117.2 mg, 0.57 mmol) were coupled to give the product 428 as a light brown solid. (62.5 mg, 31% yield)

¹H NMR (400 MHz, CDCl3): δ 7.68-7.64 (2H, m), 7.18-7.14 (3H, m), 6.89-6.87 (1H, m), 6.84 (1H, s), 6.75 (1H, s), 6.11 (1H, s), 5.48 (1H, dd, J=4.4, 9.8 Hz), 4.91-4.80 (2H, m), 4.01 (1H, dd, J=3.6, 14.4 Hz), 1.95-1.71 (3H, m), 1.05 (3H, d, J=6.4 Hz), 0.99 (3H, d, J=6.4 Hz).

MS (ESI): m/z 428.0 [M+H]⁺.

### Example 381

### (3S,6R)-4-(5-(4-fluorophenyl)-1,2,4-oxadiazole-3-carbonyl)-3-isobutyl-6-(thiophen-2-yl)piperazin-2-one(430)

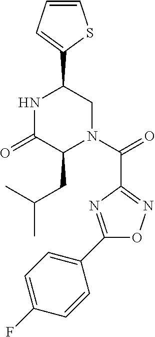

According to the method described for compound 89, compound 427 (57.2 mg, 0.24 mmol) and 66 (50 mg, 0.24 mmol) were coupled to give the product 430 (11.2 mg, 11% yield) as a colorless oil.

¹H NMR (400 MHz, CDCl3): δ 8.24-8.20 (2H, m), 7.34-7.33 (1H, m), 7.28-7.23 (2H, m), 7.11-7.10 (1H, m), 7.03-7.00 (1H, m), 6.04 (1H, s), 5.39-5.36 (1H, m), 5.22 (1H, dd, J=4, 11 Hz), 4.50-4.45 (1H, m), 3.52 (1H, dd, J=10.8, 14.4 Hz), 2.01-1.77 (3H, m), 1.12 (3H, d, J=6.4 Hz), 1.02 (3H, d, J=6.4 Hz).

MS (ESI): m/z 429.1 [M+1]⁺.

### Example 382

### (3S,6R)-4-(3-(4-fluorophenyl)-1,2,4-oxadiazole-5-carbonyl)-3-isobutyl-6-(thiophen-2-yl)piperazin-2-one(431)

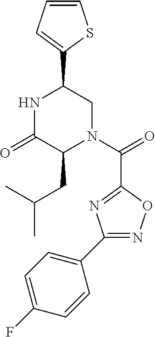

According to the method described for compound 89, compound 427 (57.2 mg, 0.24 mmol) and 3-(4-fluorophenyl)-1,2,4-oxadiazole-5-carboxylic acid (50 mg, 0.24 mmol) were coupled to give the product 431 (70.2 mg, 68% yield) as a colorless oil.

¹H NMR (400 MHz, CDCl3): δ 8.13-8.09 (2H, m), 7.37-7.35 (1H, m), 7.24-7.18 (2H, m), 7.15-7.12 (1H, m), 7.06-7.03 (1H, m), 6.36 (1H, s), 5.37-5.30 (1H, m), 5.25 (1H, dd, J=4, 11 Hz), 4.84-4.80 (1H, m), 3.54 (1H, dd, J=10.8, 14.4 Hz), 2.02-1.69 (3H, m), 1.10 (3H, d, J=6.8 Hz), 1.01 (3H, d, J=6.8 Hz).

MS (ESI): m/z 429.1 [M+H]⁺.

### Example 383

### (3S,6R)-4-(3-(4-fluorophenyl)isoxazole-5-carbonyl)-3-isobutyl-6-(thiophen-2-yl)piperazin-2-one(432)

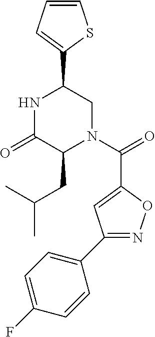

According to the method described for compound 89, compound 427 (50 mg, 0.21 mmol) and 3-(4-fluorophenyl)-isoxazole-5-carboxylic acid (52.2 mg, 0.25 mmol) were coupled to give the product 432 (45.3 mg, yield: 50% yield) as a white solid.

¹H NMR (400 MHz, CDCl3): δ 7.83-7.79 (2H, m), 7.35-7.33 (1H, m), 7.21-7.12 (4H, m), 7.05-7.01 (1H, m), 6.45 (1H, s), 5.30-5.26 (1H, m), 5.14 (1H, dd, J=4, 10.8 Hz), 4.61 (1H, dd, J=4, 10.2 Hz), 3.56 (1H, dd, J=10.8, 14.4 Hz), 2.03-1.70 (3H, m), 1.08 (3H, d, J=6.4 Hz), 1.01 (3H, d, J=6.4 Hz).

MS (ESI): m/z 428.1 [M+H]⁺.

### Example 384

### (3S,6R)-4-((1R,2R)-2-(4-fluorophenyl)cyclopropanecarbonyl)-3-isobutyl-6-(thiophen-2-yl)piperazin-2-one(433)

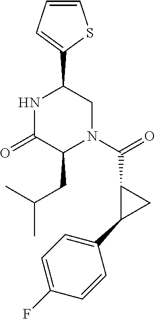

According to the method described for compound 89, compound 427 (50 mg, 0.21 mmol) and (1R,2R)-2-(4-Fluoro-phenyl)-cyclopropanecarboxylic acid 60 (45.4 mg, 0.25 mmol) were coupled to give the product 433 (56.6 mg, yield: 67% yield) as a colorless oil.

¹H NMR (400 MHz, CDCl3): δ 7.33-7.30 (1H, m), 7.09-6.94 (6H, m), 6.22 (1H, s), 4.99-4.93 (1H, m), 4.88-4.83 (1H, m), 4.63-4.59 (1H, m), 4.26-4.22 (1H, m), 2.99 (1H, dd, J=10.8, 14.4 Hz), 2.59-2.52 (1H, m), 1.95-1.56 (5H, m), 1.04-0.94 (6H, m).

MS (ESI): m/z 401.1 [M+H]⁺.

### Example 385

### (3S,6R)-4-((E)-3-(2,4-difluorophenyl)acryloyl)-3-isobutyl-6-(thiophen-2-yl)pipera-zin-2-one(434)

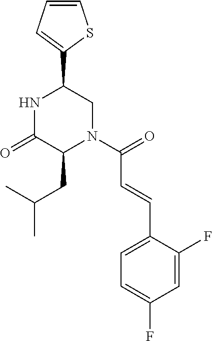

According to the method described for compound 89, compound 427 (50 mg, 0.21 mmol) and (E)-3-(2,4-Difluoro-phenyl)-acrylic acid (46.4 mg, 0.25 mmol) were coupled to give the product 434 (70.6 mg, yield: 83% yield) as a colorless oil.

¹H NMR (400 MHz, CDCl3): δ 7.75 (1H, J=15.6 Hz), 7.50-7.44 (1H, m), 7.37-7.32 (1H, m), 7.10-6.84 (5H, m), 6.30 (1H, s), 5.02-4.92 (2H, m), 4.63-4.58 (1H, m), 3.09-3.02 (1H, m), 1.96-1.72 (3H, m), 1.07-0.94 (6H, m).

MS (ESI): m/z 405.1 [M+H]⁺

### Example 386

### (3S,6R)-4-((5-(4-fluorophenyl)isoxazol-3-yl)methyl)-3-isobutyl-6-(thiophen-2-yl)piperazin-2-one(435)

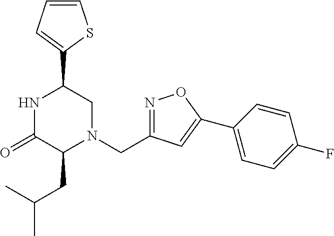

According to the method described for compound 195, compound 427 (50 mg, 0.21 mmol) and 5-(4-Fluoro-phenyl)-isoxazole-3-carbaldehyde (40.14 mg, 0.21 mmol) were coupled to give the product 435 (24.2 mg, 28% yield) as colorless oil.

¹H NMR (400 MHz, CDCl3): δ 7.75-7.72 (2H, m), 7.27-7.26 (1H, m), 7.18-7.13 (2H, m), 6.98-6.95 (2H, m), 6.40 (1H, s), 6.03 (1H, s), 5.15-5.11 (1H, m), 4.01 (1H, d, J=14 Hz), 3.93 (1H, d, J=14 Hz), 3.32 (1H, dd, J=5.2, 8 Hz), 3.11 (1H, dd, J=9, 13.8 Hz), 2.95 (1H, dd, J=4.8, 14 Hz), 1.96-1.80 (3H, m), 1.75-1.68 (1H, m), 0.98 (3H, d, J=6.4 Hz), 0.91 (3H, d, J=6.4 Hz).

MS (ESI): m/z 414.1 [M+H]⁺.

### Example 387

### (3S,6S)-4-(5-(4-fluorophenyl)isoxazole-3-carbonyl)-3-isobutyl-6-(thiophen-3-yl)piperazin-2-one (436)

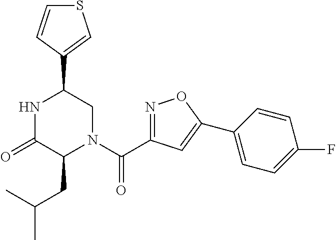

### Step 1: Synthesis of (3S,6S)-3-Isobutyl-6-thiophen-3-yl-piperazin-2-one (437)

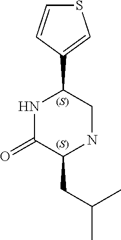

Synthesized from Amino-thiophen-3-yl-acetic acid (4 g, 25 mmol) and Hydrochloride salt of L-Leucine methyl ester (3.728 g, 20.6 mmol) by the method described for the compound 7 (Scheme 1) to afford the product 437 (1.1 g, overall yield: 18.4%) as a white solid.

¹H NMR (400 MHz, CDCl₃) δ 7.35-7.37 (m, 1H), 7.17-7.18 (m, 1H), 6.99-7.00 (m, 1H), 6.12 (s, 1H), 4.71-4.74 (m, 1H), 3.48-3.51 (m, 1H), 3.23-3.27 (m, 1H), 3.03-3.07 (m, 1H), 1.77-1.90 (m, 3H), 1.52-1.59 (m, 1H), 0.94-0.99 (m, 6H)

MS (ESI): m/z 239.1 [M+H]⁺

### Step 2

Compound 437 (48 mg, 0.20 mmol) and 5-(4-fluorophenyl)-isoxazole-3-carboxylic acid (45 mg, 0.22 mmol) were coupled according to the method described for the compound 71 to furnish product 436 (45 mg, 52.3% yield) as a white solid.

¹H NMR (400 MHz, CDCl₃): δ 7.82-7.77 (m, 2H), 7.42-7.10 (m, 5H), 6.89 (brs, 1H), 6.04 (brs, 1H), 5.44-5.34 (m, 1H), 5.02-4.89 (m, 2H), 3.41 (dd, J=15.2 & 12.0 Hz, 0.5H), 3.13 (dd, J=15.2 & 12.0 Hz, 0.5H), 2.02-1.72 (m, 3H) and 1.11-0.80 (m, 6H).

MS (ESI): m/z 428.0 [M+H]⁺

### Example 388

### (3S,6R)-4-((1R,2R)-2-(4-fluorophenyl)cyclopropanecarbonyl)-3-isobutyl-6-(3-methylthiophen-2-yl)piperazin-2-one (438)

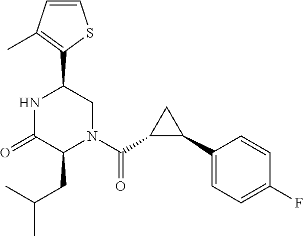

### Step 1: Synthesis of (3S,6R)-3-Isobutyl-6-(3-methyl-thiophen-2-yl)-piperazin-2-one (439) and (3S,6S)-3-Isobutyl-6-(3-methyl-thiophen-2-yl)-piperazin-2-one (440)

Synthesized from Amino-(3-methyl-thiophen-2-yl)-acetic acid (9 g, 52.6 mmol) and Hydrochloride salt of L-Leucine methyl ester (3.6 g, 19.6 mmol) by the method described for the compound 7 (Scheme II) to afford the product 439 (154.33 mg, overall yield 1.15%) and 440 (155.81 mg, overall yield: 1.17%) as white solids.

### Compound 439

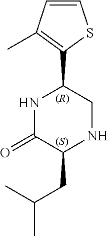

¹H NMR (400 MHz, CDCl₃) δ 7.17-7.18 (m, 1H), 6.81-6.82 (m, 1H), 5.86 (s, 1H), 4.93-4.97 (m, 1H), 3.35-3.52 (m, 1H), 3.30-3.34 (m, 1H), 2.92-2.98 (m, 1H), 2.22 (s, 3H), 1.92-1.98 (m, 1H), 1.68-1.84 (m, 1H), 1.53-1.59 (m, 1H), 0.95-1.01 (m, 6H).

MS (ESI): m/z 253.1 [M+H]⁺

### Compound 440

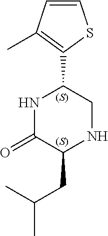

¹H NMR (400 MHz, CDCl₃) δ 7.16-7.17 (m, 1H), 6.83-6.84 (m, 1H), 5.99 (s, 1H), 4.89-4.92 (m, 1H), 3.49-3.52 (m, 1H), 3.22-3.26 (m, 1H), 2.3.04-3.08 (m, 1H), 2.20 (s, 3H), 1.80-1.89 (m, 2H), 1.63-1.68 (m, 1H), 0.94-0.99 (m, 6H).

MS (ESI): m/z 253.1 [M+H]⁺

### Step 2

Compound 439 (40 mg, 0.29 mmol) and 60 (30 mg, 0.17 mmol) were coupled according to the procedure described for compound 71 to furnish compound 438 (49 mg, 79% yield) as colorless solid.

¹H NMR (400 MHz, CDCl₃): δ 7.25-6.86 (m, 6H), 6.82-6.76 (m, 1H), 5.30-5.27 (m, 1H), 4.92-4.90 (m, 1H), 4.73-4.01 (m, 1H), 3.86-3.38 (m, 1H), 2.29-2.24 (m, 1H), 2.13 (s, 3H), 1.88-1.54 (m, 4H), 1.26-1.12 (m, 1H), 1.03-0.70 (m, 6H).

MS (ESI): m/z 415.0 [M+H]⁺

### Example 389

### (3S,6R)-4-(5-(4-fluorophenyl)-1,2,4-oxadiazole-3-carbonyl)-3-isobutyl-6-(3-methylthiophen-2-yl)piperazin-2-one (441)

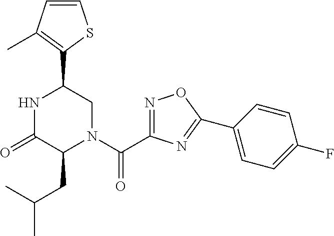

Compound 439 (40 mg, 0.16 mmol) and 66 (42 mg, 0.17 mmol) were coupled according to the procedure described for compound 71 to furnish compound 441 (49 mg, 70% yield) as a colorless solid.

¹H NMR (400 MHz, CDCl₃): δ 8.21-8.07 (m, 2H), 7.26-7.19 (m, 2H), 7.13-7.08 (m, 1H), 6.89-6.79 (m, 1H), 6.64-6.52 (m, 1H), 5.50-5.14 (m, 1H), 5.06-4.91 (m, 1H), 4.80-4.74 (m, 1H), 4.00-3.46 (m, 1H), 1.95 (s, 3H), 1.93-1.71 (m, 3H), 1.16-0.66 (m, 6H).

MS (ESI): m/z 443.0 [M+H]⁺

### Example 390

### (3S,6S)-4-((5-(4-fluorophenyl)isoxazol-3-yl)methyl)-3-isobutyl-6-(thiophen-3-yl)piperazin-2-one (442)

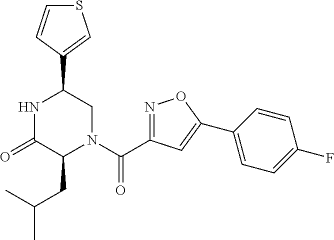

Compound 437 (50 mg, 0.21 mmol) and 5-(4-fluorophenyl)isoxazole-3-carbaldehyde (40 mg, 0.21 mmol) were coupled according to the procedure described for compound 195 to afford 442 (53 mg, 61% yield) as a colorless solid.

¹H NMR (400 MHz, CDCl₃): δ 7.76-7.72 (m, 2H), 7.33-7.31 (m, 1H), 7.20-7.12 (m, 3H), 6.99-6.98 (m, 1H), 6.37 (s, 1H), 6.08 (s, 1H), 5.00-4.96 (m, 1H), 4.03-3.89 (m, 2H), 3.33-3.30 (m, 1H), 3.04-2.99 (m, 1H), 2.91-2.86 (m, 1H), 1.92-1.66 (m, 3H), 0.97-0.89 (m, 6H).

MS (ESI): m/z 414.1 [M+H]⁺

### Example 391

### (3S,6S)-6-cyclobutyl-4-(5-(4-fluorophenyl)isoxazole-3-carbonyl)-3-isobutylpiperazin-2-one (443)

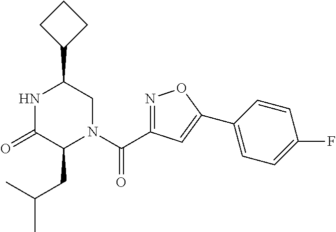

### Step 1: Synthesis of (3S,6S)-6-Cyclobutyl-3-isobutyl-piperazin-2-one (444)

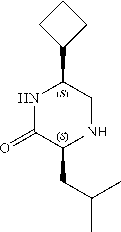

Synthesized from FMOC-(S)-Amino-cyclobutyl-acetic acid (11.3 g, 32.19 mmol) and Hydrochloride salt of L-Leucine methyl ester (3.67 g, 20.3 mmol) by the method described for the compound 7 (Scheme II) to afford the 444 (1.554 g, overall yield: 26.1%) as a white solid.

¹H NMR (400 MHz, CDCl3): δ 5.74 (s, 1H), 3.35-3.38 (m, 1H), 3.24-3.26 (m, 1H), 2.89-2.94 (dd, J₁=4.4 Hz, J₂=13.2 Hz, 1H), 2.68-2.73 (m, 1H), 2.41-2.45 (m, 1H), 1.90-2.04 (m, 3H), 1.63-1.81 (m, 5H), 1.51-1.54 (m, 2H), 0.85-0.94 (m, 6H).

MS (ESI): m/z 211.1 [M+H]⁺

### Step 2

compound 444 (42 mg, 0.20 mmol) and 5-(4-fluorophenyl)isoxazole-3-carboxylic acid (42 mg, 0.20 mmol) were coupled according to the procedure described for compound 71 to furnish 443 (55 mg, 68.9% yield) as a white solid.

¹H NMR (400 MHz, CDCl₃): δ 7.80-7.77 (m, 2H), 7.26-7.16 (m, 2H), 6.87 (s, 1H), 5.91 (s, 1H), 5.30-5.25 (m, 1H), 4.84-5.73 (m, 1H), 3.69-3.55 (m, 1H), 3.09 (dd, J=14.0 & 11.2 Hz, 0.5H), 2.73 (dd, J=14.0 & 11.2 Hz, 0.5H), 2.38-2.29 (m, 1H), 2.14-1.66 (m, 9H), 1.08-0.76 (m, 6H)

MS (ESI): m/z 400.0 [M+H]⁺.

### Example 392

### (3S,6S)-4-((1R,2R)-2-(4-fluorophenyl)cyclopropanecarbonyl)-3-isobutyl-6-(thiophen-3-yl)piperazin-2-one (445)

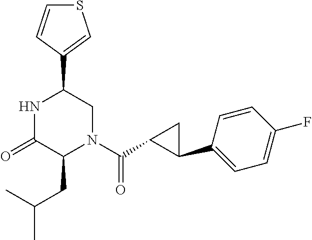

Compound 437 (36 mg, 0.15 mmol) and trans (S,S)[2-(4-fluoro)phenyl]-cyclopropyl-1-carboxylic acid 60 (30 mg, 0.17 mmol) were coupled according to the method described for the preparation of compound 71 to furnish product 445 (20 mg, 33.1% yield) as a white solid.

¹H NMR (400 MHz, CDCl₃): δ 7.39-7.36 (m, 1H), 7.30-7.25 (m, 1H), 7.08-6.93 (m, 5H), 6.30 (brs, 1H), 5.26 (dd, J=8.8 & 4.0 Hz, 0.5H), 4.83-4.75 (m, 1.5H), 4.60 (dd, J=8.8 & 4.0 Hz, 0.5H), 4.17 (dd, J=14.4 & 4.4 Hz, 0.5H), 3.40 (dd, J=14.4 & 11.2 Hz, 0.5H), 2.89 (dd, J=14.4 & 11.2 Hz, 0.5H), 2.57-2.51 (m, 1H), 1.96-1.65 (m, 5H), 1.36-1.24 (m, 1H) and 1.03-0.94 (m, 6H).

MS (ESI): m/z 401.1 [M+H]⁺

### Example 393

### (3S,6S)-4-(5-(4-fluorophenyl)-1,2,4-oxadiazole-3-carbonyl)-3-isobutyl-6-(thiophen-3-yl)piperazin-2-one (446)

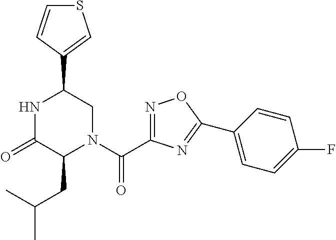

Compound 437 (60 mg, 0.25 mmol) and 5-(4-fluorophenyl)-1,2,4-oxadiazole-3-carboxylic acid 66 (55 mg, 0.26 mmol) were coupled according to the method described for the preparation of compound 71 to furnish product 446 (95 mg, 88.1% yield) as a white solid.

¹H NMR (400 MHz, CDCl₃): δ 8.23-8.18 (m, 2H), 7.42-7.05 (m, 5H), 6.45 (brs, 1H), 5.34 (dd, J=11.2 & 4.4 Hz, 0.5H), 5.03 (dd, J=11.2 & 4.4 Hz, 0.5H), 4.97-4.92 (m, 1.5H), 4.45-4.40 (m, 0.5H), 3.41 (dd, J=14.4 & 11.2 Hz, 0.5H), 3.14 (dd, J=14.4 & 11.2 Hz, 0.5H), 2.00-1.72 (m, 3H) and 1.11-0.73 (m, 6H).

MS (ESI): m/z 429.2 [M+H]⁺

### Example 394

### (3S,6S)-4-(3-(4-fluorophenyl)isoxazole-5-carbonyl)-3-isobutyl-6-((E)-prop-1-en-1-yl)piperazin-2-one (447)

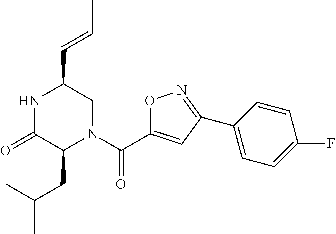

Compound 423 (40 mg, 0.20 mmol) and 3-(4-fluorophenyl)isoxazole-5-carboxylic acid (42 mg, 0.22 mmol) were coupled according to the procedure described for compound 71 to furnish 447 (35 mg, 44.6% yield) as a white solid.

¹H NMR (400 MHz, CDCl₃): δ 7.82-7.79 (m, 2H), 7.19-7.15 (m, 2H), 6.04 (s, 1H), 5.90-5.83 (m, 1H), 5.38-5.28 (m, 1H), 5.23 (dd, J=9.6 & 4.0 Hz, 0.5H), 4.90 (dd, J=9.6 & 4.0 Hz, 0.5H), 4.67 (dd, J=14.0 & 4.0 Hz, 0.5H), 4.40 (dd, J=14.0 & 4.0 Hz, 0.5H), 4.22-4.10 (m, 1H), 3.26 (dd, J=14.4 & 11.2 Hz, 0.5H), 2.94 (dd, J=14.4 & 11.2 Hz, 0.5H), 1.94-1.68 (m, 5H), 1.36-1.26 (m, 1H) and 1.06-0.83 (m, 6H)

MS (ESI): m/z 386.2 [M+H]⁺

### Example 395

### (3S,6S)-4-(5-(4-fluorophenyl)-1,2,4-oxadiazole-3-carbonyl)-3-isobutyl-6-((E)-prop-1-en-1-yl)piperazin-2-one (448)

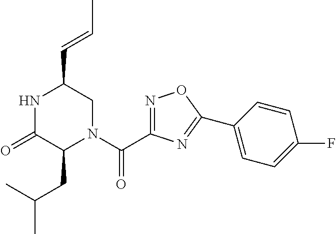

Compound 423 (40 mg, 0.20 mmol) and compound 66 (42 mg, 0.20 mmol) were coupled according to the procedure described for compound 71 to furnish 448 (60 mg, 76.2% yield) as a white solid.

¹H NMR (400 MHz, CDCl₃): δ 8.22-8.17 (m, 2H), 7.26-7.21 (m, 2H), 6.04 (brs, 1H), 5.90-5.81 (m, 1H), 5.38-5.25 (m, 1.5H), 4.85-4.76 (m, 0.5H), 4.23-4.14 (m, 2H), 3.22 (dd, J=15.2 & 12.0 Hz, 0.5H), 2.94 (dd, J=15.2 & 12.0 Hz, 0.5H), 1.92-1.68 (m, 6H) and 1.08-0.71 (m, 6H)

MS (ESI): m/z 387.1 [M+H]⁺.

### Example 396

### (3S,6S)-4-((E)-3-(2,4-difluorophenyl)acryloyl)-3-isobutyl-6-((E)-prop-1-en-1-yl)piperazin-2-one (449)

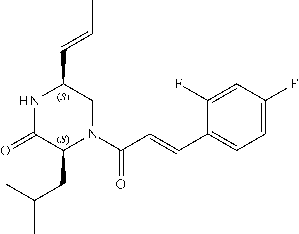

Compound 423 (40 mg, 0.20 mmol) and 2,4-difluoro-cinnamic acid (40 mg, 0.22 mmol) were coupled according to the procedure described for compound 71 to furnish 449 (55 mg, 74.5% yield) as a white solid.

¹H NMR (400 MHz, CDCl₃): δ 7.70 (d, J=15.6 Hz, 1H), 7.47-7.41 (m, 1H), 6.96-6.82 (m, 3H), 5.92 (s, 1H), 5.86-5.79 (m, 1H), 5.35-5.29 (m, 2H), 4.72 (dd, J=13.6 & 4.0 Hz, 0.5H), 4.51 (dd, J=13.6 & 4.0 Hz, 0.5H), 4.07-3.94 (m, 1H), 3.21 (dd, J=15.2 & 12.0 Hz, 0.5H), 2.76 (dd, J=15.2 & 12.0 Hz, 0.5H), 1.88-1.72 (m, 6H) and 1.04-0.94 (m, 6H)

MS (ESI): m/z 363.1 [M+H]⁺

### Example 397

### (3S,6S)-4-(3-(4-fluorophenyl)isoxazole-5-carbonyl)-3-isobutyl-6-(thiophen-3-yl)piperazin-2-one (450)

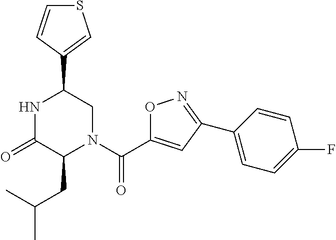

Compound 437 (24 mg, 0.10 mmol) and 3-(4-fluorophenyl)-isoxazole-5-carboxylic acid (23 mg, 0.11 mmol) were coupled according to the method described for the preparation of compound 71 to furnish product 450 (8 mg, 18.6% yield) as a white solid.

¹H NMR (400 MHz, CDCl₃): δ 7.83-7.79 (m, 2H), 7.42-7.40 (m, 1H), 7.35-7.33 (m, 1H), 7.20-7.09 (m, 4H), 6.08 (s, 1H), 5.32-5.29 (m, 0.5H), 5.05-4.83 (m, 2H), 4.58 (dd, J=14.4 & 4.4 Hz, 0.5H), 3.46 (dd, J=14.4 & 11.2 Hz, 0.5H), 3.15 (dd, J=14.4 & 11.2 Hz, 0.5H), 2.02-1.72 (m, 3H) and 1.10-0.88 (m, 6H).

MS (ESI): m/z 428.2 [M+H]⁺

### Example 398

### (3S,6S)-4-((E)-3-(2,4-difluorophenyl)acryloyl)-3-isobutyl-6-(thiophen-3-yl)piperazin-2-one (451)

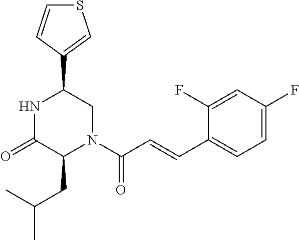

Compound 437 (25 mg, 0.10 mmol) and 2,4-difluoro-cinnamic acid (20 mg, 0.11 mmol) were coupled according to the method described for the preparation of compound 71 to furnish product 451 (15 mg, 35.4% yield) as a white solid.

¹H NMR (400 MHz, CDCl₃): δ 7.76 (d, J=15.2 Hz, 1H), 7.51-7.39 (m, 2H), 7.32 (brs, 1H), 7.15-7.08 (m, 1H), 7.01-6.85 (m, 3H), 6.33 (s, 1H), 5.42 (dd, J=10.0 & 4.0 Hz, 0.5H), 4.94 (dd, J=10.0 & 4.0 Hz, 0.5H), 4.83 (dd, J=9.6 & 4.4 Hz, 0.5H), 4.76 (dd, J=9.6 & 4.4 Hz, 0.5H), 4.62 (dd, J=8.8 & 4.0 Hz, 0.5H), 4.15 (dd, J=8.8 & 4.0 Hz, 0.5H), 3.46 (dd, J=13.6 & 11.2 Hz, 0.5H), 2.97 (dd, J=13.6 & 11.2 Hz, 0.5H), 1.98-1.66 (m, 3H) and 1.08-0.97 (m, 6H).

MS (ESI): m/z 405.2 [M+H]⁺

### Example 399

### (3S,6S)-4-((1R,2R)-2-(4-fluorophenyl)cyclopropanecarbonyl)-3-isobutyl-6-((E)-prop-1-en-1-yl)piperazin-2-one (452)

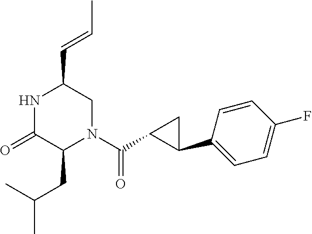

Compound 423 (20 mg, 0.10 mmol) and trans (S,S)[2-(4-fluoro)phenyl]-cyclopropyl-1-carboxylic acid 60 (20 mg, 0.11 mmol) were coupled according to the procedure described for compound 71 to furnish 452 (23 mg, 63% yield) as a white solid.

¹H NMR (400 MHz, CDCl₃): δ 7.11-6.95 (m, 4H), 5.87-5.75 (m, 2H), 5.71 (s, 1H), 5.34-5.28 (m, 1H), 5.21 (dd, J=9.6 & 4.0 Hz, 0.5H), 4.67-4.62 (m, 0.5H), 4.53 (dd, J=9.6 & 4.0 Hz, 0.5H), 4.07-3.98 (m, 1.5H), 3.18 (dd, J=14.0 & 11.2 Hz, 0.5H), 2.70 (dd, J=14.0 & 11.2 Hz, 0.5H), 2.58-2.50 (m, 1H), 1.92-1.62 (m, 9H), 1.32-1.25 (m, 1H) and 1.03-0.94 (m, 6H)

MS (ESI): m/z 359.2 [M+H]⁺.

### Example 400

### (3S,6R)-4-(5-(4-fluorophenyl)isoxazole-3-carbonyl)-3-isobutyl-6-(5-methylthiophen-2-yl)piperazin-2-one (453)

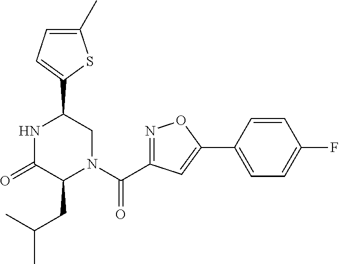

### Step 1: Synthesis of (3S,6R)-3-Isobutyl-6-(5-methyl-thiophen-2-yl)-piperazin-2-one (454) and (3S,6S)-3-Isobutyl-6-(5-methyl-thiophen-2-yl)-piperazin-2-one (455)

Synthesized from Amino-(5-methyl-thiophen-2-yl)-acetic acid (9 g, 52.6 mmol) and Hydrochloride salt of L-Leucine methyl ester (6.37 g, 35.2 mmol) by the method described for the compound 7 (Scheme II) to afford the product 454 (0.57 g, overall yield: 4.2%) and product 455 (0.47 g, overall yield: 3.5%) as a white solid.

### Compound 454

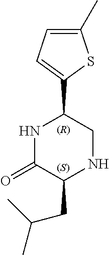

¹H NMR (400 MHz, CDCl₃) δ 7.34-7.35 (m, 1H), 6.77-6.78 (m, 1H), 6.73-6.74 (m, 1H), 6.60-6.61 (s, 1H), 6.02 (s, 1H), 5.79 (s, 2H), 4.76-4.79 (m, 1H), 3.45-3.48 (m, 1H), 3.33-3.37 (m, 1H), 2.90-2.95 (m, 1H), 2.51 (s, 3H), 2.45 (s, 3H), 1.78-1.90 (m, 4H), 1.52-1.58 (m, 1H), 0.94-0.99 (m, 6H).

MS (ESI): m/z 253.15 [M+H]⁺

### Compound 455

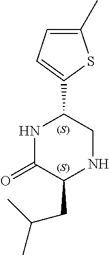

¹H NMR (400 MHz, CDCl₃) δ 6.72-6.73 (m, 1H), 6.60-6.62 (m, 1H), 6.09 (s, 1H), 4.75-4.76 (m, 1H), 3.45-3.48 (m, 1H), 3.24-3.28 (m, 1H), 3.05-3.09 (m, 1H), 2.45 (s, 3H), 1.82-1.86 (m, 2H), 1.54-1.59 (m, 2H), 0.93-0.98 (m, 6H).

MS (ESI): m/z 253.13 [M+H]⁺

### Step 2

Compound 454 (50 mg, 0.20 mmol) and 5-(4-fluorophenyl)-isoxazole-3-carboxylic acid (45 mg, 0.22 mmol) were coupled according to the method described for the preparation of compound 71 to furnish product 453 (40 mg, 45.7% yield) as a white solid.

¹H NMR (400 MHz, CDCl₃): δ 7.81-7.77 (m, 2H), 7.21-7.16 (m, 2H), 6.90-6.87 (m, 2H), 6.66-6.64 (m, 1H), 6.08 (brs, 1H), 5.41-5.29 (m, 1H), 5.06-4.91 (m, 2H), 3.46 (dd, J=14.0 & 9.8 Hz, 0.75H), 3.19 (dd, J=14.0 & 9.8 Hz, 0.25H), 2.47 (s, 3H), 1.98-1.70 (m, 3H) and 1.10-0.80 (m, 6H).

MS (ESI): m/z 442.2 [M+H]⁺

### Example 401

### (3S)-4-(5-(4-fluorophenyl)isoxazole-3-carbonyl)-3-isobutyl-6-(tetrahydro-2H-pyran-4-yl)piperazin-2-one (456)

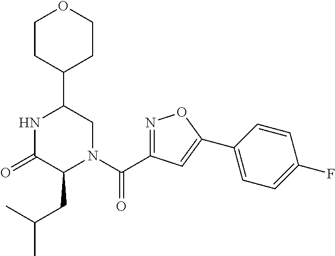

### Step 1: Synthesis of (S)-3-isobutyl-6-(tetrahydro-2H-pyran-4-yl)piperazin-2-one (457)

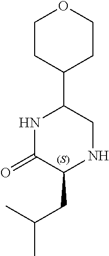

Synthesized from Fmoc-2-amino-2-(tetrahydro-2H-pyran-4-yl)acetic acid (11.98 g, 31.4 mmol) and (S)-methyl 2-amino-4-methylpentanoate (3.05 g, 16.4 mmol) by the method described for the compound 7 (Scheme II) to afford the product 457 (0.502 g, overall yield: 6.70%).

¹H NMR (400 MHz, CDCl₃) δ 6.12-6.35 (m, 1H), 3.94-4.09 (m, 2H), 2.94-3.38 (m, 5H), 2.00-2.01 (m, 1H), 1.20-1.86 (m, 9H), 0.87-0.93 (m, 6H).

MS (ESI): m/z 241.17 (M+H)⁺

### Step 2

Compound 457 (36 mg, 0.15 mmol) and 5-(4-fluorophenyl)isoxazole-3-carboxylic acid (35 mg, 0.17 mmol) were coupled according to the procedure described for compound 71 to furnish 456 (10 mg, 15.5% yield) as a white solid.

¹H NMR (400 MHz, CDCl₃): δ 7.80-7.77 (m, 2H), 7.21-7.10 (m, 2H), 6.88 (s, 1H), 6.17 (brs, 1H), 5.34-5.27 (m, 1H), 4.99 and 4.82 (dd, J=13.2 & 4.4 Hz, 1H), 4.06-4.00 (m, 2H), 3.59-3.47 (m, 1H), 3.43-3.34 (m, 2H), 3.23 and 2.95 (dd, J=13.6 & 11.2 Hz, 1H), 1.90-1.42 (m, 8H) and 1.08-0.78 (m, 6H).

MS (ESI): m/z 430.1 (M+1)⁺

### Example 402

### (3R,6S)-4-(5-(4-fluorophenyl)isoxazole-3-carbonyl)-3-((methylthio)ethyl)-6-phenylpiperazin-2-one (458)

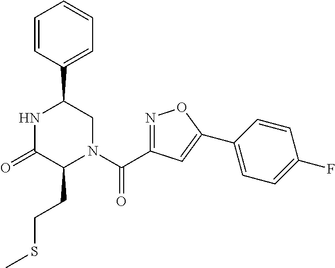

### Step 1: Synthesis of (3S,6S)-3-(2-(methylthio)ethyl)-6-phenylpiperazin-2-one (459)

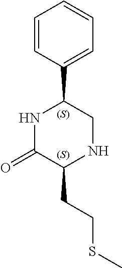

Synthesized from FMOC-L-2-Phenylglycine (12 g, 29.26 mmol) and (S)-methyl 2-amino-4-(methylthio)butanoate (3.6 g, 22.40 mmol) by the method described for the compound 7 (Scheme II) to afford the product 459 (1.097 g, overall yield: 13.7%).

¹H NMR (400 MHz, CDCl₃): δ 7.38-7.40 (m, 2H), 7.30-7.36 (m, 1H), 7.25-7.28 (m, 2H), 6.07 (s, 1H), 4.60-4.63 (m, 1H), 3.61-3.64 (m, 1H), 3.25-3.30 (m, 1H), 2.97-3.01 (m, 1H), 2.61-2.73 (m, 2H), 2.28-2.36 (m, 1H), 2.11 (s, 3H), 1.92-2.00 (m, 1H).

MS (ESI): 250.98 (M+H)⁺

### Step 2

Compound 459 (56 mg, 0.22 mmol) and 5-(4-fluorophenyl)isoxazole-3-carboxylic acid (45 mg, 0.22 mmol) were coupled according to the procedure described for compound 71 to furnish 458 (85 mg, 86.5% yield) as a white solid.

¹H NMR (400 MHz, CDCl₃): δ 7.81-7.77 (m, 2H), 7.43-7.37 (m, 5H), 7.20-7.16 (m, 2H), 6.88 (s, 1H), 6.44 (brs, 1H), 5.44-5.30 (m, 1H), 4.98-4.74 (m, 2H), 3.34 and 3.06 (dd, J=14.0 & 11.2 Hz, 1H), 2.78-2.40 (m, 3H) and 2.32-2.13 (m, 4H).

MS (ESI): m/z 440.7 (M+1)⁺

### Example 403

### (3R,6S)-4-((1R,2R)-2-(4-fluorophenyl)cyclopropanecarbonyl)-3-((methylthio)-methyl)-6-phenylpiperazin-2-one(460)

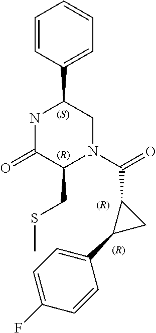

### Step 1: (3R,6S)-3-Methylsulfanylmethyl-6-phenyl-piperazin-2-one (461)

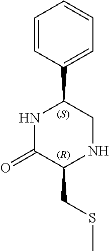

Synthesized from FMOC-L-2-Phenylglycine (23.41 g, 63 mmol) and 2-Amino-3-methylsulfanyl-propionic acid methyl ester hydrochloride (5.57 g, 30 mmol) by the method described for the compound 7 (Scheme II) to afford the product 461 (2.213 g, overall yield: 13.3%) as white solids.

¹H NMR (400 MHz, DMSO-d6) δ 8.08 (s, 1H), 7.24-7.39 (m, 5H), 4.50 (dd, J₁=7.6 Hz, J₂=4.0 Hz, 1H), 3.46-3.49 (m, 1H), 3.14 (dd, J₁=12.8 Hz, J₂=4.4 Hz, 1H), 2.85-2.89 (m, 3H), 2.09 (s, 3H).

MS (ESI): m/z 259.0827 [M+H]⁺

### Step 2

According to the method described for compound 89, compound 461 (54.7 mg, 0.23 mmol) and 60 (50 mg, 0.28 mmol) were coupled to give the product of 460 (60.5 mg, 66% yield) as a white solid.

¹H NMR (400 MHz, CDCl3): δ 7.41-7.33 (5H, m), 7.09-7.05 (2H, m), 7.01-6.92 (2H, m), 6.31 (1H, s), 4.85-4.79 (1H, m), 4.69-4.64 (1H, m), 3.24-3.17 (2H, m), 3.02 (1H, dd, J=10.8, 13.6 Hz), 2.60-2.49 (1H, m), 2.15 (3H, s), 1.94-1.90 (1H, m), 1.78-1.74 (1H, m), 1.70-1.66 (1H, m), 1.35-1.31 (1H, m).

MS (ESI): m/z 399.0 [M+1]⁺.

### Example 404

### (3S,6S)-3-allyl-4-((1R,2R)-2-(4-fluorophenyl)cyclopropanecarbonyl)-6-phenyl-piperazin-2-one(462)

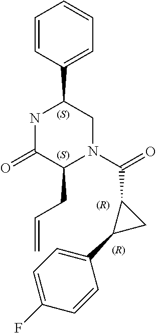

### Step 1: Synthesis of (3S,6S)-3-allyl-6-phenylpiperazin-2-one (463)

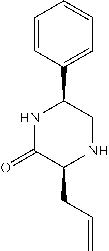

Synthesized from FMOC-L-2-Phenylglycine (23.41 g, 63 mmol) and (S)-methyl 2-aminopent-4-enoate (6.0 g, 35 mmol) by the method described for the compound 7 (Scheme II) to afford the product 463 (2.0 g, overall yield: 4.6%).

¹H NMR (400 MHz, DMSO-d₆) δ 7.96 (s, 1H), 7.24-7.36 (m, 5H), 5.81-5.91 (m, 1H), 5.02-5.09 (m, 2H), 4.46 (s, 1H), 3.28-3.31 (m, 1H) 3.08-3.09 (m, 1H), 2.79-2.84 (m, 1H), 2.39-2.44 (m, 1H), 2.24 (s, 1H).

MS (ESI): m/z 217.0 [M+H]⁺

### Step 2

According to the method described for compound 89, compound 463 (50 mg, 0.23 mmol) and 60 (50 mg, 0.28 mmol) were coupled to give the product 462 (57.9 mg, 66.5% yield) as a colorless oil.

¹H NMR (400 MHz, CDCl3): δ 7.43-7.32 (5H, m), 7.08-6.92 (4H, m), 6.22 (1H, s), 5.94-5.83 (1H, m), 5.29-5.18 (1H, m), 5.13-5.07 (1H, m), 4.82 (1H, dd, J=4, 14 Hz), 4.71-4.61 (2H, m), 2.92-2.66 (2H, m), 2.56-2.48 (1H, m), 1.91-1.86 (1H, m), 1.72-1.66 (1H, m), 1.34-1.23 (2H, m).

MS (ESI): m/z 379.1 [M+H]⁺

### Example 405

### (3S,6S)-6-cyclobutyl-4-((1R,2R)-2-(4-fluorophenyl)cyclopropanecarbonyl)-3-isobutylpiperazin-2-one(464)

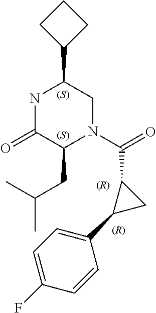

According to the method described for compound 89, compound 444 (48.4 mg, 0.23 mmol) and 60 (50 mg, 0.28 mmol) were coupled to give the product 464 (77 mg, 90% yield) as a colorless gum.

¹H NMR (400 MHz, CDCl3): δ 7.10-6.94 (4H, m), 5.97 (1H, s), 4.69-4.61 (1H, m), 4.56-4.48 (1H, m), 3.50-3.41 (1H, m), 2.54-2.48 (1H, m), 2.33-2.21 (1H, m), 2.12-1.54 (12H, m), 1.29-1.23 (1H, m), 1.02-0.92 (6H, m).

MS (ESI): m/z 373.1 [M+H]⁺

### Example 406

### (3S,6S)-3-allyl-4-(5-(4-fluorophenyl)-1,2,4-oxadiazole-3-carbonyl)-6-phenyl-piperazin-2-one(465)

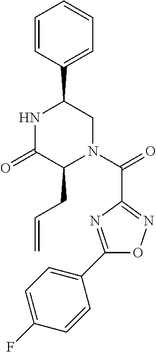

According to the method described for compound 89, compound 463 (51.9 mg, 0.24 mmol) and 66 (50 mg, 0.24 mmol) were coupled to give the product 465 (29.9 mg, 31% yield) as a colorless gum.

¹H NMR (400 MHz, CDCl3): δ 8.24-8.19 (2H, m), 7.47-7.33 (5H, m), 7.29-7.23 (2H, m), 6.12 (1H, s), 6.02-5.92 (1H, m), 5.22-5.15 (3H, m), 4.88 (1H, dd, J=4, 11 Hz), 4.42 (1H, dd, J=4, 14.4 Hz), 3.45 (1H, dd, J=11, 14 Hz), 2.88-2.81 (2H, m).

MS (ESI): m/z 407.0 [M+H]⁺.

### Example 407

### (3R,6S)-4-(5-(4-fluorophenyl)-1,2,4-oxadiazole-3-carbonyl)-3-((methylthio)methyl)-6-phenylpiperazin-2-one(466)′

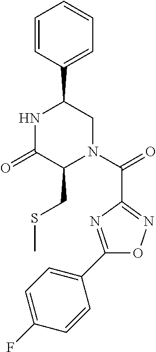

According to the method described for compound 89, compound 461 (56.7 mg, 0.24 mmol) and 66 (50 mg, 0.24 mmol) were coupled to give the product 466 (42.2 mg, 41% yield) as a white solid.

¹H NMR (400 MHz, CDCl3): δ 8.24-8.19 (2H, m), 7.44-7.36 (5H, m), 7.27-7.22 (2H, m), 6.36 (1H, s), 5.42-5.39 (1H, m), 4.88 (1H, dd, J=4, 11 Hz), 4.50 (1H, dd, J=4, 14.4 Hz), 3.82 (1H, dd, J=11, 14 Hz), 3.33-3.19 (2H, m), 2.27 (3H, s).

MS (ESI): m/z 427.0 [M+H]⁺.

### Example 408

### (3S,6S)-6-cyclobutyl-4-(5-(4-fluorophenyl)-1,2,4-oxadiazole-3-carbonyl)-3-isobutylpiperazin-2-one(467)

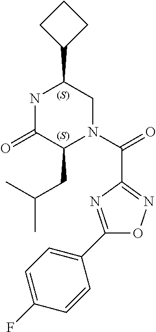

According to the method described for compound 89, compound 444 (50 mg, 0.24 mmol) and 66 (50 mg, 0.24 mmol) were coupled to give the product 467 (38.9 mg, 40% yield) as a colorless solid.

¹H NMR (400 MHz, CDCl3): δ 8.23-8.18 (2H, m), 7.27-7.22 (2H, m), 6.04 (1H, s), 5.29-5.26 (1H, m), 4.85-4.80 (1H, m), 4.31-4.25 (1H, m), 3.02 (1H, dd, J=11, 14 Hz), 2.16-1.67 (10H, m), 1.09 (3H, d, J=6 Hz), 0.98 (3H, d, J=6 Hz).

MS (ESI): m/z 401.1 [M+1]⁺

### Example 409

### (3R,6S)-4-(5-(4-fluorophenyl)isoxazole-3-carbonyl)-3-((methylthio)methyl)-6-phenylpiperazin-2-one (468)

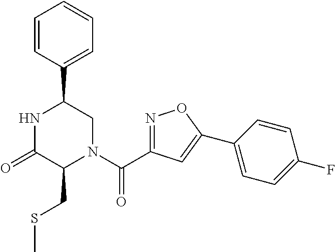

Compound 461 (50 mg, 0.21 mmol) and 5-(4-fluorophenyl)isoxazole-3-carboxylic acid (45 mg, 0.22 mmol) were coupled according to the procedure described for compound 71 to furnish 468 (40 mg, 44.4% yield) as a white solid.

¹H NMR (400 MHz, CDCl₃): δ 7.81-7.77 (m, 2H), 7.44-7.38 (m, 5H), 7.21-7.16 (m, 2H), 6.88 (s, 1H), 6.30 (brs, 1H), 5.63 and 5.39 (t, J=5.6 Hz, 1H), 5.00-4.75 (m, 2H), 3.70 (dd, J=14.0 & 11.2 Hz, 0.5H), 3.31-3.19 (m, 2.5H) and 2.79-2.76 (s, 3H).

MS (ESI): m/z 440.7 (M+H)⁺.

### Example 410

### (3R,6S)-4-((1R,2R)-2-(4-fluorophenyl)cyclopropanecarbonyl)-6-phenyl-3-(thiophen-2-yl)piperazin-2-one (469)

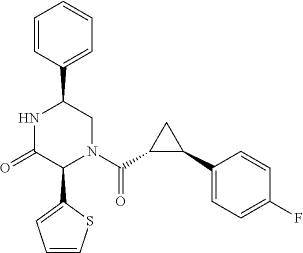

### Step 1: Synthesis of (3R,6S)-6-Phenyl-3-thiophen-2-yl-piperazin-2-one (470)

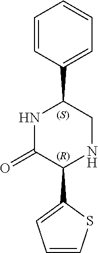

Synthesized from FMOC-L-2-Phenylglycine (7.80 g, 21 mmol) and (R)-Amino-thiophen-2-yl-acetic acid methyl ester hydrochloride (2.1 g, 10.14 mmol) by the method described for the compound 7 (Scheme II) to afford the product 470 (250.10 mg, overall yield: 2.7%) as a white solid.

¹H NMR (400 MHz, DMSO-d6): δ 8.17 (s, 1H), 7.23-7.37 (m, 6H), 7.07-7.08 (m, 1H), 6.94-6.97 (m, 1H), 4.63 (s, 1H), 4.55-4.57 (m, 1H), 3.04-3.09 (m, 1H), 2.63-2.68 (m, 1H).

MS (ESI): m/z 258.87 [M+H]⁺

### Step 2

Compound 470 (50 mg, 0.19 mmol) and 60 (40 mg, 0.21 mmol) were coupled according to the procedure described for compound 71 to furnish compound 469 (71 mg, 87% yield) as a colorless solid.

¹H NMR (400 MHz, CDCl₃): δ 7.40-7.25 (m, 5H), 7.17-6.87 (m, 6H), 6.52-6.38 (m, 2H), 5.93 (s, 1H), 4.85-4.70 (m, 2H), 3.51-2.91 (m, 1H), 2.63-2.52 (m, 1H), 1.97-1.92 (m, 1H), 1.81-1.54 (m, 1H), 1.40-1.31 (m, 1H).

MS (ESI): m/z 421.0 [M+H]⁺

### Example 411

### (3R,6S)-4-(5-(4-fluorophenyl)isoxazole-3-carbonyl)-6-phenyl-3-(thiophen-2-yl)piperazin-2-one (471)

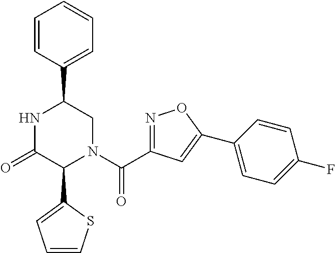

Compound 470 (52 mg, 0.20 mmol) and 5-(4-fluorophenyl)isoxazole-3-carboxylic acid (45 mg, 0.22 mmol) were coupled according to the procedure described for compound 71 to furnish 471 (45 mg, 50% yield) as a white solid.

¹H NMR (400 MHz, CDCl₃): δ 7.82-7.78 (m, 2H), 7.43-7.26 (m, 6H), 7.24-7.18 (m, 4H), 7.04-7.01 (m, 1H), 6.94 and 6.89 (brs, 1H), 6.24 and 6.16 (brs, 1H), 5.07-4.84 (m, 2H), 3.47 and 3.20 (dd, 14.0 & 4.0 Hz, 1H) and 1.58 (s, 1H).

MS (ESI): m/z 448.05 (M+H)⁺.

### Example 412

### (3R,6S)-4-(5-(4-fluorophenyl)-1,2,4-oxadiazole-3-carbonyl)-6-phenyl-3-(thiophen-2-yl)piperazin-2-one (472)

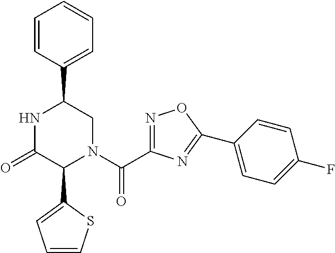

Compound 470 (50 mg, 0.19 mmol) and 66 (42 mg, 0.21 mmol) were coupled according to the procedure described for compound 71 to furnish compound 472 (45 mg, 52% yield) as a colorless solid.

¹H NMR (400 MHz, CDCl₃): δ 8.24-8.19 (m, 2H), 7.44-7.32 (m, 6H), 7.32-7.22 (m, 3H), 7.04-7.01 (m, 1H), 6.61-6.40 (m, 2H), 5.05-4.43 (m, 2H), 3.54-3.18 (m, 1H).

MS (ESI): m/z 448.9 [M+H]⁺

### Example 413

### (3S,6R)-4-(5-(4-fluorophenyl)-1,2,4-oxadiazole-3-carbonyl)-3-isobutyl-6-(tetrahydro-2H-pyran-4-yl)piperazin-2-one (473) and (3S,6S)-4-(5-(4-fluorophenyl)-1,2,4-oxadiazole-3-carbonyl)-3-isobutyl-6-(tetrahydro-2H-pyran-4-yl)piperazin-2-one (474)

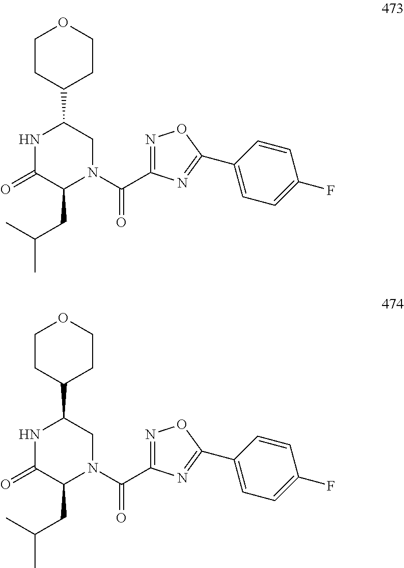

Compound 457 (96 mg, 0.4 mmol) and 66 (90 mg, 0.44 mmol) were coupled according to the procedure described for compound 71 to furnish 473 (30 mg, 34.9% yield) and 474 (20 mg, 23.3% yield) as white solids.

### Compound 476

¹H NMR (400 MHz, CDCl₃): δ 8.23-8.19 (m, 2H), 7.28-7.23 (m, 2H), 6.35 (dd, J=9.6 & 3.2 Hz, 1H), 5.30 and 4.97 (q, J=4.4 Hz, 1H), 4.86 and 4.33 (dd, J=14.4 Hz, 1H), 4.05-3.97 (m, 1.5H), 3.80-3.68 (m, 1H), 3.39-3.31 (m, 2H), 3.24-3.16 (m, 1H), 3.04-3.01 (m, 0.5H), 2.00-1.64 (m, 5H), 1.42-1.26 (m, 2H), 1.18-1.12 (m, 1H) and 1.07-0.69 (m, 6H).

MS (ESI): m/z 431.1 (M+H)⁺

### Compound 477

¹H NMR (400 MHz, CDCl₃): δ 8.23-8.19 (m, 2H), 7.28-7.23 (m, 2H), 6.35 (dd, J=9.6 & 3.2 Hz, 1H), 5.30 and 4.97 (q, J=4.4 Hz, 1H), 4.86 and 4.33 (dd, J=14.4 Hz, 1H), 4.05-3.97 (m, 1.5H), 3.80-3.68 (m, 1H), 3.39-3.31 (m, 2H), 3.24-3.16 (m, 1H), 3.04-3.01 (m, 0.5H), 2.00-1.64 (m, 5H), 1.42-1.26 (m, 2H), 1.18-1.12 (m, 1H) and 1.07-0.69 (m, 6H).

MS (ESI): m/z 431.1 (M+H)⁺

### Example 414

### (3S,6R)-4-((1R,2R)-2-(4-fluorophenyl)cyclopropanecarbonyl)-3-isobutyl-6-(tetrahy-dro-2H-pyran-4-yl)piperazin-2-one (475) and (3S,6S)-4-((1R,2R)-2-(4-fluoro-phenyl)cyclopropanecarbonyl)-3-isobutyl-6-(tetrahydro-2H-pyran-4-yl)piperazin-2-one (476)

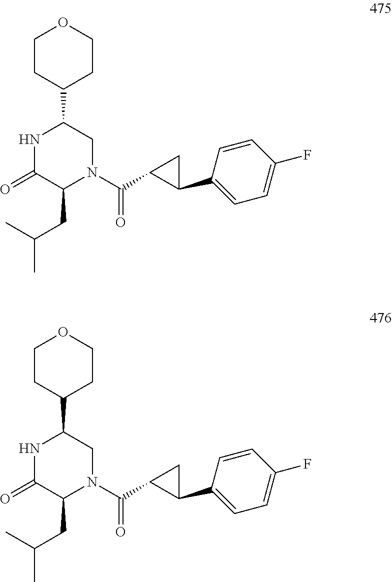

Compound 457 (72 mg, 0.30 mmol) and 60 (64 mg, 0.34 mmol) were coupled according to the method described for the preparation of compound 71 to furnish product 475 (45 mg, 74.6% yield) and 476 (55 mg, 90.1% yield) as a white solid.

### Compound 475

¹H NMR (400 MHz, CDCl₃): δ 7.09-6.94 (m, 4H), 6.71 (s, 1H), 5.17 and 4.53 (dd, J=8.8 & 4.4 Hz, 1H), 4.74 (dd, J=13.2 & 4.4 Hz, 0.6H), 4.07-3.96 (m, 2.4H), 3.39-3.32 (m, 3H), 3.19 and 2.71 (dd, J=14.0 & 11.2 Hz, 1H), 2.54-2.48 (m, 1H), 1.88-1.38 (m, 10H), 1.28-1.23 (m, 1H) and 1.00-0.92 (m, 6H).

MS (ESI): m/z 403.1 [M+H]⁺

### Compound 476

¹H NMR (400 MHz, CDCl₃): δ 7.08-6.96 (m, 4H), 6.50 (s, 1H), 5.18 and 4.54 (dd, J=8.8 & 4.8 Hz, 1H), 4.68 (d, J=13.4 Hz, 0.6H), 4.10-3.96 (m, 2.4H), 3.59 and 3.13 (dd, J=14.4 & 4.0 HZ, 1H), 3.38-3.28 (m, 2H), 3.07-3.03 (m, 1H), 2.70-2.51 (m, 1H), 1.92-1.24 (m, 11H) and 1.01-0.94 (m, 6H).

MS (ESI): m/z 403.1 [M+H]⁺

### Example 415

### Synthesis of (3S,6R)-6-(furan-2-yl)-3-isobutylpiperazin-2-one (477) and (3S,6S)-6-(furan-2-yl)-3-isobutylpiperazin-2-one (478)

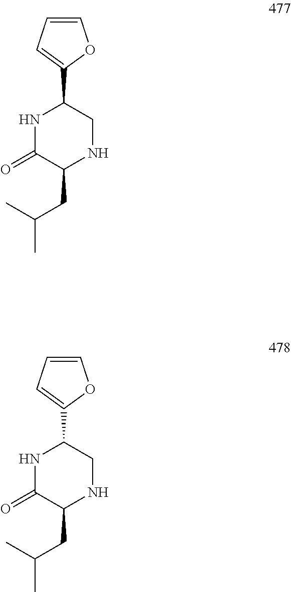

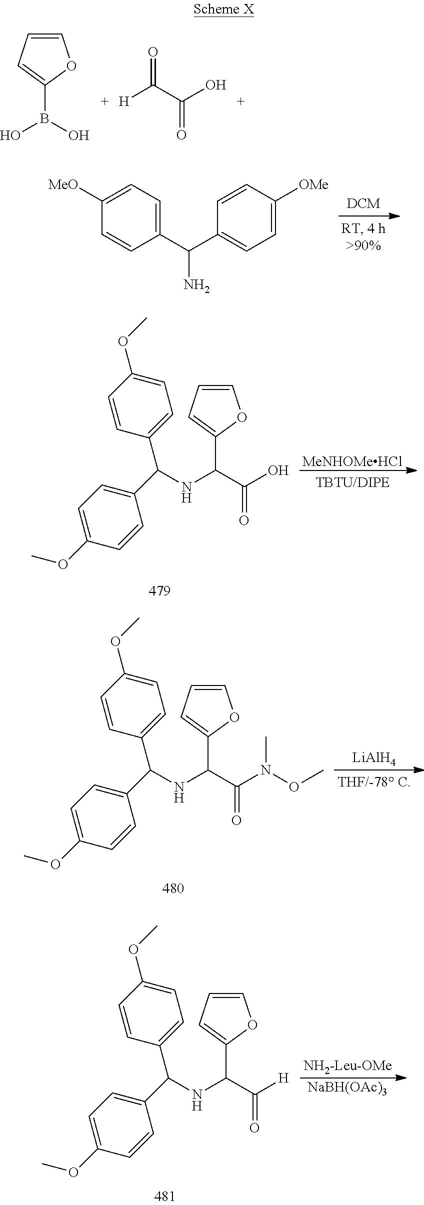

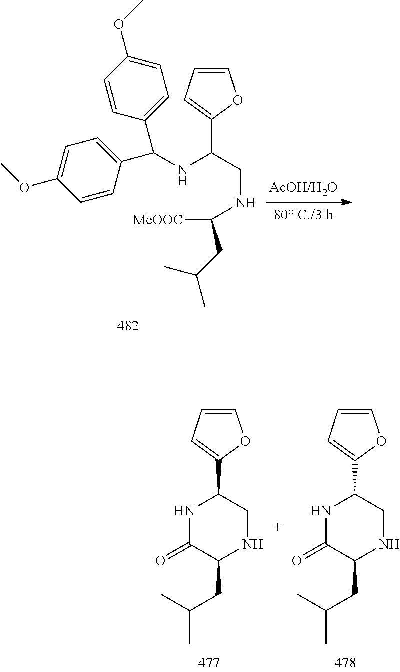

### Step 1: 2-((bis(4-methoxyphenyl)methyl)amino)-2-(furan-2-yl)acetic acid (479)

To a stirred solution of glyoxylic acid monohydrate (920 mg, 10.11 mmol) in CH₂Cl₂ (70 mL) were added bis(4-methoxyphenyl)methanamine (2.46 g, 10.11 mmol) and 2-furanboronic acid (1.13 g, 10.11 mmol) at once. The reaction mixture was stirred at room temperature for 2 minutes to form clear solution. The solution was purged with Ar (2 min) and the sealed reaction mixture was stirred for over night. The solvent was concentrated to dryness to yield compound 479 (3.5 g of crude product, 94% yield) as pale yellow foam. The crude product was used for next step with out purification.

¹H NMR (400 MHz, CDCl₃): δ 7.31 (s, 1H), 7.27-7.24 (m, 4H), 6.81-6.79 (m, 4H), 6.28 (brs, 1H), 6.24 (brs, 1H), 4.80 (s, 1H), 4.44 (s, 1H) 3.76 (s, 3H), 3.75 (s, 3H) 3.70 (s, 1H).

MS (ESI): m/z 368.0 [M+1]⁺

### Step 2: 2-((bis(4-methoxyphenyl)methyl)amino)-2-(furan-2-yl)-N-methoxy-N-methyl-acetamide (480)

Compound 479 (0.5 g, 1.36 mmol), TBTU (0.66 g, 2.04 mmol), dimethylhydroxylamine hydrochloride (0.2 g, 2.04 mmol) were combined and added dry CH₃CN (6 mL) and di-isopropylethylamine (710 uL, 4.08 mmol). This mixture was stirred at room temperature for 4 h. The solvent was removed under reduced pressure and the crude product was purified by column chromatography using EtOAc/Hex (0 to 40%) to afford compound 480 as a foam (520 mg, 93% yield).

¹H NMR (400 MHz, CDCl₃): δ 7.37 (s, 1H), 7.34-7.25 (m, 4H), 6.85-6.79 (m, 4H), 6.33-6.32 (m, 1H), 6.22 (d, J=3.2 Hz, 1H), 4.83 (s, 1H), 4.68 (s, 1H) 3.77 (s, 3H), 3.75 (s, 3H) 3.28 (s, 3H), 3.20 (s, 3H).

MS (ESI): m/z 411.1 [M+H]⁺

### Step 3: 2-((bis(4-methoxyphenyl)methyl)amino)-2-(furan-2-yl)acetaldehyde (481)

To a solution of compound 480 (0.2 g, 1.22 mmol) in dry THF (3 mL) was cooled to −78° C. and added LiAlH₄ (40 mg, 0.97 mmol) at once. The reaction mixture was stirred for 3 h at −78° C. under Ar atmosphere. The reaction was quenched with saturated aqueous NH₄Cl solution by drop wise addition. This mixture was partitioned between EtOAc/Brine, and extracted with EtOAc. The EtOAc layers were combined and washed with brine, and dried (MgSO₄). The crude compound 481 (unstable, slowly decomposes at room temperature) was dried and used for next step.

### Step 4: (2S)-methyl 2-((2-((bis(4-methoxyphenyl)methyl)amino)-2-(furan-2-yl)ethyl)amino)-4-methylpentanoate (482)

To a solution of compound 481 (0.4 g, 1.14 mmol) and L-Leu-OMe-HCl (210 mg, 1.14 mmol) in dichloromethane (8 mL), NaBH(OAc)₃ (340 mg, 1.59 mmol) was added and the mixture was stirred at room temperature under Ar atmosphere for 6 h. The reaction mixture was quenched with saturated aqueous NaHCO₃ solution (30 mL) and the aqueous solution was extracted with EtOAc (30 ml) and dried (Na₂SO₄). The solvent was concentrated to dryness and the crude product was purified by column chromatography using EtOAc/Hex (0 to 40%) to afford compound 482 as pale yellow oil (0.27 g, 49% yield).

¹H NMR (400 MHz, CDCl₃): δ 7.37-7.36 (m, 1H), 7.30-7.17 (m, 4H), 6.85-6.76 (m, 4H), 6.32-6.31 (m, 1H), 6.16-6.11 (m, 1H), 4.64 (d, J=5.2 Hz, 1H), 3.78-3.67 (m, 7H) 3.28-3.20 (m, 1H), 2.94-2.88 (m, 1H) 2.75-2.66 (m, 1H), 1.96 (s, 1H), 1.70-1.64 (m, 1H), 1.48-1.40 (m, 2H), 0.94-0.86 (m, 6H).

MS (ESI): m/z 481.1 [M+H]⁺

### Step 5: (3S,6R)-6-(furan-2-yl)-3-isobutylpiperazin-2-one (477) and (3S,6S)-6-(furan-2-yl)-3-isobutylpiperazin-2-one (478)

Compound 482 (0.4 g, 0.84 mmol), was dissolved in 70% aqueous AcOH (5 mL) and heated under reflux (80° C.) for 3 h. The solvent was removed under reduced pressure and the crude product was purified by column chromatography using EtOAc/Hex, (20-60%) to afford compounds 477 and 478 (Over all yield 160 mg, 75%) as an oils.

### Compound 477

¹H NMR (400 MHz, CDCl₃): δ 7.39-7.38 (m, 1H), 6.40 (s, 1H), 6.45-6.34 (m, 1H), 6.26-6.25 (m, 1H), 4.63-4.61 (m, 1H), 3.46-3.43 (m, 1H), 3.24-3.23 (m, 2H), 1.91 (s, 1H), 1.89-1.79 (m, 2H), 1.53-1.47 (m, 1H) 0.95-0.82 (m, 6H).

MS (ESI): m/z 223.0 [M+H]⁺

### Compound 478

¹H NMR (400 MHz, CDCl₃): δ 7.38-7.37 (m, 1H), 6.34-6.33 (m, 1H), 6.26-6.25 (m, 1H), 6.07 (s, 1H), 4.68 (m, 1H), 3.75 (d, J=7.2 Hz, 1H), 3.47-3.43 (m, 1H), 3.40-3.35 (m, 2H), 3.06-3.01 (m, 1H), 1.89-1.78 (m, 2H), 1.60-1.54 (m, 1H) 0.97-0.92 (m, 6H).

MS (ESI): m/z 223.0 [M+H]⁺

### Example 416

### (3S,6S)-4-(5-(4-fluorophenyl)isoxazole-3-carbonyl)-6-(furan-2-yl)-3-isobutyl-piperazin-2-one (483)

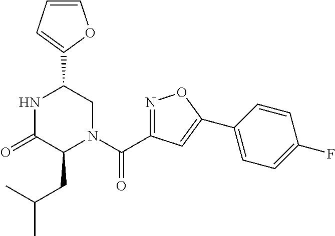

Compound 478 (40 mg, 0.18 mmol) and 5-(4-fluorophenyl)isoxazole-3-carboxylic acid (55 mg, 0.3 mmol) were coupled according to the procedure described for compound 71 to afford compound 483 as a colorless solid (16 mg, 22% yield).

¹H NMR (400 MHz, CDCl₃): δ 7.22-7.77 (m, 2H), 7.45-7.42 (m, 1H), 7.21-7.17 (m, 2H), 6.89 (s, 1H), 6.40-6.38 (m, 2H), 6.25 (s, 1H), 5.43-5.30 (m, 1H), 5.07-4.85 (m, 2H), 3.67-3.34 (m, 1H), 1.96-1.84 (m, 2H), 1.80-1.69 (m, 1H) 1.10-0.80 (m, 6H).

MS (ESI): m/z 412.1 [M+H]⁺

### Example 417

### (3S,6R)-4-(5-(4-fluorophenyl)isoxazole-3-carbonyl)-6-(furan-2-yl)-3-isobutylpiperazin-2-one (484)

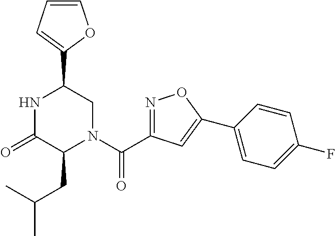

Compound 477 (40 mg, 0.18 mmol) and 5-(4-fluorophenyl)isoxazole-3-carboxylic acid (55 mg, 0.3 mmol) were coupled according to the procedure described for compound 71 to afford compound 484 (17 mg, 24% yield) as an colorless gum.

¹H NMR (400 MHz, CDCl₃): δ 7.72-7.69 (m, 2H), 7.19-7.15 (m, 2H), 7.11-7.10 (m, 1H), 6.71-6.60 (m, 1H), 6.30 (s, 1H), 6.26-6.22 (m, 2H), 5.45-5.40 (m, 1H), 5.01-4.95 (m, 1H), 4.76-4.64 (m, 1H), 3.93-3.54 (m, 1H), 1.90-1.86 (m, 2H), 1.75-1.69 (m, 1H) 1.05-0.74 (m, 6H).

MS (ESI): m/z 412.1 [M+1]⁺

### Example 418

### (3S,6S)-6-(5-chlorothiophen-2-yl)-4-(5-(4-fluorophenyl)isoxazole-3-carbonyl)-3-isobutylpiperazin-2-one (485)

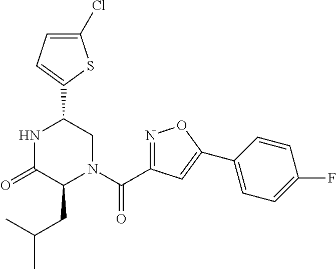

### Step 1: Synthesis of (3S,6S)-6-(5-chlorothiophen-2-yl)-3-isobutylpiperazin-2-one (486) and (3S,6R)-6-(5-chlorothiophen-2-yl)-3-isobutylpiperazin-2-one (487)

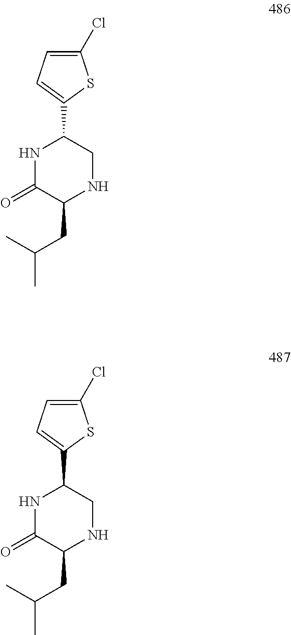

Synthesized from (5-chlorothiophen-2-yl)boronic acid (1.62 g, 10 mmol) and L-Leu-OMe-HCl (420 mg, 2.28 mmol) by the method described for the compound 477 and 478 (Scheme X) to afford the product 486 and 487 (250.10 mg, overall yield: 2.7%) as a brown solid.

### Compound 486

¹H NMR (400 MHz, CDCl₃): δ 6.85 (s, 1H), 6.78 (d, J=4.8 Hz, 1H), 6.69 (d, J=4.8 Hz, 1H), 4.72-4.68 (m, 1H), 3.43 (dd, J=10.4 & 2.8 Hz, 1H), 3.26 (dd, J=12.8 & 4.0 Hz, 1H), 3.07 (dd, J=12.8 & 2.8 Hz, 1H), 1.82-1.52 (m, 3H) and 0.96-0.86 (m, 6H).

MS (ESI): m/z 273.0 [M+H]⁺

### Compound 487

¹H NMR (400 MHz, CDCl₃): δ 6.78 (m, 2H), 6.43 (s, 1H), 4.73 (q, J=4.0 Hz, 1H), 3.43 (dd, J=10.0 & 3.6 Hz, 1H), 3.35 (dd, J=12.8 & 4.8 Hz, 1H), 2.88 (dd, J=12.8 & 8.8 Hz, 1H), 1.84-1.70 (m, 2H), 1.58-1.54 (m, 1H) and 0.97-0.88 (m, 6H).

MS (ESI): m/z 273.0 [M+H]⁺

### Step 2

Compound 486 (28 mg, 0.10 mmol) and 5-(4-fluorophenyl)-isoxazole-3-carboxylic acid (25 mg, 0.12 mmol) were coupled according to the method described for the preparation of compound 71 to furnish product 485 (20 mg, 42.2% yield) as a white solid.

¹H NMR (400 MHz, CDCl₃): δ 7.73-7.70 (m, 2H), 7.19-7.15 (m, 2H), 6.68-6.62 (m, 3H), 6.27 (brs, 1H), 5.46 (q, J=4.8 Hz, 1H), 4.86 (d, J=14.4 Hz, 1H), 3.99 (dd, J=14.8 & 4.0 Hz, 1H), 1.92-1.72 (m, 3H), 1.36-1.22 (m, 1H) and 1.06-0.73 (m, 6H).

MS (ESI): m/z 462.2 [M+H]⁺

### Example 419

### (3S,6R)-6-(5-chlorothiophen-2-yl)-4-(5-(4-fluorophenyl)isoxazole-3-carbonyl)-3-isobutylpiperazin-2-one (488)

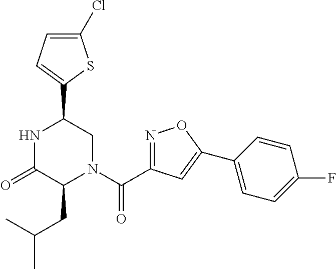

Compound 487 (55 mg, 0.20 mmol) and 5-(4-fluorophenyl)-isoxazole-3-carboxylic acid (45 mg, 0.22 mmol) were coupled according to the method described for the preparation of compound 71 to furnish product 488 (60 mg, 64.4% yield) as a white solid.

¹H NMR (400 MHz, CDCl₃): δ 7.80-7.77 (m, 2H), 7.18 (t, J=8.4 Hz, 2H), 6.92-6.80 (m, 3H), 6.73 (brs, 1H), 5.42-5.28 (m, 1H), 5.07-4.91 (m, 2H), 3.42 and 3.15 (dd, J=13.2 & 10.0 Hz, 1H), 1.88-1.67 (m, 3H) and 1.08-0.79 (m, 6H).

MS (ESI): m/z 462.2 [M+H]⁺

### Example 420

### (3S,6R)-6-(5-chlorothiophen-2-yl)-4-(5-(4-fluorophenyl)isoxazole-3-carbonyl)-3-isobutylpiperazin-2-one (489)

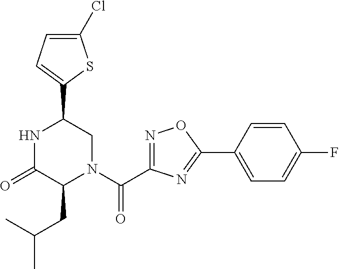

Compound 487 (40 mg, 0.10 mmol) and 66 (35 mg, 0.17 mmol) were coupled according to the method described for the preparation of compound 71 to furnish product 489 (50 mg, 73.7% yield) as a white solid.

¹H NMR (400 MHz, CDCl₃): δ 8.23-8.18 (m, 2H), 7.28-7.23 (m, 2H), 6.92-6.81 (m, 2H), 6.63 (brs, 1H), 5.34-5.30 (m, 0.7H), 5.11-4.92 (m, 1.6H), 4.50-4.45 (m, 0.7H), 3.45 and 3.18 (dd, J=14.4 & 11.2 Hz, 1H), 1.92-1.68 (m, 3H) and 1.10-0.74 (m, 6H).

MS (ESI): m/z 465.01 [M+2H]⁺

### Example 421

### (3S,6R)-6-(5-chlorothiophen-2-yl)-4-(5-(4-fluorophenyl)isoxazole-3-carbonyl)-3-isobutylpiperazin-2-one (490)

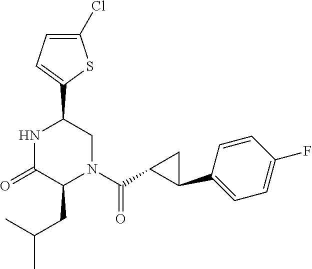

Compound 487 (27 mg, 0.10 mmol) and trans (S,S)[2-(4-fluoro)phenyl]-cyclopropyl-1-carboxylic acid 60 (20 mg, 0.11 mmol) were coupled according to the method described for the preparation of compound 71 to furnish product 490 (32 mg, 74.3% yield) as a white solid.

¹H NMR (400 MHz, CDCl₃): δ 7.08-6.94 (m, 4H), 6.88-6.80 (m, 2H), 6.37 (brs, 1H), 5.24 and 4.59 (dd, J=10.0 & 4.0 Hz, 1H), 4.86-4.80 (m, 1.6H), 4.23 (dd, J=14.0 & 4.0 Hz, 0.4H), 3.44 and 2.94 (dd, J=14.4 & 11.2 Hz, 1H), 2.57-2.50 (m, 1H), 1.92-1.64 (m, 5H), 1.34-1.26 (m, 1H) and 1.02-0.95 (m, 6H).

MS (ESI): m/z 435.1 [M+H]⁺

### Example 422

### (3S,6R)-4-(5-(4-fluorophenyl)isoxazole-3-carbonyl)-3-propyl-6-(thiophen-2-yl)piper-azin-2-one (491)

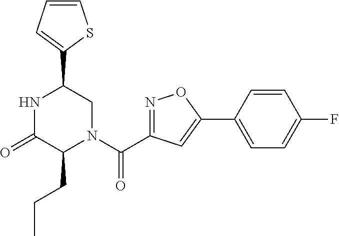

### Step 1: (3S,6R)-3-propyl-6-(thiophen-2-yl)piperazin-2-one (492) and (3S,6S)-3-propyl-6-(thiophen-2-yl)piperazin-2-one (493)

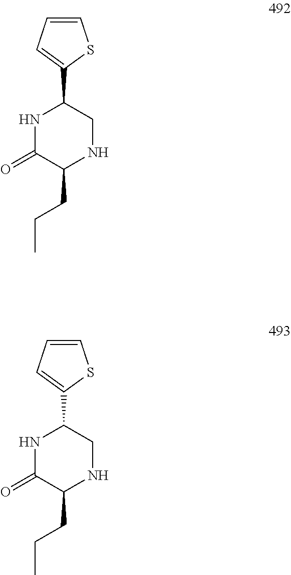

Synthesized from thiophene-2-boronic acid 1 (19.0 g, 0.1484 mol) and (S)-methyl 2-aminopentanoate hydrochloride 7 (7.1 g, 0.0425 mol) by the method described for the compound 477 and 478 (Scheme X) to afford 492 (1.331 g) and 493 (1.183 g) as a brown gum.

### Compound 492

¹H NMR (400 MHz, CDCl₃) δ 7.27-7.26 (m, 1H), 7.00-6.96 (m, 2H), 6.36 (s, 1H), 4.88-4.85 (m, 1H), 3.48-3.45 (m, 1H), 3.34-3.29 (m, 1H), 3.15-3.10 (m, 1H), 2.04-2.00 (m, 1H), 1.77-1.67 (m, 1H), 1.56-1.37 (m, 2H), 0.96-0.93 (t, J=6.0 Hz, 3H)

MS (ESI): m/z 224.93 [M+H]⁺

### Compound 493

¹H NMR (400 MHz, CDCl₃) δ 7.28-7.25 (m, 1H), 7.02-6.97 (m, 2H), 6.00 (s, 1H), 4.91-4.88 (m, 1H), 3.49-3.45 (m, 1H), 3.40-3.56 (m, 1H), 2.99-2.93 (m, 1H), 2.02-1.95 (m, 1H), 1.71-1.63 (m, 1H), 1.53-1.39 (m, 2H), 0.98-0.94 (t, J=8.0 Hz, 3H)

MS (ESI): m/z 224.95 [M+H]⁺

### Step 2

Compound 492 (70 mg, 0.31 mmol) and 5-(4-fluorophenyl)-isoxazole-3-carboxylic acid (70 mg, 0.34 mmol) were coupled according to the method described for the preparation of compound 71 to furnish product 491 (100 mg, 77.5% yield) as a white solid.

¹H NMR (400 MHz, CDCl₃): δ 7.81-7.76 (m, 2H), 7.34-7.31 (m, 1H), 7.20-7.12 (m, 3H), 7.04-7.00 (m, 1H), 6.87 (brs, 1H), 6.54 (brs, 1H), 5.39-5.23 (m, 1H), 5.17-4.93 (m, 2H), 3.47 and 3.18 (dd, J=14.0 & 11.2 Hz, 1H), 2.16-1.86 (m, 2H), 1.58-1.40 (m, 2H) and 1.01-0.89 (m, 3H).

MS (ESI): m/z 414.1 [M+H]⁺

### Example 423

### (3S,6R)-4-(5-(4-fluorophenyl)isoxazole-3-carbonyl)-3-propyl-6-(thiophen-2-yl)piperazin-2-one (494)

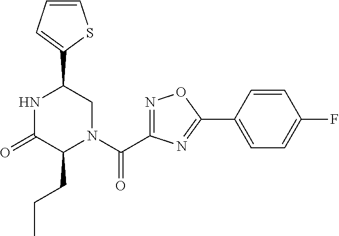

Compound 492 (45 mg, 0.20 mmol) and 66 (45 mg, 0.22 mmol) were coupled according to the method described for the preparation of compound 71 to furnish product 494 (72 mg, 86.6% yield) as a white solid.

¹H NMR (400 MHz, CDCl₃): δ 8.23-8.18 (m, 2H), 7.36-7.31 (m, 1H), 7.27-7.23 (m, 2H), 7.10 (dd, J=12.8 & 4.0 Hz, 1H), 7.04-6.99 (m, 1H), 6.36 (d, J=8.0 Hz, 1H), 5.28 and 4.94 (m, 1H), 5.20 and 5.12 (dd, J=10.8 & 4.0 Hz, 1H), 5.02 and 4.45 (m, 1H), 3.50 and 3.22 (dd, J=14.0 & 10.8 Hz, 1H), 2.20-1.88 (m, 2H), 1.64-1.40 (m, 2H) and 1.02-0.89 (m, 3H).

MS (ESI): m/z 415.1 [M+H]⁺

### Example 424

### (3S,6R)-4-((1R,2R)-2-(4-fluorophenyl)cyclopropanecarbonyl)-3-propyl-6-(thiophen-2-yl)piperazin-2-one (495)

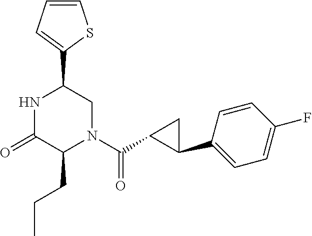

Compound 492 (45 mg, 0.20 mmol) and 60 (40 mg, 0.22 mmol) were coupled according to the method described for the preparation of compound 71 to furnish product 495 (52 mg, 67.1% yield) as a white solid.

¹H NMR (400 MHz, CDCl₃): δ 7.33-7.30 (m, 1H), 7.09-6.95 (m, 6H), 6.26 (brs, 1H), 5.20 and 4.59 (dd, J=9.2 & 4.0 Hz, 1H), 4.99-4.85 (m, 1.7H), 4.25 (dd, J=14.0 & 4.0 Hz, 0.3H), 3.48 and 2.97 (dd, J=14.4 & 11.2 Hz, 1H), 2.56-2.50 (m, 1H), 2.10-1.66 (m, 4H), 1.46-1.28 (m, 3H) and 1.01-0.95 (m, 3H).

MS (ESI): m/z 487.1 [M+H]⁺

### Example 425

### (3S,6S)-6-(2-chlorothiophen-3-yl)-4-(5-(4-fluorophenyl)isoxazole-3-carbonyl)-3-isobutylpiperazin-2-one (496)

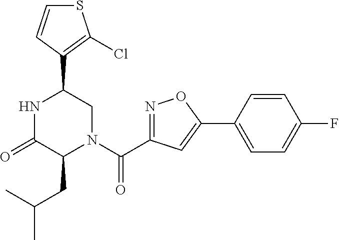

### Step 1: Synthesis of (3S,6S)-6-(2-chlorothiophen-3-yl)-3-isobutylpiperazin-2-one (497) and (3S,6R)-6-(2-chlorothiophen-3-yl)-3-isobutylpiperazin-2-one (498)

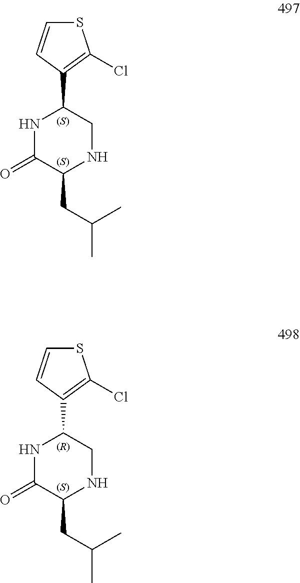

Synthesized from 2-chlorothiophen-3-ylboronic acid (4.15 g, 56.12 mmol) and (S)-methyl 2-amino-4-methylpentanoate hydrochloride (3.15 g, 17.39 mmol) by the method described for the compound 477 and 478 (Scheme X) to afford the product 497 (237 mg, overall yield: 1.6%) and product 498 (404 mg, overall yield: 2.6%).

### Compound 497

¹H NMR (400 MHz, DMSO-d₆): δ 7.13 (d, J=6.0 Hz, 1H), 6.99 (d, J=6.0 Hz, 1H), 5.91 (s, 1H), 4.78-4.75 (m, 1H), 3.82-3.74 (m, 1H), 3.54-3.50 (m, 1H), 3.26-3.21 (m, 1H), 3.10-3.06 (m, 1H), 1.88-1.80 (m, 2H), 1.64-1.54 (m, 1H), 0.99-0.94 (m, 6H)

MS (ESI): m/z 272.83 [M+H]⁺

### Compound 498

¹H NMR (400 MHz, DMSO-d₆) δ 7.14 (d, J=4.8 Hz, 1H), 6.96 (d, J=4.8 Hz, 1H), 5.82 (s, 1H), 4.86-4.82 (m, 1H), 3.77-3.75 (m, 1H), 3.52-3.48 (m, 1H), 3.34-3.30 (m, 1H), 2.89-2.84 (m, 1H), 1.99-1.92 (m, 1H), 1.86-1.78 (m, 1H), 1.62-1.53 (m, 1H), 1.00-0.94 (m, 6H)

MS (ESI): m/z 272.84 [M+H]⁺

### Step 2

Compound 497 (55 mg, 0.20 mmol) and 5-(4-fluorophenyl)-isoxazole-3-carboxylic acid (45 mg, 0.22 mmol) were coupled according to the method described for the preparation of compound 71 to furnish product 496 (42 mg, 45.1% yield) as a white solid.

¹H NMR (400 MHz, CDCl₃): δ 7.85-7.77 (m, 2H), 7.21-7.16 (m, 3H), 6.99-6.95 (m, 1H), 6.88 (s, 1H), 6.03 (brs, 1H), 5.46 and 5.36 (dd, J=10.0 & 4.4 Hz, 1H), 5.07-4.89 (m, 2H), 3.43 and 3.16 (dd, J=14.4 & 11.2 Hz, 1H), 1.99-1.70 (m, 3H) and 1.10-0.81 (m, 6H).

MS (ESI): m/z 463.92 [M+2]⁺

### Example 426

### (3S,6S)-6-(2-chlorothiophen-3-yl)-4-(5-(4-fluorophenyl)-1,2,4-oxadiazole-3-carbonyl)-3-isobutylpiperazin-2-one (499)

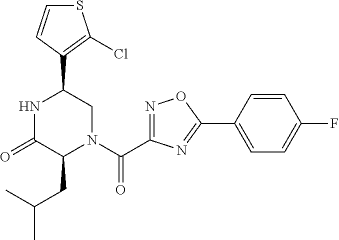

Compound 497 (55 mg, 0.20 mmol) and 66 (45 mg, 0.22 mmol) were coupled according to the method described for the preparation of compound 71 to furnish product 499 (50 mg, 53.6% yield) as a white solid.

¹H NMR (400 MHz, CDCl₃): δ 8.23-8.19 (m, 2H), 7.27-7.17 (m, 3H), 6.96 (dd, J=11.2 & 10.0 Hz, 1H), 6.10 (brs, 1H), 5.38 and 4.97 (m, 1H), 5.12 and 5.03 (dd, J=11.2 & 4.0 Hz, 1H), 4.94 and 4.35 (m, 1H), 3.42 and 3.20 (dd, J=14.0 & 11.2 Hz, 1H), 1.99-1.76 (m, 3H) and 1.11-0.76 (m, 6H).

MS (ESI): m/z 463.9 [M+H]⁺

### Example 427

### (3S,6S)-6-(2-chlorothiophen-3-yl)-4-((1R,2R)-2-(4-fluorophenyl)cyclopropane-carbonyl)-3-isobutylpiperazin-2-one (500)

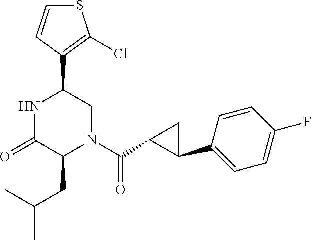

Compound 497 (55 mg, 0.20 mmol) and 60 (40 mg, 0.22 mmol) were coupled according to the method described for the preparation of compound 71 to furnish product 500 (30 mg, 34.2% yield) as a white solid.

¹H NMR (400 MHz, CDCl₃): δ 7.20-6.93 (m, 6H), 6.01 (brs, 1H), 5.28 and 4.92 (dd, J=8.8 & 4.0 Hz, 1H), 4.87-4.78 (m, 1H), 4.62 (dd, J=8.0 & 4.0 Hz, 0.5H), 4.20 (dd, J=14.4 & 4.4 Hz, 0.5H), 3.33 and 2.94 (dd, J=14.4 & 11.2 Hz, 1H), 2.60-2.54 (m, 1H), 2.02-1.65 (m, 5H), 1.36-1.24 (m, 1H) and 1.04-0.94 (m, 6H).

MS (ESI): m/z 435.9 [M+H]⁺

### Example 428

### Synthesis of (2R,5S)-4-(5-(4-fluorophenyl)isoxazole-3-carbonyl)-5-isobutyl-6-oxopiperazine-2-carboxylic acid (501)

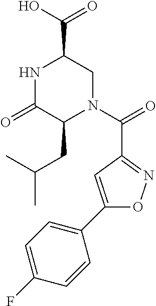

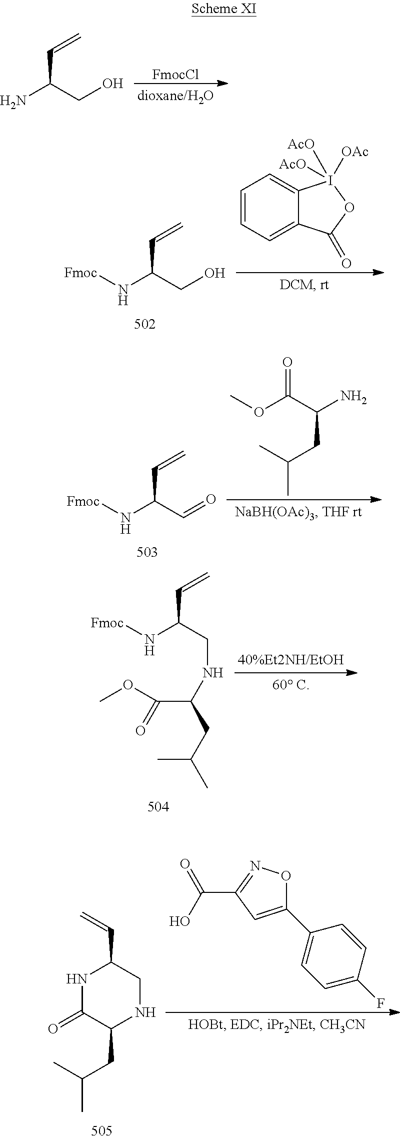

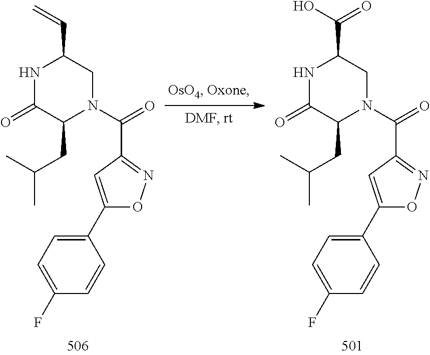

### Step 1: (S)-(9H-fluoren-9-yl)methyl (1-hydroxybut-3-en-2-yl)carbamate (502)

To (S)-2-Amino-but-3-en-1-ol (HCl salt, 1 g, 8.1 mmol) was added a solution of K₂CO₃ (3.4 g, 24.3 mmol) in water (50 mL) and a solution of FmocCl (2.3 g, 8.91 mmol) in dioxane (50 mL) in sequence at 0° C. After 3 h stirring at room temperature, the reaction mixture was extracted with dichloromethane (3×100 mL), washed with sat. aqueous NH₄Cl (50 mL) and brine (50 mL). The organic layer was dried over anhydrous Na₂SO₄. After filtration and concentration in vacuo, the residue was purified by column to give the product 502 (2.5 g, yield: 100%) as a white solid.

¹H NMR (400 MHz, CDCl3): δ 7.77 (2H, d, J=7.6 Hz), 7.60 (2H, d, J=7.6 Hz), 7.41 (2H, t, J=7.6 Hz), 7.32 (2H, t, J=7.6 Hz), 5.89-5.74 (1H, m), 5.25 (2H, d, J=12.4 Hz), 5.12 (1H, s), 4.45 (2H, d, J=6.8 Hz), 4.40-4.26 (1H, m), 4.22 (1H, t, J=6.8 Hz), 3.78-3.61 (2H, m), 1.93 (1H, s).

MS (ESI): m/z 310.0 [M+H]⁺.

### Step 2: (S)-(9H-fluoren-9-yl)methyl (1-oxobut-3-en-2-yl)carbamate (503)

To a solution of 502 (1.85 g, 6 mmol) in anhydrous dichloromethane (30 mL) was added Dess-Martin's reagent (5.33 g, 12.6 mmol) at room temperature. After 1 h stirring at room temperature, the reaction mixture was diluted with ether (21 mL), and a solution of sodium thiosulfate (10.43 g, 66 mmol) in sat. aq. NaHCO₃ (20 mL) was added. The mixture was stirred rapidly for 10 min until both phases were clear. Two layers were separated and the aqueous layer was extracted with ether (3×50 mL). The combined organic layers were washed sequentially with sat. aq. NaHCO₃ (10 ml), water (10 mL), and brine (10 mL), then dried over anhydrous Na₂SO₄ to furnish 503 (1.8 g, quantitative yield) as a white solid (1.8 g, yield: 100%) and was used directly for next step without purification.

MS (ESI): m/z 308.0 [M+1]⁺.

### Step 3: (S)-methyl 2-(((S)-2-((((9H-fluoren-9-yl)methoxy)carbonyl)amino)but-3-en-1-yl)amino)-4-methylpentanoate (504)

To a solution of 503 (1.842 g, 6 mmol) in THF (20 mL) was added (S)-2-Amino-4-methyl-pentanoic acid methyl ester (HCl salt, 1.2 g, 6.6 mmol), NaBH(OAc)₃ (1.9 g, 9 mmol) was added and stirred at room temperature overnight before it was quenched with sat. aqueous NaHCO₃ and extracted with EtOAc (3×100 mL). Organic layers were combined and dried over anhydrous Na₂SO₄. After filtration and concentration in vacuo, the residue was purified by column to give 504 (1.1 g, 42% yield) as pale brownish solid.

¹H NMR (400 MHz, CDCl3): δ 9.25 (1H, s), 7.78-7.75 (2H, m), 7.62-7.60 (2H, m), 7.41 (2H, t, J=7.6 Hz), 7.43-7.38 (2H, m), 7.34-7.29 (2H, m), 5.81-5.74 (1H, m), 5.24-5.17 (2H, m), 4.46-4.39 (3H, m), 4.27-4.21 (2H, m), 3.71 (3H, s), 3.30 (1H, t, J=7 Hz), 1.87-1.67 (3H, m), 0.98-0.87 (6H, m).

MS (ESI): m/z 437.3 [M+H]⁺.

### Step 4: (3S,6S)-3-isobutyl-6-vinylpiperazin-2-one (505)

To a solution of 504 (0.55 g, 1.26 mmol) in EtOH (6 ml) was added Et₂NH (4 mL) at room temperature. The solution was heated to 60° C. and stirred at the same temperature overnight. After cooled to room temperature and concentrated in vacuo, the residue was purified by column to give 505 (92.3 mg, 40.2% yield) as a colorless solid.

¹H NMR (400 MHz, CD3OD): δ 5.93-5.85 (1H, m), 5.26-5.21 (2H, m), 4.03-3.95 (1H, m), 3.34-3.29 (1H, m), 3.02 (1H, dd, J=4.8, 13.4 Hz), 2.85 (1H, dd, J=4.8, 13.4 Hz), 1.88-1.80 (1H, m), 1.72-1.64 (1H, m), 1.55-1.48 (1H, m), 0.95 (3H, d, J=6.4 Hz), 0.92 (3H, d, J=6.4 Hz).

MS (ESI): m/z 183.0 [M+H]⁺.

### Step 5: (3S,6S)-4-(5-(4-fluorophenyl)isoxazole-3-carbonyl)-3-isobutyl-6-vinylpiperazin-2-one (506)

A mixture of 505 (61.9 mg, 0.34 mmol) and 5-(4-Fluoro-phenyl)-isoxazole-3-carboxylic acid (84.4 mg, 0.41 mmol), HOBT hydrate (65.1 mg, 0.425 mmol), EDC (78.2 mg, 0.41 mmol), and di-isopropylethylamine (0.12 ml, 0.68 mmol) in CH₃CN (5 mL) were stirred at room temperature overnight. After removal of solvents, the residue was purified by column to give 506 (37.5 mg, 30% yield) as a brow solid.

¹H NMR (400 MHz, CDCl3): δ 7.80-7.77 (2H, m), 7.21-7.15 (2H, m), 6.87 (1H, s), 6.20 (1H, s), 5.76-5.68 (1H, m), 5.43 (1H, d, J=17.2 Hz), 5.32 (1H, d, J=10 Hz), 5.36-5.27 (1H, m), 4.88-4.82 (1H, m), 4.28-4.21 (1H, m), 3.21 (1H, dd, J=11, 14 Hz), 1.92-1.67 (3H, m), 1.07 (3H, d, J=6.4 Hz), 0.98 (3H, d, J=6.4 Hz).

MS (ESI): m/z 372.1 [M+H]⁺.

### Step 6: (2R,5S)-4-(5-(4-fluorophenyl)isoxazole-3-carbonyl)-5-isobutyl-6-oxopiperazine-2-carboxylic acid (501)

To a solution of 506 (92.9 mg, 0.25 mmol) in DMF (2 mL) was added OsO₄ (2.5% in t-BuOH, 0.03 mL, 0.0025 mmol). The reaction mixture was stirred at room temperature for 5 min before adding Oxone (614.8 mg, 1 mmol) in one portion. After 3 h stirring at room temperature, Na₂SO₃ (189.06 mg, 1.5 mmol) was added to the reaction mixture and stirred for additional hour. EtOAc (20 mL) was then added to extract the product and 1N HCl (20 mL) was used to dissolve the salts. The organic extract was washed with 1NHCl (2×10 mL) and brine, dried over Na₂SO₄. After filtration and concentration in vacuo, the residue was purified by column to give 501 (58.4 mg, 60% yield) as a brow solid.

¹H NMR (400 MHz, CD3OD): δ 7.95-7.91 (2H, m), 7.30-7.25 (2H, m), 7.05 (1H, s), 5.16 (1H, dd, J=4, 10 Hz), 4.78-4.70 (1H, m), 4.18-4.10 (1H, m), 3.49-3.43 (1H, m), 1.89-1.68 (3H, m), 1.06 (3H, d, J=6 Hz), 0.99 (3H, d, J=6 Hz).

MS (ESI): m/z 390.0 [M+H]⁺.

### Example 429

### (2R,5S)-4-(5-(4-fluorophenyl)isoxazole-3-carbonyl)-5-isobutyl-N,N-dimethyl-6-oxopiperazine-2-carboxamide(507)

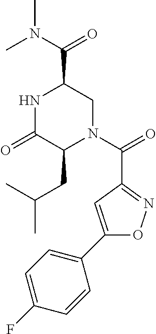

According to the method described for compound 89, Compound 501 (37.4 mg, 0.1 mmol) and dimethylamine (2M in THF, 0.06 mL, 0.115 mmol) were coupled to give the product 507 (10.4 mg, 25% yield) as a white solid.

¹H NMR (400 MHz, CD3OD): δ 7.98-7.92 (2H, m), 7.31-7.26 (2H, m), 7.10 (1H, s), 5.18-5.15 (1H, m), 4.97-4.89 (1H, m), 4.79-4.73 (1H, m), 3.44 (1H, dd, J=11.2, 14.2 Hz), 3.19 (3H, s), 2.97 (3H, s), 1.96-1.66 (3H, m), 1.08 (3H, d, J=6.4 Hz), 0.99 (3H, d, J=6.4 Hz).

MS (ESI): m/z 417.1 [M+H]⁺.

### Example 430

### (2R,5S)-4-(5-(4-fluorophenyl)isoxazole-3-carbonyl)-5-isobutyl-N-methyl-6-oxopiperazine-2-carboxamide(508)

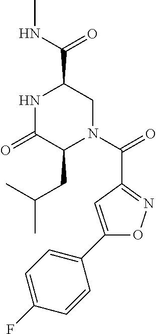

According to the method described for compound 89, Compound 501 (37.4 mg, 0.1 mmol) and methylamine (HCl salt, 8 mg, 0.12 mmol) were coupled to give the product 508 (17.6 mg, 44% yield) as a white solid.

¹H NMR (400 MHz, CD3OD): δ 7.96-7.93 (2H, m), 7.31-7.26 (2H, m), 7.07 (1H, s), 5.19-5.16 (1H, m), 4.72-4.67 (1H, m), 4.28-4.23 (1H, m), 3.55 (1H, dd, J=11.2, 14.2 Hz), 2.75 (3H, s), 1.98-1.66 (3H, m), 1.06 (3H, d, J=6.4 Hz), 1.00 (3H, d, J=6.4 Hz).

MS (ESI): m/z 403.1 [M+H]⁺.

### Example 431

### (2R,5S)—N-(2,2-dimethoxyethyl)-4-(5-(4-fluorophenyl)isoxazole-3-carbonyl)-5-isobutyl-6-oxopiperazine-2-carboxamide(509)

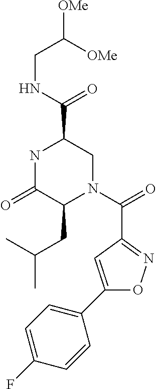

According to the method described for compound 89, Compound 501 (96 mg, 0.25 mmol) and 2,2-Dimethoxy-ethylamine (0.03 ml, 0.3 mmol) were coupled (96 mg, 0.25 mmol) to give the product 509 (60 mg, 50% yield) as pale yellow gum.

¹H NMR (400 MHz, CDCl3): δ 7.81-7.77 (2H, m), 7.22-7.17 (2H, m), 6.91 (1H, s), 6.60 (1H, s), 6.32 (1H, t, J=5.6 Hz), 5.29-5.26 (1H, m), 5.11 (1H, dd, J=4, 14 Hz), 4.43-4.38 (3H, m), 3.50-3.36 (8H, m), 1.93-1.65 (3H, m), 1.08 (3H, d, J=6.4 Hz), 0.98 (3H, d, J=6.4 Hz).

MS (ESI): m/z 477.1 [M+H]⁺.

### Example 432

### Synthesis of (3S,6R)-4-(5-(4-fluorophenyl)isoxazole-3-carbonyl)-3-isobutyl-6-(oxazol-2-yl)piperazin-2-one (510)

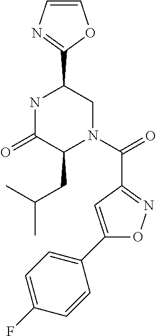

To a solution of 509 (11.2 mg, 0.0235 mmol) in DCM (2 mL) was added TFA (0.6 mL) at room temperature. After 5 h stirring at room temperature, the reaction mixture was concentrated in vacuo. The residue was purified by column to give 510 (7.3 mg, 75% yield) as colorless solid.

¹H NMR (400 MHz, CDCl3): δ 7.80-7.77 (2H, m), 7.21-7.17 (2H, m), 6.92 (1H, s), 6.83 (1H, s), 6.69 (1H, d, J=5.6 Hz), 5.92 (1H, d, J=5.6 Hz), 5.42-5.38 (1H, m), 5.34-5.28 (1H, m), 4.51 (1H, dd, J=4.6, 11 Hz), 3.73 (1H, dd, J=11, 15 Hz), 1.91-1.68 (3H, m), 1.09 (3H, d, J=6.4 Hz), 1.00 (3H, d, J=6.4 Hz).

MS (ESI): m/z 413.0 [M+H]⁺.

### Example 433

### Synthesis of (3S,6S)-4-(1-(5-(4-fluorophenyl)isoxazol-3-yl)ethyl)-3-isobutyl-6-phenylpiperazin-2-one(511)

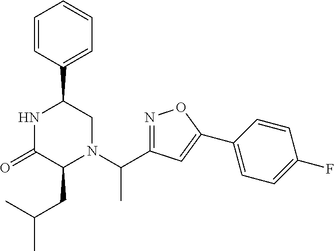

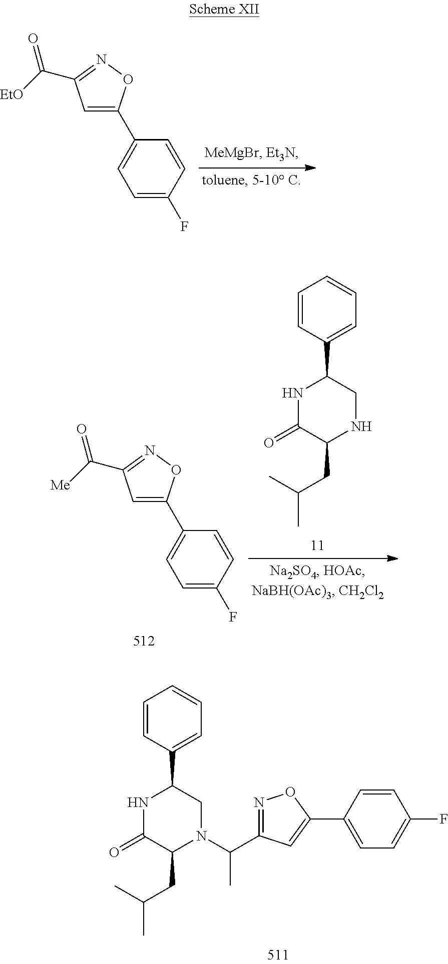

### Step 1. 1-(5-(4-fluorophenyl)isoxazol-3-yl)ethanone(512)

To a solution of ethyl 5-(4-fluorophenyl)isoxazole-3-carboxylate (1.95 g, 8.29 mmol) and Et₃N (6.9 mL, 49.7 mmol) in toluene (60 mL), MeMgBr (1.4M, in THF/Tol., 13 mL, 18.25 mmol) was added dropwise at 5-10° C. After 2 h stirring at 5-10° C., 2N aqueous HCl (22.8 mL) was added. The mixture was washed with water, sat. aqueous NaHCO₃, and dried over Na₂SO4. After filtration and concentration in vacuo, the residue was purified by column to give 512 (1.223 g, 72% yield) as a white solid.

¹H NMR (400 MHz, CDCl3): δ 7.81-7.78 (2H, m), 7.21-7.15 (2H, m), 6.83 (1H, s), 2.69 (3H, s).

### Step 2: (3S,6S)-4-(1-(5-(4-fluorophenyl)isoxazol-3-yl)ethyl)-3-isobutyl-6-phenyl-pipera-zin-2-one (511)

A mixture of 11 (226.7 mg, 0.98 mmol), 1 (100 mg, 0.49 mmol), anhydrous Na₂SO₄ (159 mg, 1.12 mmol) and acetic acid (0.028 mL, 0.49 mmol) in dichloromethane (10 mL) were stirred at room temperature for 1 h followed by addition of triacetoxy sodiumborohydride (623.1 mg, 2.94 mmol). The reaction mixture was continued to stir at room temperature overnight and quenched with water. The mixture was extracted with dichloromethane (3×15 mL). The organic layers were combined, dried over Na₂SO₄, and filtered. After concentration in vacuo, the residue was purified by column to give 511 (24.8 mg, 12% yield) as a colorless gum.

¹H NMR (400 MHz, CDCl3): δ 7.78 (2H, dd, J=4.4, 8.8 Hz), 7.35-7.21 (5H, m), 7.16 (2H, t, J=8.8 Hz), 6.47 (1H, s), 5.79 (1H, s), 4.90 (1H, dd, J=4.6, 10.8 Hz), 4.36 (1H, q, J=6.4 Hz), 3.63 (1H, dd, J=4.6, 9.2 Hz), 2.93 (1H, dd, J=10.8, 14.8 Hz), 2.78 (1H, dd, J=4.8, 14.6 Hz), 1.96-1.69 (3H, m), 1.51 (3H, d, J=6.4 Hz), 1.00-0.98 (6H, m).

MS (ESI): m/z 422.1 [M+H]⁺.

### (3S,6R)-6-(tert-butoxymethyl)-3-isobutylpiperazin-2-one (513)

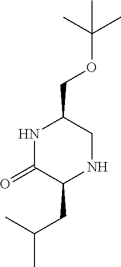

Synthesized from FMOC-L-Ser-O^{t}Bu (42.0 g, 0.114 mol) and Hydrochloride salt of L-Leucine methyl ester (21.0 g, 0.115 mol) by the method described for the compound 7 (Scheme II) to afford the product 513 (4.2 g, overall yield: 5.7%) as a colorless gum.

¹H NMR (400 MHz, DMSO): δ 7.370-7.375 (m, 1H), 3.35-3.39 (m, 1H), 3.21-3.326 (m, 2H,), 3.03-3.06 (m, 1H), 2.77-2.84 (m, 2H), 1.72-174 (m, 1H), 1.566-1.576 (m, 1H), 1.345-1.347 (m, 1H), 1.121-1.126 (m, 9H) and 0.822-0.881 (m, 6H).

MS (ESI): m/z 243.2 [M+H]⁺

### Example 434

### (2S,5R)-tert-butyl 2-isobutyl-5-(oxazol-5-yl)-3-oxopiperazine-1-carboxylate (514)

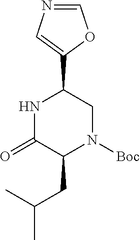

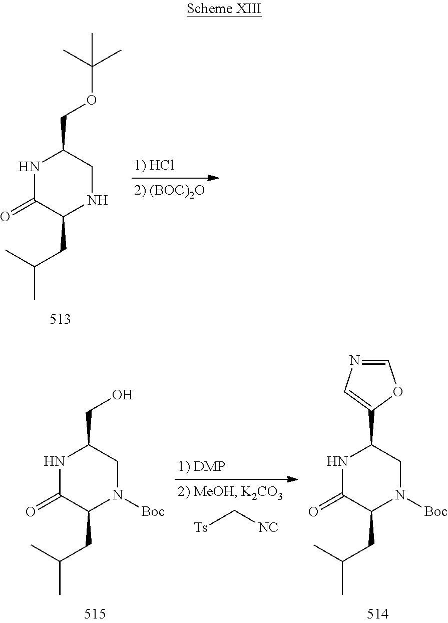

### Step 1: (2S,5R)-tert-butyl 5-(hydroxymethyl)-2-isobutyl-3-oxopiperazine-1-carboxylate (515)

To the compound 513 (2.00 g, 8.25 mmol) was added HCl in dioxane (4 N, 20 ml) and it was stirred at 70° C. for 1.5 h. LCMS showed the product with no starting material left. It was concentrated to give the crude ^{t}Bu deprotected compound and this crude compound dissolved in DMF (20 ml) and DCM (10 ml), was added DIEA (3.5 ml) and Boc-anhydride (1.60 ml, 6.96 mmol) in DCM (5 ml). After 5 h stirring, reaction micture was concentrated to ca 5 ml, and EtOAc (15 ml) and water (15 ml) were added. The organic layer was separated and washed with brine, dried (MgSO4), concentrated and purified by silica column eluted with 0 to 90% EtOAc in hexanes to give the product 515 (2.06 g, 87% in two steps) as a white solid.

¹H NMR (DMSO-d₆) δ 7.69 (s, 1H), 4.88 (t, J=5.6 Hz, 1H), 4.40-4.22 (m, 1H), 4.11-3.90 (m, 1H), 3.44-3.24 (m, 2H), 2.90-2.76 (m, 2H), 1.65-1.56 (m, 2H), 1.51-1.46 (m, 1H), 1.41 (s, 9H), 0.91 (m, 6H).

MS (ESI): m/z 287.2 [M+H]⁺

### Step 2: (2S,5R)-tert-butyl 2-isobutyl-5-(oxazol-5-yl)-3-oxopiperazine-1-carboxylate (514)

To a solution of the alcohol 515 (0.50 g, 1.75 mmol) in DCM (25 ml) at 0° C. was added Dess-Martin reagent (0.965 g, 4.55 mmol) and water (0.5 ml). It was warmed to rt and stirred for 6 h. Then, iPrOH (0.3 ml) was added and the mixture was stirred for 15 min. Sat. aq sodium thiosulfate (10 ml) and sat. aq NaHCO3 (10 ml) were added. Layers were separated and the aq layer was extracted with EtOAc (2×5 ml). Combined organics were washed with brine, dried (MgSO4), and concentrated to give the crude aldehyde product.

To the crude aldehyde in MeOH (20 ml) was added tosylmethyl isocyanide (480 mg, 2.46 mmol) and K₂CO₃ (483 mg, 3.50 mmol). It was warmed to 70° C. for 1 h. Then, the reaction was concentrated. EtOAc (30 ml) and water (20 ml) were added. Layers were separated and the aq layer was extracted with EtOAc (20×2 ml). Combined organics were washed with brine, dried (MgSO4), and concentrated, and purified by silica column eluted with 0 to 95% EtOAc in hexanes to give the product 514 (78 mg, 14% yield in two steps) as a white solid.

¹H NMR (CDCl₃) δ 7.90 (s, 1H), 7.08 (s, 1H), 5.93 (bs, 1H), 4.88-4.84 (m, 1H), 4.69-4.64 (m, 1H), 4.50-4.44 (m, 1H), 3.18 (t, J=12.4 Hz, 1H), 1.78-1.62 (m, 3H), 1.02 (d, J=5.6 Hz, 3H), 0.97 (d, J=6.0 Hz, 3H)

MS (ESI): m/z 324.1 [M+H]⁺

### Example 435

### Synthesis of (3S,6R)-4-(5-(4-fluorophenyl)isoxazole-3-carbonyl)-3-isobutyl-6-(oxazol-5-yl)piperazin-2-one (516)

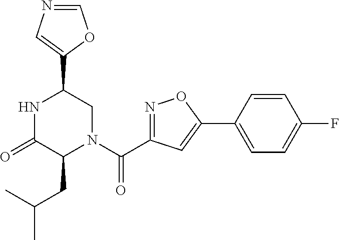

To a solution 514 (26 mg, 0.078 mmol) in DCM (1.5 ml) at 0° C. was added 4 N HCl in dioxane (0.5 ml). It was warmed to rt and stirred for 1 h and then, concentrated to give the crude Boc deprotected amine HCl salt compound. To a stirred solution of 5-(4-Fluoro-phenyl)-isoxazole-3-carboxylic acid (16 mg, 0.078 mmol) and HATU (22.9 mg, 0.079 mmol) in DMF (1.5 ml), crude amine salt in DMF (0.5 ml) and DIEA (0.1 ml) was added and stirred for 1 h. Reactiom mixture was concentrated and the crude product was on column chromatography (eluted with 0 to 95% EtOAc in hexanes) gave the product 516 (24.6 mg, 71% yield) as a white solid.

¹H NMR (CDCl₃) δ 8.01 (s, 1H), 7.80-7.77 (m, 2H), 7.21-7.16 (m, 2H), 7.02 (bs, 1H), 6.90 (s, 1H), 6.86 (s, 0.6H), 6.83 (s, 0.4H), 5.52-5.44 (m, 0.4H), 5.37 (m, 0.6H), 5.18-4.88 (m, 2H), 3.65 (m, 0.6H), 3.38 (m, 0.4H), 1.93-1.86 (m, 1H), 1.76-1.71 (m, 1H), 1.10-0.80 (m, 6H).

MS (ESI): m/z 413.2 [M+H]⁺

### Example 436

### Synthesis of (3S,6R)-3-isobutyl-6-(oxazol-5-yl)-4-((1R,2R)-2-phenylcyclopropane-carbonyl)piperazin-2-one (517)

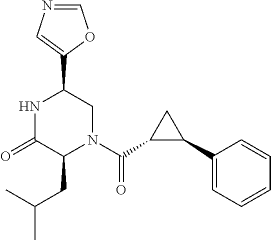

Compound 514 (26 mg, 0.078 mmol) and trans (1R,2R)-2-Phenyl-cyclopropanecarboxylic acid 54 (13 mg, 0.079 mmol) were coupled according to the method described for the preparation of compound 516 to furnish product 517 (16 mg, 55% yield) as a white solid.

¹H NMR (CDCl₃) δ 7.99 (s, 1H), 7.31-7.07 (m, 5H), 6.70 (s, 1H), 5.87 (d, J=6.4 Hz, 1H), 5.33 (dd, J=4.0, 10.4 Hz, 0.5H), 5.08 (dd, J=4.4, 11.2 Hz, 0.5H) 4.98-4.87 (m, 1H), 4.70-4.67 (m, 0.5H), 4.30 (dd, J=4.0, 13.2 Hz, 0.5H), 3.33 (dd, J=6.8, 14.4 Hz, 0.5H), 3.00 (dd, J=6.8, 14.4 Hz, 0.5H), 2.59 (m, 1H), 2.07 (m, 1H), 2.00-1.82 (m, 3H), 1.76-1.68 (m, 1H), 1.37 (m, 1H), 1.05-0.95 (m, 6H).

MS (ESI): m/z 368.2 [M+H]⁺

### Example 437

### Synthesis of (3S,6R)-4-(5-(4-fluorophenyl)-1,2,4-oxadiazole-3-carbonyl)-3-isobutyl-6-(oxazol-5-yl)piperazin-2-one (518)

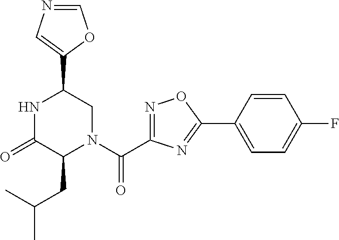

Compound 514 (26 mg, 0.078 mmol) and 66 (16 mg, 0.079 mmol) were coupled according to the method described for the preparation of compound 516 to furnish product 518 (19 mg, 59% yield) as a white solid.

¹H NMR (DMSO-d₆) δ 8.64 (s, 0.3H), 8.60 (s, 0.7H), 8.45 (s, 0.3H), 8.39 (s, 0.7H), 8.27-8.22 (m, 2H), 7.56-7.50 (m, 2H), 7.30 (s, 0.3H), 7.26 (s 0.7H), 4.98-4.93 (m, 2H), 4.32 (m, 1H), 3.64 (dd, J=11.6, 14.4 Hz, 1H), 1.96-1.88 (m, 1H), 1.78-1.61 (m, 2H), 1.01 (d, J=6.4 Hz, 2.5H), 0.97 (d, J=6.8 Hz, 2.5H), 0.87-0.65 (m, 1H).

MS (ESI): m/z 414.2 [M+H]⁺

### Example 438

### Synthesis of (3S,6R)-4-(3-(4-fluorophenyl)isoxazole-5-carbonyl)-3-isobutyl-6-(oxazol-5-yl)piperazin-2-one (519)

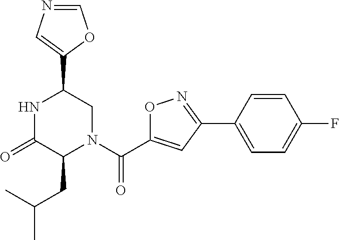

Compound 514 (26 mg, 0.078 mmol) and 3-(4-fluorophenyl)isoxazole-5-carboxylic acid (16 mg, 0.079 mmol) were coupled according to the method described for the preparation of compound 516 to furnish product 519 (22 mg, 68% yield) as a white solid.

¹H NMR (DMSO-d₆) δ 8.62 (s, 0.3H), 8.59 (s, 0.7H), 8.44 (s, 0.3H), 8.40 (s, 0.7H), 8.03-8.00 (m, 2H), 7.72 (s, 0.3H), 7.70 (s, 0.7H), 7.40 (t, J=8.8 Hz, 2H), 7.30 (s, 0.3H), 7.24 (s, 0.7H), 5.08 (m, 1H), 4.93 (m, 1H), 4.30 (m, 1H), 3.69 (dd, J=11.2, 14.4 Hz, 1H), 1.93-1.86 (m, 1H), 1.76-1.60 (m, 2H), 0.98 (d, J=6.4 Hz, 2.5H), 0.95 (d, J=6.4 Hz, 2.5H), 0.88-0.66 (m, 1H).

MS (ESI): m/z 413.2 [M+H]⁺

### Example 439

### Synthesis of (3S,6R)-4-((E)-3-(2,4-difluorophenyl)acryloyl)-3-isobutyl-6-(oxazol-5-yl)piperazin-2-one(520)

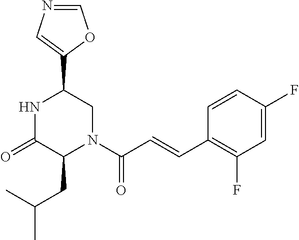

Compound 514 (26 mg, 0.078 mmol) and (E)-3-(2,4-difluorophenyl)acrylic acid (15 mg, 0.079 mmol) were coupled according to the method described for the preparation of compound 516 to furnish product 520 (27 mg, 88% yield) as a white solid.

¹H NMR (DMSO-d₆) δ 8.52 (s, 0.4H), 8.43 (s, 0.6H), 8.41 (s, 1H), 8.11 (m, 1H), 7.67-7.62 (m, 1H), 7.44-7.18 (m, 4H), 5.01 (m, 1H), 4.87 (m, 1H), 4.61 (m, 1H), 3.53 (m, 1H), 1.84-1.78 (m, 1H), 1.64-1.52 (m, 2H), 0.96-0.82 (m, 6H).

MS (ESI): m/z 390.2 [M+H]⁺

### Example 440

### Synthesis of (3S,6R)-4-((1R,2R)-2-(4-fluorophenyl)cyclopropanecarbonyl)-3-isobutyl-6-(oxazol-5-yl)piperazin-2-one (521)

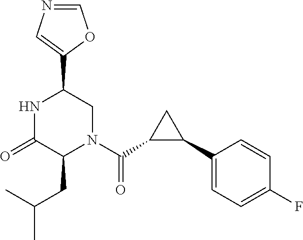

Compound 514 (26 mg, 0.078 mmol) and 60 (14 mg, 0.079 mmol) were coupled according to the method described for the preparation of compound 516 to furnish product 521 (23 mg, 76% yield) as a white solid.

¹H NMR (DMSO-d₆) δ 8.45-8.38 (m, 2H), 7.25-7.22 (m, 3H), 7.13-7.06 (m, 2H), 4.89 (m, 2H), 4.78-4.50 (m, 2H), 3.52 (dd, J=11.2, 14.8 Hz, 1H), 1.80-1.71 (m, 1H), 1.62-1.50 (m, 2H), 1.46-1.36 (m, 1H), 1.30-1.18 (m, 2H), 0.96-0.88 (m, 6H).

MS (ESI): m/z 386.2 [M+H]⁺

### Example 441

### Synthesis of (3S,6R)-4-(5-(2,4-difluorophenyl)isoxazole-3-carbonyl)-3-isobutyl-6-(oxazol-5-yl)piperazin-2-one (522)

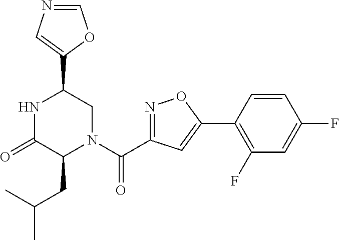

Compound 514 (26 mg, 0.078 mmol) and 5-(2,4-difluorophenyl)isoxazole-3-carboxylic acid (14 mg, 0.079 mmol) were coupled according to the method described for the preparation of compound 516 to furnish product 522 (24 mg, 71% yield) as a white solid.

¹H NMR (DMSO-d₆) δ 8.58 (s, 1H), 8.44 (s, 0.3H), 8.39 (s, 0.7H), 8.07 (m, 1H), 7.60 (m, 1H), 7.35 (m, 1H), 7.30 (s, 0.3H), 7.23 (s, 0.7H), 7.21 (d, J=2.8 Hz, 1H), 5.02-4.92 (m, 2H), 4.68 (m, 1H), 4.40 (m, 1H), 3.65 (dd, J=11.2, 14.4 Hz, 1H), 1.95-1.86 (m, 1H), 1.76-1.60 (m, 2H), 1.00 (d, J=6.0 Hz, 2.5H), 0.96 (d, J=6.4 Hz, 2.5H), 0.87-0.65 (m, 1H).

MS (ESI): m/z 431.2 [M+H]⁺

### Example 442

### Synthesis of (3S,6R)-4-(5-(4-fluorophenyl)isoxazole-3-carbonyl)-3-isobutyl-6-(1H-pyrrol-2-yl)piperazin-2-one (523) and (3S,6S)-4-(5-(4-fluorophenyl)isoxazole-3-carbonyl)-3-isobutyl-6-(1H-pyrrol-2-yl)piperazin-2-one (524)

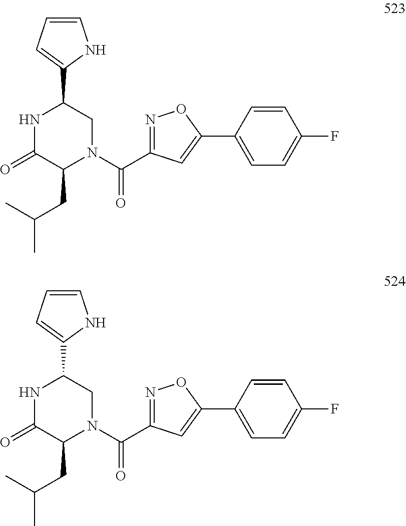

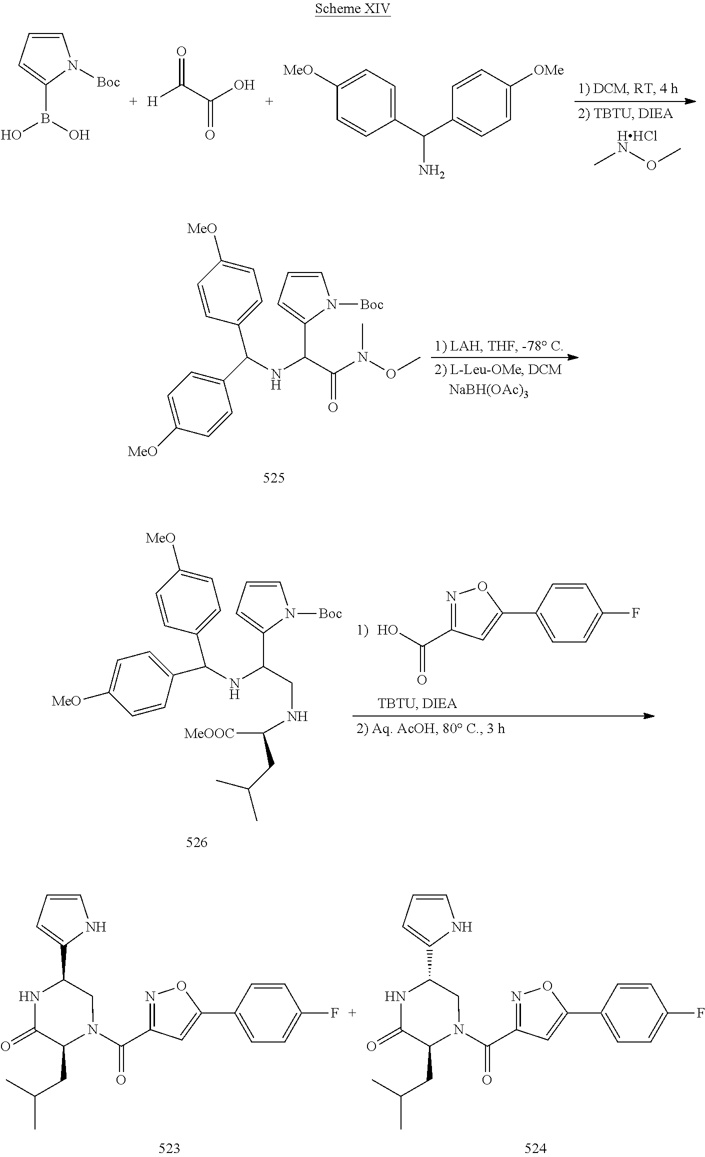

### Step 1: Synthesis of tert-butyl 2-(1-((bis(4-methoxyphenyl)methyl)amino)-2-(methoxy-(methyl)-amino)-2-oxoethyl)-1H-pyrrole-1-carboxylate (525)

To a stirred solution of glyoxylic acid monohydrate (0.92 g, 10.11 mmol) in CH₂Cl₂ (70 mL) was added bis(4-methoxyphenyl)methanamine (2.46 g, 10.11 mmol), followed by (1-(tert-butoxycarbonyl)-1H-pyrrol-2-yl)boronic acid (2.13 g, 10.11 mmol) at once. The reaction mixture was stirred at room temperature for 2 minutes to form clear solution. The solution was purged with Ar (2 min) and sealed reaction mixture was stirred for over night. The solvent was removed under reduced pressure to dryness to yield crude acid product (4.5 g, 95%) as pale yellow foam. Crude acid (4.5 g, 10.07 mmol), TBTU (4.87 g, 15.11 mmol), dimethylhydroxylamine hydrochloride (1.47 g, 15.11 mmol) were combined and added dry CH₃CN (50 mL). This mixture was stirred at room temperature and added DIEA (5.3 mL, 30.22 mmol) and continued stirring for 12 h. The solvent was concentrated under reduced pressure and the crude product was purified by column chromatography using EtOAc/Hexane (5 to 40%) to afford compound 525 (5.1 g, 98% yield) as pale yellow foam.

¹H NMR (400 MHz, CDCl₃): δ 7.36-7.25 (m, 4H), 7.15-7.14 (m, 1H), 6.82-6.76 (m, 4H), 6.18 (s, 1H), 6.07 (t, J=3.2 Hz, 1H), 5.72 (brs, 1H), 4.82 (s, 1H) 3.76 (s, 3H), 3.74 (s, 3H) 3.24 (s, 3H), 3.13 (s, 3H). 2.58 (brs, 1H), 1.53 (s, 9H).

MS (ESI): m/z 510.1 [M+H]⁺

### Step 2: Synthesis of tert-butyl 2-(1-((bis(4-methoxyphenyl)methyl)amino)-2-(((S)-1-methoxy-4-methyl-1-oxopentan-2-yl)amino)ethyl)-1H-pyrrole-1-carboxylate (526)

To a solution of compound 525 (1.5 g, 2.94 mmol) in dry THF (30 mL) was cooled at −78° C. and added LiAlH₄ (220 mg, 5.89 mmol) at once. The reaction was stirred for 3 h at same temp under Ar atmosphere. The reaction was quenched with sat NH₄Cl solution by drop wise addition (at this time solution was warmed to 0-25° C.). This mixture was partitioned between EtOAc/Brine, and extracted with EtOAc. The EtOAc layers were combined and washed with brine, and dried (MgSO₄). The crude aldehyde (unstable, slowly decomposes at room temperature) was dried and used for the next step. To a mixture of aldehyde compound (01.2 g, 2.66 mmol) and L-Leu-OMe-HCl (0.58 g, 2.66 mmol) in dichloromethane (24 mL), NaBH(OAc)₃ (790 mg, 3.73 mmol) was added and stirred at room temperature under Ar atmosphere over 6 h. The reaction mixture was quenched with saturated aqueous NaHCO₃ solution (25 mL) and the aqueous solution was extracted with EtOAc (30 mL) and dried (Na₂SO₄). The solvents were removed under vacuum and the crude product was purified by column chromatography using EtOAc/Hex (0 to 40%) to afford compound 526 (1.54 g, 67% yield) as a pale yellow foam.

¹H NMR (400 MHz, CDCl₃): δ 7.24-7.21 (m, 4H), 6.80-6.78 (m, 5H), 6.15-6.14 (m, 2H), 4.72 (s, 1H), 4.30 (brs, 1H), 3.79-3.75 (m, 4H), 3.64 (s, 3H) 3.26-3.90 (m, 2H), 2.68-2.65 (m, 1H) 2.05 (brs, 1H), 1.70-1.65 (m, 1H), 1.48-1.41 (m, 11H), 0.91-0.85 (m, 6H).

MS (ESI): m/z 580.1 [M+H]⁺

### Step 3: Synthesis of (3S,6R)-4-(5-(4-fluorophenyl)isoxazole-3-carbonyl)-3-isobutyl-6-(1H-pyrrol-2-yl)piperazin-2-one (523) and (3S,6S)-4-(5-(4-fluorophenyl)isoxazole-3-carbonyl)-3-isobutyl-6-(1H-pyrrol-2-yl)piperazin-2-one (524)

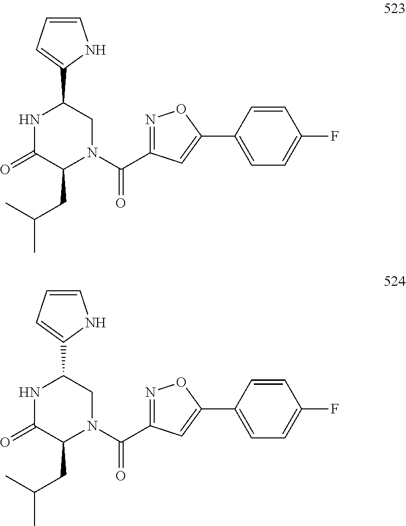

To a stirred solution of compound 526 (225 mg, 0.39 mmol), TBTU (161 mg, 0.5 mmol) and 5-(4-Fluoro-phenyl)-isoxazole-3-carboxylic acid (116 mg, 0.56 mmol) in anhydrous CH₃CN (6 mL), DIPE (0.41 mL, 2.33 mmol) was added and continued stirring for 24 h. The solvent was removed under reduced pressure and the crude product was purified by column chromatography using EtOAc/Hex (0 to 50%) to afford crude amide compound (250 mg, 83% yield) as pale yellow foam. The crude amide compound (250 mg, 0.32 mmol) was dissolved in 70% aqueous AcOH (6 mL) and heated under reflux (80° C.) over 24 h. The solvent was removed under reduced pressure and the crude product was purified by column chromatography (0 to 4% MeOH in CH₂Cl₂) to afford 523 (13 mg, 15% yield) and 524 (13 mg, 15% yield) as brown solids.

### Compound 523

¹H NMR (400 MHz, CDCl₃): δ 9.23-9.09 (d, J=53 Hz, 1H), 7.81-7.76 (m, 2H), 7.21-7.16 (m, 2H), 6.94-6.79 (m, 3H), 6.18-6.16 (m, 1H), 5.43-5.29 (m, 1H), 4.97-4.85 (m, 1H), 3.77 (brs, 1H), 3.51-3.16 (m, 1H), 1.90-1.84 (m, 2H), 1.74-1.60 (m, 1H), 1.09-0.77 (m, 6H).

MS (ESI): m/z 411.1 [M+H]⁺

### Compound 524

¹H NMR (400 MHz, CDCl₃): δ 9.05-8.81 (m, 1H), 7.79-7.65 (m, 2H), 7.19-7.08 (m, 2H), 7.08-6.61 (m, 3H), 6.05-5.91 (m, 1H), 5.45-5.29 (m, 1H), 4.83-4.58 (m, 1H), 3.93-3.48 (m, 2H), 1.96-1.81 (m, 2H), 1.78-1.67 (m, 1H), 1.04-0.75 (m, 6H).

MS (ESI): m/z 411.1 [M+H]⁺

### Example 443

### Synthesis of (5S)-5-isobutyl-2-phenylimidazolidin-4-one (527)

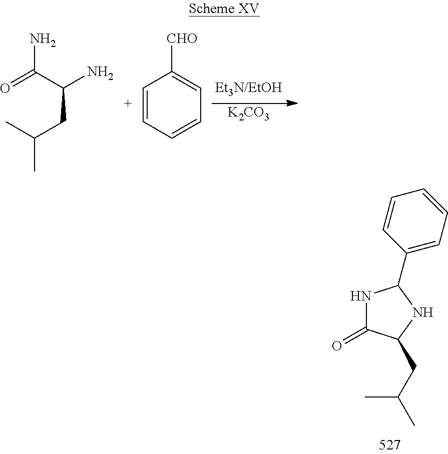

A mixture of L-leucinamide (1 g, 7.68 mmol), benzaldehyde (0.78 mL, 7.68 mmol), Et₃N (1.08 mL, 7.68 mmol), and K₂CO₃ (1.06 g, 7.68 mmol) in EtOH (8 mL) was heated at 60° C. over 24 h. The solvent was removed under reduced pressure and the residue was taken up in ether and filtered. The combined filtrate was concentrated and the crude product was on column chromatography (0 to 25% EtOAc in hexanes) gave cis and trans mixture 527 (1.13 g, 67% yield) as a white solid.

¹H NMR (400 MHz, CDCl₃): δ 7.44-7.24 (m, 6H), 5.56 (d, J=21.2 Hz, 1H), 3.64-3.52 (m, 1H), 1.92 (bs, 1H), 7.87-1.79 (m, 1H), 1.78-1.59 (m, 1H), 1.52-1.35 (m, 1H), 0.98-0.89 (m, 6H).

MS (ESI): m/z 219.0 [M+H]⁺

### Example 444

### (2R,5S)-1-(5-(4-fluorophenyl)isoxazole-3-carbonyl)-5-isobutyl-2-phenylimidazolidin-4-one (528) and (2S,5S)-1-(5-(4-fluorophenyl)isoxazole-3-carbonyl)-5-isobutyl-2-phenylimidazolidin-4-one (529)

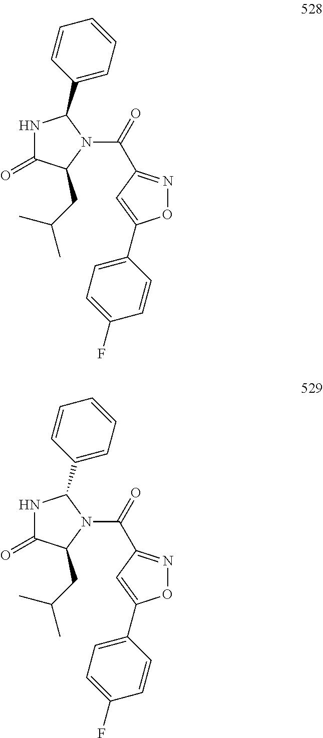

To a solution of Compound 527 (150 mg, 0.69 mmol), TBTU (330 mg, 1.03 mmol) and 5-(4-fluorophenyl)isoxazole-3-carboxylic acid (170 mg, 0.82 mmol) in anhydrous CH₃CN (5 mL), DIPEA (0.36 mL, 2.06 mmol) was added and stirred at 60° C. for 24 h. The solvents were removed under vacuum and the crude product was on column chromatography (0 to 40% EtOAc/in hexanes) afforded a mixture of cis and trans isomers. HPLC purification of the mixture furnished 528 (66 mg, 35.5% yield) and 529 (63 mg, 33.8% yield) as a white solids.

### Compound 528

¹H NMR (400 MHz, CDCl₃): δ 7.78-7.75 (m, 1H), 7.61-7.58 (m, 1H), 7.47-7.34 (m, 2H), 7.19-7.10 (m, 5H), 6.75-6.64 (m, 1H), 6.27-6.13 (m, 1H), 5.28-4.81 (m, 1H), 2.15-1.64 (m, 3H), 1.03-0.80 (m, 6H).

MS (ESI): m/z 408.0 [M+1]⁺

### Compound 529

¹H NMR (400 MHz, CDCl₃): δ 7.80-7.59 (m, 3H), 7.44-7.30 (m, 3H), 7.20-7.07 (m, 2H), 6.87-6.83 (m, 1H), 6.70 (brs, 1H), 6.59 (s, 1H), 5.15-4.72 (m, 1H), 2.19-2.13 (m, 1H), 1.89-1.68 (m, 2H), 1.07-0.86 (m, 6H).

MS (ESI): m/z 408.0 [M+1]⁺

### Example 445

### (2R,5S)-5-isobutyl-2-phenyl-1-((1S,2S)-2-phenylcyclopropanecarbonyl)imidazolidin-4-one (530) and (2R,5S)-5-isobutyl-2-phenyl-1-((1S,2S)-2-phenylcyclopropanecarbonyl)imidazolidin-4-one (531)

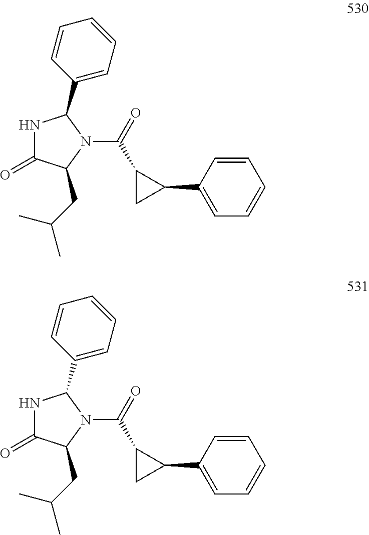

Compound 527 (100 mg, 0.46 mmol) and 54 (70 mg, 0.46 mmol) were coupled according to the procedure described for compound 528/529 to afford 530 (30 mg, 18% yield) and 531 (29 mg, 18% yield) as an oil.

### Compound 530

¹H NMR (400 MHz, CDCl₃): δ 7.47-7.06 (m, 10H), 6.66 (bs, 1H), 6.39 (bs, 1H), 4.54-4.26 (m, 1H), 2.54-2.47 (m, 1H), 2.19-2.05 (m, 1H), 1.93-1.58 (m, 4H), 1.35-1.16 (m, 1H), 1.04-0.85 (m, 6H).

MS (ESI): m/z 363.1 [M+H]⁺

### Compound 531

¹H NMR (400 MHz, CDCl₃): δ 7.46-7.14 (m, 8H), 7.09-6.97 (m, 3H), 6.33-6.14 (m, 1H), 4.57-4.32 (m, 1H), 2.44-2.39 (m, 1H), 2.07-2.02 (m, 1H), 1.79-1.43 (m, 4H), 1.37-1.25 (m, 1H), 1.09-0.67 (m, 6H).

MS (ESI): m/z 363.1 [M+H]⁺

### Example 446

### (2R,5S)-1-((1S,2S)-2-(4-fluorophenyl)cyclopropanecarbonyl)-5-isobutyl-2-phenyl-imidazolidin-4-one (532) and (2R,5S)-1-((1S,2S)-2-(4-fluorophenyl)cyclopropane-carbonyl)-5-isobutyl-2-phenyl-imidazolidin-4-one (533)

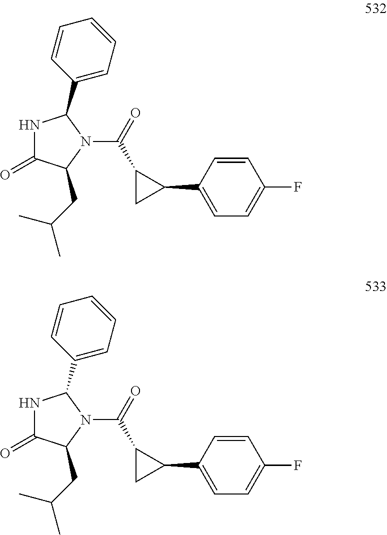

Compound 527 (100 mg, 0.46 mmol) and 60 (100 mg, 0.55 mmol) were coupled according to the procedure described for compound 528/529 to afford 532 (27 mg, 15.5% yield) and 533 (25 mg, 14.5% yield) as an oil.

### Compound 532

¹H NMR (400 MHz, CDCl₃): δ 7.51-7.33 (m, 5H), 7.25-7.14 (d, J=46.4 Hz, 1H), 7.03-6.94 (m, 4H), 6.32 (s, 1H), 4.65-4.31 (m, 1H), 2.43-2.39 (m, 1H), 2.08-1.98 (m, 1H), 1.84-1.65 (m, 3H), 1.52-1.29 (m, 2H), 1.03-0.70 (m, 6H).

MS (ESI): m/z 381.0 [M+H]⁺

### Compound 533

¹H NMR (400 MHz, CDCl₃): δ 7.56-7.20 (m, 6H), 7.05-6.95 (m, 2H), 6.76-6.72 (m, 1H), 6.59-6.56 (m, 1H), 6.39-6.27 (m, 1H), 4.53-4.24 (m, 1H), 2.50-2.40 (m, 1H), 2.12-2.09 (m, 1H), 1.89-1.82 (m, 1H), 1.76-1.10 (m, 2H), 1.05-0.84 (m, 6H).

MS (ESI): m/z 381.0 [M+H]⁺

### Biological Assays

HCV inhibition assay. Clone A or ET-lunet cells were seeded at a density of 1500 or 3000 cells per well in a 96-well plate, respectively. Test compounds serially diluted in culture medium without G418 were added to cells so that the final DMSO concentration was 0.5%. Plates were incubated at 37° C. in a 5% CO₂ atmosphere for 4 days. Inhibition of HCV RNA replication was determined by real time PCR (RT-PCR) or by measuring the levels of luminescence expressed via the luciferase reporter gene encoded within the ET replicon. Briefly, for the RT-PCR assay, total RNA was extracted using the RNeasy-96 kit as recommended by the manufacturer (Qiagen, Valencia, Calif.), reversed transcribed into cDNA, and amplified using primer and probe mix for HCV 5′-NTR RNA and human ribosomal RNA (rRNA) in a one-step RT-PCR reaction as described previously (50). A relative quantification method was used to determine the extent of inhibition. The threshold cycle (Cₜ) of rRNA was subtracted from the Cₜ of HCV RNA (ΔCt). The average ΔCₜ of the DMSO cell controls was then subtracted from the ΔCₜ of the compound treated sample (ΔΔCt). Percent inhibition was determined by using the following equation: %=(1−(2^{-ΔΔC}^{ₜ}))×100. For the luciferase-based replicon assay, luminescence was measured by using a Victor3 plate reader (Perkin-Elmer, Boston, Mass.) following the addition of Bright-Glo reagent as recommended by the manufacturer (Promega, Madison, Wis.). Percent inhibition of HCV replication was determined by comparing the change in luminescence of the drug treated wells versus the DMSO cell controls. EC₅₀ and EC₉₀ values, the concentrations at which 50% and 90% inhibition were achieved, were determined using the GraphPad Prism software (San Diego, Calif.).

Cell cytotoxicity assays. Each compound (serially diluted from 100 μM) was added to Huh7 (2×10³ cells/well), HepG2 (2×10³ cells/well), BxPC3 (2×10³ cells/well), or CEM (5×10³ cells/well) cells and allowed to incubate for 8 days at 37° C. A medium only control was used to determine the minimum absorbance value and an untreated cell. At the end of the growth period, MTS dye from the CellTiter 96 Aqueous One Solution Cell Proliferation Assay kit (Promega) was added to each well and the plate was incubated for an additional 2 hours. The absorbance at 490 nm was read with a Victor3 plate reader (Perkin Elmer) using the medium only control wells as blanks. The 90% inhibition value (CC₉₀) was determined by comparing the absorbance in wells containing cells and test compound to untreated cell control wells.

TABLE 1
HCV Replicon 1b Screening Data Table

| | | Replicon 1b | |
|---|---|---|---|
| Compound | | potency | Cytotoxicity |
| Number | Structure | EC₉₀(μM) | CC₉₀(μM) |
| 70 | 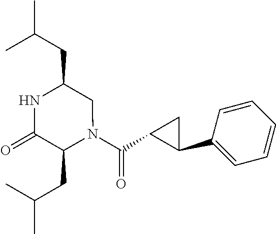 | 0.80 | >10 |
| 71 | 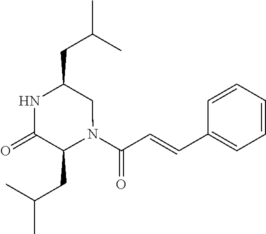 | 3.17 | >10 |
| 72 | 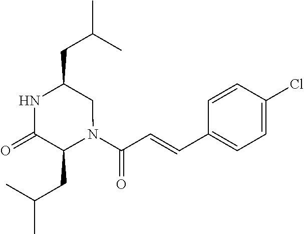 | 2.50 | >10 |
| 73 | 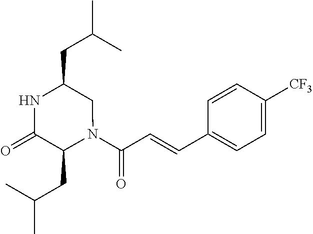 | 3.34 | >10 |
| 74 | 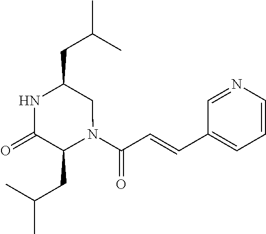 | >10 | >10 |
| 75 | 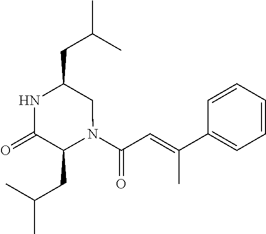 | >10 | >10 |
| 76 | 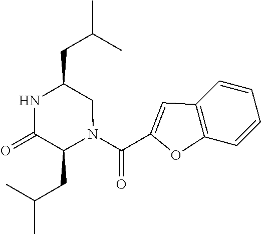 | 9.18 | >10 |
| 77 | 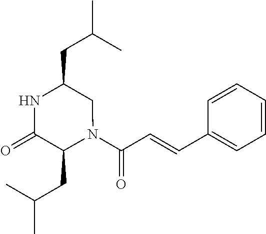 | 3.24 | >10 |
| 78 | 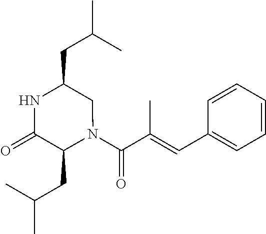 | 9.93 | >10 |
| 79 | 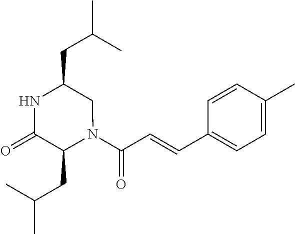 | 5.29 | >10 |
| 80 | 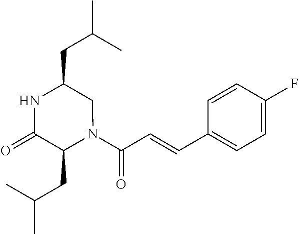 | 2.49 | >10 |
| 81 | 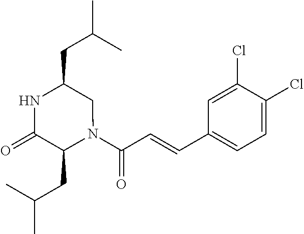 | 7.31 | >10 |
| 82 | 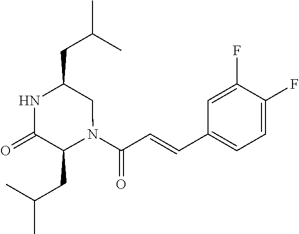 | 1.22 | >10 |
| 83 | 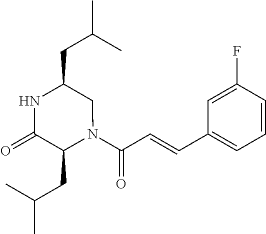 | 2.91 | >10 |
| 84 | 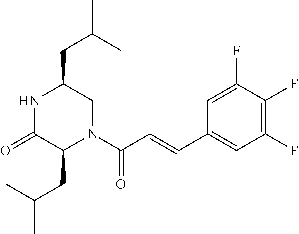 | 2.64 | >10 |
| 85 | 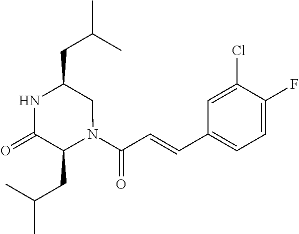 | 8.65 | >10 |
| 86 | 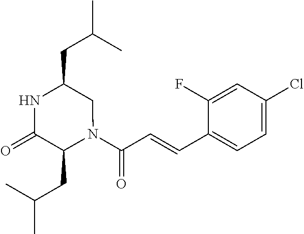 | 0.59 | >10 |
| 87 | 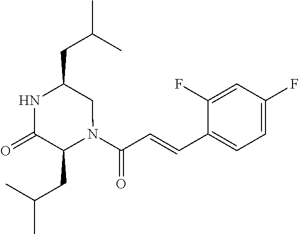 | 0.57 | >10 |
| 88 | 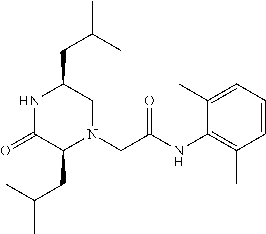 | >10 | >10 |
| 89 | 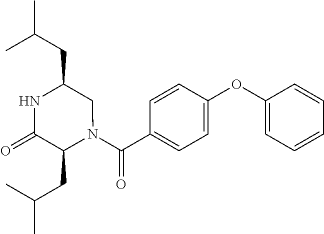 | 6.88 | >10 |
| 90 | 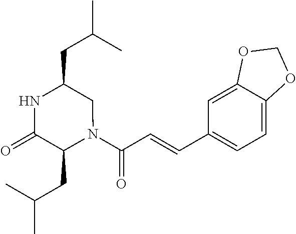 | 7.72 | >10 |
| 91 | 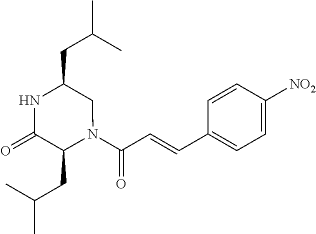 | >10 | >10 |
| 92 | 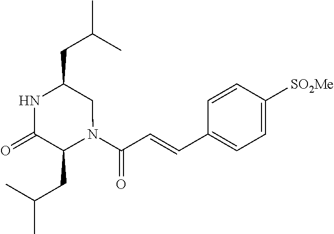 | >10 | >10 |
| 93 | 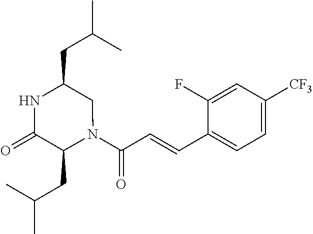 | 9.62 | >10 |
| 94 | 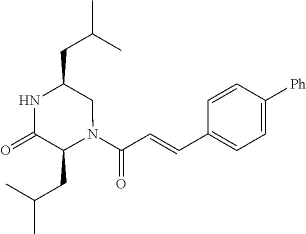 | 8.37 | >10 |
| 95 | 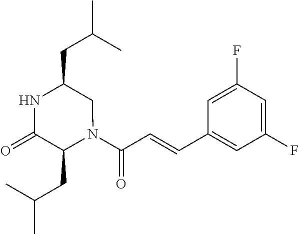 | 9.31 | >10 |
| 96 | 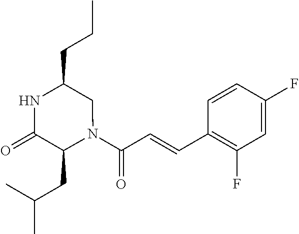 | 1.11 | >10 |
| 97 | 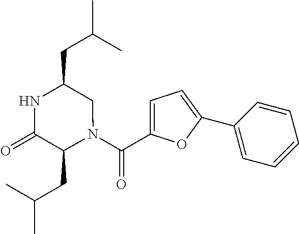 | 7.28 | >10 |
| 98 | 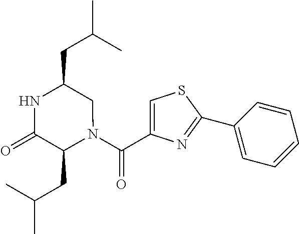 | 7.92 | >10 |
| 99 | 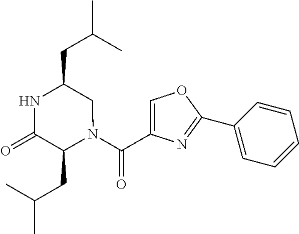 | >10 | >10 |
| 100 | 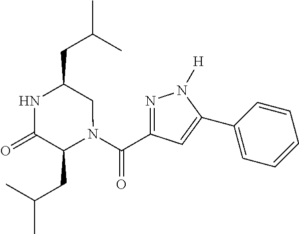 | >10 | >10 |
| 101 | 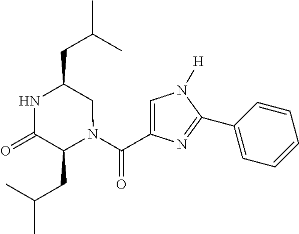 | >10 | >10 |
| 102 | 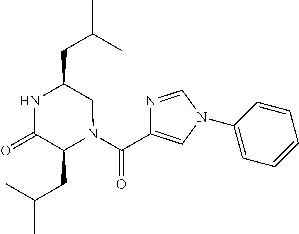 | >10 | >10 |
| 103 | 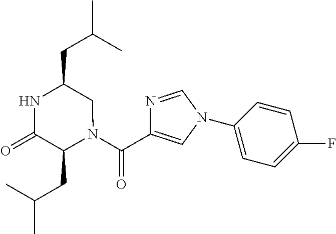 | 8.64 | >10 |
| 104 | 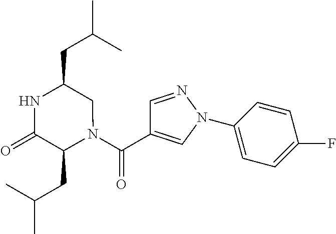 | 4.74 | >10 |
| 105 | 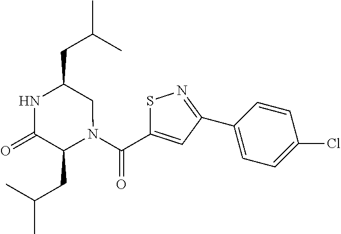 | 1.49 | >10 |
| 106 | 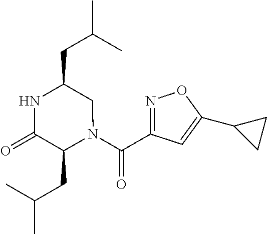 | 8.77 | >10 |
| 107 | 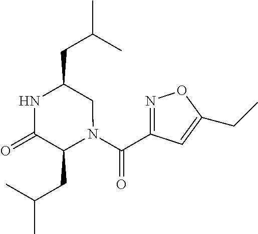 | 8.41 | >10 |
| 108 | 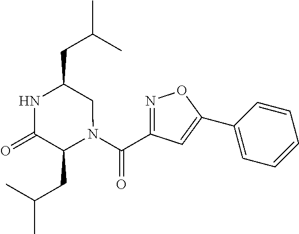 | 0.52 | >10 |
| 109 | 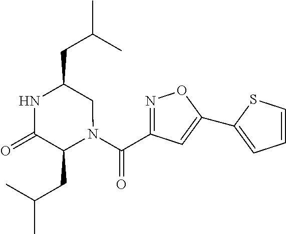 | 0.54 | >10 |
| 110 | 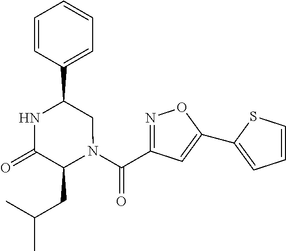 | 0.59 | >10 |
| 111 | 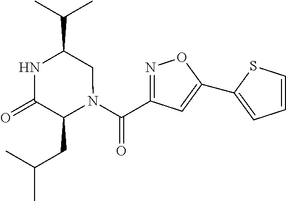 | 0.80 | >10 |
| 112 | 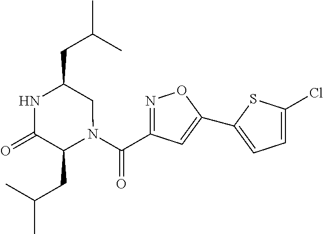 | 0.95 | >10 |
| 113 | 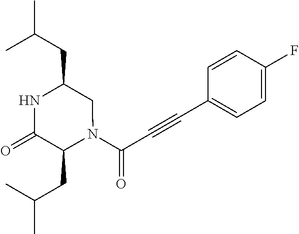 | 3.40 | >10 |
| 114 | 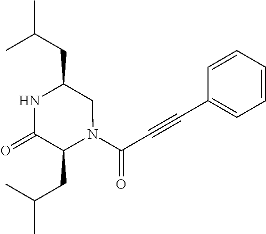 | 4.08 | >10 |
| 115 | 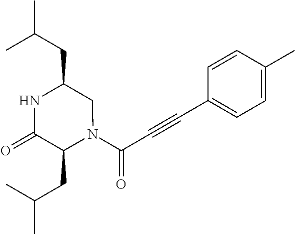 | 5.01 | >10 |
| 116 | 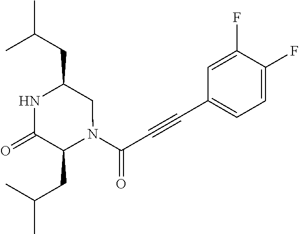 | 9.65 | >10 |
| 117 | 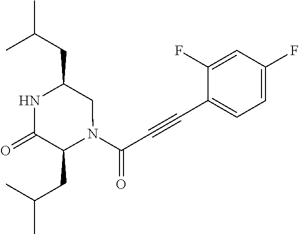 | 2.66 | >10 |
| 118 | 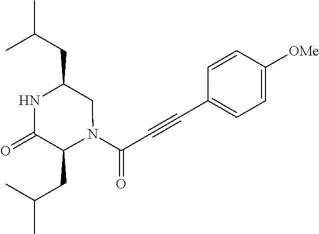 | 2.96 | >10 |
| 119 | 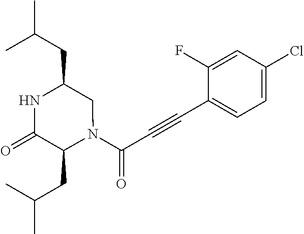 | 6.20 | >10 |
| 120 | 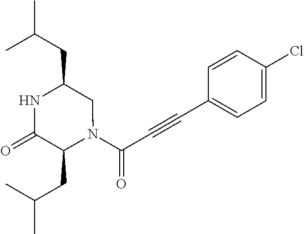 | 9.16 | >10 |
| 121 | 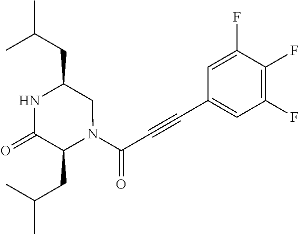 | 3.23 | >10 |
| 122 | 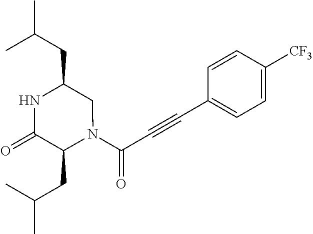 | 7.54 | >10 |
| 123 | 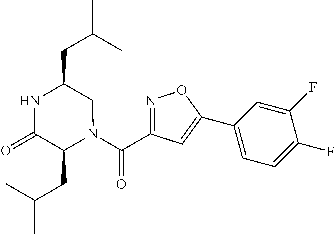 | 0.97 | >10 |
| 124 | 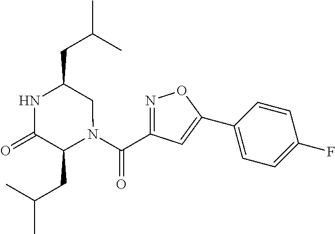 | 0.11 | >10 |
| 125 | 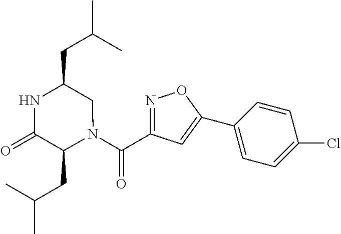 | 0.26 | >10 |
| 126 | 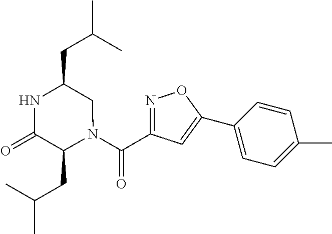 | 1.66 | >10 |
| 127 | 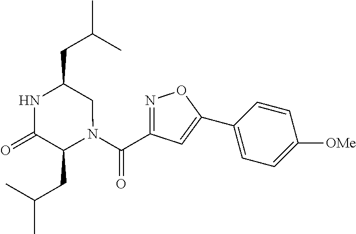 | 1.14 | >10 |
| 128 | 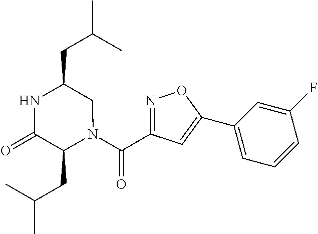 | 0.84 | >10 |
| 129 | 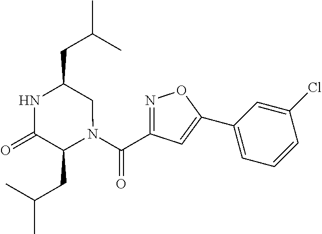 | 2.93 | >10 |
| 130 | 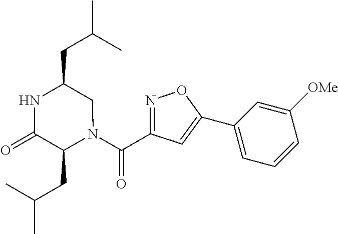 | 5.75 | >10 |
| 131 | 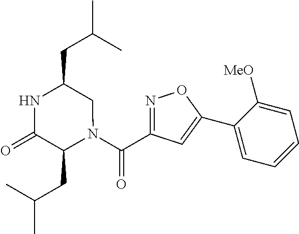 | 3.49 | >10 |
| 132 | 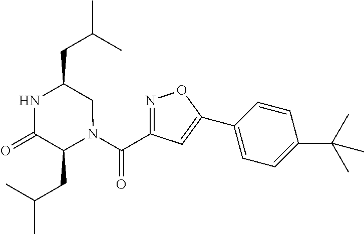 | 4.33 | >10 |
| 133 | 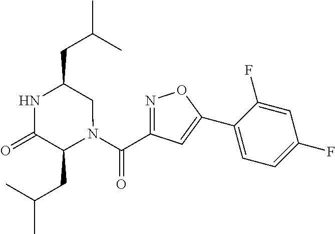 | 0.60 | >10 |
| 134 | 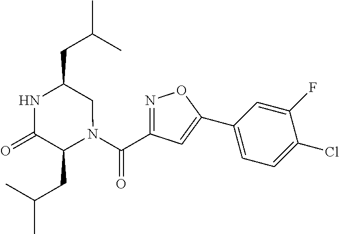 | 0.89 | >10 |
| 135 | 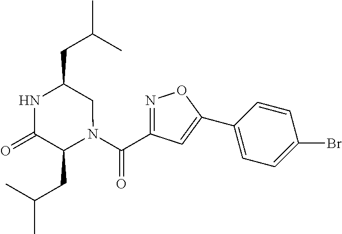 | 0.47 | >10 |
| 136 | 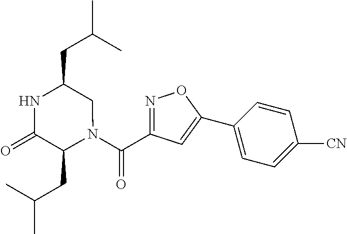 | 1.53 | >10 |
| 137 | 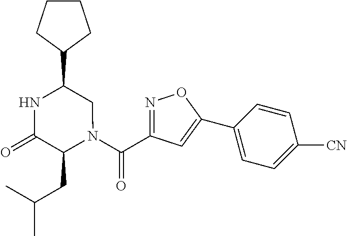 | 0.80 | >10 |
| 138 | 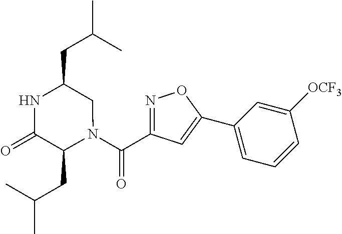 | 7.04 | >10 |
| 139 | 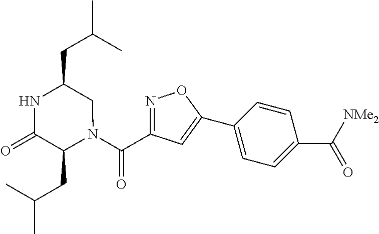 | >10 | >10 |
| 140 | 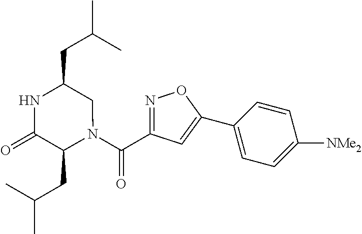 | >10 | >10 |
| 141 | 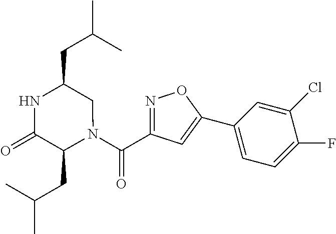 | 1.78 | >10 |
| 142 | 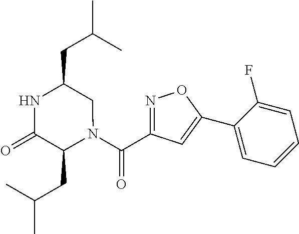 | 2.01 | >10 |
| 143 | 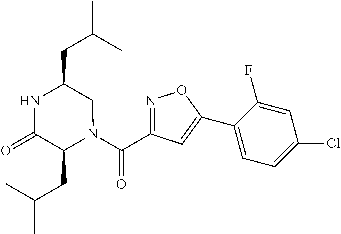 | 1.21 | >10 |
| 144 | 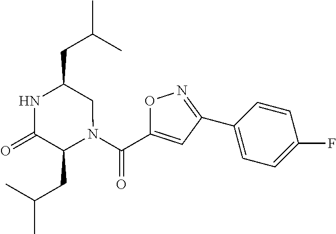 | 0.33 | >10 |
| 145 | 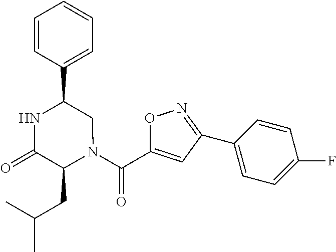 | 0.23 | >10 |
| 146 | 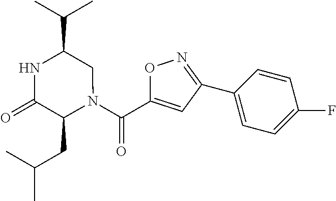 | 0.73 | >10 |
| 147 | 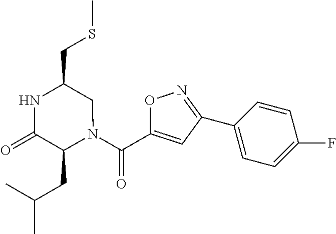 | 1.27 | >10 |
| 148 | 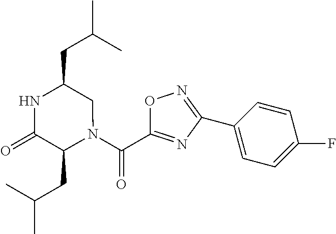 | 0.37 | >10 |
| 149 | 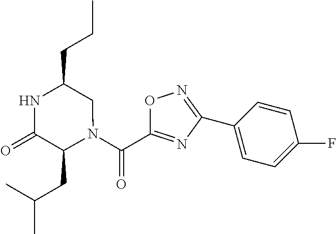 | 0.05 | >10 |
| 150 | 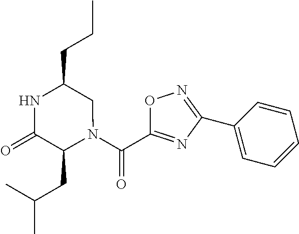 | 0.59 | >10 |
| 151 | 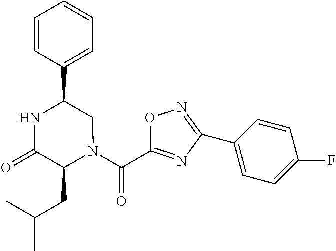 | 0.04 | >10 |
| 152 | 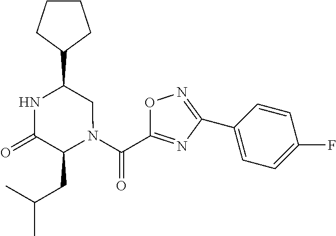 | 0.11 | >10 |
| 153 | 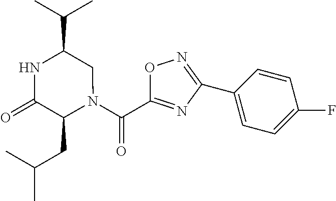 | 0.22 | >10 |
| 154 | 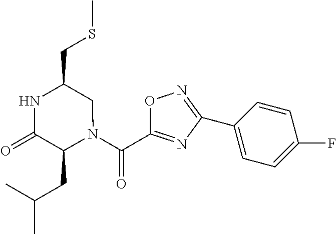 | 0.78 | >10 |
| 155 | 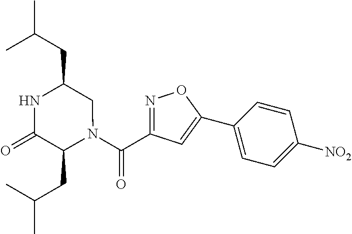 | 2.60 | >10 |
| 156 | 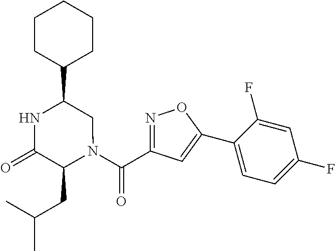 | 1.15 | >10 |
| 157 | 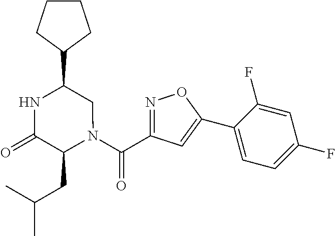 | 0.49 | >10 |
| 158 | 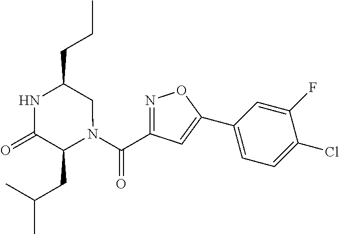 | 0.44 | >10 |
| 159 | 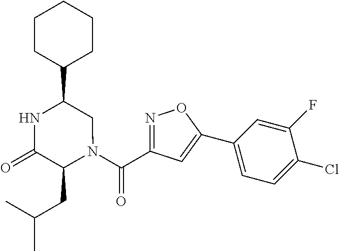 | 0.71 | >10 |
| 160 | 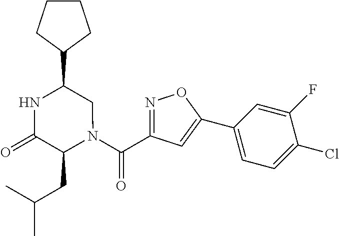 | 0.41 | >10 |
| 161 | 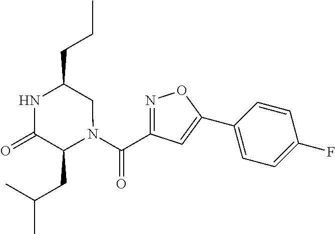 | 0.07 | >10 |
| 162 | 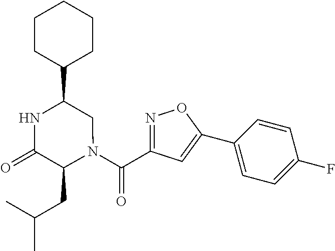 | 1.8 | >10 |
| 163 | 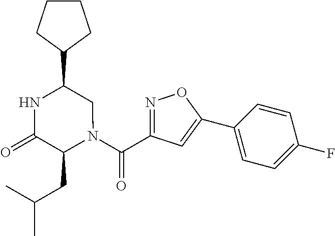 | 0.09 | >10 |
| 164 | 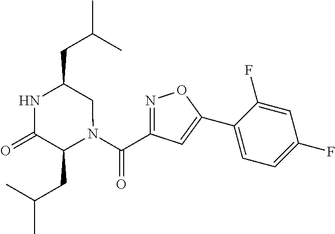 | 0.68 | >10 |
| 165 | 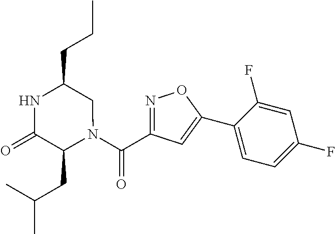 | 0.34 | >10 |
| 166 | 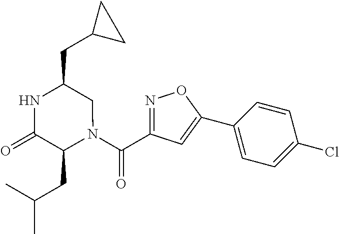 | 0.36 | >10 |
| 167 | 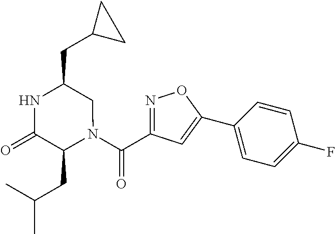 | 0.45 | >10 |
| 168 | 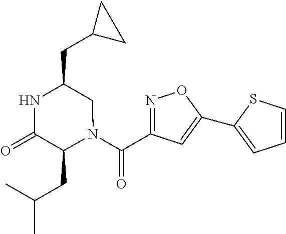 | 0.90 | >10 |
| 169 | 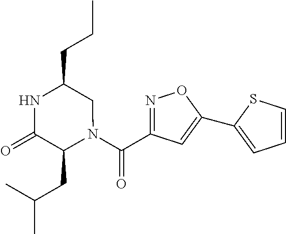 | 0.15 | >10 |
| 170 | 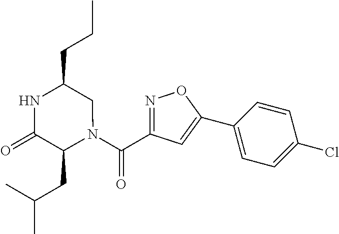 | 0.05 | >10 |
| 171 | 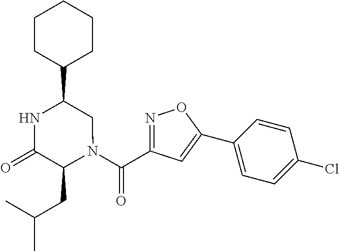 | 0.15 | >10 |
| 172 | 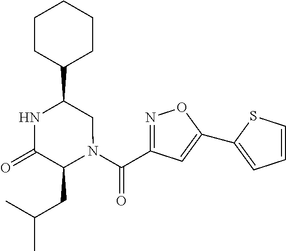 | 0.41 | >10 |
| 173 | 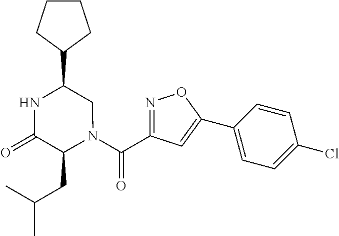 | 0.12 | >10 |
| 174 | 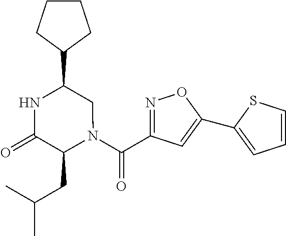 | 0.25 | >10 |
| 175 | 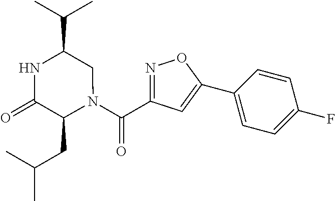 | 0.17 | >10 |
| 176 | 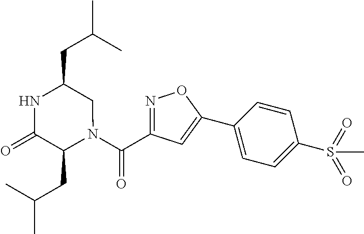 | >20 | >10 |
| 177 | 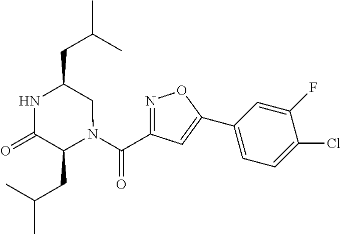 | 0.89 | >10 |
| 178 | 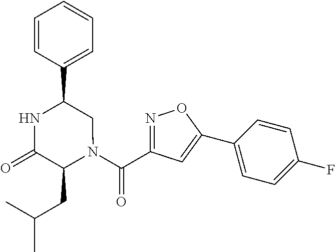 | 0.05 | >10 |
| 179 | 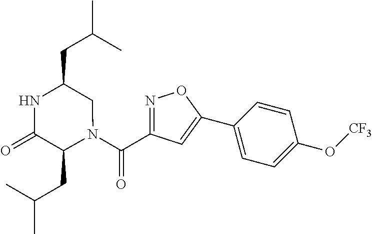 | 14.9 | >10 |
| 180 | 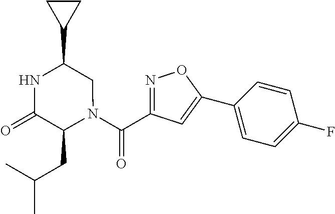 | 0.60 | >10 |
| 181 | 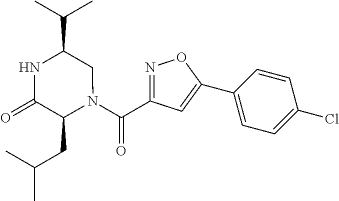 | 0.23 | >10 |
| 182 | 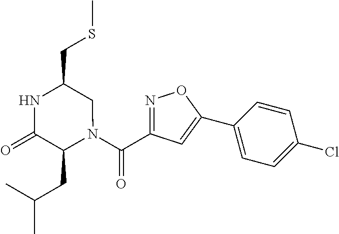 | 0.55 | >10 |
| 183 | 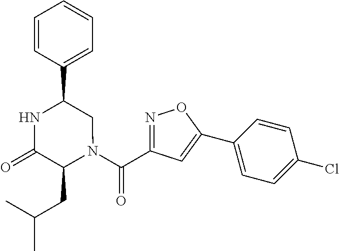 | 0.06 | >10 |
| 184 | 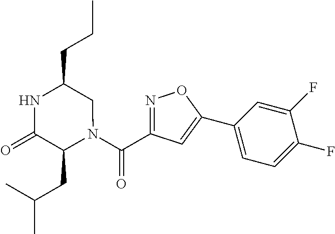 | 0.14 | >10 |
| 185 | 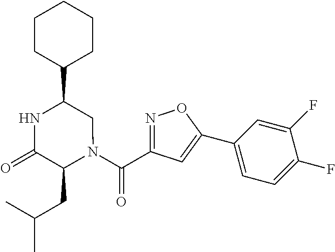 | 0.61 | >10 |
| 186 | 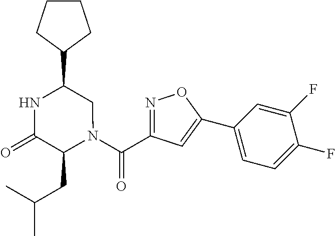 | 0.26 | >10 |
| 187 | 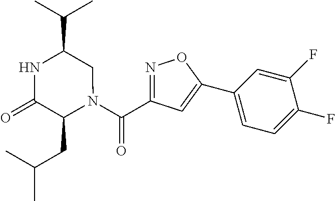 | 0.62 | >10 |
| 188 | 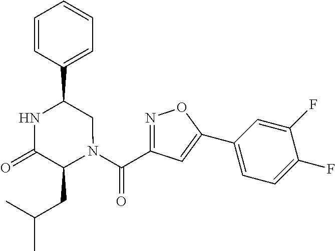 | 0.18 | >10 |
| 189 | 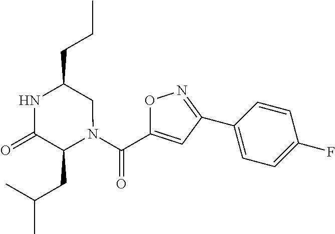 | 0.11 | >10 |
| 190 | 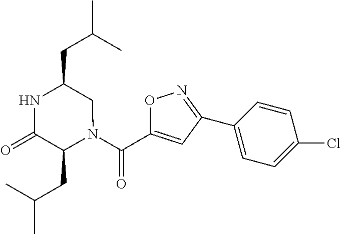 | 0.32 | >10 |
| 191 | 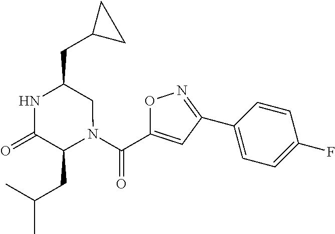 | 0.12 | >10 |
| 192 | 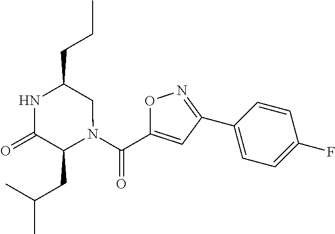 | 0.11 | >10 |
| 193 | 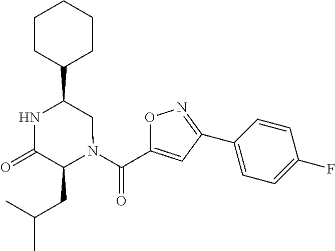 | 0.05 | >10 |
| 194 | 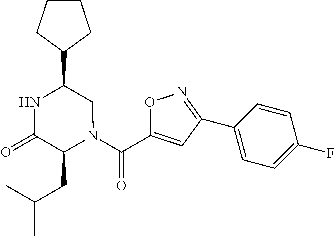 | 0.04 | >10 |
| 195 | 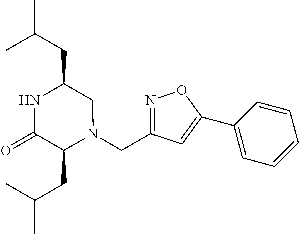 | 0.75 | >10 |
| 196 | 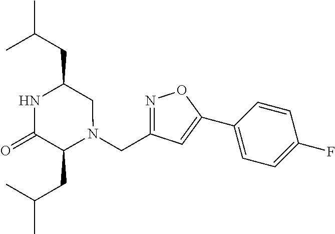 | 0.08 | >10 |
| 197 | 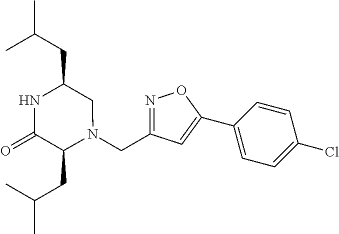 | 0.24 | >10 |
| 198 | 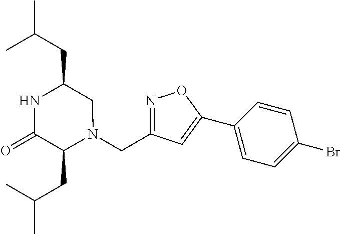 | 0.55 | >10 |
| 199 | 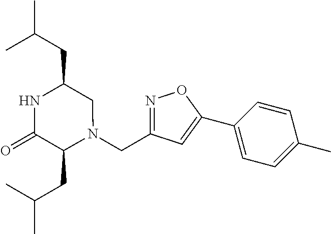 | 2.15 | >10 |
| 200 | 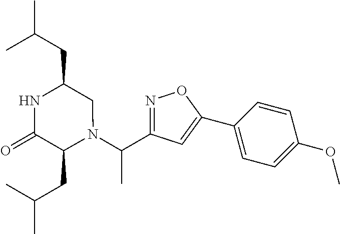 | 5.79 | >10 |
| 201 | 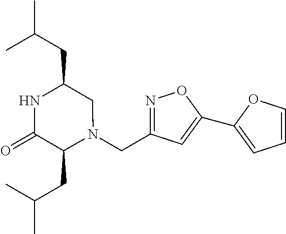 | 1.82 | >10 |
| 202 | 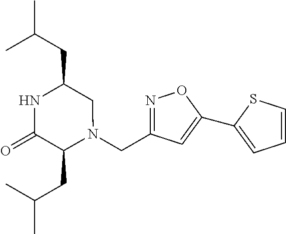 | 0.88 | >10 |
| 203 | 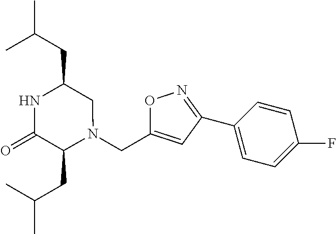 | 0.69 | >10 |
| 204 | 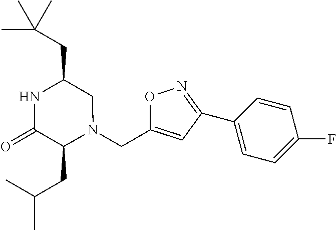 | 1.33 | >10 |
| 205 | 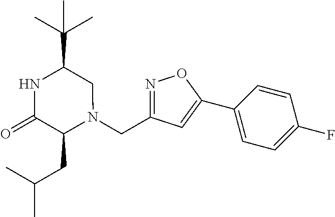 | 8.9 | >10 |
| 206 | 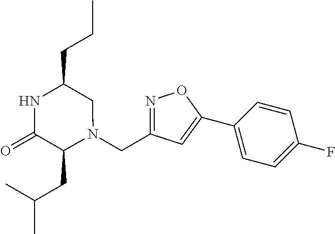 | 0.21 | >10 |
| 207 | 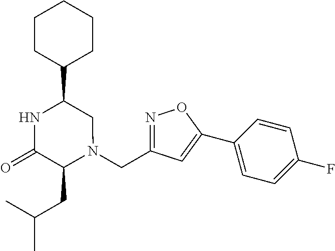 | 0.86 | >10 |
| 208 | 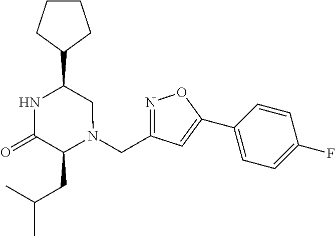 | 0.21 | >10 |
| 209 | 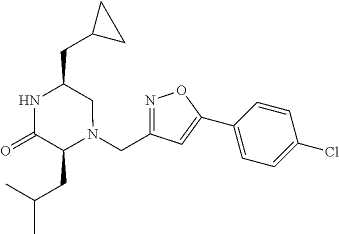 | 0.67 | >10 |
| 210 | 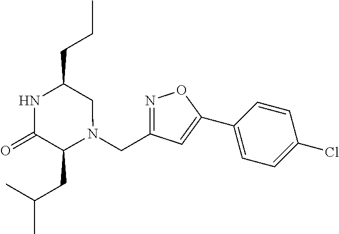 | 0.32 | >10 |
| 211 | 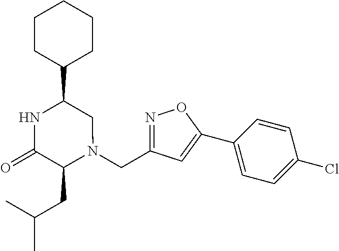 | 0.41 | >10 |
| 212 | 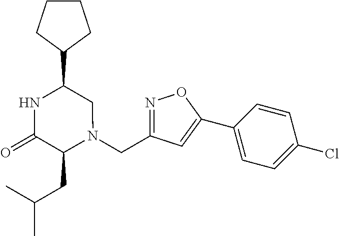 | 0.26 | >10 |
| 213 | 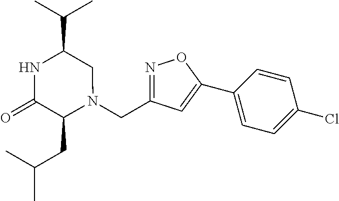 | 0.74 | >10 |
| 214 | 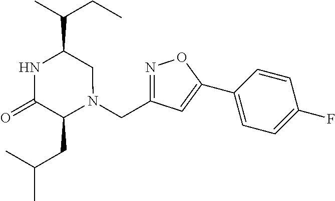 | 0.72 | >10 |
| 215 | 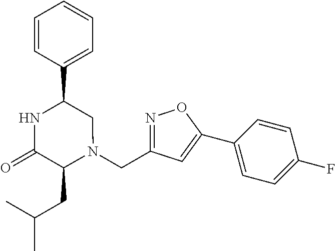 | 0.19 | >10 |
| 216 | 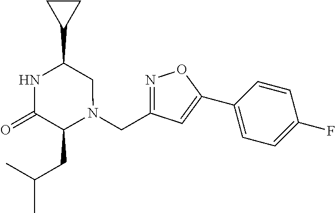 | 1.60 | >10 |
| 217 | 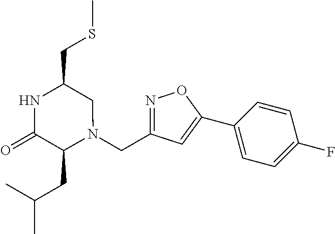 | 0.82 | >10 |
| 218 | 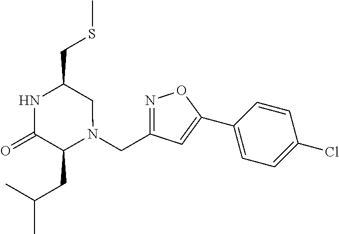 | 0.75 | >10 |
| 219 | 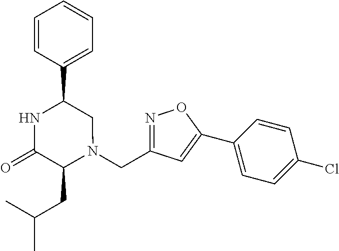 | 4.5 | >10 |
| 220 | 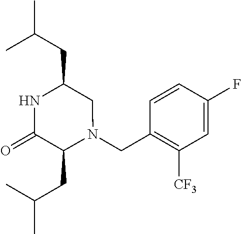 | 8.35 | >10 |
| 221 | 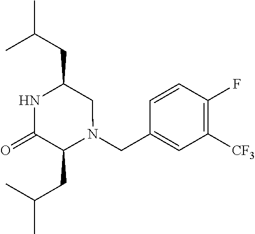 | >10 | >10 |
| 222 | 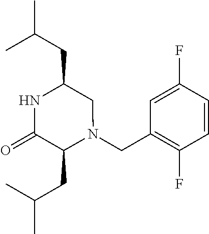 | 9.70 | >10 |
| 223 | 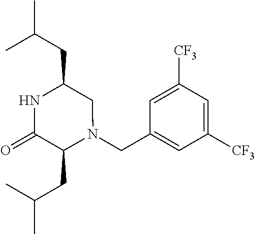 | 4.49 | >10 |
| 224 | 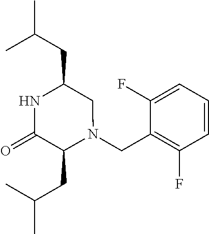 | 8.36 | >10 |
| 225 | 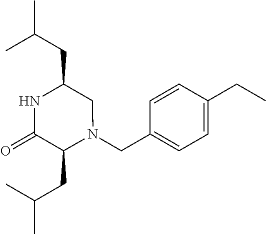 | 3.51 | >10 |
| 226 | 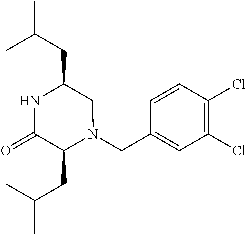 | 8.87 | >10 |
| 227 | 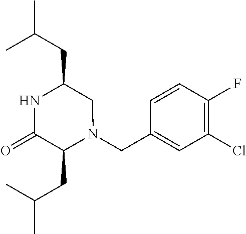 | >10 | >10 |
| 228 | 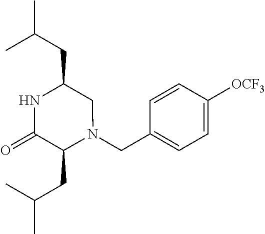 | 3.26 | >10 |
| 229 | 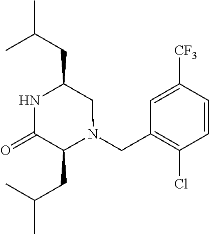 | 5.12 | >10 |
| 230 | 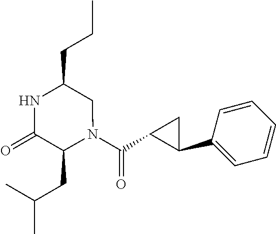 | 0.57 | >10 |
| 231 | 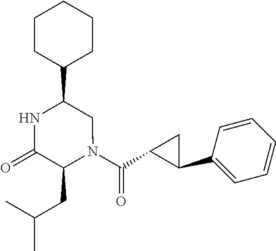 | 0.93 | >10 |
| 232 | 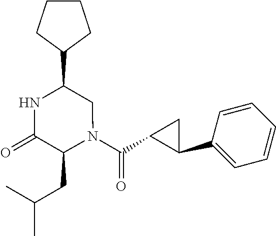 | 0.69 | >10 |
| 233 | 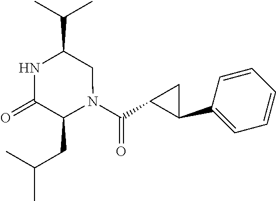 | 0.93 | >10 |
| 234 | 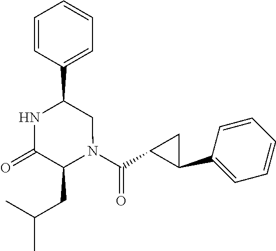 | 0.30 | >10 |
| 235 | 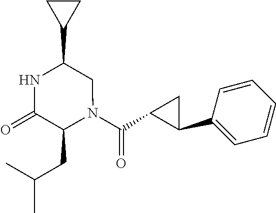 | 4.2 | >10 |
| 236 | 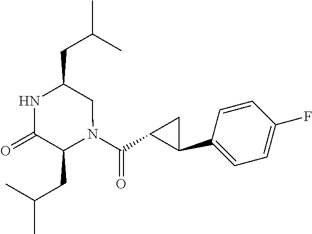 | 0.08 | >10 |
| 237 | 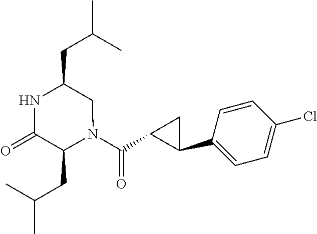 | 0.27 | >10 |
| 238 | 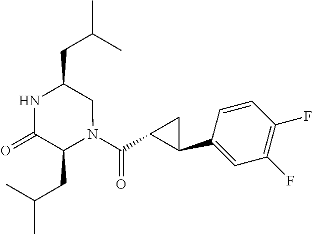 | 0.59 | >10 |
| 239 | 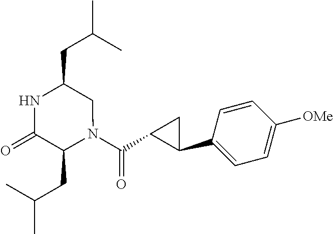 | 0.96 | >10 |
| 240 | 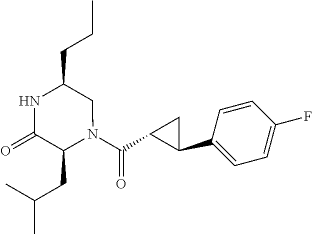 | 0.10 | >10 |
| 241 | 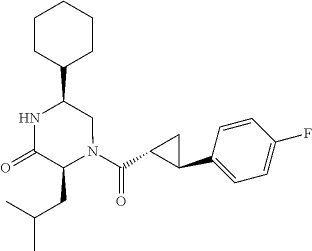 | 0.15 | >10 |
| 242 | 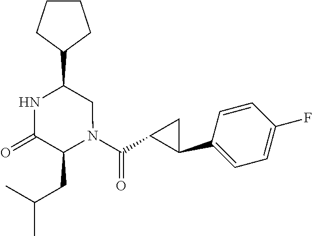 | 0.06 | >10 |
| 243 | 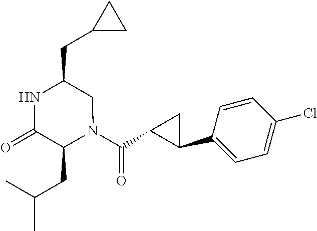 | 0.43 | >10 |
| 244 | 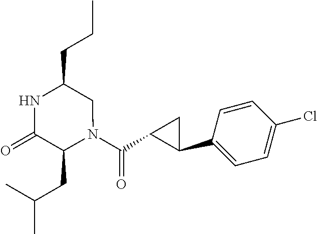 | 0.07 | >10 |
| 245 | 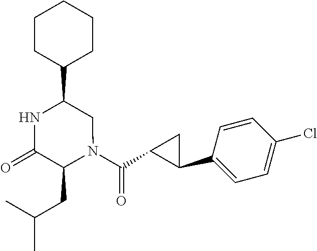 | 0.62 | >10 |
| 246 | 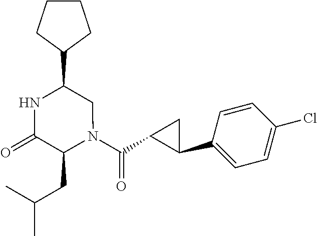 | 0.20 | >10 |
| 247 | 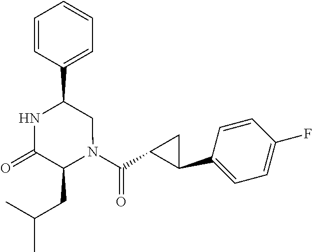 | 0.05 | >10 |
| 248 | 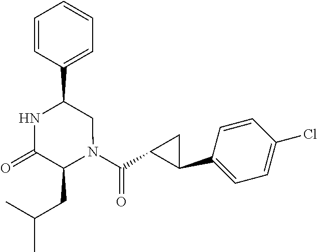 | 0.16 | >10 |
| 249 | 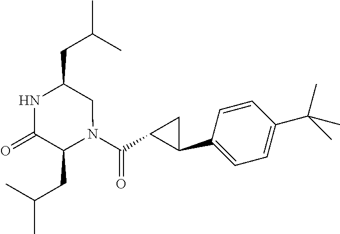 | 0.75 | >10 |
| 250 | 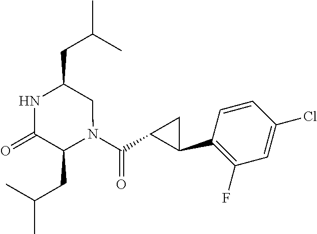 | 0.36 | >10 |
| 251 | 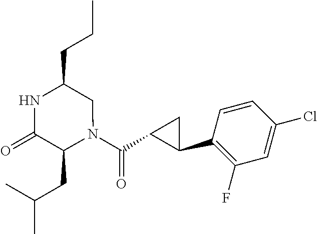 | 0.20 | >10 |
| 252 | 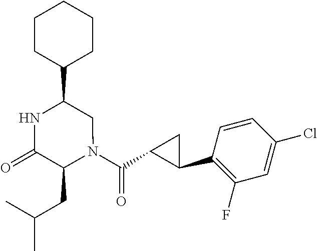 | 0.67 | >10 |
| 253 | 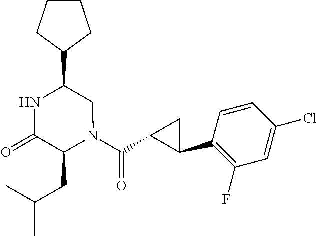 | 0.50 | >10 |
| 254 | 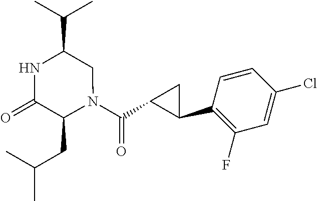 | 0.66 | >10 |
| 255 | 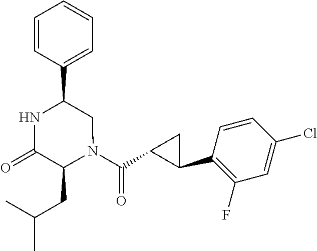 | 0.36 | >10 |
| 256 | 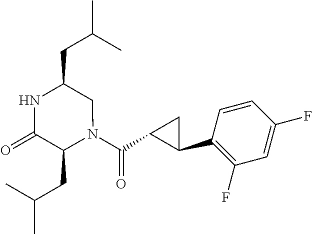 | 0.22 | >10 |
| 257 | 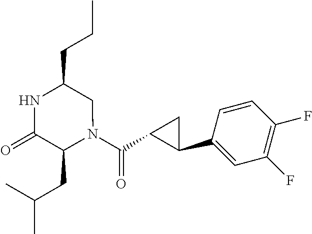 | 0.59 | >10 |
| 258 | 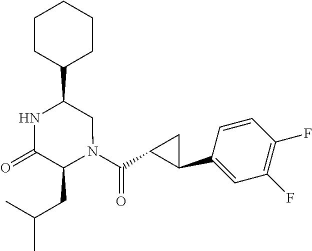 | 0.56 | >10 |
| 259 | 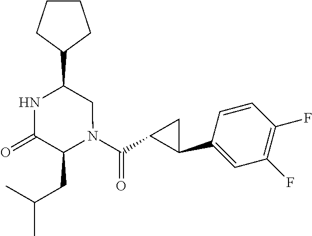 | 0.59 | >10 |
| 260 | 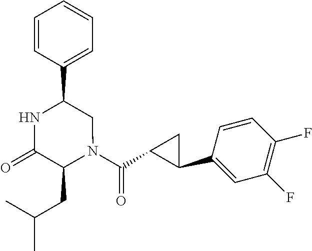 | 0.23 | >10 |

Cell Line and Maintenance: The genotype (GT) 1a H77 replicon (Apath LLC Brooklyn, N.Y.) was stably expressed in Huh7-Lunet cells (kindly provided by R. Bartenschlager University of Heidelberg, Heidelberg, Germany) and maintained in Dulbecco's modified Eagle's medium supplemented with 10% fetal bovine serum, 0.1 mM non essential amino acids (NEAA), 4 mM L-glutamine, 1 mM sodium pyruvate and 0.750 mg/mL G418 (Geneticin) (Invitrogen, Carlsbad, Calif.).

Replicon Assay: Inhibition of HCV in the H77 GT 1a replicon (Apath LLC, Brooklyn, N.Y.) was measured by Real-Time PCR (RT-PCR) Replicon cells were added to the plate at 1500 cells/well in 50 μL of medium and 50 μL of 2× drug dilutions were added (in duplicate) per well in a 96 well plate to total 100 μL per well. The plate was incubated at 37° C. in a humidified 8% CO₂ atmosphere for 4 days. After incubation, the supernatant was discarded and total RNA was extracted with RNeasy 96 (Qiagen, Valencia Calif.) per manufacturer's instructions. The extracted RNA was amplified as described by Stuyver et al., J. Virol. (2003) 77: 10689-10694, and the ΔCₜ values for HCV were determined and the EC₉₀ was calculated using GraphPad Prism software (San Diego, Calif.). A “no drug” (medium only) control was used to determine maximum amount of HCV RNA.

TABLE 2
Screening data for selected compounds

| | | | RT-PCR | |
|---|---|---|---|---|
| | | Replicon 1b | H77 1a | |
| Compound | | potency | Potency | Cytotoxicity |
| Number | Structure | EC₉₀(μM) | EC₉₀(μM) | CC₉₀(μM) |
| 261 | 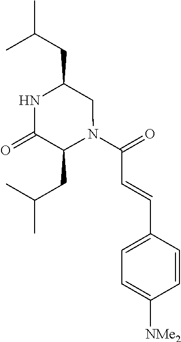 | >10 | | >10 |
| 262 | 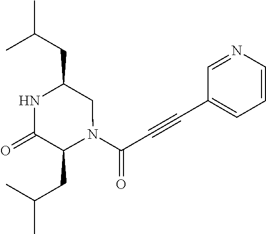 | >10 | | >10 |
| 263 | 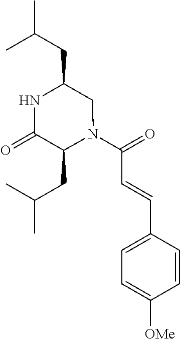 | 8.70 | | >10 |
| 264 | 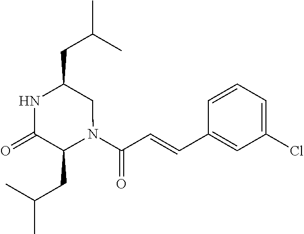 | 8.10 | | >10 |
| 265 | 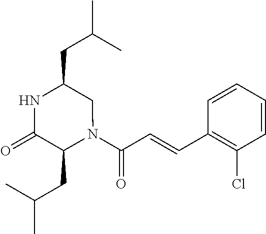 | 9.75 | | >10 |
| 266 | 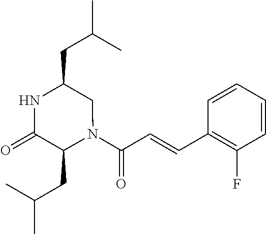 | 7.93 | | >10 |
| 267 | 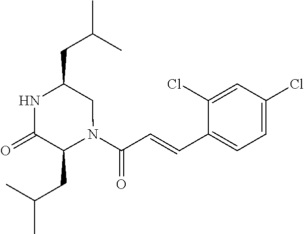 | 6.89 | | >10 |
| 268 | 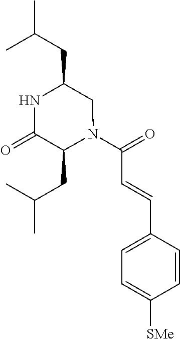 | 9.80 | | >10 |
| 269 | 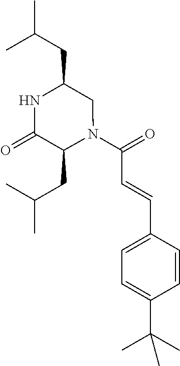 | 8.90 | | >10 |
| 270 | 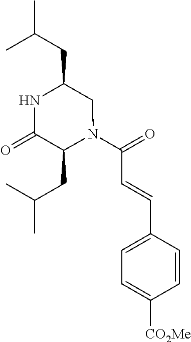 | >10 | | >10 |
| 271 | 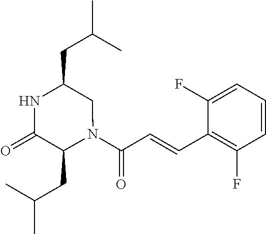 | >10 | | >10 |
| 272 | 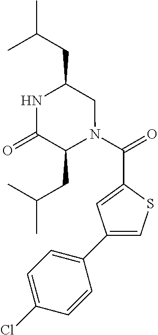 | 3.56 | | >10 |
| 273 | 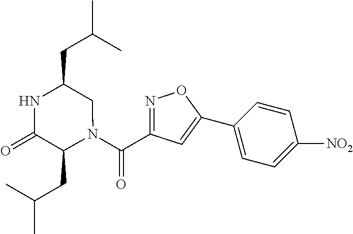 | 3.76 | | >10 |
| 274 | 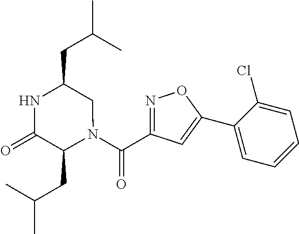 | 5.95 | | >10 |
| 275 | 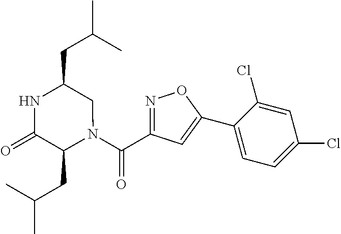 | 6.31 | | >10 |
| 276 | 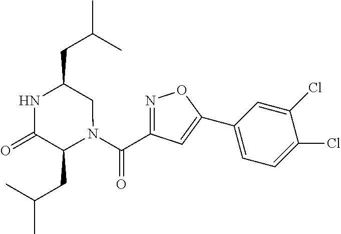 | 2.98 | | >10 |
| 277 | 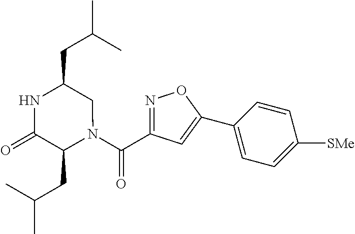 | 5.84 | | >10 |
| 278 | 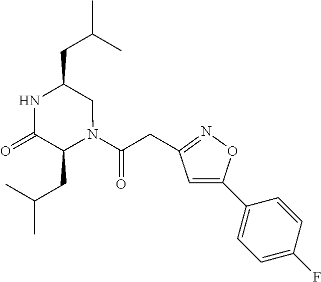 | 7.89 | | >10 |
| 279 | 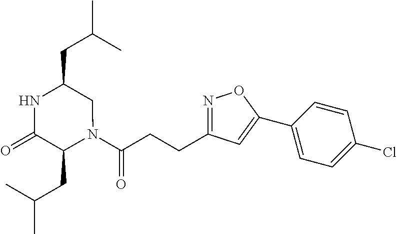 | 7.13 | | >10 |
| 280 | 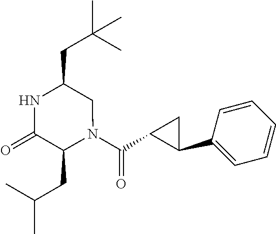 | 8.65 | | >10 |
| 281 | 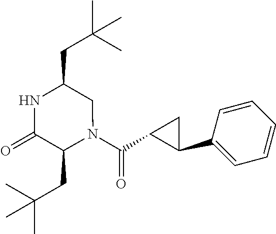 | >10 | | >10 |
| 283 | 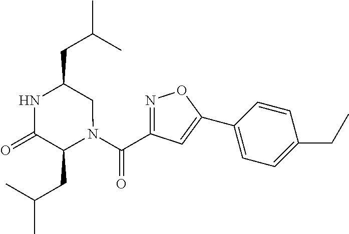 | 8.75 | | >10 |
| 284 | 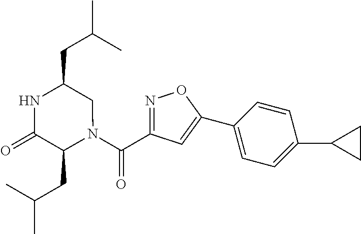 | >10 | | >10 |
| 285 | 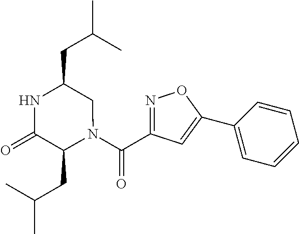 | >10 | | >10 |
| 286 | 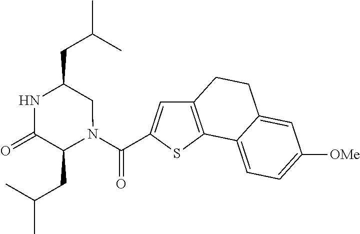 | 3.91 | | >10 |
| 287 | 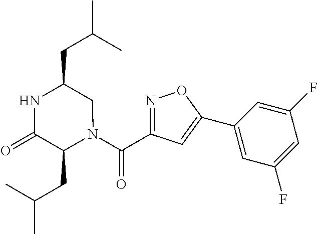 | 3.57 | | >10 |
| 288 | 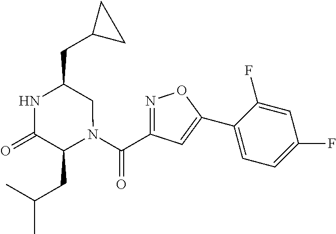 | 1.60 | | >10 |
| 289 | 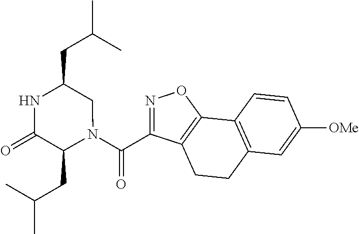 | 5.01 | | >10 |
| 290 | 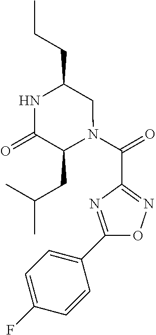 | 0.14 | | >10 |
| 291 | 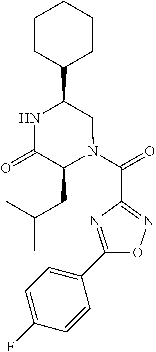 | 0.33 | | >10 |
| 292 | 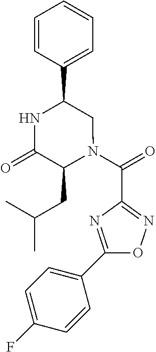 | 0.078 | 0.079 | >10 |
| 293 | 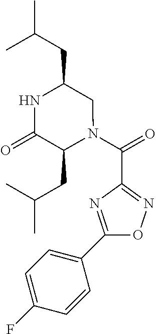 | 0.27 | | >10 |
| 294 | 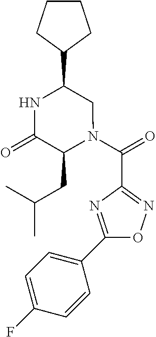 | 0.35 | | >10 |
| 295 | 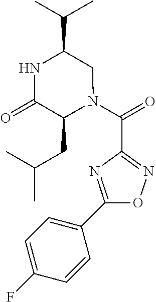 | 0.32 | | >10 |
| 296 | 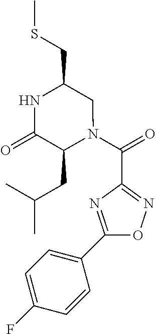 | 1.54 | | >10 |
| 297 | 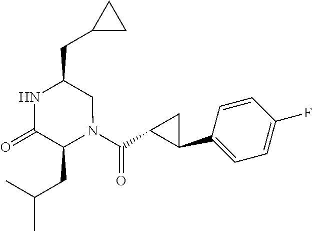 | 0.41 | | >10 |
| 298 | 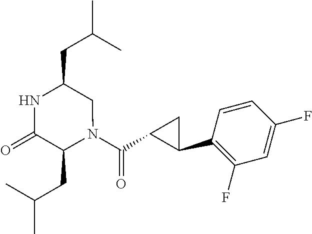 | 0.23 | | >10 |
| 299 | 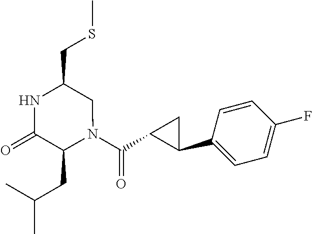 | 0.63 | | >10 |
| 300 | 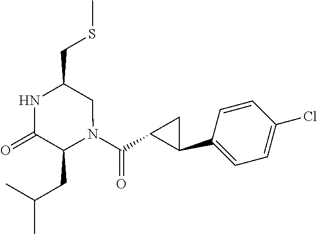 | 0.78 | | >10 |
| 301 | 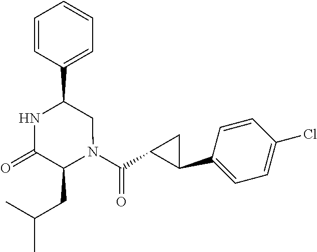 | 0.17 | | >10 |
| 302 | 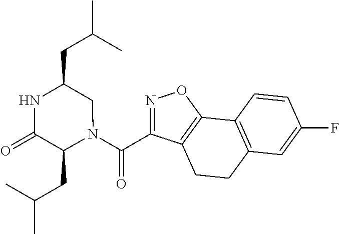 | 1.41 | | >10 |
| 304 | 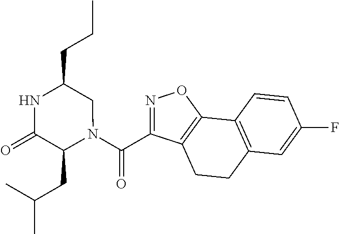 | 0.64 | | >10 |
| 305 | 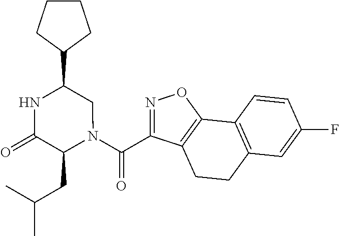 | 1.61 | | >10 |
| 306 | 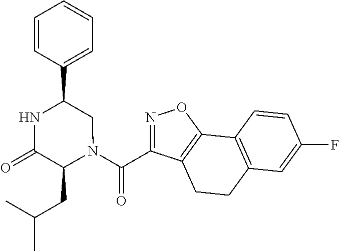 | 0.89 | | >10 |
| 307 | 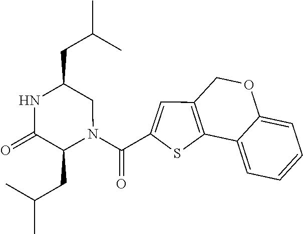 | 4.71 | | >10 |
| 308 | 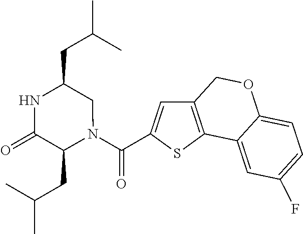 | 6.48 | | >10 |
| 309 | 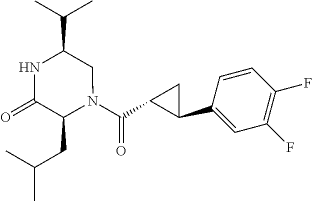 | 0.78 | | >10 |
| 310 | 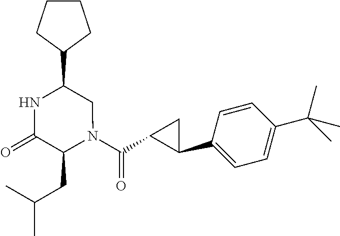 | 0.95 | | >10 |
| 311 | 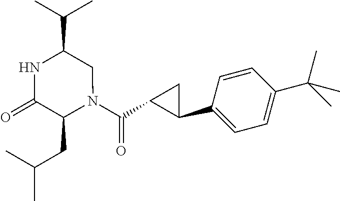 | 1.86 | | >10 |
| 312 | 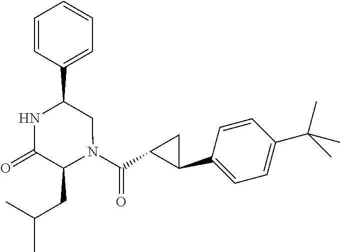 | 1.13 | | >10 |
| 313 | 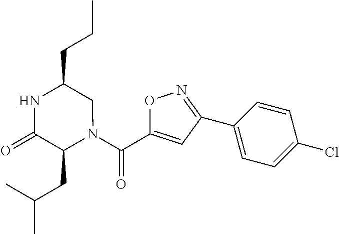 | 0.39 | | >10 |
| 314 | 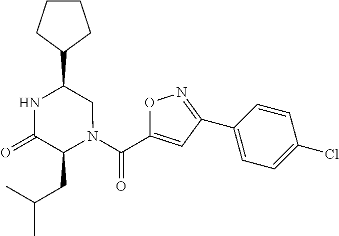 | 0.51 | | >10 |
| 315 | 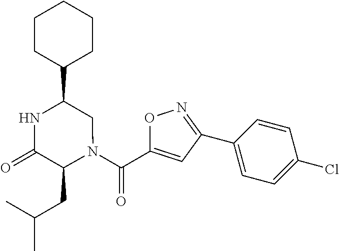 | 0.70 | | >10 |
| 316 | 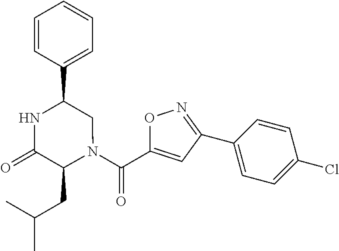 | 0.28 | | >10 |
| 317 | 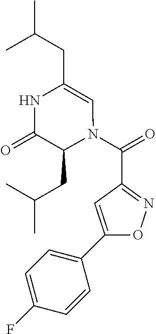 | 0.28 | | >10 |
| 321 | 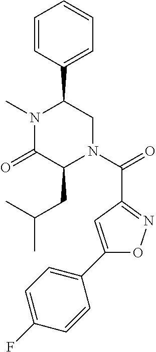 | 6.45 | | >10 |
| 322 | 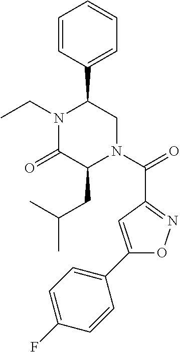 | >10 | | >10 |
| 323 | 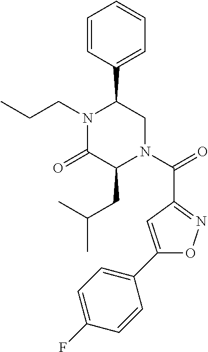 | >10 | | >10 |
| 324 | 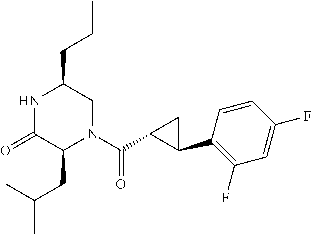 | >10 | | >10 |
| 325 | 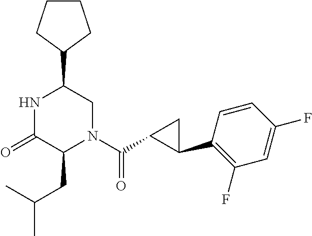 | >10 | | >10 |
| 326 | 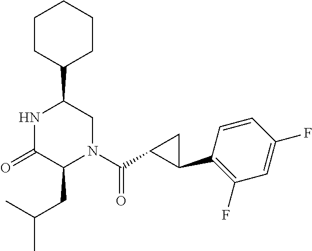 | >10 | | >10 |
| 327 | 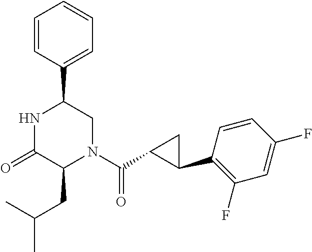 | 7.21 | | >10 |
| 328 | 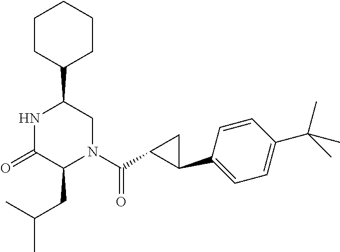 | 2.43 | | >10 |
| 329 | 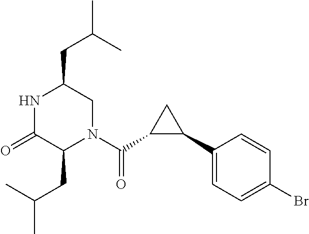 | 0.52 | | >10 |
| 331 | 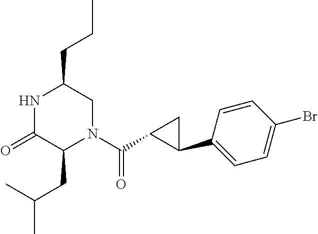 | 0.56 | | >10 |
| 332 | 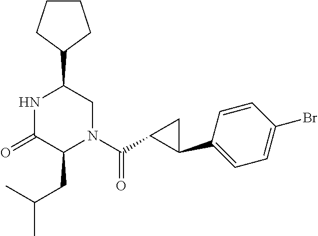 | 1.31 | | |
| 333 | 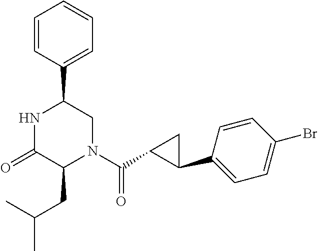 | 0.646 | | >10 |
| 334 | 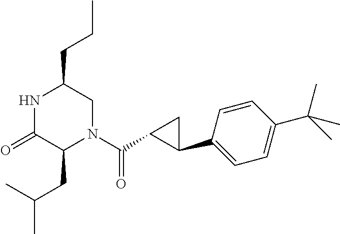 | 0.90 | | >10 |
| 335 | 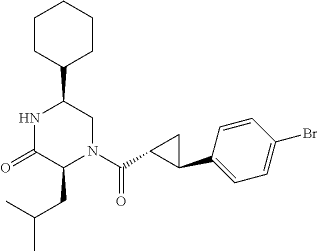 | 4.38 | | >10 |
| 336 | 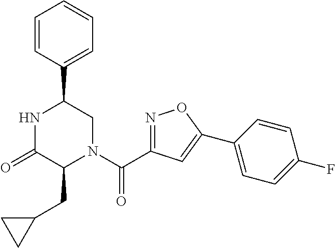 | 0.36 | | >10 |
| 338 | 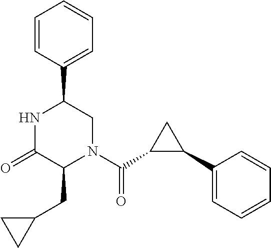 | 5.07 | | >10 |
| 339 | 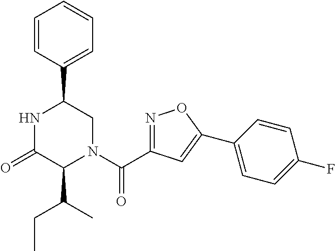 | 0.80 | | >10 |
| 341 | 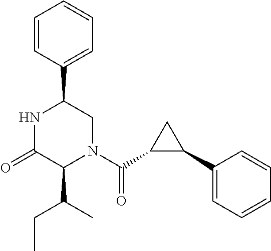 | 2.54 | | >10 |
| 342 | 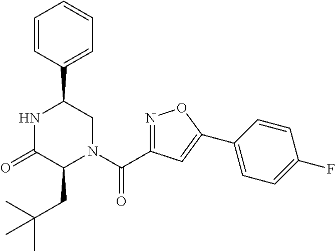 | 0.20 | | >10 |
| 344 | 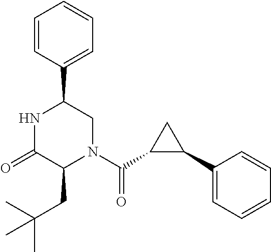 | 1.00 | | >10 |
| 345 | 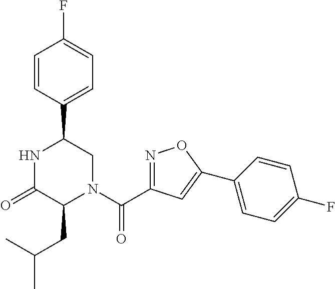 | 0.65 | | >10 |
| 347 | 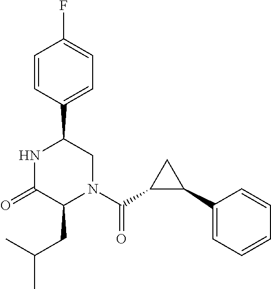 | 2.05 | | >10 |
| 348 | 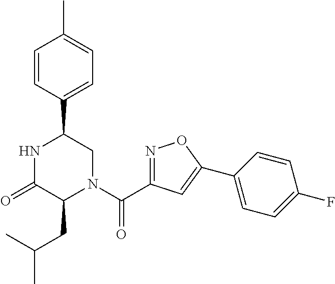 | 1.80 | | >10 |
| 350 | 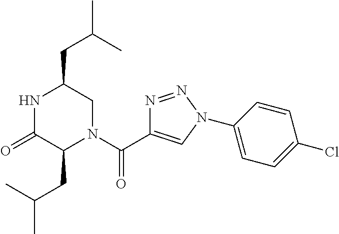 | 1.96 | | >10 |
| 351 | 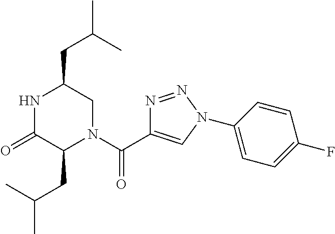 | 1.63 | | >10 |
| 352 | 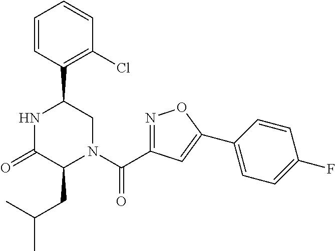 | 0.21 | | >10 |
| 354 | 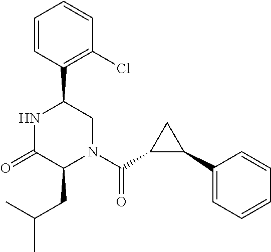 | 1.85 | | >10 |
| 355 | 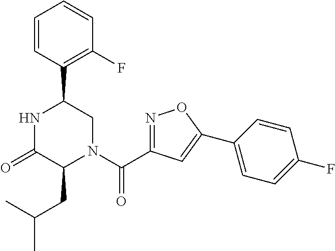 | 0.093 | 0.16 | >10 |
| 357 | 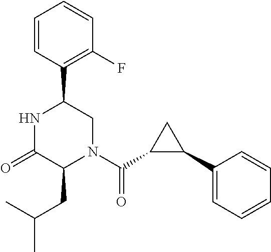 | 0.52 | | >10 |
| 358 | 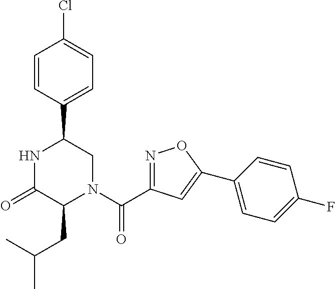 | 1.53 | | >10 |
| 360 | 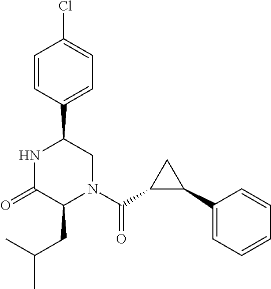 | 3.64 | | >10 |
| 361 | 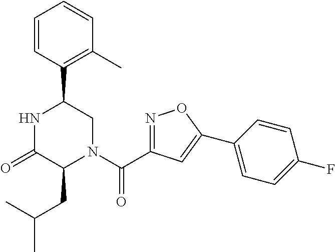 | 1.42 | | >10 |
| 363 | 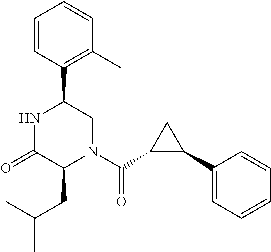 | 2.07 | | >10 |
| 364 | 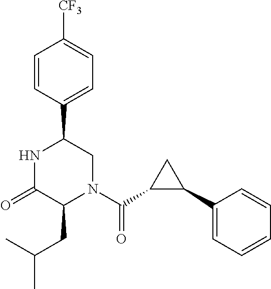 | >10 | | >10 |
| 366 | 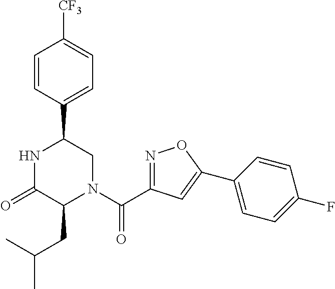 | 6.00 | | >10 |
| 367 | 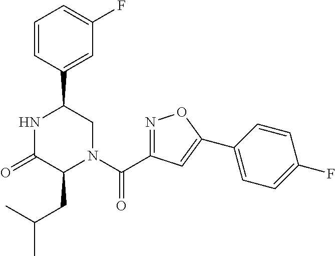 | 0.27 | | >10 |
| 369 | 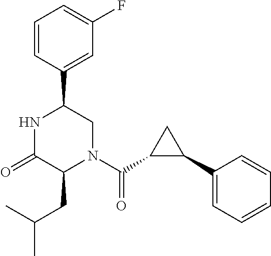 | 1.63 | | >10 |
| 370 | 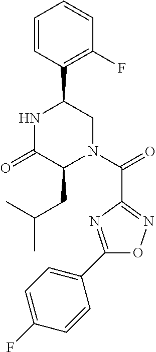 | 0.061 | 0.099 | >10 |
| 371 | 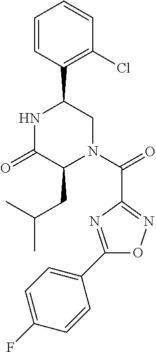 | 0.47 | | >10 |
| 372 | 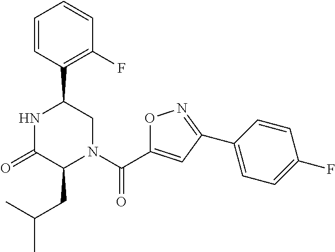 | 0.70 | | >10 |
| 373 | 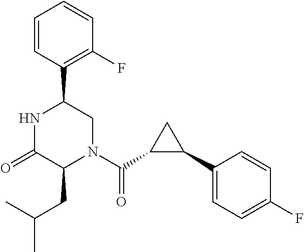 | 0.057 | 0.087 | >10 |
| 374 | 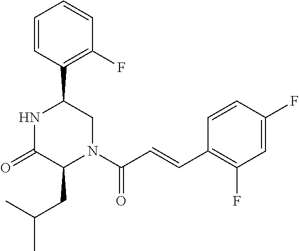 | 0.75 | | >10 |
| 375 | 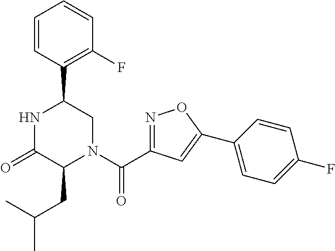 | 0.65 | | >10 |
| 376 | 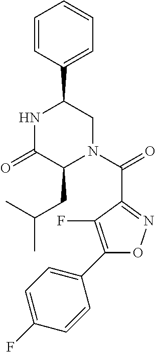 | 0.50 | | >10 |
| 379 | 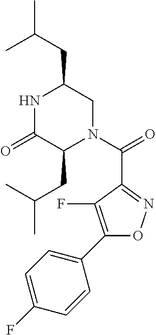 | 0.21 | | >10 |
| 380 | 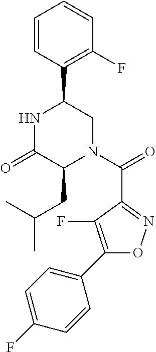 | 0.18 | | >10 |
| 381 | 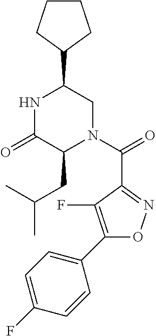 | 0.25 | | >10 |
| 382 | 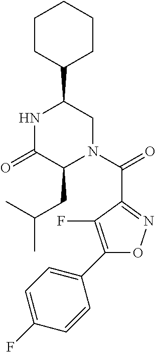 | 0.37 | | >10 |
| 383 | 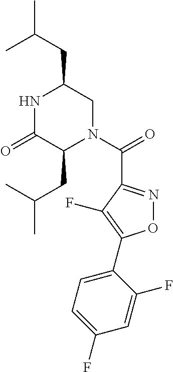 | 0.71 | | >10 |
| 385 | 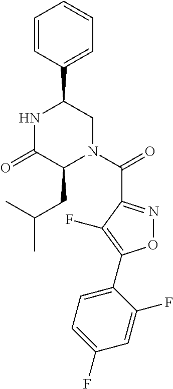 | 0.50 | | >10 |
| 386 | 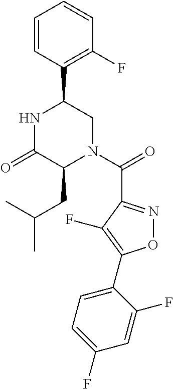 | 0.57 | | >10 |
| 387 | 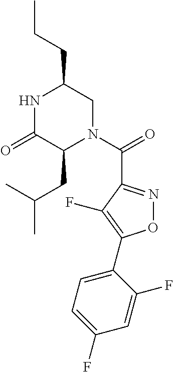 | 0.26 | | >10 |
| 388 | 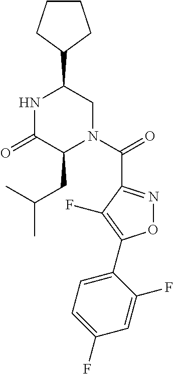 | 0.67 | | >10 |
| 389 | 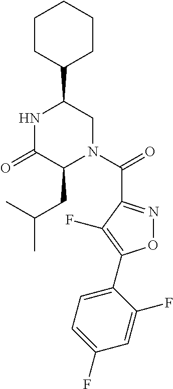 | 1.45 | | >10 |
| 390 | 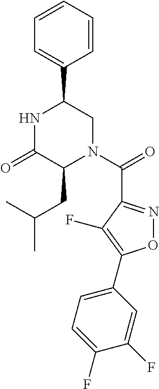 | 2.05 | | >10 |
| 392 | 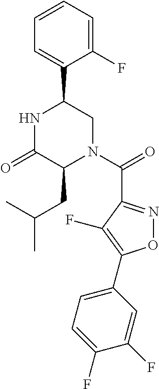 | 1.91 | | >10 |
| 393 | 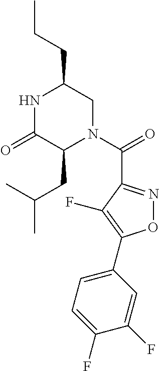 | 0.26 | | >10 |
| 394 | 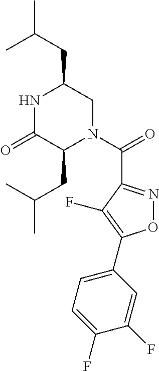 | 0.66 | | >10 |
| 395 | 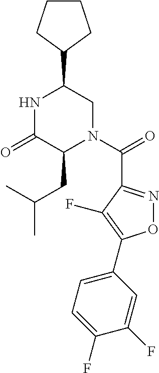 | 1.02 | | >10 |
| 396 | 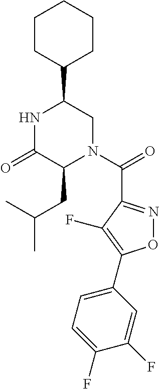 | 1.82 | | >10 |
| 397 | 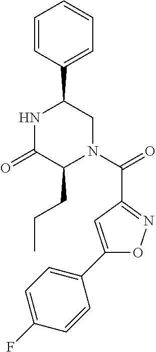 | 0.89 | | >10 |
| 399 | 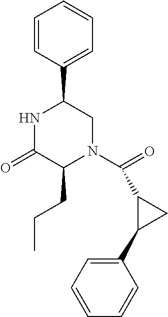 | 2.07 | | >10 |
| 400 | 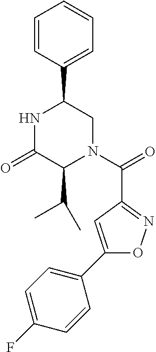 | 0.97 | | >10 |
| 402 | 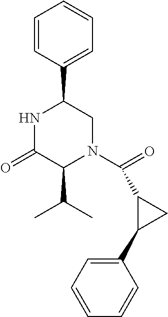 | 6.63 | | >10 |
| 403 | 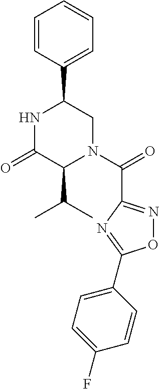 | 0.27 | | >10 |
| 404 | 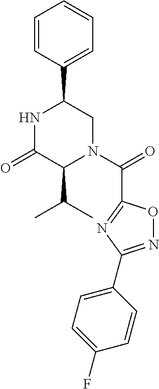 | 1.58 | | >10 |
| 405 | 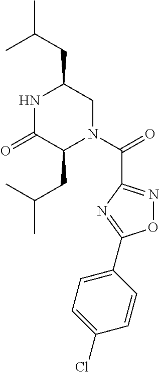 | 0.32 | | >10 |
| 406 | 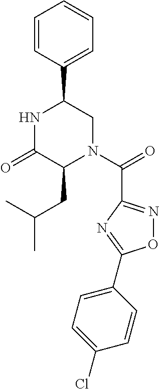 | 0.23 | | >10 |
| 407 | 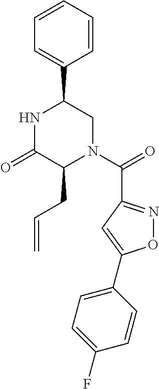 | 0.74 | | >10 |
| 409 | 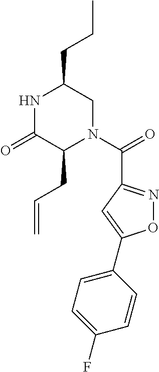 | 1.88 | | >10 |
| 411 | 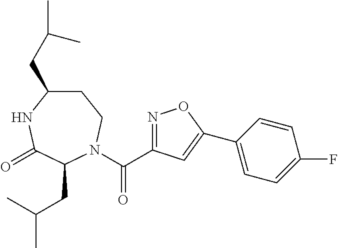 | >10 | | >10 |
| 413 | 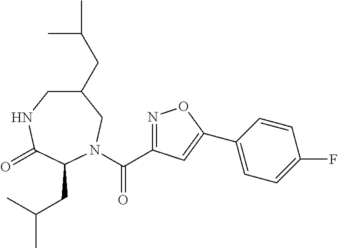 | >10 | | >10 |
| 415 | 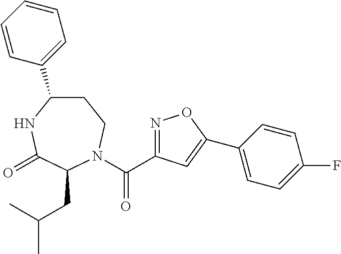 | >10 | | >10 |
| 417 | 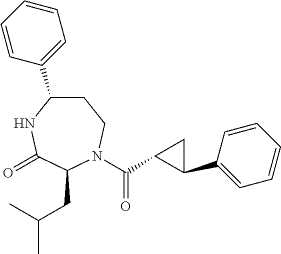 | 7.77 | | >10 |
| 418 | 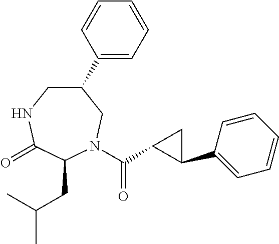 | >10 | | >10 |
| 420 | 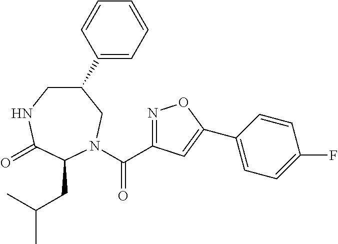 | >10 | | >10 |
| 421 | 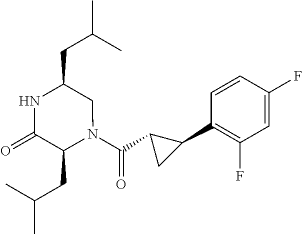 | >10 | | >10 |
| 422 | 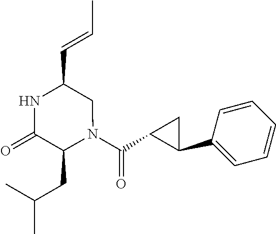 | 2.10 | | >10 |
| 424 | 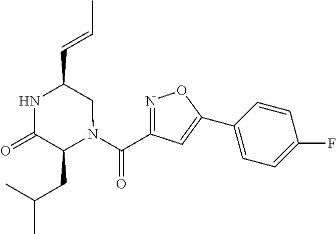 | 0.064 | 0.082 | >10 |
| 425 | 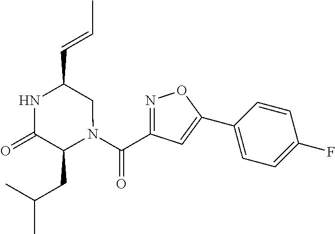 | 0.70 | | >10 |
| 426 | 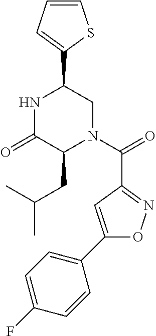 | 0.018 | 0.048 | >10 |
| 428 | 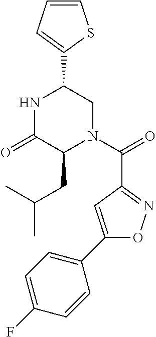 | >10 | | >10 |
| 430 | 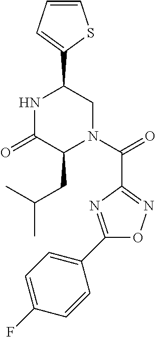 | 0.0026 | 0.018 | >10 |
| 431 | 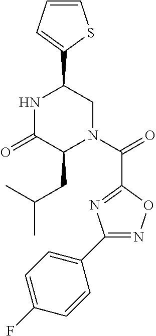 | 0.024 | | >10 |
| 432 | 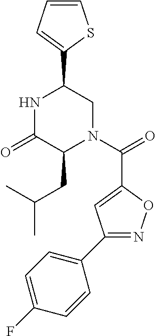 | 0.020 | 0.022 | >10 |
| 433 | 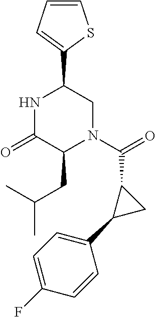 | 0.018 | 0.030 | >10 |
| 434 | 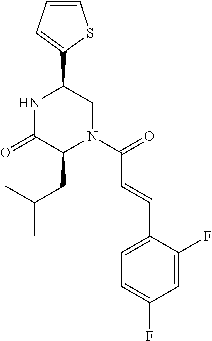 | 0.048 | | >10 |
| 435 | 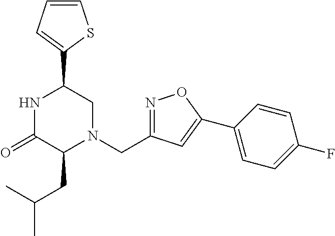 | 0.26 | | >10 |
| 436 | 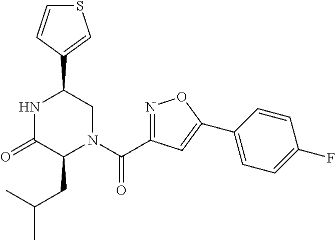 | 0.018 | 0.038 | >10 |
| 438 | 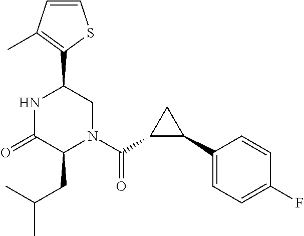 | >10 | | >10 |
| 441 | 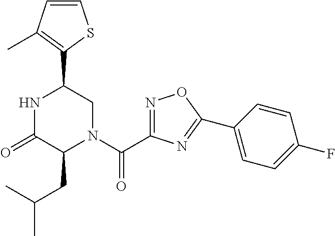 | >10 | | >10 |
| 442 | 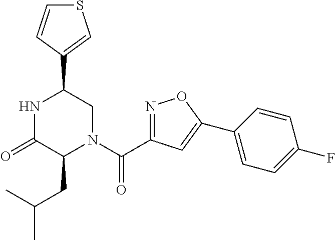 | 0.091 | | >10 |
| 443 | 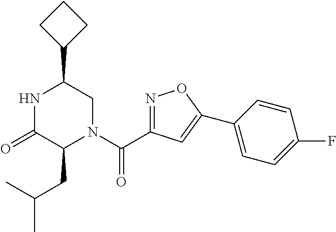 | 0.74 | | >10 |
| 445 | 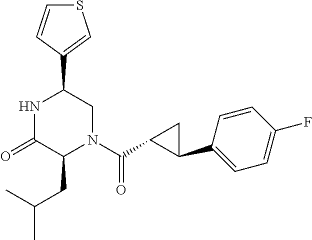 | 0.027 | 0.020 | >10 |
| 446 | 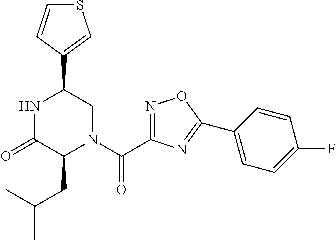 | 0.014 | 0.028 | >10 |
| 447 | 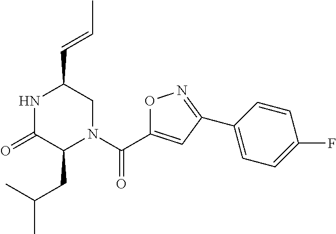 | 0.14 | | >10 |
| 448 | 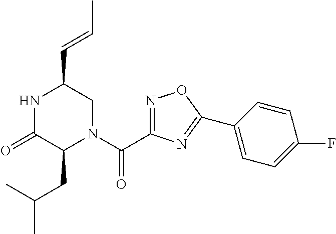 | 0.11 | | >10 |
| 449 | 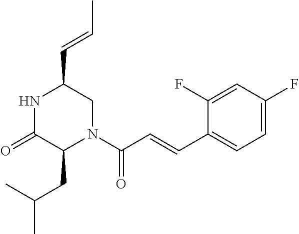 | 1.19 | | >10 |
| 450 | 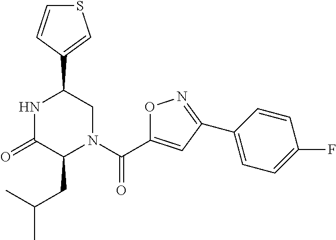 | 0.059 | | >10 |
| 451 | 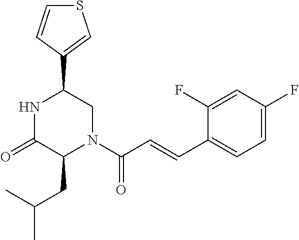 | 0.125 | | >10 |
| 452 | 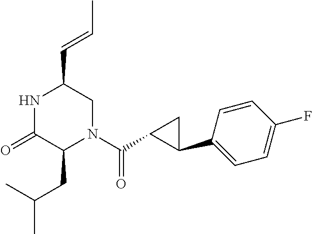 | 0.048 | | >10 |
| 453 | 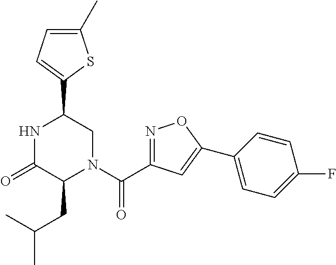 | 0.68 | | >10 |
| 456 | 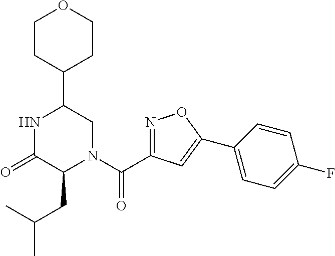 | 0.194 | | >10 |
| 458 | 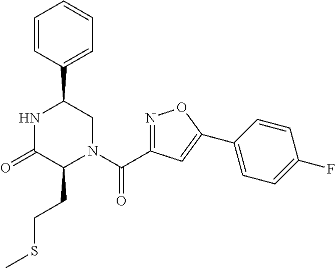 | 2.07 | | >10 |
| 460 | 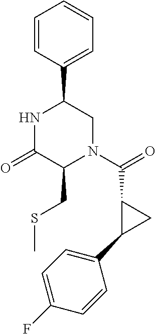 | 1.16 | | >10 |
| 462 | 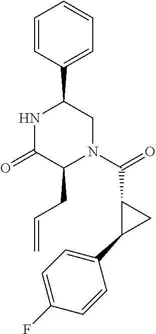 | 1.52 | | >10 |
| 464 | 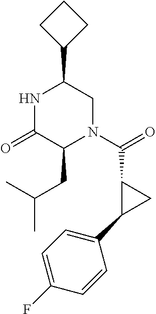 | 0.83 | | >10 |
| 465 | 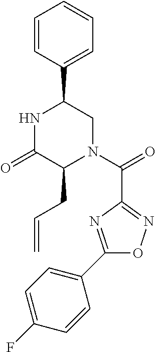 | 0.29 | | >10 |
| 466 | 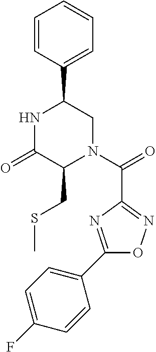 | 0.65 | | >10 |
| 467 | 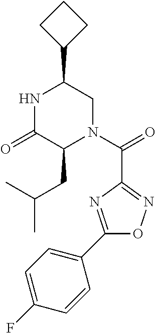 | 0.25 | | >10 |
| 468 | 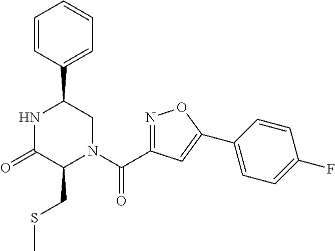 | 0.69 | | >10 |
| 469 | 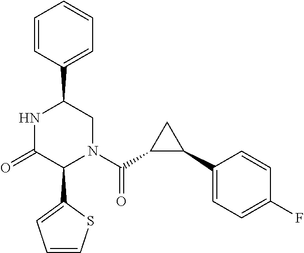 | 1.06 | | >10 |
| 471 | 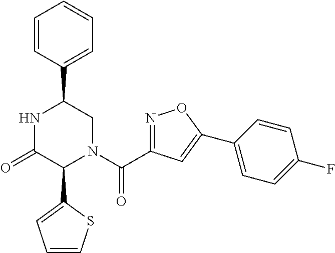 | 0.57 | | >10 |
| 472 | 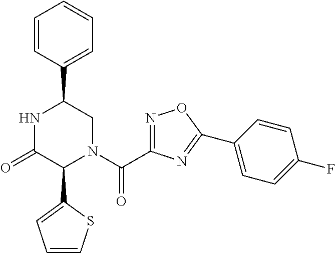 | 1.20 | | >10 |
| 473 | 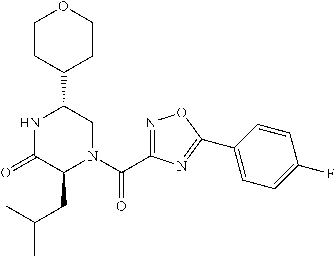 | >10 | | >10 |
| 474 | 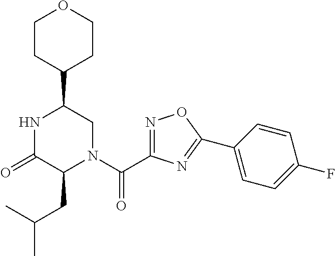 | 0.35 | | >10 |
| 475 | 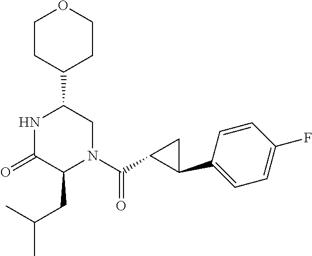 | >10 | | >10 |
| 476 | 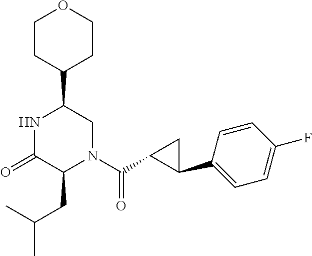 | 0.23 | | >10 |
| 483 | 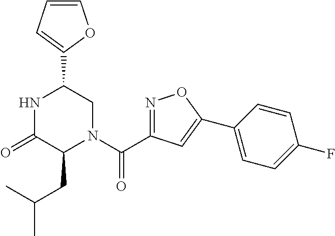 | >10 | | >10 |
| 484 | 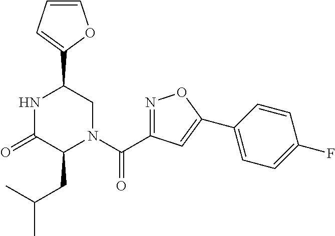 | 0.18 | | >10 |
| 485 | 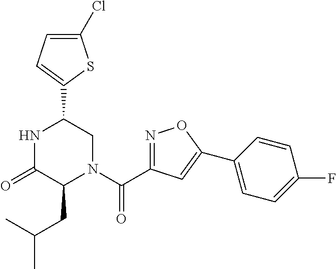 | >10 | | >10 |
| 488 | 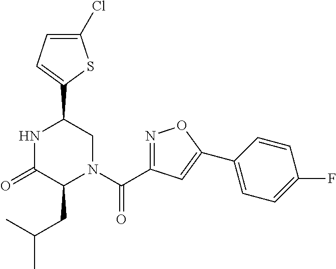 | 0.70 | | >10 |
| 489 | 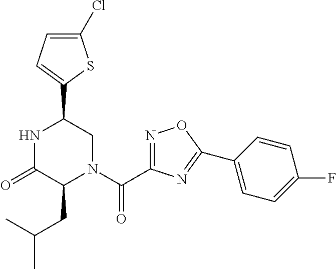 | 0.32 | | >10 |
| 490 | 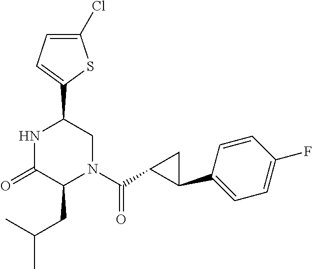 | 0.99 | | >10 |
| 491 | 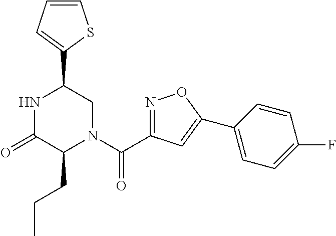 | 0.25 | | >10 |
| 494 | 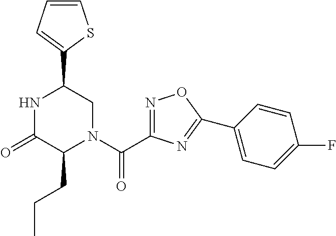 | 0.038 | | >10 |
| 495 | 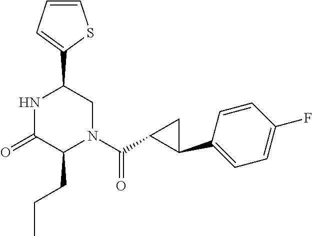 | 0.29 | | >10 |
| 496 | 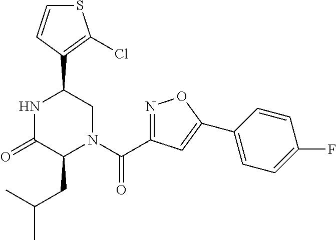 | 0.011 | 0.065 | >10 |
| 499 | 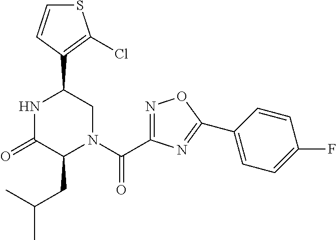 | 0.023 | 0.083 | >10 |
| 500 | 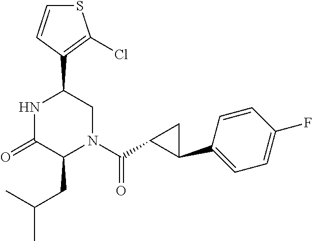 | 0.022 | | >10 |
| 506 | 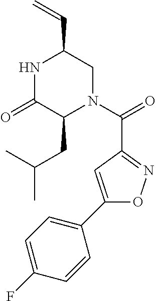 | 1.05 | | >10 |
| 507 | 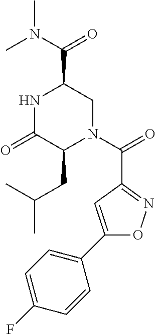 | 0.84 | | >10 |
| 508 | 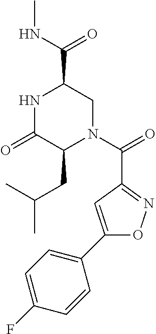 | >10 | | >10 |
| 510 | 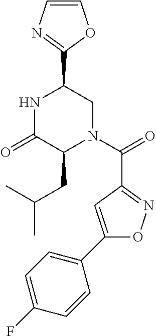 | >10 | | >10 |
| 511 | 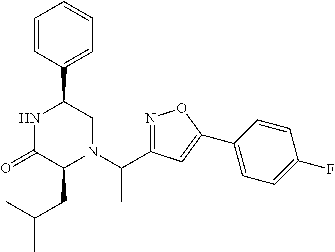 | 0.45 | | >10 |
| 516 | 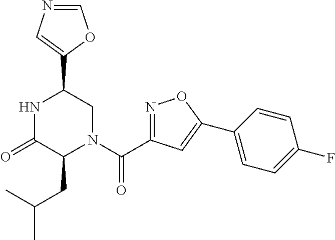 | 0.11 | | >10 |
| 517 | 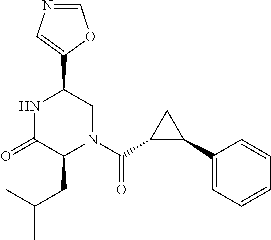 | 0.19 | | >10 |
| 518 | 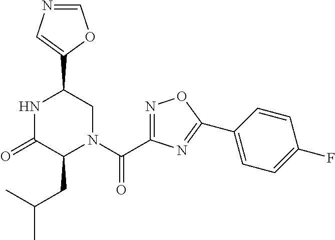 | 0.058 | | >10 |
| 519 | 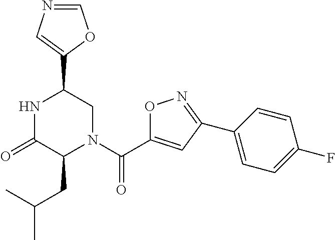 | 0.11 | | >10 |
| 520 | 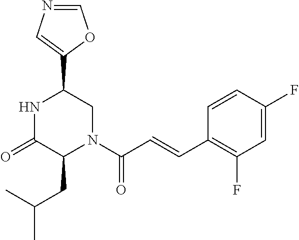 | 0.47 | | >10 |
| 521 | 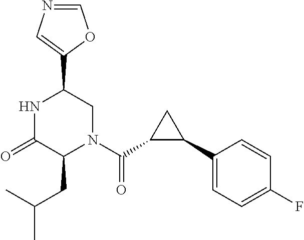 | 0.022 | 0.004 | >10 |
| 522 | 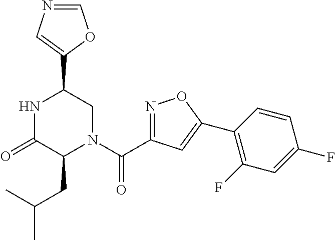 | 0.27 | | >10 |
| 523 | 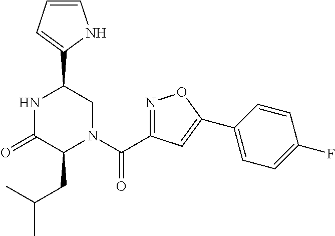 | 1.06 | | >10 |
| 524 | 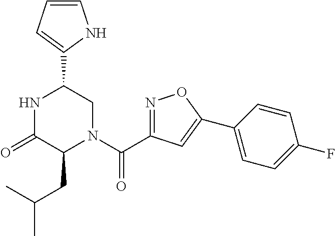 | >10 | | >10 |
| 528 | 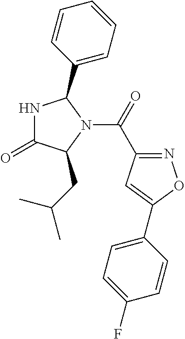 | >10 | | >10 |
| 529 | 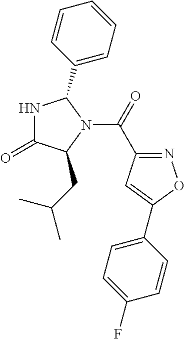 | >10 | | >10 |
| 530 | 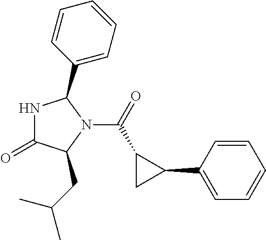 | >10 | | >10 |
| 531 | 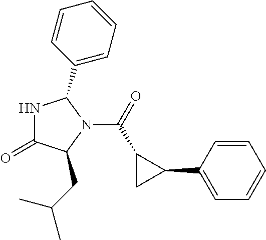 | >10 | | >10 |
| 532 | 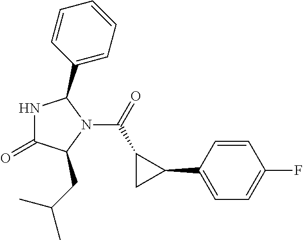 | >10 | | >10 |
| 533 | 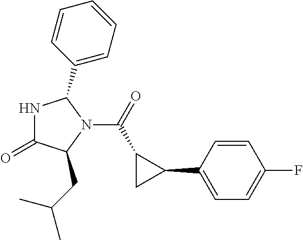 | >10 | | >10 |

The subject matter of U.S. 61/435,528, filed Jan. 24, 2011, is hereby incorporated by its reference in its entirety. It is understood that the meanings of the subject matter incorporated from U.S. 61/435,528 are subservient to the meanings of the subject matter disclosed herein.

Although a full and complete description is believed to be contained herein, certain patent and non-patent references may include certain essential subject matter. To the extent that these patent and non-patent references describe essential subject matter, these references are hereby incorporated by reference in their entirety. It is understood that the meanings of the incorporated subject matter are subservient to the meanings of the subject matter disclosed herein.

1. A compound or its stereoisomer or its salt thereof represented by the following formula A: 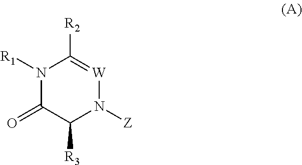
wherein
C [symbol] W is a single-bond or a double-bond, where
(i) W is CH, when C [symbol] W is a double-bond,
(ii) W is CH₂, CHR₄, or CHR₅CHR₆ when C [symbol] W is a single-bond,
or
(iii) W is absent and the carbon atom of ˜C(—R₂)˜ is bound directly to the nitrogen atom of ˜N(—Z)˜ to form a compound having the structure A′ 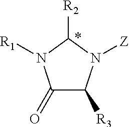
R₁ is selected from among hydrogen, an alkyl, an alkaryl, an acyl, an aryl, and a heteroaryl;
R₂, R₃, R₄, R₅, and R₆ are independently selected from among, hydrogen, an alkyl, an allyl, an alkaryl, halo, a cycloalkyl, an alkylene-cycloalkyl, an alkylenethioalkyl, an alkyleneoxoalkyl, an alkylene-cycloalkyl, an aryl, an alkaryl, a heteroaryl, an alkheteroaryl, COOH, COOalkyl, CONHalkyl, and CON(alkyl)₂; and
Z is ˜Q-Yₘ—B or ˜T-A-Xₙ—B
where
Q is —(CH₂)ₒ— (o is 0, 1, 2, or 3) or —C(O)—;
Y is O, S, or NH and m is 0 or 1;
T is —(CH₂)ₚ—(C(O))_{q}— (p is 0, 1, 2, or 3 and q is 0 or 1) or CHR₇—C(O)—, where R₇ is a C₁₋₆alkyl;
X is CH₂, O, NH, or S, with n is 0 or 1;
A is selected from among —CR′═CR″—, —C≡C—, a cycloalkylene, an arylene, and a heteroarylene,
where R′ and R″ are independently selected from among hydrogen, a C₁₋₆alkyl, and an aryl; and
B is selected from among hydrogen, a C₁₋₆alkyl, a cycloalkyl, an aryl, an aryloxide, a heteroaryl, and a fused ring moiety.

2. A composition comprising the compound or its stereoisomer or its salt thereof according to claim 1 and a pharmaceutically acceptable medium.

3. A composition for treating a hepatitis C virus, which comprises an effective amount of comprising the compound or its stereoisomer or its salt thereof according to claim 1 and a pharmaceutically acceptable medium.

4. A method of treating a subject infected by a virus, which comprises:
administering to the subject an effective amount of comprising the compound or its stereoisomer or its salt thereof according to claim 1;
wherein the virus is selected from among hepatiis C virus, West Nile virus, a yellow fever virus, a dengue virus, a rhinovirus, a polio virus, a hepatitis A virus, a bovine viral diarrhea virus, and a Japanese encephalitis virus.

5. A method of treating a subject infected by hepatitis C virus, which comprises:
administering to the subject an effective amount of the compound or its stereoisomer or its salt thereof according to claim 1.

6. A method of treating a subject infected by hepatitis C virus, which comprises:
administering to the subject an effective amount of the compound or its stereoisomer or its salt thereof according to claim 1 and
administering to the subject an effective amount of another antiviral agent.

7. A use of comprising the compound or its stereoisomer or its salt thereof according to claim 1 for the manufacture of a medicament for the treatment of a condition that results from an infection by hepatitis C virus, West Nile virus, yellow fever virus, dengue virus, rhinovirus, polio virus, hepatitis A virus, bovine viral diarrhea virus or Japanese encephalitis virus.

8. A use of comprising the compound or its stereoisomer or its salt thereof according to claim 1 for the manufacture of a medicament for the treatment of a condition that results from an infection by hepatitis C virus.

9. A process for preparing comprising the compound or its stereoisomer or its salt thereof according to claim 1 as disclosed by any of the procedures disclosed herein.

10. A product comprising the compound or its stereoisomer or its salt thereof according to claim 1 obtained by a process as disclosed by any of the procedures disclosed herein.

11. A compound or salt thereof selected from among
(3S,6S)-4-(4-fluoro-2-(trifluoromethyl)benzyl)-3,6-diisobutylpiperazin-2-one (220); (3S,6S)-4-(4-fluoro-3-(trifluoromethyl)benzyl)-3,6-diisobutylpiperazin-2-one (221); (3S,6S)-4-(2,5-difluorobenzyl)-3,6-diisobutylpiperazin-2-one (222); (3S,6S)-4-(3,5-Bis-trifluoromethyl-benzyl)-3,6-diisobutyl-piperazin-2-one (223); (3S,6S)-4-(2,6-Difluoro-benzyl)-3,6-diisobutyl-piperazin-2-one (224); (3S,6S)-4-(4-Ethyl-benzyl)-3,6-diisobutyl-piperazin-2-one (225); (3S,6S)-4-(3,4-Dichloro-benzyl)-3,6-diisobutyl-piperazin-2-one (226); (3S,6S)-4-(3-Chloro-4-fluoro-benzyl)-3,6-diisobutyl-piperazin-2-one (227); (3S,6S)-3,6-Diisobutyl-4-(4-trifluoromethoxy-benzyl)-piperazin-2-one (228); (3S,6S)-4-(2-Chloro-5-trifluoromethyl-benzyl)-3,6-diisobutyl-piperazin-2-one (229); (3S,6S)-3,6-Diisobutyl-4-(3-phenyl-allyl)-piperazin-2-one (77); (3S,6S)-4-(1-(5-(4-fluorophenyl)isoxazol-3-yl)ethyl)-3-isobutyl-6-phenylpiperazin-2-one (511); (3S,6S)-3,6-Diisobutyl-4-[(E)-(3-phenyl-acryloyl)]-piperazin-2-one (71); (3S,6S)-4-[3-(4-Chloro-phenyl)-acryloyl]-3,6-diisobutyl-piperazin-2-one (72); (3S,6S)-3,6-Diisobutyl-4-[3-(4-trifluoromethyl-phenyl)-acryloyl]-piperazin-2-one (73); (3S,6S)-3-Isobutyl-6-methyl-4-(3-pyridin-3-yl-acryloyl)-piperazin-2-one (74); (3S,6S)-3,6-Diisobutyl-4-(3-phenyl-but-2-enoyl)-piperazin-2-one (75); (3S,6S)-3,6-Diisobutyl-4-(2-methyl-3-phenyl-acryloyl)-piperazin-2-one (78); (3S,6S)-3,6-Diisobutyl-4-(3-p-tolyl-acryloyl)-piperazin-2-one (79); (3S,6S)-4-[3-(4-Fluoro-phenyl)-acryloyl]-3,6-diisobutyl-piperazin-2-one (80); (3S,6S)-4-[3-(3,4-Dichloro-phenyl)-acryloyl]-3,6-diisobutyl-piperazin-2-one (81); (3S,6S)-4-[3-(3,4-Difluoro-phenyl)-acryloyl]-3,6-diisobutyl-piperazin-2-one (82); (3S,6S)-4-[3-(3-Fluoro-phenyl)-acryloyl]-3,6-diisobutyl-piperazin-2-one (83); (3S,6S)-3,6-Diisobutyl-4-[3-(3,4,5-trifluoro-phenyl)-acryloyl]-piperazin-2-one (84); (3S,6S)-4-[3-(3-Chloro-4-fluoro-phenyl)-acryloyl]-3,6-diisobutyl-piperazin-2-one (85); (3S,6S)-4-[3-(4-Chloro-2-fluoro-phenyl)-acryloyl]-3,6-diisobutyl-piperazin-2-one (86); (3S,6S)-4-[3-(2,4-Difluoro-phenyl)-acryloyl]-3,6-diisobutyl-piperazin-2-one (87); (3S,6S)-4-((E)-3-(Benzo[d][1,3]dioxol-5-yl)acryloyl)-3,6-diisobutylpiperazin-2-one (90); (3S,6S)-3,6-Diisobutyl-4-((E)-3-(4-nitrophenyl)acryloyl)piperazin-2-one (91); (3S,6S)-3,6-Diisobutyl-4-((E)-3-(4-(methylsulfonyl)phenyl)acryloyl)piperazin-2-one (92); (3S,6S)-4-((E)-3-(2-Fluoro-4-(trifluoromethyl)phenyl)acryloyl)-3,6-diisobutylpiperazin-2-one (93); (3S,6S)-4-((E)-3-([1,1′-Biphenyl]-4-yl)acryloyl)-3,6-diisobutylpiperazin-2-one (94); (3S,6S)-4-((E)-3-(3,5-Difluorophenyl)acryloyl)-3,6-diisobutylpiperazin-2-one (95); (3S,6S)-4-((E)-3-(2,4-difluorophenyl)acryloyl)-3-isobutyl-6-propylpiperazin-2-one (96); (3S,6S)-4-((E)-3-(4-(dimethylamino)phenyl)acryloyl)-3,6-diisobutylpiperazin-2-one (261); (3S,6S)-3,6-diisobutyl-4-((E)-3-(4-methoxyphenyl)acryloyl)piperazin-2-one (263); (3S,6S)-4-[3-(3-chlorophenyl)-acryloyl]-3,6-diisobutyl-piperazin-2-one (264); (3S,6S)-4-[3-(2-chlorophenyl)-acryloyl]-3,6-diisobutyl-piperazin-2-one (265); (3S,6S)-4-[3-(2-chlorophenyl)-acryloyl]-3,6-diisobutyl-piperazin-2-one (266); (3S,6S)-4-[3-(2,4-dichloro-phenyl)-acryloyl]-3,6-diisobutyl-piperazin-2-one (267); (3S,6S)-3,6-diisobutyl-4-((E)-3-(4-methylsulfanyl-phenyl)acryloyl)piperazin-2-one (268); (3S,6S)-3,6-diisobutyl-4-((E)-3-(4-tert-butyl-phenyl)acryloyl)piperazin-2-one (269); Methyl 4-((E)-3-((2S,5S)-2,5-diisobutyl-3-oxopiperazin-1-yl)-3-oxoprop-1-en-1-yl)-benzoate (270); (3S,6S)-4-[3-(2,6-Difluoro-phenyl)-acryloyl]-3,6-diisobutyl-piperazin-2-one (271); (3S,6S)-4-((E)-3-(2,4-difluorophenyl)acryloyl)-6-(2-fluorophenyl)-3-isobutylpipera-zin-2-one (374); (3S,6R)-4-((E)-3-(2,4-difluorophenyl)acryloyl)-3-isobutyl-6-(thiophen-2-yl)pipera-zin-2-one (434); (3S,6S)-4-((E)-3-(2,4-difluorophenyl)acryloyl)-3-isobutyl-6-((E)-prop-1-en-1-yl)piperazin-2-one (449); (3S,6S)-4-((E)-3-(2,4-difluorophenyl)acryloyl)-3-isobutyl-6-(thiophen-3-yl)piperazin-2-one (451); (3S,6R)-4-((E)-3-(2,4-difluorophenyl)acryloyl)-3-isobutyl-6-(oxazol-5-yl)piperazin-2-one (520); (3S,6S)-3,6-Diisobutyl-4-(5-phenylfuran-2-carbonyl)piperazin-2-one (97); (3S,6S)-3,6-Diisobutyl-4-(2-phenylthiazole-4-carbonyl)piperazin-2-one (98); (3S,6S)-3,6-Diisobutyl-4-(2-phenyloxazole-4-carbonyl)piperazin-2-one (99); (3S,6S)-3,6-Diisobutyl-4-(5-phenyl-1H-pyrazole-3-carbonyl)piperazin-2-one (100); (3S,6S)-3,6-Diisobutyl-4-(2-phenyl-1H-imidazole-4-carbonyl)piperazin-2-one (101); (3S,6S)-3,6-Diisobutyl-4-(1-phenyl-1H-imidazole-4-carbonyl)piperazin-2-one (102); (3S,6S)-4-(1-(4-Fluorophenyl)-1H-imidazole-4-carbonyl)-3,6-diisobutylpiperazin-2-one (103); (3S,6S)-4-(1-(4-Fluorophenyl)-1H-pyrazole-4-carbonyl)-3,6-diisobutylpiperazin-2-one (104); (3S,6S)-4-(3-(4-Chlorophenyl)isothiazole-5-carbonyl)-3,6-diisobutylpiperazin-2-one (105); (3S,6S)-4-(5-Cyclopropylisoxazole-3-carbonyl)-3,6-diisobutylpiperazin-2-one (106); (3S,6S)-4-(5-Ethylisoxazole-3-carbonyl)-3,6-diisobutylpiperazin-2-one (107); (3S,6S)-3,6-Diisobutyl-4-(5-phenylisoxazole-3-carbonyl)piperazin-2-one (108); (3S,6S)-3,6-Diisobutyl-4-(5-(thiophen-2-yl)isoxazole-3-carbonyl)piperazin-2-one (109); (3S,6S)-3-Isobutyl-6-phenyl-4-(5-(thiophen-2-yl)isoxazole-3-carbonyl)piperazin-2-one (110); (3S,6S)-3-Isobutyl-6-isopropyl-4-(5-(thiophen-2-yl)isoxazole-3-carbonyl)piperazin-2-one (111); (3S,6S)-4-(5-(5-Chlorothiophen-2-yl)isoxazole-3-carbonyl)-3,6-diisobutylpiperazin-2-one (112); (3S,6S)-4-(5-(3,4-Difluorophenyl)isoxazole-3-carbonyl)-3,6-diisobutylpiperazin-2-one (123); (3S,6S)-4-(5-(4-Fluorophenyl)isoxazole-3-carbonyl)-3,6-diisobutylpiperazin-2-one (124); (3S,6S)-4-(5-(4-Chlorophenyl)isoxazole-3-carbonyl)-3,6-diisobutylpiperazin-2-one (125); (3S,6S)-3,6-Diisobutyl-4-(5-(p-tolyl)isoxazole-3-carbonyl)piperazin-2-one (126); (3S,6S)-3,6-Diisobutyl-4-(5-(4-methoxyphenyl)isoxazole-3-carbonyl)piperazin-2-one (127); (3S,6S)-4-(5-(3-Fluorophenyl)isoxazole-3-carbonyl)-3,6-diisobutylpiperazin-2-one (128); (3S,6S)-4-(5-(3-Chlorophenyl)isoxazole-3-carbonyl)-3,6-diisobutylpiperazin-2-one (129); (3S,6S)-3,6-Diisobutyl-4-(5-(3-methoxyphenyl)isoxazole-3-carbonyl)piperazin-2-one (130); (3S,6S)-3,6-diisobutyl-4-(5-(2-methoxyphenyl)isoxazole-3-carbonyl)piperazin-2-one (131); (3S,6S)-4-(5-(4-(tert-butyl)phenyl)isoxazole-3-carbonyl)-3,6-diisobutylpiperazin-2-one (132); (3S,6S)-4-(5-(2,4-difluorophenyl)isoxazole-3-carbonyl)-3,6-diisobutylpiperazin-2-one (133); (3S,6S)-4-(5-(4-chloro-3-fluorophenyl)isoxazole-3-carbonyl)-3,6-diisobutylpiperazin-2-one (134); (3S,6S)-4-(5-(4-Bromophenyl)isoxazole-3-carbonyl)-3,6-diisobutylpiperazin-2-one (135); 4-(3-((2S,5S)-2,5-Diisobutyl-3-oxopiperazine-1-carbonyl)isoxazol-5-yl)benzonitrile (136); 4-(3-((2S,5S)-5-Cyclopentyl-2-isobutyl-3-oxopiperazine-1-carbonyl)isoxazol-5-yl)benzonitrile (137); (3S,6S)-3,6-Diisobutyl-4-(5-(3-(trifluoromethoxy)phenyl)isoxazole-3-carbonyl)piperazin-2-one (138); 4-(3-((2S,5S)-2,5-Diisobutyl-3-oxopiperazine-1-carbonyl)isoxazol-5-yl)-N,N-dimethylbenzamide (139); (3S,6S)-4-(5-(4-(Dimethylamino)phenyl)isoxazole-3-carbonyl)-3,6-diisobutylpiperazin-2-one (140); (3S,6S)-4-(5-(3-Chloro-4-fluorophenyl)isoxazole-3-carbonyl)-3,6-diisobutylpiperazin-2-one (141); (3S,6S)-4-(5-(2-Fluorophenyl)isoxazole-3-carbonyl)-3,6-diisobutylpiperazin-2-one (142); (3S,6S)-4-(5-(4-Chloro-2-fluorophenyl)isoxazole-3-carbonyl)-3,6-diisobutylpiperazin-2-one (143); (3S,6S)-4-(3-(4-Fluorophenyl)isoxazole-5-carbonyl)-3,6-diisobutylpiperazin-2-one (144); (3S,6S)-4-(3-(4-Fluorophenyl)isoxazole-5-carbonyl)-3-isobutyl-6-phenylpiperazin-2-one (145); (3S,6S)-4-(3-(4-Fluorophenyl)isoxazole-5-carbonyl)-3-isobutyl-6-isopropylpiperazin-2-one (146); (3S,6R)-4-(3-(4-Fluorophenyl)isoxazole-5-carbonyl)-3-isobutyl-6-((methylthio)methyl)piperazin-2-one (147); (3S,6S)-4-(3-(4-Fluorophenyl)-1,2,4-oxadiazole-5-carbonyl)-3,6-diisobutylpiperazin-2-one (148); (3S,6S)-4-(3-(4-Fluorophenyl)-1,2,4-oxadiazole-5-carbonyl)-3-isobutyl-6-propylpiperazin-2-one (149); (3S,6S)-3-Isobutyl-4-(3-phenyl-1,2,4-oxadiazole-5-carbonyl)-6-propylpiperazin-2-one (150); (3S,6S)-4-(3-(4-Fluorophenyl)-1,2,4-oxadiazole-5-carbonyl)-3-isobutyl-6-phenylpiperazin-2-one (151); (3S,6S)-6-Cyclopentyl-4-(3-(4-fluorophenyl)-1,2,4-oxadiazole-5-carbonyl)-3-isobutylpiperazin-2-one (152); (3S,6S)-4-(3-(4-Fluorophenyl)-1,2,4-oxadiazole-5-carbonyl)-3-isobutyl-6-isopropylpiperazin-2-one (153); (3S,6R)-4-(3-(4-Fluorophenyl)-1,2,4-oxadiazole-5-carbonyl)-3-isobutyl-6-((methylthio)methyl)piperazin-2-one (154); (3S,6S)-3,6-Diisobutyl-4-[5-(4-nitro-phenyl)-isoxazole-3-carbonyl]-piperazin-2-one (155); (3S,6S)-6-Cyclohexyl-4-[5-(2,4-difluoro-phenyl)-isoxazole-3-carbonyl]-3-isobutyl-piperazin-2-one (156); (3S,6S)-6-Cyclopentyl-4-[5-(2,4-difluoro-phenyl)-isoxazole-3-carbonyl]-3-isobutyl-piperazin-2-one (157); (3S,6S)-4-[5-(4-Chloro-3-fluoro-phenyl)-isoxazole-3-carbonyl]-3-isobutyl-6-propyl-piperazin-2-one (158); (3S,6S)-4-[5-(4-Chloro-3-fluoro-phenyl)-isoxazole-3-carbonyl]-6-cyclohexyl-3-isobutyl-piperazin-2-one (159); (3S,6S)-4-[(5-(4-Chloro-3-fluoro-phenyl)-isoxazole-3-carbonyl]-6-cyclopentyl-3-isobutyl-piperazin-2-one (160); (3S,6S)-4-(5-(4-fluorophenyl)isoxazole-3-carbonyl)-3-isobutyl-6-propylpiperazin-2-one (161); (3S,6S)-6-cyclohexyl-4-(5-(4-fluorophenyl)isoxazole-3-carbonyl)-3-isobutylpiperazin-2-one (162); (3S,6S)-6-cyclopentyl-4-(5-(4-fluorophenyl)isoxazole-3-carbonyl)-3-isobutyl-piperazin-2-one (163); (3S,6S)-4-(5-(2,4-difluorophenyl)isoxazole-3-carbonyl)-3,6-diisobutylpiperazin-2-one (164); (3S,6S)-4-(5-(2,4-difluorophenyl)isoxazole-3-carbonyl)-3-isobutyl-6-propylpiperazin-2-one (165); (3S,6S)-4-(5-(4-chlorophenyl)isoxazole-3-carbonyl)-6-(cyclopropylmethyl)-3-isobutylpiperazin-2-one (166); (3S,6S)-4-(5-(4-fluorophenyl)isoxazole-3-carbonyl)-6-(cyclopropylmethyl)-3-isobutylpiperazin-2-one (167); (3S,6S)-6-(cyclopropylmethyl)-3-isobutyl-4-(5-(thiophen-2-yl)isoxazole-3-carbonyl)piperazin-2-one (168); (3S,6S)-3-isobutyl-6-propyl-4-(5-(thiophen-2-yl)isoxazole-3-carbonyl)piperazin-2-one (169); (3S,6S)-4-(5-(4-chlorophenyl)isoxazole-3-carbonyl)-3-isobutyl-6-propylpiperazin-2-one (170); (3S,6S)-4-(5-(4-chlorophenyl)isoxazole-3-carbonyl)-6-cyclohexyl-3-isobutylpiperazin-2-one (171); (3S,6S)-6-cyclohexyl-3-isobutyl-4-(5-(thiophen-2-yl)isoxazole-3-carbonyl)piperazin-2-one (172); (3S,6S)-4-(5-(4-chlorophenyl)isoxazole-3-carbonyl)-6-cyclopentyl-3-isobutylpiperazin-2-one (173); (3S,6S)-6-cyclopentyl-3-isobutyl-4-(5-(thiophen-2-yl)isoxazole-3-carbonyl)piperazin-2-one (174); (3S,6S)-4-(5-(4-fluorophenyl)isoxazole-3-carbonyl)-3-isobutyl-6-isopropylpiperazin-2-one (175); (3S,6S)-3,6-diisobutyl-4-(5-(4-(methylsulfonyl)phenyl)isoxazole-3-carbonyl)piperazin-2-one (176); (3S,6S)-4-(5-(4-chloro-3-fluorophenyl)isoxazole-3-carbonyl)-3,6-diisobutylpiperazin-2-one (177); (3S,6S)-4-(5-(4-fluorophenyl)isoxazole-3-carbonyl)-3-isobutyl-6-phenylpiperazin-2-one (178); (3S,6S)-3,6-diisobutyl-4-(5-(4-(trifluoromethoxy)phenyl)isoxazole-3-carbonyl)piperazin-2-one (179); (3S,6S)-6-cyclopropyl-4-(5-(4-fluorophenyl)isoxazole-3-carbonyl)-3-isobutylpiperazin-2-one (180); (3S,6S)-4-(5-(4-chlorophenyl)isoxazole-3-carbonyl)-3-isobutyl-6-isopropylpiperazin-2-one (181); (3S,6R)-4-(5-(4-chlorophenyl)isoxazole-3-carbonyl)-3-isobutyl-6-((methylthio)methyl)piperazin-2-one (182); (3S,6S)-4-(5-(4-chlorophenyl)isoxazole-3-carbonyl)-3-isobutyl-6-phenylpiperazin-2-one (183); (3S,6S)-4-(5-(3,4-difluorophenyl)isoxazole-3-carbonyl)-3-isobutyl-6-propylpiperazin-2-one (184); (3S,6S)-6-cyclohexyl-4-(5-(3,4-difluorophenyl)isoxazole-3-carbonyl)-3-isobutylpiperazin-2-one (185); (3S,6S)-6-cyclopentyl-4-(5-(3,4-difluorophenyl)isoxazole-3-carbonyl)-3-isobutylpiperazin-2-one (186); (3S,6S)-4-(5-(3,4-difluorophenyl)isoxazole-3-carbonyl)-3-isobutyl-6-isopropylpiperazin-2-one (187); (3S,6S)-4-(5-(3,4-difluorophenyl)isoxazole-3-carbonyl)-3-isobutyl-6-phenylpiperazin-2-one (188); (3S,6S)-4-(3-(4-fluorophenyl)isoxazole-5-carbonyl)-3-isobutyl-6-propylpiperazin-2-one (189); (3S,6S)-4-[3-(4-Chloro-phenyl)-isoxazole-5-carbonyl]-3,6-diisobutyl-piperazin-2-one (190); (3S,6S)-6-(cyclopropylmethyl)-4-(3-(4-fluorophenyl)isoxazole-5-carbonyl)-3-isobutylpiperazin-2-one (191); (3S,6S)-4-(3-(4-fluorophenyl)isoxazole-5-carbonyl)-3-isobutyl-6-propylpiperazin-2-one (192); (3S,6S)-6-cyclohexyl-4-(3-(4-fluorophenyl)isoxazole-5-carbonyl)-3-isobutylpiperazin-2-one (193); (3S,6S)-6-cyclopentyl-4-(3-(4-fluorophenyl)isoxazole-5-carbonyl)-3-isobutylpiperazin-2-one (194); (3S,6S)-3,6-Diisobutyl-4-((5-phenylisoxazol-3-yl)methyl)piperazin-2-one (195); (3S,6S)-4-((5-(4-Fluorophenyl)isoxazol-3-yl)methyl)-3,6-diisobutylpiperazin-2-one (196); (3S,6S)-4-((5-(4-Chlorophenyl)isoxazol-3-yl)methyl)-3,6-diisobutylpiperazin-2-one (197); (3S,6S)-4-((5-(4-Bromophenyl)isoxazol-3-yl)methyl)-3,6-diisobutylpiperazin-2-one (198); (3S,6S)-3,6-Diisobutyl-4-((5-(p-tolyl)isoxazol-3-yl)methyl)piperazin-2-one (199); (3S,6S)-3,6-Diisobutyl-4-((5-(4-methoxyphenyl)isoxazol-3-yl)methyl)piperazin-2-one (200); (3S,6S)-4-((5-(Furan-2-yl)isoxazol-3-yl)methyl)-3,6-diisobutylpiperazin-2-one (201); (3S,6S)-3,6-Diisobutyl-4-((5-(thiophen-2-yl)isoxazol-3-yl)methyl)piperazin-2-one (202); (3S,6S)-6-Cyclopropylmethyl-4-[5-(4-fluoro-phenyl)-isoxazol-3-ylmethyl]-3-isobutyl-piperazin-2-one (203); (3S,6S)-6-(2,2-Dimethyl-propyl)-4-[5-(4-fluoro-phenyl)-isoxazol-3-ylmethyl]-3-isobutyl-piperazin-2-one (204); (3S,6S)-6-(tert-butyl)-4-((5-(4-fluorophenyl)isoxazol-3-yl)methyl)-3-isobutylpiperazin-2-one (205); (3S,6S)-4-((5-(4-fluorophenyl)isoxazol-3-yl)methyl)-3-isobutyl-6-propylpiperazin-2-one (206); (3S,6S)-6-cyclohexyl-4-((5-(4-fluorophenyl)isoxazol-3-yl)methyl)-3-isobutylpiperazin-2-one (207); (3S,6S)-6-cyclopentyl-4-((5-(4-fluorophenyl)isoxazol-3-yl)methyl)-3-isobutylpiperazin-2-one (208); (3S,6S)-4-((5-(4-chlorophenyl)isoxazol-3-yl)methyl)-6-(cyclopropylmethyl)-3-isobutylpiperazin-2-one (209); (3S,6S)-4-((5-(4-chlorophenyl)isoxazol-3-yl)methyl)-3-isobutyl-6-propylpiperazin-2-one (210); (3S,6S)-4-((5-(4-chlorophenyl)isoxazol-3-yl)methyl)-6-cyclohexyl-3-isobutylpiperazin-2-one (211); (3S,6S)-4-((5-(4-chlorophenyl)isoxazol-3-yl)methyl)-6-cyclopentyl-3-isobutylpiperazin-2-one (212); (3S,6S)-4-((5-(4-fluorophenyl)isoxazol-3-yl)methyl)-3-isobutyl-6-isopropylpiperazin-2-one (213); (3S,6S)-6-((R)-sec-butyl)-4-((5-(4-fluorophenyl)isoxazol-3-yl)methyl)-3-isobutylpiperazin-2-one (214); (3S,6S)-4-((5-(4-fluorophenyl)isoxazol-3-yl)methyl)-3-isobutyl-6-phenylpiperazin-2-one (215); (3S,6S)-6-cyclopropyl-4-((5-(4-fluorophenyl)isoxazol-3-yl)methyl)-3-isobutylpiperazin-2-one (216); (3S,6R)-4-[5-(4-Fluoro-phenyl)-isoxazol-3-ylmethyl]-3-isobutyl-6-methylsulfanylmethyl-piperazin-2-one (217); (3S,6R)-4-((5-(4-chlorophenyl)isoxazol-3-yl)methyl)-3-isobutyl-6-((methylthio)methyl)piperazin-2-one (218); (3S,6S)-4-((5-(4-chlorophenyl)isoxazol-3-yl)methyl)-3-isobutyl-6-phenylpiperazin-2-one (219); (3S,6S)-4-(4-(4-chlorophenyl)thiophene-2-carbonyl)-3,6-diisobutylpiperazin-2-one (272); (3S,6S)-3,6-diisobutyl-4-(5-(4-nitrophenyl)isoxazole-3-carbonyl)piperazin-2-one (273); (3S,6S)-4-(5-(2-chlorophenyl)isoxazole-3-carbonyl)-3,6-diisobutylpiperazin-2-one (274); (3S,6S)-4-(5-(2,4-dichlorophenyl)isoxazole-3-carbonyl)-3,6-diisobutylpiperazin-2-one (275); (3S,6S)-4-(5-(3,4-Dichlorophenyl)isoxazole-3-carbonyl)-3,6-diisobutylpiperazin-2-one (276); (3S,6S)-3,6-diisobutyl-4-(5-(4-(methylthio)phenyl)isoxazole-3-carbonyl)piperazin-2-one (277); (3S,6S)-4-(5-(4-ethylphenyl)isoxazole-3-carbonyl)-3,6-diisobutylpiperazin-2-one (283); (3S,6S)-4-(5-(4-cyclopropylphenyl)isoxazole-3-carbonyl)-3,6-diisobutylpiperazin-2-one (284); (3S,6S)-3,6-diisobutyl-4-(5-phenyl-1,3,4-oxadiazole-2-carbonyl)piperazin-2-one (285); (3S,6S)-4-(5-(3,5-difluorophenyl)isoxazole-3-carbonyl)-3,6-diisobutylpiperazin-2-one (287); (3S,6S)-6-(cyclopropylmethyl)-4-(5-(2,4-difluorophenyl)isoxazole-3-carbonyl)-3-isobutylpiperazin-2-one (288); (3S,6S)-4-(5-(4-fluorophenyl)-1,2,4-oxadiazole-3-carbonyl)-3-isobutyl-6-propylpiperazin-2-one (290); (3S,6S)-6-cyclohexyl-4-(5-(4-fluorophenyl)-1,2,4-oxadiazole-3-carbonyl)-3-isobutylpiperazin-2-one (291); (3S,6S)-4-(5-(4-Fluorophenyl)-1,2,4-oxadiazole-3-carbonyl)-3-isobutyl-6-phenylpiperazin-2-one (292); (3S,6S)-4-(5-(4-Fluorophenyl)-1,2,4-oxadiazole-3-carbonyl)-3,6-diisobutylpiperazin-2-one (293); (3S,6S)-6-Cyclopentyl-4-(5-(4-fluorophenyl)-1,2,4-oxadiazole-3-carbonyl)-3-isobutylpiperazin-2-one (294); (3S,6S)-4-(5-(4-Fluorophenyl)-1,2,4-oxadiazole-3-carbonyl)-3-isobutyl-6-isopropylpiperazin-2-one (295); (3S,6R)-4-(5-(4-Fluorophenyl)-1,2,4-oxadiazole-3-carbonyl)-3-isobutyl-6-((methylthio)methyl)piperazin-2-one (296); (3S,6S)-4-(3-(4-chlorophenyl)isoxazole-5-carbonyl)-3-isobutyl-6-propylpiperazin-2-one (313); (3S,6S)-4-(3-(4-chlorophenyl)isoxazole-5-carbonyl)-6-cyclopentyl-3-isobutyl-piperazin-2-one (314); (3S,6S)-4-(3-(4-chlorophenyl)isoxazole-5-carbonyl)-6-cyclohexyl-3-isobutylpiperazin-2-one (315); (3S,6S)-4-(3-(4-chlorophenyl)isoxazole-5-carbonyl)-3-isobutyl-6-phenylpiperazin-2-one (316); (S)-4-(5-(4-fluorophenyl)isoxazole-3-carbonyl)-3,6-diisobutyl-3,4-dihydropyrazin-2(1H)-one (317); (3S,6S)-4-(5-(4-fluorophenyl)isoxazole-3-carbonyl)-3-isobutyl-1-methyl-6-phenylpiperazin-2-one (321); (3S,6S)-1-ethyl-4-(5-(4-fluorophenyl)isoxazole-3-carbonyl)-3-isobutyl-6-phenylpiperazin-2-one (322); (3S,6S)-4-(5-(4-fluorophenyl)isoxazole-3-carbonyl)-3-isobutyl-6-phenyl-1-propylpiperazin-2-one (323); (3S,6S)-3-(cyclopropylmethyl)-4-(5-(4-fluorophenyl)isoxazole-3-carbonyl)-6-phenylpiperazin-2-one (336); (3S,6S)-3-((R)-sec-butyl)-4-(5-(4-fluorophenyl)isoxazole-3-carbonyl)-6-phenylpiperazin-2-one (339); (3S,6S)-4-(5-(4-fluorophenyl)isoxazole-3-carbonyl)-3-neopentyl-6-phenylpiperazin-2-one (342); (3S,6S)-6-(4-fluorophenyl)-4-(5-(4-fluorophenyl)isoxazole-3-carbonyl)-3-isobutyl-piperazin-2-one (345); (3S,6S)-4-(5-(4-fluorophenyl)isoxazole-3-carbonyl)-3-isobutyl-6-(p-tolyl)piperazin-2-one (348); (3S,6S)-4-(1-(4-chlorophenyl)-1H-1,2,3-triazole-4-carbonyl)-3,6-diisobutylpiperazin-2-one (350); (3S,6S)-4-(1-(4-fluorophenyl)-1H-1,2,3-triazole-4-carbonyl)-3,6-diisobutylpiperazin-2-one (351); (3S,6S)-6-(2-chlorophenyl)-4-(5-(4-fluorophenyl)isoxazole-3-carbonyl)-3-isobutyl-piperazin-2-one (352); (3S,6S)-6-(2-fluorophenyl)-4-(5-(4-fluorophenyl)isoxazole-3-carbonyl)-3-isobutyl-piperazin-2-one (355); (3S,6S)-6-(4-chlorophenyl)-4-(5-(4-fluorophenyl)isoxazole-3-carbonyl)-3-isobutyl-piperazin-2-one(358); (3S,6S)-4-(5-(4-fluorophenyl)isoxazole-3-carbonyl)-3-isobutyl-6-(o-tolyl)piperazin-2-one (361); (3S,6S)-4-(5-(4-fluorophenyl)isoxazole-3-carbonyl)-3-isobutyl-6-(4-(trifluoromethyl)-phenyl)piperazin-2-one (366); (3S,6S)-6-(3-fluorophenyl)-4-(5-(4-fluorophenyl)isoxazole-3-carbonyl)-3-isobutylpiperazin-2-one (367); (3S,6S)-6-(2-fluorophenyl)-4-(5-(4-fluorophenyl)-1,2,4-oxadiazole-3-carbonyl)-3-isobutylpiperazin-2-one (370); (3S,6S)-6-(2-chlorophenyl)-4-(5-(4-fluorophenyl)-1,2,4-oxadiazole-3-carbonyl)-3-isobutylpiperazin-2-one (371); (3S,6S)-6-(2-fluorophenyl)-4-(3-(4-fluorophenyl)isoxazole-5-carbonyl)-3-isobutylpiperazin-2-one (372); (3S,6S)-6-(2-fluorophenyl)-4-((5-(4-fluorophenyl)isoxazol-3-yl)methyl)-3-isobutyl-piperazin-2-one (375); (3S,6S)-4-(4-fluoro-5-(4-fluorophenyl)isoxazole-3-carbonyl)-3-isobutyl-6-phenyl-piperazin-2-one (376); (3S,6S)-4-(4-fluoro-5-(4-fluorophenyl)isoxazole-3-carbonyl)-3,6-diisobutylpiperazin-2-one (379); (3S,6S)-4-(4-fluoro-5-(4-fluorophenyl)isoxazole-3-carbonyl)-6-(2-fluorophenyl)-3-isobutylpiperazin-2-one (380); (3S,6S)-6-cyclopentyl-4-(4-fluoro-5-(4-fluorophenyl)isoxazole-3-carbonyl)-3-isobutylpiperazin-2-one (381); (3S,6S)-6-cyclohexyl-4-(4-fluoro-5-(4-fluorophenyl)isoxazole-3-carbonyl)-3-isobutylpiperazin-2-one (382); (3S,6S)-4-(5-(2,4-difluorophenyl)-4-fluoroisoxazole-3-carbonyl)-3,6-diisobutyl-piperazin-2-one (383); (3S,6S)-4-(5-(2,4-difluorophenyl)-4-fluoroisoxazole-3-carbonyl)-3-isobutyl-6-phenylpiperazin-2-one (385); (3S,6S)-4-(5-(2,4-difluorophenyl)-4-fluoroisoxazole-3-carbonyl)-6-(2-fluorophenyl)-3-isobutylpiperazin-2-one (386); (3S,6S)-4-(5-(2,4-difluorophenyl)-4-fluoroisoxazole-3-carbonyl)-3-isobutyl-6-propylpiperazin-2-one (387); (3S,6S)-6-cyclopentyl-4-(5-(2,4-difluorophenyl)-4-fluoroisoxazole-3-carbonyl)-3-isobutylpiperazin-2-ono (388); (3S,6S)-6-cyclohexyl-4-(5-(2,4-difluorophenyl)-4-fluoroisoxazole-3-carbonyl)-3-isobutylpiperazin-2-one (389); (3S,6S)-4-(5-(3,4-difluorophenyl)-4-fluoroisoxazole-3-carbonyl)-3-isobutyl-6-phenylpiperazin-2-one (390); (3S,6S)-4-(5-(3,4-difluorophenyl)-4-fluoroisoxazole-3-carbonyl)-6-(2-fluorophenyl)-3-isobutylpiperazin-2-one (392); (3S,6S)-4-(5-(3,4-difluorophenyl)-4-fluoroisoxazole-3-carbonyl)-3-isobutyl-6-propylpiperazin-2-one (393); (3S,6S)-4-(5-(3,4-difluorophenyl)-4-fluoroisoxazole-3-carbonyl)-3,6-diisobutyl-piperazin-2-one (394); (3S,6S)-6-cyclopentyl-4-(5-(3,4-difluorophenyl)-4-fluoroisoxazole-3-carbonyl)-3-isobutylpiperazin-2-one (395); (3S,6S)-6-cyclohexyl-4-(5-(3,4-difluorophenyl)-4-fluoroisoxazole-3-carbonyl)-3-isobutylpiperazin-2-one (396); (3S,6S)-4-(5-(4-fluorophenyl)isoxazole-3-carbonyl)-6-phenyl-3-propylpiperazin-2-one (397); (3S,6S)-4-(5-(4-fluorophenyl)isoxazole-3-carbonyl)-3-isopropyl-6-phenylpiperazin-2-one (400); (3S,6S)-4-(5-(4-fluorophenyl)-1,2,4-oxadiazole-3-carbonyl)-3-isopropyl-6-phenyl-piperazin-2-ono (403); (3S,6S)-4-(3-(4-fluorophenyl)-1,2,4-oxadiazole-5-carbonyl)-3-isopropyl-6-phenyl-piperazin-2-one (404); (3S,6S)-4-(5-(4-chlorophenyl)-1,2,4-oxadiazole-3-carbonyl)-3,6-diisobutylpiperazin-2-one (405); (3S,6S)-4-(5-(4-chlorophenyl)-1,2,4-oxadiazole-3-carbonyl)-3-isobutyl-6-phenyl-piperazin-2-one (406); (3S,6S)-3-allyl-4-(5-(4-fluorophenyl)isoxazole-3-carbonyl)-6-phenylpiperazin-2-one (407); (3S,6S)-3-allyl-4-(5-(4-fluorophenyl)isoxazole-3-carbonyl)-6-propylpiperazin-2-one (409); (3S,7S)-4-(5-(4-fluorophenyl)isoxazole-3-carbonyl)-3,7-diisobutyl-1,4-diazepan-2-one (411); (3S)-4-(5-(4-fluorophenyl)isoxazole-3-carbonyl)-3,6-diisobutyl-1,4-diazepan-2-one (413); (3S,7S)-4-(5-(4-fluorophenyl)isoxazole-3-carbonyl)-3-isobutyl-7-phenyl-1,4-diazepan-2-one (415); (3S,6S)-3-isobutyl-6-phenyl-4-(5-(4-fluorophenyl)isoxazole-3-carbonyl)-1,4-diazepan-2-one (420); (3S,6S)-4-(5-(4-fluorophenyl)isoxazole-3-carbonyl)-3-isobutyl-6-((E)-prop-1-en-1-yl)piperazin-2-one (424); (3S,6S)-4-((5-(4-fluorophenyl)isoxazol-3-yl)methyl)-3-isobutyl-6-((E)-prop-1-en-1-yl)piperazin-2-one (425); (3S,6R)-4-(5-(4-fluorophenyl)isoxazole-3-carbonyl)-3-isobutyl-6-(thiophen-2-yl)piperazin-2-one (426); (3S,6S)-4-(5-(4-fluorophenyl)isoxazole-3-carbonyl)-3-isobutyl-6-(thiophen-2-yl)piperazin-2-one (428); (3S,6R)-4-(5-(4-fluorophenyl)-1,2,4-oxadiazole-3-carbonyl)-3-isobutyl-6-(thiophen-2-yl)piperazin-2-one (430); (3S,6R)-4-(3-(4-fluorophenyl)-1,2,4-oxadiazole-5-carbonyl)-3-isobutyl-6-(thiophen-2-yl)piperazin-2-one (431); (3S,6R)-4-(3-(4-fluorophenyl)isoxazole-5-carbonyl)-3-isobutyl-6-(thiophen-2-yl)piperazin-2-one (432); (3S,6R)-4-((5-(4-fluorophenyl)isoxazol-3-yl)methyl)-3-isobutyl-6-(thiophen-2-yl)piperazin-2-one (435); (3S,6S)-4-(5-(4-fluorophenyl)isoxazole-3-carbonyl)-3-isobutyl-6-(thiophen-3-yl)piperazin-2-one (436); (3S,6R)-4-(5-(4-fluorophenyl)-1,2,4-oxadiazole-3-carbonyl)-3-isobutyl-6-(3-methylthiophen-2-yl)piperazin-2-one (441); (3S,6S)-4-((5-(4-fluorophenyl)isoxazol-3-yl)methyl)-3-isobutyl-6-(thiophen-3-yl)piperazin-2-one (442); (3S,6S)-6-cyclobutyl-4-(5-(4-fluorophenyl)isoxazole-3-carbonyl)-3-isobutylpiperazin-2-one (443); (3S,6S)-4-(5-(4-fluorophenyl)-1,2,4-oxadiazole-3-carbonyl)-3-isobutyl-6-(thiophen-3-yl)piperazin-2-one (446); (3S,6S)-4-(3-(4-fluorophenyl)isoxazole-5-carbonyl)-3-isobutyl-6-((E)-prop-1-en-1-yl)piperazin-2-one (447); (3S,6S)-4-(5-(4-fluorophenyl)-1,2,4-oxadiazole-3-carbonyl)-3-isobutyl-6-((E)-prop-1-en-1-yl)piperazin-2-one (448); (3S,6S)-4-(3-(4-fluorophenyl)isoxazole-5-carbonyl)-3-isobutyl-6-(thiophen-3-yl)piperazin-2-one (450); (3S,6R)-4-(5-(4-fluorophenyl)isoxazole-3-carbonyl)-3-isobutyl-6-(5-methylthiophen-2-yl)piperazin-2-one (453); (3S)-4-(5-(4-fluorophenyl)isoxazole-3-carbonyl)-3-isobutyl-6-(tetrahydro-2H-pyran-4-yl)piperazin-2-one (456); (3R,6S)-4-(5-(4-fluorophenyl)isoxazole-3-carbonyl)-3-((methylthio)ethyl)-6-phenylpiperazin-2-one (458); (3S,6S)-3-allyl-4-(5-(4-fluorophenyl)-1,2,4-oxadiazole-3-carbonyl)-6-phenyl-piperazin-2-one (465); (3R,6S)-4-(5-(4-fluorophenyl)-1,2,4-oxadiazole-3-carbonyl)-3-((methylthio)methyl)-6-phenylpiperazin-2-one (466); (3S,6S)-6-cyclobutyl-4-(5-(4-fluorophenyl)-1,2,4-oxadiazole-3-carbonyl)-3-isobutylpiperazin-2-one (467); (3R,6S)-4-(5-(4-fluorophenyl)isoxazole-3-carbonyl)-3-((methylthio)methyl)-6-phenylpiperazin-2-one (468); (3R,6S)-4-(5-(4-fluorophenyl)isoxazole-3-carbonyl)-6-phenyl-3-(thiophen-2-yl)piperazin-2-one (471); (3R,6S)-4-(5-(4-fluorophenyl)-1,2,4-oxadiazole-3-carbonyl)-6-phenyl-3-(thiophen-2-yl)piperazin-2-one (472); (3S,6R)-4-(5-(4-fluorophenyl)-1,2,4-oxadiazole-3-carbonyl)-3-isobutyl-6-(tetrahydro-2H-pyran-4-yl)piperazin-2-one (473); (3S,6S)-4-(5-(4-fluorophenyl)-1,2,4-oxadiazole-3-carbonyl)-3-isobutyl-6-(tetrahydro-2H-pyran-4-yl)piperazin-2-one (474); (3S,6S)-4-(5-(4-fluorophenyl)isoxazole-3-carbonyl)-6-(furan-2-yl)-3-isobutyl-piperazin-2-one (483); (3S,6R)-4-(5-(4-fluorophenyl)isoxazole-3-carbonyl)-6-(furan-2-yl)-3-isobutylpiperazin-2-one (484); (3S,6S)-6-(5-chlorothiophen-2-yl)-4-(5-(4-fluorophenyl)isoxazole-3-carbonyl)-3-isobutylpiperazin-2-one (485); (3S,6R)-6-(5-chlorothiophen-2-yl)-4-(5-(4-fluorophenyl)isoxazole-3-carbonyl)-3-isobutylpiperazin-2-one (488); (3S,6R)-6-(5-chlorothiophen-2-yl)-4-(5-(4-fluorophenyl)isoxazole-3-carbonyl)-3-isobutylpiperazin-2-one (489); (3S,6R)-4-(5-(4-fluorophenyl)isoxazole-3-carbonyl)-3-propyl-6-(thiophen-2-yl)piper-azin-2-one (491); (3S,6R)-4-(5-(4-fluorophenyl)isoxazole-3-carbonyl)-3-propyl-6-(thiophen-2-yl)piperazin-2-one (494); (3S,6S)-6-(2-chlorothiophen-3-yl)-4-(5-(4-fluorophenyl)isoxazole-3-carbonyl)-3-isobutylpiperazin-2-one (496); (3S,6S)-6-(2-chlorothiophen-3-yl)-4-(5-(4-fluorophenyl)-1,2,4-oxadiazole-3-carbonyl)-3-isobutylpiperazin-2-one (499); (2R,5S)-4-(5-(4-fluorophenyl)isoxazole-3-carbonyl)-5-isobutyl-6-oxopiperazine-2-carboxylic acid (501); (3S,6S)-4-(5-(4-fluorophenyl)isoxazole-3-carbonyl)-3-isobutyl-6-vinylpipe-razin-2-one (506); (2R,5S)-4-(5-(4-fluorophenyl)isoxazole-3-carbonyl)-5-isobutyl-N,N-dimethyl-6-oxopiperazine-2-carboxamide (507); (2R,5S)-4-(5-(4-fluorophenyl)isoxazole-3-carbonyl)-5-isobutyl-N-methyl-6-oxopiperazine-2-carboxamide (508); (2R,5S)—N-(2,2-dimethoxyethyl)-4-(5-(4-fluorophenyl)isoxazole-3-carbonyl)-5-isobutyl-6-oxopiperazine-2-carboxamide (509); (3S,6R)-4-(5-(4-fluorophenyl)isoxazole-3-carbonyl)-3-isobutyl-6-(oxazol-2-yl)piperazin-2-one (510); (3S,6R)-4-(5-(4-fluorophenyl)isoxazole-3-carbonyl)-3-isobutyl-6-(oxazol-5-yl)piperazin-2-one (516); (3S,6R)-4-(5-(4-fluorophenyl)-1,2,4-oxadiazole-3-carbonyl)-3-isobutyl-6-(oxazol-5-yl)piperazin-2-one (518); (3S,6R)-4-(3-(4-fluorophenyl)isoxazole-5-carbonyl)-3-isobutyl-6-(oxazol-5-yl)piperazin-2-one (519); (3S,6R)-4-(5-(2,4-difluorophenyl)isoxazole-3-carbonyl)-3-isobutyl-6-(oxazol-5-yl)piperazin-2-one (522); (3S,6R)-4-(5-(4-fluorophenyl)isoxazole-3-carbonyl)-3-isobutyl-6-(1H-pyrrol-2-yl)piperazin-2-one (523); (3S,6S)-4-(5-(4-fluorophenyl)isoxazole-3-carbonyl)-3-isobutyl-6-(1H-pyrrol-2-yl)piperazin-2-one (524); (2R,5S)-1-(5-(4-fluorophenyl)isoxazole-3-carbonyl)-5-isobutyl-2-phenylimidazolidin-4-one (528); (2S,5S)-1-(5-(4-fluorophenyl)isoxazole-3-carbonyl)-5-isobutyl-2-phenylimidazolidin-4-one (529); (3S,6S)-4-(3-(4-fluorophenyl)propioloyl)-3,6-diisobutylpiperazin-2-one (113); 3,6-Diisobutyl-4-(3-phenyl-propynoyl)-piperazin-2-one (114); 3,6-Diisobutyl-4-(3-p-tolyl-propynoyl)-piperazin-2-one (115); 4-[3-(3,4-Difluoro-phenyl)-propynoyl]-3,6-diisobutyl-piperazin-2-one (116); 4-[3-(2,4-Difluoro-phenyl)-propynoyl]-3,6-diisobutyl-piperazin-2-one (117); 3,6-Diisobutyl-4-[3-(4-methoxy-phenyl)-propynoyl]-piperazin-2-one (118); 4-[3-(4-Chloro-2-fluoro-phenyl)-propynoyl]-3,6-diisobutyl-piperazin-2-one (119); 4-[3-(4-Chloro-phenyl)-propynoyl]-3,6-diisobutyl-piperazin-2-one (120); (3S,6S)-3,6-Diisobutyl-4-[3-(3,4,5-trifluoro-phenyl)-propynoyl]-piperazin-2-one (121); (3S,6S)-3,6-Diisobutyl-4-(3-(4-(trifluoromethyl)phenyl)propioloyl)piperazin-2-one (122); (3S,6S)-3,6-diisobutyl-4-(3-(pyridin-3-yl)propioloyl)piperazin-2-one (262); (3S,6S)-3,6-diisobutyl-4-((1R,2R)-2-phenylcyclopropanecarbonyl)piperazin-2-one (70); (3S,6S)-3-isobutyl-4-((1R,2R)-2-phenylcyclopropanecarbonyl)-6-propylpiperazin-2-one (230); (3S,6S)-6-cyclohexyl-3-isobutyl-4-((1R,2R)-2-phenylcyclopropanecarbonyl)-piperazin-2-one (231); (3S,6S)-6-cyclopentyl-3-isobutyl-4-((1R,2R)-2-phenylcyclopropanecarbonyl)-piperazin-2-one (232); (3S,6S)-3-isobutyl-6-isopropyl-4-((1R,2R)-2-phenylcyclopropanecarbonyl)piperazin-2-one (233); (3S,6S)-3-isobutyl-6-phenyl-4-((1R,2R)-2-phenylcyclopropanecarbonyl)piperazin-2-one (234); (3S,6S)-6-cyclopropyl-3-isobutyl-4-((1R,2R)-2-phenylcyclopropanecarbonyl)-piperazin-2-one (235); (3S,6S)-4-[(1R,2R)-2-(4-Fluoro-phenyl)-cyclopropanecarbonyl]-3,6-diisobutyl-piperazin-2-one (236); (3S,6S)-4-[(1R,2R)-2-(4-Chloro-phenyl)-cyclopropanecarbonyl]-3,6-diisobutyl-piperazin-2-one (237); (3S,6S)-4-[(1R,2R)-2-(3,4-Difluoro-phenyl)-cyclopropanecarbonyl]-3,6-diisobutyl-piperazin-2-one (238); (3S,6S)-3,6-Diisobutyl-4-[(1R,2R)-2-(4-methoxy-phenyl)-cyclopropanecarbonyl]-piperazin-2-one (239); (3S,6S)-4-[(1R,2R)-2-(4-Fluoro-phenyl)-cyclopropanecarbonyl]-3-isobutyl-6-propyl-piperazin-2-one (240); (3S,6S)-6-Cyclohexyl-4-[(1R,2R)-2-(4-fluoro-phenyl)-cyclopropanecarbonyl]-3-isobutyl-piperazin-2-one (241); (3S,6S)-6-Cyclopentyl-4-[(1R,2R)-2-(4-fluoro-phenyl)-cyclopropanecarbonyl]-3-isobutyl-piperazin-2-one (242); (3S,6S)-4-[(1R,2R)-2-(4-Chloro-phenyl)-cyclopropanecarbonyl]-6-cyclopropylmethyl-3-isobutyl-piperazin-2-one (243); (3S,6S)-4-[(1R,2R)-2-(4-Chloro-phenyl)-cyclopropanecarbonyl]-3-isobutyl-6-propyl-piperazin-2-one (244); (3S,6S)-4-[(1R,2R)-2-(4-Chloro-phenyl)-cyclopropanecarbonyl]-6-cyclohexyl-3-isobutyl-piperazin-2-one (245); (3S,6S)-4-[(1R,2R)-2-(4-Chloro-phenyl)-cyclopropanecarbonyl]-6-cyclopentyl-3-isobutyl-piperazin-2-one (246); (3S,6S)-4-[(1R,2R)-2-(4-Fluoro-phenyl)-cyclopropanecarbonyl]-3-isobutyl-6-phenyl-piperazin-2-one (247); (3S,6S)-4-[(1R,2R)-2-(4-Chloro-phenyl)-cyclopropanecarbonyl]-3-isobutyl-6-phenyl-piperazin-2-one (248); (3S,6S)-4-[(1R,2R)-2-(4-tert-Butyl-phenyl)-cyclopropanecarbonyl]-3,6-diisobutyl-piperazin-2-one (249); (3S,6S)-4-[(1R,2R)-2-(4-Chloro-2-fluoro-phenyl)-cyclopropanecarbonyl]-3,6-diisobutyl-piperazin-2-one (250); (3S,6S)-4-[(1R,2R)-2-(4-Chloro-2-fluoro-phenyl)-cyclopropanecarbonyl]-6-ethyl-3-isobutyl-piperazin-2-one (251); (3S,6S)-4-[(1R,2R)-2-(4-Chloro-2-fluoro-phenyl)-cyclopropanecarbonyl]-3-isobutyl-6-(1-propyl-butyl)-piperazin-2-one (252); (3S,6S)-4-[(1R,2R)-2-(4-Chloro-2-fluoro-phenyl)-cyclopropanecarbonyl]-6-cyclopentyl-3-isobutyl-piperazin-2-one (253); (3S,6S)-4-[(1R,2R)-2-(4-Chloro-2-fluoro-phenyl)-cyclopropanecarbonyl]-3-isobutyl-6-isopropyl-piperazin-2-one (254); (3S,6S)-4-[(1R,2R)-2-(4-Chloro-2-fluoro-phenyl)-cyclopropanecarbonyl]-3-isobutyl-6-phenyl-piperazin-2-one (255); (3S,6S)-4-[(1R,2R)-2-(2,4-Difluoro-phenyl)-cyclopropanecarbonyl]-3,6-diisobutyl-piperazin-2-one (256); (3S,6S)-4-[(1R,2R)-2-(3,4-Difluoro-phenyl)-cyclopropanecarbonyl]-3-isobutyl-6-propyl-piperazin-2-one (257); (3S,6S)-6-Cyclohexyl-4-[(1R,2R)-2-(3,4-difluoro-phenyl)-cyclopropanecarbonyl]-3-isobutyl-piperazin-2-one (258); (3S,6S)-6-Cyclopentyl-4-[(1R,2R)-2-(3,4-difluoro-phenyl)-cyclopropanecarbonyl]-3-isobutyl-piperazin-2-one (259); (3S,6S)-4-[(1R,2R)-2-(3,4-Difluoro-phenyl)-cyclopropanecarbonyl]-3-isobutyl-6-phenyl-piperazin-2-one (260); (3S,6S)-3-isobutyl-6-neopentyl-4-((1R,2R)-2-phenylcyclopropanecarbonyl)piperazin-2-one (280); (3S,6S)-3,6-dineopentyl-4-((1R,2R)-2-phenylcyclopropanecarbonyl)-piperazin-2-one (281); (3S,6S)-4-[(1R,2R)-2-(4-Fluoro-phenyl)-cyclopropanecarbonyl]-6-cyclopropyl-methyl-3-isobutyl-piperazin-2-one (297); (3S,6S)-4-[(1R,2R)-2-(2,4-fluoro-phenyl)-cyclopropanecarbonyl]-3,6-diisobutyl-piperazin-2-one (298); (3S,6R)-4-((1R,2R)-2-(4-fluorophenyl)cyclopropanecarbonyl)-3-isobutyl-6-((methylthio)methyl)piperazin-2-one (299); (3S,6R)-4-((1R,2R)-2-(4-Chlorophenyl)cyclopropanecarbonyl)-3-isobutyl-6-((methylthio)methyl)piperazin-2-one (300); (3S,6S)-4-((1R,2R)-2-(4-chlorophenyl)cyclopropanecarbonyl)-3-isobutyl-6-phenyl-piperazin-2-one (301); (3S,6S)-4-[(1R,2R)-2-(3,4-Difluoro-phenyl)-cyclopropanecarbonyl]-3-isobutyl-6-isopropyl-piperazin-2-one (309); (3S,6S)-4-((1R,2R)-2-(4-(tert-butyl)phenyl)cyclopropanecarbonyl)-6-cyclopentyl-3-isobutylpiperazin-2-one (310); (3S,6S)-4-((1R,2R)-2-(4-(tert-butyl)phenyl)cyclopropanecarbonyl)-3-isobutyl-6-isopropylpiperazin-2-one (311); (3S,6S)-4-((1R,2R)-2-(4-(tert-butyl)phenyl)cyclopropanecarbonyl)-3-isobutyl-6-phenylpiperazin-2-one (312); (3S,6S)-4-[(1R,2R)-2-(2,4-difluoro-phenyl)-cyclopropanecarbonyl]-6-ethyl-3-isobutyl-piperazin-2-one (324); (3S,6S)-6-cyclopentyl-4-((1R,2R)-2-(2,4-difluorophenyl)cyclopropanecarbonyl)-3-isobutylpiperazin-2-one (325); (3S,6S)-6-cyclohexyl-4-((1R,2R)-2-(2,4-difluorophenyl)cyclopropanecarbonyl)-3-isobutylpiperazin-2-one (326); (3S,6S)-4-[(1R,2R)-2-(2,4-difluoro-phenyl)-cyclopropanecarbonyl]-3-isobutyl-6-phenyl-piperazin-2-one (327); (3S,6S)-4-((1R,2R)-2-(4-(tert-butyl)phenyl)cyclopropanecarbonyl)-6-cyclohexyl-3-isobutylpiperazin-2-one (328); (3S,6S)-4-((1R,2R)-2-(4-bromophenyl)cyclopropanecarbonyl)-3,6-diisobutylpiperazin-2-one (329); (3S,6S)-4-[(1R,2R)-2-(4-Bromo-phenyl)-cyclopropanecarbonyl]-3-isobutyl-6-propyl-piperazin-2-one (331); (3S,6S)-6-cyclopentyl-4-((1R,2R)-2-(4-bromophenyl)cyclopropanecarbonyl)-3-isobutylpiperazin-2-one (332); (3S,6S)-4-[(1R,2R)-2-(4-bromo-phenyl)-cyclopropanecarbonyl]-3-isobutyl-6-phenyl-piperazin-2-one (333); (3S,6S)-4-((1R,2R)-2-(4-(tert-butyl)phenyl)cyclopropanecarbonyl)-3-isobutyl-6-propyl-pylpiperazin-2-one (334); (3S,6S)-6-cyclohexyl-4-((1R,2R)-2-(2,4-difluorophenyl)cyclopropanecarbonyl)-3-isobutylpiperazin-2-one (335); (3S,6S)-3-(cyclopropylmethyl)-6-phenyl-4-((1R,2R)-2-phenylcyclopropanecarbonyl)-piperazin-2-one (338); (3S,6S)-3-((R)-sec-butyl)-6-phenyl-4-((1R,2R)-2-phenylcyclopropanecarbonyl)-piperazin-2-one (341); (3S,6S)-3-neopentyl-6-phenyl-4-((1R,2R)-2-phenylcyclopropanecarbonyl)piperazin-2-one (344); (3S,6S)-6-(4-fluorophenyl)-3-isobutyl-4-((1R,2R)-2-phenylcyclopropanecarbonyl)-piperazin-2-one (347); (3S,6S)-6-(2-chlorophenyl)-3-isobutyl-4-((1R,2R)-2-phenylcyclopropanecarbonyl)-piperazin-2-one (354); (3S,6S)-6-(2-fluorophenyl)-3-isobutyl-4-((1R,2R)-2-phenylcyclopropanecarbonyl)-piperazin-2-one (357); (3S,6S)-6-(4-chlorophenyl)-3-isobutyl-4-((1R,2R)-2-phenylcyclopropanecarbonyl)-piperazin-2-one (360); (3S,6S)-3-isobutyl-4-((1R,2R)-2-phenylcyclopropanecarbonyl)-6-(o-tolyl)piperazin-2-one (363); (3S,6S)-3-isobutyl-4-((1R,2R)-2-phenylcyclopropanecarbonyl)-6-(4-(trifluoro-methyl)phenyl)piperazin-2-one (364); (3S,6S)-6-(3-fluorophenyl)-3-isobutyl-4-((1R,2R)-2-phenylcyclopropanecarbonyl)-piperazin-2-one (369); (3S,6S)-6-(2-fluorophenyl)-4-((1R,2R)-2-(4-fluorophenyl)cyclopropanecarbonyl)-3-isobutylpiperazin-2-one (373); (3S,6S)-6-phenyl-4-((1R,2R)-2-phenylcyclopropanecarbonyl)-3-propylpiperazin-2-one (399); (3S,6S)-3-isopropyl-6-phenyl-4-((1R,2R)-2-phenylcyclopropanecarbonyl)piperazin-2-one (402); (3S,7S)-3-isobutyl-7-phenyl-4-((1R,2R)-2-phenylcyclopropanecarbonyl)-1,4-diazepan-2-one (417); (3S,6S)-3-isobutyl-6-phenyl-4-((1R,2R)-2-phenylcyclopropanecarbonyl)-1,4-diazepan-2-one (418); (3S,6S)-4-[(1S,2S)-2-(2,4-Difluoro-phenyl)-cyclopropanecarbonyl]-3,6-diisobutyl-piperazin-2-one (421); (3S,6S)-3-isobutyl-4-((1R,2R)-2-phenylcyclopropanecarbonyl)-6-((E)-prop-1-en-1-yl)piperazin-2-one (422); (3S,6R)-4-((1R,2R)-2-(4-fluorophenyl)cyclopropanecarbonyl)-3-isobutyl-6-(thiophen-2-yl)piperazin-2-one (433); (3S,6R)-4-((1R,2R)-2-(4-fluorophenyl)cyclopropanecarbonyl)-3-isobutyl-6-(3-methylthiophen-2-yl)piperazin-2-one (438); (3S,6S)-4-((1R,2R)-2-(4-fluorophenyl)cyclopropanecarbonyl)-3-isobutyl-6-((E)-prop-1-en-1-yl)piperazin-2-one (452); (3R,6S)-4-((1R,2R)-2-(4-fluorophenyl)cyclopropanecarbonyl)-3-((methylthio)-methyl)-6-phenylpiperazin-2-one (460); (3S,6S)-3-allyl-4-((1R,2R)-2-(4-fluorophenyl)cyclopropanecarbonyl)-6-phenyl-piperazin-2-one (462); (3S,6S)-6-cyclobutyl-4-((1R,2R)-2-(4-fluorophenyl)cyclopropanecarbonyl)-3-isobutylpiperazin-2-one (464); (3R,6S)-4-((1R,2R)-2-(4-fluorophenyl)cyclopropanecarbonyl)-6-phenyl-3-(thiophen-2-yl)piperazin-2-one (469); (3S,6R)-4-((1R,2R)-2-(4-fluorophenyl)cyclopropanecarbonyl)-3-isobutyl-6-(tetrahy-dro-2H-pyran-4-yl)piperazin-2-one (475); (3S,6S)-4-((1R,2R)-2-(4-fluoro-phenyl)cyclopropanecarbonyl)-3-isobutyl-6-(tetrahydro-2H-pyran-4-yl)piperazin-2-one (476); (3S,6R)-6-(5-chlorothiophen-2-yl)-4-(5-(4-fluorophenyl)isoxazole-3-carbonyl)-3-isobutylpiperazin-2-one (490); (3S,6R)-4-((1R,2R)-2-(4-fluorophenyl)cyclopropanecarbonyl)-3-propyl-6-(thiophen-2-yl)piperazin-2-one (495); (3S,6S)-6-(2-chlorothiophen-3-yl)-4-(1R,2R)-2-(4-fluorophenyl)cyclopropane-carbonyl)-3-isobutylpiperazin-2-one (500); (3S,6R)-3-isobutyl-6-(oxazol-5-yl)-4-((1R,2R)-2-phenylcyclopropane-carbonyl)piperazin-2-one (517); (3S,6R)-4-((1R,2R)-2-(4-fluorophenyl)cyclopropanecarbonyl)-3-isobutyl-6-(oxazol-5-yl)piperazin-2-one (521); (2R,5S)-5-isobutyl-2-phenyl-1-((1S,2S)-2-phenylcyclopropanecarbonyl)imidazolidin-4-one (530); (2R,5S)-5-isobutyl-2-phenyl-1-((1S,2S)-2-phenylcyclopropanecarbonyl)imidazolidin-4-one (531); (2R,5S)-1-((1S,2S)-2-(4-fluorophenyl)cyclopropanecarbonyl)-5-isobutyl-2-phenyl-imidazolidin-4-one (532); (2S,5S)-1-((1S,2S)-2-(4-fluorophenyl)cyclopropane-carbonyl)-5-isobutyl-2-phenyl-imidazolidin-4-one (533); (3S,6S)-3,6-Diisobutyl-4-(4-phenoxybenzoyl)piperazin-2-one (89); (3S,6S)-4-(2-(5-(4-fluorophenyl)isoxazol-3-yl)acetyl)-3,6-diisobutylpiperazin-2-one (278); (3S,6S)-4-(2-(5-(4-fluorophenyl)isoxazol-3-yl)acetyl)-3,6-diisobutylpiperazin-2-one (279); (3S,6S)-4-(Benzofuran-2-carbonyl)-3,6-diisobutyl-piperazin-2-one (76); (3S,6S)-3,6-diisobutyl-4-(7-methoxy-4,5-dihydronaphtho[1,2-b]thiophene-2-carbonyl)piperazin-2-one (286); (3S,6S)-3,6-diisobutyl-4-(7-methoxy-4,5-dihydronaphtho[2,1-d]isoxazole-3-carbonyl)piperazin-2-one (289); (3S,6S)-3,6-Diisobutyl-4-(7-fluoro-4,5-dihydronaphtho[2,1-d]isoxazole-3-carbonyl)piperazin-2-one (302); (3S,6S)-4-(7-fluoro-4,5-dihydronaphtho[2,1-d]isoxazole-3-carbonyl)-3-isobutyl-6-propylpiperazin-2-one (304); (3S,6S)-6-cyclopentyl-4-(7-fluoro-4,5-dihydronaphtho[2,1-d]isoxazole-3-carbonyl)-3-isobutylpiperazin-2-one (305); (3S,6S)-4-(7-fluoro-4,5-dihydronaphtho[2,1-d]isoxazole-3-carbonyl)-3-isobutyl-6-phenylpiperazin-2-one (306); (3S,6S)-3,6-diisobutyl-4-(4H-thieno[3,2-c]chromene-2-carbonyl)piperazin-2-one (307); (3S,6S)-4-(8-fluoro-4H-thieno[3,2-c]chromene-2-carbonyl)-3,6-diisobutylpiperazin-2-one (308); and 2-[(2S,5S)-(2,5-Diisobutyl-3-oxo-piperazin-1-yl)]-N-(2,6-dimethyl-phenyl)-acetamide (88).

12. A composition comprising the compound or its salt thereof according to claim 11 and a pharmaceutically acceptable medium.

13. A composition for treating a hepatitis C virus, which comprises an effective amount of comprising the compound or its salt thereof according to claim 11 and a pharmaceutically acceptable medium.

14. A method of treating a subject infected by a virus, which comprises:
administering to the subject an effective amount of comprising the compound or its salt thereof according to claim 11;
